(19) United States
(12) Patent Application Publication
(10) Pub. No.: US 2025/0098528 A1
(43) Pub. Date: Mar. 20, 2025

(54) ORGANIC ELECTROLUMINESCENT COMPOUND AND ORGANIC ELECTROLUMINESCENT DEVICE

(71) Applicant: DuPont Specialty Materials Korea Ltd., Chungcheongnam-do (KR)

(72) Inventors: Hyun-Woo KANG, Gyeonggi-do (KR); Hong-Se OH, Gyeonggi-do (KR); Soo-Yong LEE, Gyeonggi-do (KR); Hyun KIM, Gyeonggi-do (KR); Young-Rok LIM, Gyeonggi-do (KR); Dong-Hyung LEE, Gyeonggi-do (KR); So-Mi PARK, Gyeonggi-do (KR); Samuel KIM, Gyeonggi-do (KR); Seung-Hoon YOO, Gyeonggi-do (KR); Han-Kyu YOO, Gyeonggi-do (KR); Sang-Ho LEE, Gyeonggi-do (KR); Jin-Man KIM, Gyeonggi-do (KR)

(21) Appl. No.: 18/781,099

(22) Filed: Jul. 23, 2024

(30) Foreign Application Priority Data
Sep. 6, 2023 (KR) 10-2023-0118390
Dec. 7, 2023 (KR) 10-2023-0177058
Jul. 5, 2024 (KR) 10-2024-0088695

(51) Int. Cl. H10K 85/60; C07C 211/61; C07D 307/92; C07D 333/74; H10K 50/15

(52) U.S. Cl. CPC H10K 85/636; C07C 211/61; C07D 307/92; C07D 333/74; H10K 85/631; H10K 50/156; H10K 85/615; H10K 85/622; H10K 85/626; H10K 85/6572; H10K 85/6574; H10K 85/6576

(57) ABSTRACT

The present disclosure relates to an organic electroluminescent compound represented by Formula 1 and an organic electroluminescent device. The organic electroluminescent device according to the present disclosure can exhibit a low driving voltage and a high current efficiency by having different structures of the compounds comprised in the first hole transport layer, the second hole transport layer, and the third hole transport layer and comprising a fluorene-amine, benzofluorene-amine, or naphthofluorene-amine structure in the second hole transport layer.

### TECHNICAL FIELD

The present disclosure relates to an organic electroluminescent compound and an organic electroluminescent device.

### BACKGROUND ART

An electroluminescent device (EL device) is a self-light-emitting display device which has advantages in that it provides a wider viewing angle, a greater contrast ratio, and a faster response time. The first organic EL device was developed by Eastman Kodak in 1987, using small aromatic diamine molecules and aluminum complexes as materials for forming a light-emitting layer (see Appl. Phys. Lett. 51, 913, 1987).

An organic electroluminescent device has a multi-layer structure including a hole injection layer, a hole transport layer, a light-emitting layer, an electron transport layer, and an electron injection layer to increase efficiency and stability. The selection of compounds comprised in the hole transport layer, etc. has been recognized as one of the means to improve the properties of the device, such as hole transport efficiency to the light-emitting layer, luminous efficiency, and lifetime.

In order to improve luminous efficiency, driving voltage, and/or lifespan, various materials or concepts for a hole transport layer of an organic electroluminescent device have been proposed, but they were not proven satisfactory in practical use. Accordingly, there is a continuous demand for the development of an organic electroluminescent device with enhanced performance, such as improved driving voltage, luminous efficiency, power efficiency, and/or lifetime properties, compared to previously disclosed organic electroluminescent devices.

Meanwhile, Japan Patent Application Laid-open No. 2023-021983 and International Publication No. 2015/061198, etc. disclose an amine compound, but do not disclose that the structures of the compounds comprised in the first hole transport layer, the second hole transport layer, and the third hole transport layer of the organic electroluminescent device according to the present disclosure are each different, and the second hole transport layer comprises a compound comprising a fluorene-amine, benzofluorene-amine, or naphthofluorene-amine structure. Therefore, there still is a continuous demand for improving the performance of OLEDs.

### DISCLOSURE OF INVENTION

### Technical Problem

The objective of the present disclosure is firstly, to provide an organic electroluminescent compound which is effective in manufacturing an organic electroluminescent device with improved driving voltage and/or current efficiency, and secondly, to provide an organic electroluminescent device with improved driving voltage, and/or current efficiency.

### Solution to Problem

As a result of intensive studies to solve the technical problems, the present inventors found that the above objective can be achieved by an organic electroluminescent device comprising a first electrode; a second electrode facing the first electrode; a structure in which a hole injection layer, a first hole transport layer, a second hole transport layer, a third transport layer, and a light-emitting layer are sequentially stacked between the first electrode and the second electrode, wherein the structures of the compounds comprised in the first hole transport layer, the second hole transport layer, and the third hole transport layer are each different; wherein the second hole transport layer comprising a compound comprising a fluorene-amine, benzofluorene-amine, or naphthofluorene-amine structure.

In addition, the present inventors found that the above objective can be achieved by an organic electroluminescent compound represented by the following Formula 1.

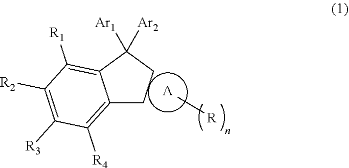

In Formula 1,
ring A represents a substituted or unsubstituted benzene, a substituted or unsubstituted naphthalene, or a substituted or unsubstituted phenanthrene;
Ar₁ and Ar₂ each independently represent a substituted or unsubstituted (C2-C30)alkyl;
R and R₁ to R₄ each independently represent hydrogen, deuterium, halogen, cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, or a substituted or unsubstituted (C1-C30)alkoxy, or may be linked to an adjacent substituent(s) to form a ring(s); with a proviso that at least one of R and R₁ to R₄ is represented by -L₁-NAr₃Ar₄;
L₁ represents a single bond, a substituted or unsubstituted (C6-C30)arylene, or a substituted or unsubstituted (3- to 30-membered)heteroarylene;
Ar₃ and Ar₄ each independently represent a substituted or unsubstituted (C6-C30)aryl, or a substituted or unsubstituted (3- to 30-membered)heteroaryl;
n represents an integer of 1 to 8, if n is plural, each of n may be the same as or different from each other.

In addition, the present inventors found that the above objective can be achieved by an organic electroluminescent compound represented by the following Formula 3.

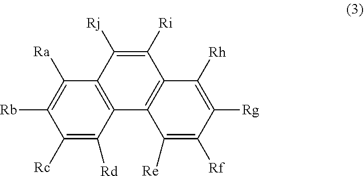

In Formula 3,
at least one of Rₐ to Rⱼ is represented by the following Formula 3-1;

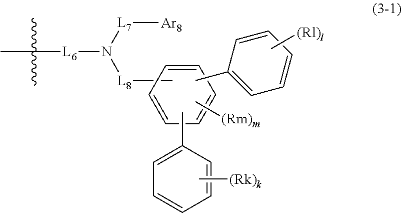

In Formula 3 and Formula 3-1,
Rₐ to Rₘ each independently represent hydrogen, deuterium, halogen, cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl;
[symbol] represents the bond between Formula 3 and Formula 3-1;
L₆ to L₈ each independently represent a single bond, a substituted or unsubstituted (C6-C30)arylene, or a substituted or unsubstituted (3- to 30-membered)heteroarylene;
Ar₈ represents a substituted or unsubstituted (C6-C30)aryl, a substituted or unsubstituted (C3-C30)cycloalkyl, or a substituted or unsubstituted (3- to 30-membered)heteroaryl;
k and l each independently represent an integer of 1 to 5, m represents an integer of 1 to 3;
if k to m represent an integer of 2 or more, each of Rₖ to Rₘ may be the same as or different from each other.

### Advantageous Effects of Invention

An organic electroluminescent device with improved driving voltage and/or current efficiency can be provided by using the organic electroluminescent compound of the present disclosure.

### BRIEF DESCRIPTION OF THE FIGURES

FIG. 1 and FIG. 2 illustrate representative formulas for the organic electroluminescent compound according to the present disclosure.

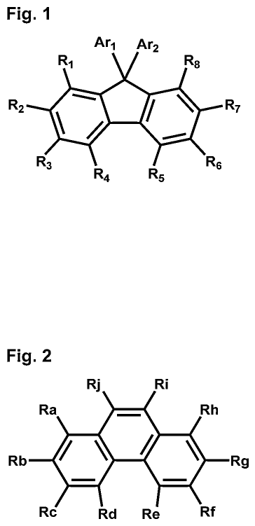

### MODE FOR THE INVENTION

Hereinafter, the present disclosure will be described in detail. However, the following description is intended to explain the present disclosure, and is not meant in any way to restrict the scope of the present disclosure.

The present disclosure relates to an organic electroluminescent device comprising a first electrode; a second electrode facing the first electrode; a structure in which a hole injection layer, a first hole transport layer, a second hole transport layer, a third transport layer, and a light-emitting layer are sequentially stacked between the first electrode and the second electrode, wherein the structures of the compounds comprised in the first hole transport layer, the second hole transport layer, and the third hole transport layer are each different; wherein the second hole transport layer comprises a compound comprising a fluorene-amine, benzofluorene-amine, or naphthofluorene-amine structure.

The term “organic electroluminescent compound” in the present disclosure means a compound that may be used in an organic electroluminescent device, and may be comprised in any layer constituting an organic electroluminescent device, as necessary.

The term “an organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron-blocking material, a light-emitting material (including a host material and a dopant material), an electron buffer material, a hole-blocking material, an electron transport material, an electron injection material, etc. The hole transfer zone material may be at least one selected from the group consisting of a hole transport material, a hole injection material, an electron blocking material, a hole auxiliary material, and a light-emitting auxiliary material.

An organic electroluminescent material of the present disclosure may comprise at least one compound represented by Formula 1 above. The compound of Formula 1 may be comprised in at least one of the layers constituting an organic electroluminescent device, but is not limited thereto, and may be comprised in at least one layer among the layers constituting the hole transfer zone. When the compound of Formula 1 is comprised in a hole transfer layer, a hole auxiliary layer, a electron-blocking layer, a light-emitting layer, or a light-emitting auxiliary layer, it may be comprised as a hole transport material, a hole auxiliary material, an electron-blocking material, a host material, or a light-emitting material.

The term “an electron transport zone” in the present disclosure means an area where electrons move between the light-emitting layer and the cathode. For example, it may comprise at least one of a hole-blocking layer, an electron transport layer, and an electron injection layer, preferably an electron transport layer and an electron injection layer. The hole-blocking layer serves to prevent holes from passing through the light-emitting layer and flowing into the cathode when driving the organic electroluminescent device.

The term “a hole transport zone” in the present disclosure means a region in which holes move between a first electrode and a light-emitting layer, and may comprise, for example, at least one of a hole injection layer, a hole transport layer, a hole auxiliary layer, a light-emitting auxiliary layer, and an electron-blocking layer. The hole injection layer, the hole transport layer, the hole auxiliary layer, the light-emitting auxiliary layer, and the electron-blocking layer may each be a single layer or a multi-layer in which two or more layers, or three or more layers are stacked. According to one embodiment of the present disclosure, the hole transport zone may comprise a first hole transport layer and a second hole transport layer, and may further comprise a third hole transport layer. The second hole transport layer and the third hole transport layer may be at least one of a plurality of hole transport layers, and may comprise at least one of a hole auxiliary layer, a light-emitting auxiliary layer, and an electron-blocking layer. In addition, according to another embodiment of the present disclosure, the hole transport zone may comprise a first hole transport layer and a second hole transport layer, wherein the first hole transport layer may be placed between a first electrode and a light-emitting layer, the second hole transport layer may be placed between the first hole transport layer and the light-emitting layer, and the second hole transport layer may be a layer serving as a hole transport layer, a light-emitting auxiliary layer, a hole auxiliary layer, and/or an electron-blocking layer. According to another embodiment of the present disclosure, the hole transport zone may comprise a first hole transport layer, a second hole transport layer, and a third hole transport layer, wherein the first hole transport layer may be placed between a first electrode and a light-emitting layer, the second hole transport layer may be placed between the first hole transport layer and the light-emitting layer, the third hole transport layer may be placed between the second hole transport layer and the light-emitting layer, and the third hole transport layer may be a layer serving as a hole transport layer, a light-emitting auxiliary layer, a hole auxiliary layer, and/or an electron-blocking layer.

Herein, the term “(C1-C30)alkyl” is meant to refer to a linear or branched alkyl having 1 to 30 carbon atoms constituting a chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc. The term “(C2-C30)alkenyl” in the present disclosure is meant to refer to a linear or branched alkenyl having 2 to 30 carbon atoms constituting the chain, in which the number of carbon atoms is preferably 2 to 20, and more preferably 2 to 10. The above alkenyl may include vinyl, 1-propenyl, 2-propenyl, 1-butenyl, 2-butenyl, 3-butenyl, 2-methylbut-2-enyl, etc. The term “(C2-C30)alkynyl” in the present disclosure is meant to refer to a linear or branched alkynyl having 2 to 30 carbon atoms constituting the chain, in which the number of carbon atoms is preferably 2 to 20, and more preferably 2 to 10. The above alkynyl may include ethynyl, 1-propynyl, 2-propynyl, 1-butynyl, 2-butynyl, 3-butynyl, 1-methylpent-2-ynyl, etc. The term “(C3-C30)cycloalkyl” is meant to refer to a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, etc. The term “(3- to 7-membered)heterocycloalkyl” in the present disclosure is meant to refer to a cycloalkyl having 3 to 7, preferably 5 to 7 ring backbone atoms, and including at least one heteroatom selected from the group consisting of B, N, O, S, Si, and P, preferably at least one heteroatom selected from the group consisting of O, S, and N. The above heterocycloalkyl may include tetrahydrofuran, pyrrolidine, thiolane, tetrahydropyran, etc. The term “(C6-C30)aryl(ene)” and “(C6-C30)arene” in the present disclosure is meant to refer to a monocyclic or fused ring radical derived from an aromatic hydrocarbon having 6 to 30 ring backbone carbon atoms, and may be partially saturated. The number of the ring backbone carbon atoms is preferably 6 to 25, more preferably 6 to 18. The above aryl(ene) may comprise a spiro structure. The above aryl may include phenyl, biphenyl, terphenyl, naphthyl, binaphthyl, phenylnaphthyl, naphthylphenyl, phenylterphenyl, fluorenyl, phenylfluorenyl, diphenylfluorenyl, dimethylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, etc. Specifically, the aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenylyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The term “(3- to 30-membered)heteroaryl(ene)” and “(3- to 30-membered)heteroarene” in the present disclosure is meant to refer to an aryl(ene) having 3 to 30 ring backbone atoms and including at least one heteroatom(s) selected from the group consisting of B, N, O, S, Si, P, Se, Te, and Ge. The number of heteroatoms is preferably 1 to 4. The above heteroaryl(ene) may be a monocyclic ring or a fused ring condensed with at least one benzene ring; may be partially saturated. In addition, the above heteroaryl(ene) may be one formed by linking at least one heteroaryl or aryl group to a heteroaryl(ene) group via a single bond(s), and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, benzophenanthrofuranyl, dibenzothiophenyl, benzimidazolyl, benzothiazolyl, benzoisothiazolyl, benzophenanthrothiophenyl, benzoisoxazolyl, benzoxazolyl, phenanthrooxazolyl, phenanthrothiazolyl, isoindolyl, indolyl, benzoindolyl, indazolyl, benzothiadiazolyl, quinolyl, isoquinolyl, cinnolinyl, quinazolinyl, benzoquinazolinyl, quinoxalinyl, benzoquinoxalinyl, naphthyridinyl, carbazolyl, benzocarbazolyl, dibenzocarbazolyl, phenoxazinyl, phenothiazinyl, phenanthridinyl, benzodioxolyl, dihydroacridinyl, etc. More specifically, the heteroaryl may include 1-pyrrolyl, 2-pyrrolyl, 3-pyrrolyl, pyrazinyl, 2-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 3-pyridyl, 4-pyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl) pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Additionally, “heteroaryl(ene)” can be classified into a heteroaryl(ene) with electronic properties and a heteroaryl(ene) with hole properties. A heteroaryl(ene) with electronic properties is a substituent that is relatively rich in electrons in the parent nucleus, for example, a substituted or unsubstituted pyridinyl, a substituted or unsubstituted pyrimidinyl, a substituted or unsubstituted triazinyl, a substituted or unsubstituted quinazolinyl, a substituted or unsubstituted quinoxalinyl, a substituted or unsubstituted quinolyl, etc. A heteroaryl(ene) with hole properties is a substituent that is relatively electron-deficient in the parent nucleus, for example, a substituted or unsubstituted carbazolyl, a substituted or unsubstituted dibenzofuranyl, a substituted or unsubstituted dibenzothiophenyl, etc. In the present disclosure, the term “halogen” includes F, Cl, Br, and I.

In addition, “ortho-” (“o-”), “meta-” (“m-”), and “para” (“p-”) are prefixes which each represent the relative positions of substituents. The prefix “ortho-” indicates that two substituents are adjacent to each other, and for example, when two substituents in a benzene derivative occupy positions 1 and 2, this is called an “ortho-” configuration. The prefix “meta-” indicates that two substituents are at positions 1 and 3, and for example, when two substituents in a benzene derivative occupy positions 1 and 3, this is called a “meta-” configuration. The prefix “para-” indicates that two substituents are at positions 1 and 4, and for example, when two substituents in a benzene derivative occupy positions 1 and 4, this is called a “para-” configuration.

Herein, “substituted” in the expression “substituted or unsubstituted” means that a hydrogen atom in a certain functional group is replaced with another atom or another functional group, i.e., a substituent. Unless otherwise specified, the substituent may replace hydrogen at a position where the substituent can be substituted without limitation, and when two or more hydrogen atoms in a certain functional group are each replaced with a substituent, each substituent may be the same as or different from each other. The maximum number of substituents that can be substituted for a certain functional group may be the total number of valences that can be substituted for each atom forming the functional group. Herein, the substituted alkyl, the substituted alkenyl, the substituted aryl(ene), the substituted heteroaryl(ene), the substituted cycloalkyl, and the substituted alkoxy each independently are at least one selected from the group consisting of deuterium; a halogen; a cyano; a carboxyl; a nitro; a hydroxyl; a phosphinoxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (C6-C30)aryl unsubstituted or substituted with at least one of a (C1-C30)alkyl(s) and a di(C6-C30)arylamino(s); a (3- to 30-membered)heteroaryl unsubstituted or substituted with at least one of a (C6-C30)aryl(s); a tri(C1-C30)alkylsilyl(s); a tri(C6-C30)arylsilyl(s); a di(C1-C30)alkyl(C6-C30)arylsilyl(s); a (C1-C30)alkyldi(C6-C30)arylsilyl(s); a fused ring group of a (C3-C30) aliphatic ring(s) and a (C6-C30) aromatic ring(s); an amino; a mono- or di(C1-C30)alkylamino; a mono- or di(C2-C30)alkenylamino; a mono- or di(C6-C30)arylamino unsubstituted or substituted with a (C1-C30)alkyl; a mono- or di(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. According to one embodiment of the present disclosure, the substituent(s) each independently may be deuterium; a (C1-C30)alkyl; a (C3-C30)cycloalkyl; a (C3-C30)cycloalkenyl; a (3- to 7-membered)heterocycloalkyl; a (C6-C30)aryl unsubstituted or substituted with at least one of a (C1-C30)alkyl(s) and a di(C6-C30)arylamino(s); a (3- to 30-membered)heteroaryl unsubstituted or substituted with at least one of a (C6-C30)aryl(s); a fused ring group of a (C3-C30) aliphatic ring(s) and a (C6-C30) aromatic ring(s); an amino; a mono- or di(C1-C30)alkylamino; a mono- or di(C2-C30)alkenylamino; a mono- or di(C6-C30)arylamino unsubstituted or substituted with a (C1-C30)alkyl; a mono- or di(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C6-C30)aryl(C1-C30)alkyl; and a (C1-C30)alkyl(C6-C30)aryl. According to another embodiment of the present disclosure, the substituent(s) each independently may be deuterium; a (C1-C20)alkyl; a (C3-C20)cycloalkyl; a (3- to 7-membered)heterocycloalkyl; a (C6-C20)aryl unsubstituted or substituted with at least one of a (C1-C30)alkyl(s) and a di(C6-C30)arylamino(s); a (3- to 20-membered)heteroaryl unsubstituted or substituted with at least one of a (C6-C30)aryl(s); a fused ring group of a (C3-C30) aliphatic ring(s) and a (C6-C30) aromatic ring(s); a mono- or di(C6-C20)arylamino unsubstituted or substituted with a (C1-C30)alkyl; a mono- or di(3- to 20-membered)heteroarylamino; a (C1-C20)alkyl(C6-C20)arylamino; a (C1-C20)alkyl(3- to 20-membered)heteroarylamino; a (C2-C20)alkenyl(C6-C20)arylamino; a (C2-C20)alkenyl(3- to 20-membered)heteroarylamino; a (C6-C20)aryl(3- to 20-membered)heteroarylamino; a (C6-C30)aryl(C1-C30)alkyl; and a (C1-C30)alkyl(C6-C30)aryl. For example, the substituent(s) each independently may be at least one selected from the group consisting of deuterium, a methyl, a naphthyl, a phenanthrenyl, a dibenzofuranyl, a phenyl unsubstituted or substituted with deuterium, a phenanthrylene, a dimethylfluorenyl, a benzonaphthofuranyl, a benzonaphthothiophenyl, a phenyldimethylfluorenylamino, and an adamantyl.

In the present disclosure, if a substituent is not indicated in the chemical formula or compound structure, it may mean that all possible positions for the substituent are hydrogen or deuterium. That is, in the case of deuterium, it is an isotope of hydrogen, and some hydrogen atoms may be the isotope deuterium, and in this case, the content of deuterium may be 0% to 100%. In the present disclosure, in cases where a substituent is not indicated in the chemical formula or compound structure, if the substituent is not explicitly excluded, such as 0% deuterium, 100% hydrogen, and all substituents are hydrogen, hydrogen and deuterium may be used intermixed in a compound. The deuterium is one of the isotopes of hydrogen and an element with a deuteron consisting of one proton and one neutron as its nucleus. It can be represented as hydrogen-2, whose element symbol can also be written as D or 2H. The isotopes are atoms with the same atomic number (Z) but different mass numbers (A), and can also be interpreted as elements with the same number of protons but different numbers of neutrons.

In the present disclosure, “a combination thereof” refers to a combination of one or more elements from the corresponding list to form a known or chemically stable arrangement that can be envisioned by a person skilled in the art from the corresponding list. For example, alkyl and deuterium can be combined to form a partially or fully deuterated alkyl group; halogen and alkyl can be combined to form a halogenated alkyl substituent; and halogen, alkyl, and aryl can be combined to form a halogenated arylalkyl. For example, a preferred combination of substituents includes up to 50 atoms that are not hydrogen or deuterium, or up to 40 atoms that are not hydrogen or deuterium, or up to 30 atoms that are not hydrogen or deuterium, or in many cases, a preferred combination of substituents may comprise up to 20 atoms that are not hydrogen or deuterium.

Hereinafter, an organic electroluminescent device will be described in more detail.

According to one embodiment of the present disclosure, an organic electroluminescent device is provided which comprises a first electrode; a second electrode facing the first electrode; a structure in which a hole injection layer, a first hole transport layer, a second hole transport layer, a third transport layer, and a light-emitting layer are sequentially stacked between the first electrode and the second electrode. Specifically, the organic electroluminescent device according to one embodiment of the present disclosure, the structures of the compounds comprised in the first hole transport layer, the second hole transport layer, and the third hole transport layer are each different; and the second hole transport layer comprises a compound comprising a fluorene-amine, benzofluorene-amine, or naphthofluorene-amine structure.

The compound comprising the fluorene-amine, benzofluorene-amine, or naphthofluorene-amine structure may be represented by the following Formula 1.

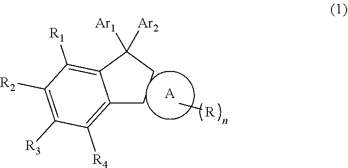

In Formula 1,
ring A represents a substituted or unsubstituted benzene, a substituted or unsubstituted naphthalene, or a substituted or unsubstituted phenanthrene;
Ar₁ and Ar₂ each independently represent a substituted or unsubstituted (C1-C30)alkyl, a substituted or unsubstituted (C6-C30)aryl, or may be linked to each other to form a ring(s);
R and R₁ to R₄ each independently represent hydrogen, deuterium, halogen, cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, or a substituted or unsubstituted (C1-C30)alkoxy, or may be linked to an adjacent substituent(s) to form a ring(s); with a proviso that at least one of R and R₁ to R₄ is represented by -L₁-NAr₃Ar₄;
L₁ represents a single bond, a substituted or unsubstituted (C6-C30)arylene, or a substituted or unsubstituted (3- to 30-membered)heteroarylene;
Ar₃ and Ar₄ each independently represent a substituted or unsubstituted (C6-C30)aryl, or a substituted or unsubstituted (3- to 30-membered)heteroaryl;
n represents an integer of 1 to 8, if n is plural, each of n may be the same as or different from each other.

According to one embodiment of the present disclosure, the compound represented by the Formula 1 may be represented by any one of the following Formulas 1-1 to 1-11.

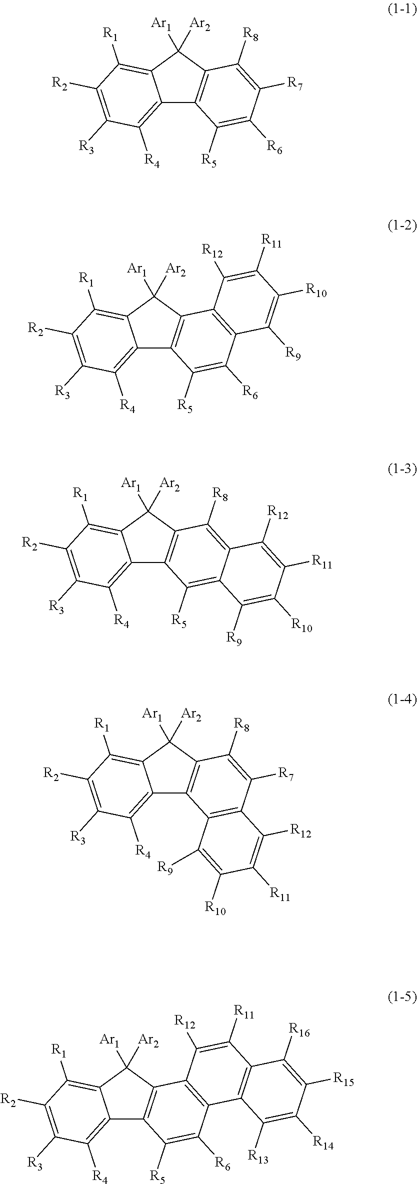

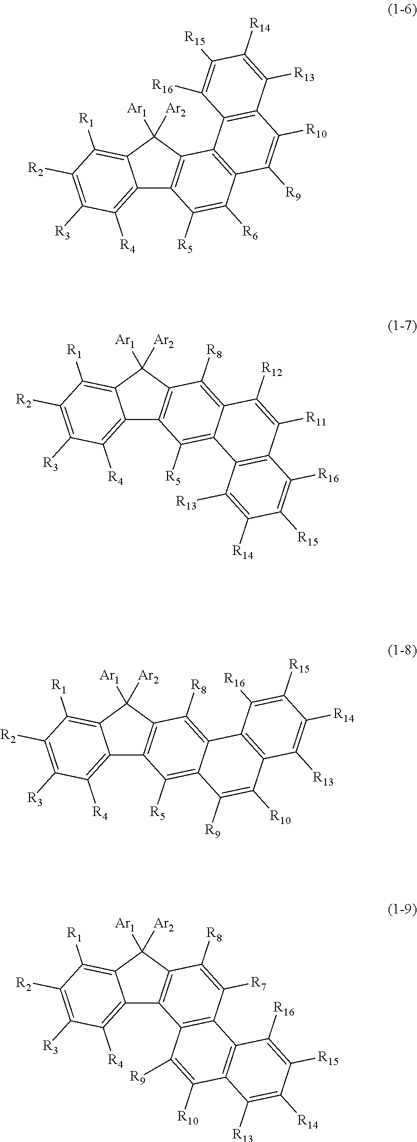

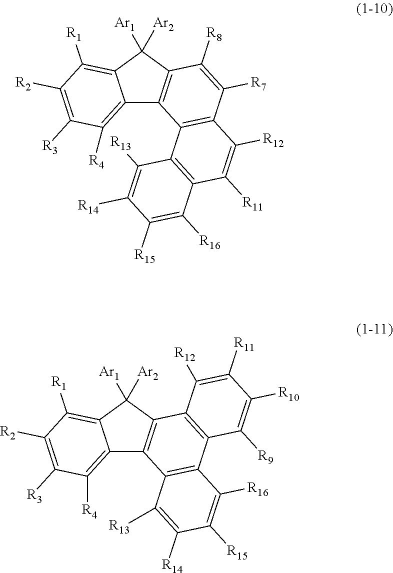

In Formulas 1-1 to 1-11,

Ar₁ and Ar₂ each independently represent a substituted or unsubstituted (C1-C30)alkyl, a substituted or unsubstituted (C6-C30)aryl, or may be linked to each other to form a fused ring(s). According to one embodiment of the present disclosure, Ar₁ and Ar₂ each independently may represent a substituted or unsubstituted (C1-C20)alkyl, a substituted or unsubstituted (C6-C20)aryl, or may be linked to each other to form a fused ring(s). According to another embodiment of the present disclosure, Ar₁ and Ar₂ each independently may represent a substituted or unsubstituted (C1-C10)alkyl, a substituted or unsubstituted (C6-C10)aryl, or may be linked to each other to form a fused ring(s). For example, Ar₁ and Ar₂ each independently may represent a methyl, an ethyl, a propyl, a butyl, a pentyl, a hexyl, a heptyl, an octyl, a phenyl, or may be linked to each other to form a (C13)aryl, and these may be substituted with at least one deuterium.

R₁ to R₁₆ each independently represents hydrogen, deuterium, a halogen, a cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, or a substituted or unsubstituted (C1-C30)alkoxy, or may be linked to an adjacent substituent(s) to form a ring(s), with a proviso that at least one of R₁ to R₁₆ is represented by -L₁-NAr₃Ar₄. According to one embodiment of the present disclosure, R₁ to R₁₆ each independently may represent hydrogen, deuterium, a substituted or unsubstituted (C1-C20)alkyl, a substituted or unsubstituted (C6-C20)aryl, or may be linked to an adjacent substituent(s) to form a ring(s), with a proviso that at least one of R₁ to R₁₆ is represented by -L₁-NAr₃Ar₄. For example, R₁ to R₁₆ each independently may represent hydrogen, deuterium, a phenyl substituted or unsubstituted with deuterium, or -L₁-NAr₃Ar₄.

L₁ represents a single bond, a substituted or unsubstituted (C6-C30)arylene, or a substituted or unsubstituted (3- to 30-membered)heteroarylene. According to one embodiment of the present disclosure, L₁ may represent a single bond, a substituted or unsubstituted (C6-C25)arylene, or a substituted or unsubstituted (3- to 25-membered)heteroarylene. For example, L₁ may represent a single bond, a phenylene, or a phenanthrenylene, and these may be substituted with at least one deuterium.

Ar₃ and Ar₄ each independently represent a substituted or unsubstituted (C6-C30)aryl, or a substituted or unsubstituted (3- to 30-membered)heteroaryl. According to one embodiment of the present disclosure, Ar₃ and Ar₄ each independently represent a substituted or unsubstituted (C6-C30)aryl, or a substituted or unsubstituted (3- to 25-membered)heteroaryl. For example, Ar₃ and Ar₄ each independently may represent a benzonaphthofuranyl; a benzonaphthothiophenyl; a biphenyl substituted or unsubstituted with a methyl; a dibenzofuranyl; a dibenzothiophenyl; a dimethylfluorenyl substituted or unsubstituted with a phenyl; a diethylfluorenyl; a diphenylfluorenyl; a dipropylfluorenyl; a dibutylfluorenyl; a dipentylfluorenyl; a dihexylfluorenyl; a diheptylfluorenyl; a dioctylfluorenyl; a dimethylbenzofluorenyl substituted or unsubstituted with a phenyl; a diethylbenzofluorenyl; a diphenylbenzofluorenyl substituted or unsubstituted with a phenyl; a phenanthrenyl substituted or unsubstituted with a naphthyl, a phenyl substituted or unsubstituted with deuterium, a dibenzofuranyl, or a phenyldimethylfluorenylamino; a phenyl substituted or unsubstituted with a naphthyl, a benzonaphthofuranyl, a benzonaphthothiophenyl, or a methyl; a naphthyl; a quaterphenyl; an o-terphenyl; a m-terphenyl; a p-terphenyl; or a carbazolyl substituted or unsubstituted with a phenyl substituted or unsubstituted with a methyl or adamantyl, a naphthyl substituted or unsubstituted with a methyl, or biphenyl; and these may be substituted with at least one deuterium.

The compound represented by Formula 1 may be at least one selected from the following compounds, but is not limited thereto.

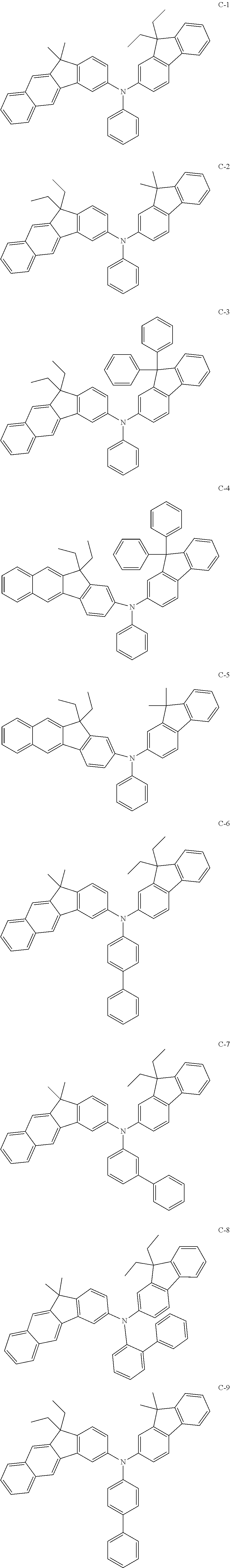

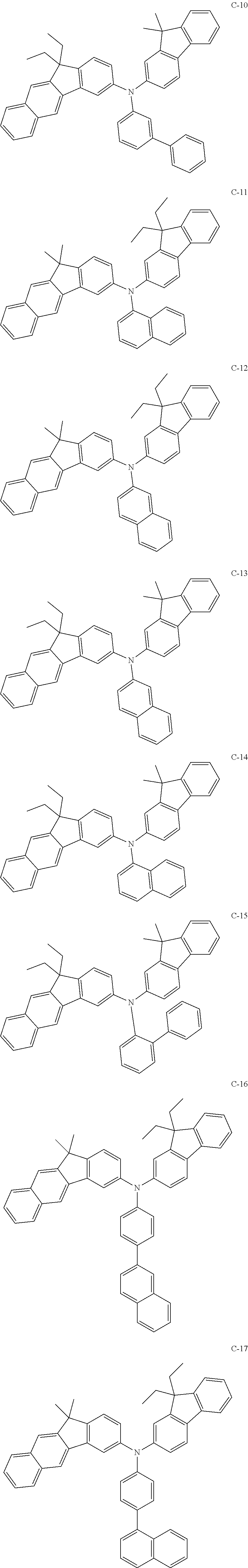

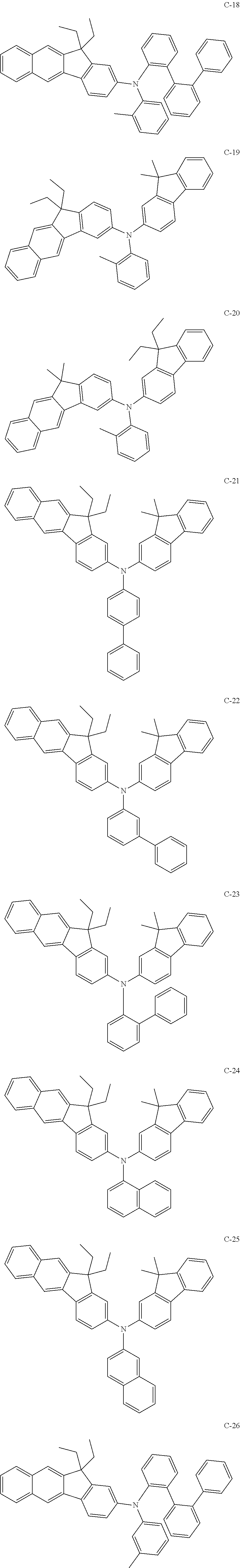

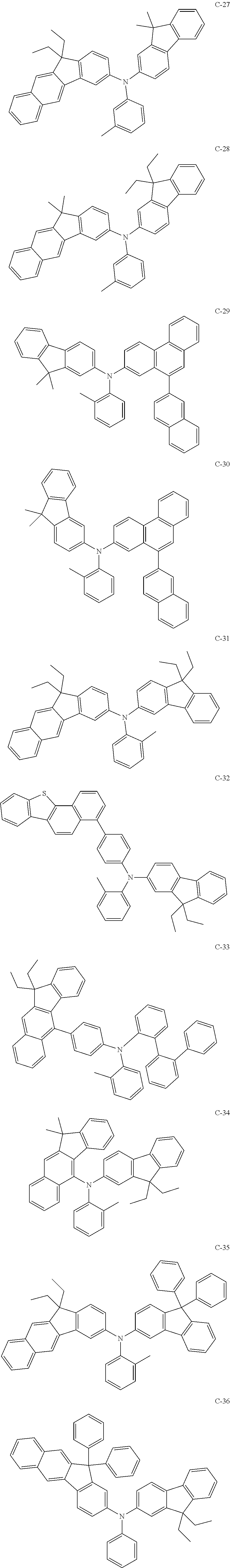

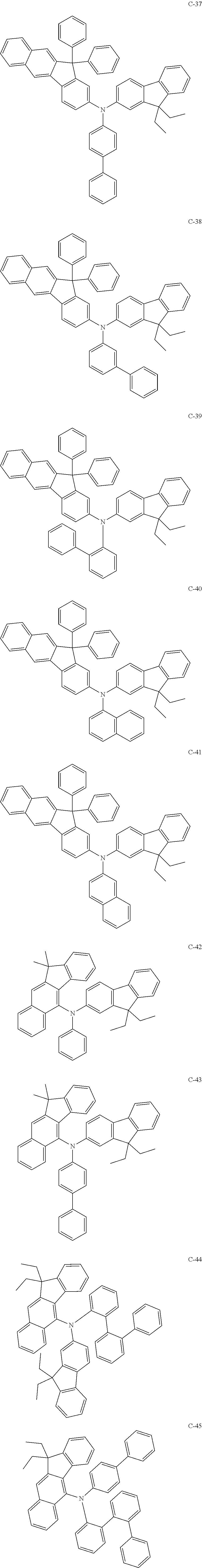

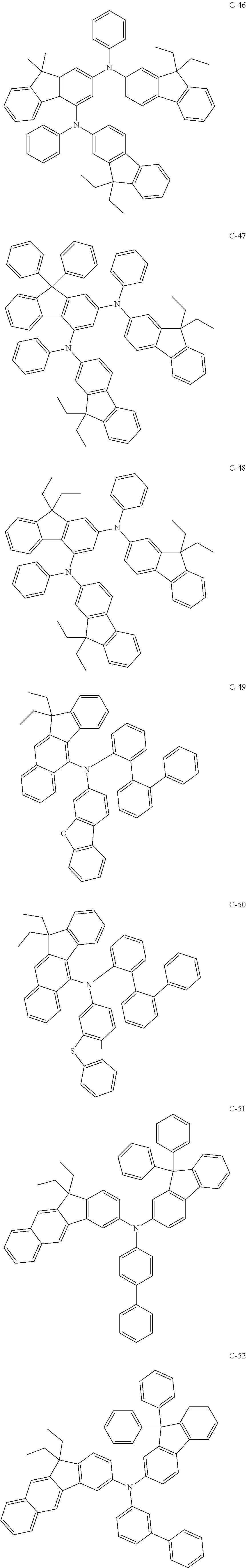

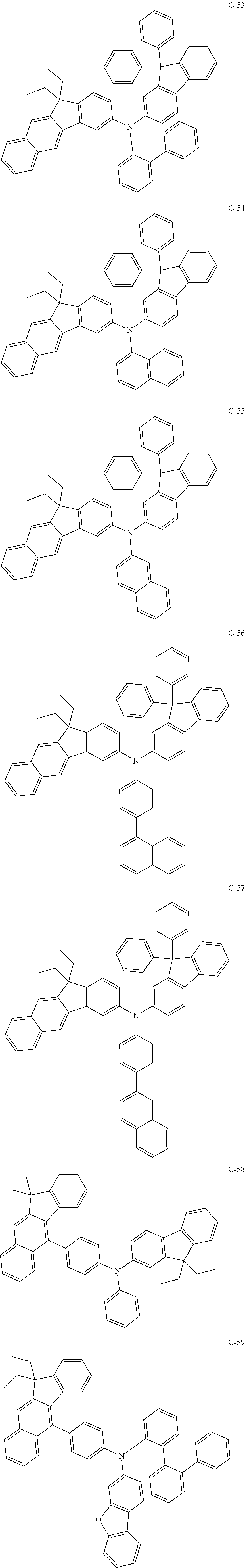

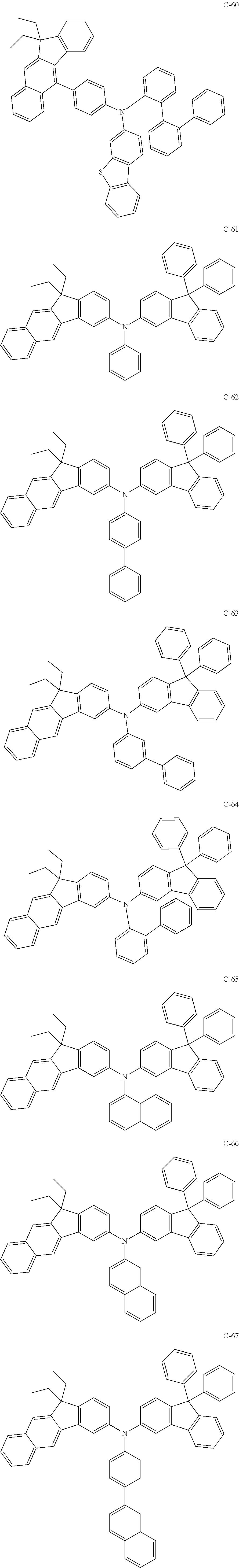

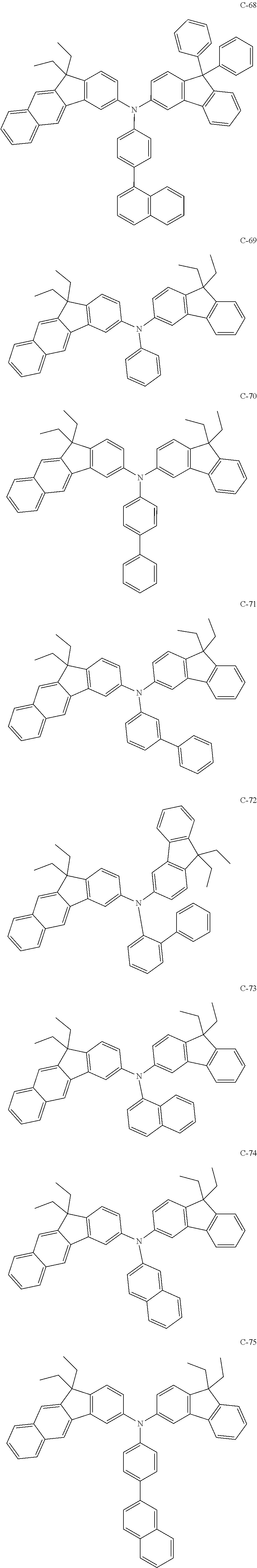

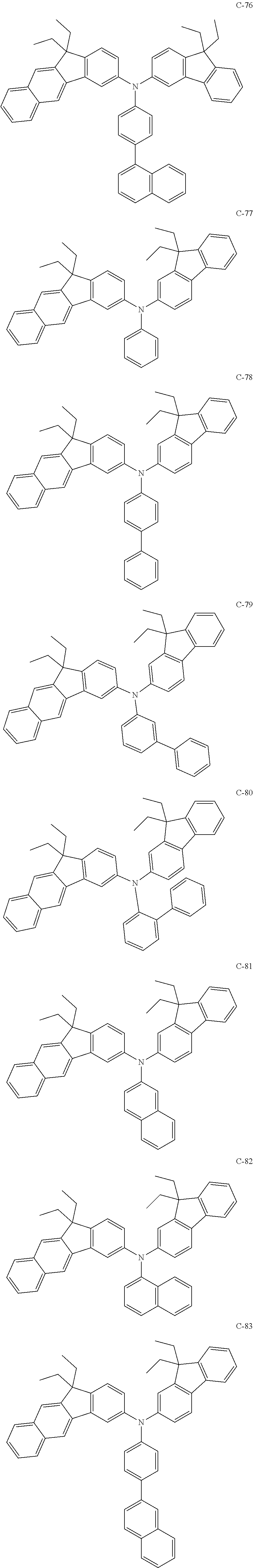

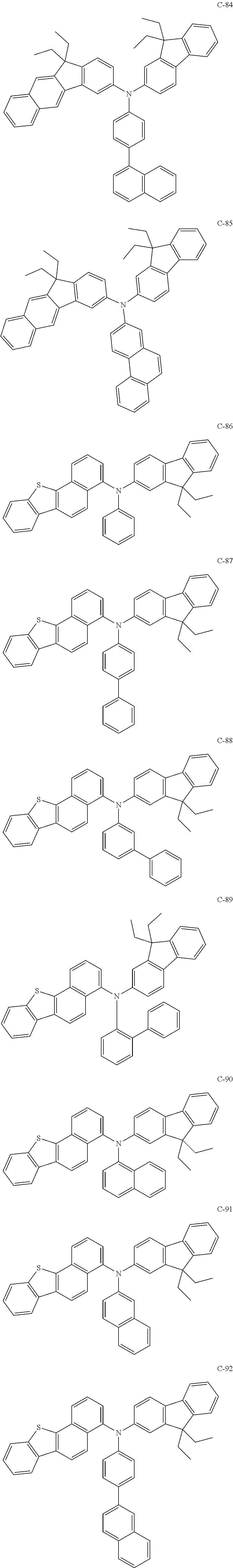

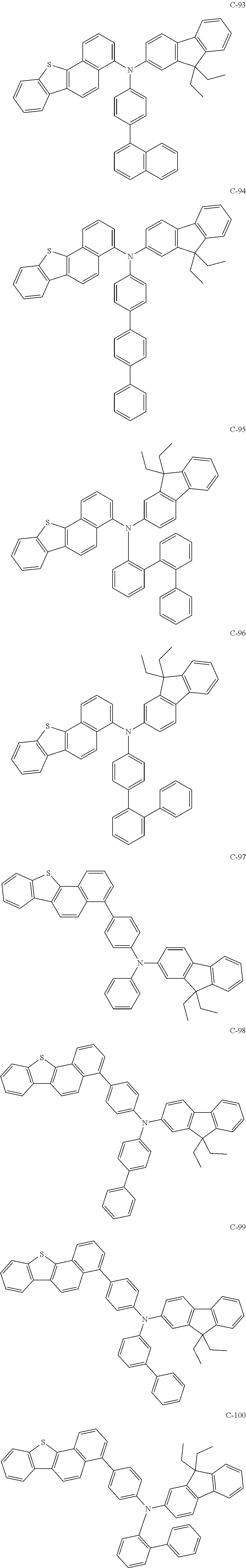

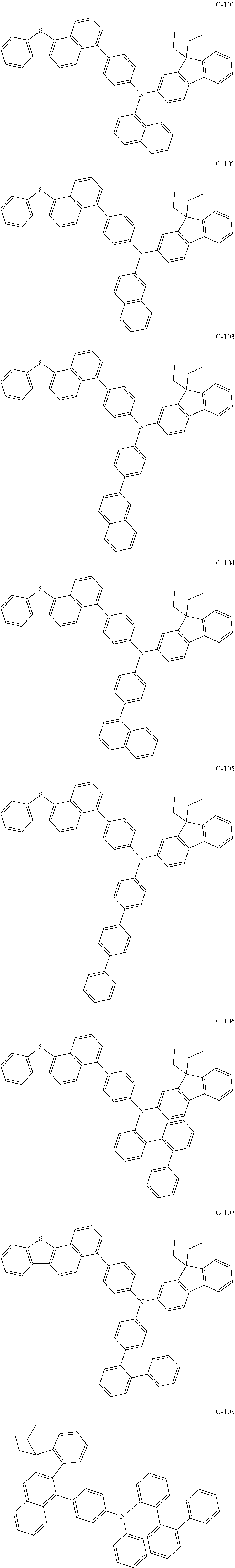

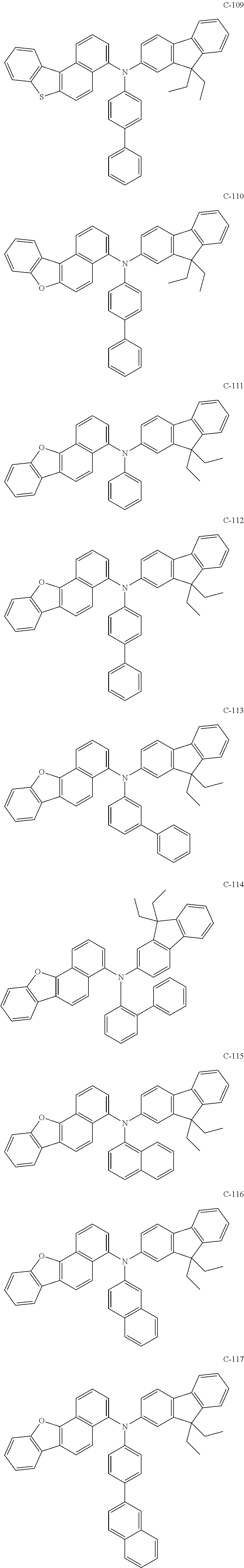

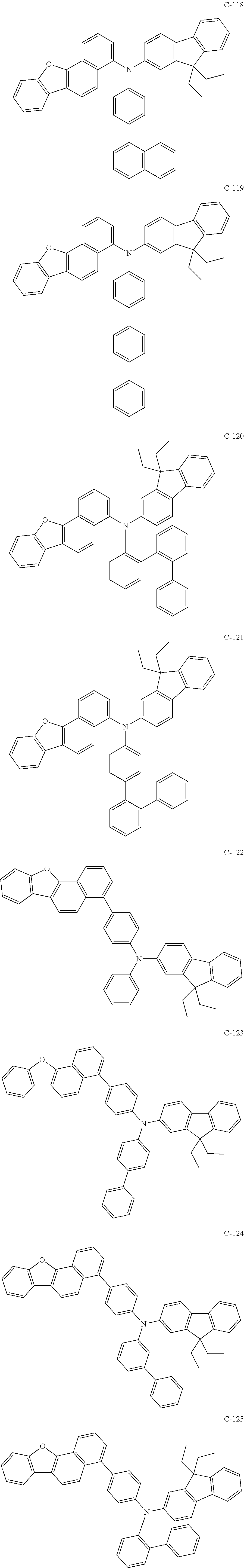

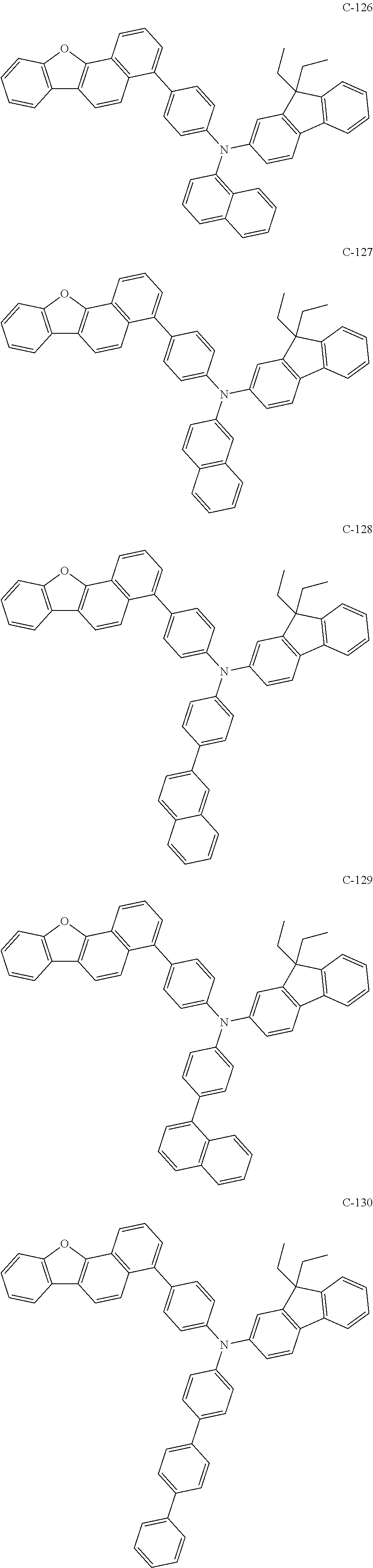 [SMILES: CCC1(CC)c2ccccc2-c2ccc(N(c3ccc(-c4cccc5c4ccc4c6ccccc6oc54)cc3)c3cccc4ccccc34)cc21 ; CCC1(CC)c2ccccc2-c2ccc(N(c3ccc(-c4cccc5c4ccc4c6ccccc6oc54)cc3)c3ccc4ccccc4c3)cc21 ; CCC1(CC)c2ccccc2-c2ccc(N(c3ccc(-c4ccc5ccccc5c4)cc3)c3ccc(-c4cccc5c4ccc4c6ccccc6oc54)cc3)cc21 ; CCC1(CC)c2ccccc2-c2ccc(N(c3ccc(-c4cccc5ccccc45)cc3)c3ccc(-c4cccc5c4ccc4c6ccccc6oc54)cc3)cc21 ; CCC1(CC)c2ccccc2-c2ccc(N(c3ccc(-c4ccc(-c5ccccc5)cc4)cc3)c3ccc(-c4cccc5c4ccc4c6ccccc6oc54)cc3)cc21]

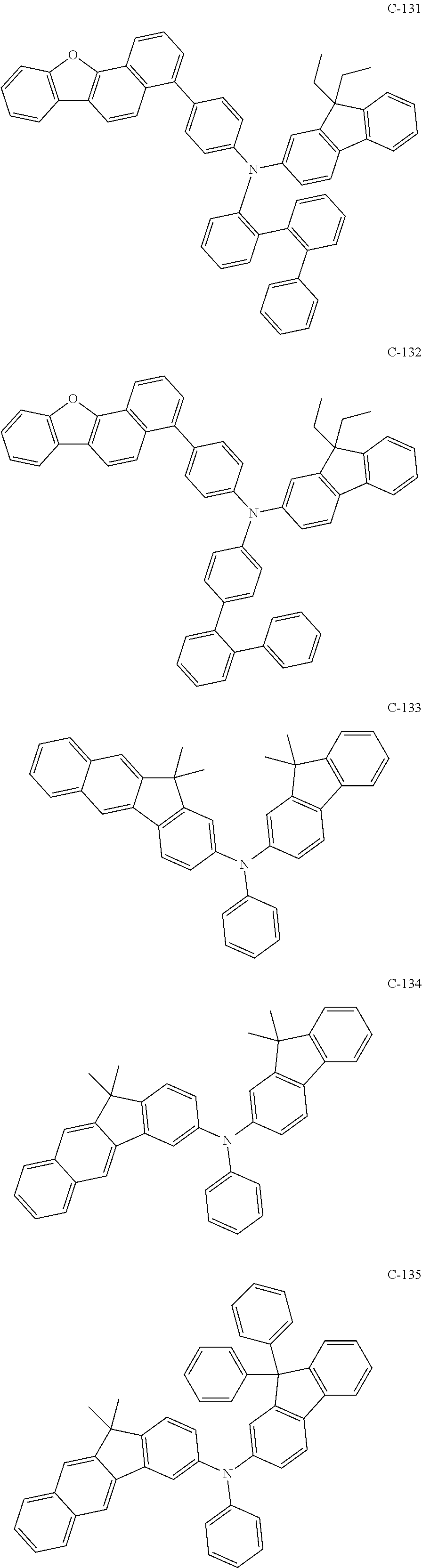 [SMILES: CCC1(CC)c2ccccc2-c2ccc(N(c3ccc(-c4cccc5c4ccc4c6ccccc6oc54)cc3)c3ccccc3-c3ccccc3-c3ccccc3)cc21 ; CCC1(CC)c2ccccc2-c2ccc(N(c3ccc(-c4ccccc4-c4ccccc4)cc3)c3ccc(-c4cccc5c4ccc4c6ccccc6oc54)cc3)cc21 ; CC1(C)c2ccccc2-c2ccc(N(c3ccccc3)c3ccc4c(c3)C(C)(C)c3cc5ccccc5cc3-4)cc21 ; CC1(C)c2ccccc2-c2ccc(N(c3ccccc3)c3ccc4c(c3)-c3cc5ccccc5cc3C4(C)C)cc21 ; CC1(C)c2ccc(N(c3ccccc3)c3ccc4c(c3)C(c3ccccc3)(c3ccccc3)c3ccccc3-4)cc2-c2cc3ccccc3cc21]

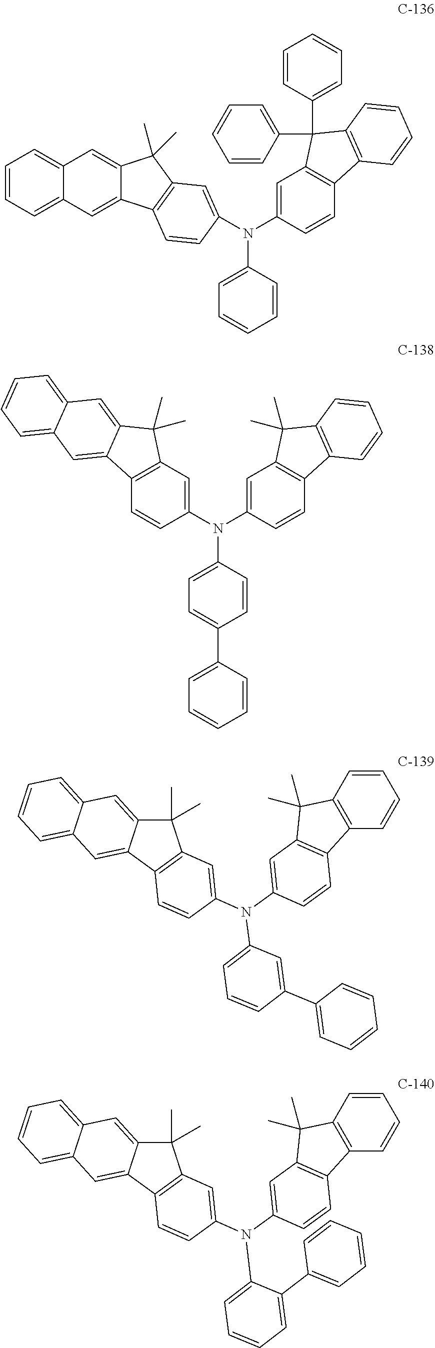

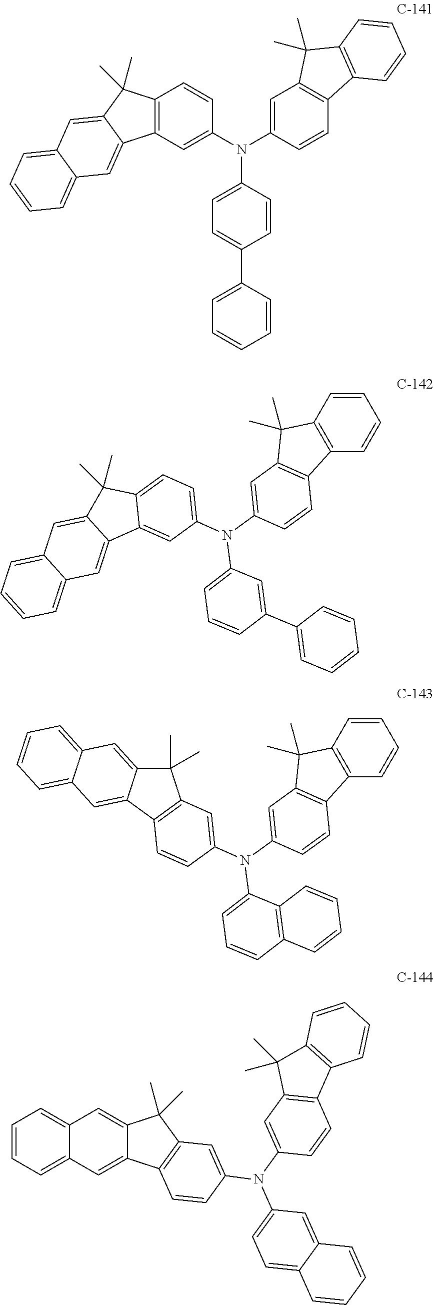 [SMILES: CC1(C)c2ccccc2-c2ccc(N(c3ccc(-c4ccccc4)cc3)c3ccc4c(c3)-c3cc5ccccc5cc3C4(C)C)cc21 ; CC1(C)c2ccccc2-c2ccc(N(c3cccc(-c4ccccc4)c3)c3ccc4c(c3)-c3cc5ccccc5cc3C4(C)C)cc21 ; CC1(C)c2ccccc2-c2ccc(N(c3ccc4c(c3)C(C)(C)c3cc5ccccc5cc3-4)c3cccc4ccccc34)cc21 ; CC1(C)c2ccccc2-c2ccc(N(c3ccc4c(c3)C(C)(C)c3cc5ccccc5cc3-4)c3ccc4ccccc4c3)cc21]

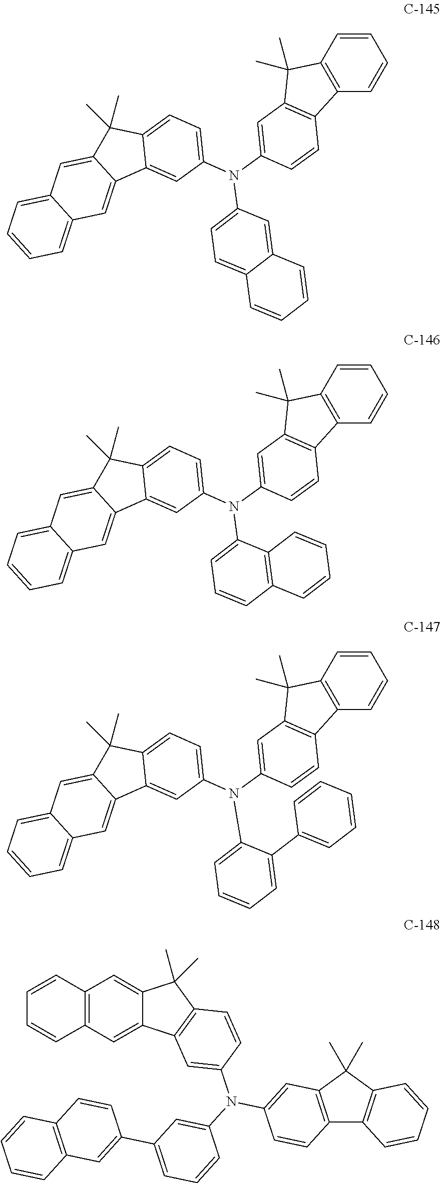 [SMILES: CC1(C)c2ccccc2-c2ccc(N(c3ccc4c(c3)-c3cc5ccccc5cc3C4(C)C)c3ccc4ccccc4c3)cc21 ; CC1(C)c2ccccc2-c2ccc(N(c3ccc4c(c3)-c3cc5ccccc5cc3C4(C)C)c3cccc4ccccc34)cc21 ; CC1(C)c2ccccc2-c2ccc(N(c3ccc4c(c3)-c3cc5ccccc5cc3C4(C)C)c3ccccc3-c3ccccc3)cc21 ; CC1(C)c2ccccc2-c2ccc(N(c3cccc(-c4ccc5ccccc5c4)c3)c3ccc4c(c3)-c3cc5ccccc5cc3C4(C)C)cc21]

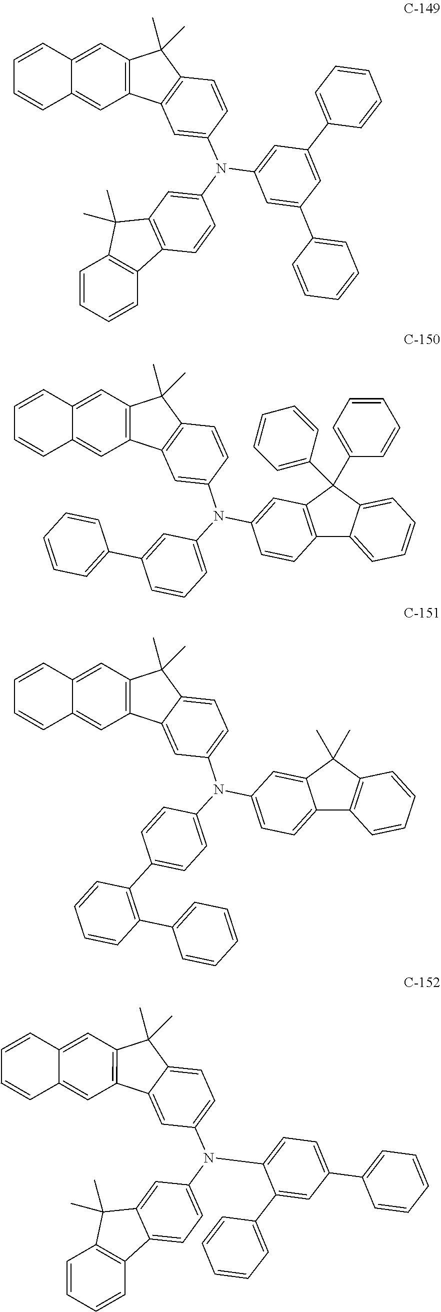 [SMILES: CC1(C)c2ccccc2-c2ccc(N(c3cc(-c4ccccc4)cc(-c4ccccc4)c3)c3ccc4c(c3)-c3cc5ccccc5cc3C4(C)C)cc21 ; CC1(C)c2ccc(N(c3cccc(-c4ccccc4)c3)c3ccc4c(c3)C(c3ccccc3)(c3ccccc3)c3ccccc3-4)cc2-c2cc3ccccc3cc21 ; CC1(C)c2ccccc2-c2ccc(N(c3ccc(-c4ccccc4-c4ccccc4)cc3)c3ccc4c(c3)-c3cc5ccccc5cc3C4(C)C)cc21 ; CC1(C)c2ccccc2-c2ccc(N(c3ccc4c(c3)-c3cc5ccccc5cc3C4(C)C)c3ccc(-c4ccccc4)cc3-c3ccccc3)cc21]

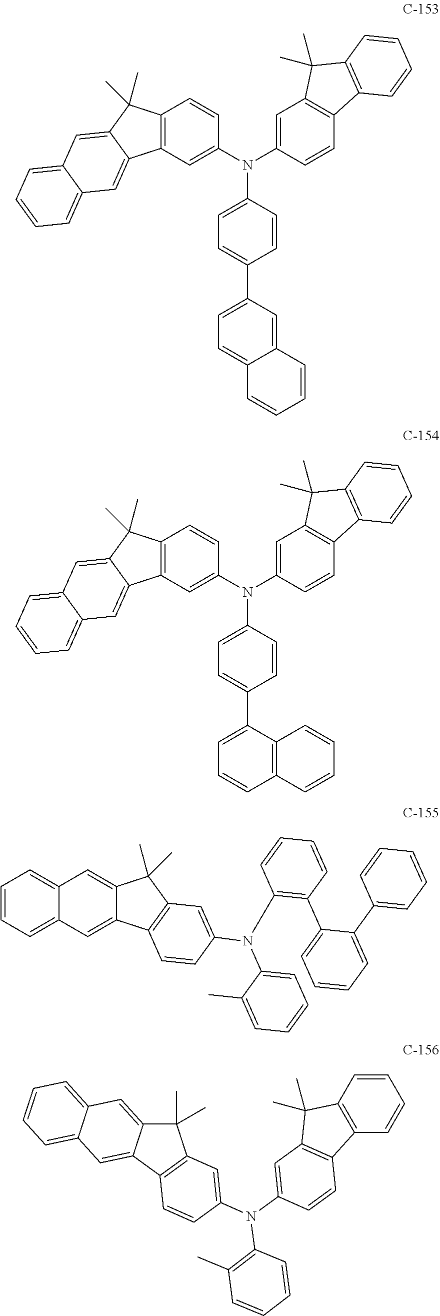 [SMILES: CC1(C)c2ccccc2-c2ccc(N(c3ccc(-c4ccc5ccccc5c4)cc3)c3ccc4c(c3)-c3cc5ccccc5cc3C4(C)C)cc21 ; CC1(C)c2ccccc2-c2ccc(N(c3ccc(-c4cccc5ccccc45)cc3)c3ccc4c(c3)-c3cc5ccccc5cc3C4(C)C)cc21 ; Cc1ccccc1N(c1ccc2c(c1)C(C)(C)c1cc3ccccc3cc1-2)c1ccccc1-c1ccccc1-c1ccccc1 ; Cc1ccccc1N(c1ccc2c(c1)C(C)(C)c1ccccc1-2)c1ccc2c(c1)C(C)(C)c1cc3ccccc3cc1-2]

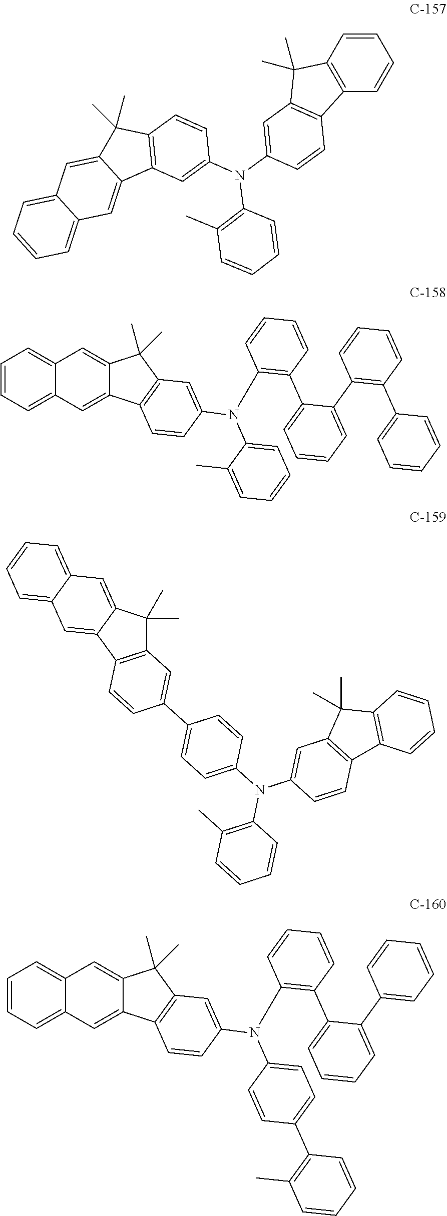

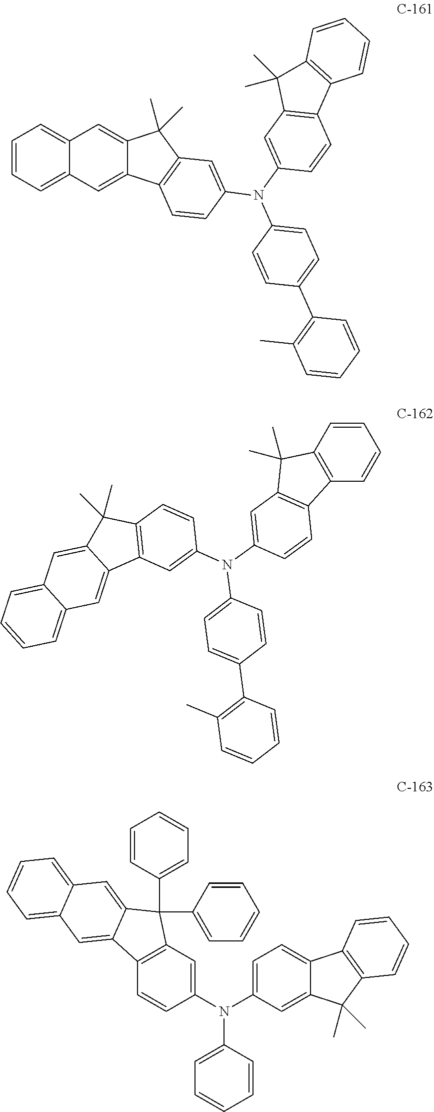

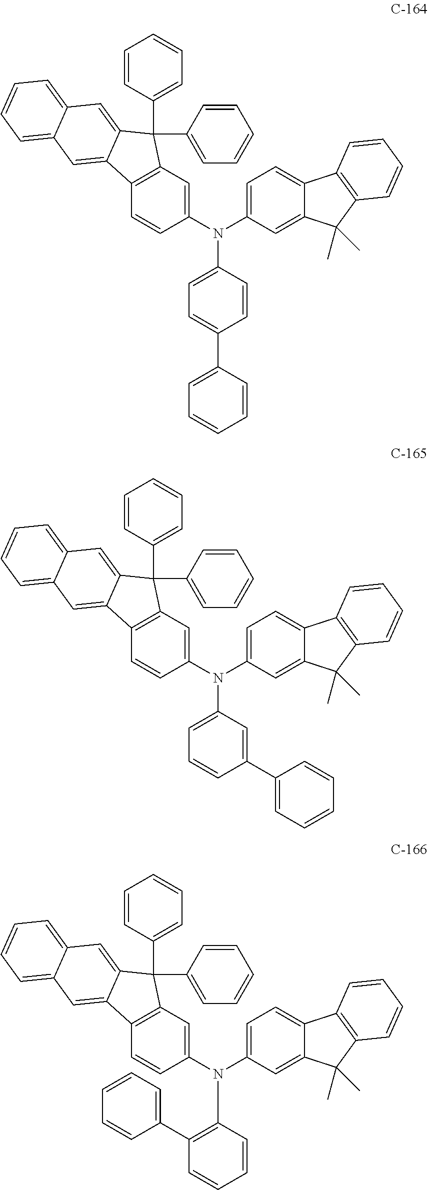 [SMILES: CC1(C)c2ccccc2-c2ccc(N(c3ccc(-c4ccccc4)cc3)c3ccc4c(c3)C(c3ccccc3)(c3ccccc3)c3cc5ccccc5cc3-4)cc21 ; CC1(C)c2ccccc2-c2ccc(N(c3cccc(-c4ccccc4)c3)c3ccc4c(c3)C(c3ccccc3)(c3ccccc3)c3cc5ccccc5cc3-4)cc21 ; CC1(C)c2ccccc2-c2ccc(N(c3ccc4c(c3)C(c3ccccc3)(c3ccccc3)c3cc5ccccc5cc3-4)c3ccccc3-c3ccccc3)cc21]

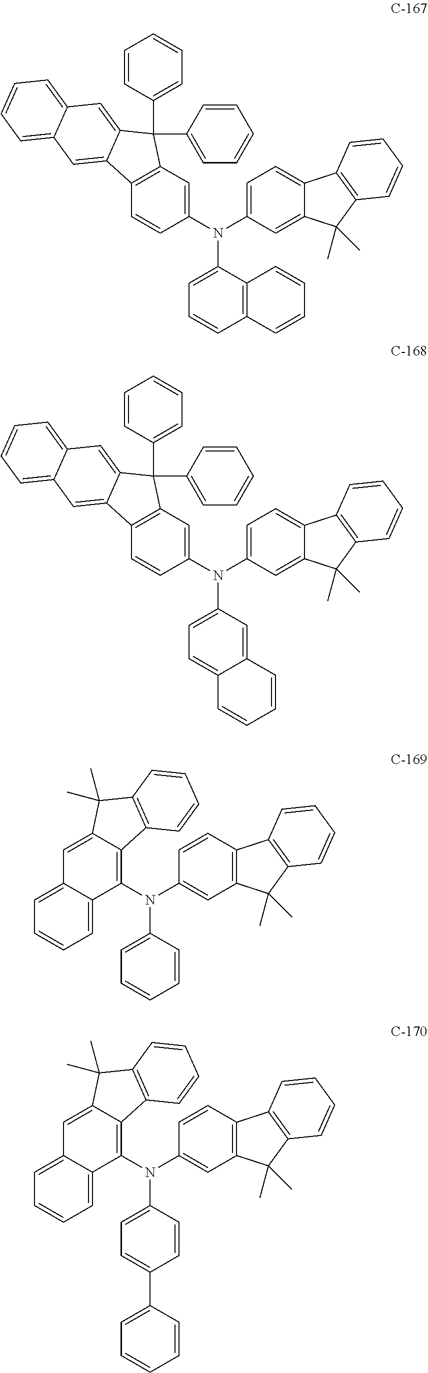

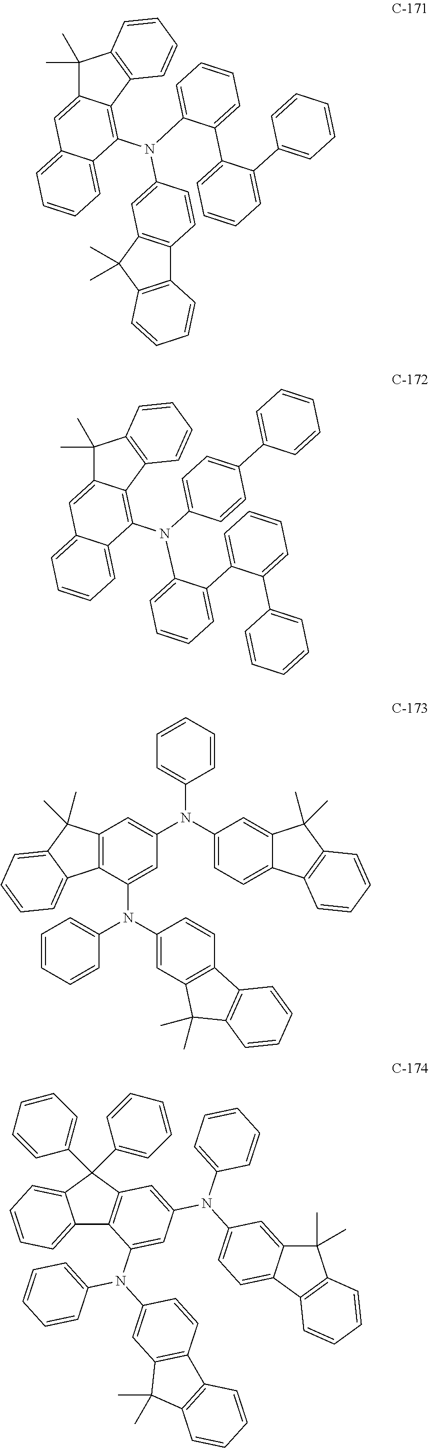 [SMILES: CC1(C)c2ccccc2-c2ccc(N(c3ccccc3-c3ccccc3-c3ccccc3)c3c4c(cc5ccccc35)C(C)(C)c3ccccc3-4)cc21 ; CC1(C)c2ccccc2-c2c1cc1ccccc1c2N(c1ccc(-c2ccccc2)cc1)c1ccccc1-c1ccccc1-c1ccccc1 ; CC1(C)c2ccccc2-c2ccc(N(c3ccccc3)c3cc(N(c4ccccc4)c4ccc5c(c4)C(C)(C)c4ccccc4-5)c4c(c3)C(C)(C)c3ccccc3-4)cc21 ; CC1(C)c2ccccc2-c2ccc(N(c3ccccc3)c3cc(N(c4ccccc4)c4ccc5c(c4)C(C)(C)c4ccccc4-5)c4c(c3)C(c3ccccc3)(c3ccccc3)c3ccccc3-4)cc21]

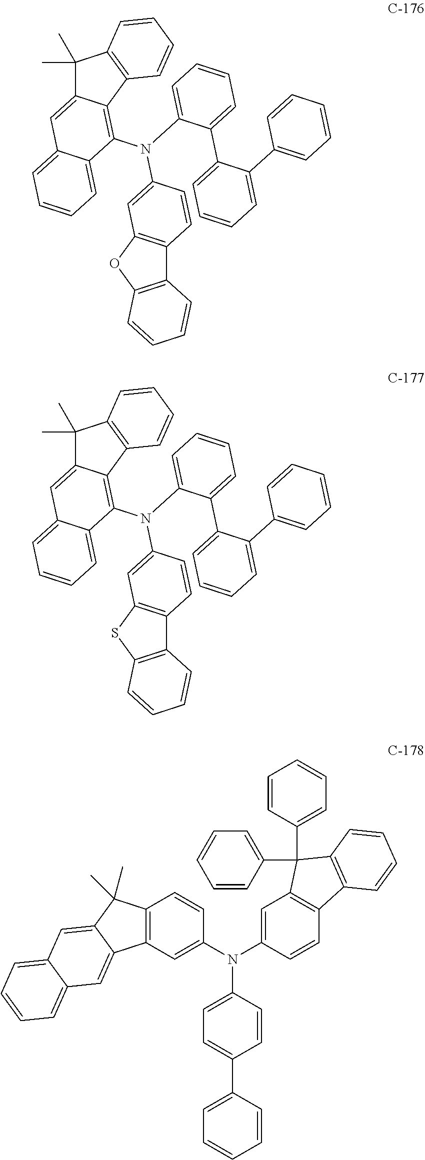

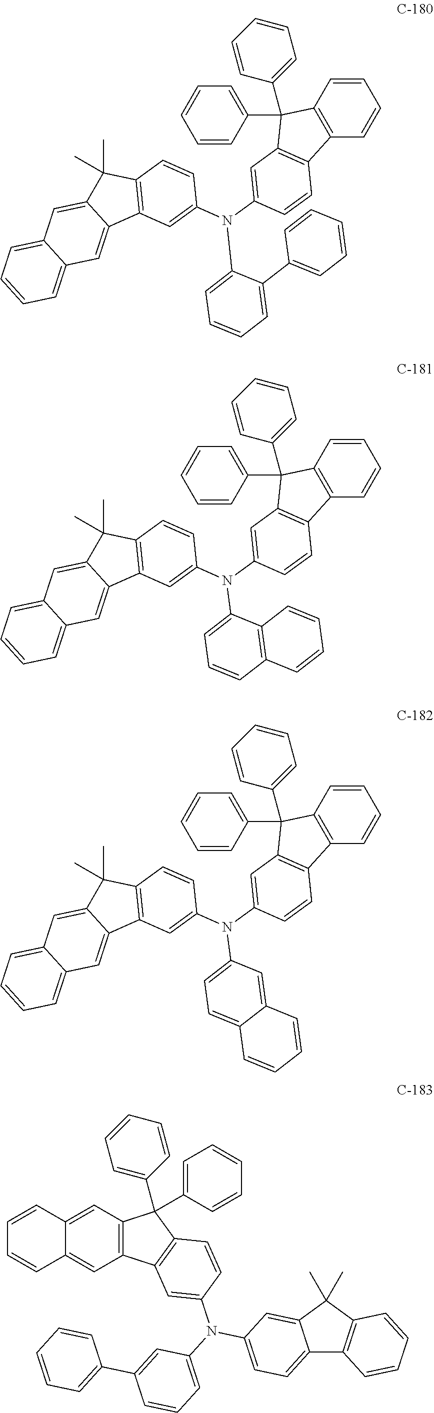

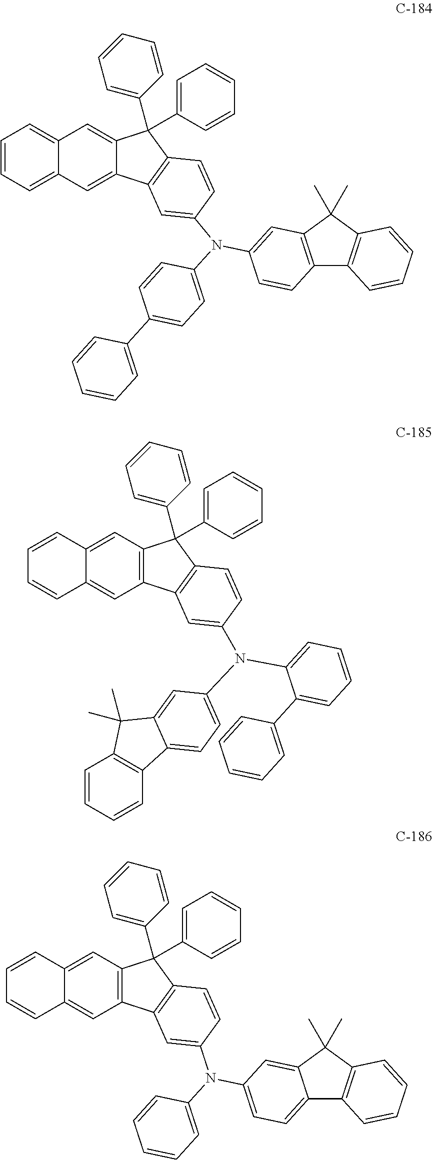

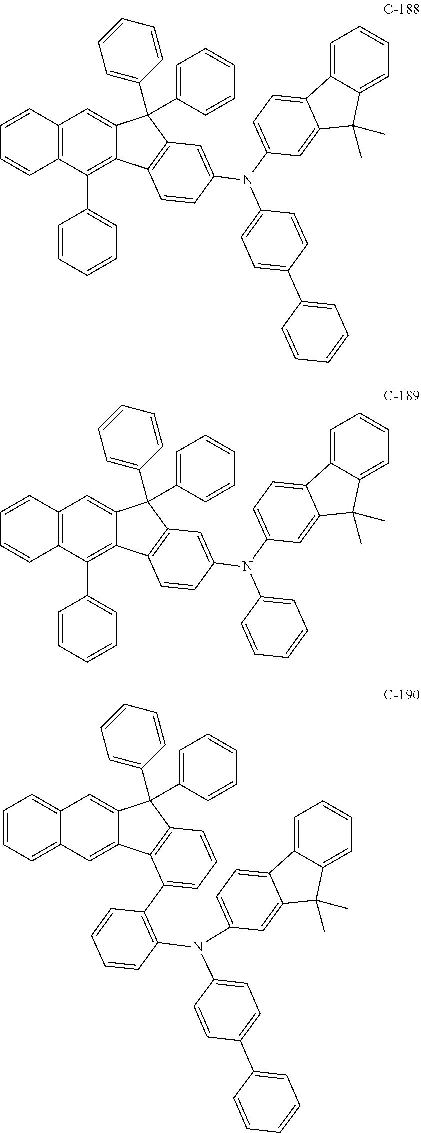 [SMILES: CC1(C)c2ccccc2-c2ccc(N(c3ccc(-c4ccccc4)cc3)c3ccc4c(c3)C(c3ccccc3)(c3ccccc3)c3cc5ccccc5c(-c5ccccc5)c3-4)cc21 ; CC1(C)c2ccccc2-c2ccc(N(c3ccccc3)c3ccc4c(c3)C(c3ccccc3)(c3ccccc3)c3cc5ccccc5c(-c5ccccc5)c3-4)cc21 ; CC1(C)c2ccccc2-c2ccc(N(c3ccc(-c4ccccc4)cc3)c3ccccc3-c3cccc4c3-c3cc5ccccc5cc3C4(c3ccccc3)c3ccccc3)cc21]

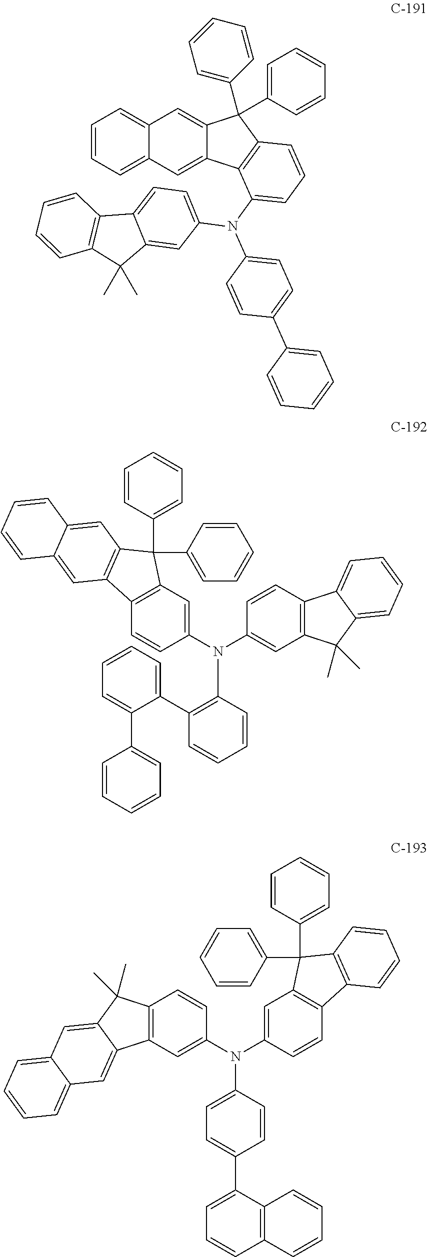 [SMILES: CC1(C)c2ccccc2-c2ccc(N(c3ccc(-c4ccccc4)cc3)c3cccc4c3-c3cc5ccccc5cc3C4(c3ccccc3)c3ccccc3)cc21 ; CC1(C)c2ccccc2-c2ccc(N(c3ccc4c(c3)C(c3ccccc3)(c3ccccc3)c3cc5ccccc5cc3-4)c3ccccc3-c3ccccc3-c3ccccc3)cc21 ; CC1(C)c2ccc(N(c3ccc(-c4cccc5ccccc45)cc3)c3ccc4c(c3)C(c3ccccc3)(c3ccccc3)c3ccccc3-4)cc2-c2cc3ccccc3cc21]

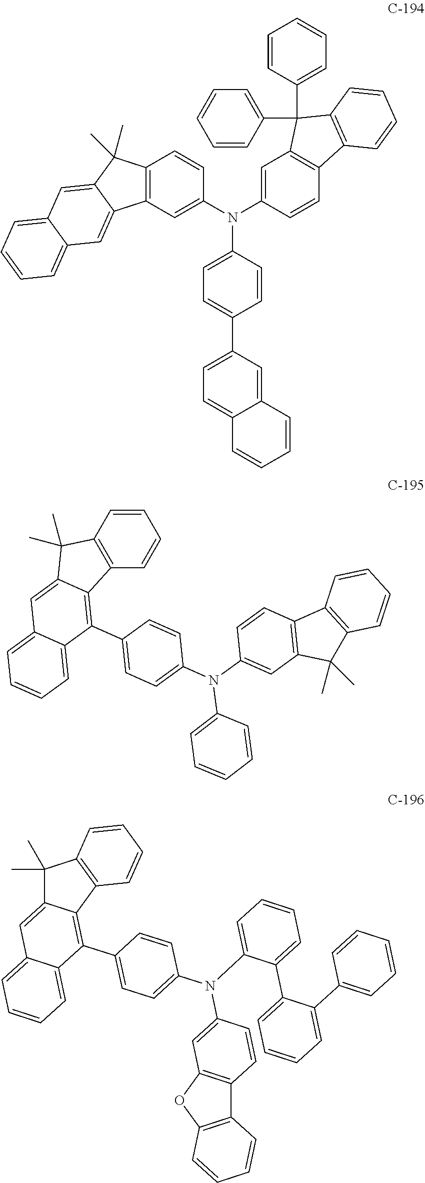 [SMILES: CC1(C)c2ccc(N(c3ccc(-c4ccc5ccccc5c4)cc3)c3ccc4c(c3)C(c3ccccc3)(c3ccccc3)c3ccccc3-4)cc2-c2cc3ccccc3cc21 ; CC1(C)c2ccccc2-c2ccc(N(c3ccccc3)c3ccc(-c4c5c(cc6ccccc46)C(C)(C)c4ccccc4-5)cc3)cc21 ; CC1(C)c2ccccc2-c2c1cc1ccccc1c2-c1ccc(N(c2ccc3c(c2)oc2ccccc23)c2ccccc2-c2ccccc2-c2ccccc2)cc1]

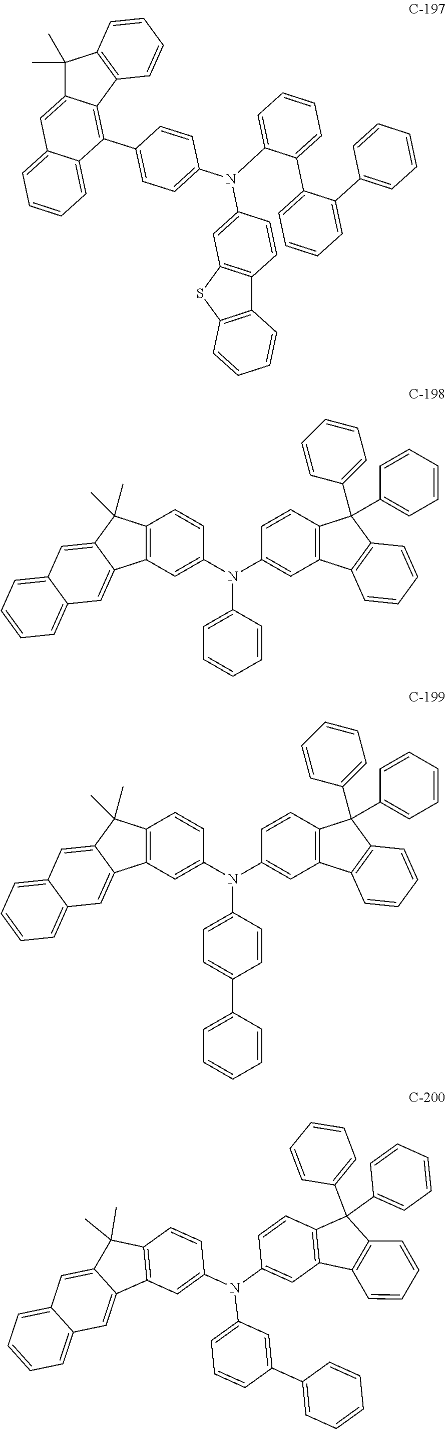 [SMILES: CC1(C)c2ccccc2-c2c1cc1ccccc1c2-c1ccc(N(c2ccc3c(c2)sc2ccccc23)c2ccccc2-c2ccccc2-c2ccccc2)cc1 ; CC1(C)c2ccc(N(c3ccccc3)c3ccc4c(c3)-c3ccccc3C4(c3ccccc3)c3ccccc3)cc2-c2cc3ccccc3cc21 ; CC1(C)c2ccc(N(c3ccc(-c4ccccc4)cc3)c3ccc4c(c3)-c3ccccc3C4(c3ccccc3)c3ccccc3)cc2-c2cc3ccccc3cc21 ; CC1(C)c2ccc(N(c3cccc(-c4ccccc4)c3)c3ccc4c(c3)-c3ccccc3C4(c3ccccc3)c3ccccc3)cc2-c2cc3ccccc3cc21]

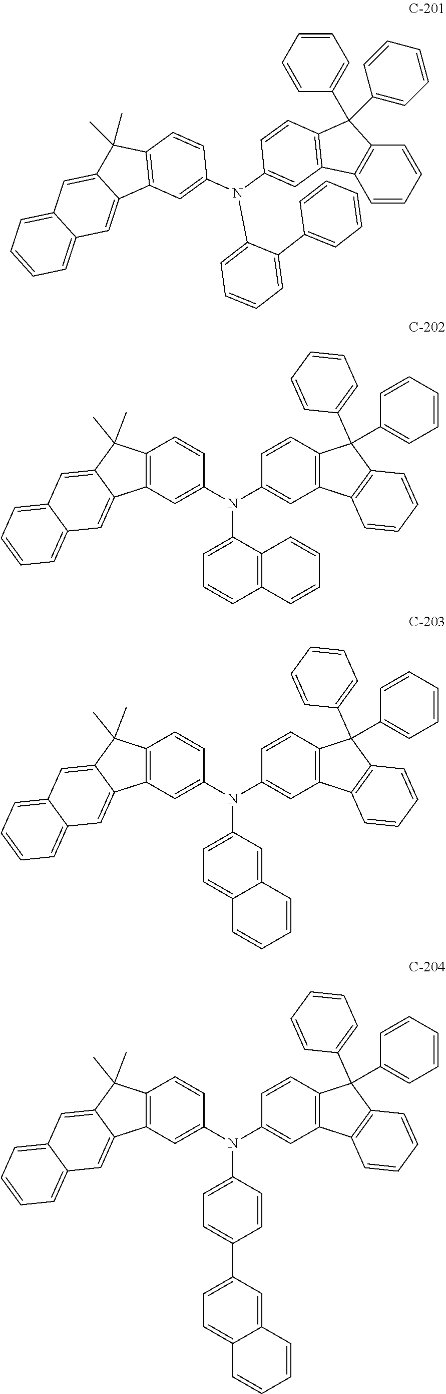 [SMILES: CC1(C)c2ccc(N(c3ccc4c(c3)-c3ccccc3C4(c3ccccc3)c3ccccc3)c3ccccc3-c3ccccc3)cc2-c2cc3ccccc3cc21 ; CC1(C)c2ccc(N(c3ccc4c(c3)-c3ccccc3C4(c3ccccc3)c3ccccc3)c3cccc4ccccc34)cc2-c2cc3ccccc3cc21 ; CC1(C)c2ccc(N(c3ccc4c(c3)-c3ccccc3C4(c3ccccc3)c3ccccc3)c3ccc4ccccc4c3)cc2-c2cc3ccccc3cc21 ; CC1(C)c2ccc(N(c3ccc(-c4ccc5ccccc5c4)cc3)c3ccc4c(c3)-c3ccccc3C4(c3ccccc3)c3ccccc3)cc2-c2cc3ccccc3cc21]

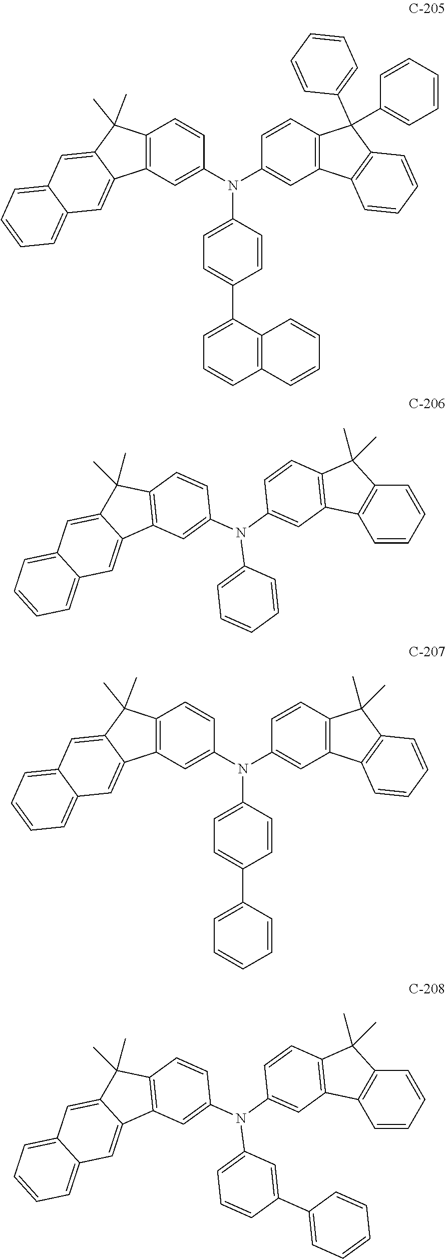 [SMILES: CC1(C)c2ccc(N(c3ccc(-c4cccc5ccccc45)cc3)c3ccc4c(c3)-c3ccccc3C4(c3ccccc3)c3ccccc3)cc2-c2cc3ccccc3cc21 ; CC1(C)c2ccccc2-c2cc(N(c3ccccc3)c3ccc4c(c3)-c3cc5ccccc5cc3C4(C)C)ccc21 ; CC1(C)c2ccccc2-c2cc(N(c3ccc(-c4ccccc4)cc3)c3ccc4c(c3)-c3cc5ccccc5cc3C4(C)C)ccc21 ; CC1(C)c2ccccc2-c2cc(N(c3cccc(-c4ccccc4)c3)c3ccc4c(c3)-c3cc5ccccc5cc3C4(C)C)ccc21]

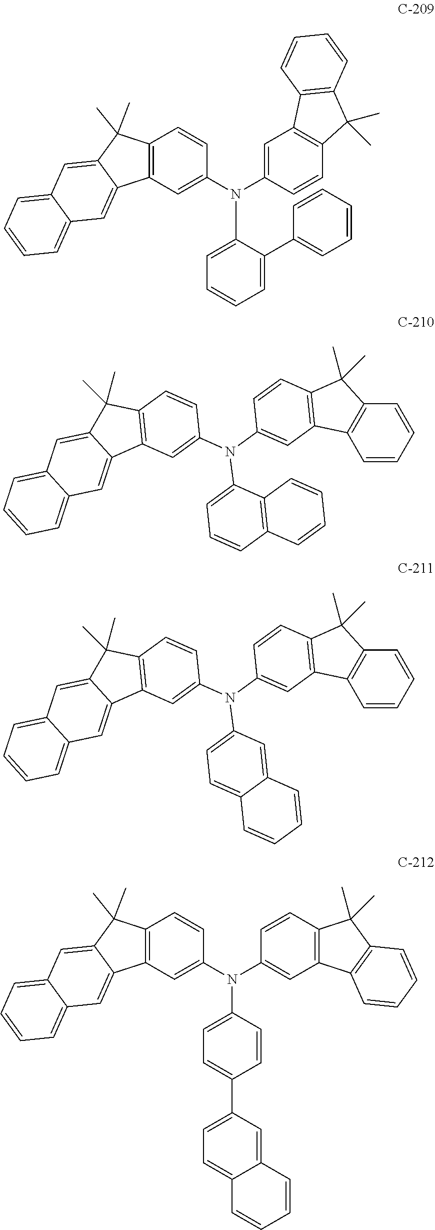 [SMILES: CC1(C)c2ccccc2-c2cc(N(c3ccc4c(c3)-c3cc5ccccc5cc3C4(C)C)c3ccccc3-c3ccccc3)ccc21 ; CC1(C)c2ccccc2-c2cc(N(c3ccc4c(c3)-c3cc5ccccc5cc3C4(C)C)c3cccc4ccccc34)ccc21 ; CC1(C)c2ccccc2-c2cc(N(c3ccc4c(c3)-c3cc5ccccc5cc3C4(C)C)c3ccc4ccccc4c3)ccc21 ; CC1(C)c2ccccc2-c2cc(N(c3ccc(-c4ccc5ccccc5c4)cc3)c3ccc4c(c3)-c3cc5ccccc5cc3C4(C)C)ccc21]

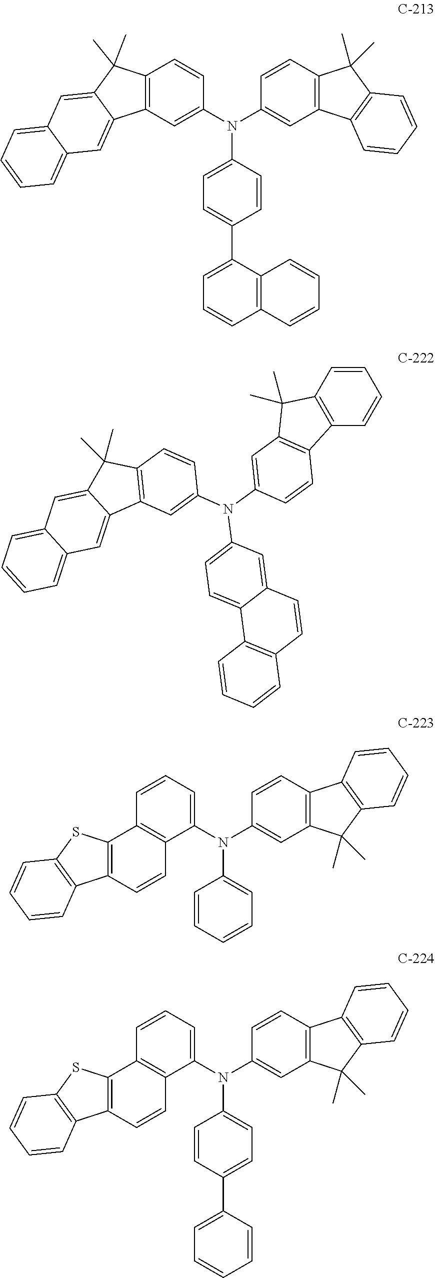 [SMILES: CC1(C)c2ccccc2-c2cc(N(c3ccc(-c4cccc5ccccc45)cc3)c3ccc4c(c3)-c3cc5ccccc5cc3C4(C)C)ccc21 ; CC1(C)c2ccccc2-c2ccc(N(c3ccc4c(c3)-c3cc5ccccc5cc3C4(C)C)c3ccc4c(ccc5ccccc54)c3)cc21 ; CC1(C)c2ccccc2-c2ccc(N(c3ccccc3)c3cccc4c3ccc3c5ccccc5sc43)cc21 ; CC1(C)c2ccccc2-c2ccc(N(c3ccc(-c4ccccc4)cc3)c3cccc4c3ccc3c5ccccc5sc43)cc21]

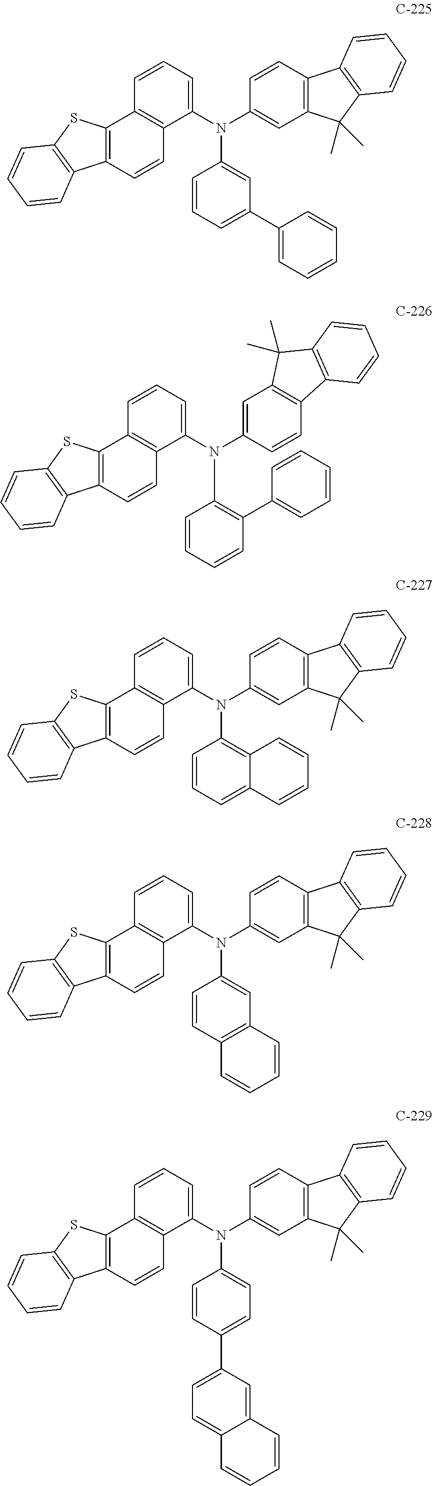 [SMILES: CC1(C)c2ccccc2-c2ccc(N(c3cccc(-c4ccccc4)c3)c3cccc4c3ccc3c5ccccc5sc43)cc21 ; CC1(C)c2ccccc2-c2ccc(N(c3ccccc3-c3ccccc3)c3cccc4c3ccc3c5ccccc5sc43)cc21 ; CC1(C)c2ccccc2-c2ccc(N(c3cccc4ccccc34)c3cccc4c3ccc3c5ccccc5sc43)cc21 ; CC1(C)c2ccccc2-c2ccc(N(c3ccc4ccccc4c3)c3cccc4c3ccc3c5ccccc5sc43)cc21 ; CC1(C)c2ccccc2-c2ccc(N(c3ccc(-c4ccc5ccccc5c4)cc3)c3cccc4c3ccc3c5ccccc5sc43)cc21]

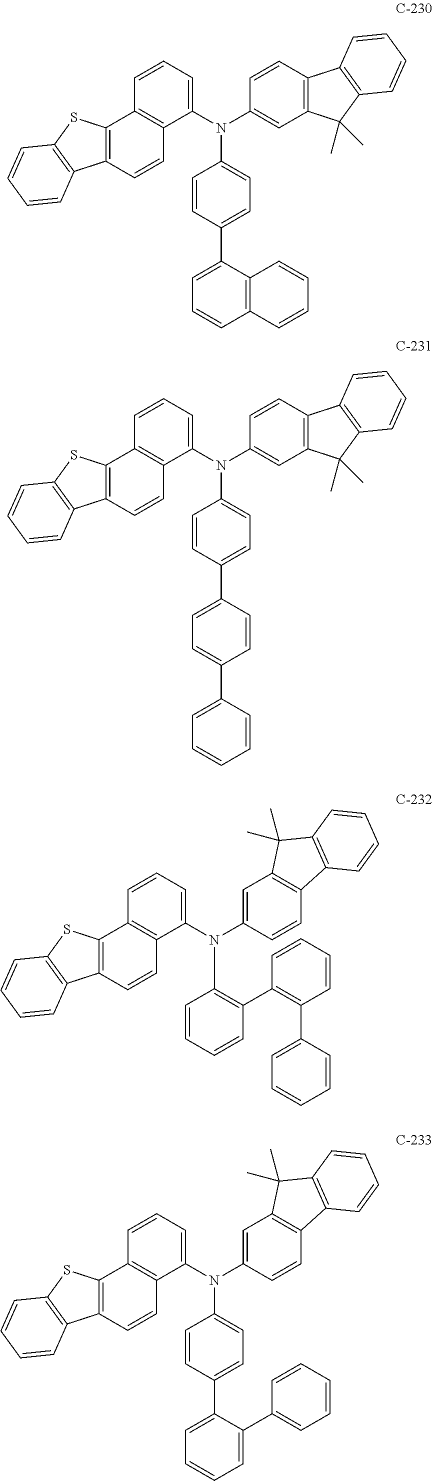 [SMILES: CC1(C)c2ccccc2-c2ccc(N(c3ccc(-c4cccc5ccccc45)cc3)c3cccc4c3ccc3c5ccccc5sc43)cc21 ; CC1(C)c2ccccc2-c2ccc(N(c3ccc(-c4ccc(-c5ccccc5)cc4)cc3)c3cccc4c3ccc3c5ccccc5sc43)cc21 ; CC1(C)c2ccccc2-c2ccc(N(c3ccccc3-c3ccccc3-c3ccccc3)c3cccc4c3ccc3c5ccccc5sc43)cc21 ; CC1(C)c2ccccc2-c2ccc(N(c3ccc(-c4ccccc4-c4ccccc4)cc3)c3cccc4c3ccc3c5ccccc5sc43)cc21]

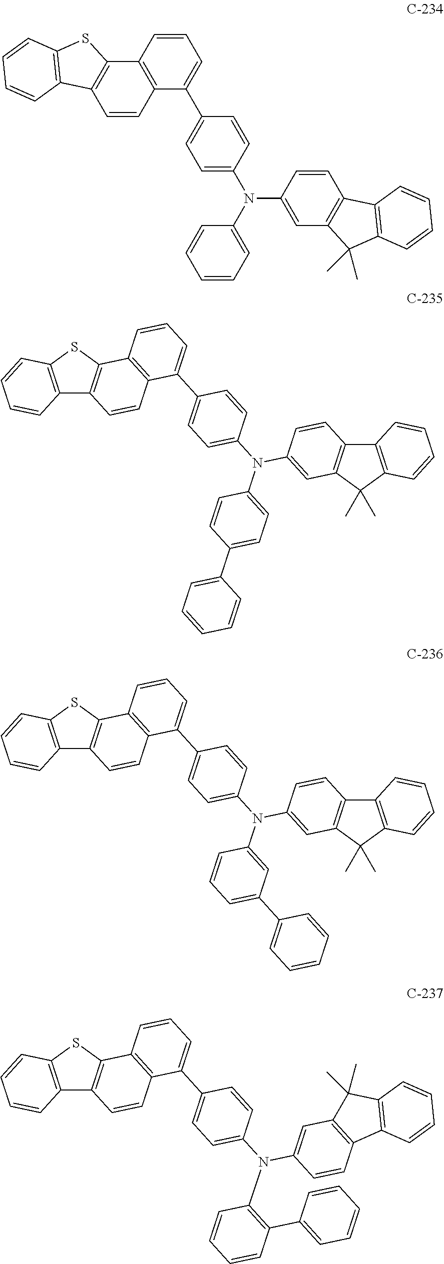 [SMILES: CC1(C)c2ccccc2-c2ccc(N(c3ccccc3)c3ccc(-c4cccc5c4ccc4c6ccccc6sc54)cc3)cc21 ; CC1(C)c2ccccc2-c2ccc(N(c3ccc(-c4ccccc4)cc3)c3ccc(-c4cccc5c4ccc4c6ccccc6sc54)cc3)cc21 ; CC1(C)c2ccccc2-c2ccc(N(c3ccc(-c4cccc5c4ccc4c6ccccc6sc54)cc3)c3cccc(-c4ccccc4)c3)cc21 ; CC1(C)c2ccccc2-c2ccc(N(c3ccc(-c4cccc5c4ccc4c6ccccc6sc54)cc3)c3ccccc3-c3ccccc3)cc21]

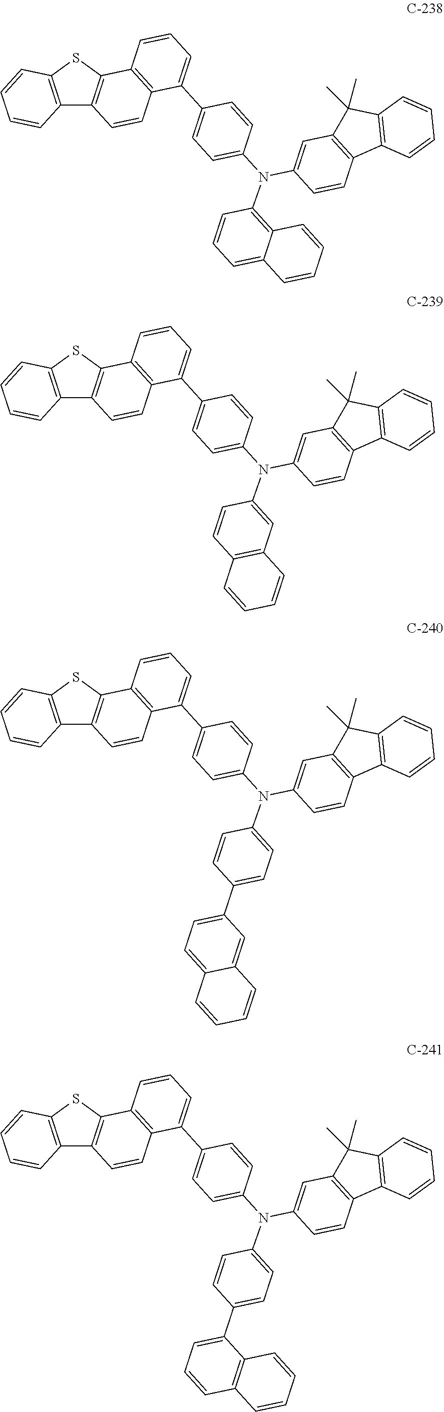 [SMILES: CC1(C)c2ccccc2-c2ccc(N(c3ccc(-c4cccc5c4ccc4c6ccccc6sc54)cc3)c3cccc4ccccc34)cc21 ; CC1(C)c2ccccc2-c2ccc(N(c3ccc(-c4cccc5c4ccc4c6ccccc6sc54)cc3)c3ccc4ccccc4c3)cc21 ; CC1(C)c2ccccc2-c2ccc(N(c3ccc(-c4ccc5ccccc5c4)cc3)c3ccc(-c4cccc5c4ccc4c6ccccc6sc54)cc3)cc21 ; CC1(C)c2ccccc2-c2ccc(N(c3ccc(-c4cccc5ccccc45)cc3)c3ccc(-c4cccc5c4ccc4c6ccccc6sc54)cc3)cc21]

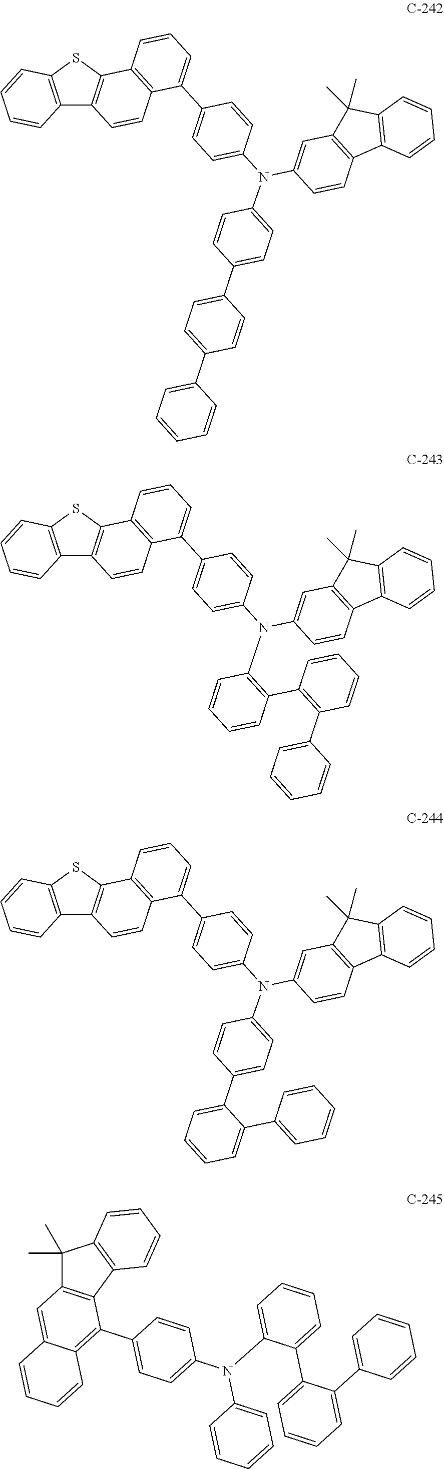 [SMILES: CC1(C)c2ccccc2-c2ccc(N(c3ccc(-c4ccc(-c5ccccc5)cc4)cc3)c3ccc(-c4cccc5c4ccc4c6ccccc6sc54)cc3)cc21 ; CC1(C)c2ccccc2-c2ccc(N(c3ccc(-c4cccc5c4ccc4c6ccccc6sc54)cc3)c3ccccc3-c3ccccc3-c3ccccc3)cc21 ; CC1(C)c2ccccc2-c2ccc(N(c3ccc(-c4ccccc4-c4ccccc4)cc3)c3ccc(-c4cccc5c4ccc4c6ccccc6sc54)cc3)cc21 ; CC1(C)c2ccccc2-c2c1cc1ccccc1c2-c1ccc(N(c2ccccc2)c2ccccc2-c2ccccc2-c2ccccc2)cc1]

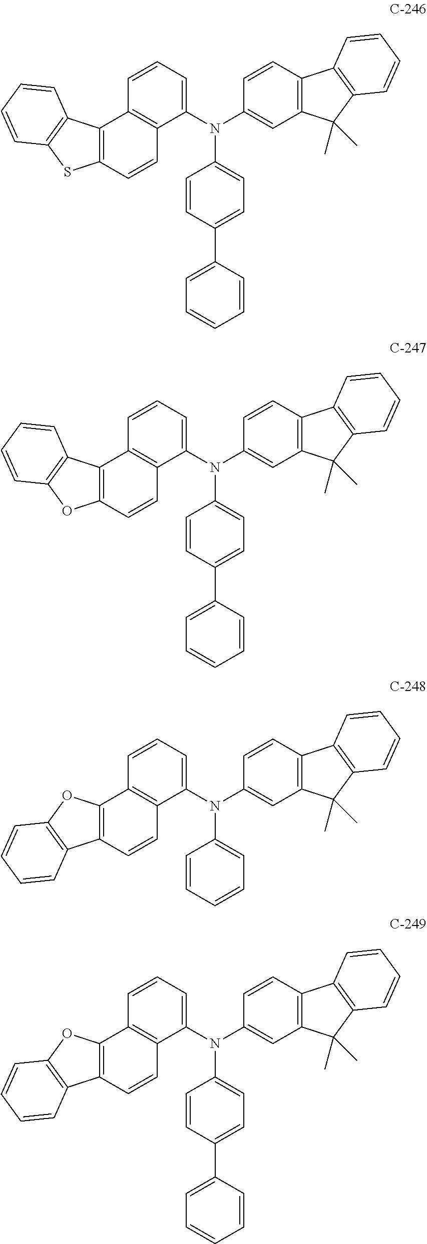 [SMILES: CC1(C)c2ccccc2-c2ccc(N(c3ccc(-c4ccccc4)cc3)c3cccc4c3ccc3sc5ccccc5c34)cc21 ; CC1(C)c2ccccc2-c2ccc(N(c3ccc(-c4ccccc4)cc3)c3cccc4c3ccc3oc5ccccc5c34)cc21 ; CC1(C)c2ccccc2-c2ccc(N(c3ccccc3)c3cccc4c3ccc3c5ccccc5oc43)cc21 ; CC1(C)c2ccccc2-c2ccc(N(c3ccc(-c4ccccc4)cc3)c3cccc4c3ccc3c5ccccc5oc43)cc21]

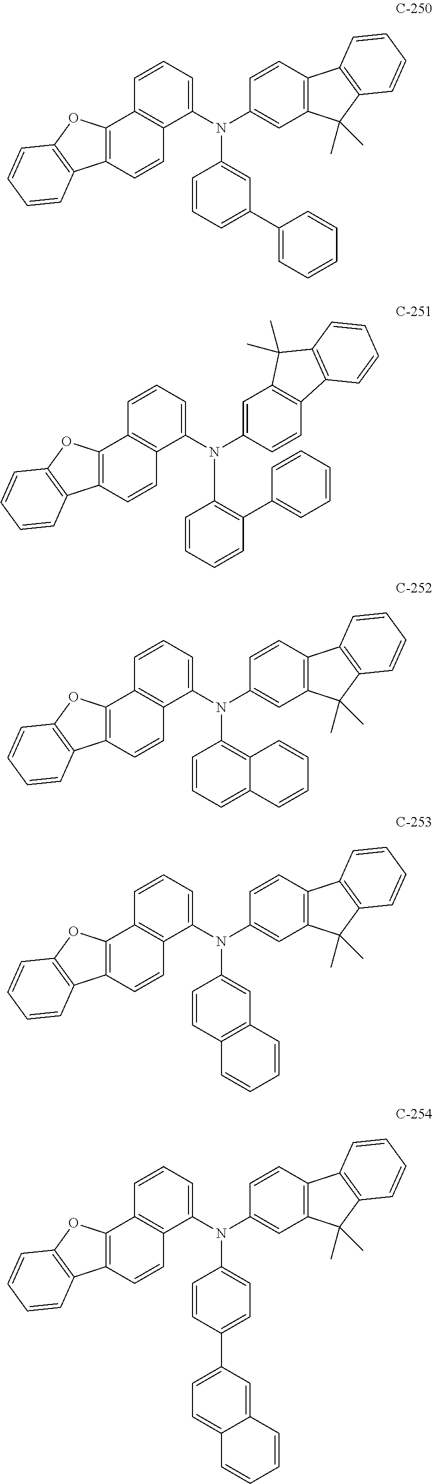 [SMILES: CC1(C)c2ccccc2-c2ccc(N(c3cccc(-c4ccccc4)c3)c3cccc4c3ccc3c5ccccc5oc43)cc21 ; CC1(C)c2ccccc2-c2ccc(N(c3ccccc3-c3ccccc3)c3cccc4c3ccc3c5ccccc5oc43)cc21 ; CC1(C)c2ccccc2-c2ccc(N(c3cccc4ccccc34)c3cccc4c3ccc3c5ccccc5oc43)cc21 ; CC1(C)c2ccccc2-c2ccc(N(c3ccc4ccccc4c3)c3cccc4c3ccc3c5ccccc5oc43)cc21 ; CC1(C)c2ccccc2-c2ccc(N(c3ccc(-c4ccc5ccccc5c4)cc3)c3cccc4c3ccc3c5ccccc5oc43)cc21]

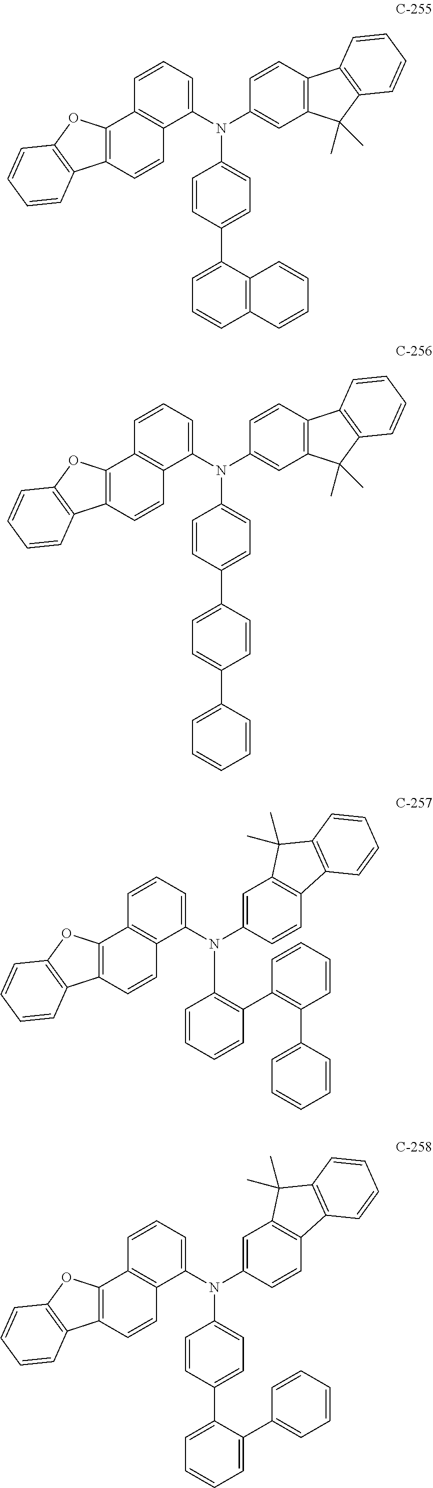 [SMILES: CC1(C)c2ccccc2-c2ccc(N(c3ccc(-c4cccc5ccccc45)cc3)c3cccc4c3ccc3c5ccccc5oc43)cc21 ; CC1(C)c2ccccc2-c2ccc(N(c3ccc(-c4ccc(-c5ccccc5)cc4)cc3)c3cccc4c3ccc3c5ccccc5oc43)cc21 ; CC1(C)c2ccccc2-c2ccc(N(c3ccccc3-c3ccccc3-c3ccccc3)c3cccc4c3ccc3c5ccccc5oc43)cc21 ; CC1(C)c2ccccc2-c2ccc(N(c3ccc(-c4ccccc4-c4ccccc4)cc3)c3cccc4c3ccc3c5ccccc5oc43)cc21]

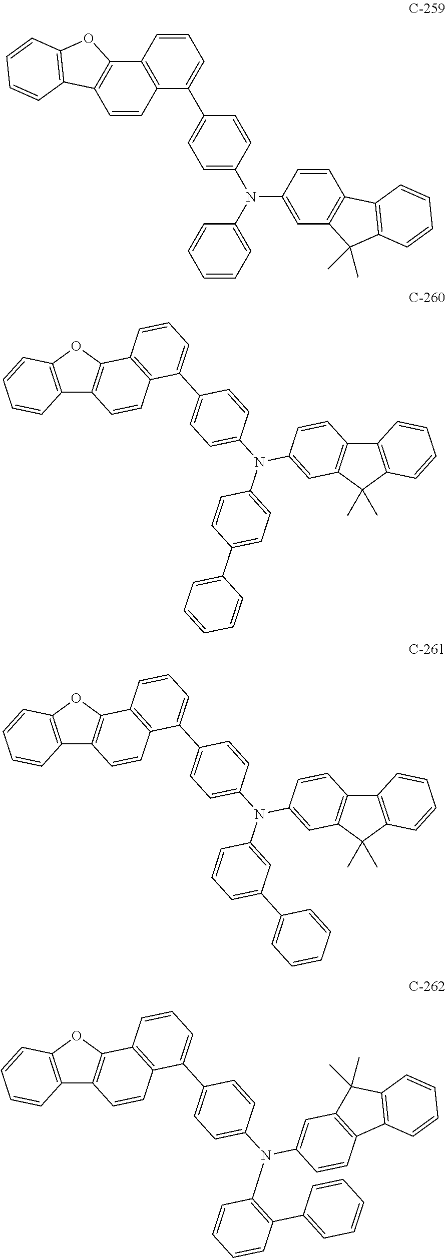 [SMILES: CC1(C)c2ccccc2-c2ccc(N(c3ccccc3)c3ccc(-c4cccc5c4ccc4c6ccccc6oc54)cc3)cc21 ; CC1(C)c2ccccc2-c2ccc(N(c3ccc(-c4ccccc4)cc3)c3ccc(-c4cccc5c4ccc4c6ccccc6oc54)cc3)cc21 ; CC1(C)c2ccccc2-c2ccc(N(c3ccc(-c4cccc5c4ccc4c6ccccc6oc54)cc3)c3cccc(-c4ccccc4)c3)cc21 ; CC1(C)c2ccccc2-c2ccc(N(c3ccc(-c4cccc5c4ccc4c6ccccc6oc54)cc3)c3ccccc3-c3ccccc3)cc21]

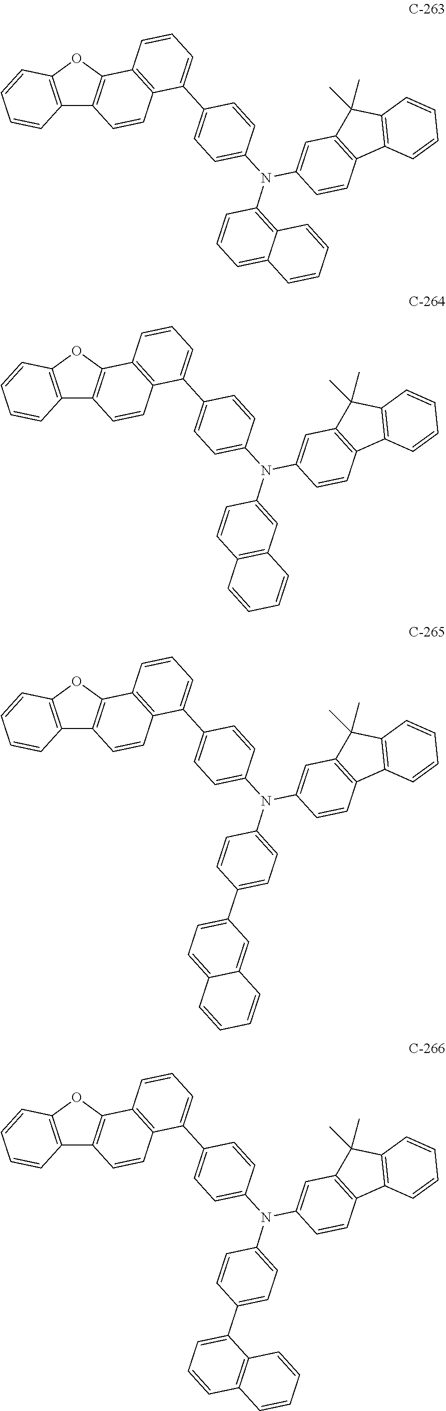 [SMILES: CC1(C)c2ccccc2-c2ccc(N(c3ccc(-c4cccc5c4ccc4c6ccccc6oc54)cc3)c3cccc4ccccc34)cc21 ; CC1(C)c2ccccc2-c2ccc(N(c3ccc(-c4cccc5c4ccc4c6ccccc6oc54)cc3)c3ccc4ccccc4c3)cc21 ; CC1(C)c2ccccc2-c2ccc(N(c3ccc(-c4ccc5ccccc5c4)cc3)c3ccc(-c4cccc5c4ccc4c6ccccc6oc54)cc3)cc21 ; CC1(C)c2ccccc2-c2ccc(N(c3ccc(-c4cccc5ccccc45)cc3)c3ccc(-c4cccc5c4ccc4c6ccccc6oc54)cc3)cc21]

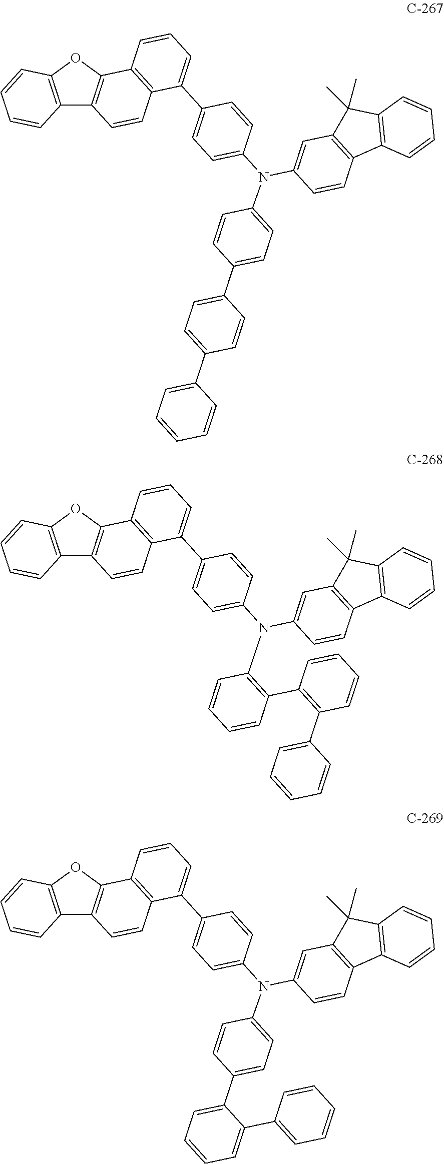 [SMILES: CC1(C)c2ccccc2-c2ccc(N(c3ccc(-c4ccc(-c5ccccc5)cc4)cc3)c3ccc(-c4cccc5c4ccc4c6ccccc6oc54)cc3)cc21 ; CC1(C)c2ccccc2-c2ccc(N(c3ccc(-c4cccc5c4ccc4c6ccccc6oc54)cc3)c3ccccc3-c3ccccc3-c3ccccc3)cc21 ; CC1(C)c2ccccc2-c2ccc(N(c3ccc(-c4ccccc4-c4ccccc4)cc3)c3ccc(-c4cccc5c4ccc4c6ccccc6oc54)cc3)cc21]

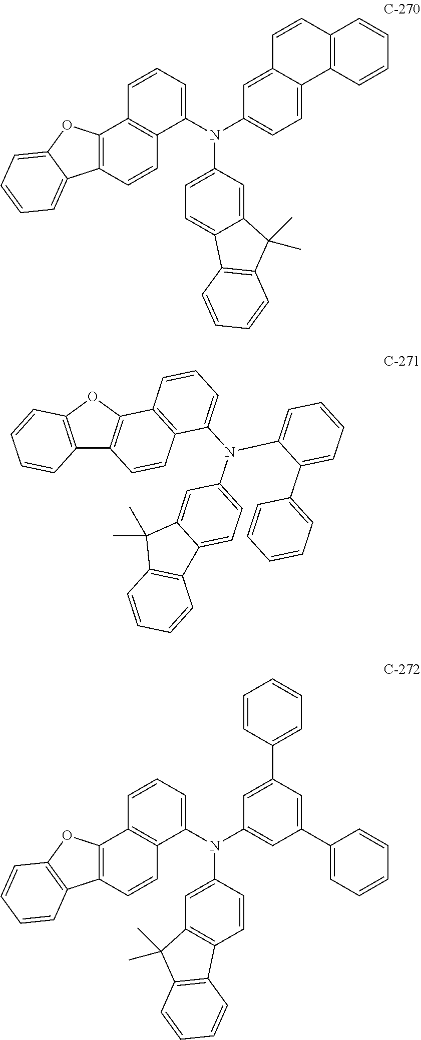 [SMILES: CC1(C)c2ccccc2-c2ccc(N(c3ccc4c(ccc5ccccc54)c3)c3cccc4c3ccc3c5ccccc5oc43)cc21 ; CC1(C)c2ccccc2-c2ccc(N(c3ccccc3-c3ccccc3)c3cccc4c3ccc3c5ccccc5oc43)cc21 ; CC1(C)c2ccccc2-c2ccc(N(c3cc(-c4ccccc4)cc(-c4ccccc4)c3)c3cccc4c3ccc3c5ccccc5oc43)cc21]

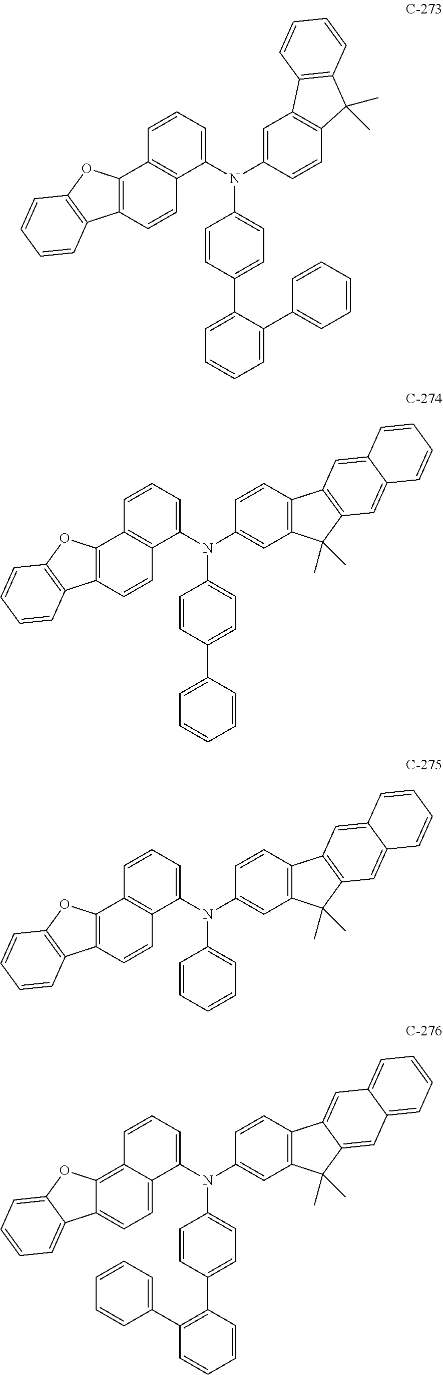 [SMILES: CC1(C)c2ccccc2-c2cc(N(c3ccc(-c4ccccc4-c4ccccc4)cc3)c3cccc4c3ccc3c5ccccc5oc43)ccc21 ; CC1(C)c2cc(N(c3ccc(-c4ccccc4)cc3)c3cccc4c3ccc3c5ccccc5oc43)ccc2-c2cc3ccccc3cc21 ; CC1(C)c2cc(N(c3ccccc3)c3cccc4c3ccc3c5ccccc5oc43)ccc2-c2cc3ccccc3cc21 ; CC1(C)c2cc(N(c3ccc(-c4ccccc4-c4ccccc4)cc3)c3cccc4c3ccc3c5ccccc5oc43)ccc2-c2cc3ccccc3cc21]

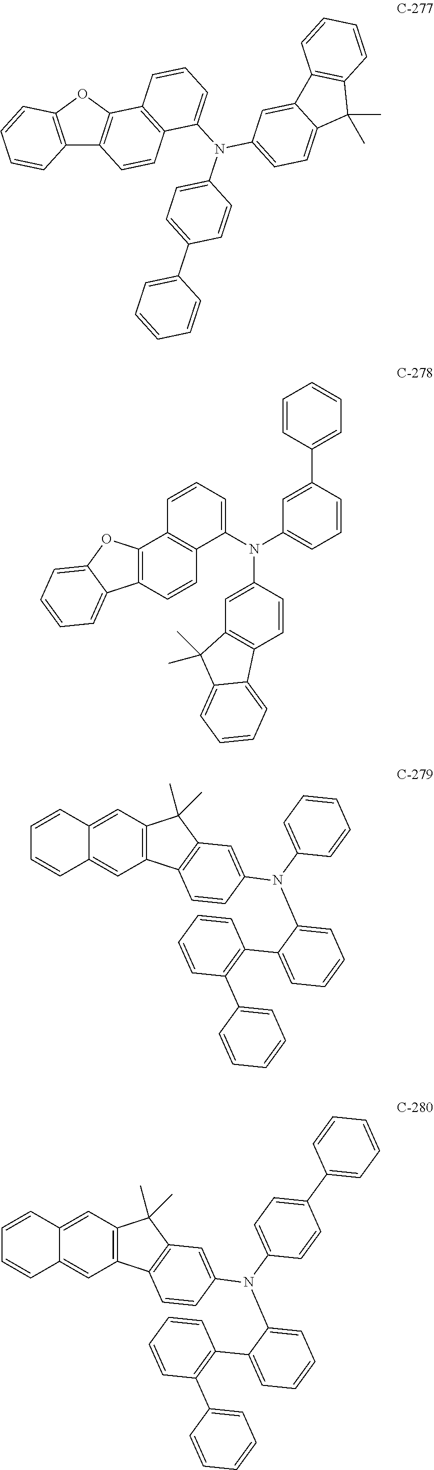 [SMILES: CC1(C)c2ccccc2-c2cc(N(c3ccc(-c4ccccc4)cc3)c3cccc4c3ccc3c5ccccc5oc43)ccc21 ; CC1(C)c2ccccc2-c2ccc(N(c3cccc(-c4ccccc4)c3)c3cccc4c3ccc3c5ccccc5oc43)cc21 ; CC1(C)c2cc(N(c3ccccc3)c3ccccc3-c3ccccc3-c3ccccc3)ccc2-c2cc3ccccc3cc21 ; CC1(C)c2cc(N(c3ccc(-c4ccccc4)cc3)c3ccccc3-c3ccccc3-c3ccccc3)ccc2-c2cc3ccccc3cc21]

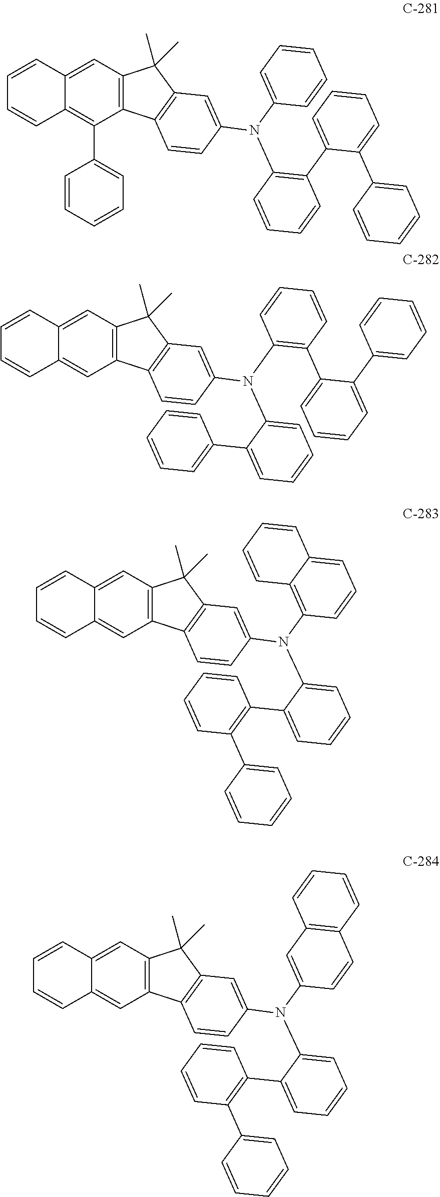

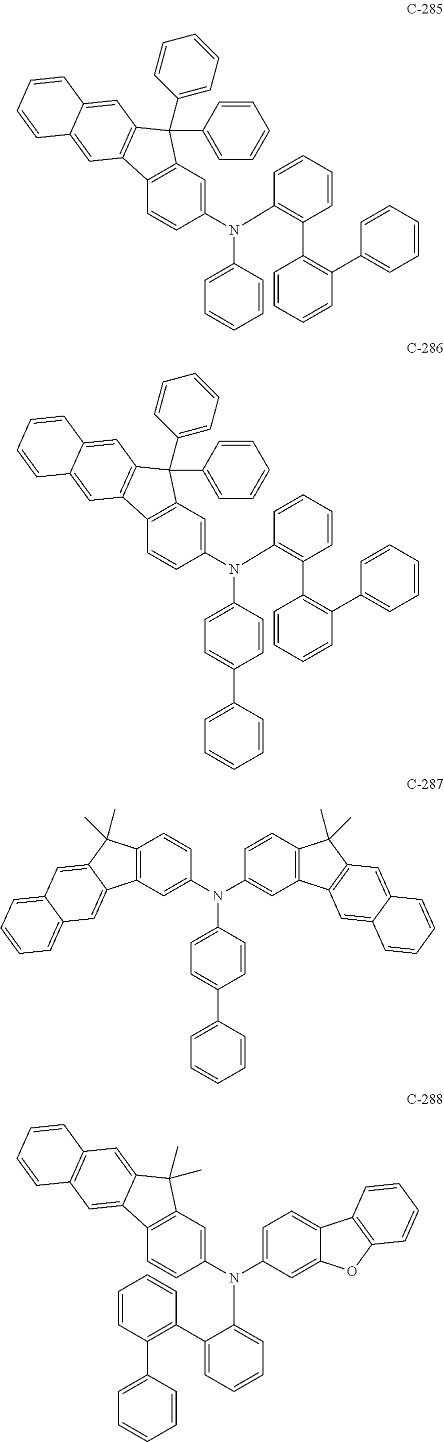 [SMILES: c1ccc(-c2ccccc2-c2ccccc2N(c2ccccc2)c2ccc3c(c2)C(c2ccccc2)(c2ccccc2)c2cc4ccccc4cc2-3)cc1 ; c1ccc(-c2ccc(N(c3ccc4c(c3)C(c3ccccc3)(c3ccccc3)c3cc5ccccc5cc3-4)c3ccccc3-c3ccccc3-c3ccccc3)cc2)cc1 ; CC1(C)c2ccc(N(c3ccc(-c4ccccc4)cc3)c3ccc4c(c3)-c3cc5ccccc5cc3C4(C)C)cc2-c2cc3ccccc3cc21 ; CC1(C)c2cc(N(c3ccc4c(c3)oc3ccccc34)c3ccccc3-c3ccccc3-c3ccccc3)ccc2-c2cc3ccccc3cc21]

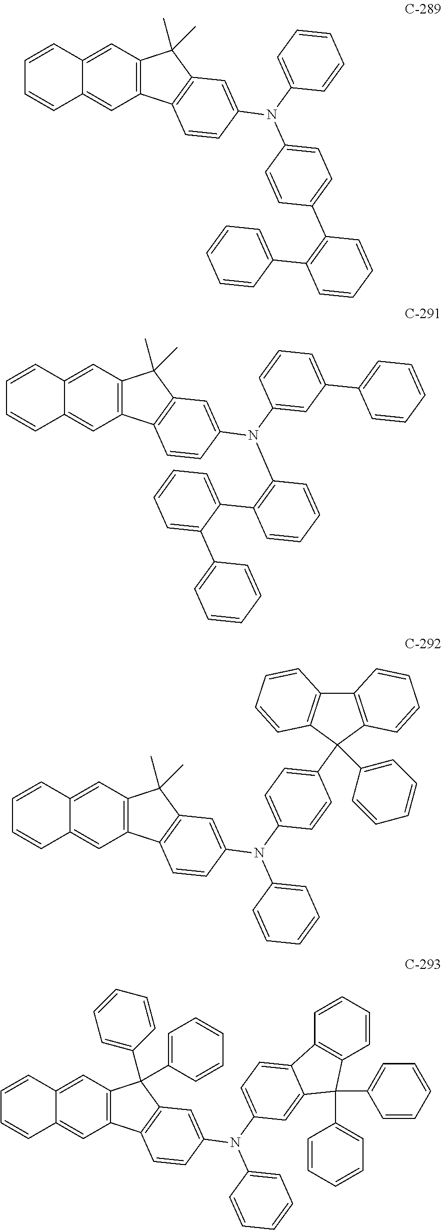 [SMILES: CC1(C)c2cc(N(c3ccccc3)c3ccc(-c4ccccc4-c4ccccc4)cc3)ccc2-c2cc3ccccc3cc21 ; CC1(C)c2cc(N(c3cccc(-c4ccccc4)c3)c3ccccc3-c3ccccc3-c3ccccc3)ccc2-c2cc3ccccc3cc21 ; CC1(C)c2cc(N(c3ccccc3)c3ccc(C4(c5ccccc5)c5ccccc5-c5ccccc54)cc3)ccc2-c2cc3ccccc3cc21 ; c1ccc(N(c2ccc3c(c2)C(c2ccccc2)(c2ccccc2)c2ccccc2-3)c2ccc3c(c2)C(c2ccccc2)(c2ccccc2)c2cc4ccccc4cc2-3)cc1]

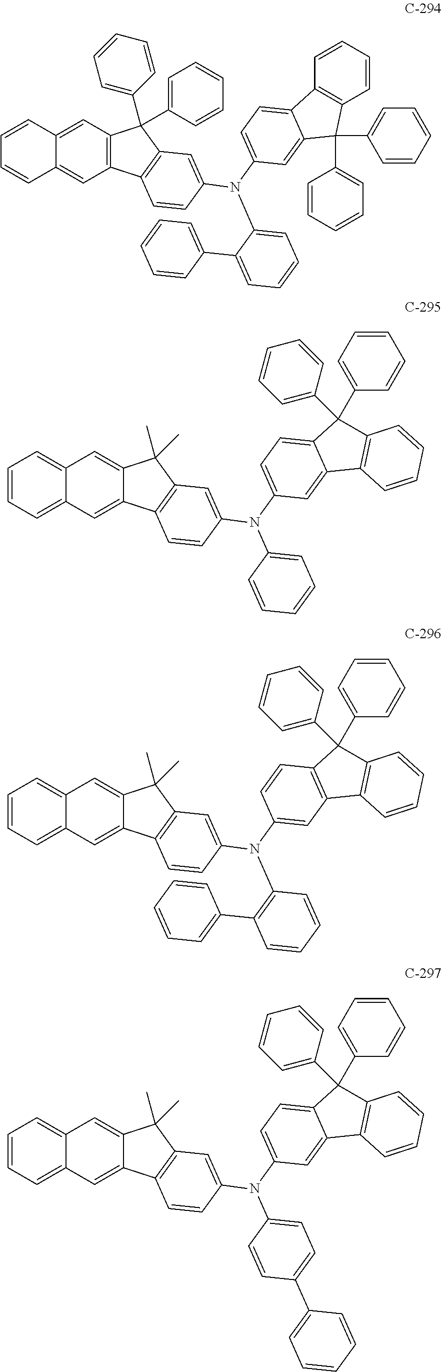 [SMILES: c1ccc(-c2ccccc2N(c2ccc3c(c2)C(c2ccccc2)(c2ccccc2)c2ccccc2-3)c2ccc3c(c2)C(c2ccccc2)(c2ccccc2)c2cc4ccccc4cc2-3)cc1 ; CC1(C)c2cc(N(c3ccccc3)c3ccc4c(c3)-c3ccccc3C4(c3ccccc3)c3ccccc3)ccc2-c2cc3ccccc3cc21 ; CC1(C)c2cc(N(c3ccc4c(c3)-c3ccccc3C4(c3ccccc3)c3ccccc3)c3ccccc3-c3ccccc3)ccc2-c2cc3ccccc3cc21 ; CC1(C)c2cc(N(c3ccc(-c4ccccc4)cc3)c3ccc4c(c3)-c3ccccc3C4(c3ccccc3)c3ccccc3)ccc2-c2cc3ccccc3cc21]

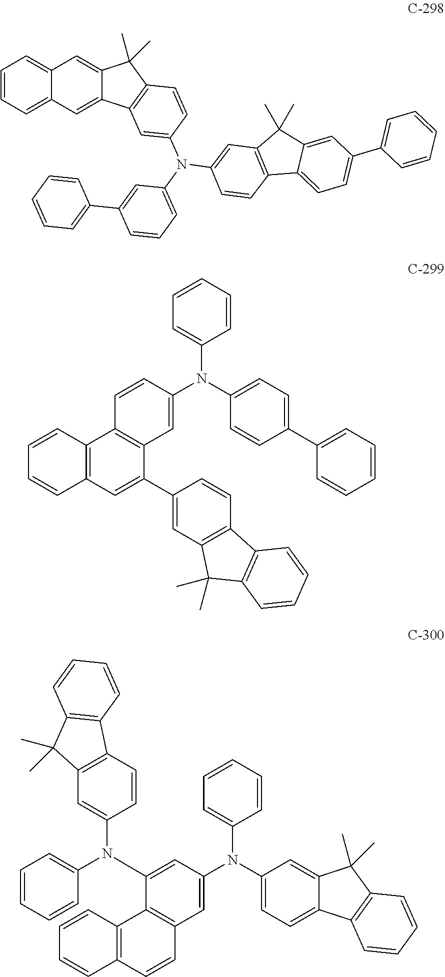 [SMILES: CC1(C)c2cc(-c3ccccc3)ccc2-c2ccc(N(c3cccc(-c4ccccc4)c3)c3ccc4c(c3)-c3cc5ccccc5cc3C4(C)C)cc21 ; CC1(C)c2ccccc2-c2ccc(-c3cc4ccccc4c4ccc(N(c5ccccc5)c5ccc(-c6ccccc6)cc5)cc34)cc21 ; CC1(C)c2ccccc2-c2ccc(N(c3ccccc3)c3cc(N(c4ccccc4)c4ccc5c(c4)C(C)(C)c4ccccc4-5)c4c(ccc5ccccc54)c3)cc21]

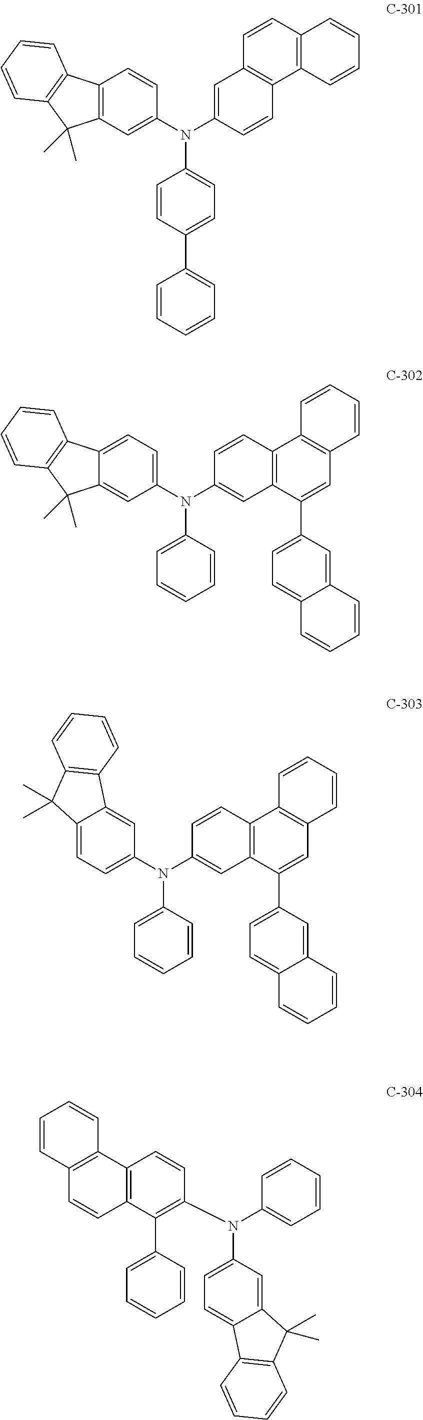 [SMILES: CC1(C)c2ccccc2-c2ccc(N(c3ccc(-c4ccccc4)cc3)c3ccc4c(ccc5ccccc54)c3)cc21 ; CC1(C)c2ccccc2-c2ccc(N(c3ccccc3)c3ccc4c(c3)c(-c3ccc5ccccc5c3)cc3ccccc34)cc21 ; CC1(C)c2ccccc2-c2cc(N(c3ccccc3)c3ccc4c(c3)c(-c3ccc5ccccc5c3)cc3ccccc34)ccc21 ; CC1(C)c2ccccc2-c2ccc(N(c3ccccc3)c3ccc4c(ccc5ccccc54)c3-c3ccccc3)cc21]

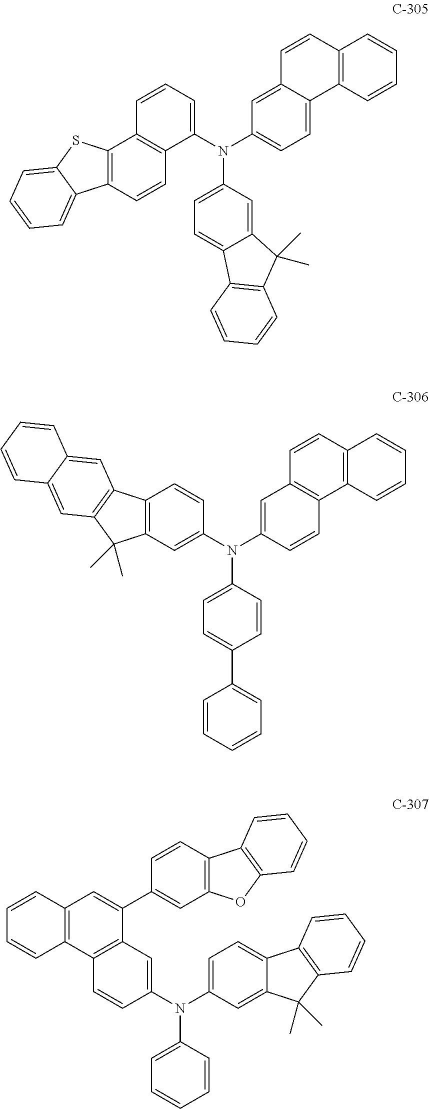 [SMILES: CC1(C)c2ccccc2-c2ccc(N(c3ccc4c(ccc5ccccc54)c3)c3cccc4c3ccc3c5ccccc5sc43)cc21 ; CC1(C)c2cc(N(c3ccc(-c4ccccc4)cc3)c3ccc4c(ccc5ccccc54)c3)ccc2-c2cc3ccccc3cc21 ; CC1(C)c2ccccc2-c2ccc(N(c3ccccc3)c3ccc4c(c3)c(-c3ccc5c(c3)oc3ccccc35)cc3ccccc34)cc21]

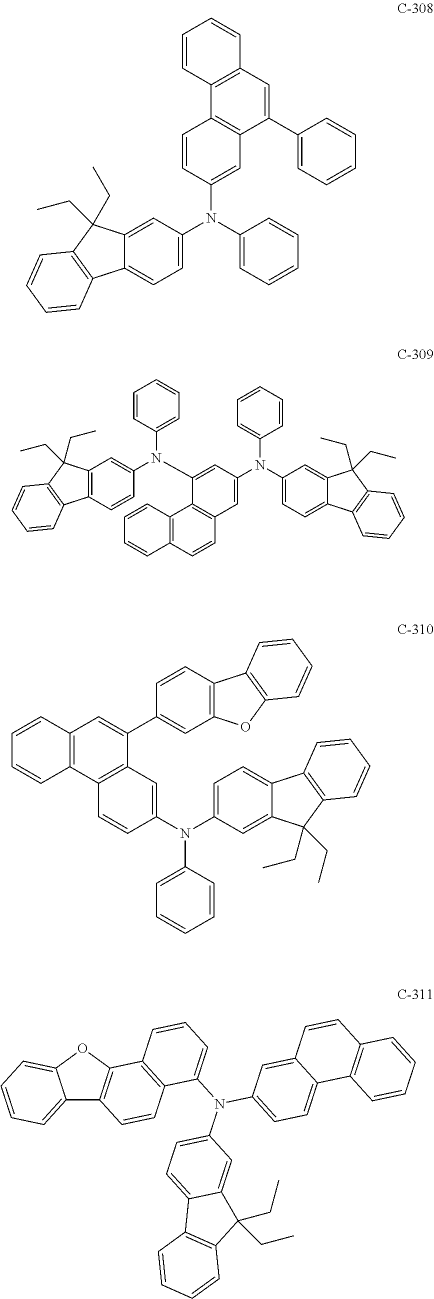 [SMILES: CCC1(CC)c2ccccc2-c2ccc(N(c3ccccc3)c3ccc4c(c3)c(-c3ccccc3)cc3ccccc34)cc21 ; CCC1(CC)c2ccccc2-c2ccc(N(c3ccccc3)c3cc(N(c4ccccc4)c4ccc5c(c4)C(CC)(CC)c4ccccc4-5)c4c(ccc5ccccc54)c3)cc21 ; CCC1(CC)c2ccccc2-c2ccc(N(c3ccccc3)c3ccc4c(c3)c(-c3ccc5c(c3)oc3ccccc35)cc3ccccc34)cc21 ; CCC1(CC)c2ccccc2-c2ccc(N(c3ccc4c(ccc5ccccc54)c3)c3cccc4c3ccc3c5ccccc5oc43)cc21]

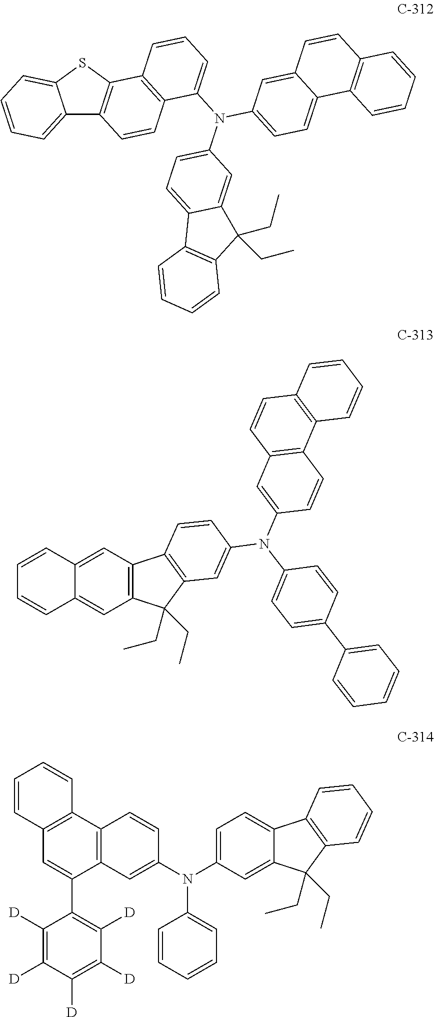 [SMILES: CCC1(CC)c2ccccc2-c2ccc(N(c3ccc4c(ccc5ccccc54)c3)c3cccc4c3ccc3c5ccccc5sc43)cc21 ; CCC1(CC)c2cc(N(c3ccc(-c4ccccc4)cc3)c3ccc4c(ccc5ccccc54)c3)ccc2-c2cc3ccccc3cc21 ; [2H]c1c([2H])c([2H])c(-c2cc3ccccc3c3ccc(N(c4ccccc4)c4ccc5c(c4)C(CC)(CC)c4ccccc4-5)cc23)c([2H])c1[2H]]

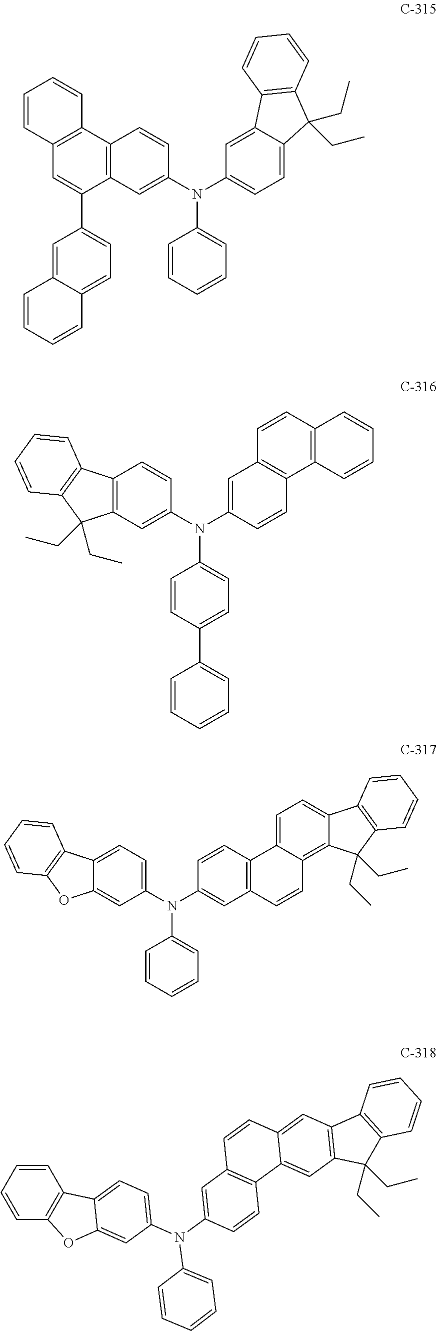 [SMILES: CCC1(CC)c2ccccc2-c2cc(N(c3ccccc3)c3ccc4c(c3)c(-c3ccc5ccccc5c3)cc3ccccc34)ccc21 ; CCC1(CC)c2ccccc2-c2ccc(N(c3ccc(-c4ccccc4)cc3)c3ccc4c(ccc5ccccc54)c3)cc21 ; CCC1(CC)c2ccccc2-c2ccc3c(ccc4cc(N(c5ccccc5)c5ccc6c(c5)oc5ccccc56)ccc43)c21 ; CCC1(CC)c2ccccc2-c2cc3ccc4cc(N(c5ccccc5)c5ccc6c(c5)oc5ccccc56)ccc4c3cc21]

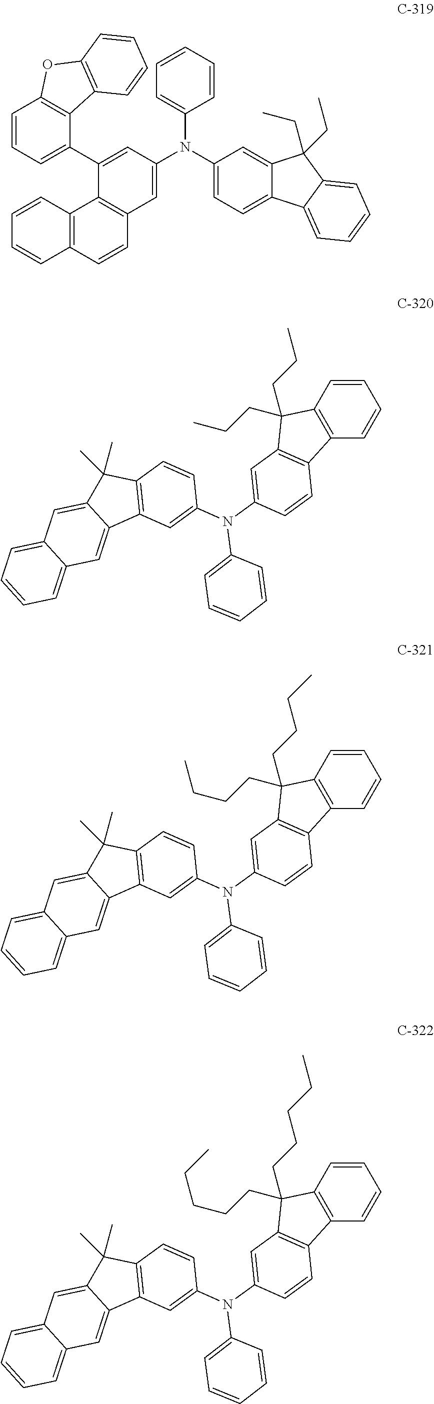 [SMILES: CCC1(CC)c2ccccc2-c2ccc(N(c3ccccc3)c3cc(-c4cccc5oc6ccccc6c45)c4c(ccc5ccccc54)c3)cc21 ; CCCC1(CCC)c2ccccc2-c2ccc(N(c3ccccc3)c3ccc4c(c3)-c3cc5ccccc5cc3C4(C)C)cc21 ; CCCCC1(CCCC)c2ccccc2-c2ccc(N(c3ccccc3)c3ccc4c(c3)-c3cc5ccccc5cc3C4(C)C)cc21 ; CCCCCC1(CCCCC)c2ccccc2-c2ccc(N(c3ccccc3)c3ccc4c(c3)-c3cc5ccccc5cc3C4(C)C)cc21]

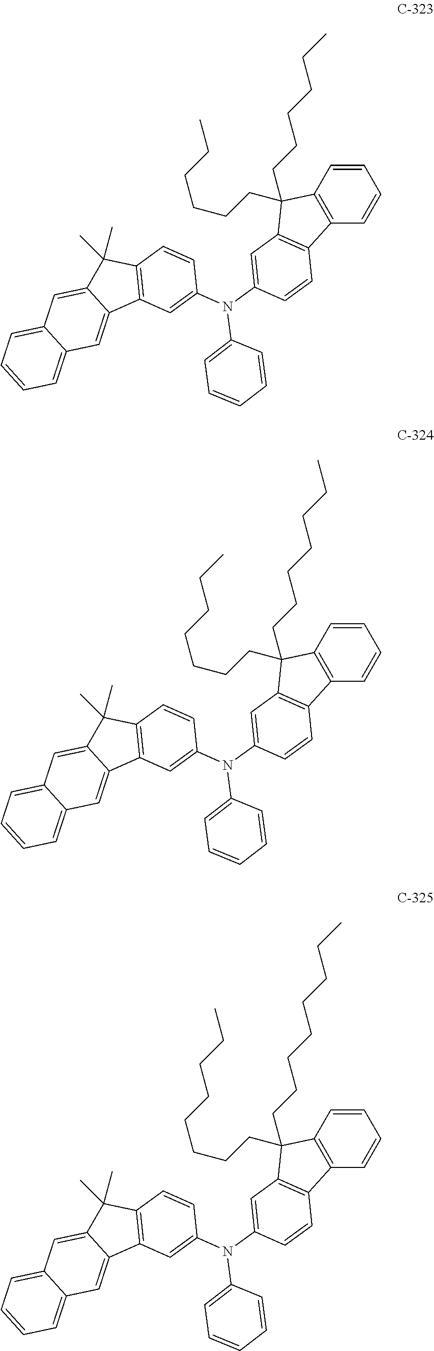 [SMILES: CCCCCCC1(CCCCCC)c2ccccc2-c2ccc(N(c3ccccc3)c3ccc4c(c3)-c3cc5ccccc5cc3C4(C)C)cc21 ; CCCCCCCC1(CCCCCCC)c2ccccc2-c2ccc(N(c3ccccc3)c3ccc4c(c3)-c3cc5ccccc5cc3C4(C)C)cc21 ; CCCCCCCCC1(CCCCCCCC)c2ccccc2-c2ccc(N(c3ccccc3)c3ccc4c(c3)-c3cc5ccccc5cc3C4(C)C)cc21]

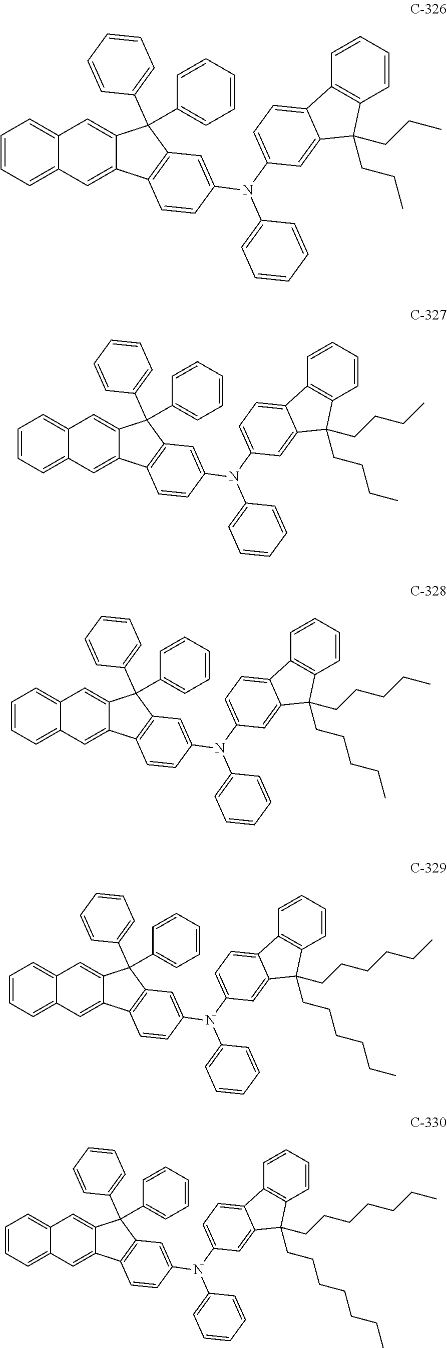 [SMILES: CCCC1(CCC)c2ccccc2-c2ccc(N(c3ccccc3)c3ccc4c(c3)C(c3ccccc3)(c3ccccc3)c3cc5ccccc5cc3-4)cc21 ; CCCCC1(CCCC)c2ccccc2-c2ccc(N(c3ccccc3)c3ccc4c(c3)C(c3ccccc3)(c3ccccc3)c3cc5ccccc5cc3-4)cc21 ; CCCCCC1(CCCCC)c2ccccc2-c2ccc(N(c3ccccc3)c3ccc4c(c3)C(c3ccccc3)(c3ccccc3)c3cc5ccccc5cc3-4)cc21 ; CCCCCCC1(CCCCCC)c2ccccc2-c2ccc(N(c3ccccc3)c3ccc4c(c3)C(c3ccccc3)(c3ccccc3)c3cc5ccccc5cc3-4)cc21 ; CCCCCCCC1(CCCCCCC)c2ccccc2-c2ccc(N(c3ccccc3)c3ccc4c(c3)C(c3ccccc3)(c3ccccc3)c3cc5ccccc5cc3-4)cc21]

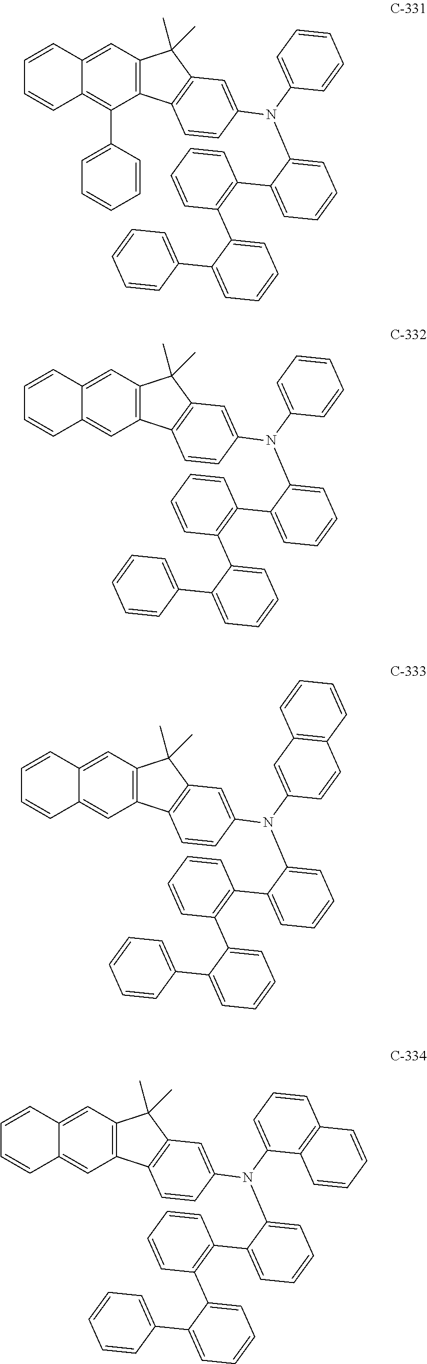 [SMILES: CC1(C)c2cc(N(c3ccccc3)c3ccccc3-c3ccccc3-c3ccccc3-c3ccccc3)ccc2-c2c1cc1ccccc1c2-c1ccccc1 ; CC1(C)c2cc(N(c3ccccc3)c3ccccc3-c3ccccc3-c3ccccc3-c3ccccc3)ccc2-c2cc3ccccc3cc21 ; CC1(C)c2cc(N(c3ccc4ccccc4c3)c3ccccc3-c3ccccc3-c3ccccc3-c3ccccc3)ccc2-c2cc3ccccc3cc21 ; CC1(C)c2cc(N(c3ccccc3-c3ccccc3-c3ccccc3-c3ccccc3)c3cccc4ccccc34)ccc2-c2cc3ccccc3cc21]

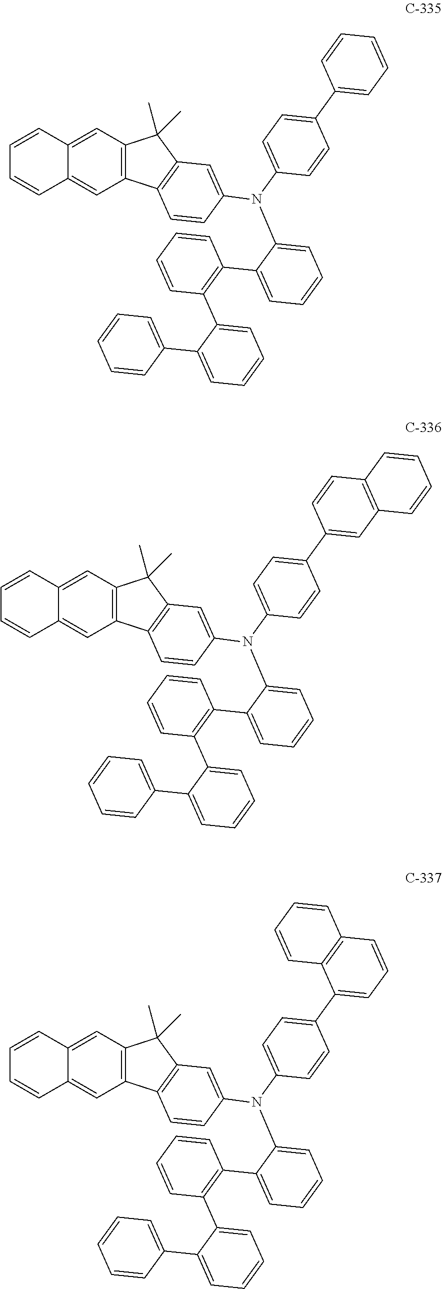 [SMILES: CC1(C)c2cc(N(c3ccc(-c4ccccc4)cc3)c3ccccc3-c3ccccc3-c3ccccc3-c3ccccc3)ccc2-c2cc3ccccc3cc21 ; CC1(C)c2cc(N(c3ccc(-c4ccc5ccccc5c4)cc3)c3ccccc3-c3ccccc3-c3ccccc3-c3ccccc3)ccc2-c2cc3ccccc3cc21 ; CC1(C)c2cc(N(c3ccc(-c4cccc5ccccc45)cc3)c3ccccc3-c3ccccc3-c3ccccc3-c3ccccc3)ccc2-c2cc3ccccc3cc21]

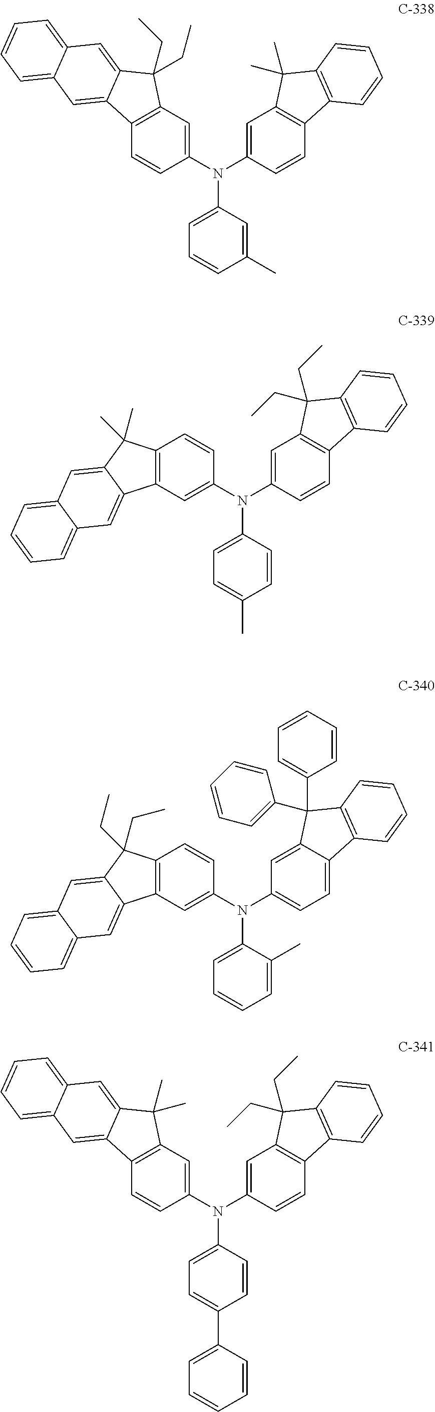 [SMILES: CCC1(CC)c2cc(N(c3cccc(C)c3)c3ccc4c(c3)C(C)(C)c3ccccc3-4)ccc2-c2cc3ccccc3cc21 ; CCC1(CC)c2ccccc2-c2ccc(N(c3ccc(C)cc3)c3ccc4c(c3)-c3cc5ccccc5cc3C4(C)C)cc21 ; CCC1(CC)c2ccc(N(c3ccc4c(c3)C(c3ccccc3)(c3ccccc3)c3ccccc3-4)c3ccccc3C)cc2-c2cc3ccccc3cc21 ; CCC1(CC)c2ccccc2-c2ccc(N(c3ccc(-c4ccccc4)cc3)c3ccc4c(c3)C(C)(C)c3cc5ccccc5cc3-4)cc21]

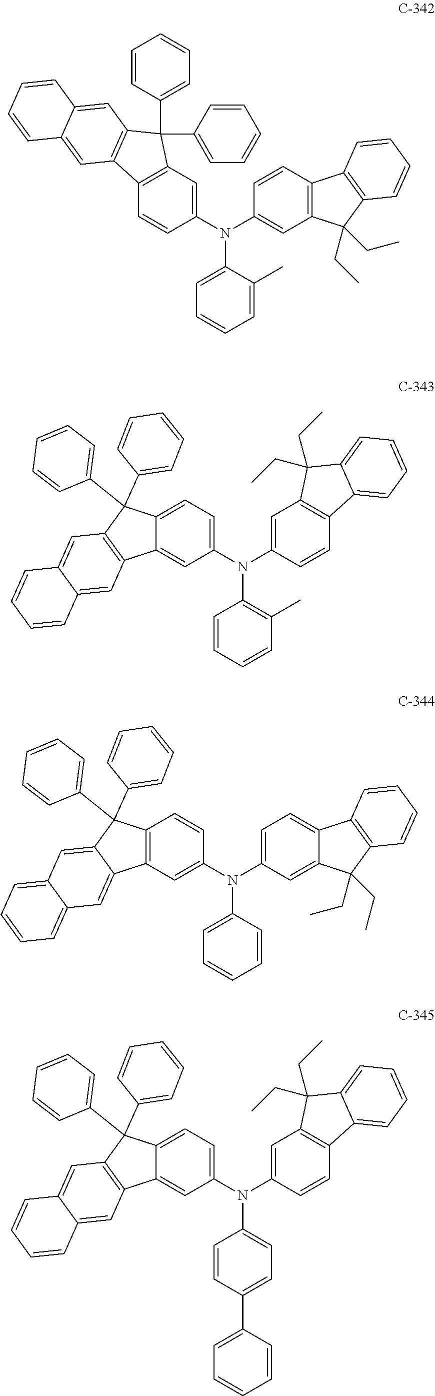 [SMILES: CCC1(CC)c2ccccc2-c2ccc(N(c3ccc4c(c3)C(c3ccccc3)(c3ccccc3)c3cc5ccccc5cc3-4)c3ccccc3C)cc21 ; CCC1(CC)c2ccccc2-c2ccc(N(c3ccc4c(c3)-c3cc5ccccc5cc3C4(c3ccccc3)c3ccccc3)c3ccccc3C)cc21 ; CCC1(CC)c2ccccc2-c2ccc(N(c3ccccc3)c3ccc4c(c3)-c3cc5ccccc5cc3C4(c3ccccc3)c3ccccc3)cc21 ; CCC1(CC)c2ccccc2-c2ccc(N(c3ccc(-c4ccccc4)cc3)c3ccc4c(c3)-c3cc5ccccc5cc3C4(c3ccccc3)c3ccccc3)cc21]

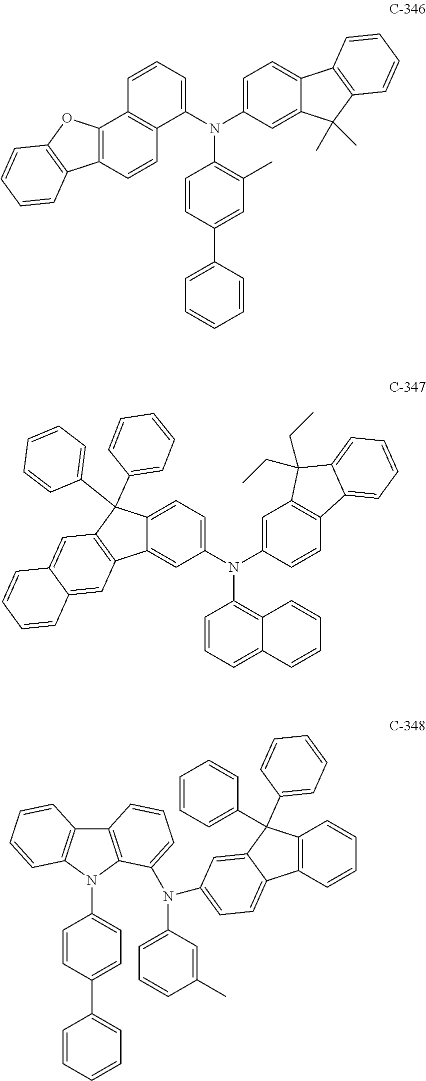 [SMILES: Cc1cc(-c2ccccc2)ccc1N(c1ccc2c(c1)C(C)(C)c1ccccc1-2)c1cccc2c1ccc1c3ccccc3oc21 ; CCC1(CC)c2ccccc2-c2ccc(N(c3ccc4c(c3)-c3cc5ccccc5cc3C4(c3ccccc3)c3ccccc3)c3cccc4ccccc34)cc21 ; Cc1cccc(N(c2ccc3c(c2)C(c2ccccc2)(c2ccccc2)c2ccccc2-3)c2cccc3c4ccccc4n(-c4ccc(-c5ccccc5)cc4)c23)c1]

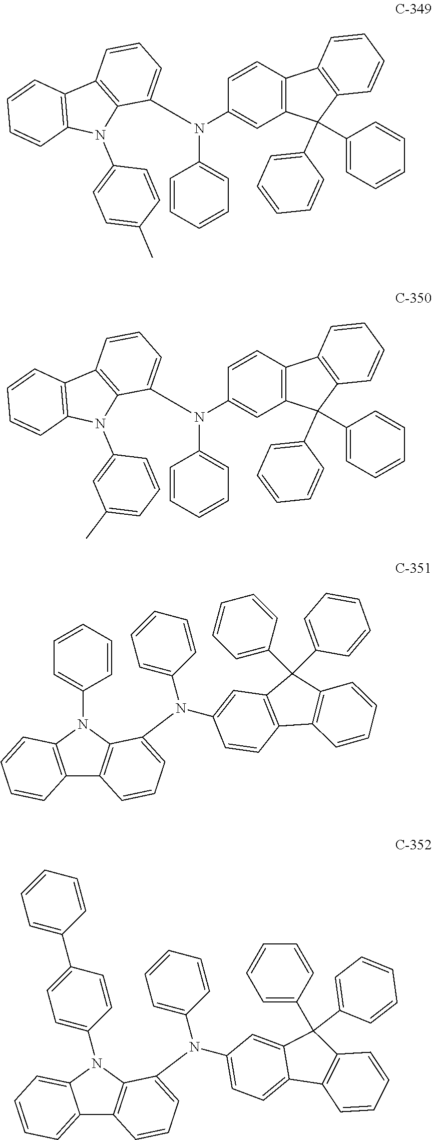 [SMILES: Cc1ccc(-n2c3ccccc3c3cccc(N(c4ccccc4)c4ccc5c(c4)C(c4ccccc4)(c4ccccc4)c4ccccc4-5)c32)cc1 ; Cc1cccc(-n2c3ccccc3c3cccc(N(c4ccccc4)c4ccc5c(c4)C(c4ccccc4)(c4ccccc4)c4ccccc4-5)c32)c1 ; c1ccc(N(c2ccc3c(c2)C(c2ccccc2)(c2ccccc2)c2ccccc2-3)c2cccc3c4ccccc4n(-c4ccccc4)c23)cc1 ; c1ccc(-c2ccc(-n3c4ccccc4c4cccc(N(c5ccccc5)c5ccc6c(c5)C(c5ccccc5)(c5ccccc5)c5ccccc5-6)c43)cc2)cc1]

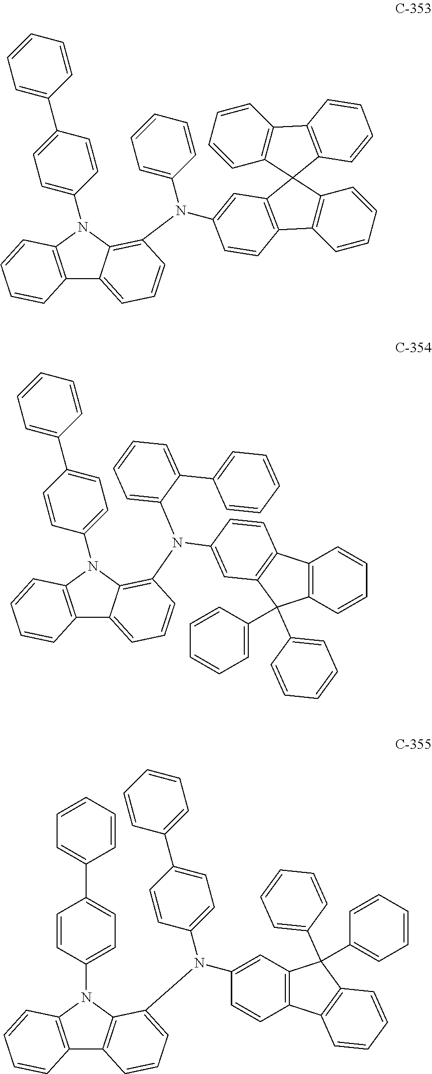 [SMILES: c1ccc(-c2ccc(-n3c4ccccc4c4cccc(N(c5ccccc5)c5ccc6c(c5)C5(c7ccccc7-c7ccccc75)c5ccccc5-6)c43)cc2)cc1 ; c1ccc(-c2ccc(-n3c4ccccc4c4cccc(N(c5ccc6c(c5)C(c5ccccc5)(c5ccccc5)c5ccccc5-6)c5ccccc5-c5ccccc5)c43)cc2)cc1 ; c1ccc(-c2ccc(N(c3ccc4c(c3)C(c3ccccc3)(c3ccccc3)c3ccccc3-4)c3cccc4c5ccccc5n(-c5ccc(-c6ccccc6)cc5)c34)cc2)cc1]

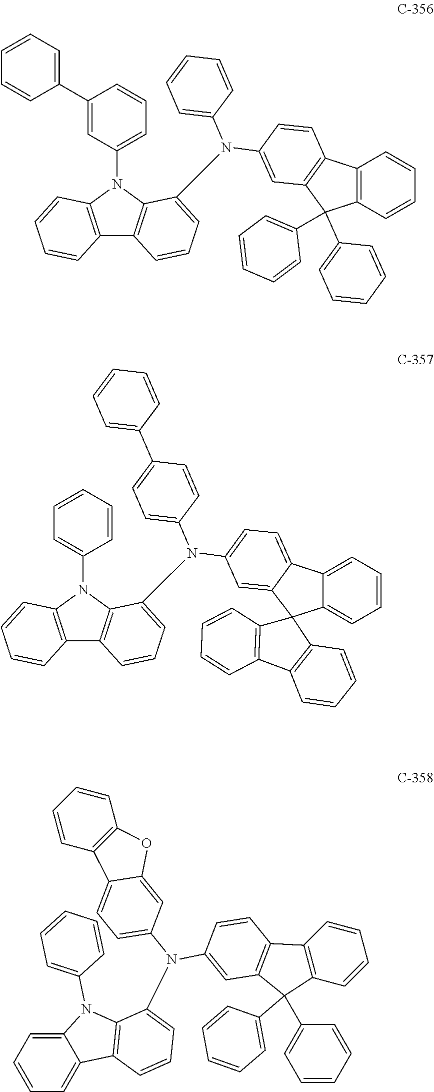 [SMILES: c1ccc(-c2cccc(-n3c4ccccc4c4cccc(N(c5ccccc5)c5ccc6c(c5)C(c5ccccc5)(c5ccccc5)c5ccccc5-6)c43)c2)cc1 ; c1ccc(-c2ccc(N(c3ccc4c(c3)C3(c5ccccc5-c5ccccc53)c3ccccc3-4)c3cccc4c5ccccc5n(-c5ccccc5)c34)cc2)cc1 ; c1ccc(-n2c3ccccc3c3cccc(N(c4ccc5c(c4)C(c4ccccc4)(c4ccccc4)c4ccccc4-5)c4ccc5c(c4)oc4ccccc45)c32)cc1]

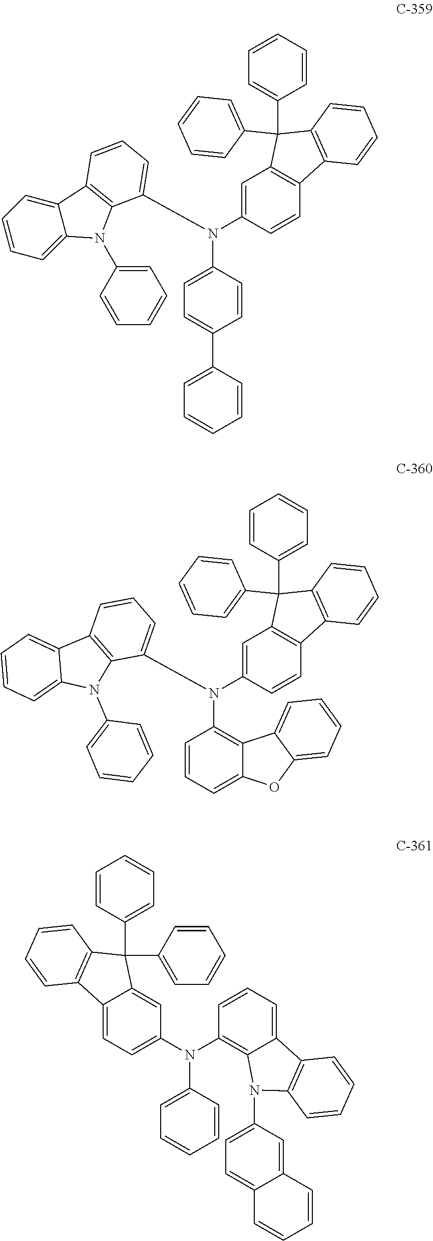 [SMILES: c1ccc(-c2ccc(N(c3ccc4c(c3)C(c3ccccc3)(c3ccccc3)c3ccccc3-4)c3cccc4c5ccccc5n(-c5ccccc5)c34)cc2)cc1 ; c1ccc(-n2c3ccccc3c3cccc(N(c4ccc5c(c4)C(c4ccccc4)(c4ccccc4)c4ccccc4-5)c4cccc5oc6ccccc6c45)c32)cc1 ; c1ccc(N(c2ccc3c(c2)C(c2ccccc2)(c2ccccc2)c2ccccc2-3)c2cccc3c4ccccc4n(-c4ccc5ccccc5c4)c23)cc1]

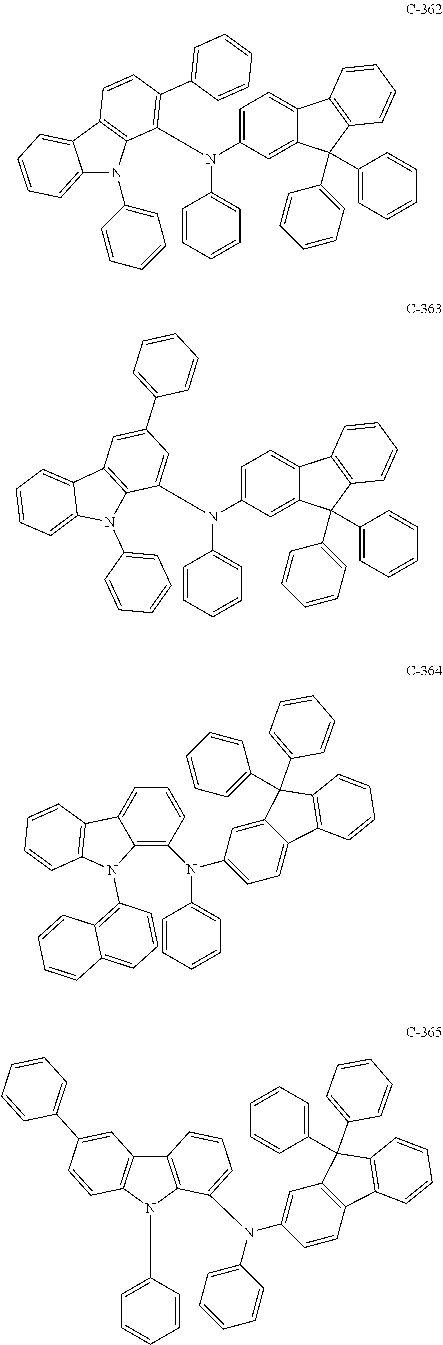 [SMILES: c1ccc(-c2ccc3c4ccccc4n(-c4ccccc4)c3c2N(c2ccccc2)c2ccc3c(c2)C(c2ccccc2)(c2ccccc2)c2ccccc2-3)cc1 ; c1ccc(-c2cc(N(c3ccccc3)c3ccc4c(c3)C(c3ccccc3)(c3ccccc3)c3ccccc3-4)c3c(c2)c2ccccc2n3-c2ccccc2)cc1 ; c1ccc(N(c2ccc3c(c2)C(c2ccccc2)(c2ccccc2)c2ccccc2-3)c2cccc3c4ccccc4n(-c4cccc5ccccc45)c23)cc1 ; c1ccc(-c2ccc3c(c2)c2cccc(N(c4ccccc4)c4ccc5c(c4)C(c4ccccc4)(c4ccccc4)c4ccccc4-5)c2n3-c2ccccc2)cc1]

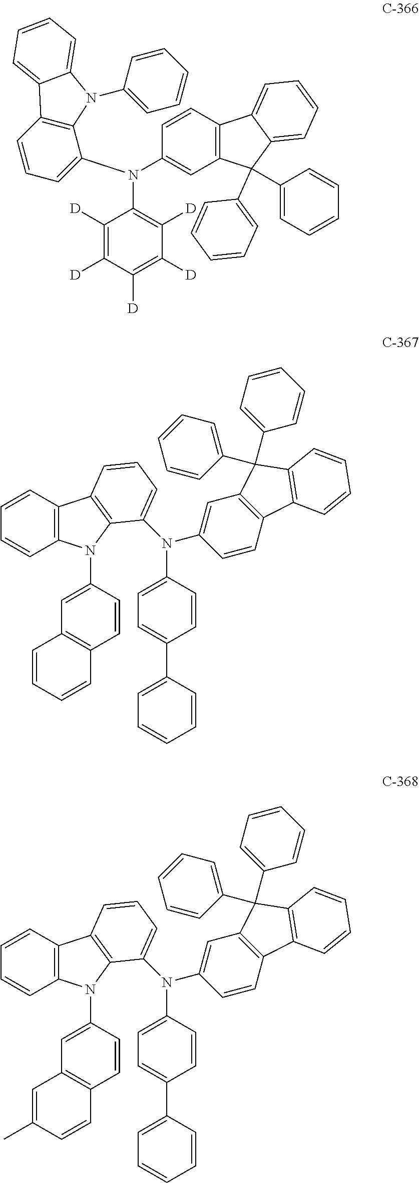 [SMILES: [2H]c1c([2H])c([2H])c(N(c2ccc3c(c2)C(c2ccccc2)(c2ccccc2)c2ccccc2-3)c2cccc3c4ccccc4n(-c4ccccc4)c23)c([2H])c1[2H] ; c1ccc(-c2ccc(N(c3ccc4c(c3)C(c3ccccc3)(c3ccccc3)c3ccccc3-4)c3cccc4c5ccccc5n(-c5ccc6ccccc6c5)c34)cc2)cc1 ; Cc1ccc2ccc(-n3c4ccccc4c4cccc(N(c5ccc(-c6ccccc6)cc5)c5ccc6c(c5)C(c5ccccc5)(c5ccccc5)c5ccccc5-6)c43)cc2c1]

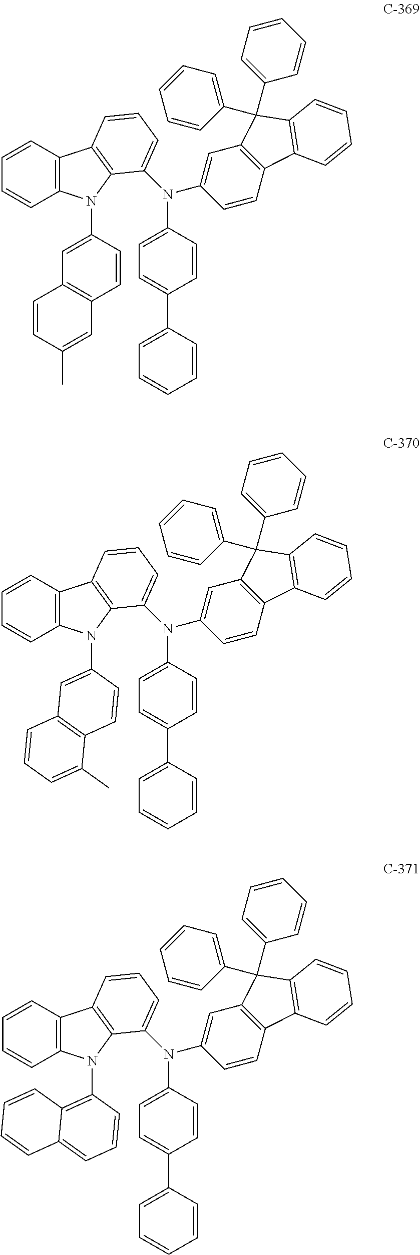 [SMILES: Cc1ccc2cc(-n3c4ccccc4c4cccc(N(c5ccc(-c6ccccc6)cc5)c5ccc6c(c5)C(c5ccccc5)(c5ccccc5)c5ccccc5-6)c43)ccc2c1 ; Cc1cccc2cc(-n3c4ccccc4c4cccc(N(c5ccc(-c6ccccc6)cc5)c5ccc6c(c5)C(c5ccccc5)(c5ccccc5)c5ccccc5-6)c43)ccc12 ; c1ccc(-c2ccc(N(c3ccc4c(c3)C(c3ccccc3)(c3ccccc3)c3ccccc3-4)c3cccc4c5ccccc5n(-c5cccc6ccccc56)c34)cc2)cc1]

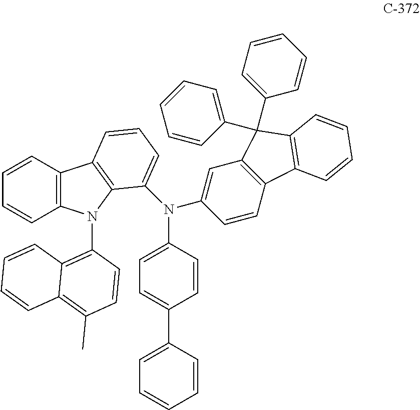 [SMILES: Cc1ccc(-n2c3ccccc3c3cccc(N(c4ccc(-c5ccccc5)cc4)c4ccc5c(c4)C(c4ccccc4)(c4ccccc4)c4ccccc4-5)c32)c2ccccc12]

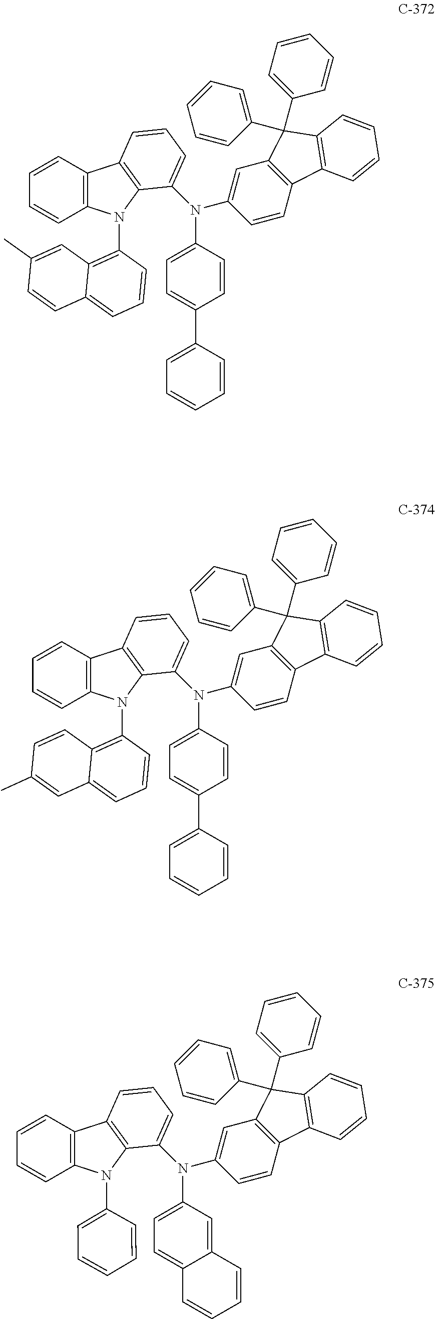 [SMILES: Cc1ccc2cccc(-n3c4ccccc4c4cccc(N(c5ccc(-c6ccccc6)cc5)c5ccc6c(c5)C(c5ccccc5)(c5ccccc5)c5ccccc5-6)c43)c2c1 ; Cc1ccc2c(-n3c4ccccc4c4cccc(N(c5ccc(-c6ccccc6)cc5)c5ccc6c(c5)C(c5ccccc5)(c5ccccc5)c5ccccc5-6)c43)cccc2c1 ; c1ccc(-n2c3ccccc3c3cccc(N(c4ccc5c(c4)C(c4ccccc4)(c4ccccc4)c4ccccc4-5)c4ccc5ccccc5c4)c32)cc1]

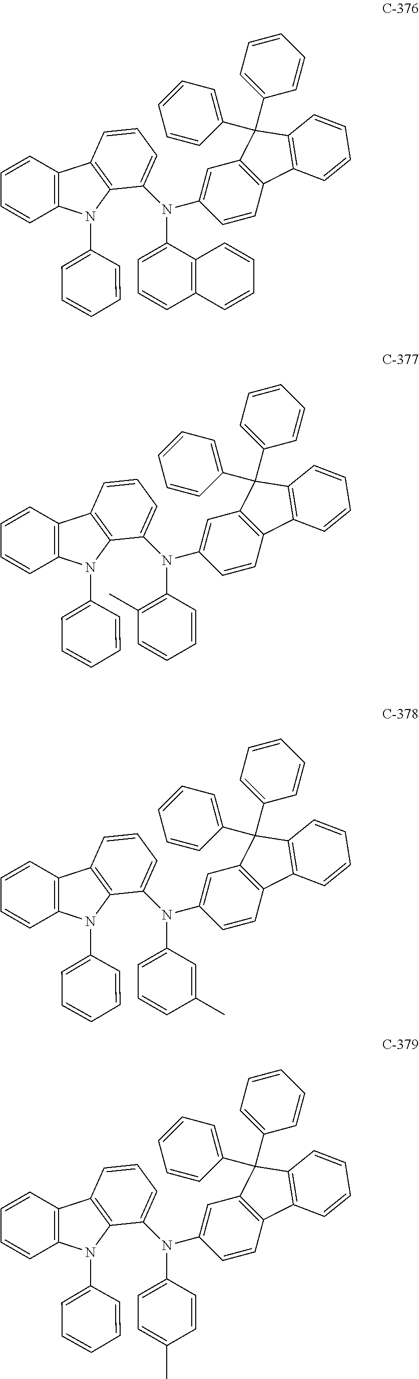 [SMILES: c1ccc(-n2c3ccccc3c3cccc(N(c4ccc5c(c4)C(c4ccccc4)(c4ccccc4)c4ccccc4-5)c4cccc5ccccc45)c32)cc1 ; Cc1ccccc1N(c1ccc2c(c1)C(c1ccccc1)(c1ccccc1)c1ccccc1-2)c1cccc2c3ccccc3n(-c3ccccc3)c12 ; Cc1cccc(N(c2ccc3c(c2)C(c2ccccc2)(c2ccccc2)c2ccccc2-3)c2cccc3c4ccccc4n(-c4ccccc4)c23)c1 ; Cc1ccc(N(c2ccc3c(c2)C(c2ccccc2)(c2ccccc2)c2ccccc2-3)c2cccc3c4ccccc4n(-c4ccccc4)c23)cc1]

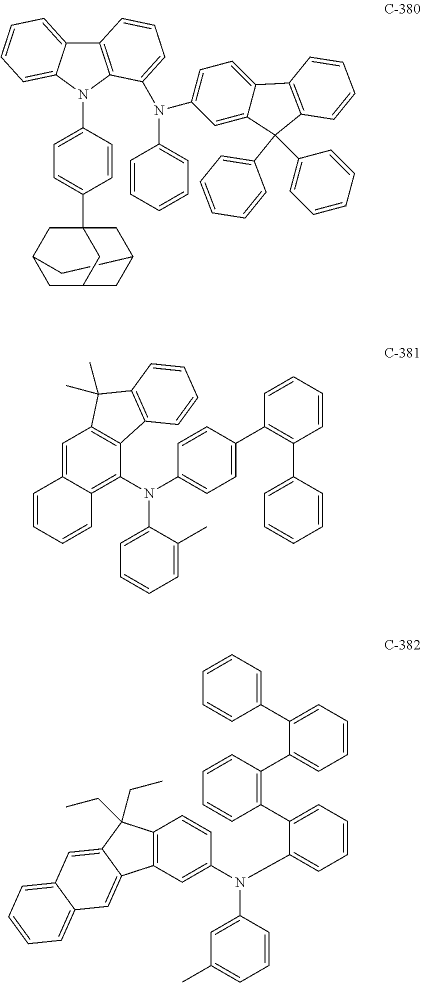 [SMILES: c1ccc(N(c2ccc3c(c2)C(c2ccccc2)(c2ccccc2)c2ccccc2-3)c2cccc3c4ccccc4n(-c4ccc(C56CC7CC(CC(C7)C5)C6)cc4)c23)cc1 ; Cc1ccccc1N(c1ccc(-c2ccccc2-c2ccccc2)cc1)c1c2c(cc3ccccc13)C(C)(C)c1ccccc1-2 ; CCC1(CC)c2ccc(N(c3cccc(C)c3)c3ccccc3-c3ccccc3-c3ccccc3-c3ccccc3)cc2-c2cc3ccccc3cc21]

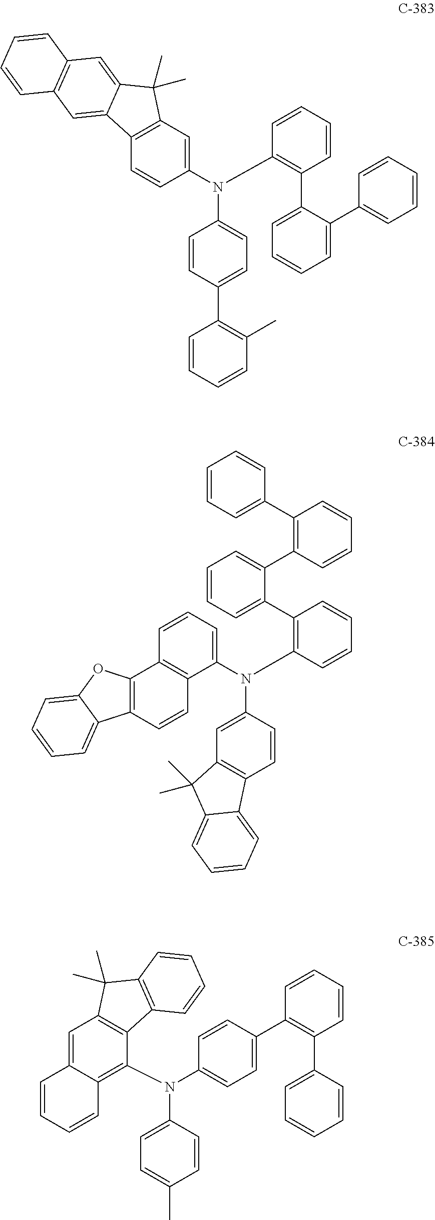 [SMILES: Cc1ccccc1-c1ccc(N(c2ccc3c(c2)C(C)(C)c2cc4ccccc4cc2-3)c2ccccc2-c2ccccc2-c2ccccc2)cc1 ; CC1(C)c2ccccc2-c2ccc(N(c3ccccc3-c3ccccc3-c3ccccc3-c3ccccc3)c3cccc4c3ccc3c5ccccc5oc43)cc21 ; Cc1ccc(N(c2ccc(-c3ccccc3-c3ccccc3)cc2)c2c3c(cc4ccccc24)C(C)(C)c2ccccc2-3)cc1]

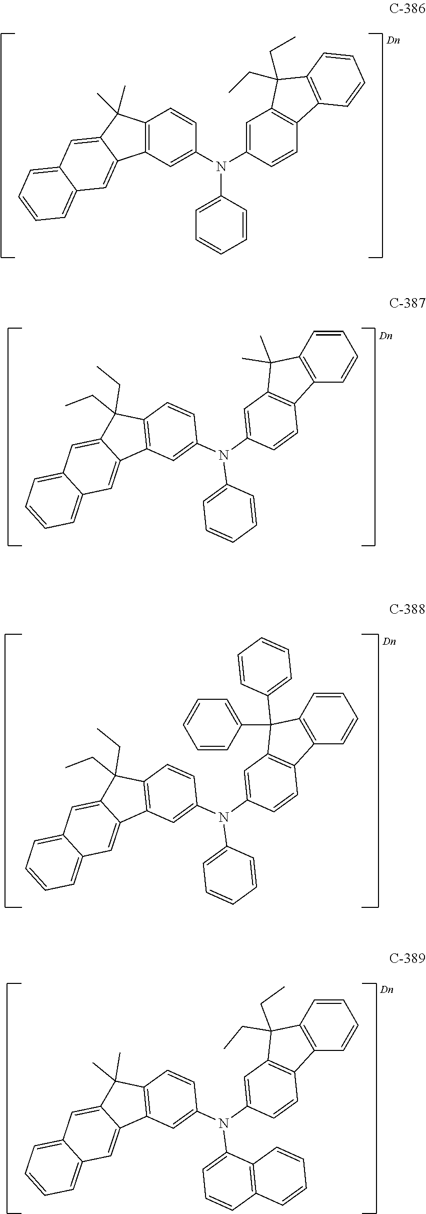

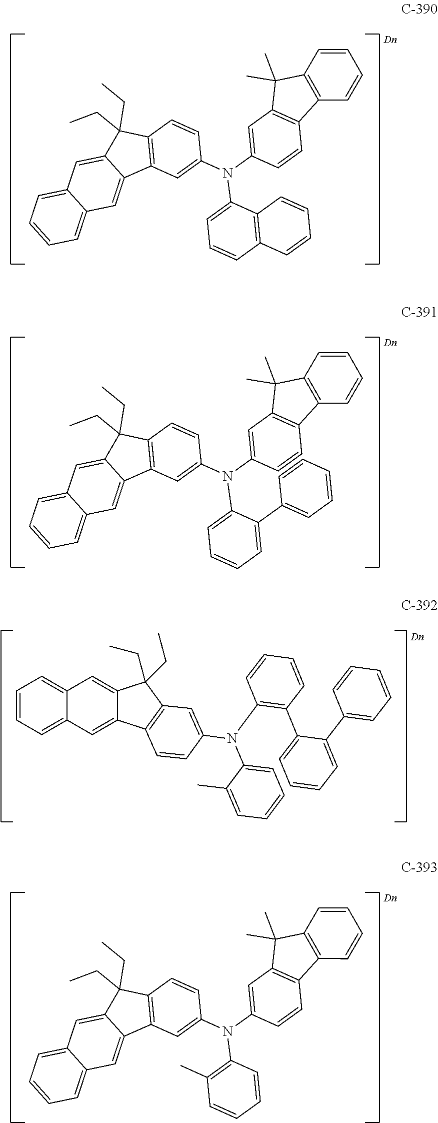

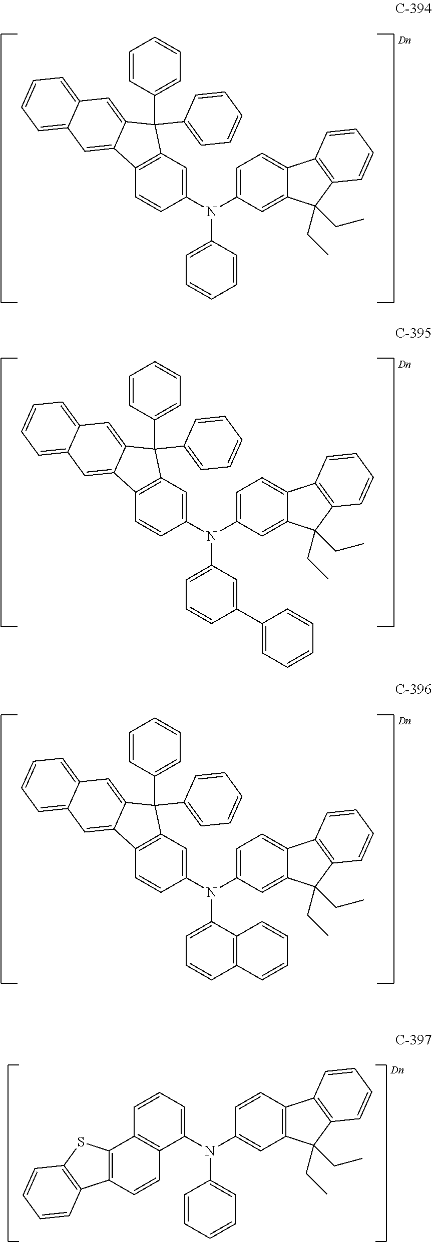

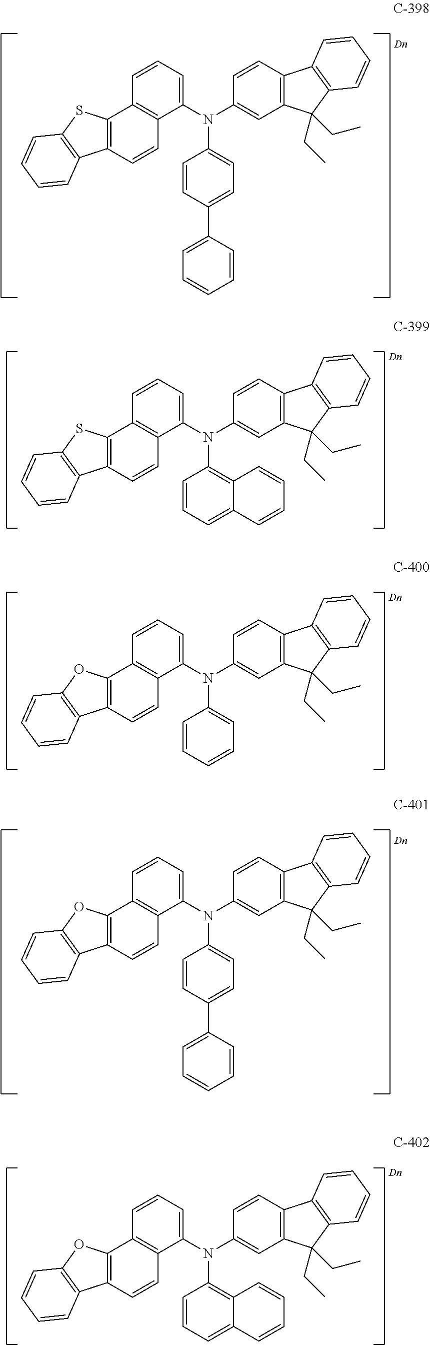

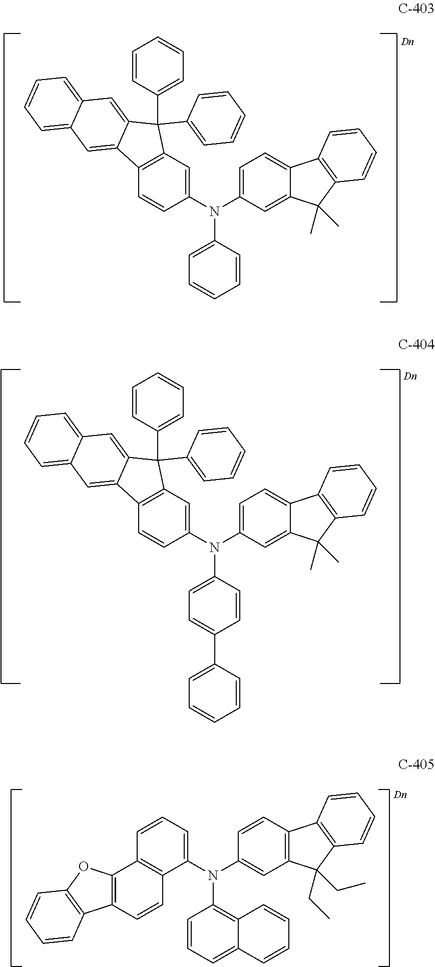

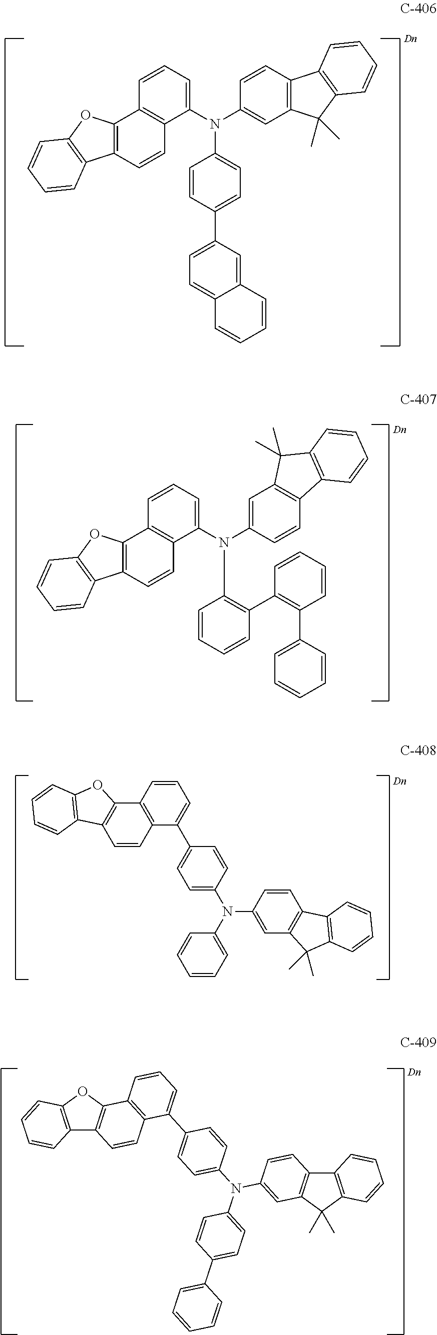

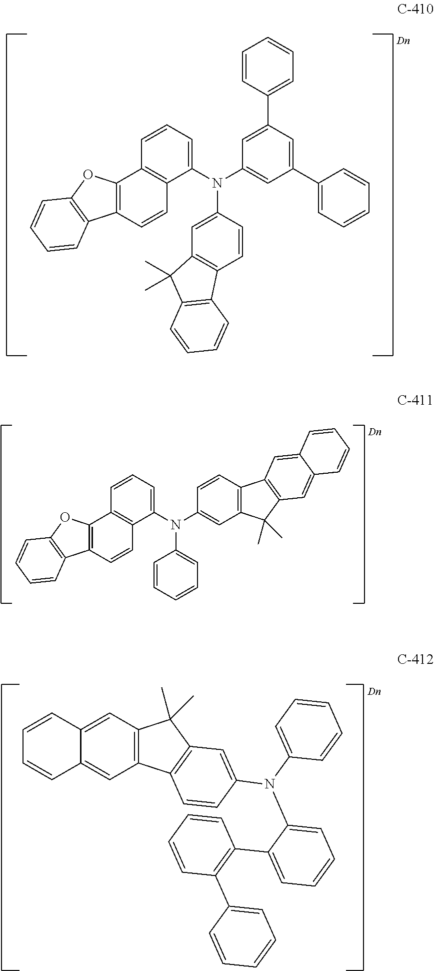

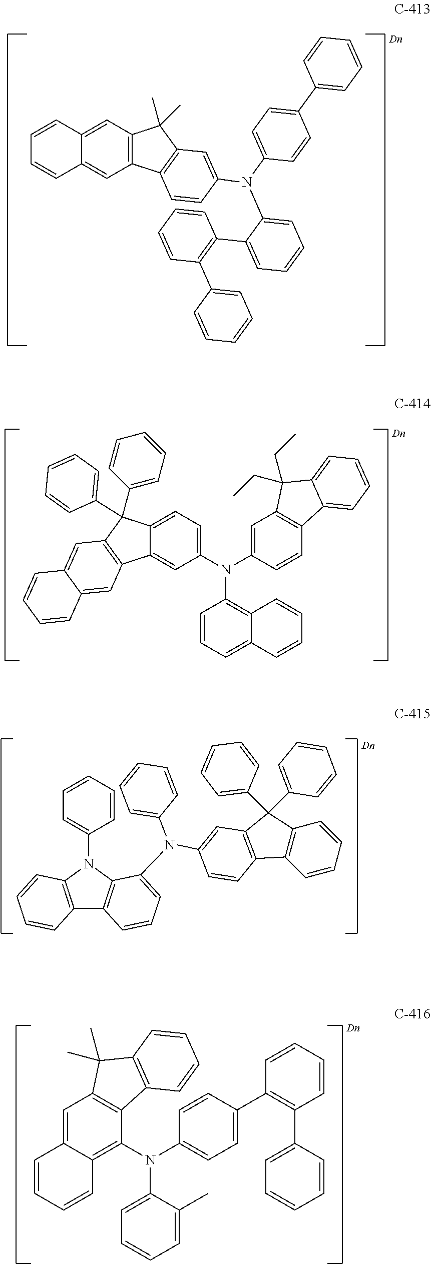

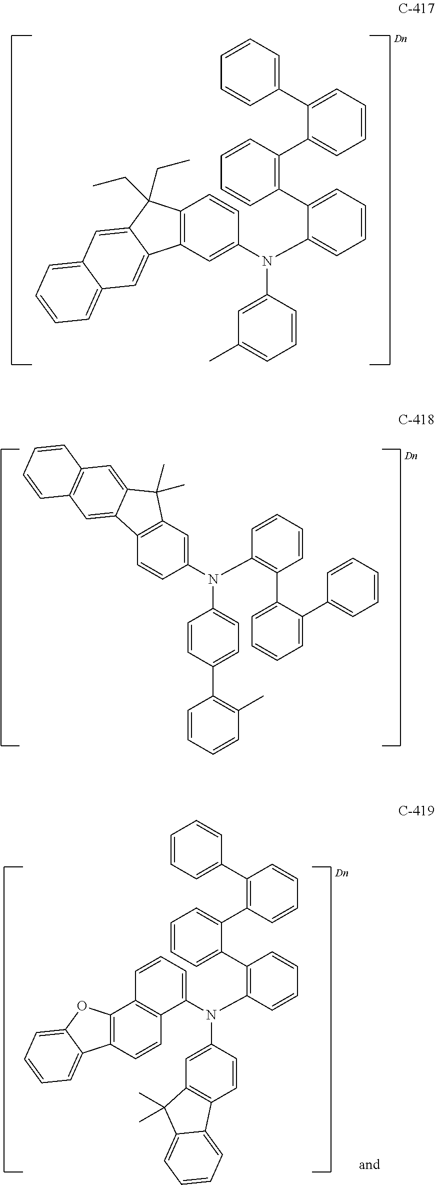

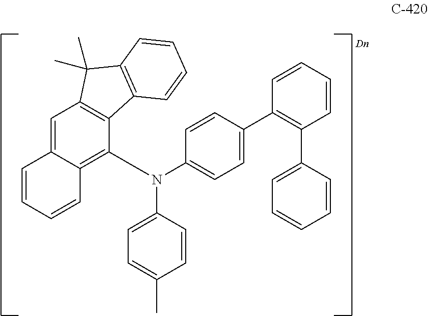

In the compounds above, Dn represents that n number of hydrogens are replaced with deuterium, and n represents an integer of 1 or more, which is an integer of 1 to the maximum number of hydrogens in the compound.

Examples of the production method for the compound represented by any one of Formula 1 or Formula 1-1 to 1-11 according to the present disclosure are shown in the following Reaction Scheme 1 and Reaction Schemes 1-1 to 1-4, but are not limited thereto. It may be produced by way of synthetic methods known to one skilled in the art.

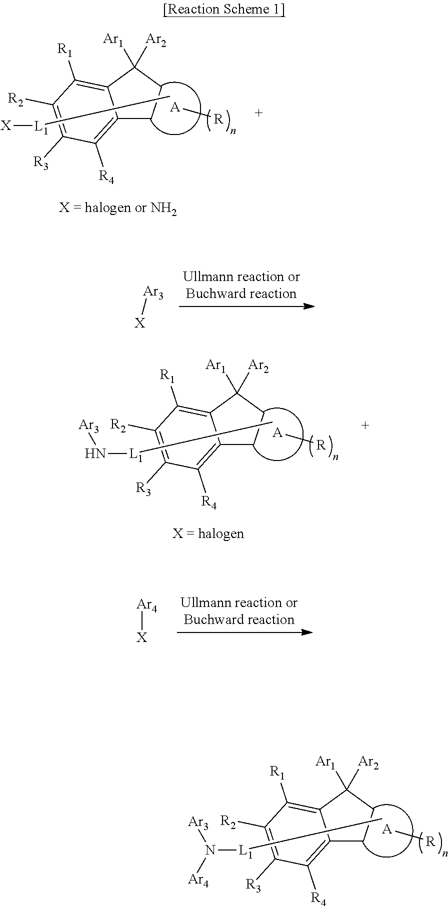

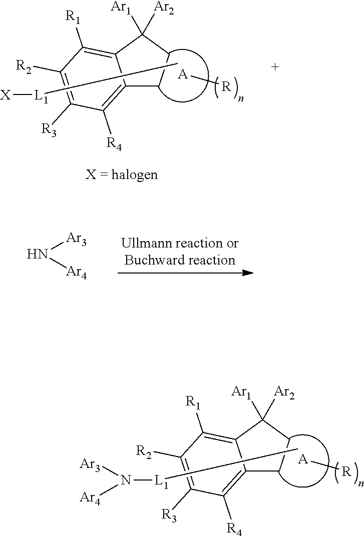

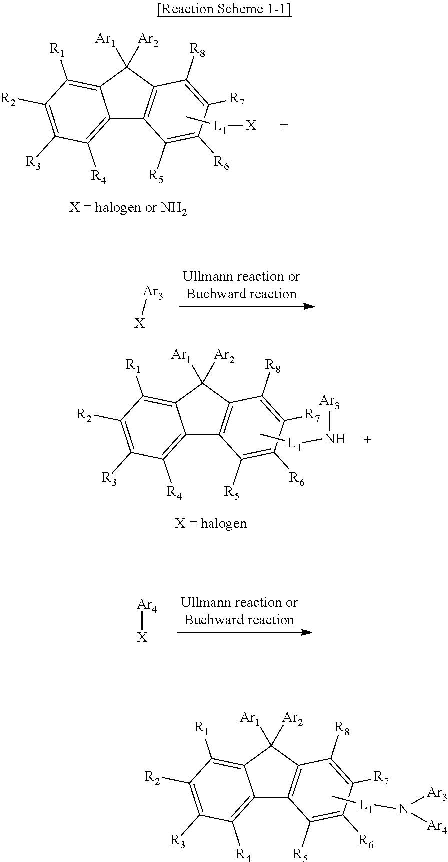

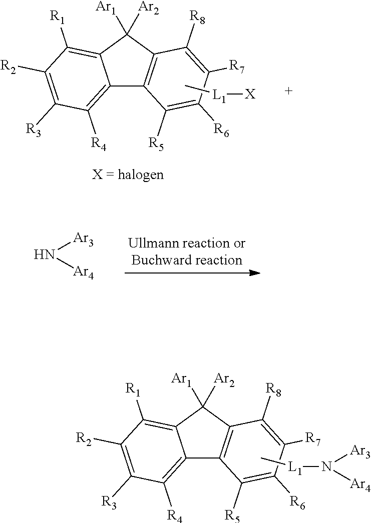

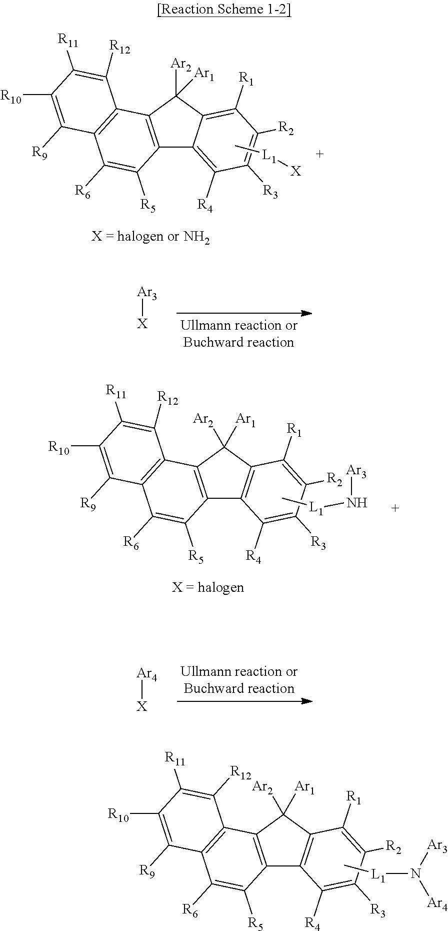

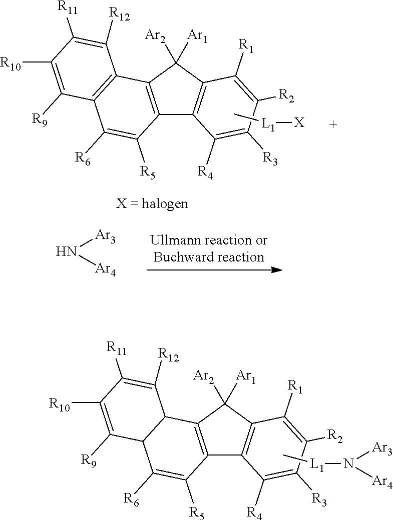

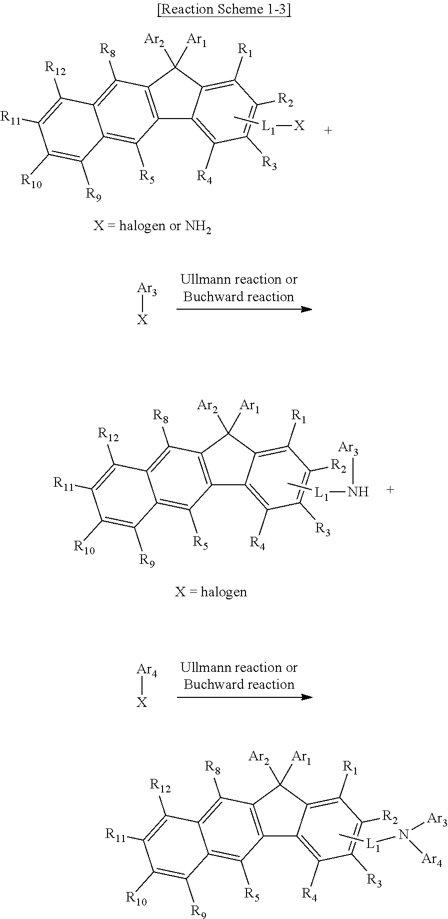

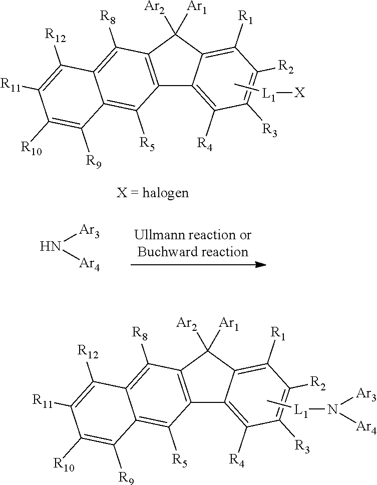

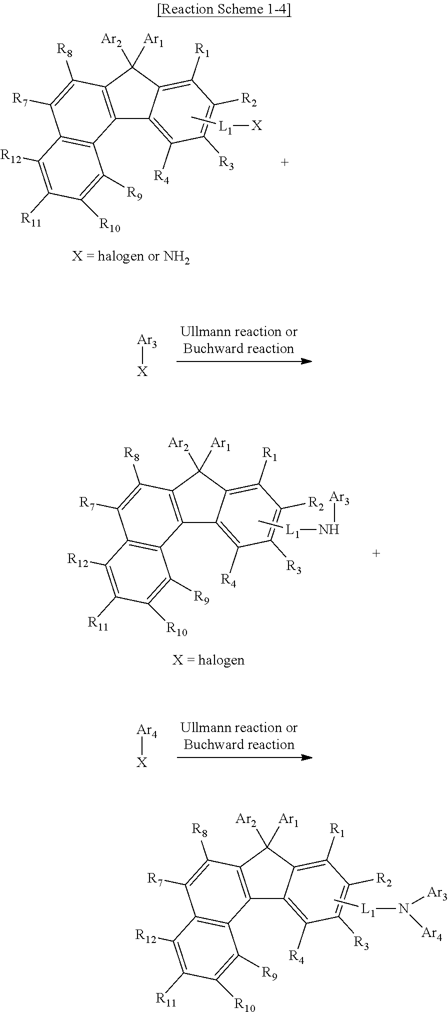

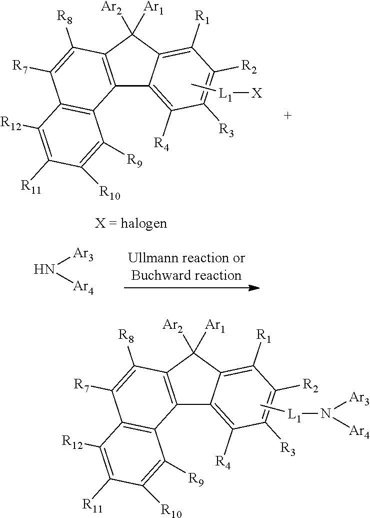

In the above Reaction Schemes, R and n are as defined in Formula 1, and R₁ to R₁₂, L₁, and Ar₁ to Ar₄ are as defined in each of Formulas 1-1 to 1-11.

Although illustrative synthesis examples of the compound represented by Formulas 1-1 to 1-11 are described above, one skilled in the art will be able to readily understand that all of these are based on a Buchwald-Hartwig cross-coupling reaction, an N-arylation reaction, an H-mont-mediated etherification reaction, a Miyaura borylation reaction, a Suzuki cross-coupling reaction, an intramolecular acid-induced cyclization reaction, a Pd(II)-catalyzed oxidative cyclization reaction, a Grignard reaction, a Heck reaction, a cyclic dehydration reaction, an SN₁ substitution reaction, an SN₂ substitution reaction, a phosphine-mediated reductive cyclization reaction, etc., and the above reaction proceeds even when substituents which are defined in Formulas 1-1 to 1-11 but not specified in the specific synthesis example, are bonded.

In the organic electroluminescent device, the light-emitting layer emits any one color of blue, green, or red, with a proviso that when the light-emitting layer emits the blue or green color, the case of R₂ or R₇ of Formula 1-1 is -L₁-NAr₃Ar₄ is excluded.

In the organic electroluminescent device, the third hole transport layer comprises the compound represented by the following Formula 2.

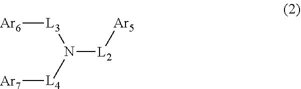

In Formula 2,

L₂ to L₄ each independently represent a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene. According to one embodiment of the present disclosure, L₂ to L₄ each independently may represent a single bond, a substituted or unsubstituted (C5-C25)alkylene, or a substituted or unsubstituted (C6-C25)arylene. According to another embodiment of the present disclosure, L₂ to L₄ each independently may represent a single bond, or a substituted or unsubstituted (C6-C25)arylene. For example, L₂ to L₄ each independently may represent a single bond, a phenylene substituted or unsubstituted with deuterium, a biphenylene, a terphenylene, or a naphthylene, and these may be substituted with at least one deuterium.

Ar₅ to Ar₇ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di(C1-C30)alkylamino, a substituted or unsubstituted mono- or di(C6-C30)arylamino, or a substituted or unsubstituted (C1-C30)alkyl(C6-C30)arylamino. According to one embodiment of the present disclosure, Ar₅ to Ar₇ each independently may represent hydrogen, deuterium, a halogen, a cyano, a substituted or unsubstituted (C1-C25)alkyl, a substituted or unsubstituted (C6-C25)aryl, a substituted or unsubstituted (3- to 25-membered)heteroaryl, a substituted or unsubstituted (C3-C25)cycloalkyl, a substituted or unsubstituted (C1-C25)alkoxy, a substituted or unsubstituted tri(C1-C25)alkylsilyl, a substituted or unsubstituted di(C1-C25)alkyl(C6-C25)arylsilyl, a substituted or unsubstituted tri(C6-C25)arylsilyl. For example, Ar₅ to Ar₇ each independently may represent a phenyl, a biphenyl, a naphthyl, a dibenzofuranyl, a dibenzothiophenyl, a dibenzoselenophenyl, a benzonaphthofuranyl, benzonaphthothiophenyl, a dimethylfluorenyl, a diethylfluorenyl, a diphenylfluorenyl, a diphenylbenzofluorenyl, a spirobifluorenyl, a carbazolyl substituted or unsubstituted with a phenyl or a naphthyl, a phenanthrenyl substituted or unsubstituted with a phenyl or a naphthyl, an o-terphenyl, a m-terphenyl, a p-terphenyl, an adamantyl or any one of the following Formulas 2-1 to 2-6, and these may be substituted with at least one deuterium.

With a proviso that the case where L₂ to L₄ are all single bonds, and Ar₅ to Ar₇ are all hydrogen is excluded.

In the organic electroluminescent device, the Ar₅ in Formula 2 is represented by any one of the following Formulas 2-1 to 2-6.

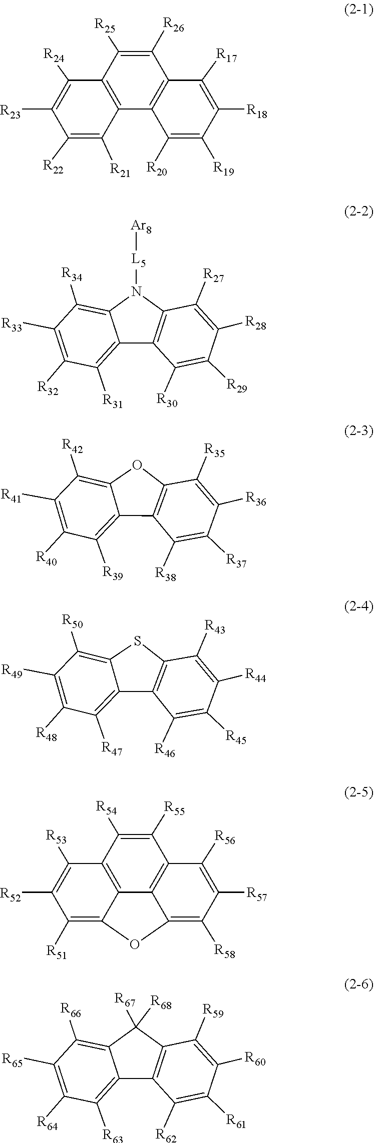

In Formulas 2-1 to 2-6,

L₅ represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene. According to one embodiment of the present disclosure, L₅ may represent a single bond, a substituted or unsubstituted (C1-C30)alkylene, or a substituted or unsubstituted (C6-C30)arylene. According to another embodiment of the present disclosure, L₅ may represent a single bond, a substituted or unsubstituted (C1-C20)alkylene, or a substituted or unsubstituted (C6-C20)arylene. For example, L₅ may represent a single bond, or a phenylene, and these may be substituted with at least one deuterium.

Ar₈ links to L₂ of Formula 2, or represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di(C1-C30)alkylamino, a substituted or unsubstituted mono- or di(C6-C30)arylamino, or a substituted or unsubstituted (C1-C30)alkyl(C6-C30)arylamino. According to one embodiment of the present disclosure, Ar₈ may link to L₂ of Formula 2, or may represent hydrogen, deuterium, a substituted or unsubstituted (C1-C30)alkyl, a substituted or unsubstituted (C6-C30)aryl. According to another embodiment of the present disclosure, Ar₈ may link to L₂ of Formula 2, or may represent hydrogen, a substituted or unsubstituted (C1-C20)alkyl, a substituted or unsubstituted (C6-C20)aryl. For example, Ar₈ may link to L₂ of Formula 2, or may represent a phenyl or a naphthyl, and these may be substituted with at least one deuterium.

R₁₇ to R₆₈ are linked to L₂ of Formula 2, or each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di(C1-C30)alkylamino, a substituted or unsubstituted mono- or di(C6-C30)arylamino, or a substituted or unsubstituted (C1-C30)alkyl(C6-C30)arylamino; or may be linked to an adjacent substituent(s) to form a ring(s). According to one embodiment of the present disclosure, R₁₇ to R₆₈ may be linked to L₂ of Formula 2, or each independently may represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy; or may be linked to an adjacent substituent(s) to form a ring(s). According to another embodiment of the present disclosure, R₁₇ to R₆₈ may be linked to L₂ of Formula 2, or each independently may represent hydrogen, deuterium, a substituted or unsubstituted (C6-C25)aryl, or a substituted or unsubstituted (3- to 25-membered)heteroaryl. For example, R₁₇ to R₆₈ may be linked to L₂ of Formula 2, or each independently may represent hydrogen, deuterium, a phenyl, or a naphthyl.

With a proviso that at least one of Ar₈ and R₁₇ to R₆₈ is linked to L₂ of Formula 2.

In the organic electroluminescent device, Formula 2-3 is represented by any one of the following Formulas 2-3-a to 2-3-c, or Formula 2-4 is represented by any one of the following Formulas 2-4-a to 2-4-c.

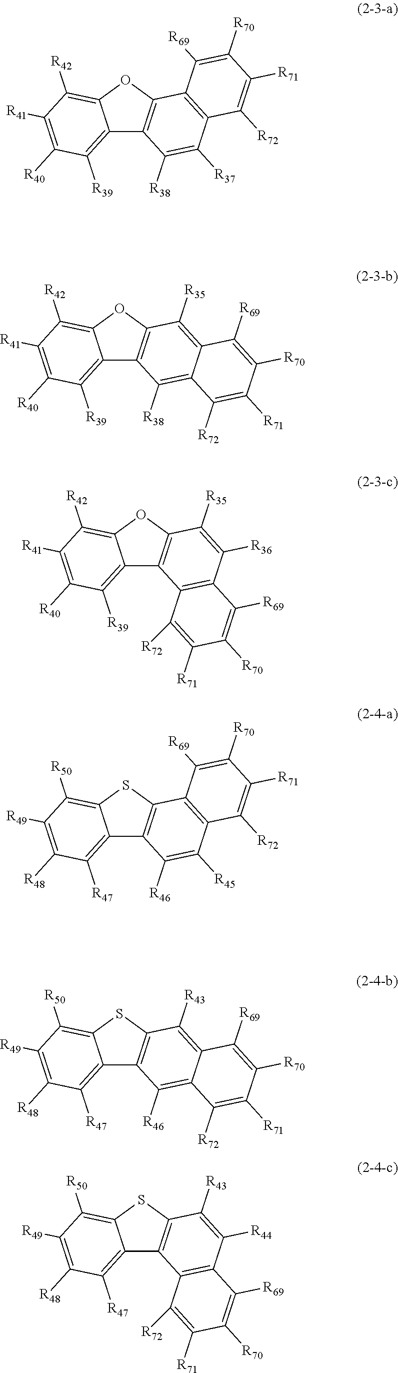

In Formulas 2-3-a to 2-3-c and Formulas 2-4-a to 2-4-c,

R₃₅ to R₅₀ are as defined in Formulas 2-1 to 2-6.

R₆₉ to R₇₂ are linked to L₂ of Formula 2, or each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di(C1-C30)alkylamino, a substituted or unsubstituted mono- or di(C6-C30)arylamino, or a substituted or unsubstituted (C1-C30)alkyl(C6-C30)arylamino; or may be linked to an adjacent substituent(s) to form a ring(s). According to one embodiment of the present disclosure, R₆₉ to R₇₂ may be linked to L₂ of Formula 2, or each independently may represent hydrogen, deuterium, a substituted or unsubstituted (C1-C30)alkyl, a substituted or unsubstituted (C6-C30)aryl, or a substituted or unsubstituted (3- to 30-membered)heteroaryl. For example, R₆₉ to R₇₂ may be linked to L₂ of Formula 2, and each independently may represent hydrogen.

With a proviso that at least one of R₃₅ to R₅₀, and R₆₉ to R₇₂ is linked to L₂ of Formula 2.

The compound represented by Formula 2 may be at least one selected from the following compounds, but is not limited thereto.

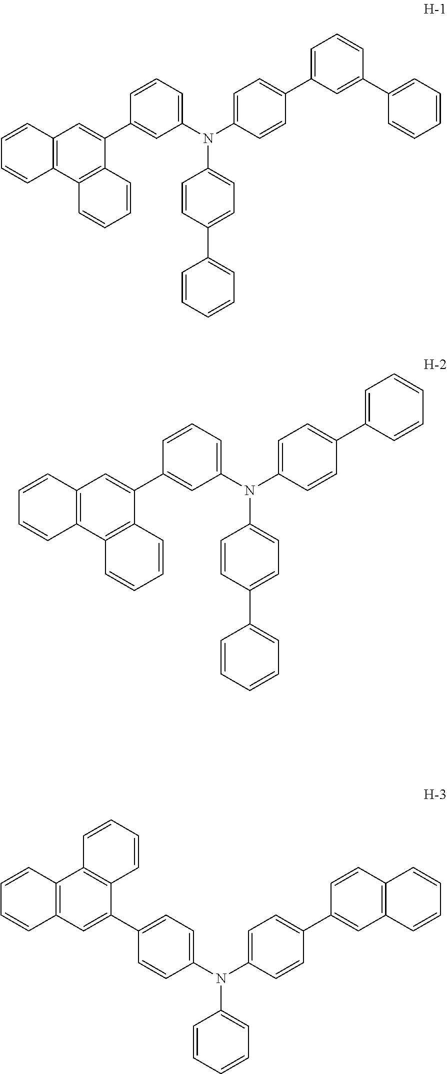 [SMILES: c1ccc(-c2ccc(N(c3ccc(-c4cccc(-c5ccccc5)c4)cc3)c3cccc(-c4cc5ccccc5c5ccccc45)c3)cc2)cc1 ; c1ccc(-c2ccc(N(c3ccc(-c4ccccc4)cc3)c3cccc(-c4cc5ccccc5c5ccccc45)c3)cc2)cc1 ; c1ccc(N(c2ccc(-c3ccc4ccccc4c3)cc2)c2ccc(-c3cc4ccccc4c4ccccc34)cc2)cc1]

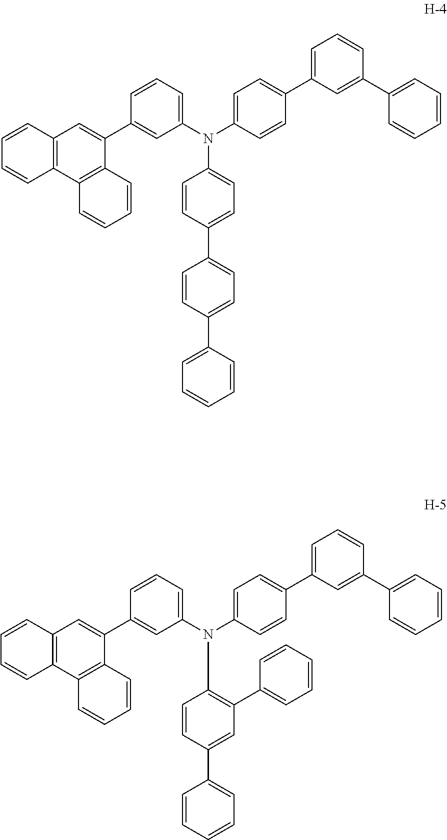 [SMILES: c1ccc(-c2ccc(-c3ccc(N(c4ccc(-c5cccc(-c6ccccc6)c5)cc4)c4cccc(-c5cc6ccccc6c6ccccc56)c4)cc3)cc2)cc1 ; c1ccc(-c2cccc(-c3ccc(N(c4cccc(-c5cc6ccccc6c6ccccc56)c4)c4ccc(-c5ccccc5)cc4-c4ccccc4)cc3)c2)cc1]

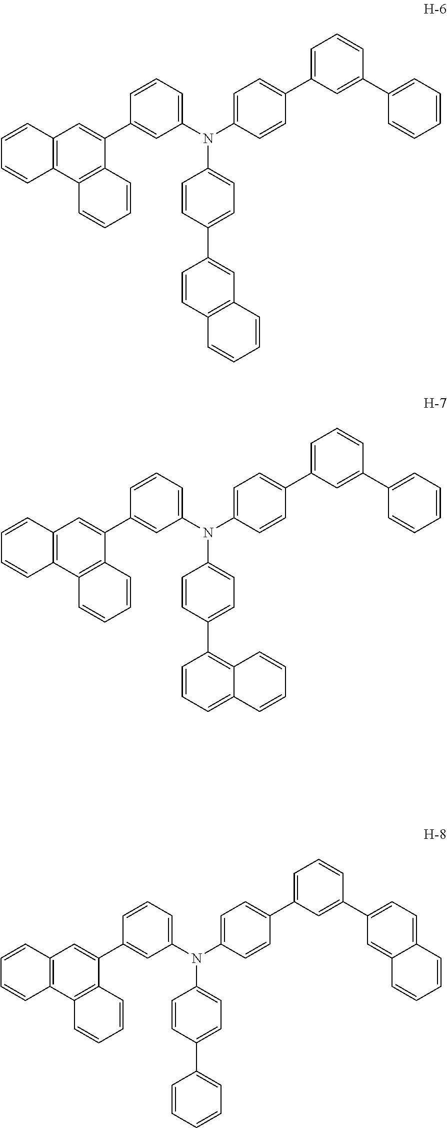 [SMILES: c1ccc(-c2cccc(-c3ccc(N(c4ccc(-c5ccc6ccccc6c5)cc4)c4cccc(-c5cc6ccccc6c6ccccc56)c4)cc3)c2)cc1 ; c1ccc(-c2cccc(-c3ccc(N(c4ccc(-c5cccc6ccccc56)cc4)c4cccc(-c5cc6ccccc6c6ccccc56)c4)cc3)c2)cc1 ; c1ccc(-c2ccc(N(c3ccc(-c4cccc(-c5ccc6ccccc6c5)c4)cc3)c3cccc(-c4cc5ccccc5c5ccccc45)c3)cc2)cc1]

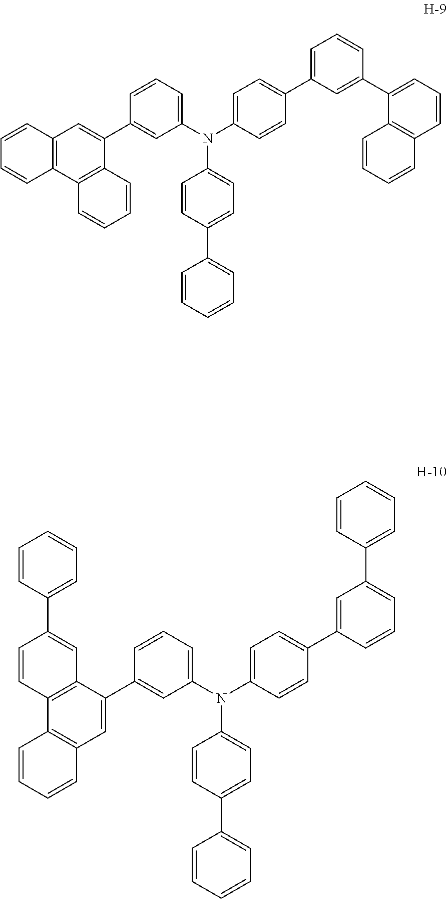 [SMILES: c1ccc(-c2ccc(N(c3ccc(-c4cccc(-c5cccc6ccccc56)c4)cc3)c3cccc(-c4cc5ccccc5c5ccccc45)c3)cc2)cc1 ; c1ccc(-c2ccc(N(c3ccc(-c4cccc(-c5ccccc5)c4)cc3)c3cccc(-c4cc5ccccc5c5ccc(-c6ccccc6)cc45)c3)cc2)cc1]

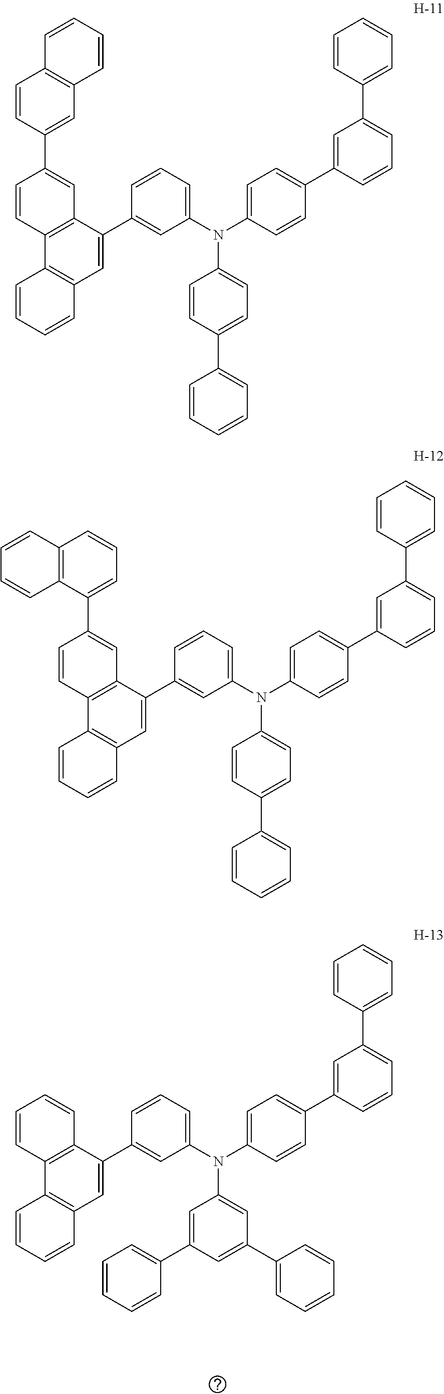 [SMILES: c1ccc(-c2ccc(N(c3ccc(-c4cccc(-c5ccccc5)c4)cc3)c3cccc(-c4cc5ccccc5c5ccc(-c6ccc7ccccc7c6)cc45)c3)cc2)cc1 ; c1ccc(-c2ccc(N(c3ccc(-c4cccc(-c5ccccc5)c4)cc3)c3cccc(-c4cc5ccccc5c5ccc(-c6cccc7ccccc67)cc45)c3)cc2)cc1 ; c1ccc(-c2cccc(-c3ccc(N(c4cc(-c5ccccc5)cc(-c5ccccc5)c4)c4cccc(-c5cc6ccccc6c6ccccc56)c4)cc3)c2)cc1]

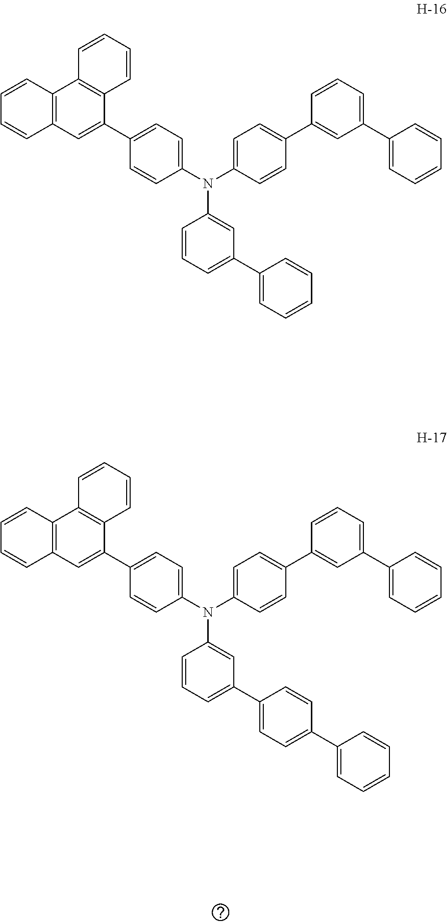 [SMILES: c1ccc(-c2cccc(-c3ccc(N(c4ccc(-c5cc6ccccc6c6ccccc56)cc4)c4cccc(-c5ccccc5)c4)cc3)c2)cc1 ; c1ccc(-c2ccc(-c3cccc(N(c4ccc(-c5cccc(-c6ccccc6)c5)cc4)c4ccc(-c5cc6ccccc6c6ccccc56)cc4)c3)cc2)cc1]

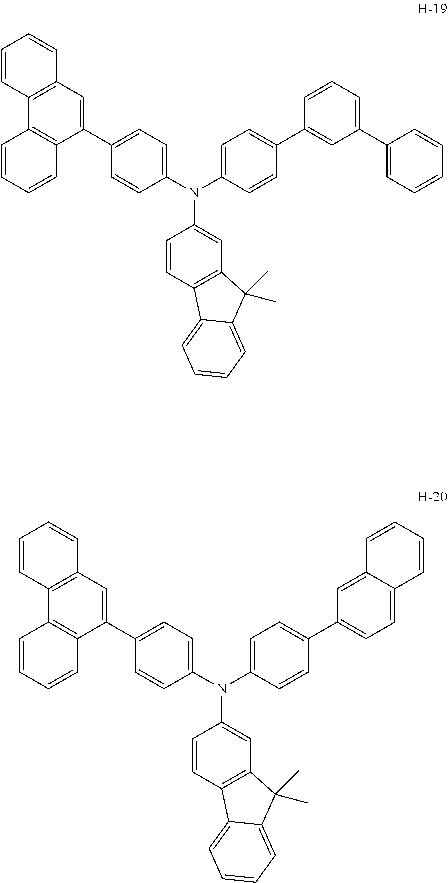 [SMILES: CC1(C)c2ccccc2-c2ccc(N(c3ccc(-c4cccc(-c5ccccc5)c4)cc3)c3ccc(-c4cc5ccccc5c5ccccc45)cc3)cc21 ; CC1(C)c2ccccc2-c2ccc(N(c3ccc(-c4ccc5ccccc5c4)cc3)c3ccc(-c4cc5ccccc5c5ccccc45)cc3)cc21]

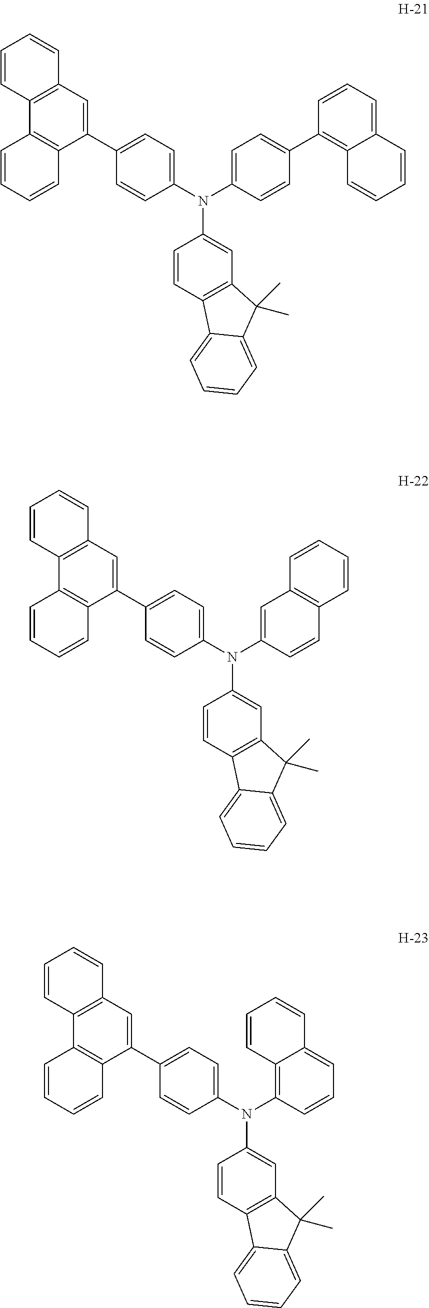 [SMILES: CC1(C)c2ccccc2-c2ccc(N(c3ccc(-c4cccc5ccccc45)cc3)c3ccc(-c4cc5ccccc5c5ccccc45)cc3)cc21 ; CC1(C)c2ccccc2-c2ccc(N(c3ccc(-c4cc5ccccc5c5ccccc45)cc3)c3ccc4ccccc4c3)cc21 ; CC1(C)c2ccccc2-c2ccc(N(c3ccc(-c4cc5ccccc5c5ccccc45)cc3)c3cccc4ccccc34)cc21]

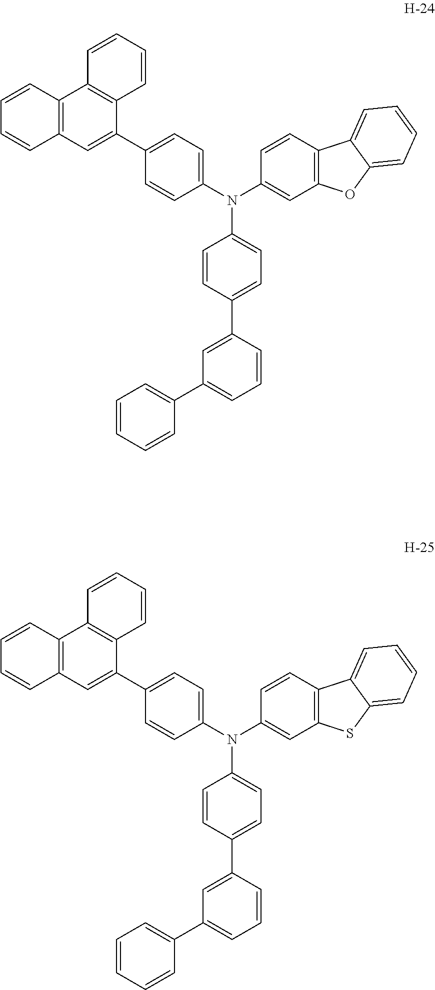 [SMILES: c1ccc(-c2cccc(-c3ccc(N(c4ccc(-c5cc6ccccc6c6ccccc56)cc4)c4ccc5c(c4)oc4ccccc45)cc3)c2)cc1 ; c1ccc(-c2cccc(-c3ccc(N(c4ccc(-c5cc6ccccc6c6ccccc56)cc4)c4ccc5c(c4)sc4ccccc45)cc3)c2)cc1]

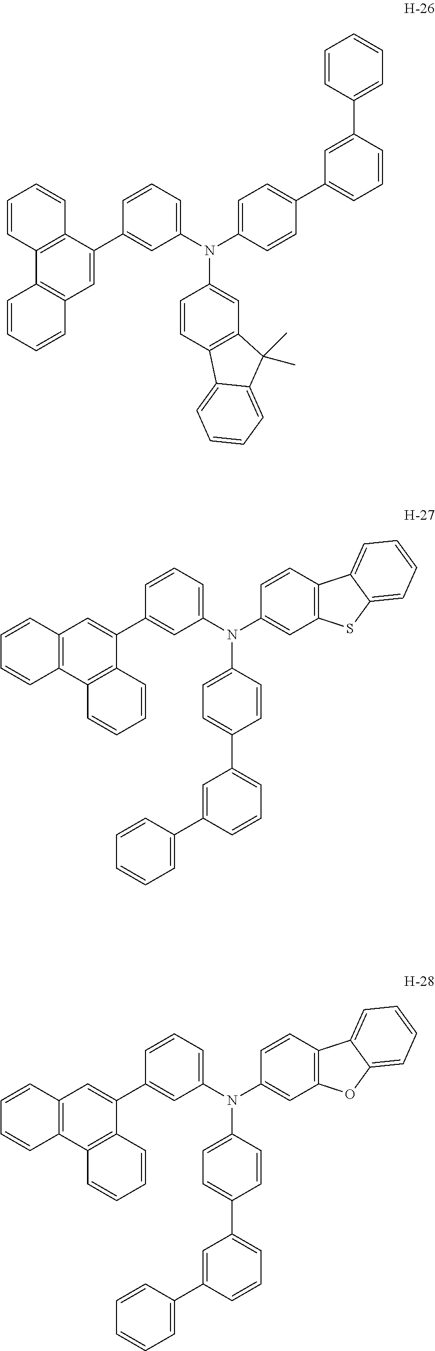 [SMILES: CC1(C)c2ccccc2-c2ccc(N(c3ccc(-c4cccc(-c5ccccc5)c4)cc3)c3cccc(-c4cc5ccccc5c5ccccc45)c3)cc21 ; c1ccc(-c2cccc(-c3ccc(N(c4cccc(-c5cc6ccccc6c6ccccc56)c4)c4ccc5c(c4)sc4ccccc45)cc3)c2)cc1 ; c1ccc(-c2cccc(-c3ccc(N(c4cccc(-c5cc6ccccc6c6ccccc56)c4)c4ccc5c(c4)oc4ccccc45)cc3)c2)cc1]

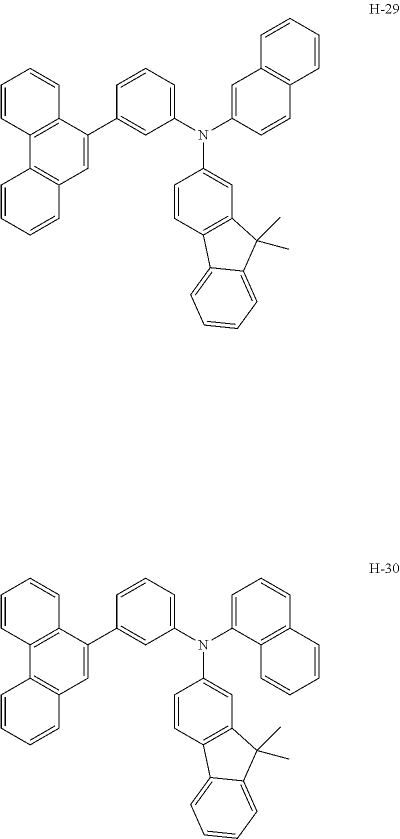 [SMILES: CC1(C)c2ccccc2-c2ccc(N(c3cccc(-c4cc5ccccc5c5ccccc45)c3)c3ccc4ccccc4c3)cc21 ; CC1(C)c2ccccc2-c2ccc(N(c3cccc(-c4cc5ccccc5c5ccccc45)c3)c3cccc4ccccc34)cc21]

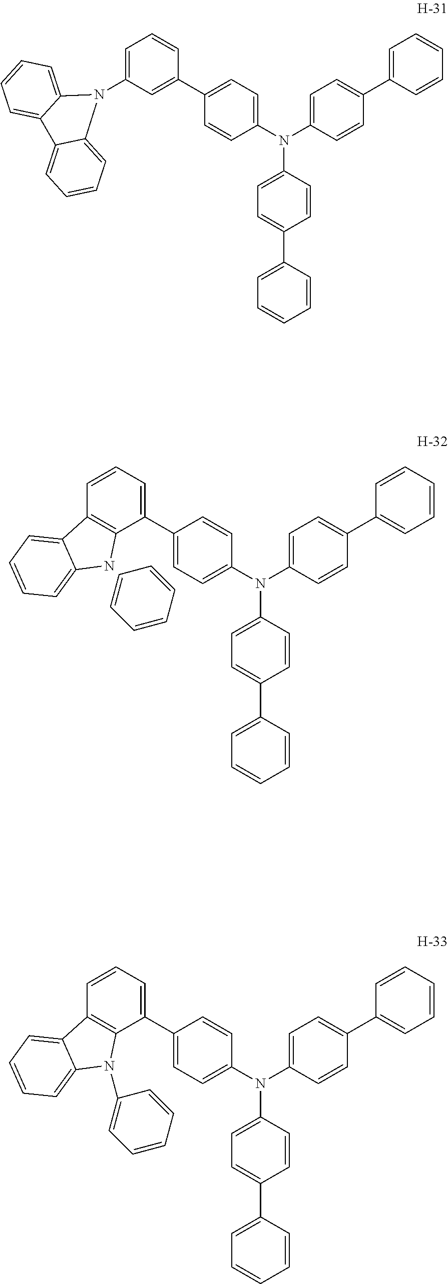

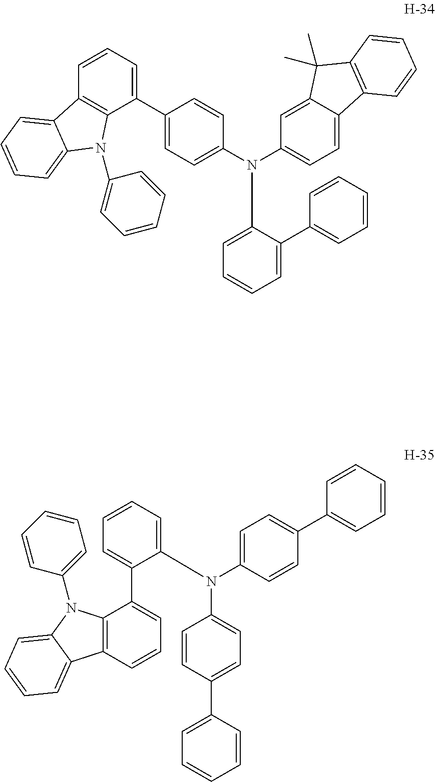 [SMILES: CC1(C)c2ccccc2-c2ccc(N(c3ccc(-c4cccc5c6ccccc6n(-c6ccccc6)c45)cc3)c3ccccc3-c3ccccc3)cc21 ; c1ccc(-c2ccc(N(c3ccc(-c4ccccc4)cc3)c3ccccc3-c3cccc4c5ccccc5n(-c5ccccc5)c34)cc2)cc1]

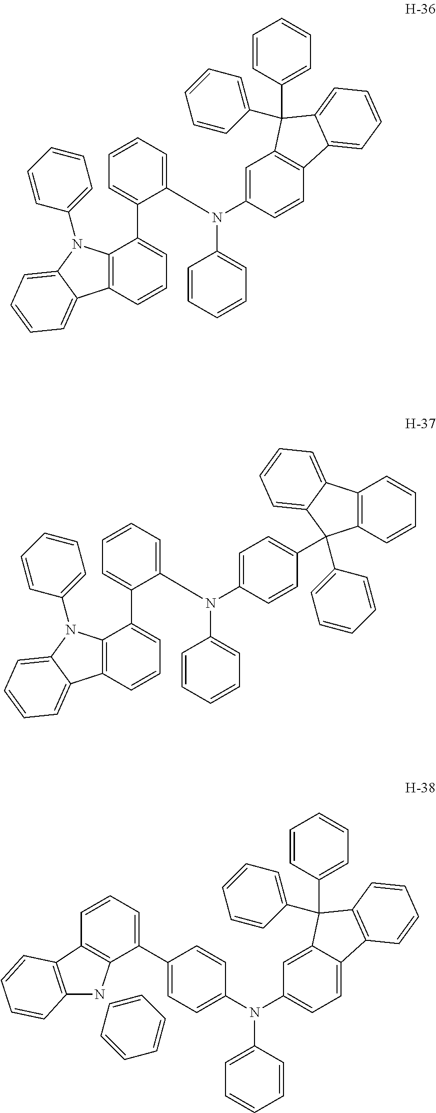

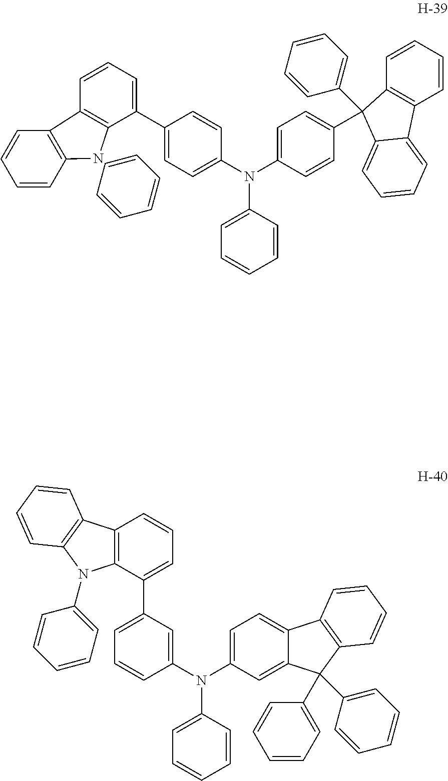

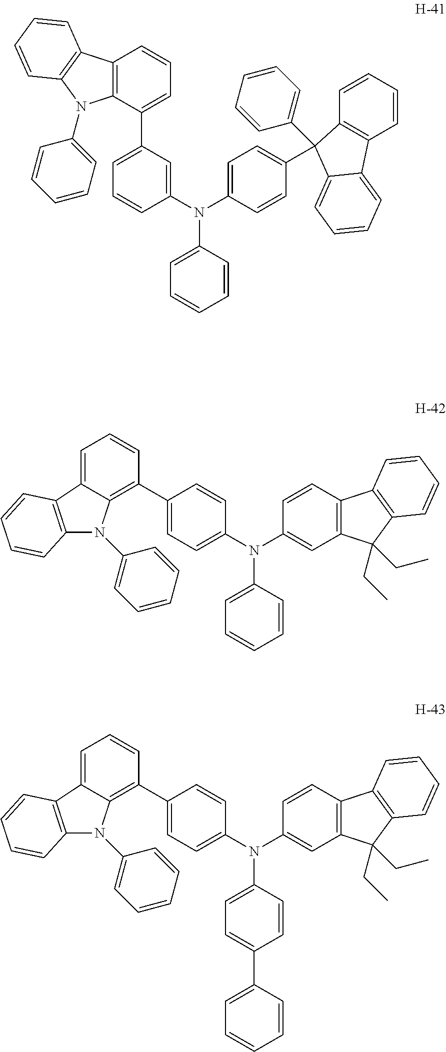 [SMILES: c1ccc(N(c2ccc(C3(c4ccccc4)c4ccccc4-c4ccccc43)cc2)c2cccc(-c3cccc4c5ccccc5n(-c5ccccc5)c34)c2)cc1 ; CCC1(CC)c2ccccc2-c2ccc(N(c3ccccc3)c3ccc(-c4cccc5c6ccccc6n(-c6ccccc6)c45)cc3)cc21 ; CCC1(CC)c2ccccc2-c2ccc(N(c3ccc(-c4ccccc4)cc3)c3ccc(-c4cccc5c6ccccc6n(-c6ccccc6)c45)cc3)cc21]

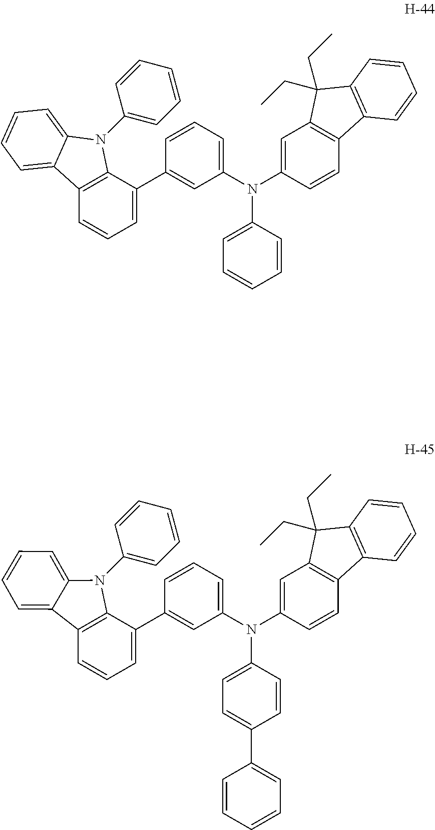 [SMILES: CCC1(CC)c2ccccc2-c2ccc(N(c3ccccc3)c3cccc(-c4cccc5c6ccccc6n(-c6ccccc6)c45)c3)cc21 ; CCC1(CC)c2ccccc2-c2ccc(N(c3ccc(-c4ccccc4)cc3)c3cccc(-c4cccc5c6ccccc6n(-c6ccccc6)c45)c3)cc21]

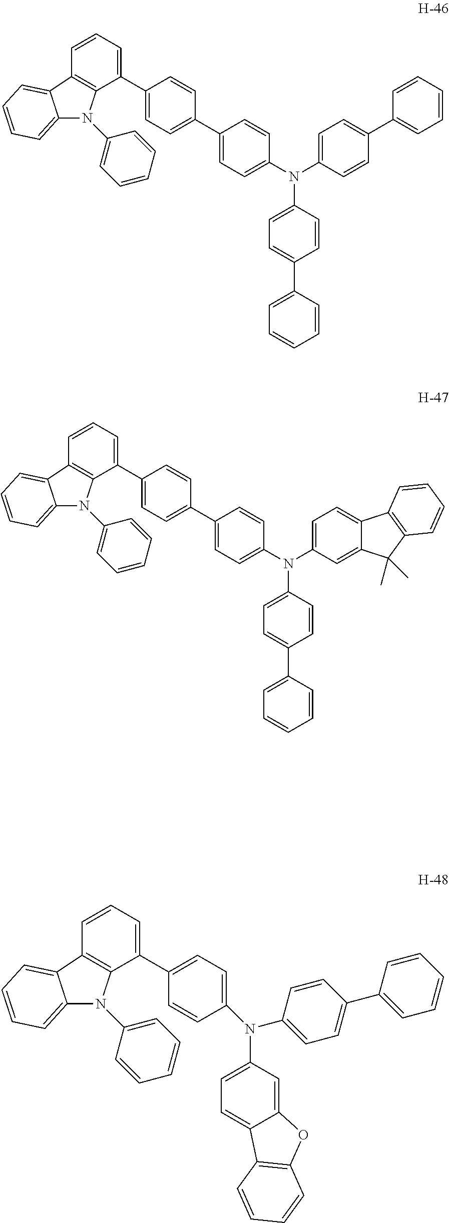 [SMILES: c1ccc(-c2ccc(N(c3ccc(-c4ccccc4)cc3)c3ccc(-c4ccc(-c5cccc6c7ccccc7n(-c7ccccc7)c56)cc4)cc3)cc2)cc1 ; CC1(C)c2ccccc2-c2ccc(N(c3ccc(-c4ccccc4)cc3)c3ccc(-c4ccc(-c5cccc6c7ccccc7n(-c7ccccc7)c56)cc4)cc3)cc21 ; c1ccc(-c2ccc(N(c3ccc(-c4cccc5c6ccccc6n(-c6ccccc6)c45)cc3)c3ccc4c(c3)oc3ccccc34)cc2)cc1]

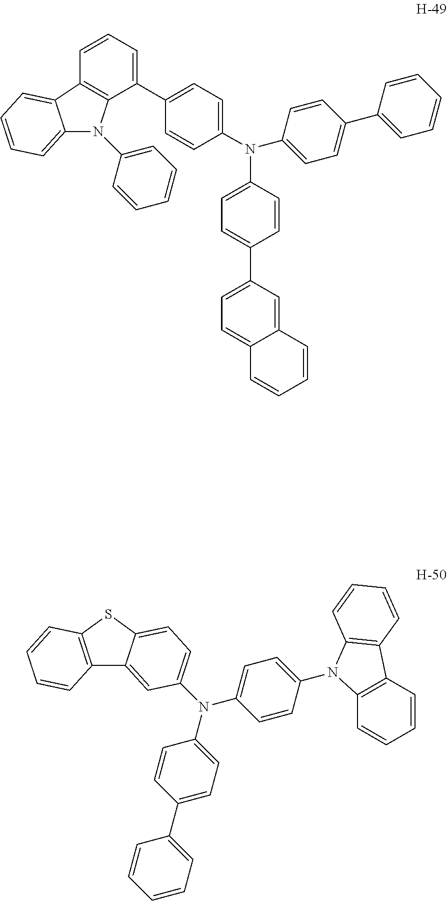 [SMILES: c1ccc(-c2ccc(N(c3ccc(-c4ccc5ccccc5c4)cc3)c3ccc(-c4cccc5c6ccccc6n(-c6ccccc6)c45)cc3)cc2)cc1 ; c1ccc(-c2ccc(N(c3ccc(-n4c5ccccc5c5ccccc54)cc3)c3ccc4sc5ccccc5c4c3)cc2)cc1]

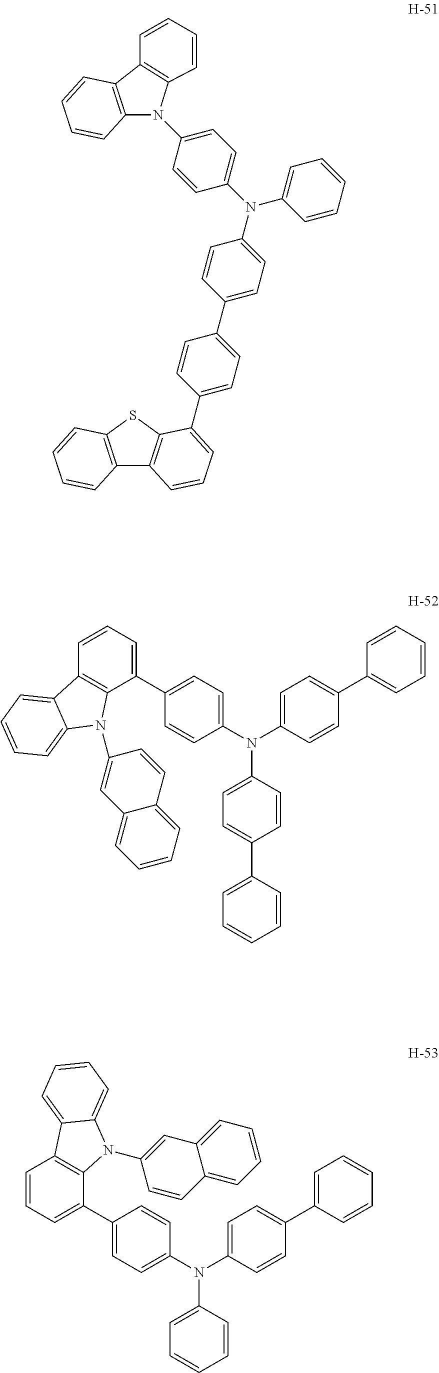 [SMILES: c1ccc(N(c2ccc(-c3ccc(-c4cccc5c4sc4ccccc45)cc3)cc2)c2ccc(-n3c4ccccc4c4ccccc43)cc2)cc1 ; c1ccc(-c2ccc(N(c3ccc(-c4ccccc4)cc3)c3ccc(-c4cccc5c6ccccc6n(-c6ccc7ccccc7c6)c45)cc3)cc2)cc1 ; c1ccc(-c2ccc(N(c3ccccc3)c3ccc(-c4cccc5c6ccccc6n(-c6ccc7ccccc7c6)c45)cc3)cc2)cc1]

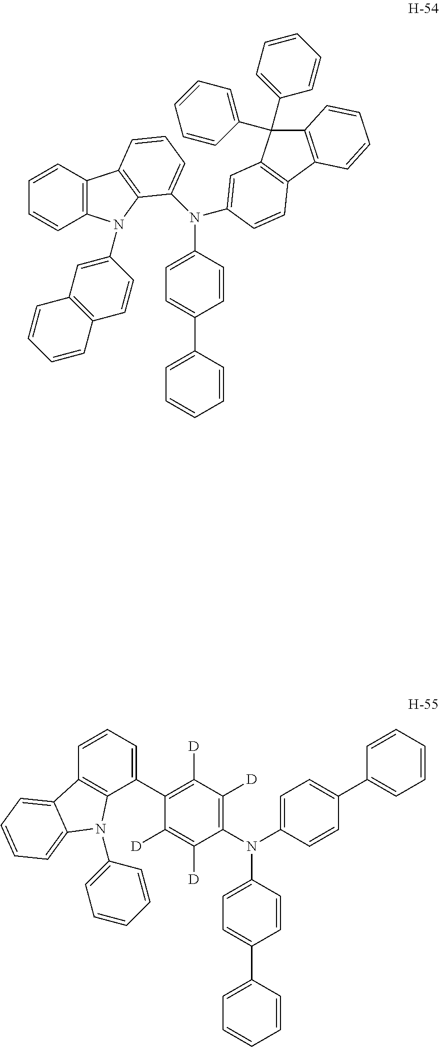 [SMILES: c1ccc(-c2ccc(N(c3ccc4c(c3)C(c3ccccc3)(c3ccccc3)c3ccccc3-4)c3cccc4c5ccccc5n(-c5ccc6ccccc6c5)c34)cc2)cc1 ; [2H]c1c([2H])c(N(c2ccc(-c3ccccc3)cc2)c2ccc(-c3ccccc3)cc2)c([2H])c([2H])c1-c1cccc2c3ccccc3n(-c3ccccc3)c12]

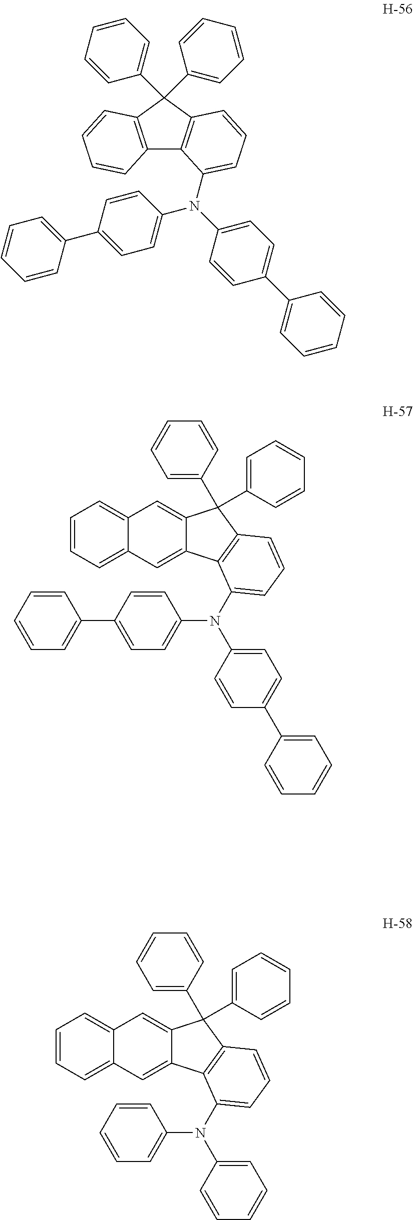 [SMILES: c1ccc(-c2ccc(N(c3ccc(-c4ccccc4)cc3)c3cccc4c3-c3ccccc3C4(c3ccccc3)c3ccccc3)cc2)cc1 ; c1ccc(-c2ccc(N(c3ccc(-c4ccccc4)cc3)c3cccc4c3-c3cc5ccccc5cc3C4(c3ccccc3)c3ccccc3)cc2)cc1 ; c1ccc(N(c2ccccc2)c2cccc3c2-c2cc4ccccc4cc2C3(c2ccccc2)c2ccccc2)cc1]

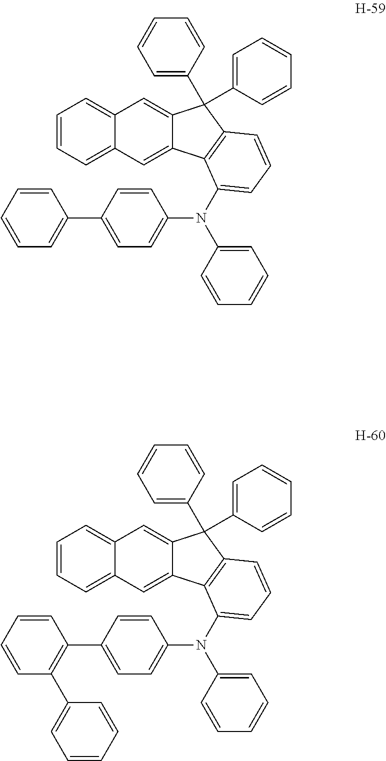 [SMILES: c1ccc(-c2ccc(N(c3ccccc3)c3cccc4c3-c3cc5ccccc5cc3C4(c3ccccc3)c3ccccc3)cc2)cc1 ; c1ccc(-c2ccccc2-c2ccc(N(c3ccccc3)c3cccc4c3-c3cc5ccccc5cc3C4(c3ccccc3)c3ccccc3)cc2)cc1]

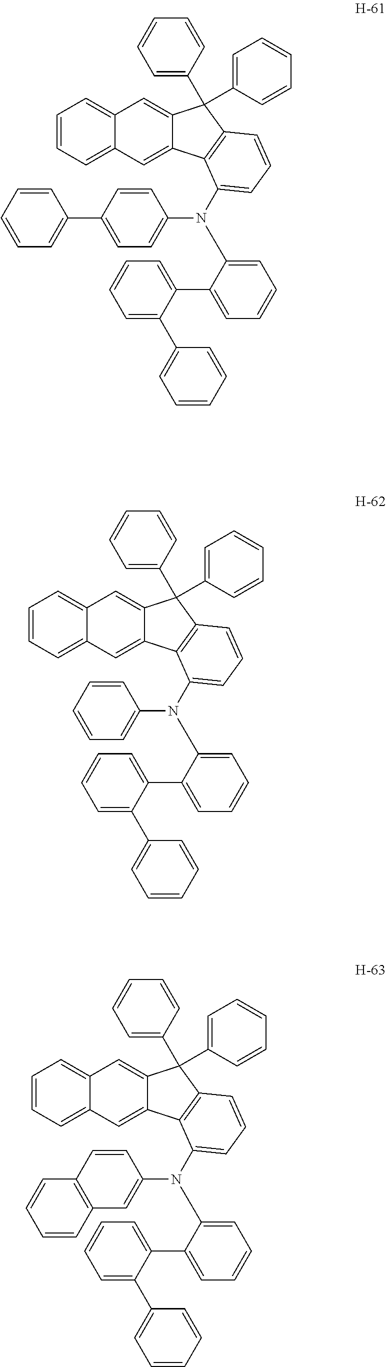 [SMILES: c1ccc(-c2ccc(N(c3ccccc3-c3ccccc3-c3ccccc3)c3cccc4c3-c3cc5ccccc5cc3C4(c3ccccc3)c3ccccc3)cc2)cc1 ; c1ccc(-c2ccccc2-c2ccccc2N(c2ccccc2)c2cccc3c2-c2cc4ccccc4cc2C3(c2ccccc2)c2ccccc2)cc1 ; c1ccc(-c2ccccc2-c2ccccc2N(c2ccc3ccccc3c2)c2cccc3c2-c2cc4ccccc4cc2C3(c2ccccc2)c2ccccc2)cc1]

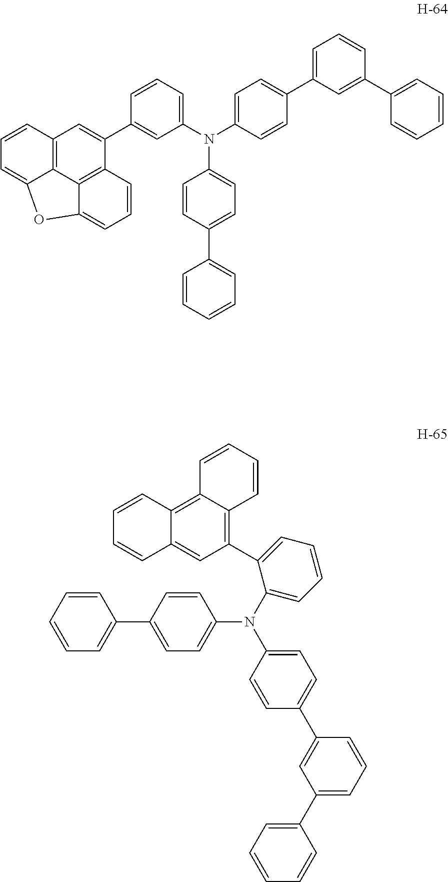 [SMILES: c1ccc(-c2ccc(N(c3ccc(-c4cccc(-c5ccccc5)c4)cc3)c3cccc(-c4cc5cccc6oc7cccc4c7c56)c3)cc2)cc1 ; c1ccc(-c2ccc(N(c3ccc(-c4cccc(-c5ccccc5)c4)cc3)c3ccccc3-c3cc4ccccc4c4ccccc34)cc2)cc1]

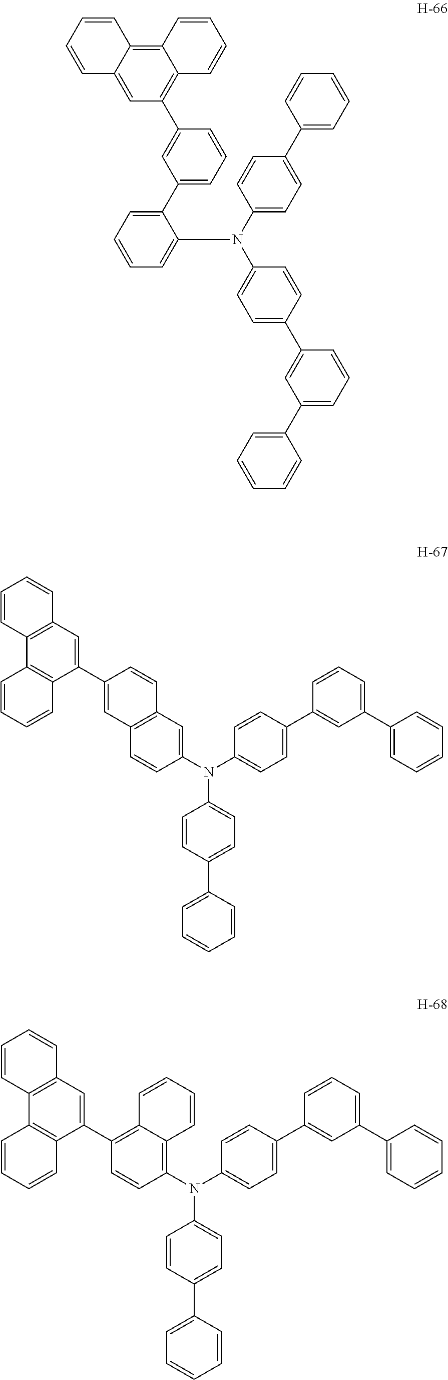 [SMILES: c1ccc(-c2ccc(N(c3ccc(-c4cccc(-c5ccccc5)c4)cc3)c3ccccc3-c3cccc(-c4cc5ccccc5c5ccccc45)c3)cc2)cc1 ; c1ccc(-c2ccc(N(c3ccc(-c4cccc(-c5ccccc5)c4)cc3)c3ccc4cc(-c5cc6ccccc6c6ccccc56)ccc4c3)cc2)cc1 ; c1ccc(-c2ccc(N(c3ccc(-c4cccc(-c5ccccc5)c4)cc3)c3ccc(-c4cc5ccccc5c5ccccc45)c4ccccc34)cc2)cc1]

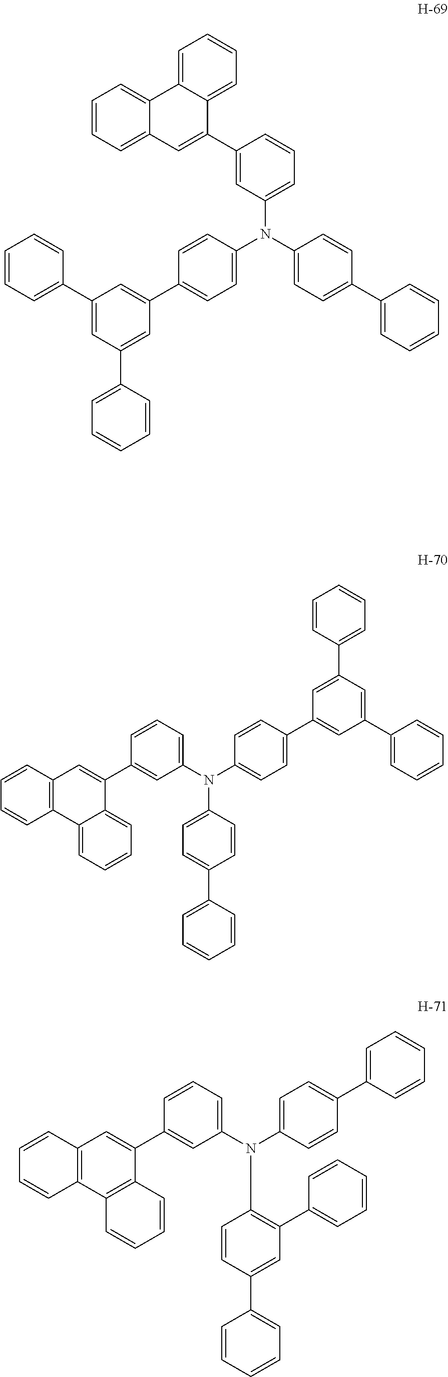 [SMILES: c1ccc(-c2ccc(N(c3ccc(-c4cc(-c5ccccc5)cc(-c5ccccc5)c4)cc3)c3cccc(-c4cc5ccccc5c5ccccc45)c3)cc2)cc1 ; c1ccc(-c2ccc(N(c3ccc(-c4cc(-c5ccccc5)cc(-c5ccccc5)c4)cc3)c3cccc(-c4cc5ccccc5c5ccccc45)c3)cc2)cc1 ; c1ccc(-c2ccc(N(c3cccc(-c4cc5ccccc5c5ccccc45)c3)c3ccc(-c4ccccc4)cc3-c3ccccc3)cc2)cc1]

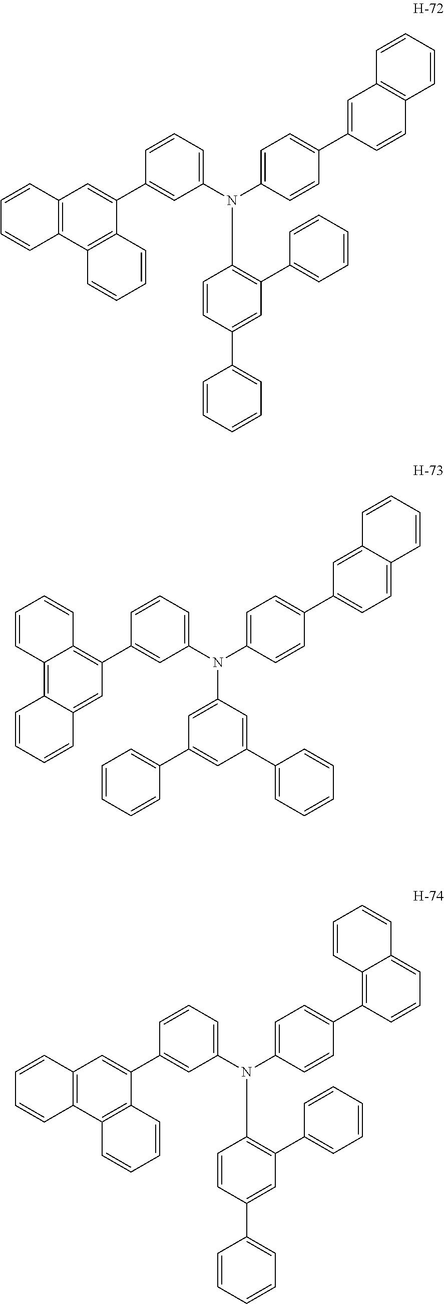 [SMILES: c1ccc(-c2ccc(N(c3ccc(-c4ccc5ccccc5c4)cc3)c3cccc(-c4cc5ccccc5c5ccccc45)c3)c(-c3ccccc3)c2)cc1 ; c1ccc(-c2cc(-c3ccccc3)cc(N(c3ccc(-c4ccc5ccccc5c4)cc3)c3cccc(-c4cc5ccccc5c5ccccc45)c3)c2)cc1 ; c1ccc(-c2ccc(N(c3ccc(-c4cccc5ccccc45)cc3)c3cccc(-c4cc5ccccc5c5ccccc45)c3)c(-c3ccccc3)c2)cc1]

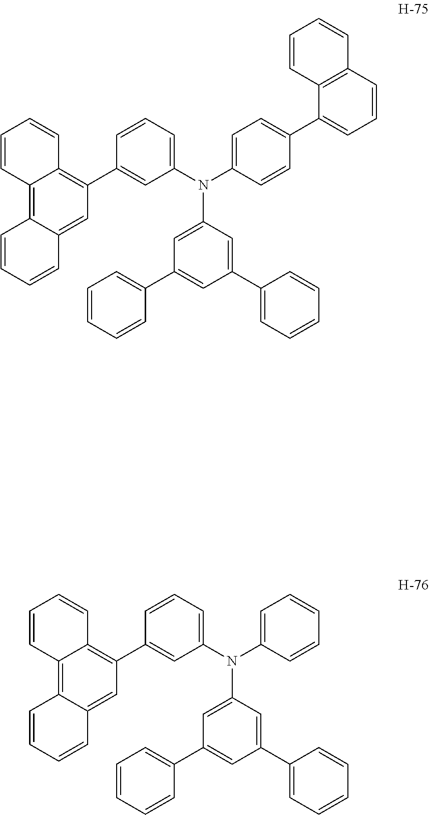 [SMILES: c1ccc(-c2cc(-c3ccccc3)cc(N(c3ccc(-c4cccc5ccccc45)cc3)c3cccc(-c4cc5ccccc5c5ccccc45)c3)c2)cc1 ; c1ccc(-c2cc(-c3ccccc3)cc(N(c3ccccc3)c3cccc(-c4cc5ccccc5c5ccccc45)c3)c2)cc1]

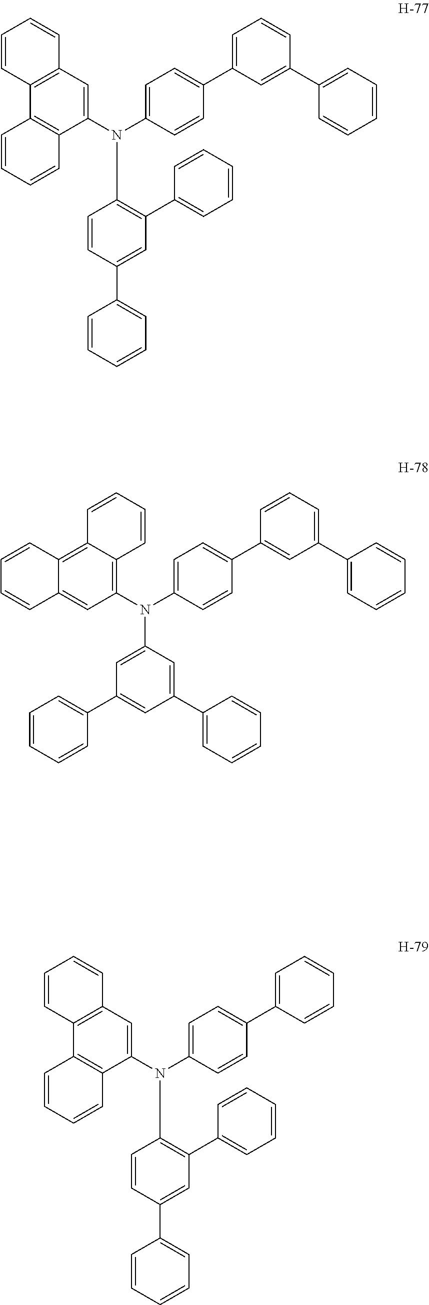 [SMILES: c1ccc(-c2cccc(-c3ccc(N(c4ccc(-c5ccccc5)cc4-c4ccccc4)c4cc5ccccc5c5ccccc45)cc3)c2)cc1 ; c1ccc(-c2cccc(-c3ccc(N(c4cc(-c5ccccc5)cc(-c5ccccc5)c4)c4cc5ccccc5c5ccccc45)cc3)c2)cc1 ; c1ccc(-c2ccc(N(c3ccc(-c4ccccc4)cc3-c3ccccc3)c3cc4ccccc4c4ccccc34)cc2)cc1]

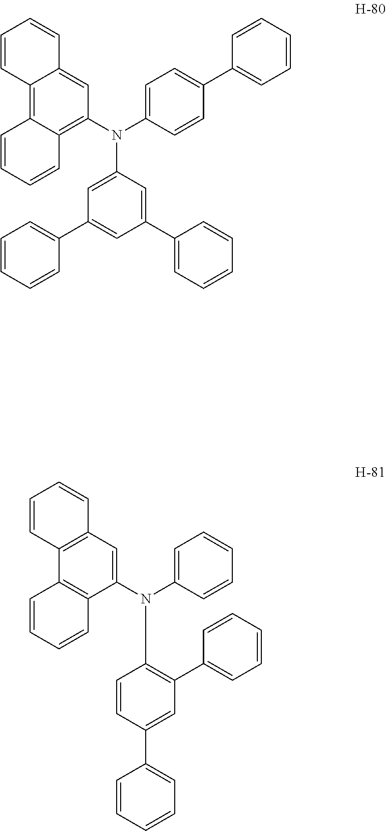 [SMILES: c1ccc(-c2ccc(N(c3cc(-c4ccccc4)cc(-c4ccccc4)c3)c3cc4ccccc4c4ccccc34)cc2)cc1 ; c1ccc(-c2ccc(N(c3ccccc3)c3cc4ccccc4c4ccccc34)c(-c3ccccc3)c2)cc1]

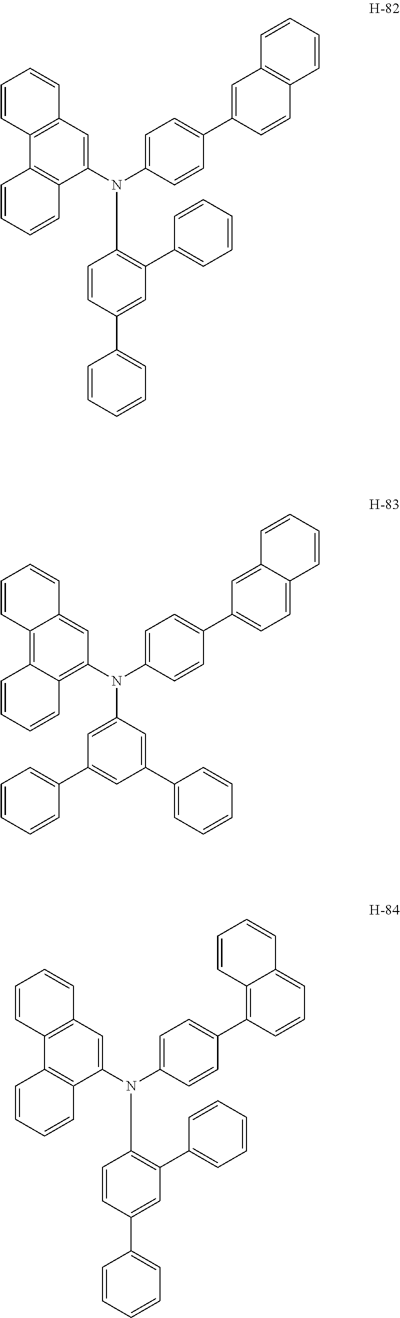 [SMILES: c1ccc(-c2ccc(N(c3ccc(-c4ccc5ccccc5c4)cc3)c3cc4ccccc4c4ccccc34)c(-c3ccccc3)c2)cc1 ; c1ccc(-c2cc(-c3ccccc3)cc(N(c3ccc(-c4ccc5ccccc5c4)cc3)c3cc4ccccc4c4ccccc34)c2)cc1 ; c1ccc(-c2ccc(N(c3ccc(-c4cccc5ccccc45)cc3)c3cc4ccccc4c4ccccc34)c(-c3ccccc3)c2)cc1]

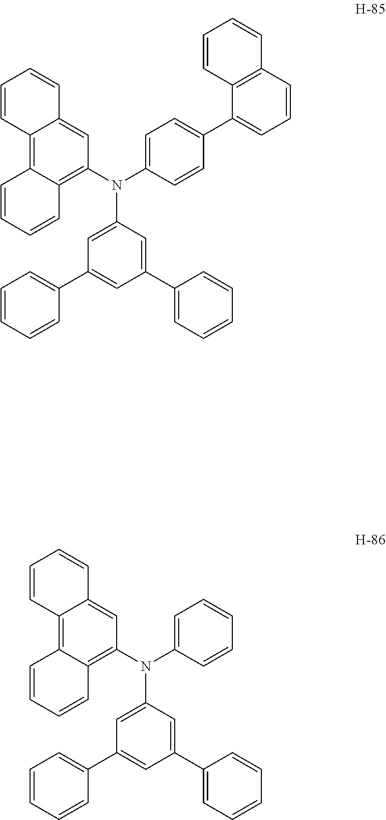 [SMILES: c1ccc(-c2cc(-c3ccccc3)cc(N(c3ccc(-c4cccc5ccccc45)cc3)c3cc4ccccc4c4ccccc34)c2)cc1 ; c1ccc(-c2cc(-c3ccccc3)cc(N(c3ccccc3)c3cc4ccccc4c4ccccc34)c2)cc1]

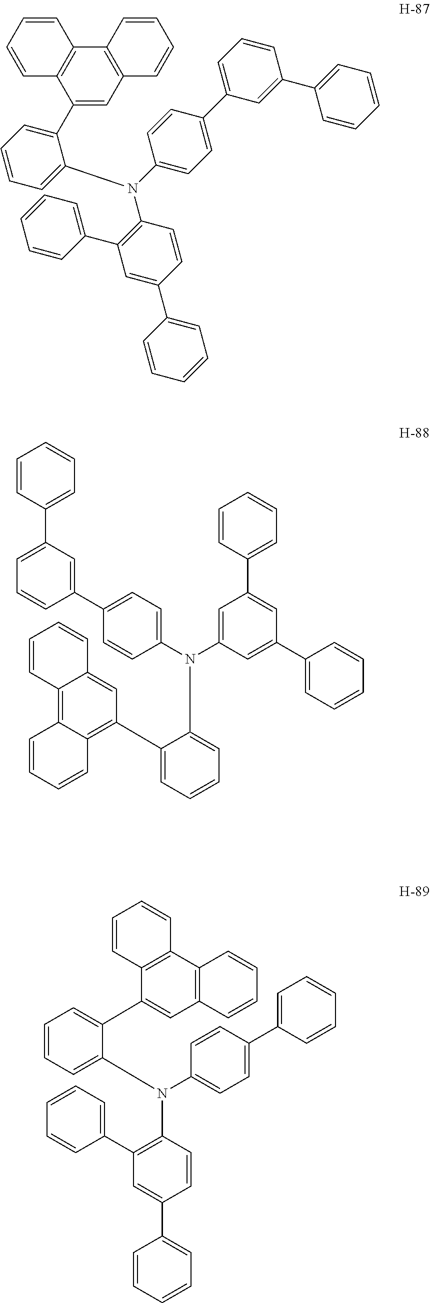 [SMILES: c1ccc(-c2cccc(-c3ccc(N(c4ccc(-c5ccccc5)cc4-c4ccccc4)c4ccccc4-c4cc5ccccc5c5ccccc45)cc3)c2)cc1 ; c1ccc(-c2cccc(-c3ccc(N(c4cc(-c5ccccc5)cc(-c5ccccc5)c4)c4ccccc4-c4cc5ccccc5c5ccccc45)cc3)c2)cc1 ; c1ccc(-c2ccc(N(c3ccc(-c4ccccc4)cc3-c3ccccc3)c3ccccc3-c3cc4ccccc4c4ccccc34)cc2)cc1]

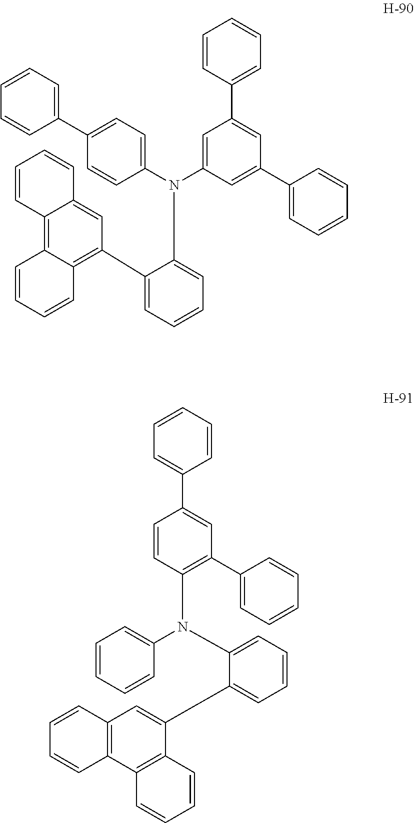 [SMILES: c1ccc(-c2ccc(N(c3cc(-c4ccccc4)cc(-c4ccccc4)c3)c3ccccc3-c3cc4ccccc4c4ccccc34)cc2)cc1 ; c1ccc(-c2ccc(N(c3ccccc3)c3ccccc3-c3cc4ccccc4c4ccccc34)c(-c3ccccc3)c2)cc1]

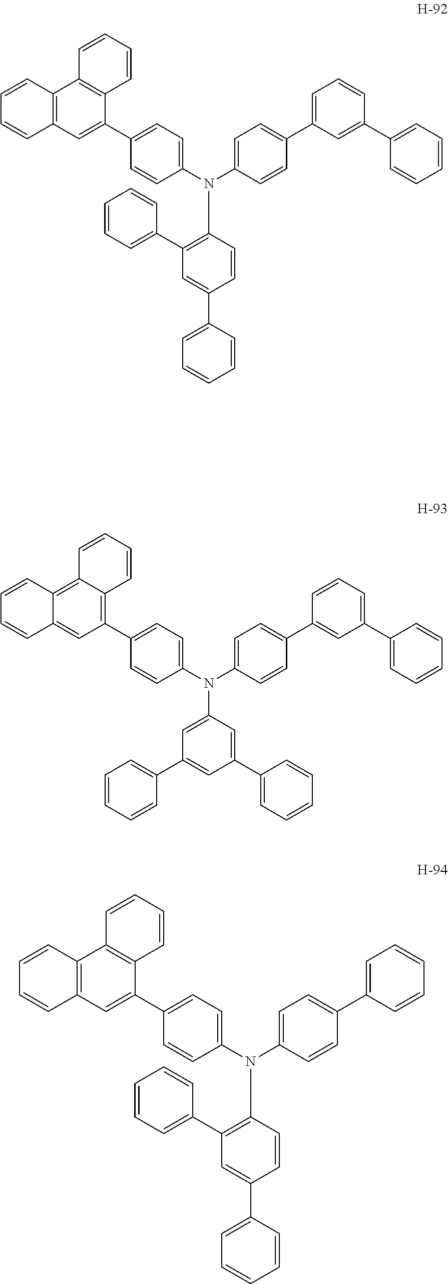 [SMILES: c1ccc(-c2cccc(-c3ccc(N(c4ccc(-c5cc6ccccc6c6ccccc56)cc4)c4ccc(-c5ccccc5)cc4-c4ccccc4)cc3)c2)cc1 ; c1ccc(-c2cccc(-c3ccc(N(c4ccc(-c5cc6ccccc6c6ccccc56)cc4)c4cc(-c5ccccc5)cc(-c5ccccc5)c4)cc3)c2)cc1 ; c1ccc(-c2ccc(N(c3ccc(-c4cc5ccccc5c5ccccc45)cc3)c3ccc(-c4ccccc4)cc3-c3ccccc3)cc2)cc1]

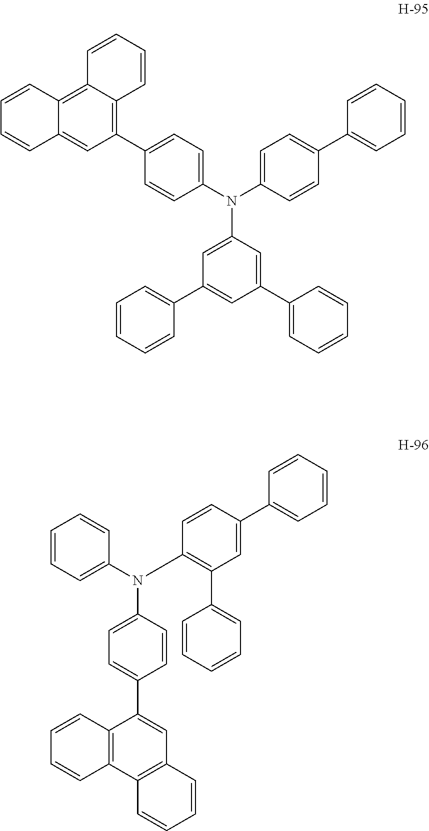 [SMILES: c1ccc(-c2ccc(N(c3ccc(-c4cc5ccccc5c5ccccc45)cc3)c3cc(-c4ccccc4)cc(-c4ccccc4)c3)cc2)cc1 ; c1ccc(-c2ccc(N(c3ccccc3)c3ccc(-c4cc5ccccc5c5ccccc45)cc3)c(-c3ccccc3)c2)cc1]

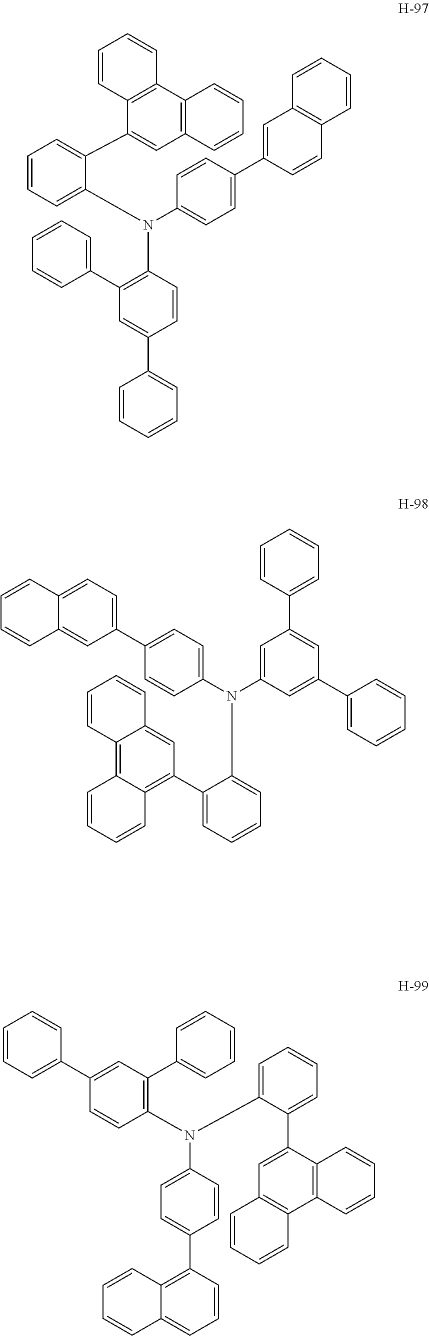 [SMILES: c1ccc(-c2ccc(N(c3ccc(-c4ccc5ccccc5c4)cc3)c3ccccc3-c3cc4ccccc4c4ccccc34)c(-c3ccccc3)c2)cc1 ; c1ccc(-c2cc(-c3ccccc3)cc(N(c3ccc(-c4ccc5ccccc5c4)cc3)c3ccccc3-c3cc4ccccc4c4ccccc34)c2)cc1 ; c1ccc(-c2ccc(N(c3ccc(-c4cccc5ccccc45)cc3)c3ccccc3-c3cc4ccccc4c4ccccc34)c(-c3ccccc3)c2)cc1]

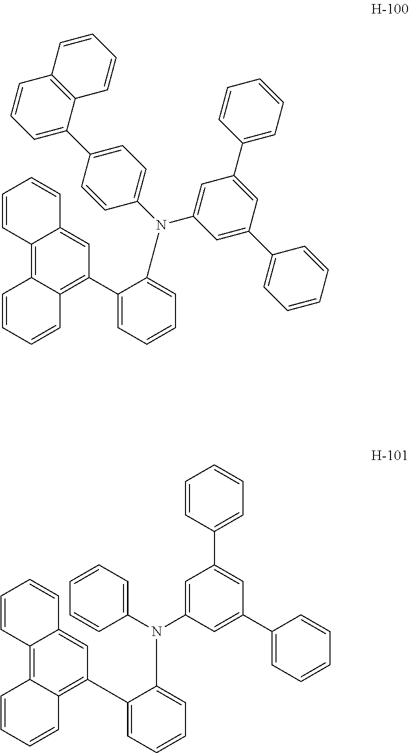 [SMILES: c1ccc(-c2cc(-c3ccccc3)cc(N(c3ccc(-c4cccc5ccccc45)cc3)c3ccccc3-c3cc4ccccc4c4ccccc34)c2)cc1 ; c1ccc(-c2cc(-c3ccccc3)cc(N(c3ccccc3)c3ccccc3-c3cc4ccccc4c4ccccc34)c2)cc1]

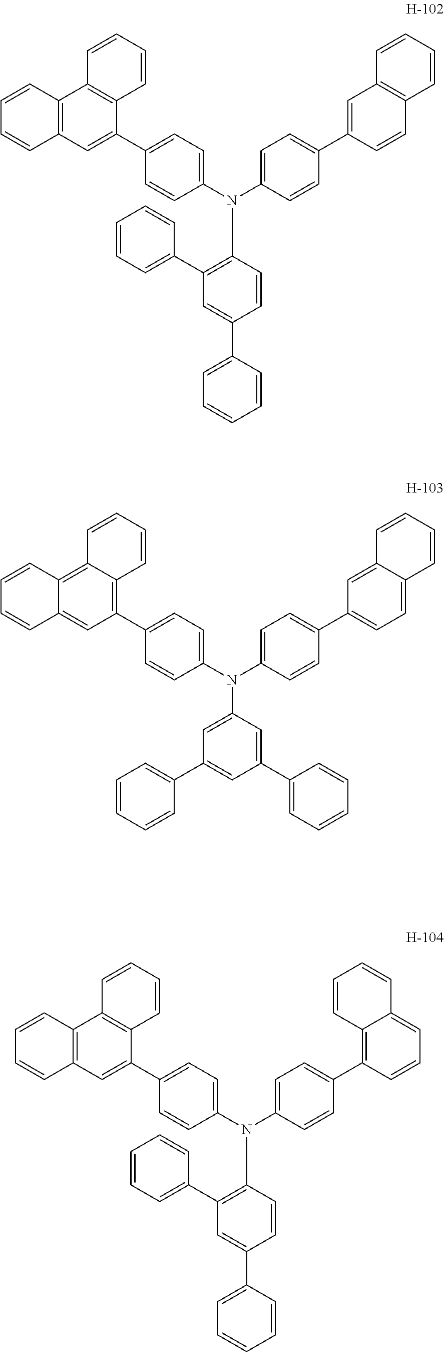 [SMILES: c1ccc(-c2ccc(N(c3ccc(-c4ccc5ccccc5c4)cc3)c3ccc(-c4cc5ccccc5c5ccccc45)cc3)c(-c3ccccc3)c2)cc1 ; c1ccc(-c2cc(-c3ccccc3)cc(N(c3ccc(-c4ccc5ccccc5c4)cc3)c3ccc(-c4cc5ccccc5c5ccccc45)cc3)c2)cc1 ; c1ccc(-c2ccc(N(c3ccc(-c4cccc5ccccc45)cc3)c3ccc(-c4cc5ccccc5c5ccccc45)cc3)c(-c3ccccc3)c2)cc1]

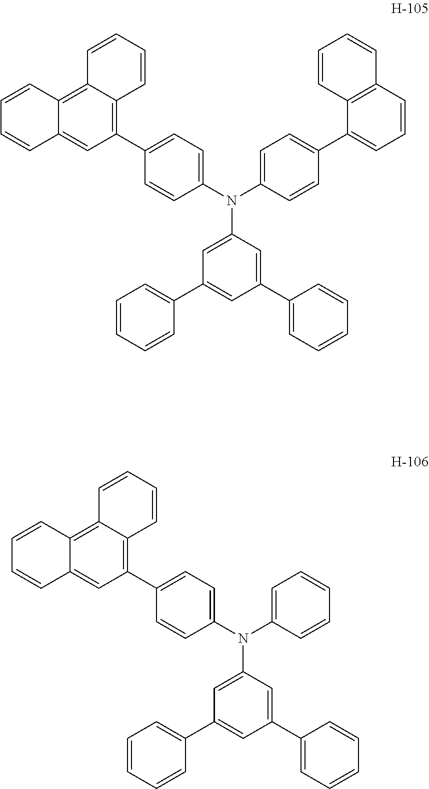 [SMILES: c1ccc(-c2cc(-c3ccccc3)cc(N(c3ccc(-c4cccc5ccccc45)cc3)c3ccc(-c4cc5ccccc5c5ccccc45)cc3)c2)cc1 ; c1ccc(-c2cc(-c3ccccc3)cc(N(c3ccccc3)c3ccc(-c4cc5ccccc5c5ccccc45)cc3)c2)cc1]

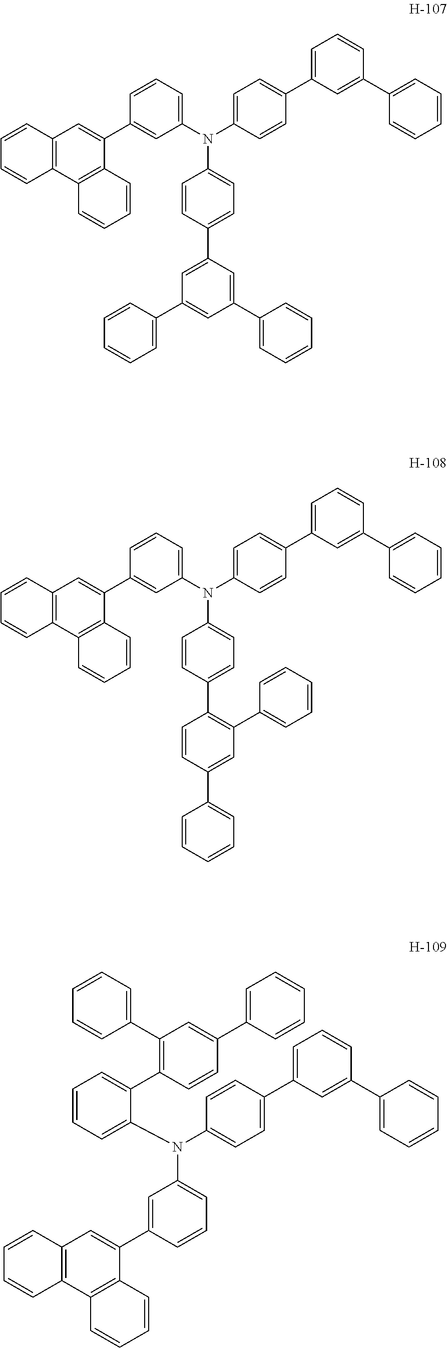 [SMILES: c1ccc(-c2cccc(-c3ccc(N(c4ccc(-c5cc(-c6ccccc6)cc(-c6ccccc6)c5)cc4)c4cccc(-c5cc6ccccc6c6ccccc56)c4)cc3)c2)cc1 ; c1ccc(-c2cccc(-c3ccc(N(c4ccc(-c5ccc(-c6ccccc6)cc5-c5ccccc5)cc4)c4cccc(-c5cc6ccccc6c6ccccc56)c4)cc3)c2)cc1 ; c1ccc(-c2cccc(-c3ccc(N(c4cccc(-c5cc6ccccc6c6ccccc56)c4)c4ccccc4-c4ccc(-c5ccccc5)cc4-c4ccccc4)cc3)c2)cc1]

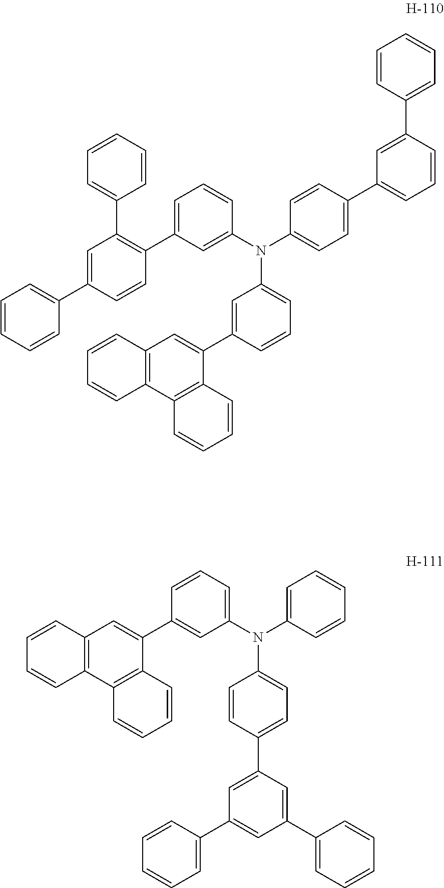 [SMILES: c1ccc(-c2cccc(-c3ccc(N(c4cccc(-c5ccc(-c6ccccc6)cc5-c5ccccc5)c4)c4cccc(-c5cc6ccccc6c6ccccc56)c4)cc3)c2)cc1 ; c1ccc(-c2cc(-c3ccccc3)cc(-c3ccc(N(c4ccccc4)c4cccc(-c5cc6ccccc6c6ccccc56)c4)cc3)c2)cc1]

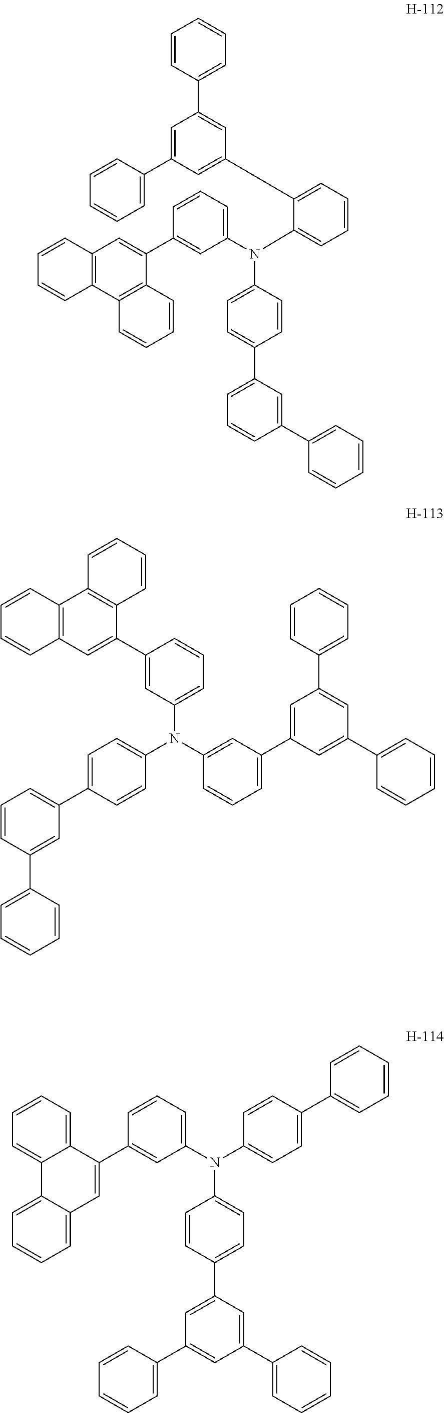 [SMILES: c1ccc(-c2cccc(-c3ccc(N(c4cccc(-c5cc6ccccc6c6ccccc56)c4)c4ccccc4-c4cc(-c5ccccc5)cc(-c5ccccc5)c4)cc3)c2)cc1 ; c1ccc(-c2cccc(-c3ccc(N(c4cccc(-c5cc(-c6ccccc6)cc(-c6ccccc6)c5)c4)c4cccc(-c5cc6ccccc6c6ccccc56)c4)cc3)c2)cc1 ; c1ccc(-c2ccc(N(c3ccc(-c4cc(-c5ccccc5)cc(-c5ccccc5)c4)cc3)c3cccc(-c4cc5ccccc5c5ccccc45)c3)cc2)cc1]

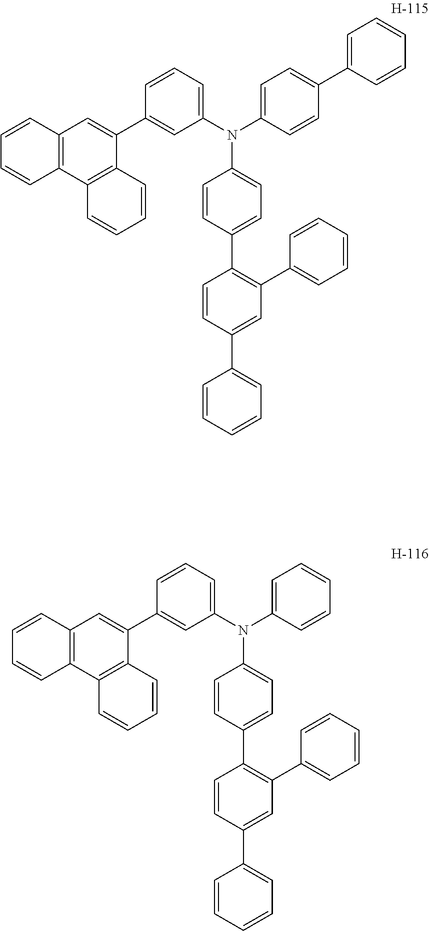 [SMILES: c1ccc(-c2ccc(N(c3ccc(-c4ccc(-c5ccccc5)cc4-c4ccccc4)cc3)c3cccc(-c4cc5ccccc5c5ccccc45)c3)cc2)cc1 ; c1ccc(-c2ccc(-c3ccc(N(c4ccccc4)c4cccc(-c5cc6ccccc6c6ccccc56)c4)cc3)c(-c3ccccc3)c2)cc1]

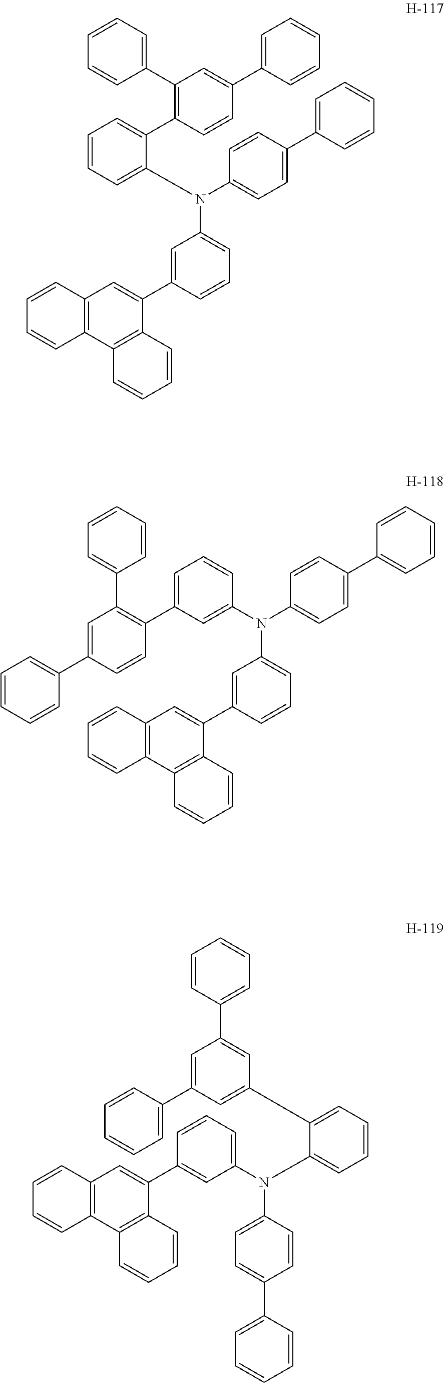 [SMILES: c1ccc(-c2ccc(N(c3cccc(-c4cc5ccccc5c5ccccc45)c3)c3ccccc3-c3ccc(-c4ccccc4)cc3-c3ccccc3)cc2)cc1 ; c1ccc(-c2ccc(N(c3cccc(-c4ccc(-c5ccccc5)cc4-c4ccccc4)c3)c3cccc(-c4cc5ccccc5c5ccccc45)c3)cc2)cc1 ; c1ccc(-c2ccc(N(c3cccc(-c4cc5ccccc5c5ccccc45)c3)c3ccccc3-c3cc(-c4ccccc4)cc(-c4ccccc4)c3)cc2)cc1]

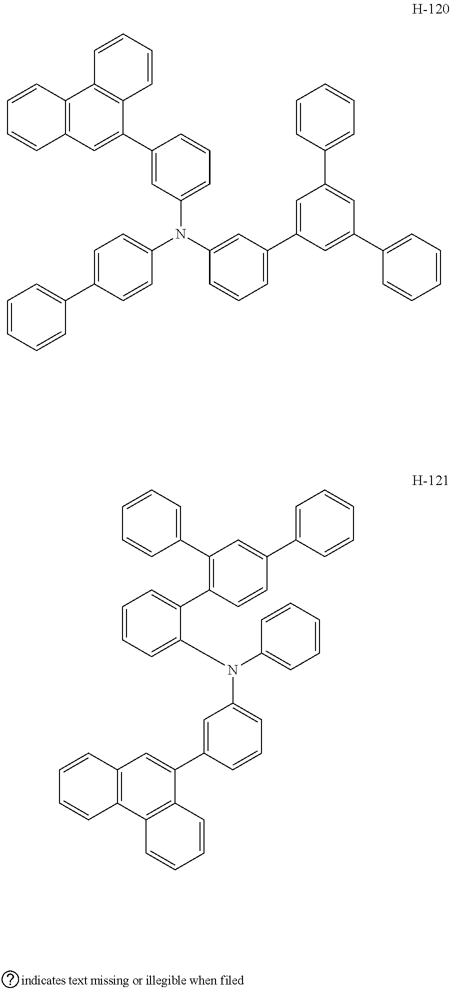 [SMILES: c1ccc(-c2ccc(N(c3cccc(-c4cc(-c5ccccc5)cc(-c5ccccc5)c4)c3)c3cccc(-c4cc5ccccc5c5ccccc45)c3)cc2)cc1 ; c1ccc(-c2ccc(-c3ccccc3N(c3ccccc3)c3cccc(-c4cc5ccccc5c5ccccc45)c3)c(-c3ccccc3)c2)cc1]

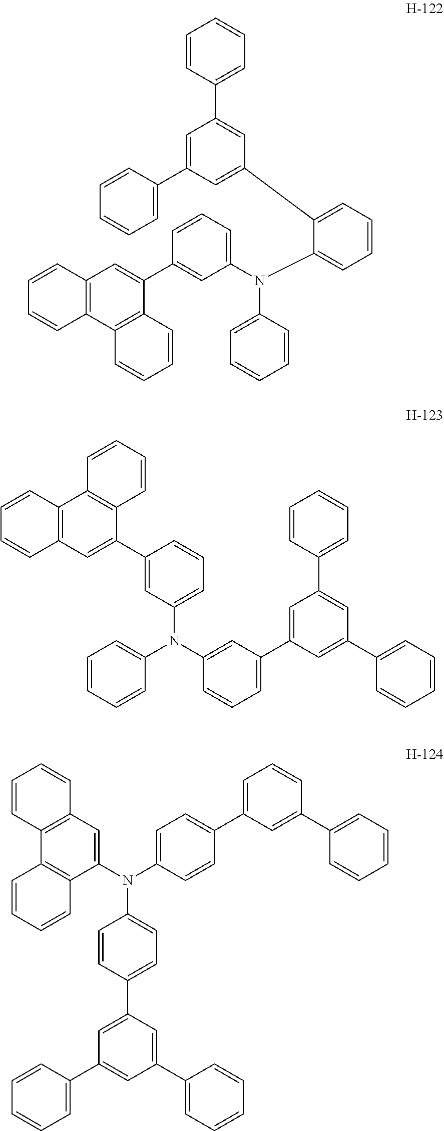 [SMILES: c1ccc(-c2cc(-c3ccccc3)cc(-c3ccccc3N(c3ccccc3)c3cccc(-c4cc5ccccc5c5ccccc45)c3)c2)cc1 ; c1ccc(-c2cc(-c3ccccc3)cc(-c3cccc(N(c4ccccc4)c4cccc(-c5cc6ccccc6c6ccccc56)c4)c3)c2)cc1 ; c1ccc(-c2cccc(-c3ccc(N(c4ccc(-c5cc(-c6ccccc6)cc(-c6ccccc6)c5)cc4)c4cc5ccccc5c5ccccc45)cc3)c2)cc1]

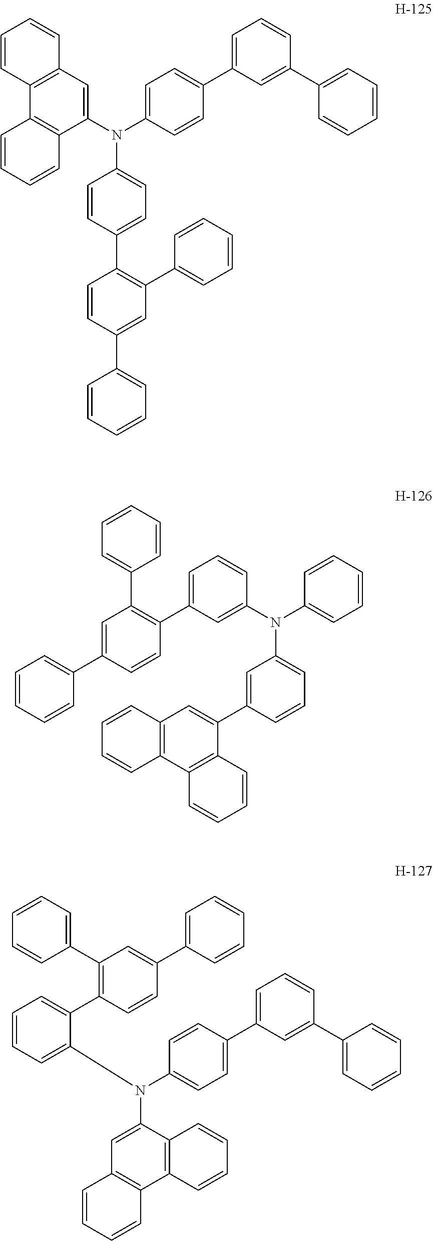 [SMILES: c1ccc(-c2cccc(-c3ccc(N(c4ccc(-c5ccc(-c6ccccc6)cc5-c5ccccc5)cc4)c4cc5ccccc5c5ccccc45)cc3)c2)cc1 ; c1ccc(-c2ccc(-c3cccc(N(c4ccccc4)c4cccc(-c5cc6ccccc6c6ccccc56)c4)c3)c(-c3ccccc3)c2)cc1 ; c1ccc(-c2cccc(-c3ccc(N(c4ccccc4-c4ccc(-c5ccccc5)cc4-c4ccccc4)c4cc5ccccc5c5ccccc45)cc3)c2)cc1]

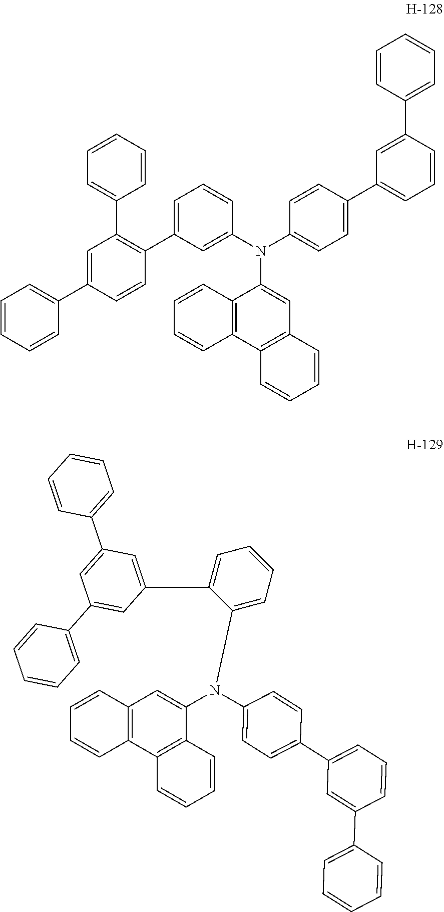 [SMILES: c1ccc(-c2cccc(-c3ccc(N(c4cccc(-c5ccc(-c6ccccc6)cc5-c5ccccc5)c4)c4cc5ccccc5c5ccccc45)cc3)c2)cc1 ; c1ccc(-c2cccc(-c3ccc(N(c4ccccc4-c4cc(-c5ccccc5)cc(-c5ccccc5)c4)c4cc5ccccc5c5ccccc45)cc3)c2)cc1]

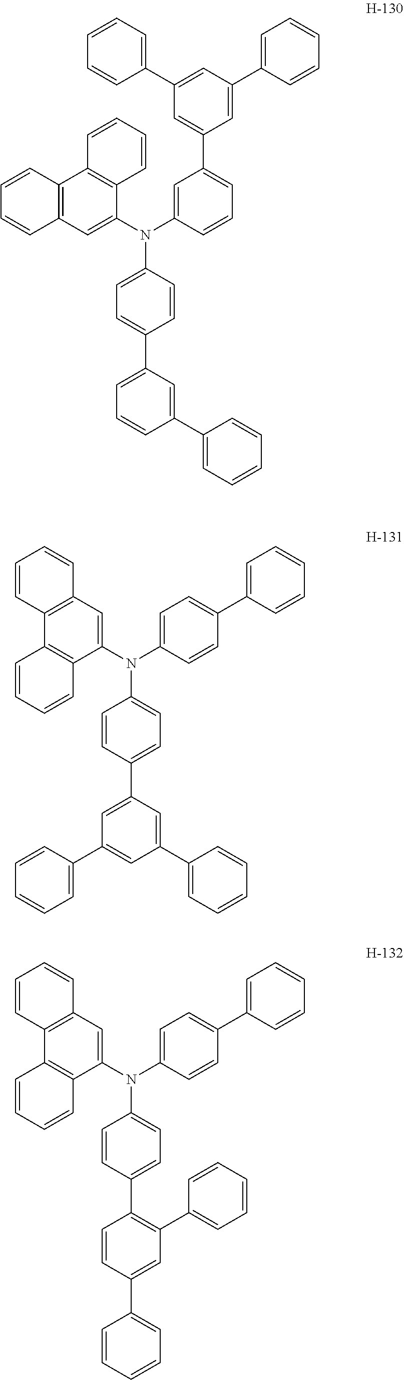 [SMILES: c1ccc(-c2cccc(-c3ccc(N(c4cccc(-c5cc(-c6ccccc6)cc(-c6ccccc6)c5)c4)c4cc5ccccc5c5ccccc45)cc3)c2)cc1 ; c1ccc(-c2ccc(N(c3ccc(-c4cc(-c5ccccc5)cc(-c5ccccc5)c4)cc3)c3cc4ccccc4c4ccccc34)cc2)cc1 ; c1ccc(-c2ccc(N(c3ccc(-c4ccc(-c5ccccc5)cc4-c4ccccc4)cc3)c3cc4ccccc4c4ccccc34)cc2)cc1]

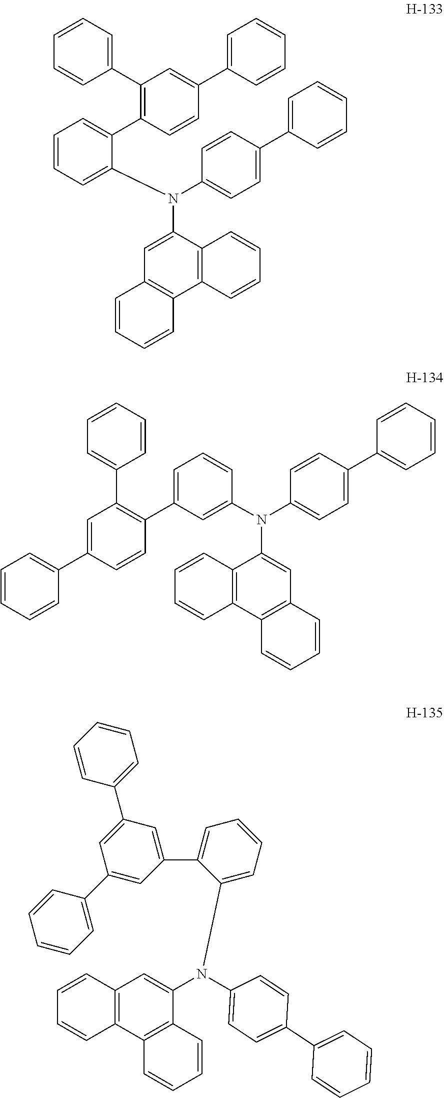 [SMILES: c1ccc(-c2ccc(N(c3ccccc3-c3ccc(-c4ccccc4)cc3-c3ccccc3)c3cc4ccccc4c4ccccc34)cc2)cc1 ; c1ccc(-c2ccc(N(c3cccc(-c4ccc(-c5ccccc5)cc4-c4ccccc4)c3)c3cc4ccccc4c4ccccc34)cc2)cc1 ; c1ccc(-c2ccc(N(c3ccccc3-c3cc(-c4ccccc4)cc(-c4ccccc4)c3)c3cc4ccccc4c4ccccc34)cc2)cc1]

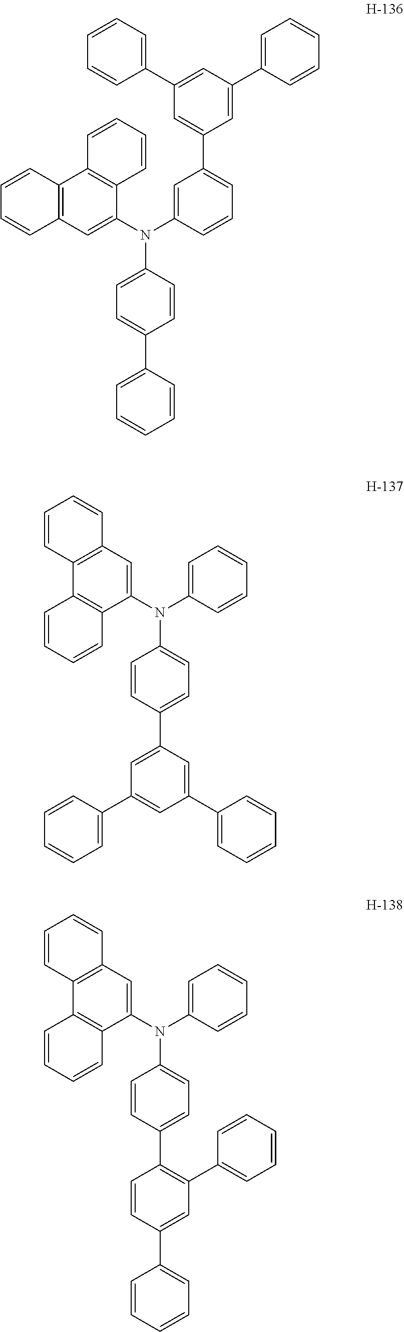 [SMILES: c1ccc(-c2ccc(N(c3cccc(-c4cc(-c5ccccc5)cc(-c5ccccc5)c4)c3)c3cc4ccccc4c4ccccc34)cc2)cc1 ; c1ccc(-c2cc(-c3ccccc3)cc(-c3ccc(N(c4ccccc4)c4cc5ccccc5c5ccccc45)cc3)c2)cc1 ; c1ccc(-c2ccc(-c3ccc(N(c4ccccc4)c4cc5ccccc5c5ccccc45)cc3)c(-c3ccccc3)c2)cc1]

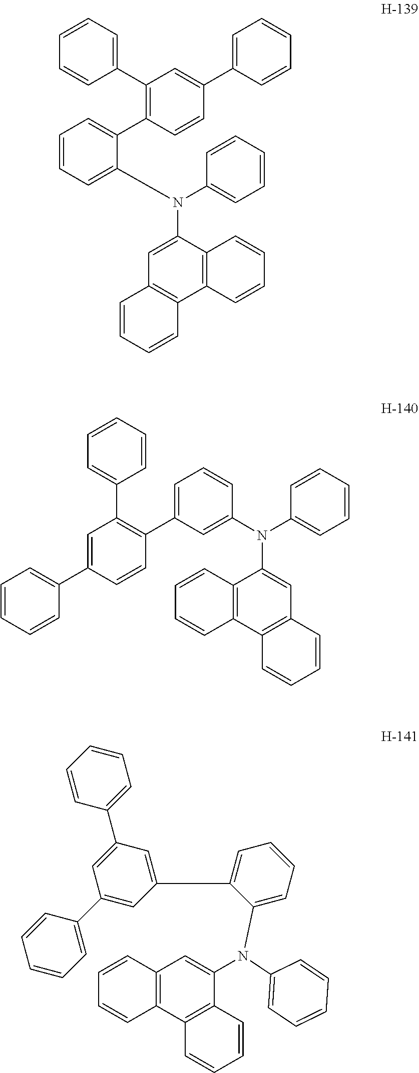 [SMILES: c1ccc(-c2ccc(-c3ccccc3N(c3ccccc3)c3cc4ccccc4c4ccccc34)c(-c3ccccc3)c2)cc1 ; c1ccc(-c2ccc(-c3cccc(N(c4ccccc4)c4cc5ccccc5c5ccccc45)c3)c(-c3ccccc3)c2)cc1 ; c1ccc(-c2cc(-c3ccccc3)cc(-c3ccccc3N(c3ccccc3)c3cc4ccccc4c4ccccc34)c2)cc1]

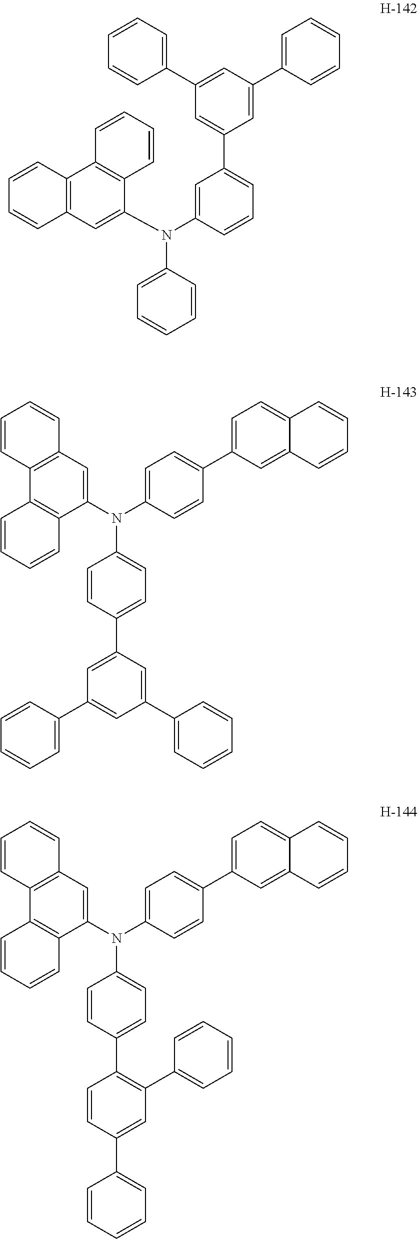 [SMILES: c1ccc(-c2cc(-c3ccccc3)cc(-c3cccc(N(c4ccccc4)c4cc5ccccc5c5ccccc45)c3)c2)cc1 ; c1ccc(-c2cc(-c3ccccc3)cc(-c3ccc(N(c4ccc(-c5ccc6ccccc6c5)cc4)c4cc5ccccc5c5ccccc45)cc3)c2)cc1 ; c1ccc(-c2ccc(-c3ccc(N(c4ccc(-c5ccc6ccccc6c5)cc4)c4cc5ccccc5c5ccccc45)cc3)c(-c3ccccc3)c2)cc1]

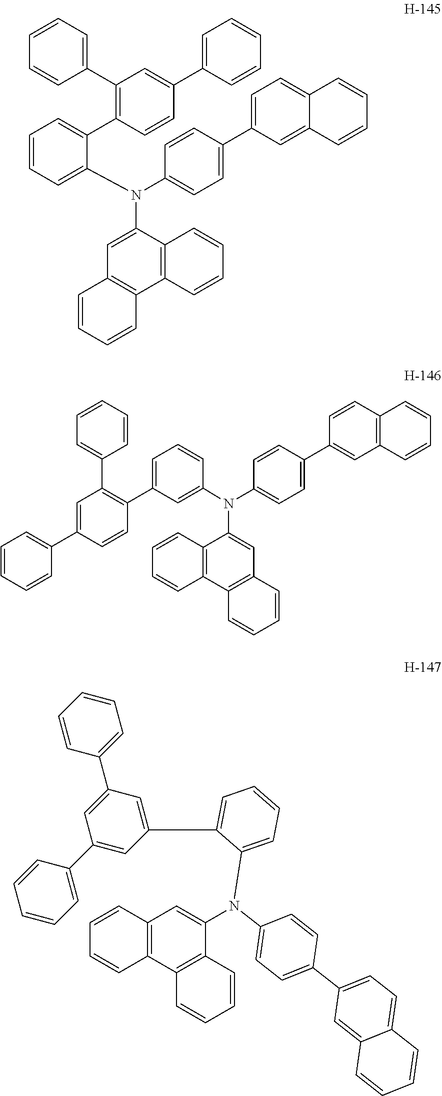 [SMILES: c1ccc(-c2ccc(-c3ccccc3N(c3ccc(-c4ccc5ccccc5c4)cc3)c3cc4ccccc4c4ccccc34)c(-c3ccccc3)c2)cc1 ; c1ccc(-c2ccc(-c3cccc(N(c4ccc(-c5ccc6ccccc6c5)cc4)c4cc5ccccc5c5ccccc45)c3)c(-c3ccccc3)c2)cc1 ; c1ccc(-c2cc(-c3ccccc3)cc(-c3ccccc3N(c3ccc(-c4ccc5ccccc5c4)cc3)c3cc4ccccc4c4ccccc34)c2)cc1]

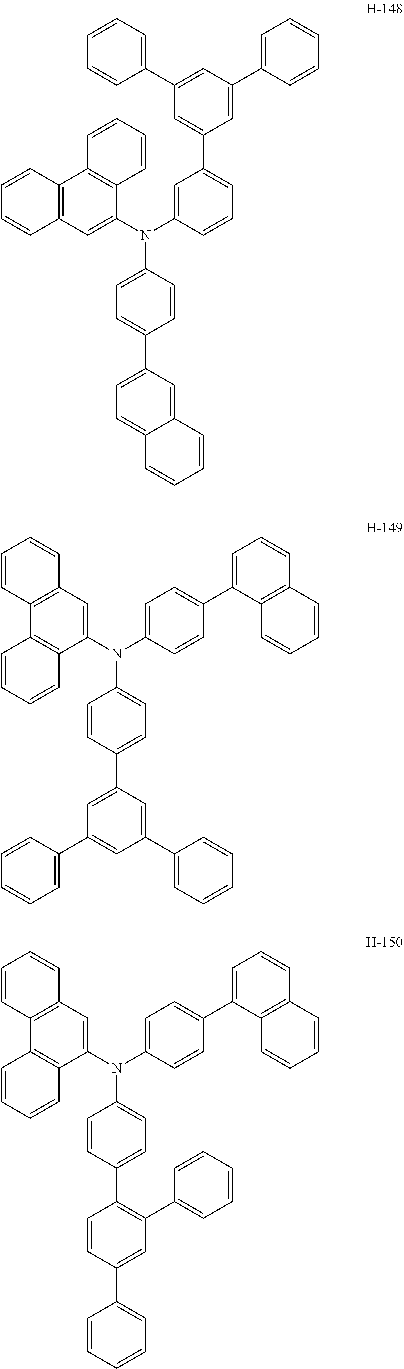 [SMILES: c1ccc(-c2cc(-c3ccccc3)cc(-c3cccc(N(c4ccc(-c5ccc6ccccc6c5)cc4)c4cc5ccccc5c5ccccc45)c3)c2)cc1 ; c1ccc(-c2cc(-c3ccccc3)cc(-c3ccc(N(c4ccc(-c5cccc6ccccc56)cc4)c4cc5ccccc5c5ccccc45)cc3)c2)cc1 ; c1ccc(-c2ccc(-c3ccc(N(c4ccc(-c5cccc6ccccc56)cc4)c4cc5ccccc5c5ccccc45)cc3)c(-c3ccccc3)c2)cc1]

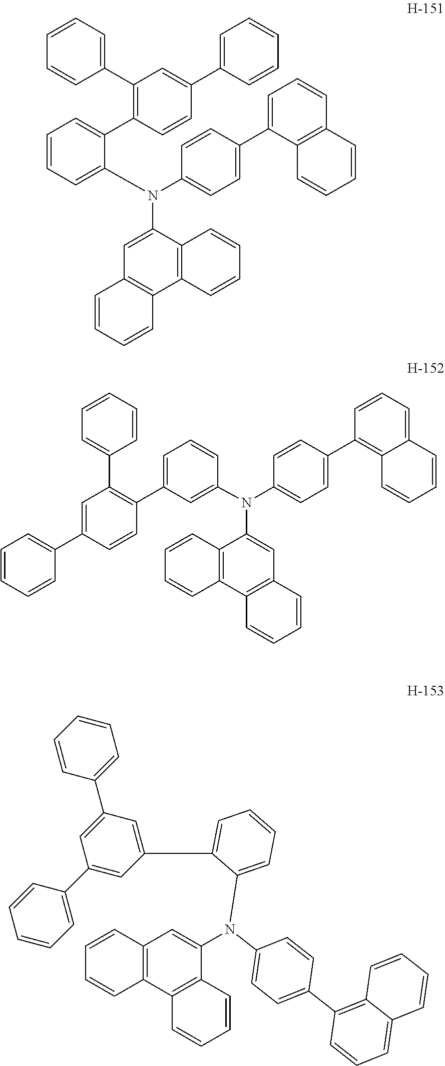 [SMILES: c1ccc(-c2ccc(-c3ccccc3N(c3ccc(-c4cccc5ccccc45)cc3)c3cc4ccccc4c4ccccc34)c(-c3ccccc3)c2)cc1 ; c1ccc(-c2ccc(-c3cccc(N(c4ccc(-c5cccc6ccccc56)cc4)c4cc5ccccc5c5ccccc45)c3)c(-c3ccccc3)c2)cc1 ; c1ccc(-c2cc(-c3ccccc3)cc(-c3ccccc3N(c3ccc(-c4cccc5ccccc45)cc3)c3cc4ccccc4c4ccccc34)c2)cc1]

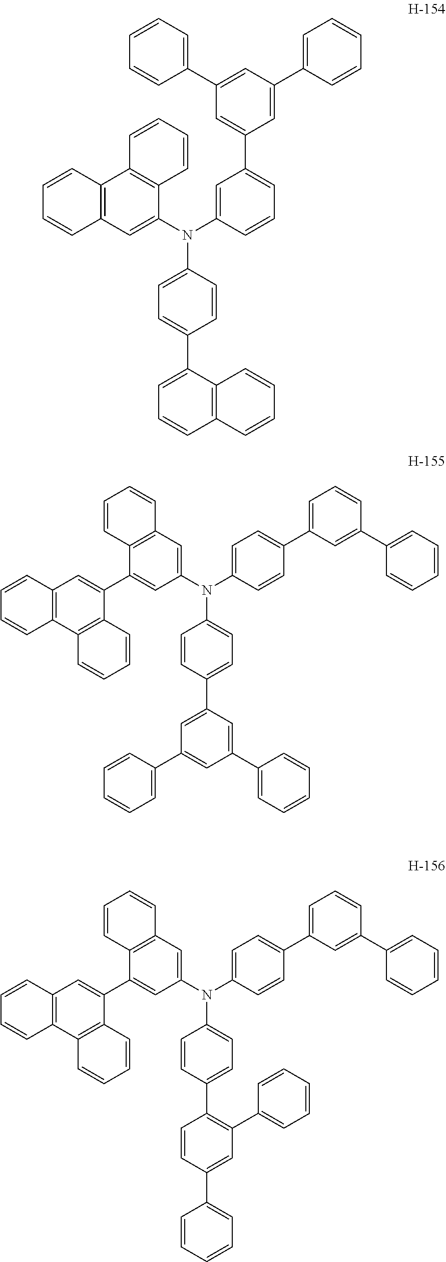 [SMILES: c1ccc(-c2cc(-c3ccccc3)cc(-c3cccc(N(c4ccc(-c5cccc6ccccc56)cc4)c4cc5ccccc5c5ccccc45)c3)c2)cc1 ; c1ccc(-c2cccc(-c3ccc(N(c4ccc(-c5cc(-c6ccccc6)cc(-c6ccccc6)c5)cc4)c4cc(-c5cc6ccccc6c6ccccc56)c5ccccc5c4)cc3)c2)cc1 ; c1ccc(-c2cccc(-c3ccc(N(c4ccc(-c5ccc(-c6ccccc6)cc5-c5ccccc5)cc4)c4cc(-c5cc6ccccc6c6ccccc56)c5ccccc5c4)cc3)c2)cc1]

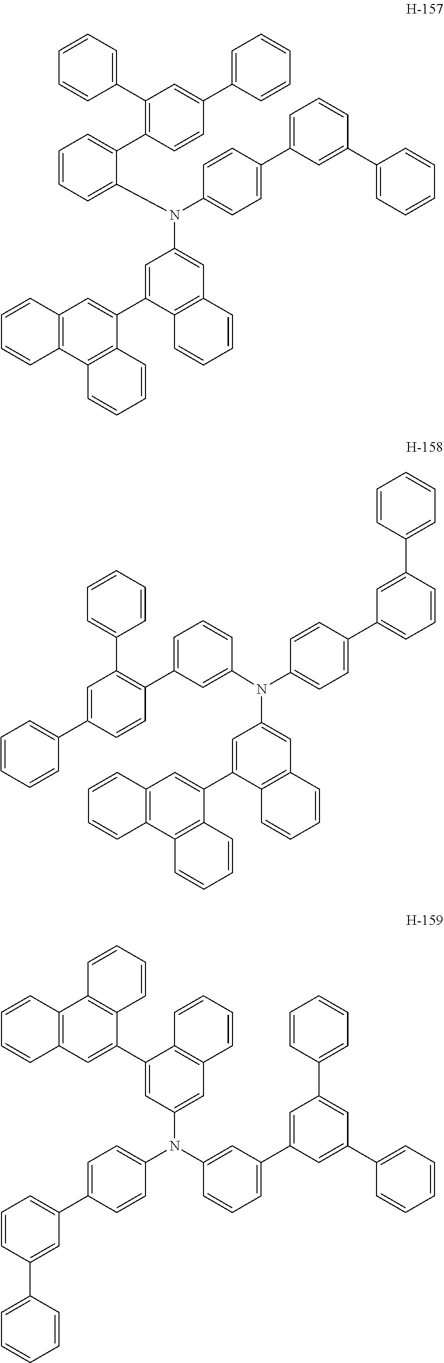 [SMILES: c1ccc(-c2cccc(-c3ccc(N(c4cc(-c5cc6ccccc6c6ccccc56)c5ccccc5c4)c4ccccc4-c4ccc(-c5ccccc5)cc4-c4ccccc4)cc3)c2)cc1 ; c1ccc(-c2cccc(-c3ccc(N(c4cccc(-c5ccc(-c6ccccc6)cc5-c5ccccc5)c4)c4cc(-c5cc6ccccc6c6ccccc56)c5ccccc5c4)cc3)c2)cc1 ; c1ccc(-c2cccc(-c3ccc(N(c4cccc(-c5cc(-c6ccccc6)cc(-c6ccccc6)c5)c4)c4cc(-c5cc6ccccc6c6ccccc56)c5ccccc5c4)cc3)c2)cc1]

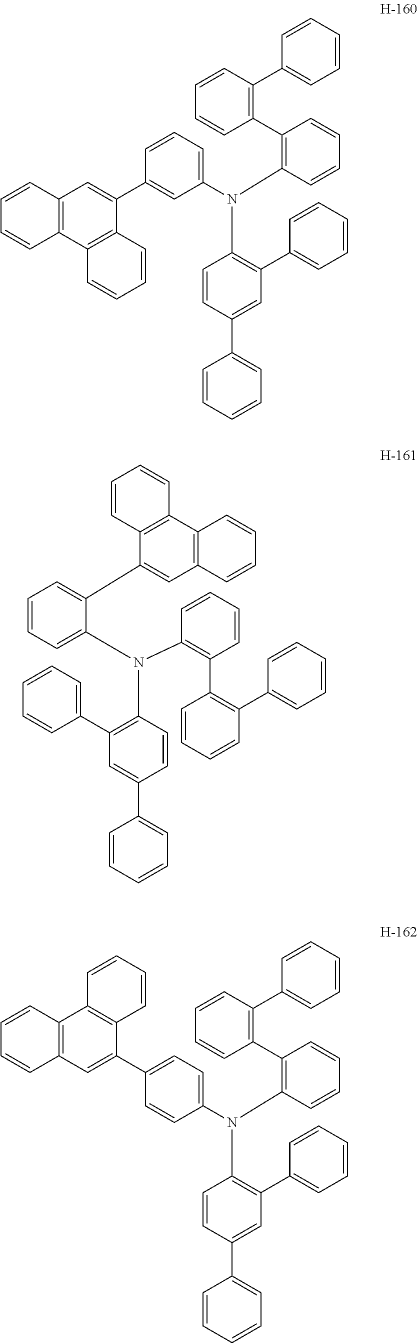 [SMILES: c1ccc(-c2ccc(N(c3cccc(-c4cc5ccccc5c5ccccc45)c3)c3ccccc3-c3ccccc3-c3ccccc3)c(-c3ccccc3)c2)cc1 ; c1ccc(-c2ccc(N(c3ccccc3-c3ccccc3-c3ccccc3)c3ccccc3-c3cc4ccccc4c4ccccc34)c(-c3ccccc3)c2)cc1 ; c1ccc(-c2ccc(N(c3ccc(-c4cc5ccccc5c5ccccc45)cc3)c3ccccc3-c3ccccc3-c3ccccc3)c(-c3ccccc3)c2)cc1]

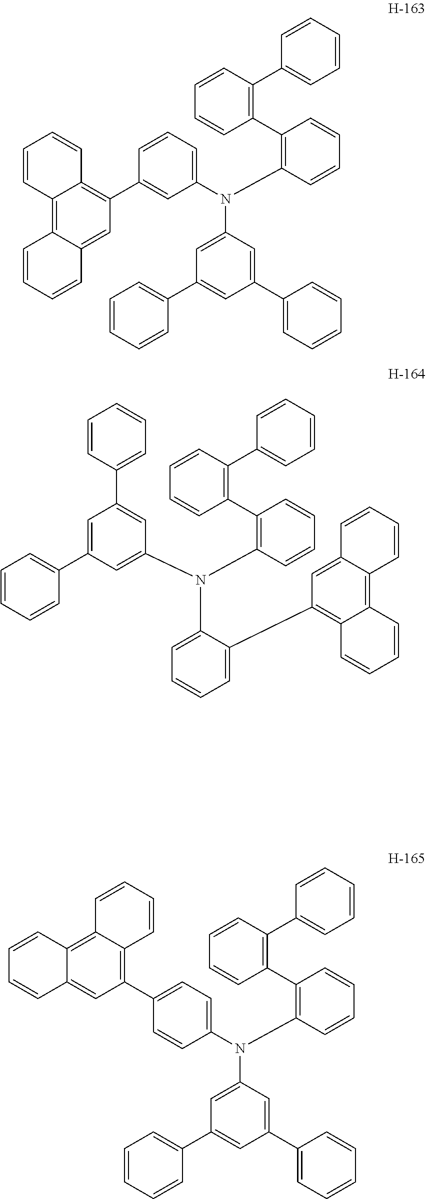 [SMILES: c1ccc(-c2cc(-c3ccccc3)cc(N(c3cccc(-c4cc5ccccc5c5ccccc45)c3)c3ccccc3-c3ccccc3-c3ccccc3)c2)cc1 ; c1ccc(-c2cc(-c3ccccc3)cc(N(c3ccccc3-c3ccccc3-c3ccccc3)c3ccccc3-c3cc4ccccc4c4ccccc34)c2)cc1 ; c1ccc(-c2cc(-c3ccccc3)cc(N(c3ccc(-c4cc5ccccc5c5ccccc45)cc3)c3ccccc3-c3ccccc3-c3ccccc3)c2)cc1]

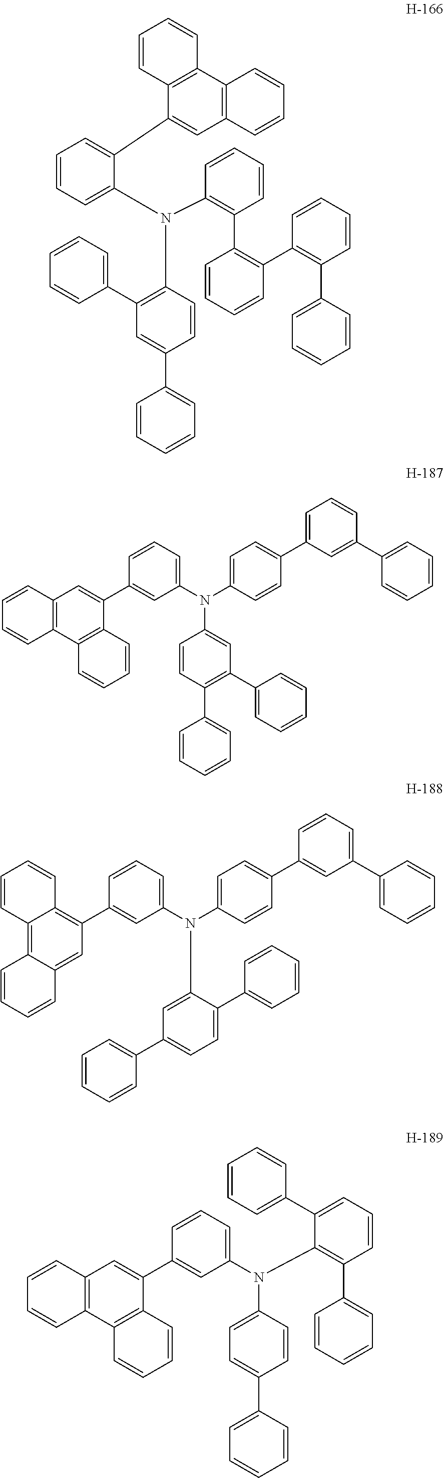 [SMILES: c1ccc(-c2ccc(N(c3ccccc3-c3ccccc3-c3ccccc3-c3ccccc3)c3ccccc3-c3cc4ccccc4c4ccccc34)c(-c3ccccc3)c2)cc1 ; c1ccc(-c2cccc(-c3ccc(N(c4cccc(-c5cc6ccccc6c6ccccc56)c4)c4ccc(-c5ccccc5)c(-c5ccccc5)c4)cc3)c2)cc1 ; c1ccc(-c2cccc(-c3ccc(N(c4cccc(-c5cc6ccccc6c6ccccc56)c4)c4cc(-c5ccccc5)ccc4-c4ccccc4)cc3)c2)cc1 ; c1ccc(-c2ccc(N(c3cccc(-c4cc5ccccc5c5ccccc45)c3)c3c(-c4ccccc4)cccc3-c3ccccc3)cc2)cc1]

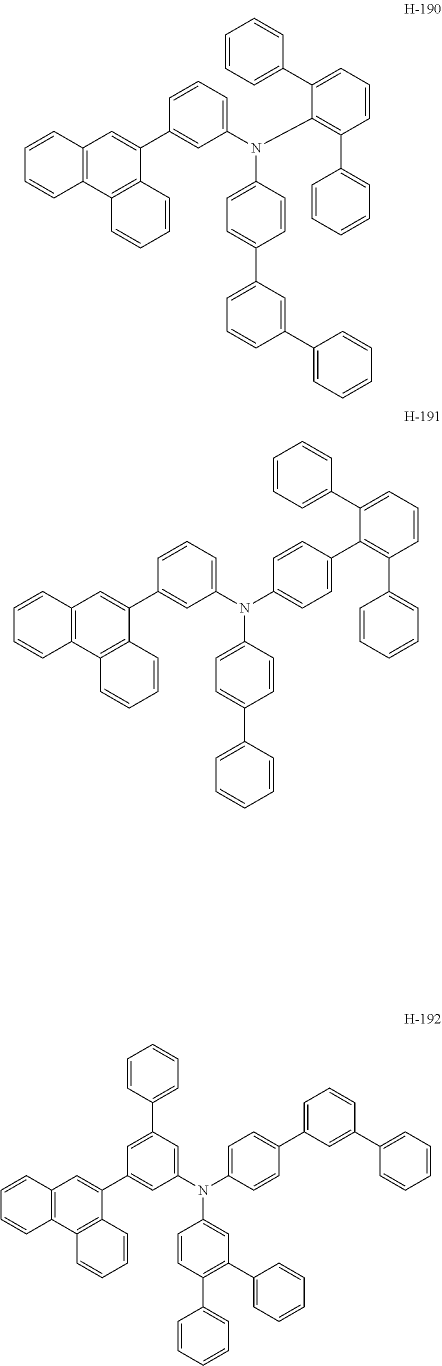 [SMILES: c1ccc(-c2cccc(-c3ccc(N(c4cccc(-c5cc6ccccc6c6ccccc56)c4)c4c(-c5ccccc5)cccc4-c4ccccc4)cc3)c2)cc1 ; c1ccc(-c2ccc(N(c3ccc(-c4c(-c5ccccc5)cccc4-c4ccccc4)cc3)c3cccc(-c4cc5ccccc5c5ccccc45)c3)cc2)cc1 ; c1ccc(-c2cccc(-c3ccc(N(c4cc(-c5ccccc5)cc(-c5cc6ccccc6c6ccccc56)c4)c4ccc(-c5ccccc5)c(-c5ccccc5)c4)cc3)c2)cc1]

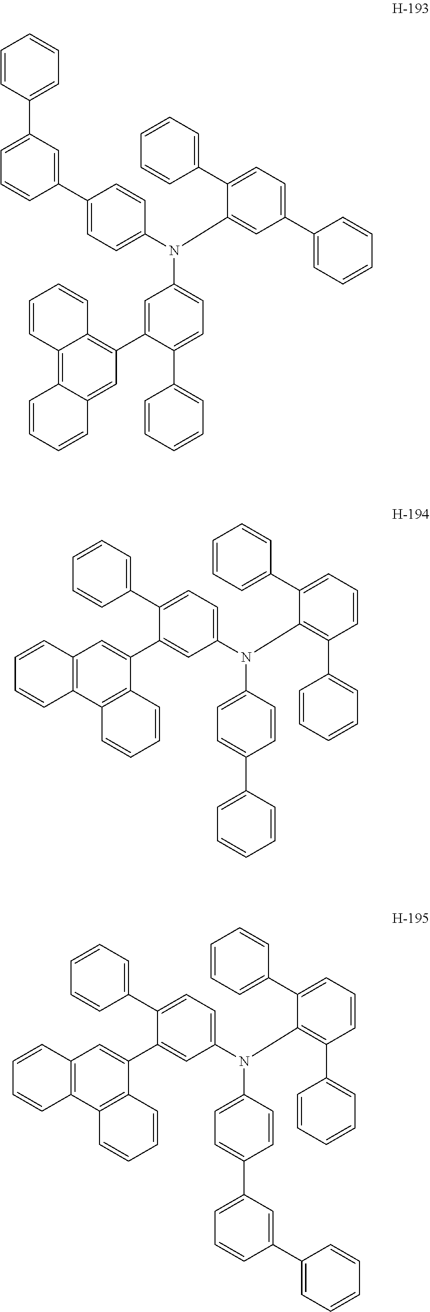 [SMILES: c1ccc(-c2cccc(-c3ccc(N(c4ccc(-c5ccccc5)c(-c5cc6ccccc6c6ccccc56)c4)c4cc(-c5ccccc5)ccc4-c4ccccc4)cc3)c2)cc1 ; c1ccc(-c2ccc(N(c3ccc(-c4ccccc4)c(-c4cc5ccccc5c5ccccc45)c3)c3c(-c4ccccc4)cccc3-c3ccccc3)cc2)cc1 ; c1ccc(-c2cccc(-c3ccc(N(c4ccc(-c5ccccc5)c(-c5cc6ccccc6c6ccccc56)c4)c4c(-c5ccccc5)cccc4-c4ccccc4)cc3)c2)cc1]

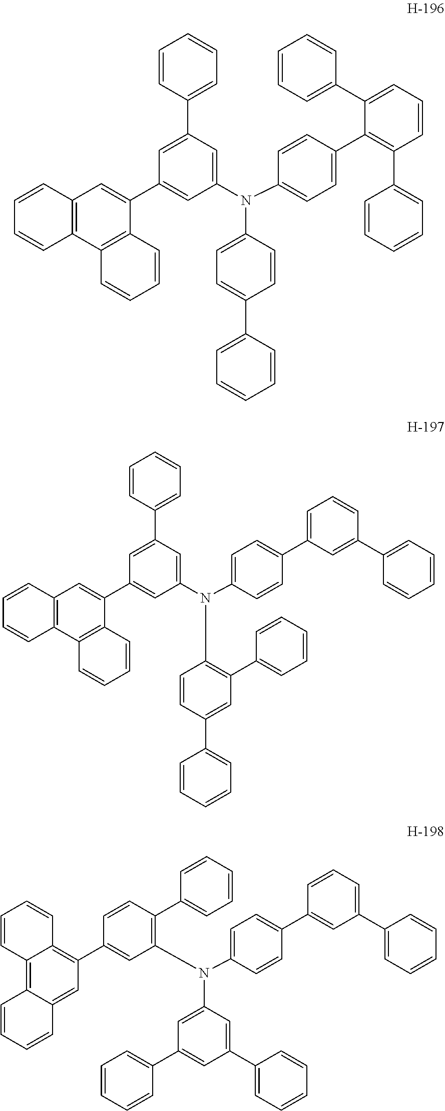 [SMILES: c1ccc(-c2ccc(N(c3ccc(-c4c(-c5ccccc5)cccc4-c4ccccc4)cc3)c3cc(-c4ccccc4)cc(-c4cc5ccccc5c5ccccc45)c3)cc2)cc1 ; c1ccc(-c2cccc(-c3ccc(N(c4cc(-c5ccccc5)cc(-c5cc6ccccc6c6ccccc56)c4)c4ccc(-c5ccccc5)cc4-c4ccccc4)cc3)c2)cc1 ; c1ccc(-c2cccc(-c3ccc(N(c4cc(-c5ccccc5)cc(-c5ccccc5)c4)c4cc(-c5cc6ccccc6c6ccccc56)ccc4-c4ccccc4)cc3)c2)cc1]

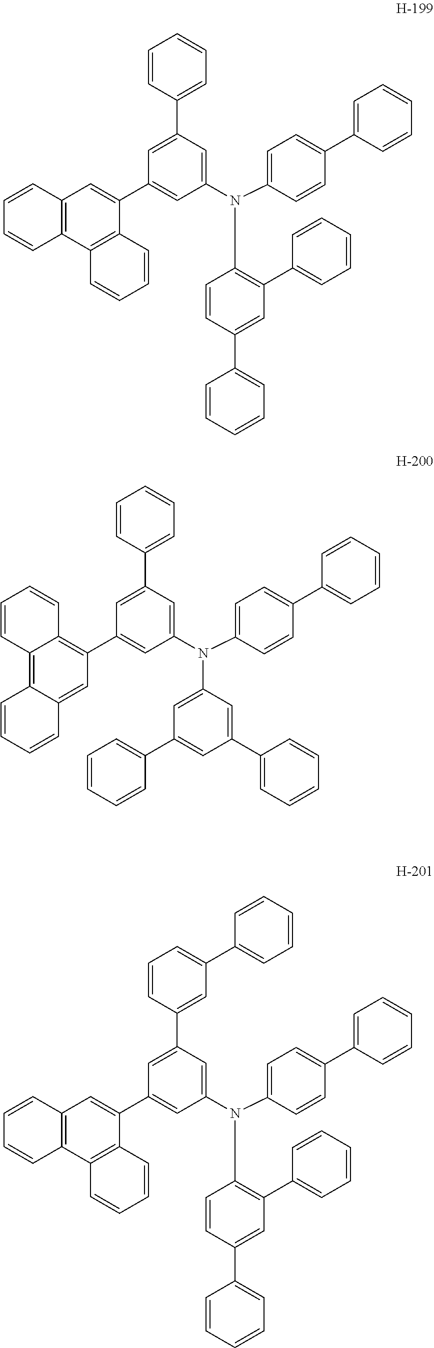 [SMILES: c1ccc(-c2ccc(N(c3cc(-c4ccccc4)cc(-c4cc5ccccc5c5ccccc45)c3)c3ccc(-c4ccccc4)cc3-c3ccccc3)cc2)cc1 ; c1ccc(-c2ccc(N(c3cc(-c4ccccc4)cc(-c4ccccc4)c3)c3cc(-c4ccccc4)cc(-c4cc5ccccc5c5ccccc45)c3)cc2)cc1 ; c1ccc(-c2ccc(N(c3cc(-c4cccc(-c5ccccc5)c4)cc(-c4cc5ccccc5c5ccccc45)c3)c3ccc(-c4ccccc4)cc3-c3ccccc3)cc2)cc1]

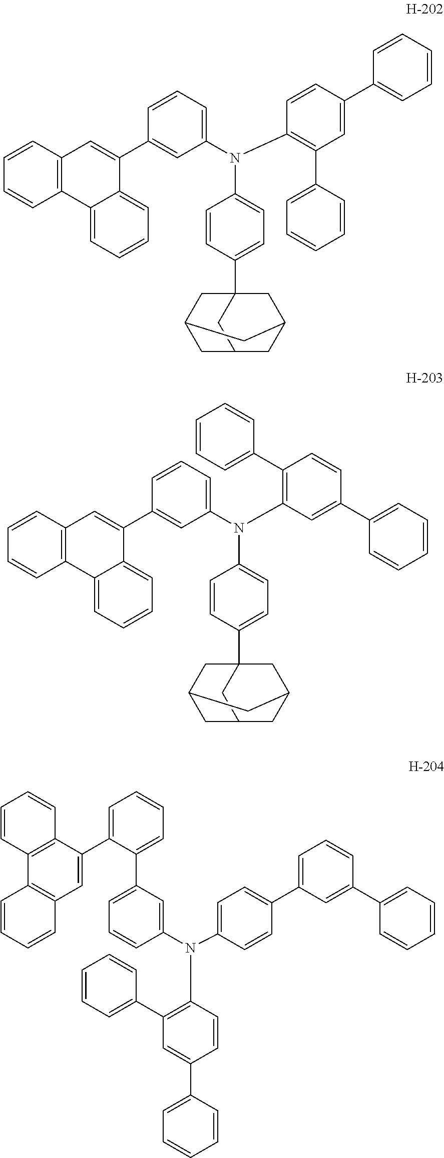 [SMILES: c1ccc(-c2ccc(N(c3ccc(C45CC6CC(CC(C6)C4)C5)cc3)c3cccc(-c4cc5ccccc5c5ccccc45)c3)c(-c3ccccc3)c2)cc1 ; c1ccc(-c2ccc(-c3ccccc3)c(N(c3ccc(C45CC6CC(CC(C6)C4)C5)cc3)c3cccc(-c4cc5ccccc5c5ccccc45)c3)c2)cc1 ; c1ccc(-c2cccc(-c3ccc(N(c4cccc(-c5ccccc5-c5cc6ccccc6c6ccccc56)c4)c4ccc(-c5ccccc5)cc4-c4ccccc4)cc3)c2)cc1]

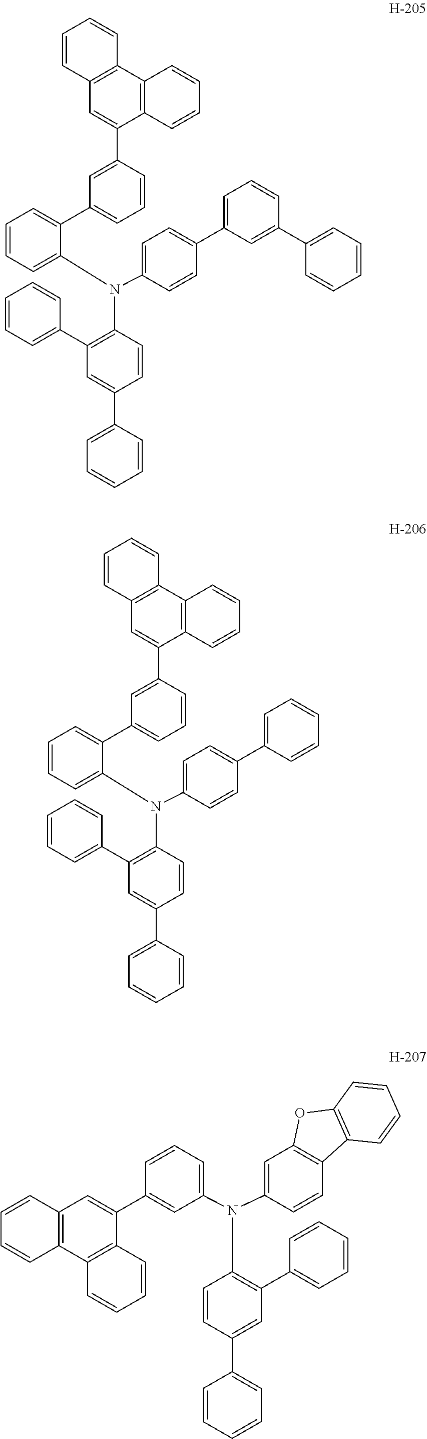 [SMILES: c1ccc(-c2cccc(-c3ccc(N(c4ccccc4-c4cccc(-c5cc6ccccc6c6ccccc56)c4)c4ccc(-c5ccccc5)cc4-c4ccccc4)cc3)c2)cc1 ; c1ccc(-c2ccc(N(c3ccccc3-c3cccc(-c4cc5ccccc5c5ccccc45)c3)c3ccc(-c4ccccc4)cc3-c3ccccc3)cc2)cc1 ; c1ccc(-c2ccc(N(c3cccc(-c4cc5ccccc5c5ccccc45)c3)c3ccc4c(c3)oc3ccccc34)c(-c3ccccc3)c2)cc1]

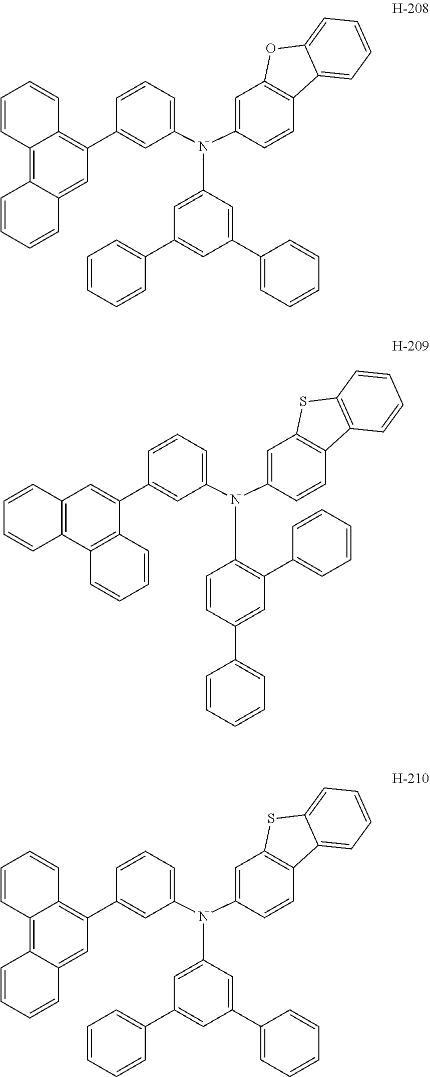 [SMILES: c1ccc(-c2cc(-c3ccccc3)cc(N(c3cccc(-c4cc5ccccc5c5ccccc45)c3)c3ccc4c(c3)oc3ccccc34)c2)cc1 ; c1ccc(-c2ccc(N(c3cccc(-c4cc5ccccc5c5ccccc45)c3)c3ccc4c(c3)sc3ccccc34)c(-c3ccccc3)c2)cc1 ; c1ccc(-c2cc(-c3ccccc3)cc(N(c3cccc(-c4cc5ccccc5c5ccccc45)c3)c3ccc4c(c3)sc3ccccc34)c2)cc1]

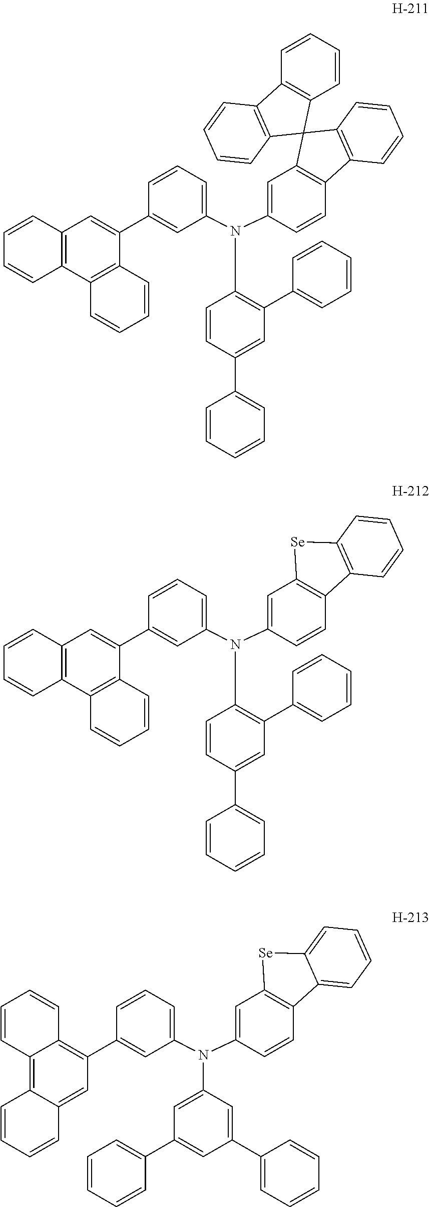 [SMILES: c1ccc(-c2ccc(N(c3cccc(-c4cc5ccccc5c5ccccc45)c3)c3ccc4c(c3)C3(c5ccccc5-c5ccccc53)c3ccccc3-4)c(-c3ccccc3)c2)cc1 ; c1ccc(-c2ccc(N(c3cccc(-c4cc5ccccc5c5ccccc45)c3)c3ccc4c(c3)[se]c3ccccc34)c(-c3ccccc3)c2)cc1 ; c1ccc(-c2cc(-c3ccccc3)cc(N(c3cccc(-c4cc5ccccc5c5ccccc45)c3)c3ccc4c(c3)[se]c3ccccc34)c2)cc1]

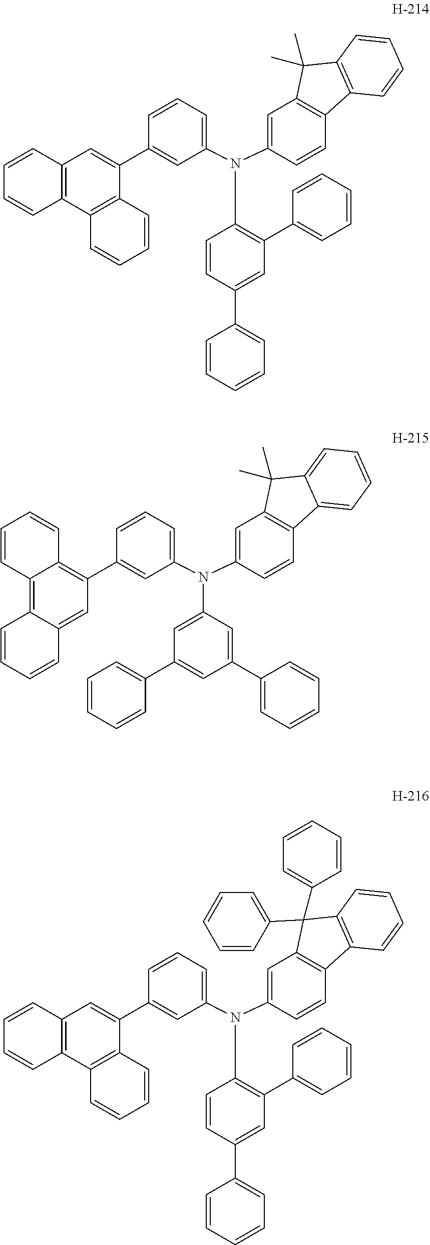 [SMILES: CC1(C)c2ccccc2-c2ccc(N(c3cccc(-c4cc5ccccc5c5ccccc45)c3)c3ccc(-c4ccccc4)cc3-c3ccccc3)cc21 ; CC1(C)c2ccccc2-c2ccc(N(c3cc(-c4ccccc4)cc(-c4ccccc4)c3)c3cccc(-c4cc5ccccc5c5ccccc45)c3)cc21 ; c1ccc(-c2ccc(N(c3cccc(-c4cc5ccccc5c5ccccc45)c3)c3ccc4c(c3)C(c3ccccc3)(c3ccccc3)c3ccccc3-4)c(-c3ccccc3)c2)cc1]

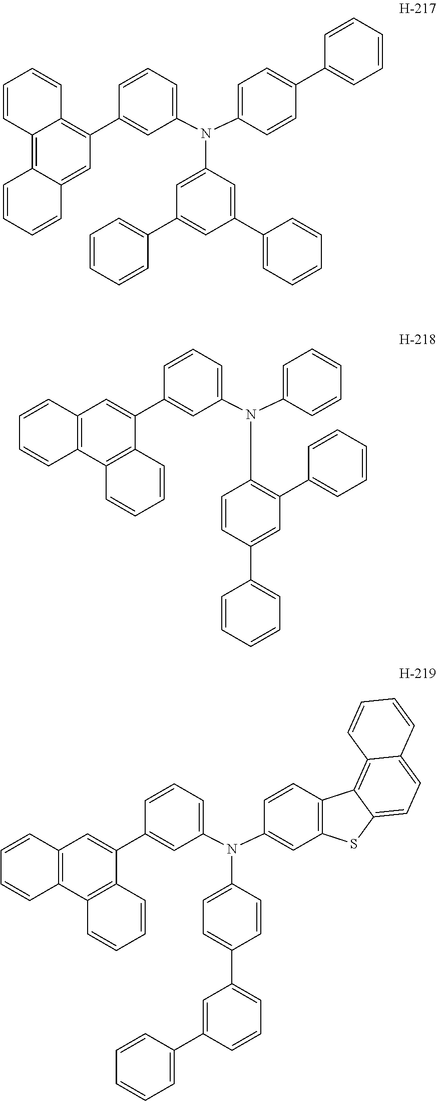 [SMILES: c1ccc(-c2ccc(N(c3cc(-c4ccccc4)cc(-c4ccccc4)c3)c3cccc(-c4cc5ccccc5c5ccccc45)c3)cc2)cc1 ; c1ccc(-c2ccc(N(c3ccccc3)c3cccc(-c4cc5ccccc5c5ccccc45)c3)c(-c3ccccc3)c2)cc1 ; c1ccc(-c2cccc(-c3ccc(N(c4cccc(-c5cc6ccccc6c6ccccc56)c4)c4ccc5c(c4)sc4ccc6ccccc6c45)cc3)c2)cc1]

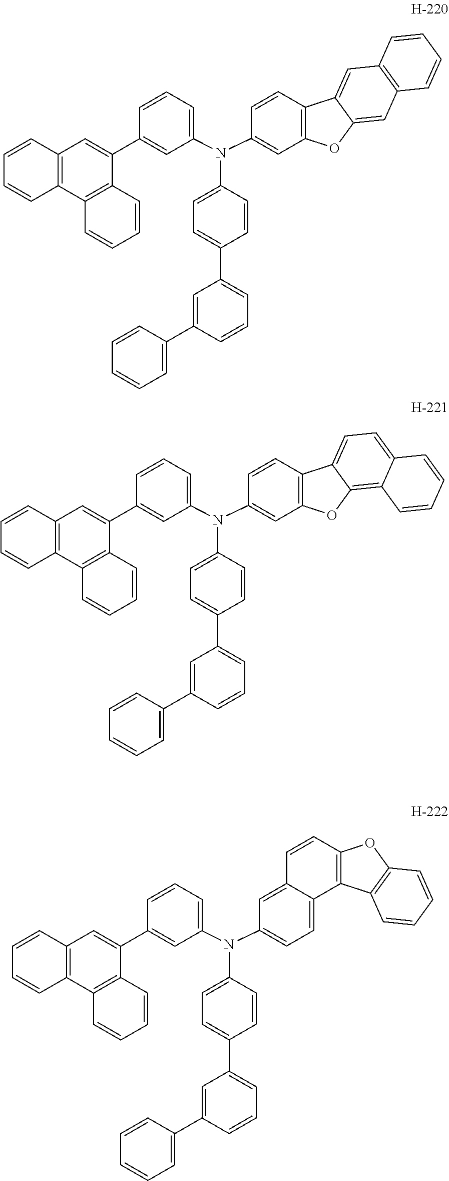 [SMILES: c1ccc(-c2cccc(-c3ccc(N(c4cccc(-c5cc6ccccc6c6ccccc56)c4)c4ccc5c(c4)oc4cc6ccccc6cc45)cc3)c2)cc1 ; c1ccc(-c2cccc(-c3ccc(N(c4cccc(-c5cc6ccccc6c6ccccc56)c4)c4ccc5c(c4)oc4c6ccccc6ccc54)cc3)c2)cc1 ; c1ccc(-c2cccc(-c3ccc(N(c4cccc(-c5cc6ccccc6c6ccccc56)c4)c4ccc5c(ccc6oc7ccccc7c65)c4)cc3)c2)cc1]

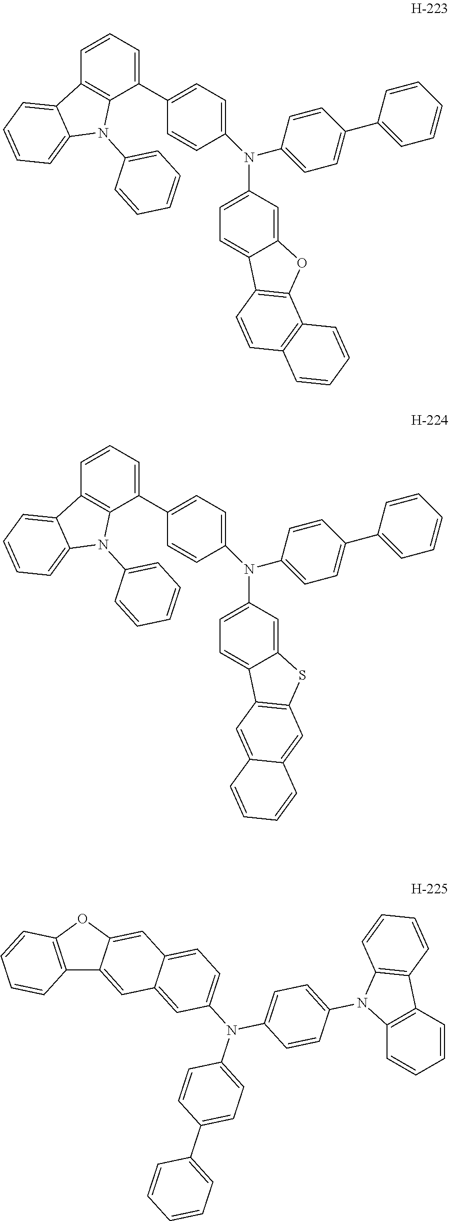 [SMILES: c1ccc(-c2ccc(N(c3ccc(-c4cccc5c6ccccc6n(-c6ccccc6)c45)cc3)c3ccc4c(c3)oc3c5ccccc5ccc43)cc2)cc1 ; c1ccc(-c2ccc(N(c3ccc(-c4cccc5c6ccccc6n(-c6ccccc6)c45)cc3)c3ccc4c(c3)sc3cc5ccccc5cc34)cc2)cc1 ; c1ccc(-c2ccc(N(c3ccc(-n4c5ccccc5c5ccccc54)cc3)c3ccc4cc5oc6ccccc6c5cc4c3)cc2)cc1]

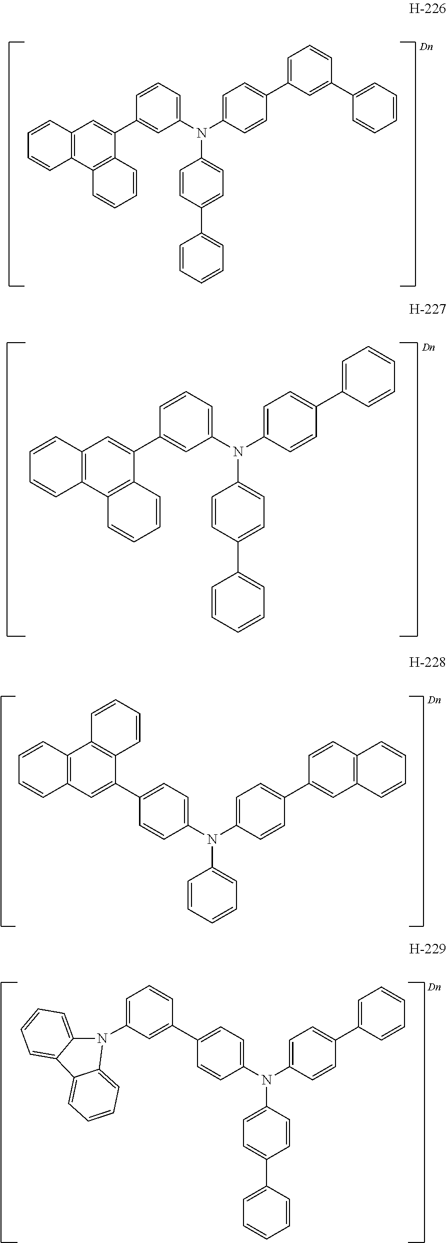

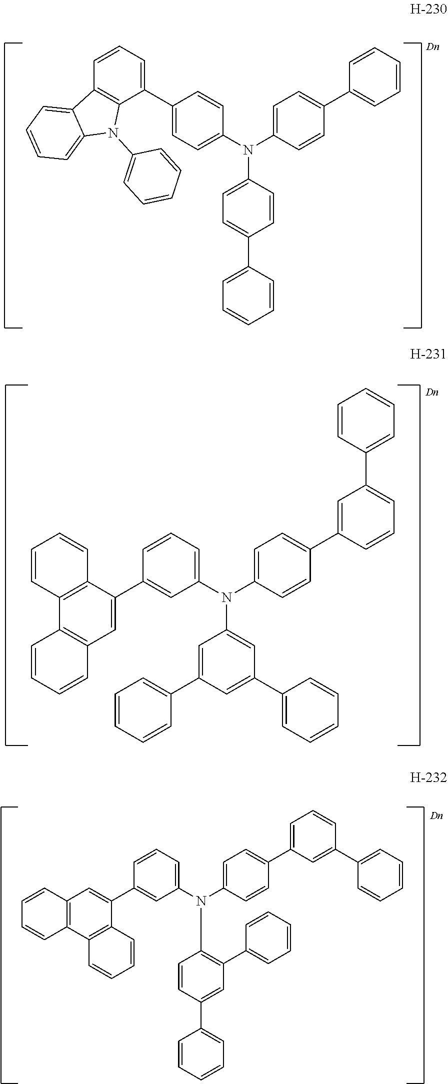

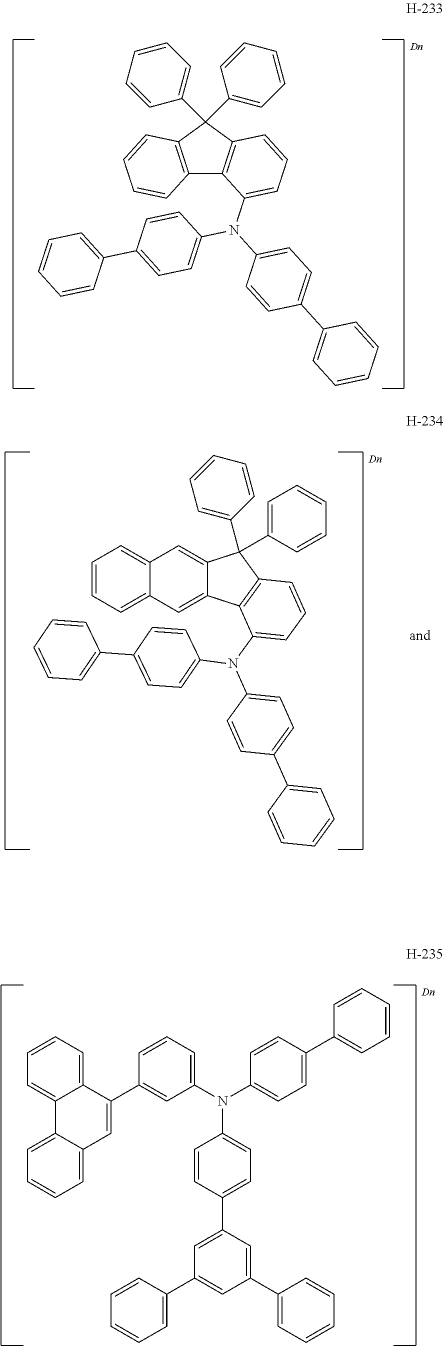

In the compounds above, Dn represents that n number of hydrogens are replaced with deuterium, and n represents an integer of 1 or more, which is an integer of 1 to the maximum number of hydrogens in the compound.

The compound represented by Formula 2 according to the present disclosure may be synthesized by referring to synthetic methods known to one skilled in the art, for example, the methods disclosed in Korean Patent Application No. 2015-0066202 (published on Jun. 16, 2015), Korean Patent Application No. 2015-0076129 (published on Jul. 6, 2015), Korean Patent Application No. 2017-0043439 (published on Apr. 21, 2017), Korean Patent Application No. 2019-0090695 (published on Aug. 2, 2019), Korean Patent Application No. 2020-0004257 (published on Jan. 13, 2020), Korean Patent Application No. 2020-0018275 (published on Feb. 19, 2020), and Korean Patent Application No. 2020-0090091 (published on Jul. 28, 2020), etc., but these are not limited thereto.

In the organic electroluminescent device, the light-emitting layer comprises a light-emitting layer that emits red light.

Hereinafter, an organic electroluminescent compound according to one embodiment of the present disclosure will be described in more detail.

The present disclosure provides an organic electroluminescent compound represented by the following Formula 1.

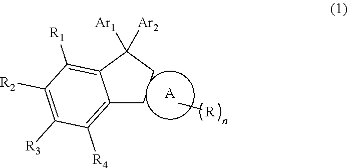

In Formula 1,
ring A represents a substituted or unsubstituted benzene, a substituted or unsubstituted naphthalene, or a substituted or unsubstituted phenanthrene;
Ar₁ and Ar₂ each independently represent a substituted or unsubstituted (C2-C30)alkyl;
R and R₁ to R₄ each independently represent hydrogen, deuterium, a halogen, a cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, or a substituted or unsubstituted (C1-C30)alkoxy, or may be linked to an adjacent substituent(s) to form a ring(s); with a proviso that at least one of R and R₁ to R₄ is represented by -L₁-NAr₃Ar₄;
L₁ represents a single bond, a substituted or unsubstituted (C6-C30)arylene, or a substituted or unsubstituted (3- to 30-membered)heteroarylene;
Ar₃ and Ar₄ each independently represent a substituted or unsubstituted (C6-C30)aryl, or a substituted or unsubstituted (3- to 30-membered)heteroaryl;
n represents an integer of 1 to 8, if n is plural, each of n may be the same as or different from each other.

Formula 1 may be represented by any one of the following Formulas 1-1 to 1-11.

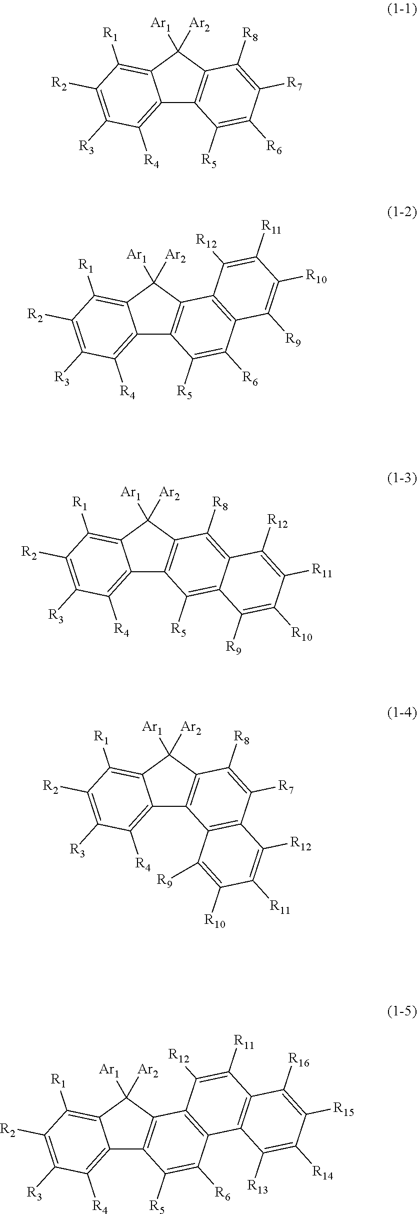

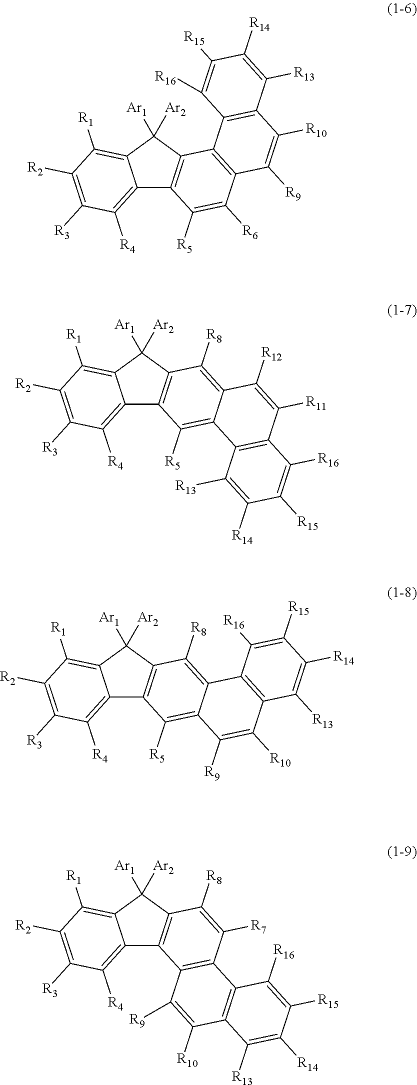

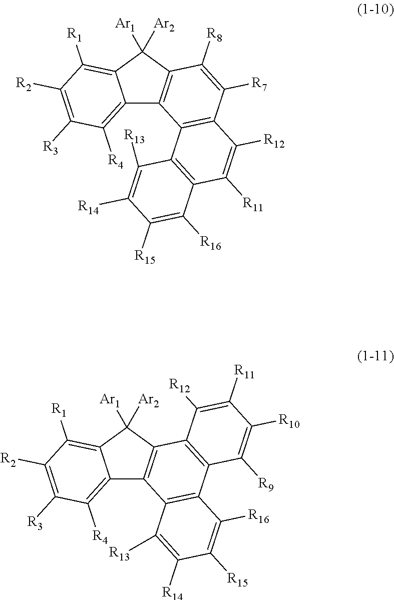

In Formulas 1-1 to 1-11,

Ar₁ and Ar₂ each independently represent a substituted or unsubstituted (C2-C30)alkyl. According to one embodiment of the present disclosure, Ar₁ and Ar₂ each independently may represent a substituted or unsubstituted (C2-C20)alkyl. According to another embodiment of the present disclosure, Ar₁ and Ar₂ each independently may represent a substituted or unsubstituted (C2-C10)alkyl. For example, Ar₁ and Ar₂ each independently may represent a methyl, an ethyl, a propyl, a butyl, a pentyl, a hexyl, a heptyl, or an octyl, and these may be substituted with at least one deuterium.

R₁ to R₁₆ each independently represent hydrogen, deuterium, a halogen, a cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, or a substituted or unsubstituted (C1-C30)alkoxy, or may be linked to an adjacent substituent(s) to form a ring(s); with a proviso that at least one of R₁ to R₁₆ is represented by -L₁-NAr₃Ar₄. According to one embodiment of the present disclosure, R₁ to R₁₆ each independently represent hydrogen, deuterium, a substituted or unsubstituted (C1-C20)alkyl, a substituted or unsubstituted (C6-C20)aryl, or may be linked to an adjacent substituent(s) to form a ring(s); with a proviso that at least one of R₁ to R₁₆ is represented by -L₁-NAr₃Ar₄. For example, R₁ to R₁₆ each independently represent hydrogen, deuterium, or -L₁-NAr₃Ar₄.

L₁ represents a single bond, a substituted or unsubstituted (C6-C30)arylene, or a substituted or unsubstituted (3- to 30-membered)heteroarylene. According to one embodiment of the present disclosure, L₁ may represent a single bond, a substituted or unsubstituted (C6-C25)arylene, or a substituted or unsubstituted (3- to 25-membered)heteroarylene. For example, L₁ may represent a single bond, or a phenylene.

Ar₃ and Ar₄ each independently represent a substituted or unsubstituted (C6-C30)aryl, or a substituted or unsubstituted (3- to 30-membered)heteroaryl. According to one embodiment of the present disclosure, Ar₃ and Ar₄ each independently may represent a substituted or unsubstituted (C6-C30)aryl, or a substituted or unsubstituted (3- to 25-membered)heteroaryl. For example, Ar₃ and Ar₄ each independently may represent a benzonaphthofuranyl; a benzonaphthothiophenyl; a biphenyl; a dibenzofuranyl; a dibenzothiophenyl; a dimethylfluorenyl; a diethylfluorenyl; a diphenylfluorenyl; a dimethylbenzofluorenyl; a diethylbenzofluorenyl; a dipropylfluorenyl; a dibutylfluorenyl; a dipentylfluorenyl; a dihexylfluorenyl; a diheptylfluorenyl; a dioctylfluorenyl; a diphenylbenzofluorenyl; a naphthyl; a phenanthrenyl substituted or unsubstituted with a naphthyl, a phenyl, a phenyl substituted with deuterium, a dibenzofuranyl, or a phenyldimethylfluorenylamino; a phenyl substituted or unsubstituted with a methyl, a naphthyl, a benzonaphthofuranyl, or a benzonaphthothiophenyl; a quaterphenyl; an o-terphenyl; or a p-terphenyl, and these may be substituted with at least one deuterium.

Ar₃ in Formula 1 is represented by any one of the following Formulas.

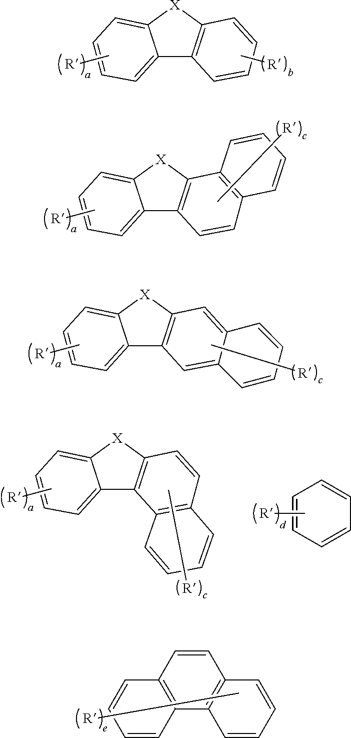

In the above Formulas,

X represents O, S, NR₇₃, or CR₇₄R₇₅. For example, X may represent O, S, or CR₇₄R₇₅.

R′ and R₇₃ to R₇₅ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di(C1-C30)alkylamino, a substituted or unsubstituted mono- or di(C6-C30)arylamino, or a substituted or unsubstituted (C1-C30)alkyl(C6-C30)arylamino, or may be linked to an adjacent substituent(s) to form a substituted or unsubstituted (3- to 30-membered) monocyclic or polycyclic ring(s). According to one embodiment of the present disclosure, R′ and R₇₃ to R₇₅ each independently represent hydrogen, deuterium, a substituted or unsubstituted (C1-C30)alkyl, a substituted or unsubstituted (C6-C30)aryl. According to another embodiment of the present disclosure, R′ and R₇₃ to R₇₅ each independently may represent hydrogen, deuterium, a substituted or unsubstituted (C1-C20)alkyl, a substituted or unsubstituted (C6-C20)aryl. For example, R′ and R₇₃ to R₇₅ each independently may represent hydrogen; deuterium; a methyl substituted or unsubstituted with deuterium; an ethyl substituted or unsubstituted with deuterium; a propyl substituted or unsubstituted with deuterium; a butyl substituted or unsubstituted with deuterium; a pentyl substituted or unsubstituted with deuterium; a hexyl substituted or unsubstituted with deuterium; a heptyl substituted or unsubstituted with deuterium; an octyl substituted or unsubstituted with deuterium; or a phenyl substituted or unsubstituted with deuterium.

In each Formula, any one of R′ is linked to L₁ of Formula 1.

In each Formula, a and b each independently represent an integer of 1 to 4, c and d each independently represent an integer of 1 to 6, e represents an integer of 1 to 10, if a to e represent an integer of 2 or more, each of R′ may be the same as or different from each other.

The compound represented by Formula 1 may be at least one selected from the following compounds, but is not limited thereto.

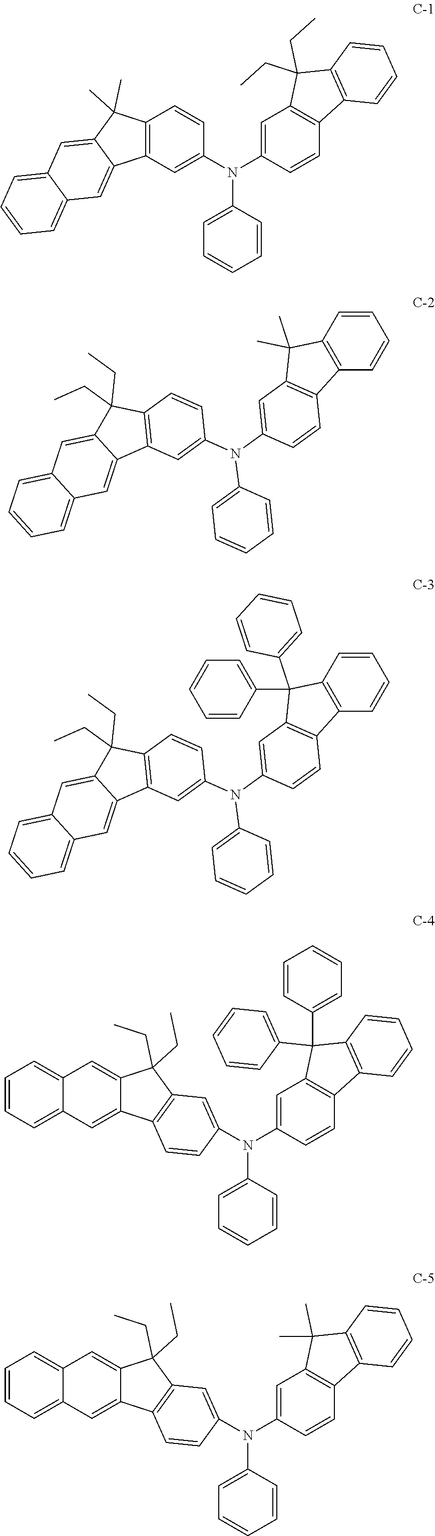

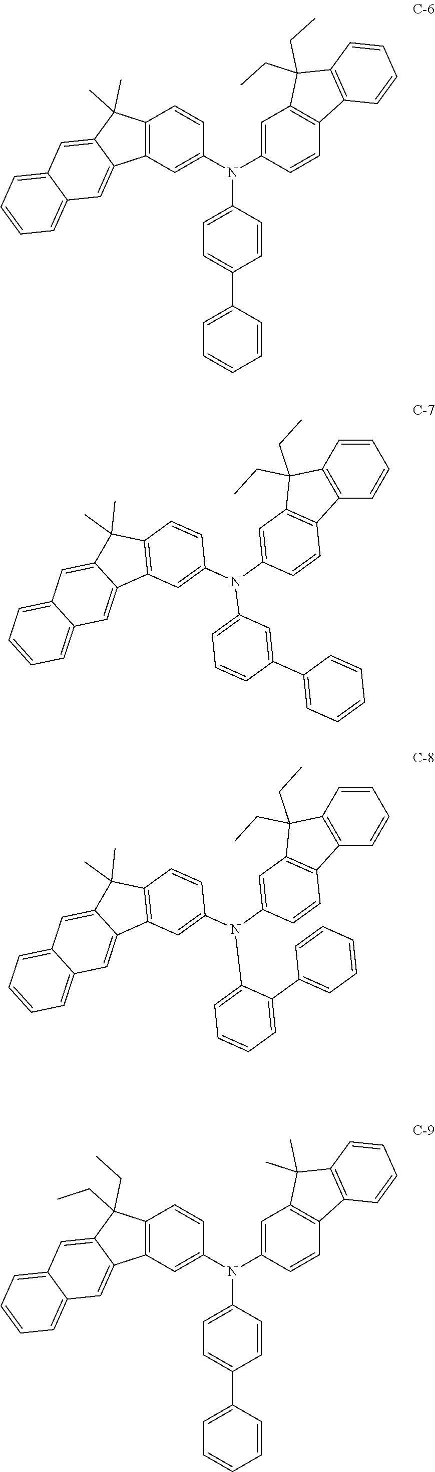 [SMILES: CCC1(CC)c2ccccc2-c2ccc(N(c3ccc(-c4ccccc4)cc3)c3ccc4c(c3)-c3cc5ccccc5cc3C4(C)C)cc21 ; CCC1(CC)c2ccccc2-c2ccc(N(c3cccc(-c4ccccc4)c3)c3ccc4c(c3)-c3cc5ccccc5cc3C4(C)C)cc21 ; CCC1(CC)c2ccccc2-c2ccc(N(c3ccc4c(c3)-c3cc5ccccc5cc3C4(C)C)c3ccccc3-c3ccccc3)cc21 ; CCC1(CC)c2ccc(N(c3ccc(-c4ccccc4)cc3)c3ccc4c(c3)C(C)(C)c3ccccc3-4)cc2-c2cc3ccccc3cc21]

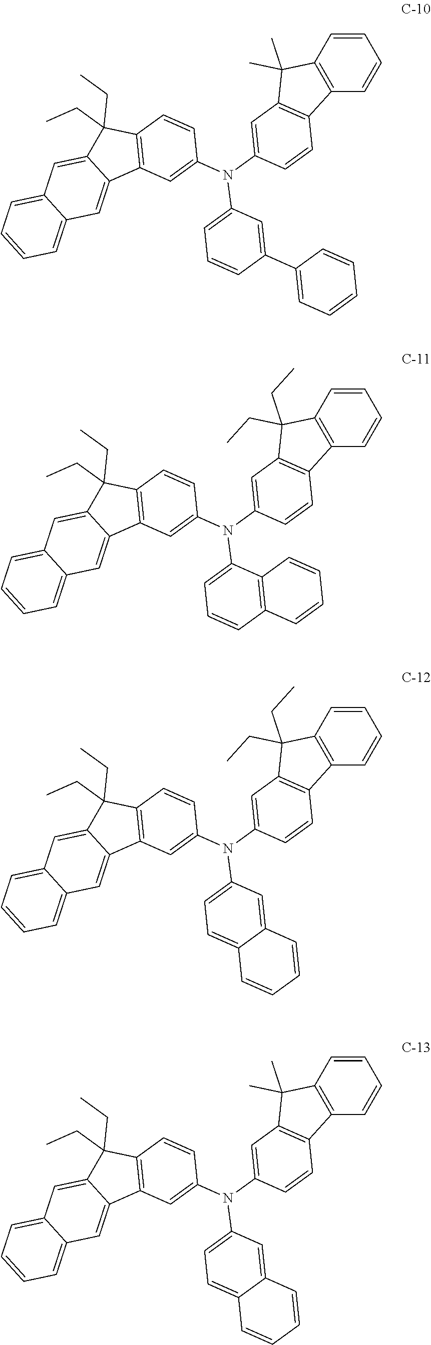 [SMILES: CCC1(CC)c2ccc(N(c3cccc(-c4ccccc4)c3)c3ccc4c(c3)C(C)(C)c3ccccc3-4)cc2-c2cc3ccccc3cc21 ; CCC1(CC)c2ccccc2-c2ccc(N(c3ccc4c(c3)-c3cc5ccccc5cc3C4(CC)CC)c3cccc4ccccc34)cc21 ; CCC1(CC)c2ccccc2-c2ccc(N(c3ccc4c(c3)-c3cc5ccccc5cc3C4(CC)CC)c3ccc4ccccc4c3)cc21 ; CCC1(CC)c2ccc(N(c3ccc4c(c3)C(C)(C)c3ccccc3-4)c3ccc4ccccc4c3)cc2-c2cc3ccccc3cc21]

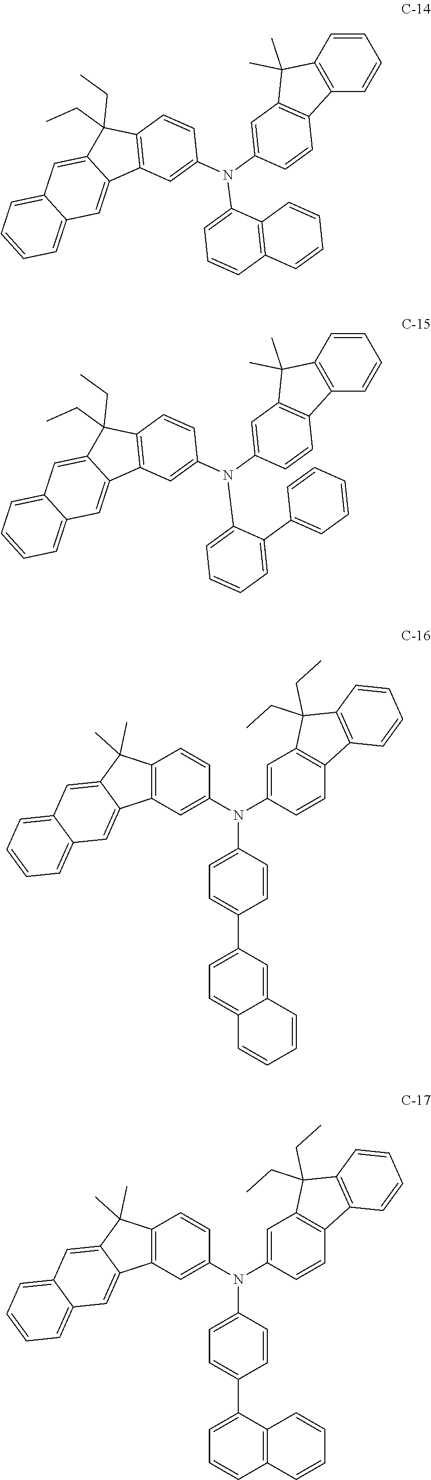 [SMILES: CCC1(CC)c2ccc(N(c3ccc4c(c3)C(C)(C)c3ccccc3-4)c3cccc4ccccc34)cc2-c2cc3ccccc3cc21 ; CCC1(CC)c2ccc(N(c3ccc4c(c3)C(C)(C)c3ccccc3-4)c3ccccc3-c3ccccc3)cc2-c2cc3ccccc3cc21 ; CCC1(CC)c2ccccc2-c2ccc(N(c3ccc(-c4ccc5ccccc5c4)cc3)c3ccc4c(c3)-c3cc5ccccc5cc3C4(C)C)cc21 ; CCC1(CC)c2ccccc2-c2ccc(N(c3ccc(-c4cccc5ccccc45)cc3)c3ccc4c(c3)-c3cc5ccccc5cc3C4(C)C)cc21]

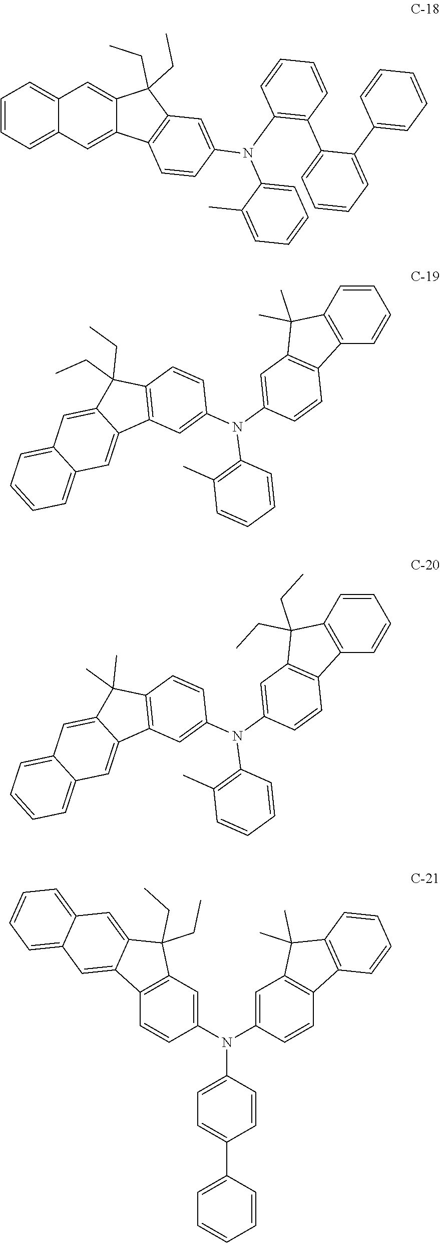

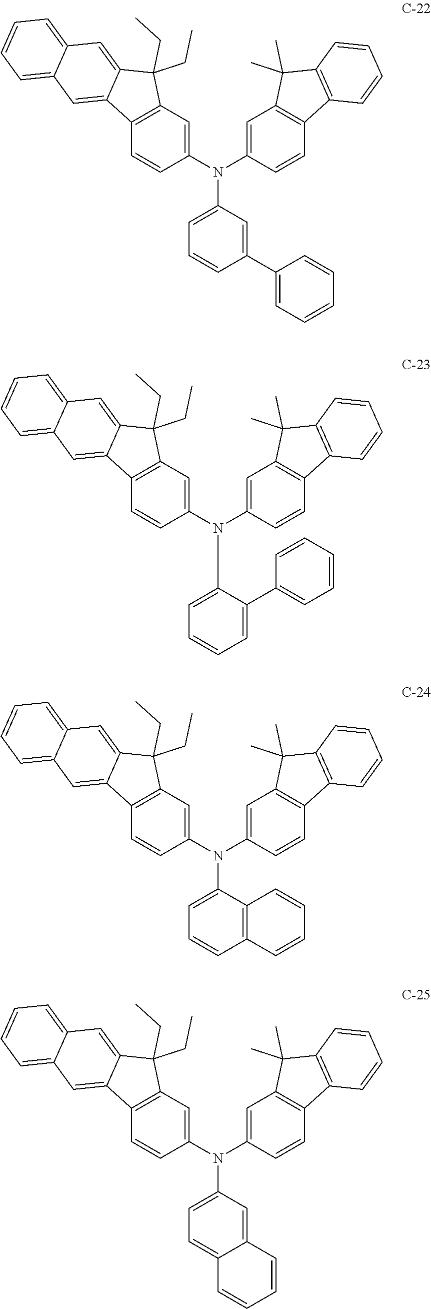 [SMILES: CCC1(CC)c2cc(N(c3cccc(-c4ccccc4)c3)c3ccc4c(c3)C(C)(C)c3ccccc3-4)ccc2-c2cc3ccccc3cc21 ; CCC1(CC)c2cc(N(c3ccc4c(c3)C(C)(C)c3ccccc3-4)c3ccccc3-c3ccccc3)ccc2-c2cc3ccccc3cc21 ; CCC1(CC)c2cc(N(c3ccc4c(c3)C(C)(C)c3ccccc3-4)c3cccc4ccccc34)ccc2-c2cc3ccccc3cc21 ; CCC1(CC)c2cc(N(c3ccc4c(c3)C(C)(C)c3ccccc3-4)c3ccc4ccccc4c3)ccc2-c2cc3ccccc3cc21]

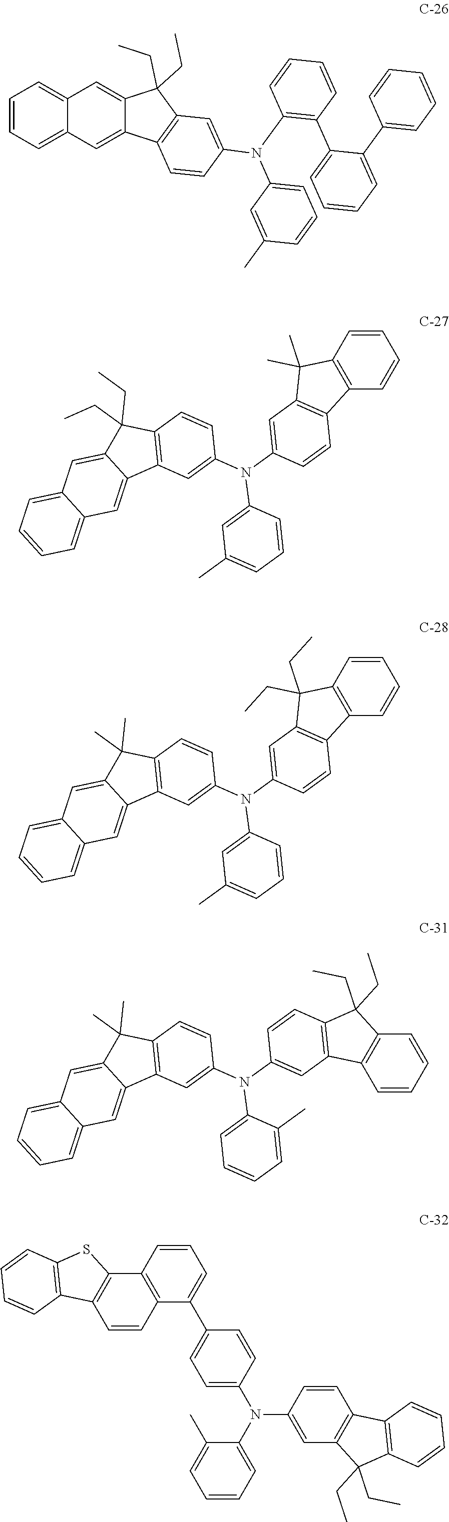

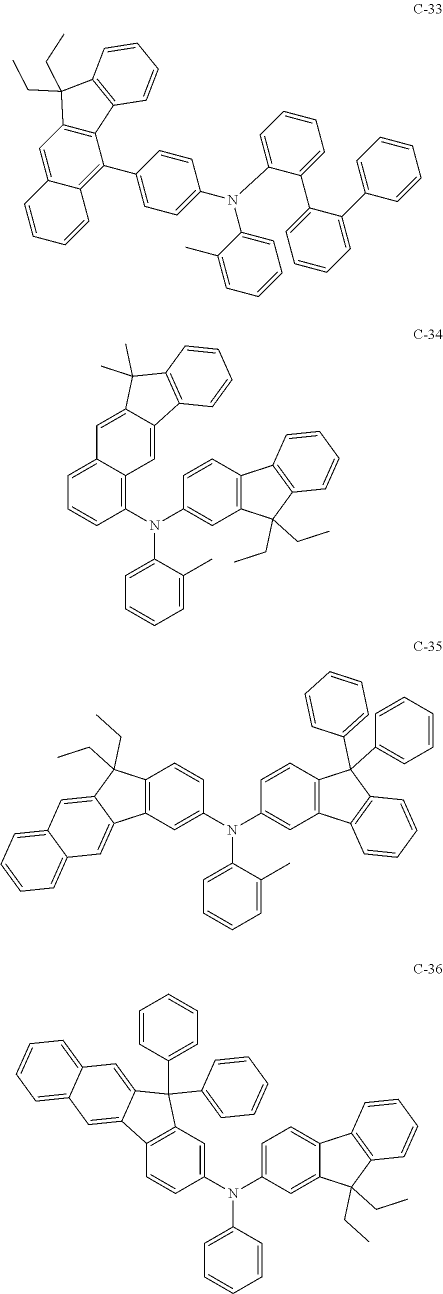 [SMILES: CCC1(CC)c2ccccc2-c2c1cc1ccccc1c2-c1ccc(N(c2ccccc2C)c2ccccc2-c2ccccc2-c2ccccc2)cc1 ; CCC1(CC)c2ccccc2-c2ccc(N(c3ccccc3C)c3cccc4cc5c(cc34)-c3ccccc3C5(C)C)cc21 ; CCC1(CC)c2ccc(N(c3ccc4c(c3)-c3ccccc3C4(c3ccccc3)c3ccccc3)c3ccccc3C)cc2-c2cc3ccccc3cc21 ; CCC1(CC)c2ccccc2-c2ccc(N(c3ccccc3)c3ccc4c(c3)C(c3ccccc3)(c3ccccc3)c3cc5ccccc5cc3-4)cc21]

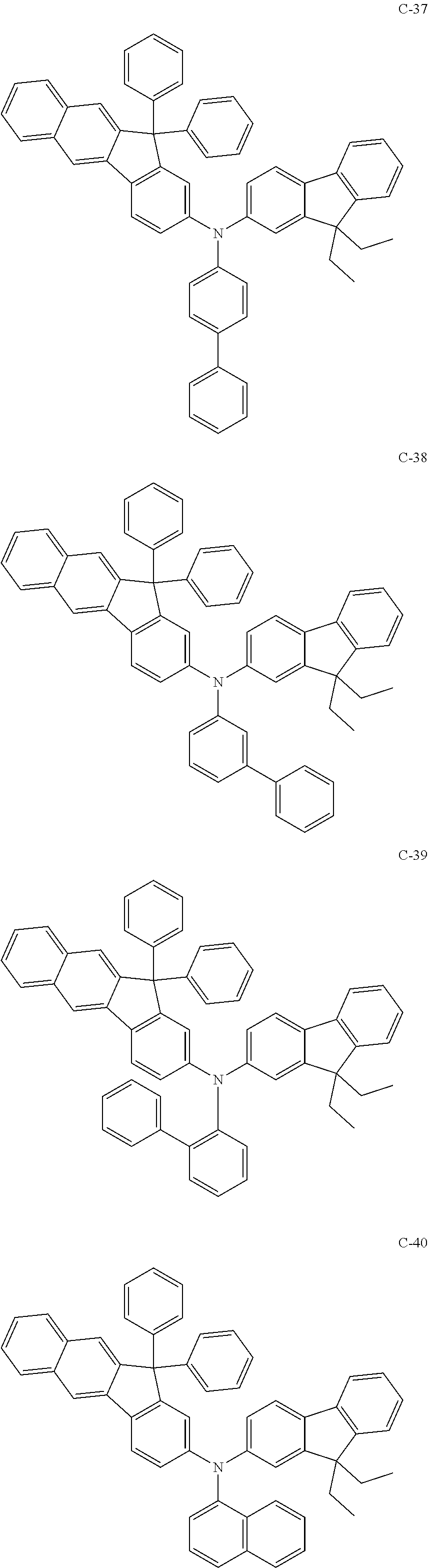 [SMILES: CCC1(CC)c2ccccc2-c2ccc(N(c3ccc(-c4ccccc4)cc3)c3ccc4c(c3)C(c3ccccc3)(c3ccccc3)c3cc5ccccc5cc3-4)cc21 ; CCC1(CC)c2ccccc2-c2ccc(N(c3cccc(-c4ccccc4)c3)c3ccc4c(c3)C(c3ccccc3)(c3ccccc3)c3cc5ccccc5cc3-4)cc21 ; CCC1(CC)c2ccccc2-c2ccc(N(c3ccc4c(c3)C(c3ccccc3)(c3ccccc3)c3cc5ccccc5cc3-4)c3ccccc3-c3ccccc3)cc21 ; CCC1(CC)c2ccccc2-c2ccc(N(c3ccc4c(c3)C(c3ccccc3)(c3ccccc3)c3cc5ccccc5cc3-4)c3cccc4ccccc34)cc21]

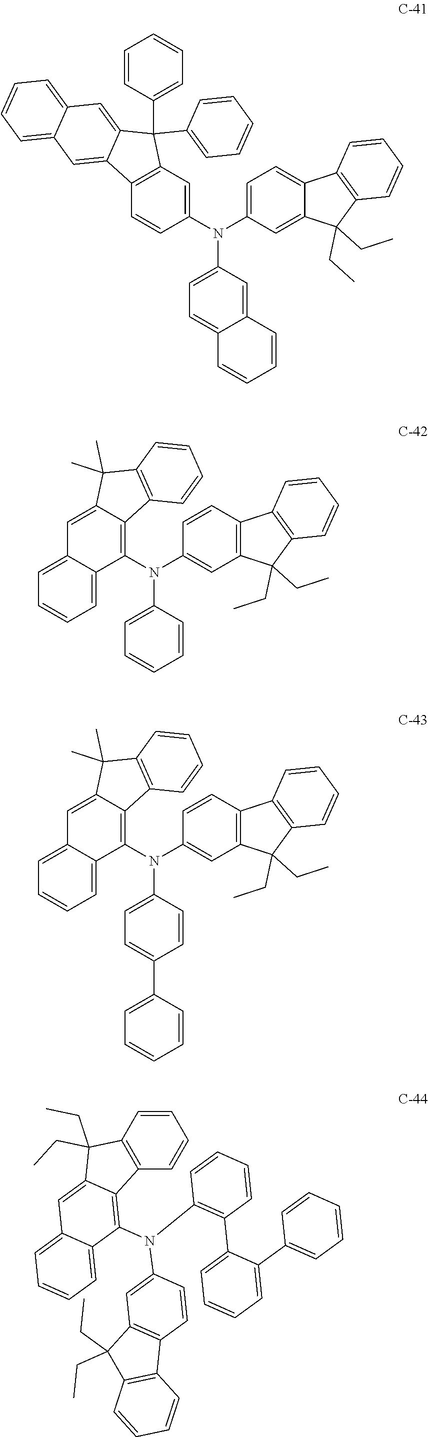 [SMILES: CCC1(CC)c2ccccc2-c2ccc(N(c3ccc4c(c3)C(c3ccccc3)(c3ccccc3)c3cc5ccccc5cc3-4)c3ccc4ccccc4c3)cc21 ; CCC1(CC)c2ccccc2-c2ccc(N(c3ccccc3)c3c4c(cc5ccccc35)C(C)(C)c3ccccc3-4)cc21 ; CCC1(CC)c2ccccc2-c2ccc(N(c3ccc(-c4ccccc4)cc3)c3c4c(cc5ccccc35)C(C)(C)c3ccccc3-4)cc21 ; CCC1(CC)c2ccccc2-c2ccc(N(c3ccccc3-c3ccccc3-c3ccccc3)c3c4c(cc5ccccc35)C(CC)(CC)c3ccccc3-4)cc21]

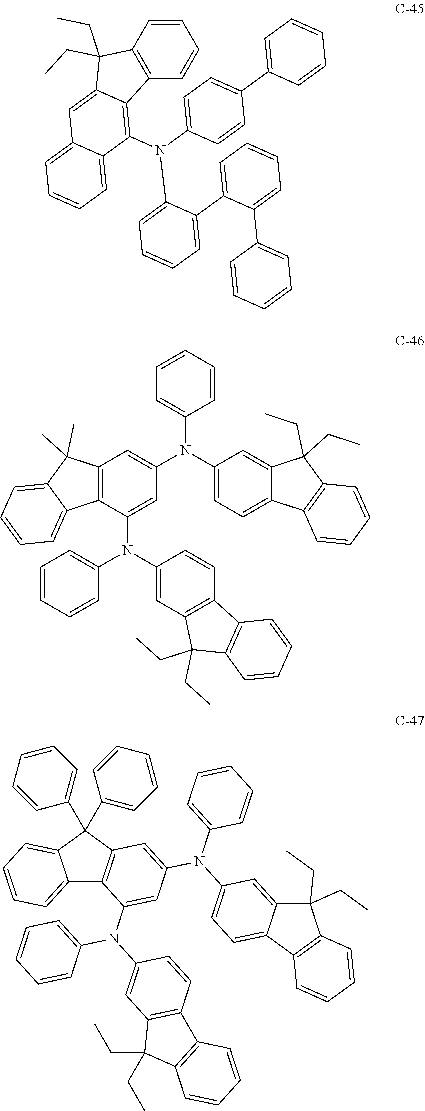 [SMILES: CCC1(CC)c2ccccc2-c2c1cc1ccccc1c2N(c1ccc(-c2ccccc2)cc1)c1ccccc1-c1ccccc1-c1ccccc1 ; CCC1(CC)c2ccccc2-c2ccc(N(c3ccccc3)c3cc(N(c4ccccc4)c4ccc5c(c4)C(CC)(CC)c4ccccc4-5)c4c(c3)C(C)(C)c3ccccc3-4)cc21 ; CCC1(CC)c2ccccc2-c2ccc(N(c3ccccc3)c3cc(N(c4ccccc4)c4ccc5c(c4)C(CC)(CC)c4ccccc4-5)c4c(c3)C(c3ccccc3)(c3ccccc3)c3ccccc3-4)cc21]

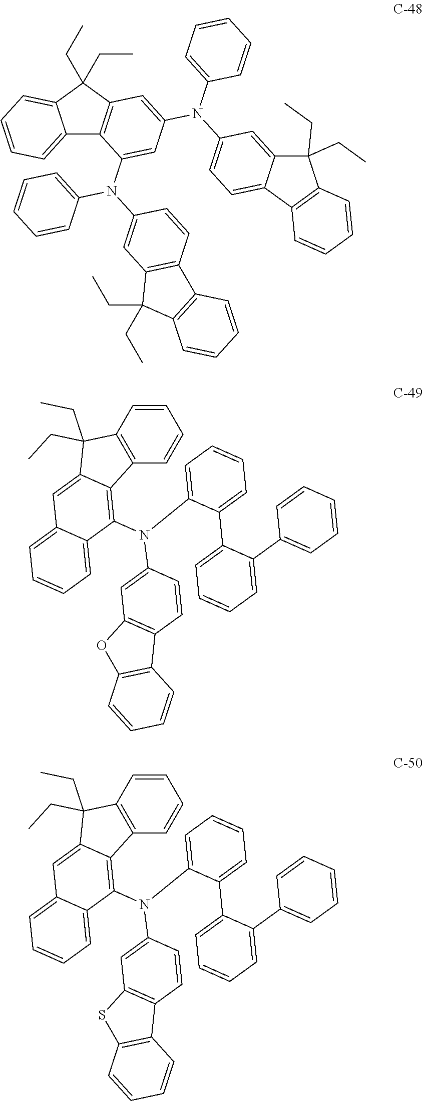

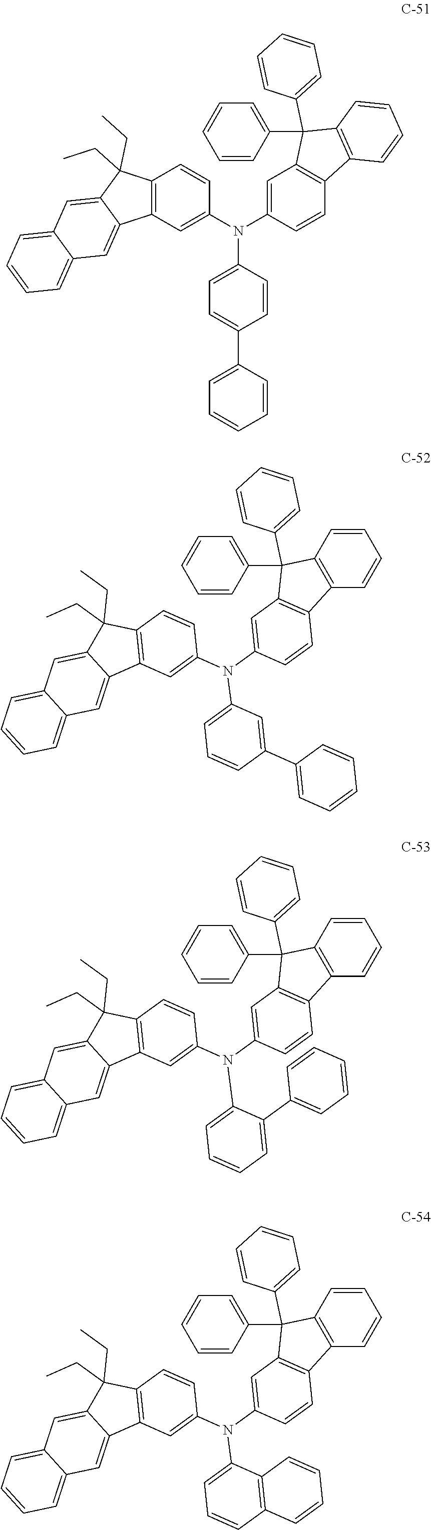

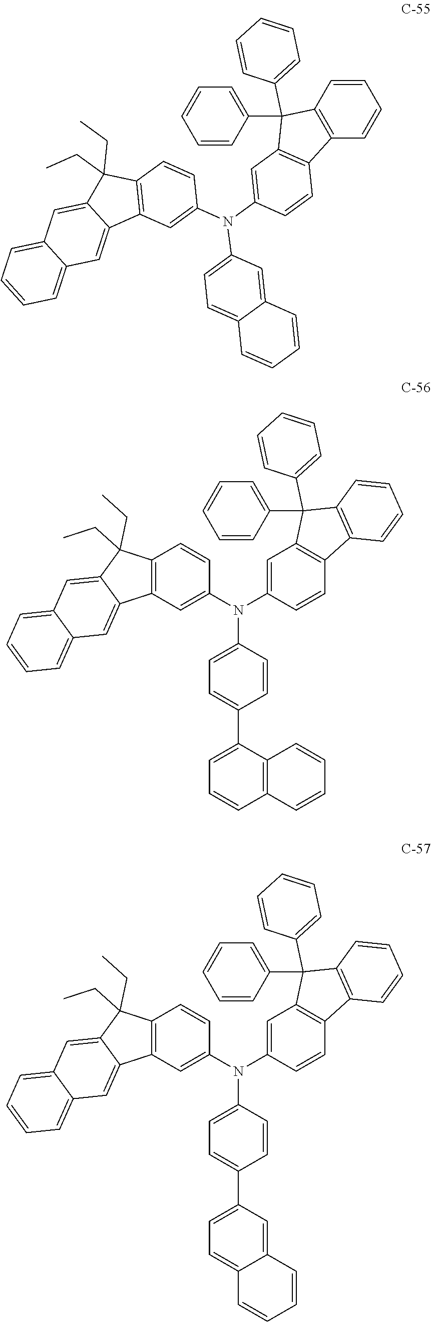

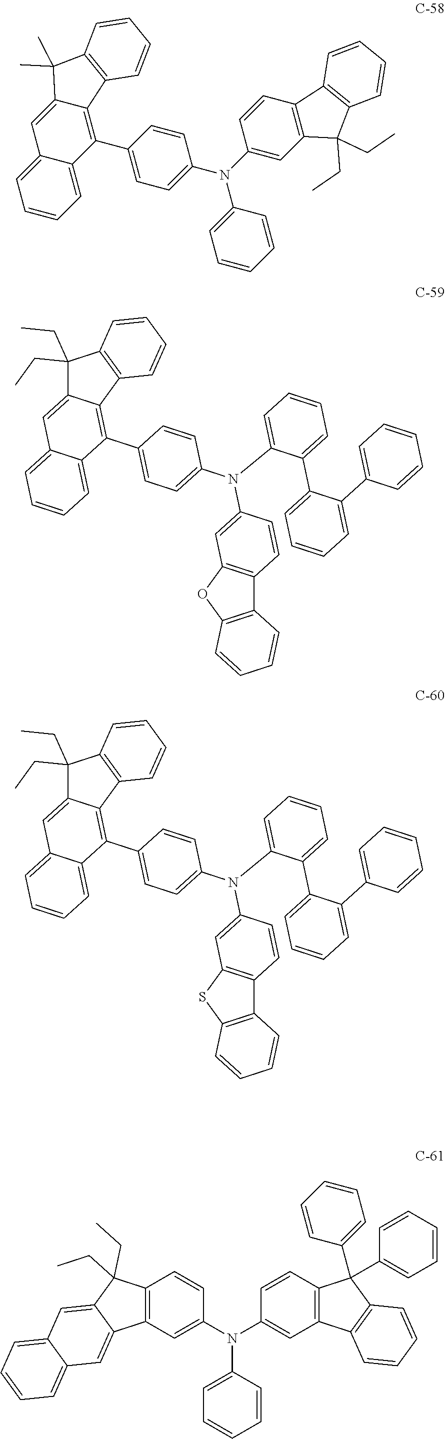

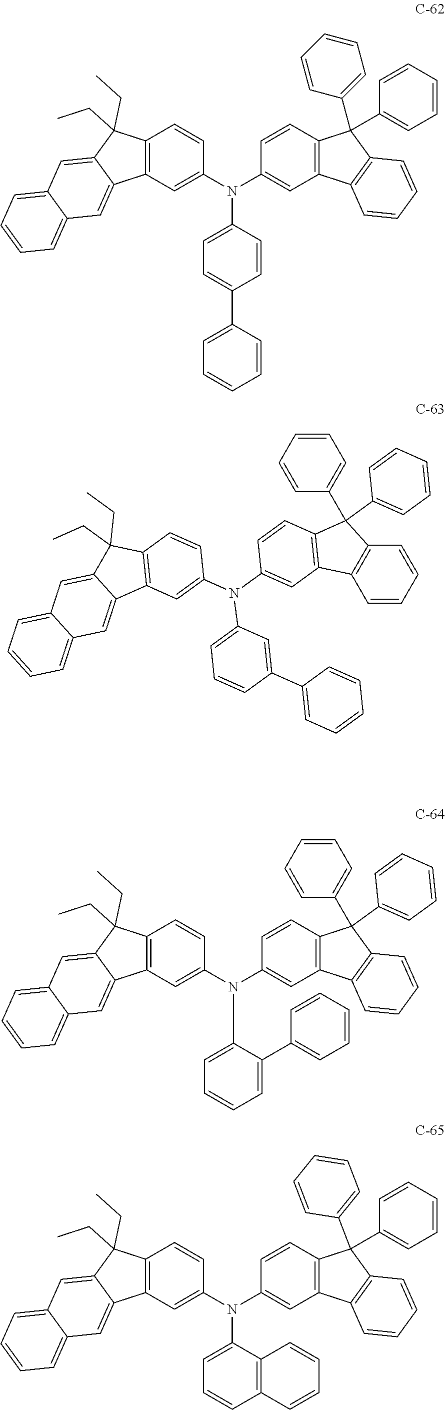 [SMILES: CCC1(CC)c2ccc(N(c3ccc(-c4ccccc4)cc3)c3ccc4c(c3)-c3ccccc3C4(c3ccccc3)c3ccccc3)cc2-c2cc3ccccc3cc21 ; CCC1(CC)c2ccc(N(c3cccc(-c4ccccc4)c3)c3ccc4c(c3)-c3ccccc3C4(c3ccccc3)c3ccccc3)cc2-c2cc3ccccc3cc21 ; CCC1(CC)c2ccc(N(c3ccc4c(c3)-c3ccccc3C4(c3ccccc3)c3ccccc3)c3ccccc3-c3ccccc3)cc2-c2cc3ccccc3cc21 ; CCC1(CC)c2ccc(N(c3ccc4c(c3)-c3ccccc3C4(c3ccccc3)c3ccccc3)c3cccc4ccccc34)cc2-c2cc3ccccc3cc21]

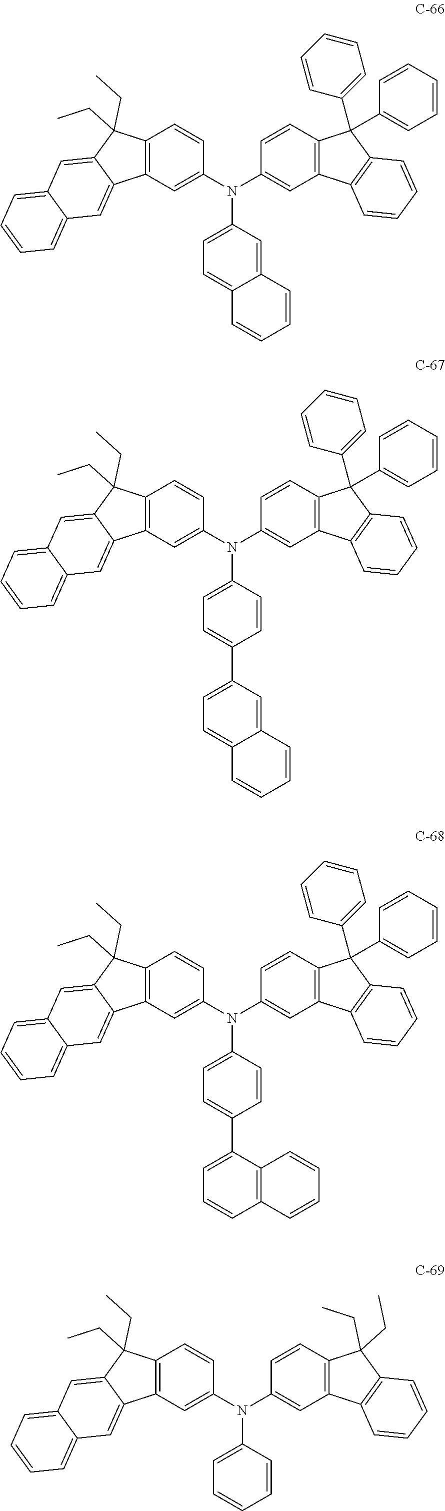 [SMILES: CCC1(CC)c2ccc(N(c3ccc4c(c3)-c3ccccc3C4(c3ccccc3)c3ccccc3)c3ccc4ccccc4c3)cc2-c2cc3ccccc3cc21 ; CCC1(CC)c2ccc(N(c3ccc(-c4ccc5ccccc5c4)cc3)c3ccc4c(c3)-c3ccccc3C4(c3ccccc3)c3ccccc3)cc2-c2cc3ccccc3cc21 ; CCC1(CC)c2ccc(N(c3ccc(-c4cccc5ccccc45)cc3)c3ccc4c(c3)-c3ccccc3C4(c3ccccc3)c3ccccc3)cc2-c2cc3ccccc3cc21 ; CCC1(CC)c2ccccc2-c2cc(N(c3ccccc3)c3ccc4c(c3)-c3cc5ccccc5cc3C4(CC)CC)ccc21]

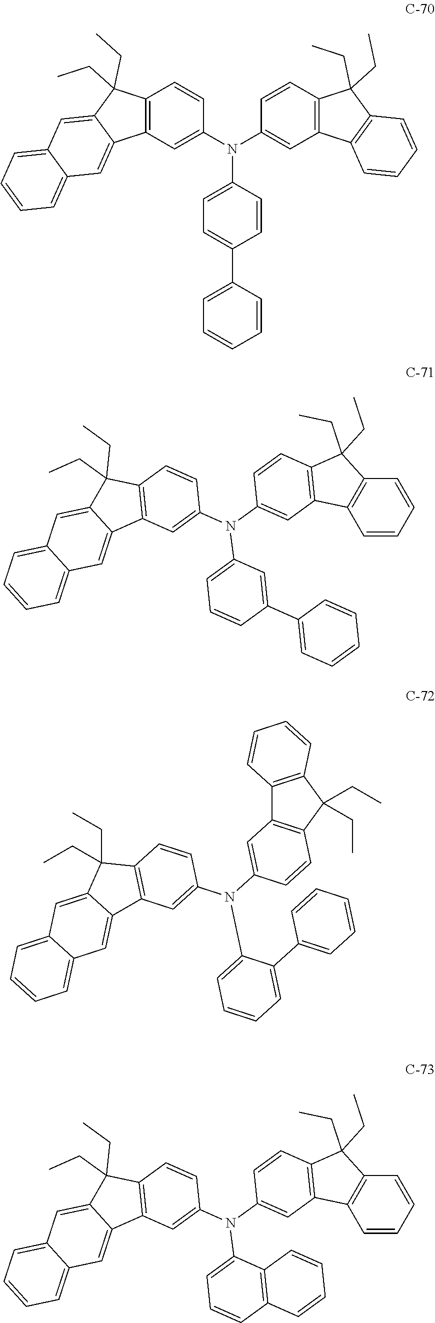 [SMILES: CCC1(CC)c2ccccc2-c2cc(N(c3ccc(-c4ccccc4)cc3)c3ccc4c(c3)-c3cc5ccccc5cc3C4(CC)CC)ccc21 ; CCC1(CC)c2ccccc2-c2cc(N(c3cccc(-c4ccccc4)c3)c3ccc4c(c3)-c3cc5ccccc5cc3C4(CC)CC)ccc21 ; CCC1(CC)c2ccccc2-c2cc(N(c3ccc4c(c3)-c3cc5ccccc5cc3C4(CC)CC)c3ccccc3-c3ccccc3)ccc21 ; CCC1(CC)c2ccccc2-c2cc(N(c3ccc4c(c3)-c3cc5ccccc5cc3C4(CC)CC)c3cccc4ccccc34)ccc21]

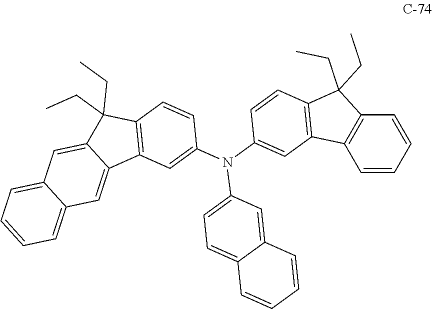 [SMILES: CCC1(CC)c2ccccc2-c2cc(N(c3ccc4c(c3)-c3cc5ccccc5cc3C4(CC)CC)c3ccc4ccccc4c3)ccc21]

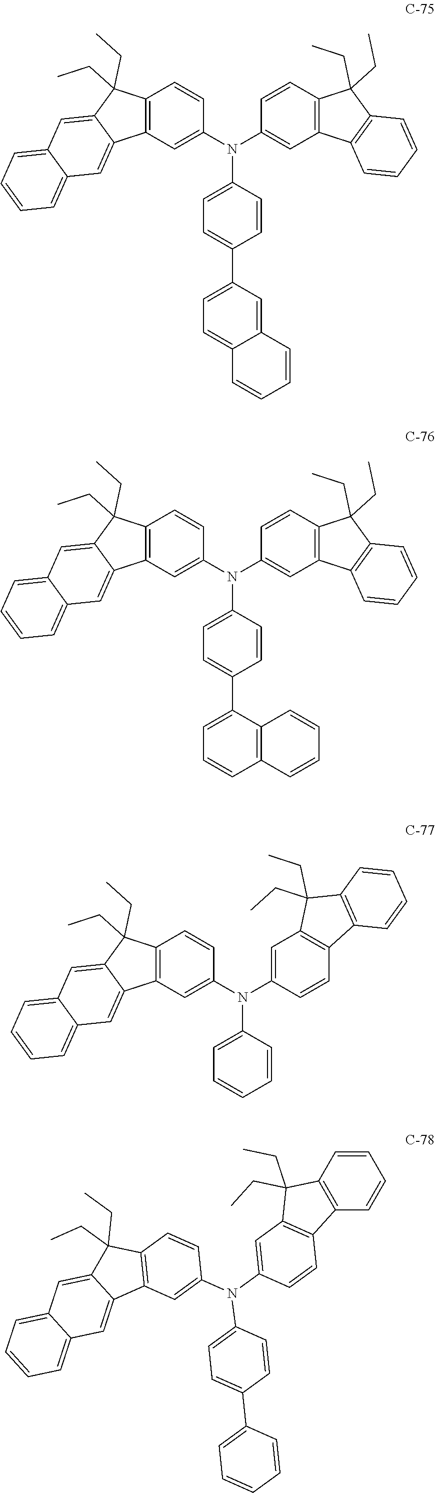 [SMILES: CCC1(CC)c2ccccc2-c2cc(N(c3ccc(-c4ccc5ccccc5c4)cc3)c3ccc4c(c3)-c3cc5ccccc5cc3C4(CC)CC)ccc21 ; CCC1(CC)c2ccccc2-c2cc(N(c3ccc(-c4cccc5ccccc45)cc3)c3ccc4c(c3)-c3cc5ccccc5cc3C4(CC)CC)ccc21 ; CCC1(CC)c2ccccc2-c2ccc(N(c3ccccc3)c3ccc4c(c3)-c3cc5ccccc5cc3C4(CC)CC)cc21 ; CCC1(CC)c2ccccc2-c2ccc(N(c3ccc(-c4ccccc4)cc3)c3ccc4c(c3)-c3cc5ccccc5cc3C4(CC)CC)cc21]

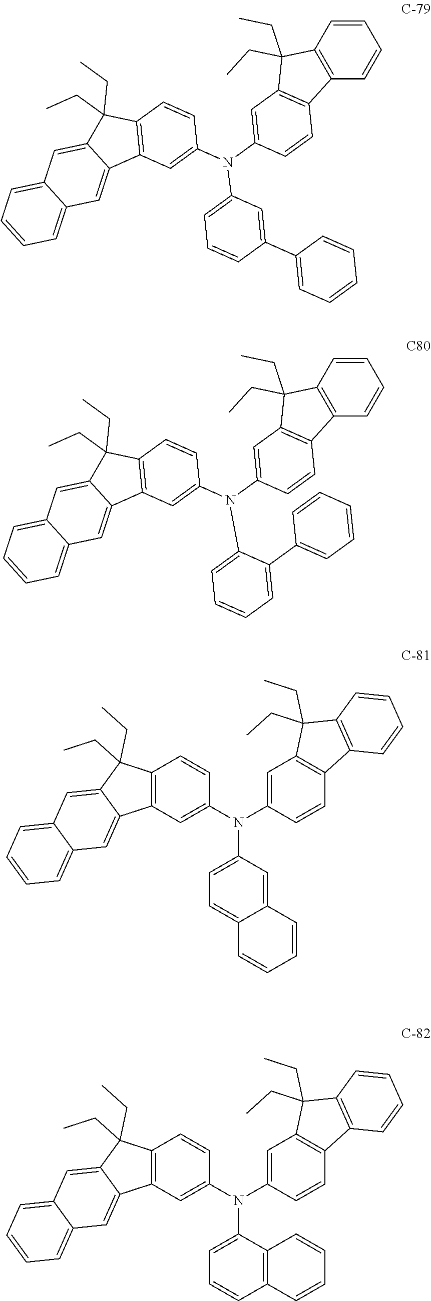 [SMILES: CCC1(CC)c2ccccc2-c2ccc(N(c3cccc(-c4ccccc4)c3)c3ccc4c(c3)-c3cc5ccccc5cc3C4(CC)CC)cc21 ; CCC1(CC)c2ccccc2-c2ccc(N(c3ccc4c(c3)-c3cc5ccccc5cc3C4(CC)CC)c3ccccc3-c3ccccc3)cc21 ; CCC1(CC)c2ccccc2-c2ccc(N(c3ccc4c(c3)-c3cc5ccccc5cc3C4(CC)CC)c3ccc4ccccc4c3)cc21 ; CCC1(CC)c2ccccc2-c2ccc(N(c3ccc4c(c3)-c3cc5ccccc5cc3C4(CC)CC)c3cccc4ccccc34)cc21]

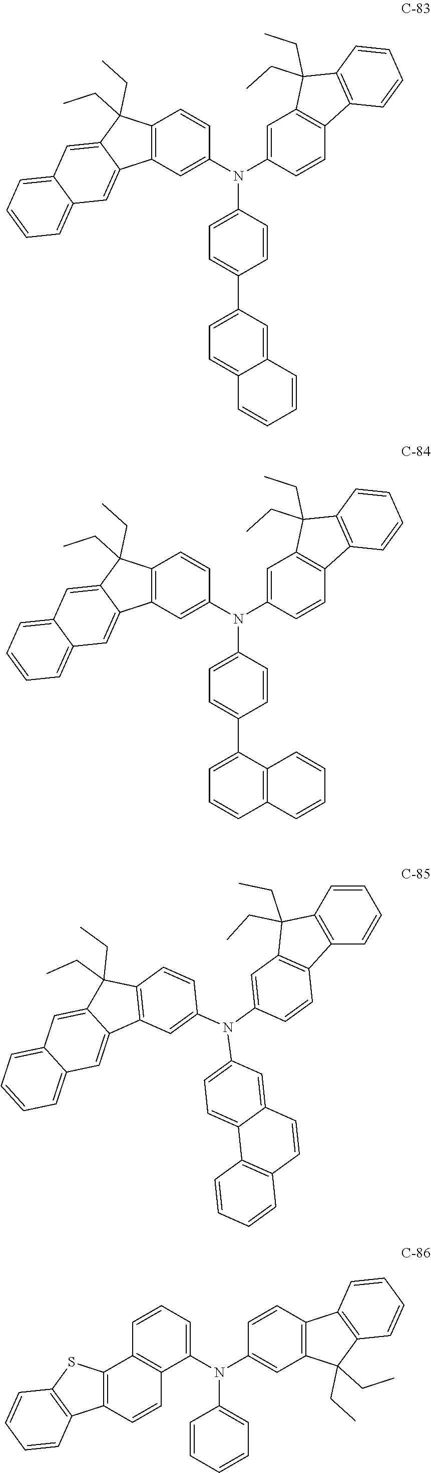 [SMILES: CCC1(CC)c2ccccc2-c2ccc(N(c3ccc(-c4ccc5ccccc5c4)cc3)c3ccc4c(c3)-c3cc5ccccc5cc3C4(CC)CC)cc21 ; CCC1(CC)c2ccccc2-c2ccc(N(c3ccc(-c4cccc5ccccc45)cc3)c3ccc4c(c3)-c3cc5ccccc5cc3C4(CC)CC)cc21 ; CCC1(CC)c2ccccc2-c2ccc(N(c3ccc4c(c3)-c3cc5ccccc5cc3C4(CC)CC)c3ccc4c(ccc5ccccc54)c3)cc21 ; CCC1(CC)c2ccccc2-c2ccc(N(c3ccccc3)c3cccc4c3ccc3c5ccccc5sc43)cc21]

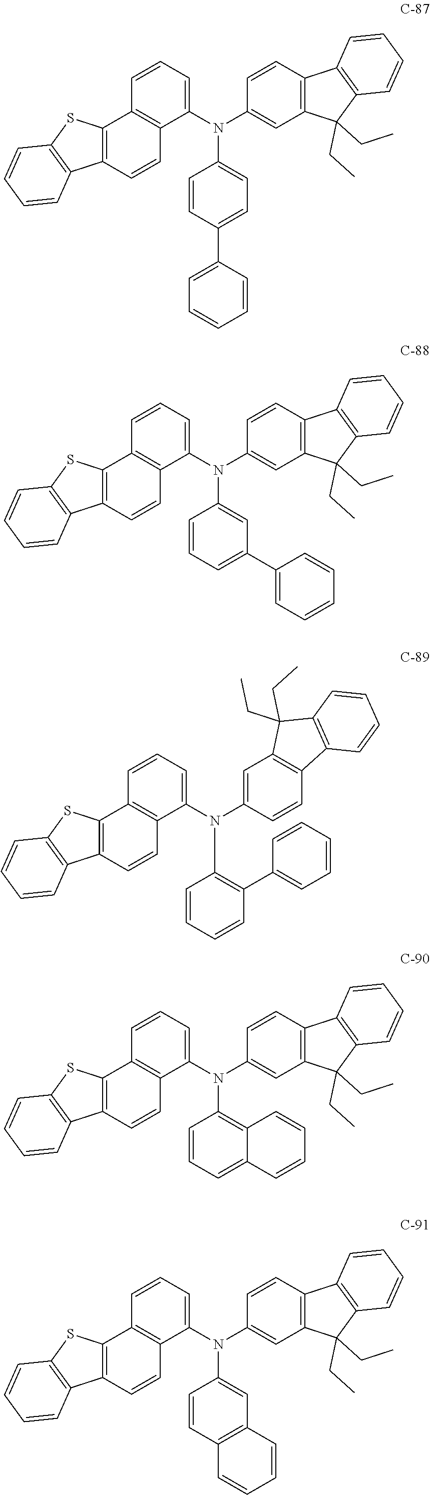 [SMILES: CCC1(CC)c2ccccc2-c2ccc(N(c3ccc(-c4ccccc4)cc3)c3cccc4c3ccc3c5ccccc5sc43)cc21 ; CCC1(CC)c2ccccc2-c2ccc(N(c3cccc(-c4ccccc4)c3)c3cccc4c3ccc3c5ccccc5sc43)cc21 ; CCC1(CC)c2ccccc2-c2ccc(N(c3ccccc3-c3ccccc3)c3cccc4c3ccc3c5ccccc5sc43)cc21 ; CCC1(CC)c2ccccc2-c2ccc(N(c3cccc4ccccc34)c3cccc4c3ccc3c5ccccc5sc43)cc21 ; CCC1(CC)c2ccccc2-c2ccc(N(c3ccc4ccccc4c3)c3cccc4c3ccc3c5ccccc5sc43)cc21]

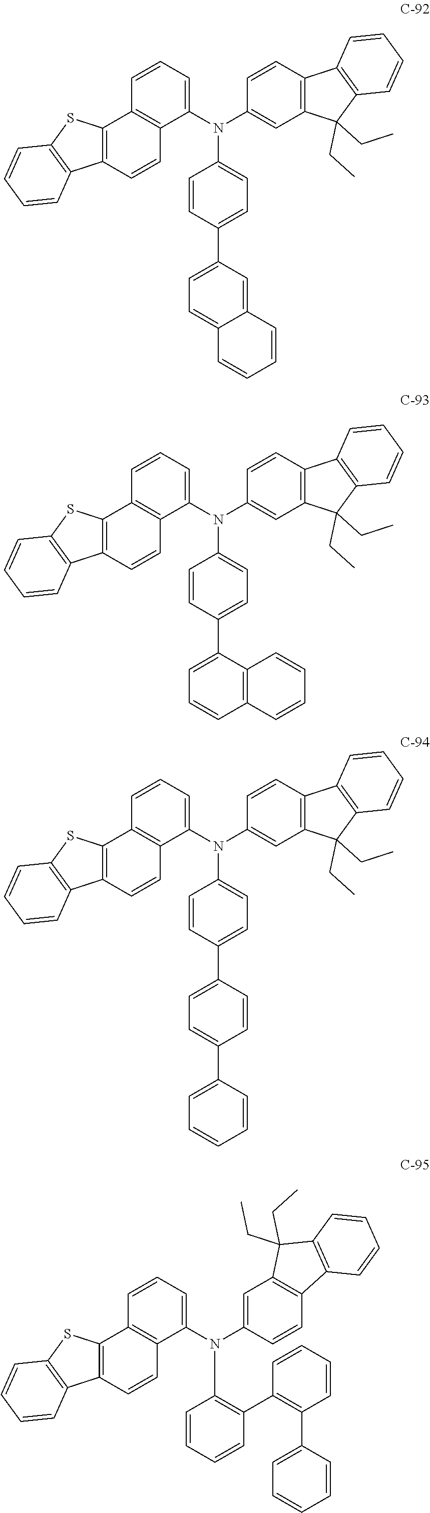 [SMILES: CCC1(CC)c2ccccc2-c2ccc(N(c3ccc(-c4ccc5ccccc5c4)cc3)c3cccc4c3ccc3c5ccccc5sc43)cc21 ; CCC1(CC)c2ccccc2-c2ccc(N(c3ccc(-c4cccc5ccccc45)cc3)c3cccc4c3ccc3c5ccccc5sc43)cc21 ; CCC1(CC)c2ccccc2-c2ccc(N(c3ccc(-c4ccc(-c5ccccc5)cc4)cc3)c3cccc4c3ccc3c5ccccc5sc43)cc21 ; CCC1(CC)c2ccccc2-c2ccc(N(c3ccccc3-c3ccccc3-c3ccccc3)c3cccc4c3ccc3c5ccccc5sc43)cc21]

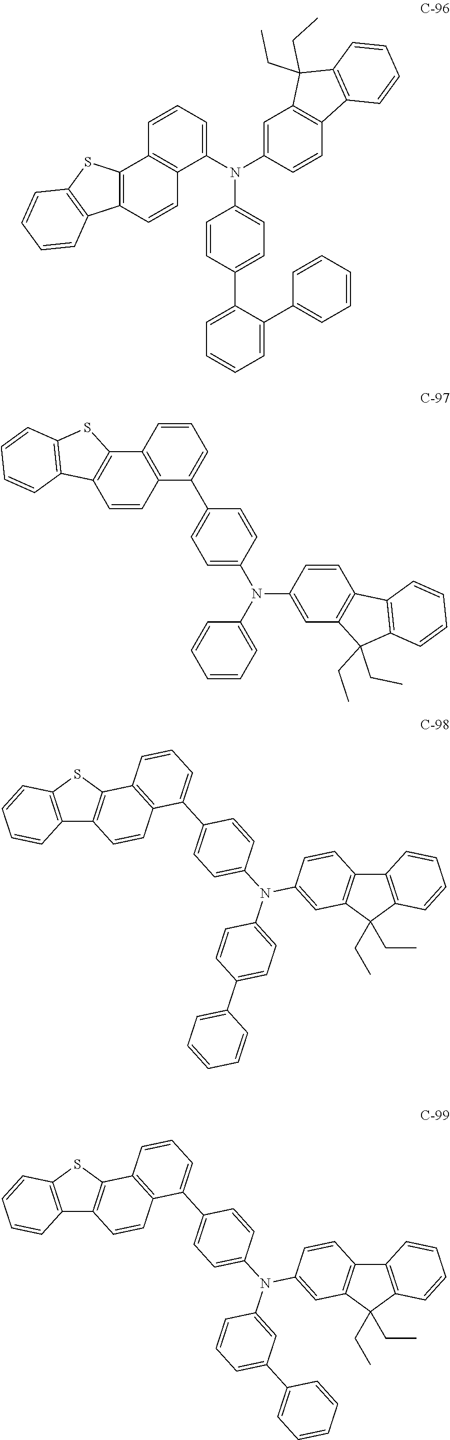 [SMILES: CCC1(CC)c2ccccc2-c2ccc(N(c3ccc(-c4ccccc4-c4ccccc4)cc3)c3cccc4c3ccc3c5ccccc5sc43)cc21 ; CCC1(CC)c2ccccc2-c2ccc(N(c3ccccc3)c3ccc(-c4cccc5c4ccc4c6ccccc6sc54)cc3)cc21 ; CCC1(CC)c2ccccc2-c2ccc(N(c3ccc(-c4ccccc4)cc3)c3ccc(-c4cccc5c4ccc4c6ccccc6sc54)cc3)cc21 ; CCC1(CC)c2ccccc2-c2ccc(N(c3ccc(-c4cccc5c4ccc4c6ccccc6sc54)cc3)c3cccc(-c4ccccc4)c3)cc21]

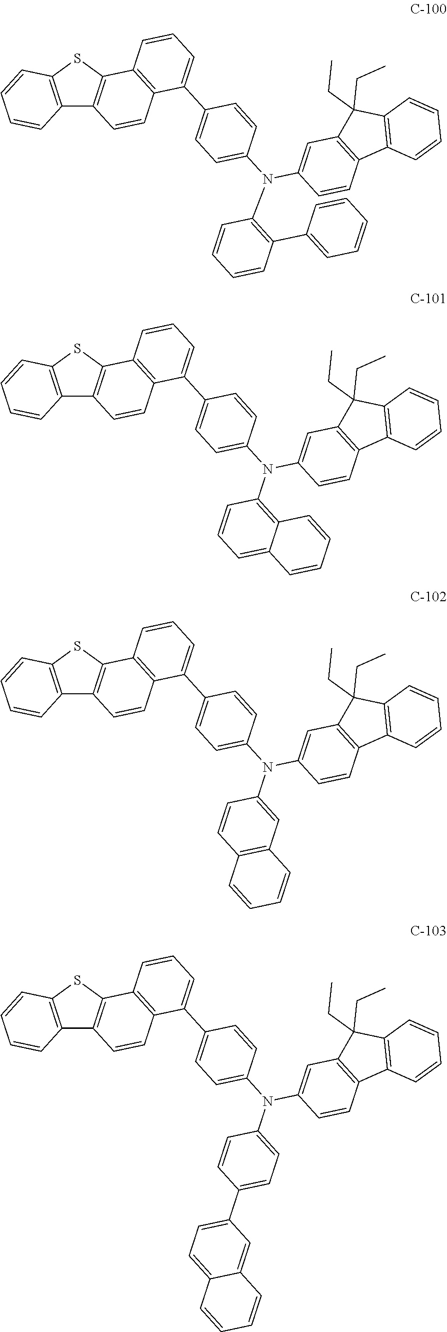 [SMILES: CCC1(CC)c2ccccc2-c2ccc(N(c3ccc(-c4cccc5c4ccc4c6ccccc6sc54)cc3)c3ccccc3-c3ccccc3)cc21 ; CCC1(CC)c2ccccc2-c2ccc(N(c3ccc(-c4cccc5c4ccc4c6ccccc6sc54)cc3)c3cccc4ccccc34)cc21 ; CCC1(CC)c2ccccc2-c2ccc(N(c3ccc(-c4cccc5c4ccc4c6ccccc6sc54)cc3)c3ccc4ccccc4c3)cc21 ; CCC1(CC)c2ccccc2-c2ccc(N(c3ccc(-c4ccc5ccccc5c4)cc3)c3ccc(-c4cccc5c4ccc4c6ccccc6sc54)cc3)cc21]

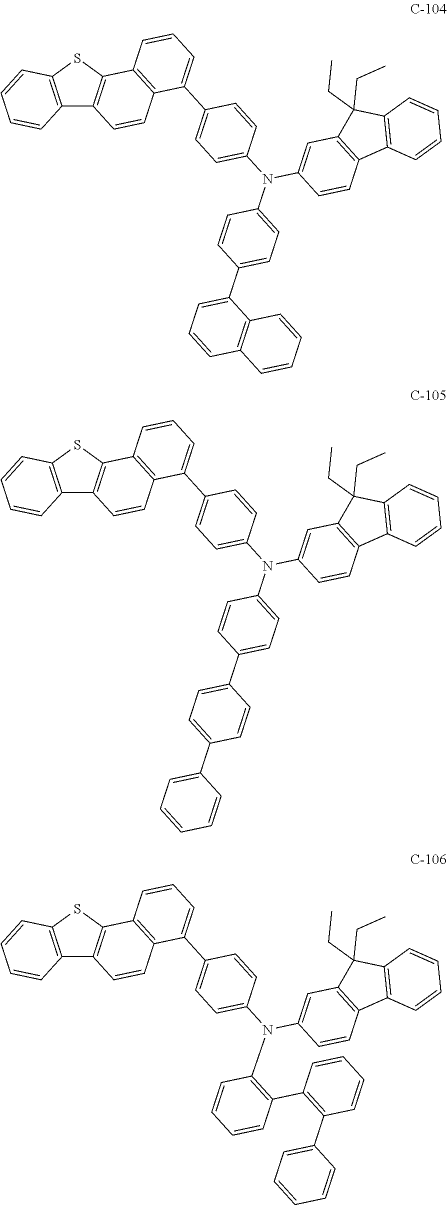 [SMILES: CCC1(CC)c2ccccc2-c2ccc(N(c3ccc(-c4cccc5ccccc45)cc3)c3ccc(-c4cccc5c4ccc4c6ccccc6sc54)cc3)cc21 ; CCC1(CC)c2ccccc2-c2ccc(N(c3ccc(-c4ccc(-c5ccccc5)cc4)cc3)c3ccc(-c4cccc5c4ccc4c6ccccc6sc54)cc3)cc21 ; CCC1(CC)c2ccccc2-c2ccc(N(c3ccc(-c4cccc5c4ccc4c6ccccc6sc54)cc3)c3ccccc3-c3ccccc3-c3ccccc3)cc21]

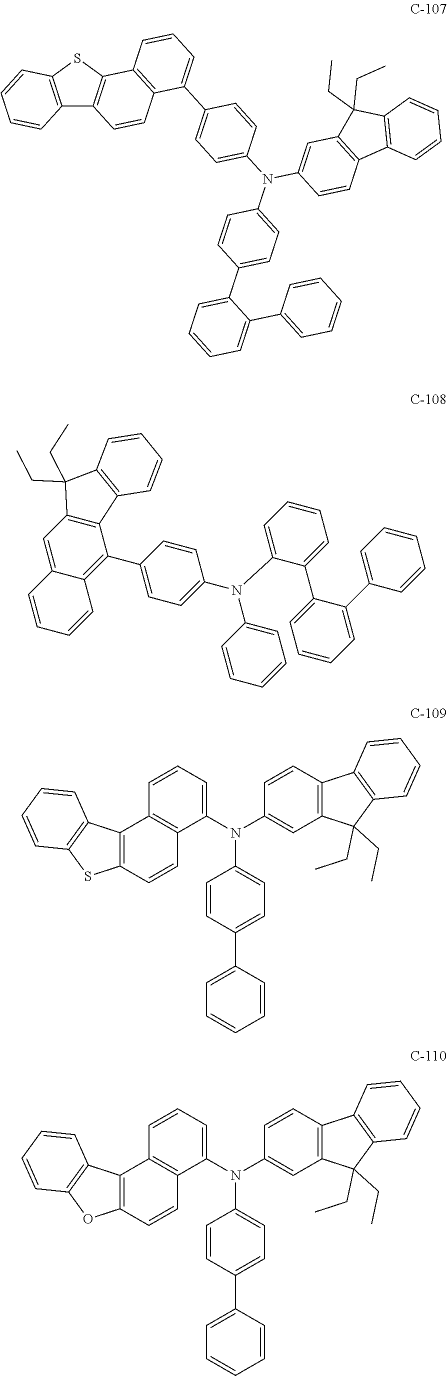 [SMILES: CCC1(CC)c2ccccc2-c2ccc(N(c3ccc(-c4ccccc4-c4ccccc4)cc3)c3ccc(-c4cccc5c4ccc4c6ccccc6sc54)cc3)cc21 ; CCC1(CC)c2ccccc2-c2c1cc1ccccc1c2-c1ccc(N(c2ccccc2)c2ccccc2-c2ccccc2-c2ccccc2)cc1 ; CCC1(CC)c2ccccc2-c2ccc(N(c3ccc(-c4ccccc4)cc3)c3cccc4c3ccc3sc5ccccc5c34)cc21 ; CCC1(CC)c2ccccc2-c2ccc(N(c3ccc(-c4ccccc4)cc3)c3cccc4c3ccc3oc5ccccc5c34)cc21]

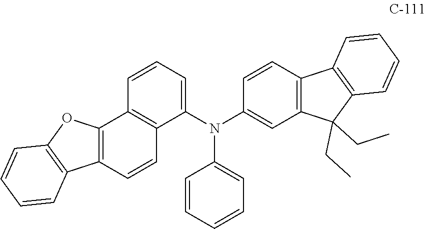 [SMILES: CCC1(CC)c2ccccc2-c2ccc(N(c3ccccc3)c3cccc4c3ccc3c5ccccc5oc43)cc21]

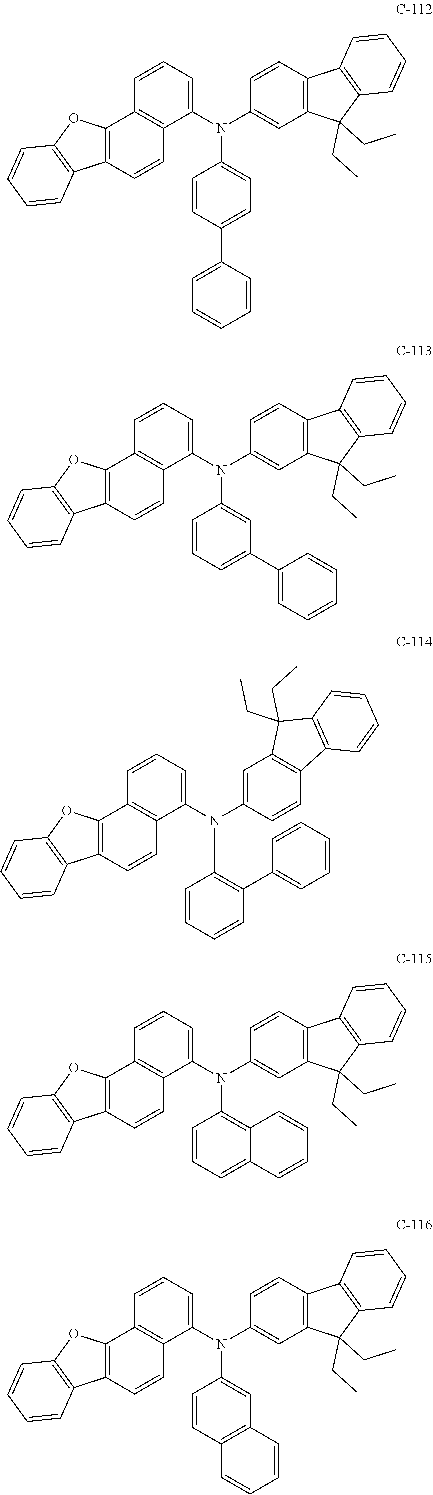 [SMILES: CCC1(CC)c2ccccc2-c2ccc(N(c3ccc(-c4ccccc4)cc3)c3cccc4c3ccc3c5ccccc5oc43)cc21 ; CCC1(CC)c2ccccc2-c2ccc(N(c3cccc(-c4ccccc4)c3)c3cccc4c3ccc3c5ccccc5oc43)cc21 ; CCC1(CC)c2ccccc2-c2ccc(N(c3ccccc3-c3ccccc3)c3cccc4c3ccc3c5ccccc5oc43)cc21 ; CCC1(CC)c2ccccc2-c2ccc(N(c3cccc4ccccc34)c3cccc4c3ccc3c5ccccc5oc43)cc21 ; CCC1(CC)c2ccccc2-c2ccc(N(c3ccc4ccccc4c3)c3cccc4c3ccc3c5ccccc5oc43)cc21]

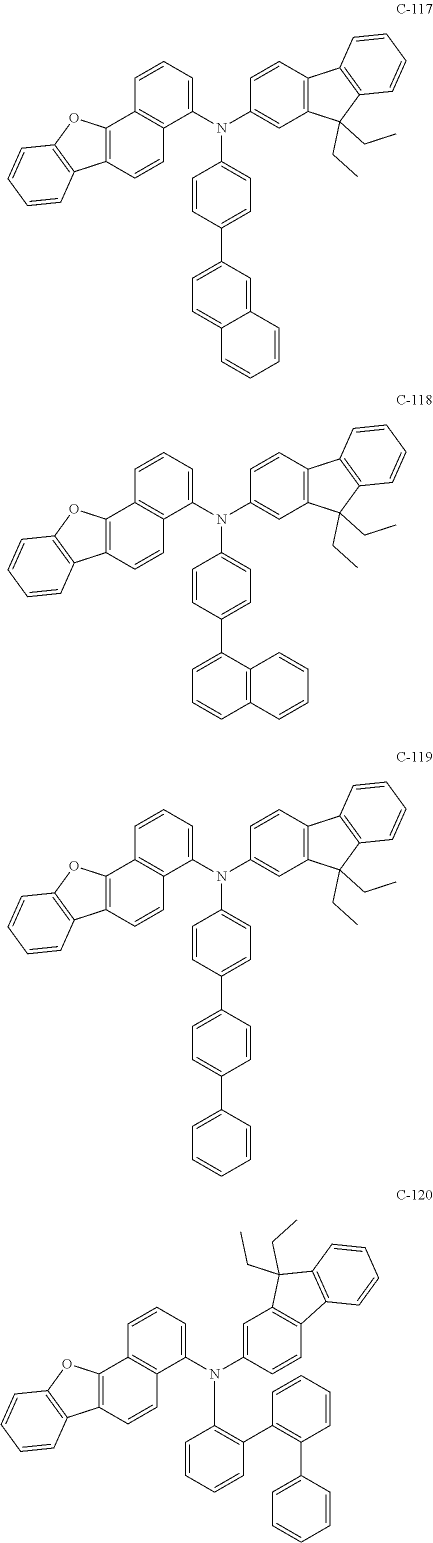 [SMILES: CCC1(CC)c2ccccc2-c2ccc(N(c3ccc(-c4ccc5ccccc5c4)cc3)c3cccc4c3ccc3c5ccccc5oc43)cc21 ; CCC1(CC)c2ccccc2-c2ccc(N(c3ccc(-c4cccc5ccccc45)cc3)c3cccc4c3ccc3c5ccccc5oc43)cc21 ; CCC1(CC)c2ccccc2-c2ccc(N(c3ccc(-c4ccc(-c5ccccc5)cc4)cc3)c3cccc4c3ccc3c5ccccc5oc43)cc21 ; CCC1(CC)c2ccccc2-c2ccc(N(c3ccccc3-c3ccccc3-c3ccccc3)c3cccc4c3ccc3c5ccccc5oc43)cc21]

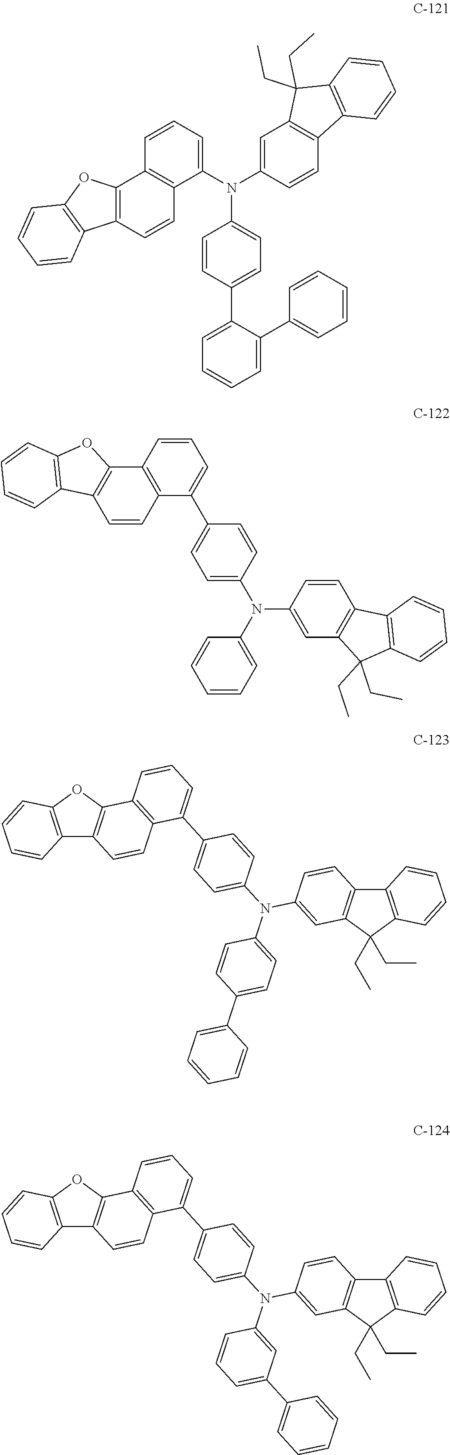 [SMILES: CCC1(CC)c2ccccc2-c2ccc(N(c3ccc(-c4ccccc4-c4ccccc4)cc3)c3cccc4c3ccc3c5ccccc5oc43)cc21 ; CCC1(CC)c2ccccc2-c2ccc(N(c3ccccc3)c3ccc(-c4cccc5c4ccc4c6ccccc6oc54)cc3)cc21 ; CCC1(CC)c2ccccc2-c2ccc(N(c3ccc(-c4ccccc4)cc3)c3ccc(-c4cccc5c4ccc4c6ccccc6oc54)cc3)cc21 ; CCC1(CC)c2ccccc2-c2ccc(N(c3ccc(-c4cccc5c4ccc4c6ccccc6oc54)cc3)c3cccc(-c4ccccc4)c3)cc21]

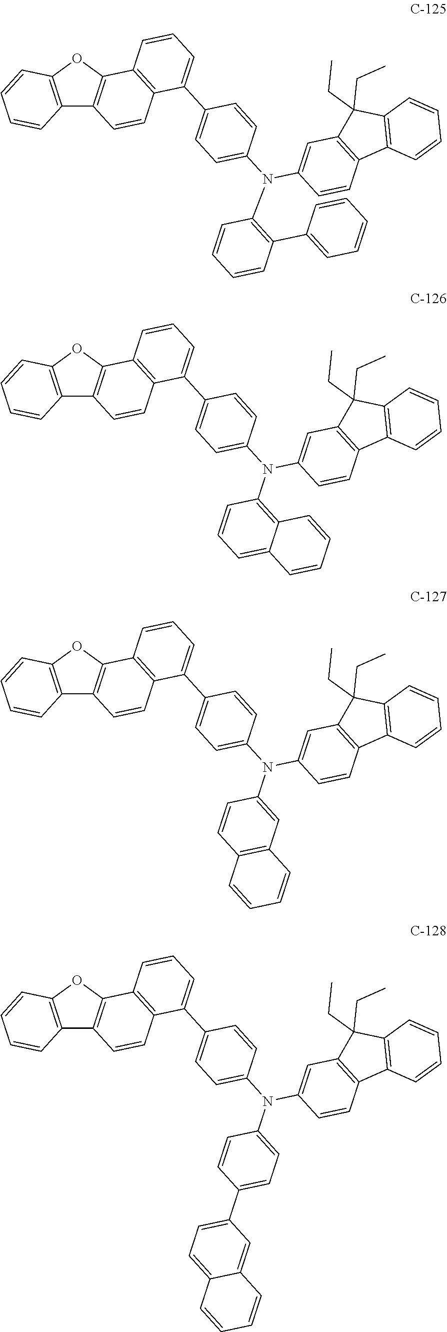 [SMILES: CCC1(CC)c2ccccc2-c2ccc(N(c3ccc(-c4cccc5c4ccc4c6ccccc6oc54)cc3)c3ccccc3-c3ccccc3)cc21 ; CCC1(CC)c2ccccc2-c2ccc(N(c3ccc(-c4cccc5c4ccc4c6ccccc6oc54)cc3)c3cccc4ccccc34)cc21 ; CCC1(CC)c2ccccc2-c2ccc(N(c3ccc(-c4cccc5c4ccc4c6ccccc6oc54)cc3)c3ccc4ccccc4c3)cc21 ; CCC1(CC)c2ccccc2-c2ccc(N(c3ccc(-c4ccc5ccccc5c4)cc3)c3ccc(-c4cccc5c4ccc4c6ccccc6oc54)cc3)cc21]

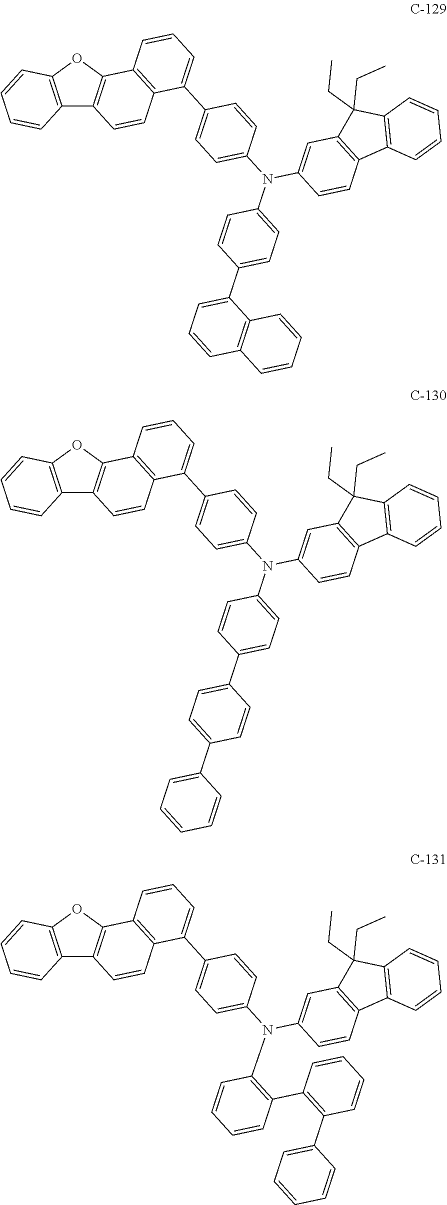 [SMILES: CCC1(CC)c2ccccc2-c2ccc(N(c3ccc(-c4cccc5ccccc45)cc3)c3ccc(-c4cccc5c4ccc4c6ccccc6oc54)cc3)cc21 ; CCC1(CC)c2ccccc2-c2ccc(N(c3ccc(-c4ccc(-c5ccccc5)cc4)cc3)c3ccc(-c4cccc5c4ccc4c6ccccc6oc54)cc3)cc21 ; CCC1(CC)c2ccccc2-c2ccc(N(c3ccc(-c4cccc5c4ccc4c6ccccc6oc54)cc3)c3ccccc3-c3ccccc3-c3ccccc3)cc21]

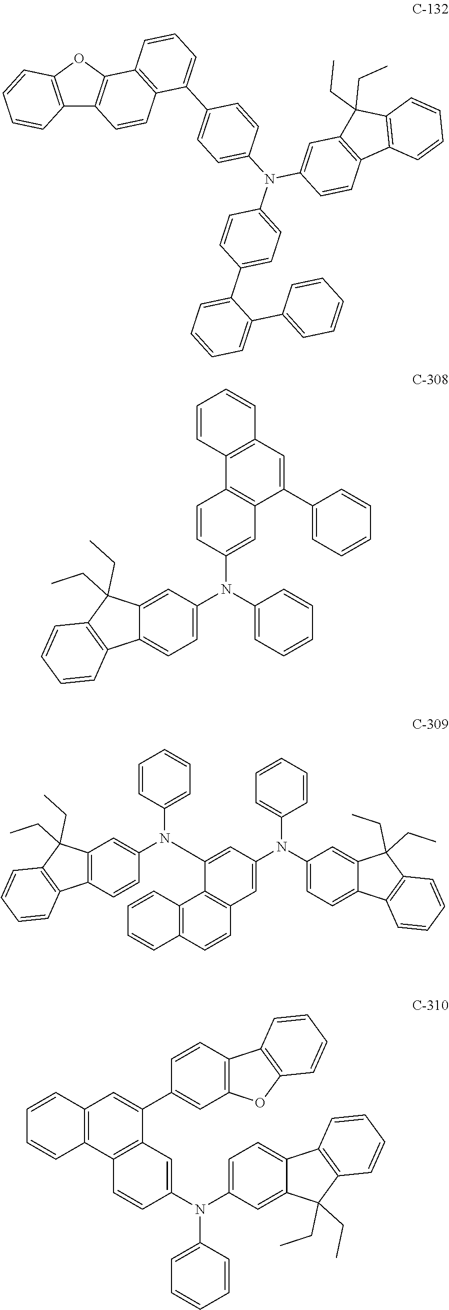

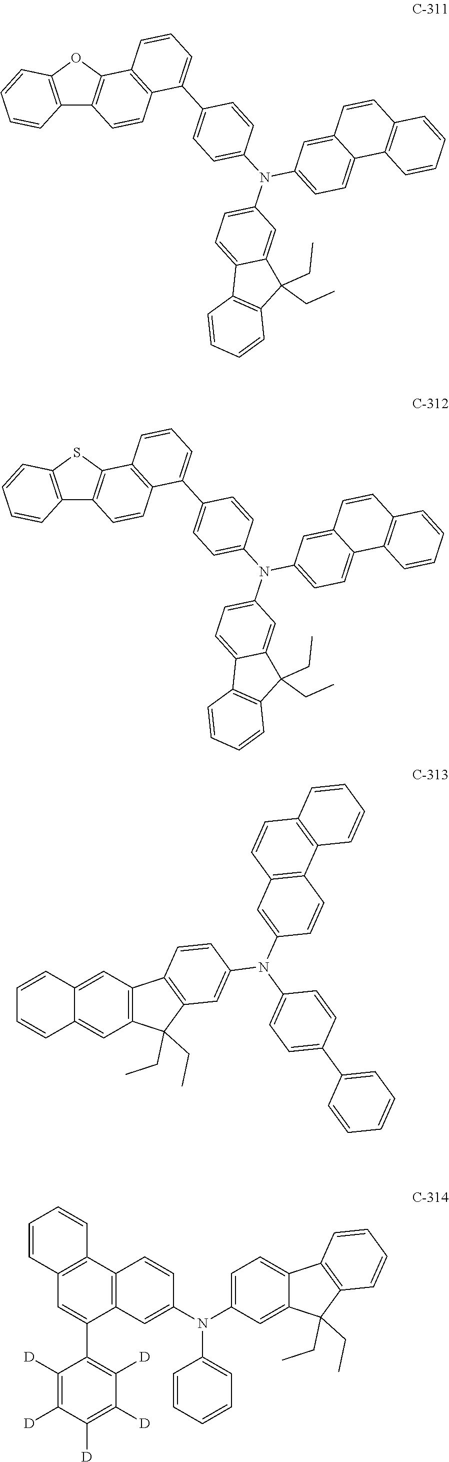 [SMILES: CCC1(CC)c2ccccc2-c2ccc(N(c3ccc(-c4cccc5c4ccc4c6ccccc6oc54)cc3)c3ccc4c(ccc5ccccc54)c3)cc21 ; CCC1(CC)c2ccccc2-c2ccc(N(c3ccc(-c4cccc5c4ccc4c6ccccc6sc54)cc3)c3ccc4c(ccc5ccccc54)c3)cc21 ; CCC1(CC)c2cc(N(c3ccc(-c4ccccc4)cc3)c3ccc4c(ccc5ccccc54)c3)ccc2-c2cc3ccccc3cc21 ; [2H]c1c([2H])c([2H])c(-c2cc3ccccc3c3ccc(N(c4ccccc4)c4ccc5c(c4)C(CC)(CC)c4ccccc4-5)cc23)c([2H])c1[2H]]

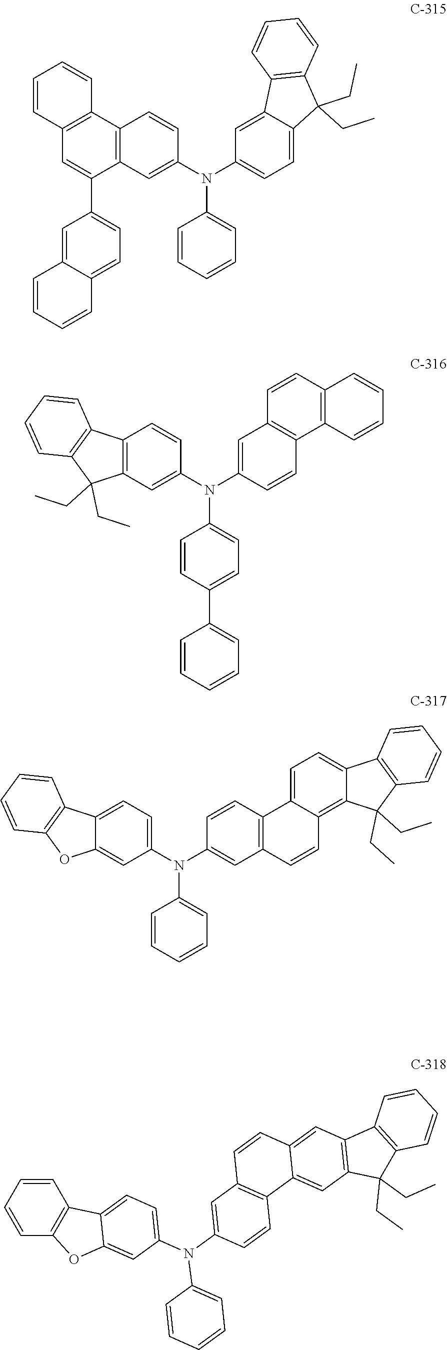 [SMILES: CCC1(CC)c2ccccc2-c2cc(N(c3ccccc3)c3ccc4c(c3)c(-c3ccc5ccccc5c3)cc3ccccc34)ccc21 ; CCC1(CC)c2ccccc2-c2ccc(N(c3ccc(-c4ccccc4)cc3)c3ccc4c(ccc5ccccc54)c3)cc21 ; CCC1(CC)c2ccccc2-c2ccc3c(ccc4cc(N(c5ccccc5)c5ccc6c(c5)oc5ccccc56)ccc43)c21 ; CCC1(CC)c2ccccc2-c2cc3ccc4cc(N(c5ccccc5)c5ccc6c(c5)oc5ccccc56)ccc4c3cc21]

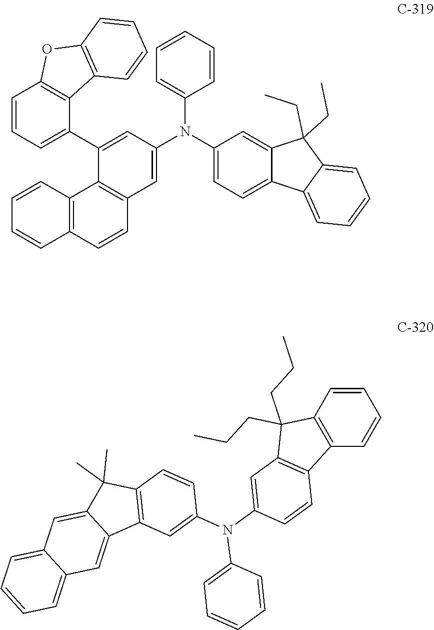 [SMILES: CCC1(CC)c2ccccc2-c2ccc(N(c3ccccc3)c3cc(-c4cccc5oc6ccccc6c45)c4c(ccc5ccccc54)c3)cc21 ; CCCC1(CCC)c2ccccc2-c2ccc(N(c3ccccc3)c3ccc4c(c3)-c3cc5ccccc5cc3C4(C)C)cc21]

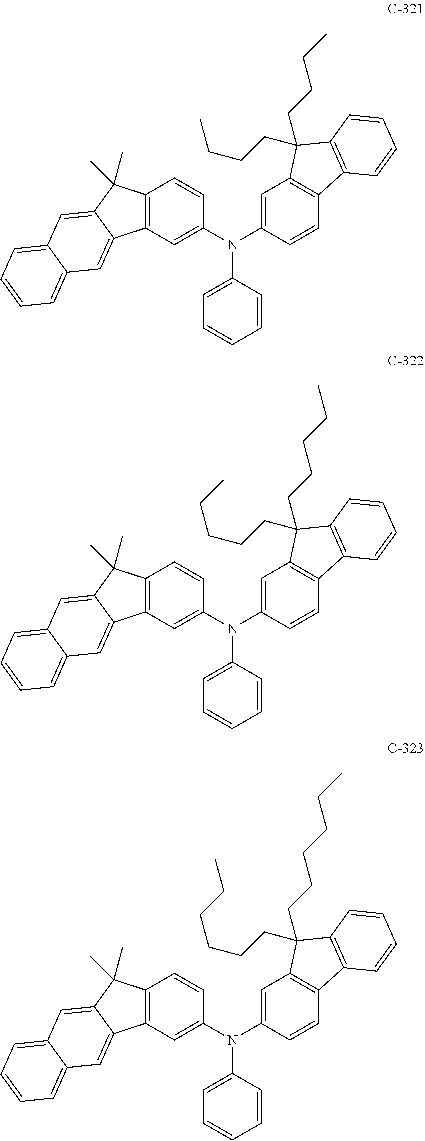 [SMILES: CCCCC1(CCCC)c2ccccc2-c2ccc(N(c3ccccc3)c3ccc4c(c3)-c3cc5ccccc5cc3C4(C)C)cc21 ; CCCCCC1(CCCCC)c2ccccc2-c2ccc(N(c3ccccc3)c3ccc4c(c3)-c3cc5ccccc5cc3C4(C)C)cc21 ; CCCCCCC1(CCCCCC)c2ccccc2-c2ccc(N(c3ccccc3)c3ccc4c(c3)-c3cc5ccccc5cc3C4(C)C)cc21]

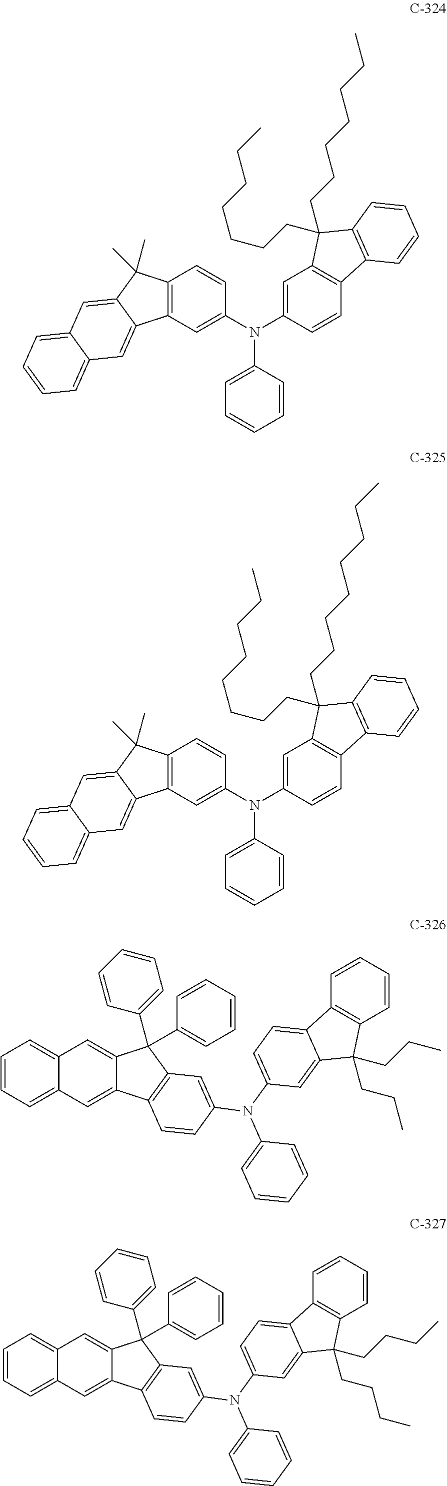 [SMILES: CCCCCCCC1(CCCCCCC)c2ccccc2-c2ccc(N(c3ccccc3)c3ccc4c(c3)-c3cc5ccccc5cc3C4(C)C)cc21 ; CCCCCCCCC1(CCCCCCCC)c2ccccc2-c2ccc(N(c3ccccc3)c3ccc4c(c3)-c3cc5ccccc5cc3C4(C)C)cc21 ; CCCC1(CCC)c2ccccc2-c2ccc(N(c3ccccc3)c3ccc4c(c3)C(c3ccccc3)(c3ccccc3)c3cc5ccccc5cc3-4)cc21 ; CCCCC1(CCCC)c2ccccc2-c2ccc(N(c3ccccc3)c3ccc4c(c3)C(c3ccccc3)(c3ccccc3)c3cc5ccccc5cc3-4)cc21]

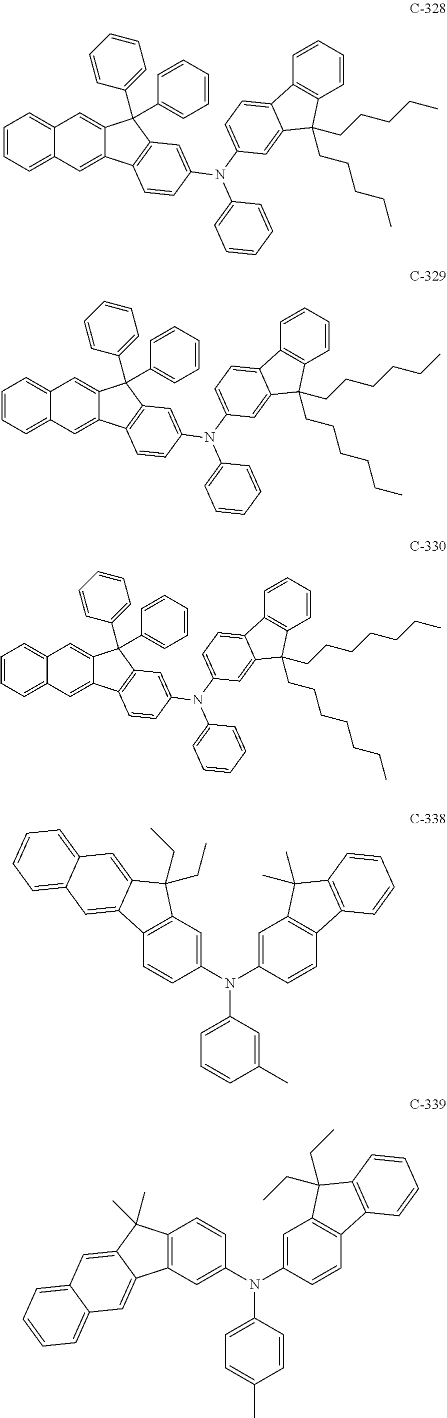 [SMILES: CCCCCC1(CCCCC)c2ccccc2-c2ccc(N(c3ccccc3)c3ccc4c(c3)C(c3ccccc3)(c3ccccc3)c3cc5ccccc5cc3-4)cc21 ; CCCCCCC1(CCCCCC)c2ccccc2-c2ccc(N(c3ccccc3)c3ccc4c(c3)C(c3ccccc3)(c3ccccc3)c3cc5ccccc5cc3-4)cc21 ; CCCCCCCC1(CCCCCCC)c2ccccc2-c2ccc(N(c3ccccc3)c3ccc4c(c3)C(c3ccccc3)(c3ccccc3)c3cc5ccccc5cc3-4)cc21 ; CCC1(CC)c2cc(N(c3cccc(C)c3)c3ccc4c(c3)C(C)(C)c3ccccc3-4)ccc2-c2cc3ccccc3cc21 ; CCC1(CC)c2ccccc2-c2ccc(N(c3ccc(C)cc3)c3ccc4c(c3)-c3cc5ccccc5cc3C4(C)C)cc21]

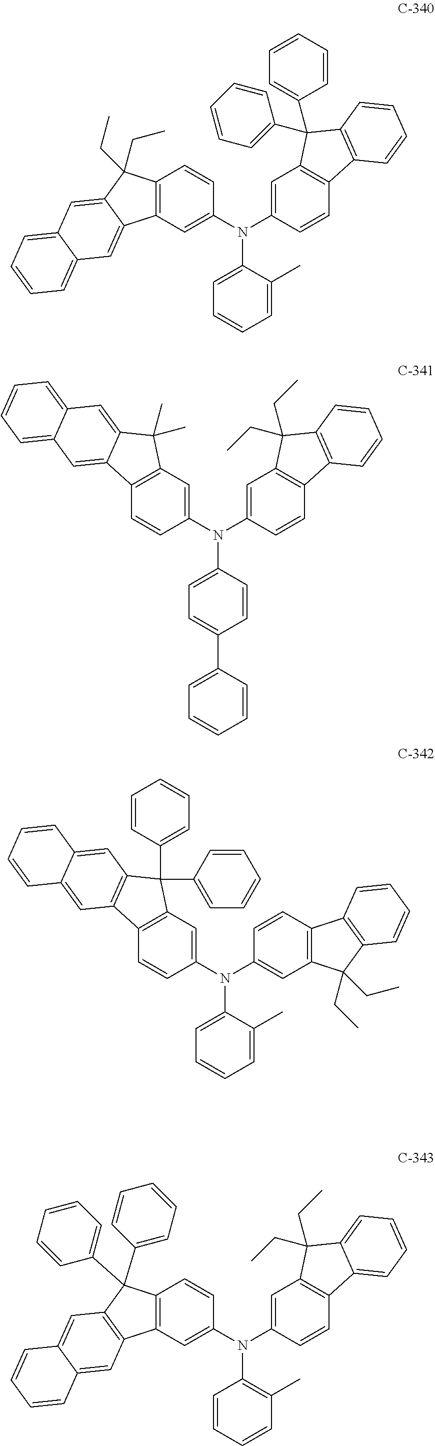 [SMILES: CCC1(CC)c2ccc(N(c3ccc4c(c3)C(c3ccccc3)(c3ccccc3)c3ccccc3-4)c3ccccc3C)cc2-c2cc3ccccc3cc21 ; CCC1(CC)c2ccccc2-c2ccc(N(c3ccc(-c4ccccc4)cc3)c3ccc4c(c3)C(C)(C)c3cc5ccccc5cc3-4)cc21 ; CCC1(CC)c2ccccc2-c2ccc(N(c3ccc4c(c3)C(c3ccccc3)(c3ccccc3)c3cc5ccccc5cc3-4)c3ccccc3C)cc21 ; CCC1(CC)c2ccccc2-c2ccc(N(c3ccc4c(c3)-c3cc5ccccc5cc3C4(c3ccccc3)c3ccccc3)c3ccccc3C)cc21]

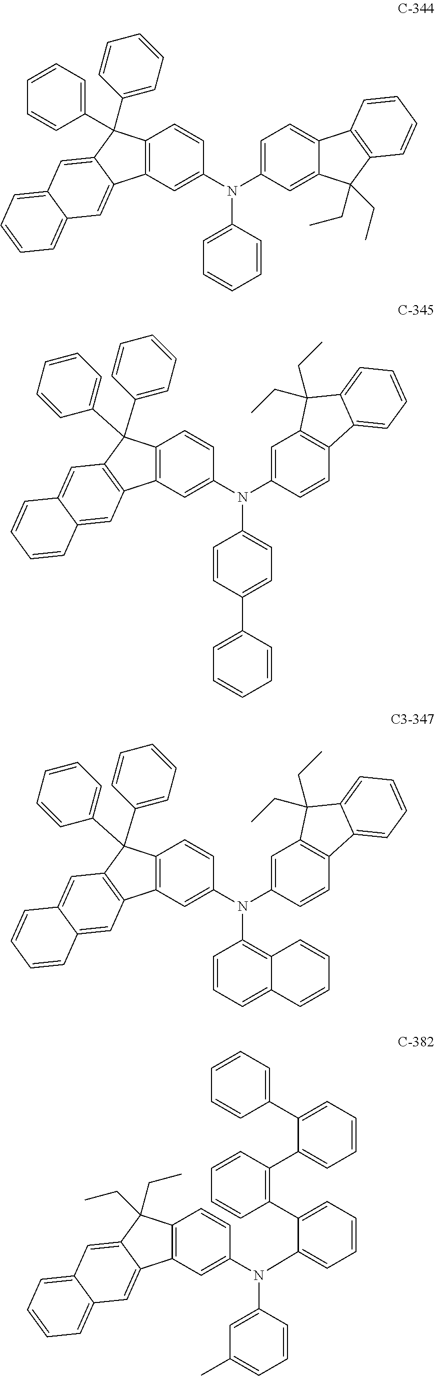

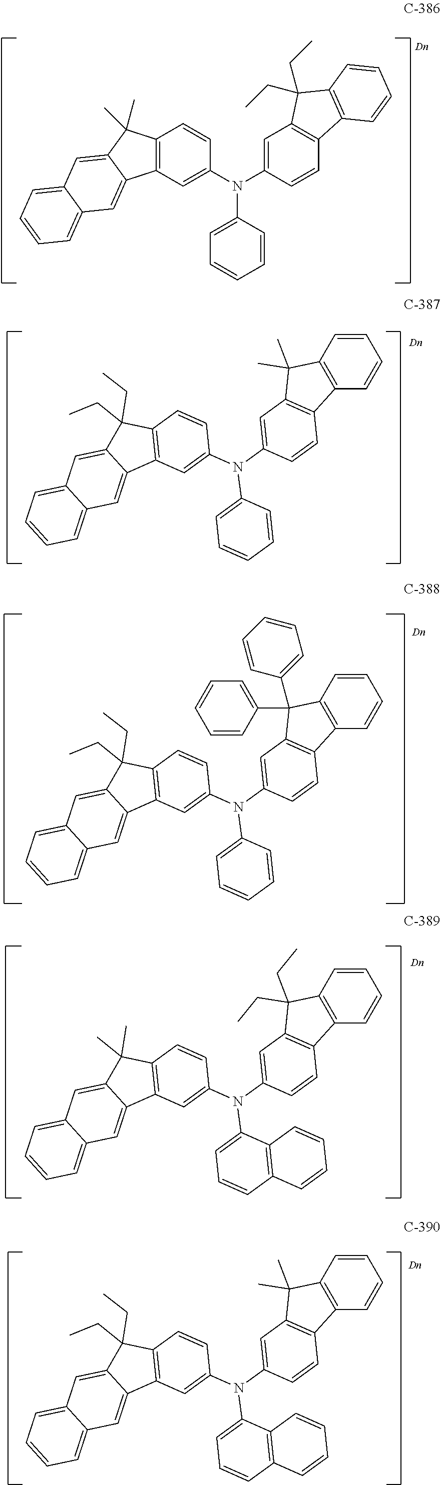

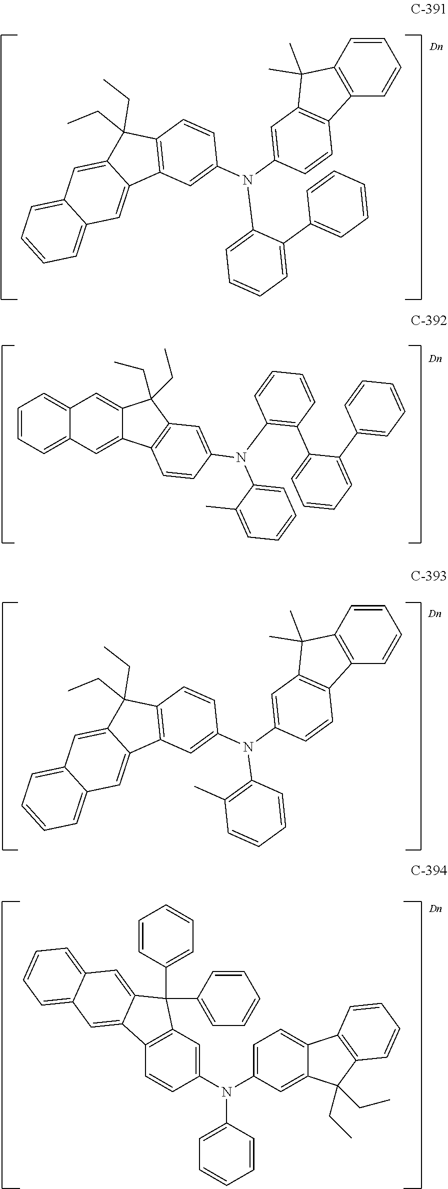

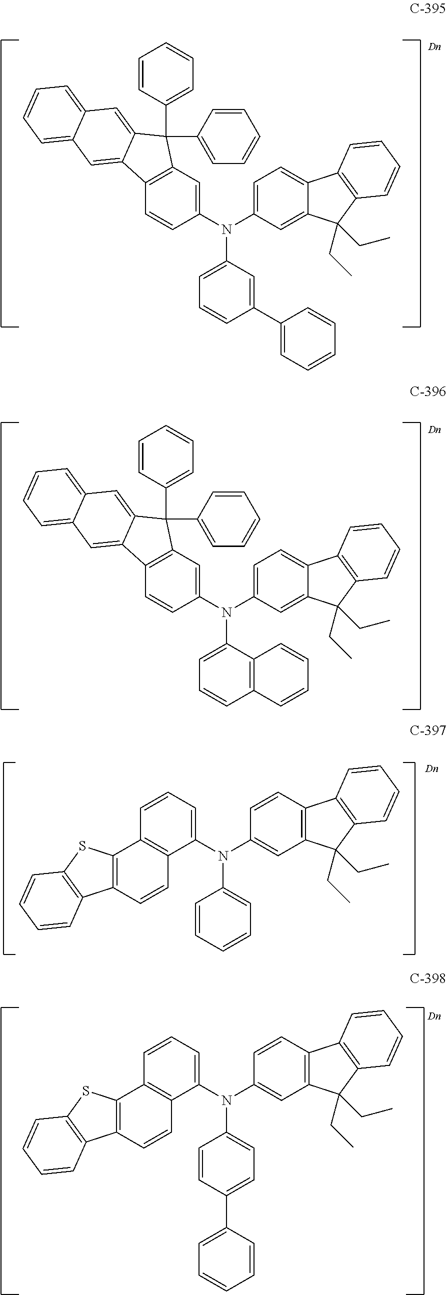

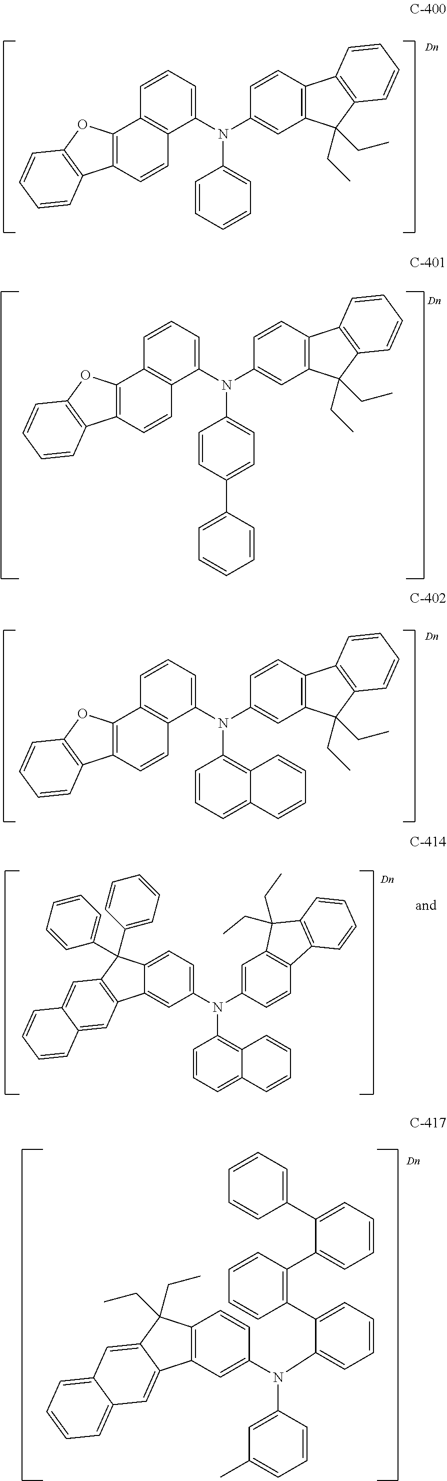

Wherein in the compounds above, Dn represents that n number of hydrogens are replaced with deuterium, and n represents an integer of 1 or more, which is an integer of 1 to the maximum number of hydrogens in the compound.

The organic electroluminescent device according to one embodiment of the present disclosure, may comprise the organic electroluminescent compound represented by Formula 1.

In addition, the present disclosure provides the organic electroluminescent compound represented by the following Formula 3.

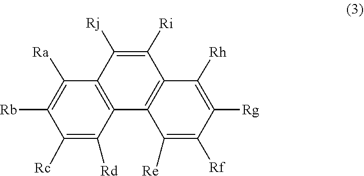

In Formula 3,
at least one of Rₐ to Rⱼ is represented by the following Formula 3-1,

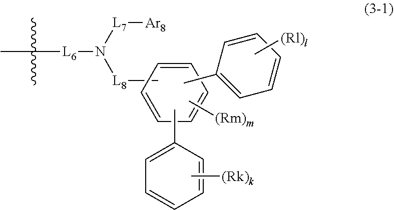

In Formula 3 and Formula 3-1,

Rₐ to Rₘ each independently represent hydrogen, deuterium, a halogen, a cyano, a substituted or unsubstituted (C1-C30)alkyl, a substituted or unsubstituted (C3-C30)cycloalkyl, a substituted or unsubstituted (C6-C30)aryl, or a substituted or unsubstituted (3- to 30-membered)heteroaryl. According to one embodiment of the present disclosure, Rₐ to Rₘ each independently may represent hydrogen, deuterium, a substituted or unsubstituted (C6-C20)aryl, or a substituted or unsubstituted (3- to 20-membered)heteroaryl. For example, Rₐ to Rₘ each independently may represent hydrogen, deuterium, a phenyl substituted or unsubstituted with deuterium, a naphthyl, a biphenyl substituted or unsubstituted with deuterium, or a m-terphenyl substituted or unsubstituted with deuterium.

[symbol] represents a bond between Formula 3 and Formula 3-1.

L₆ to L₈ each independently represent a single bond, a substituted or unsubstituted (C6-C30)arylene, or a substituted or unsubstituted (3- to 30-membered)heteroarylene. According to one embodiment of the present disclosure, L₆ to L₈ each independently may represent a single bond, or a substituted or unsubstituted (C6-C20)arylene. For example, L₆ to L₈ each independently may represent a single bond, a phenylene substituted or unsubstituted with deuterium, a naphthylene, a biphenylene substituted or unsubstituted with deuterium, or a m-terphenylene substituted or unsubstituted with deuterium.

Ar₈ represents a substituted or unsubstituted (C6-C30)aryl, a substituted or unsubstituted (C3-C30)cycloalkyl, or a substituted or unsubstituted (3- to 30-membered)heteroaryl. According to one embodiment of the present disclosure, Ar₈ may represent a substituted or unsubstituted (C6-C20)aryl, a substituted or unsubstituted (C3-C20)cycloalkyl, or a substituted or unsubstituted (3- to 20-membered)heteroaryl. For example, Ar₈ may represent a phenyl substituted or unsubstituted with deuterium or a naphthyl, a biphenyl substituted or unsubstituted with deuterium, a naphthyl, a dibenzofuranyl, a dibenzothiophenyl, a dimethylfluorenyl, a diphenylfluorenyl, a dibenzoselenophenyl, a spirobifluorenyl, an o-terphenyl, a m-terphenyl, or an adamantyl.

k and l each independently represent an integer of 1 to 5, and m represents an integer of 1 to 3. For example, k and l each independently may represent an integer of 5, and m may represent an integer of 3.

If k to m represent an integer of 2 or more, each of Rₖ to Rₘ may be the same as or different from each other.

According to one embodiment of the present disclosure, the compound represented by Formula 3-1 is represented by any one of the following Formulas 3-1a to 3-1f.

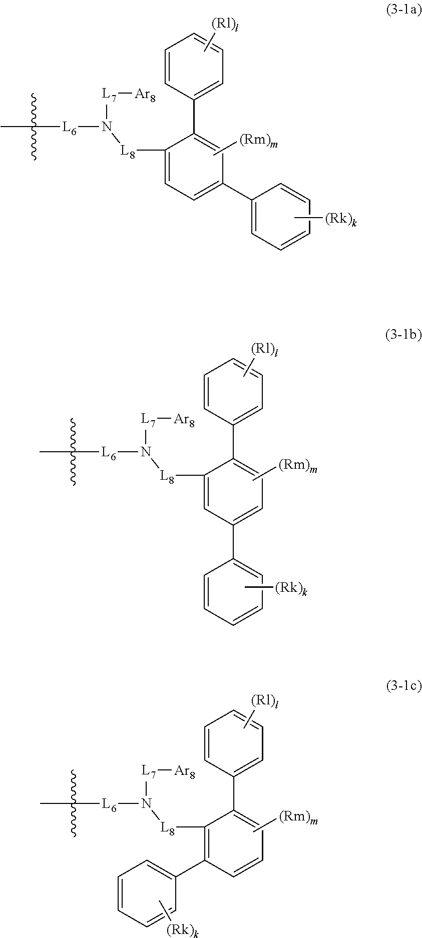

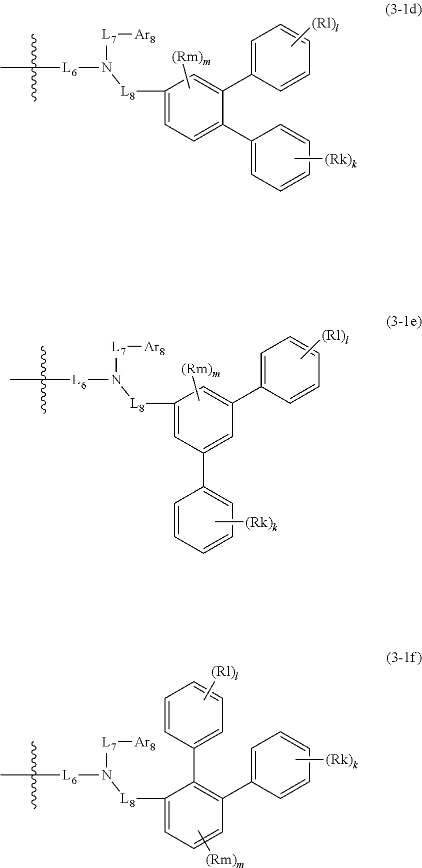

In Formulas 3-1a to 3-1f,
[symbol] represents a linking group connected to Formula 3;
L₆ to L₈, Ar₈, and Rₖ to Rₘ are as defined in Formula 3 and Formula 3-1;
k and l each independently represent an integer of 1 to 5, and m represents an integer of 1 to 3;
and if k to m represent an integer of 2 or more, each of Rₖ to Rₘ may be the same as or different from each other.

The compound represented by Formula 3 may be at least one selected from the following compounds, but is not limited thereto.

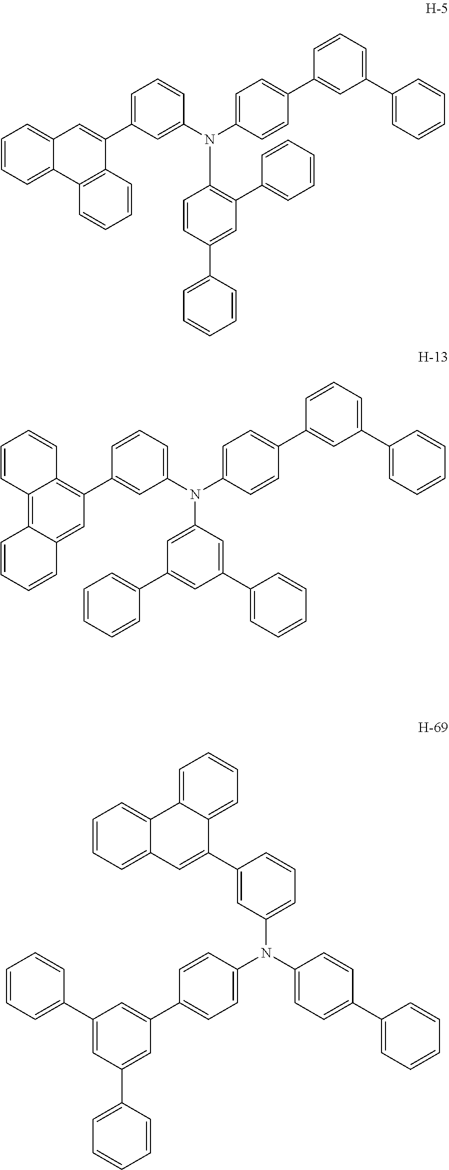 [SMILES: c1ccc(-c2cccc(-c3ccc(N(c4cccc(-c5cc6ccccc6c6ccccc56)c4)c4ccc(-c5ccccc5)cc4-c4ccccc4)cc3)c2)cc1 ; c1ccc(-c2cccc(-c3ccc(N(c4cc(-c5ccccc5)cc(-c5ccccc5)c4)c4cccc(-c5cc6ccccc6c6ccccc56)c4)cc3)c2)cc1 ; c1ccc(-c2ccc(N(c3ccc(-c4cc(-c5ccccc5)cc(-c5ccccc5)c4)cc3)c3cccc(-c4cc5ccccc5c5ccccc45)c3)cc2)cc1]

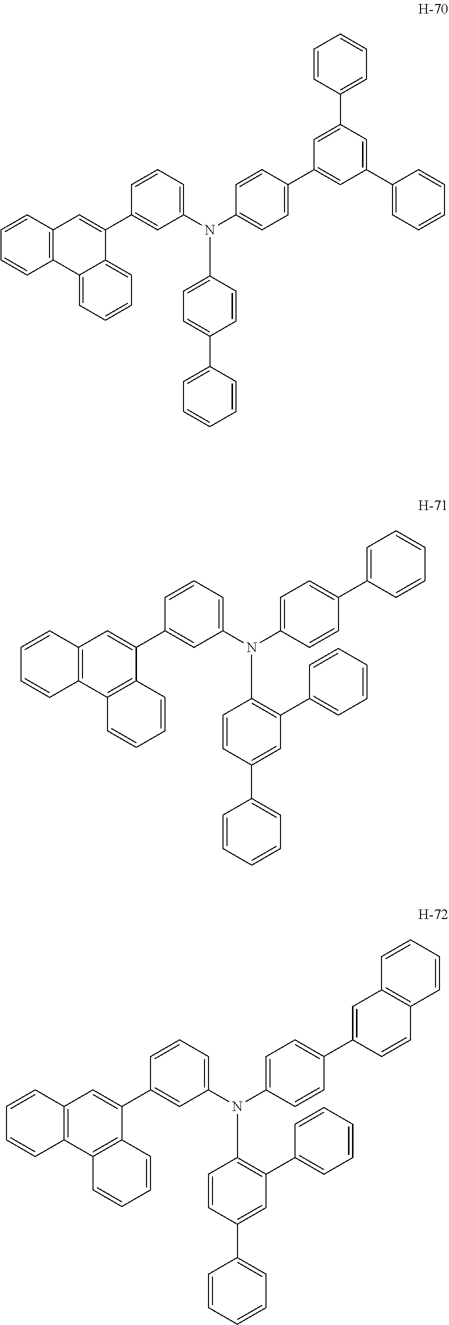 [SMILES: c1ccc(-c2ccc(N(c3ccc(-c4cc(-c5ccccc5)cc(-c5ccccc5)c4)cc3)c3cccc(-c4cc5ccccc5c5ccccc45)c3)cc2)cc1 ; c1ccc(-c2ccc(N(c3cccc(-c4cc5ccccc5c5ccccc45)c3)c3ccc(-c4ccccc4)cc3-c3ccccc3)cc2)cc1 ; c1ccc(-c2ccc(N(c3ccc(-c4ccc5ccccc5c4)cc3)c3cccc(-c4cc5ccccc5c5ccccc45)c3)c(-c3ccccc3)c2)cc1]

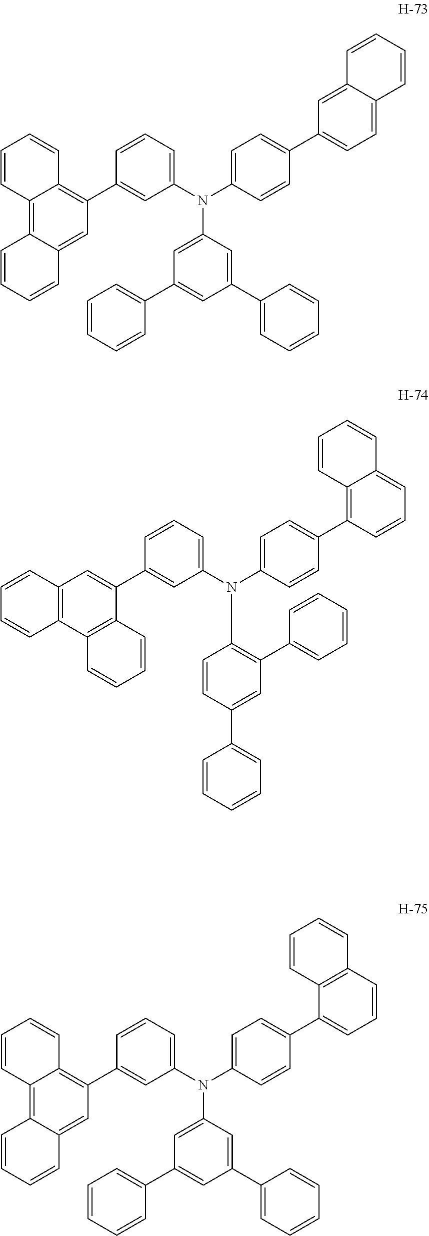 [SMILES: c1ccc(-c2cc(-c3ccccc3)cc(N(c3ccc(-c4ccc5ccccc5c4)cc3)c3cccc(-c4cc5ccccc5c5ccccc45)c3)c2)cc1 ; c1ccc(-c2ccc(N(c3ccc(-c4cccc5ccccc45)cc3)c3cccc(-c4cc5ccccc5c5ccccc45)c3)c(-c3ccccc3)c2)cc1 ; c1ccc(-c2cc(-c3ccccc3)cc(N(c3ccc(-c4cccc5ccccc45)cc3)c3cccc(-c4cc5ccccc5c5ccccc45)c3)c2)cc1]

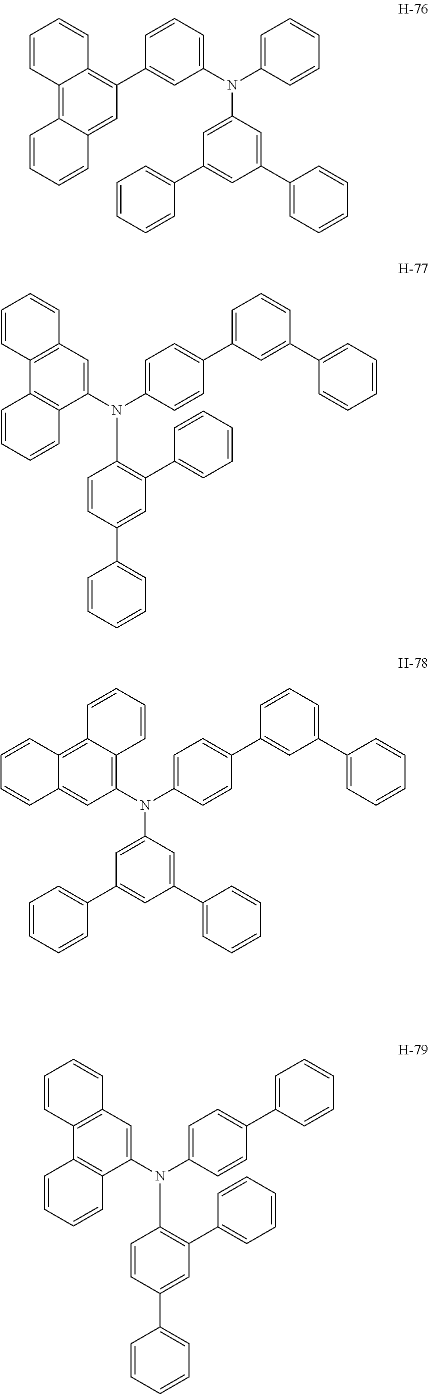 [SMILES: c1ccc(-c2cc(-c3ccccc3)cc(N(c3ccccc3)c3cccc(-c4cc5ccccc5c5ccccc45)c3)c2)cc1 ; c1ccc(-c2cccc(-c3ccc(N(c4ccc(-c5ccccc5)cc4-c4ccccc4)c4cc5ccccc5c5ccccc45)cc3)c2)cc1 ; c1ccc(-c2cccc(-c3ccc(N(c4cc(-c5ccccc5)cc(-c5ccccc5)c4)c4cc5ccccc5c5ccccc45)cc3)c2)cc1 ; c1ccc(-c2ccc(N(c3ccc(-c4ccccc4)cc3-c3ccccc3)c3cc4ccccc4c4ccccc34)cc2)cc1]

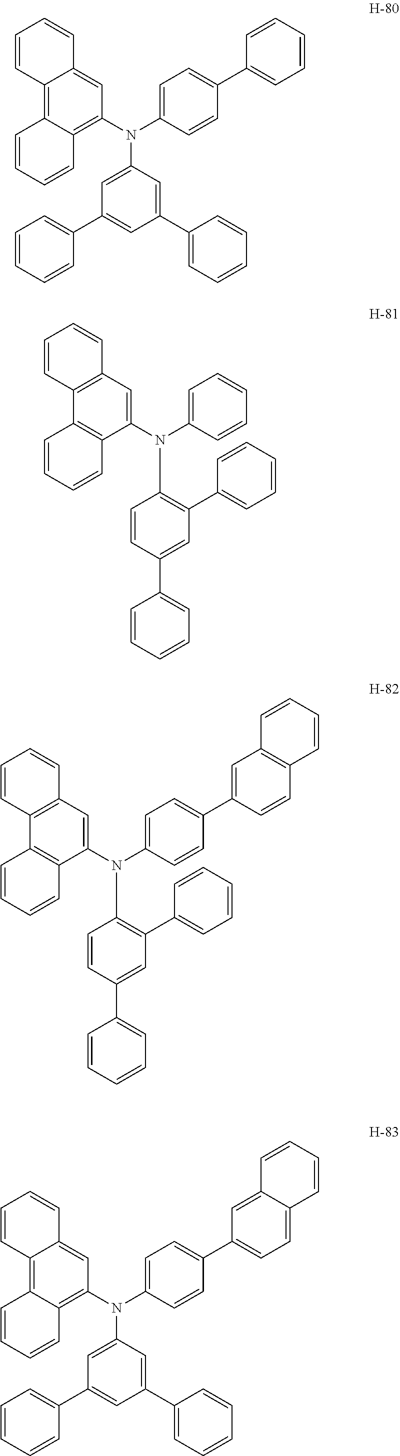 [SMILES: c1ccc(-c2ccc(N(c3cc(-c4ccccc4)cc(-c4ccccc4)c3)c3cc4ccccc4c4ccccc34)cc2)cc1 ; c1ccc(-c2ccc(N(c3ccccc3)c3cc4ccccc4c4ccccc34)c(-c3ccccc3)c2)cc1 ; c1ccc(-c2ccc(N(c3ccc(-c4ccc5ccccc5c4)cc3)c3cc4ccccc4c4ccccc34)c(-c3ccccc3)c2)cc1 ; c1ccc(-c2cc(-c3ccccc3)cc(N(c3ccc(-c4ccc5ccccc5c4)cc3)c3cc4ccccc4c4ccccc34)c2)cc1]

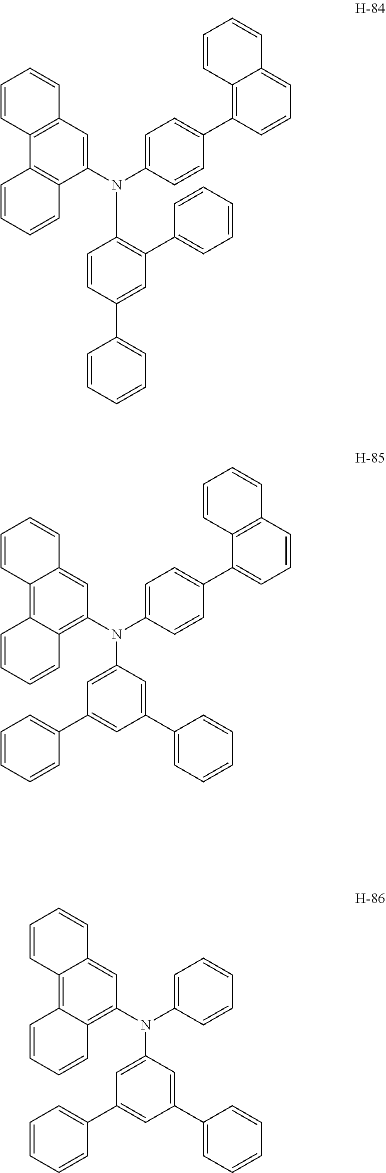 [SMILES: c1ccc(-c2ccc(N(c3ccc(-c4cccc5ccccc45)cc3)c3cc4ccccc4c4ccccc34)c(-c3ccccc3)c2)cc1 ; c1ccc(-c2cc(-c3ccccc3)cc(N(c3ccc(-c4cccc5ccccc45)cc3)c3cc4ccccc4c4ccccc34)c2)cc1 ; c1ccc(-c2cc(-c3ccccc3)cc(N(c3ccccc3)c3cc4ccccc4c4ccccc34)c2)cc1]

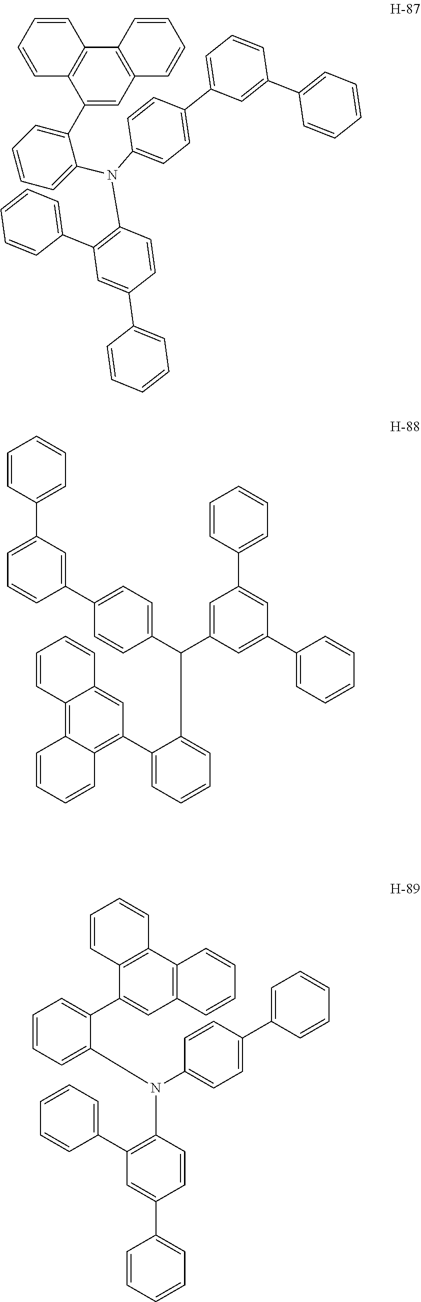

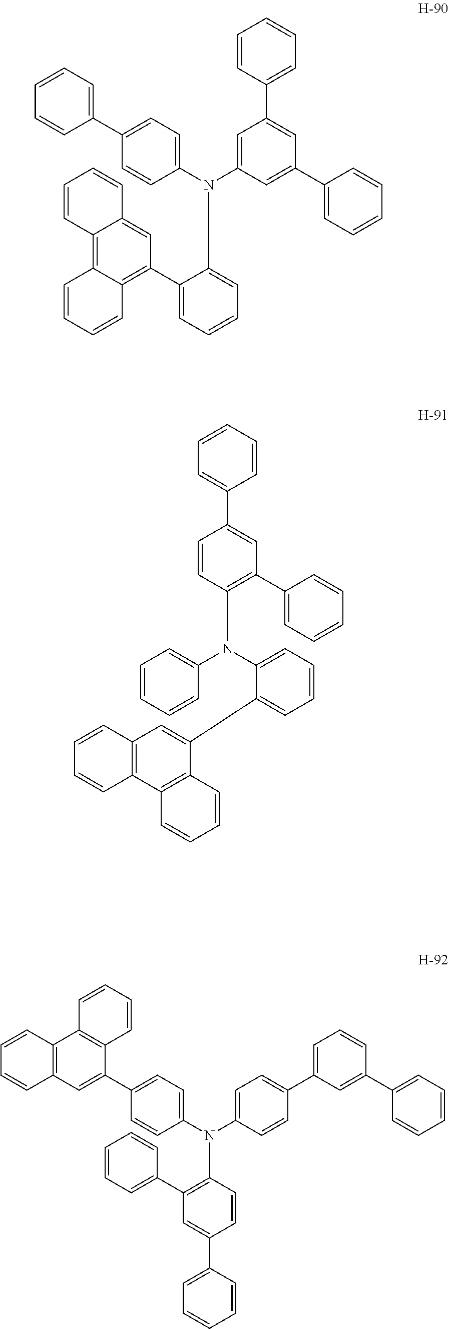 [SMILES: c1ccc(-c2ccc(N(c3cc(-c4ccccc4)cc(-c4ccccc4)c3)c3ccccc3-c3cc4ccccc4c4ccccc34)cc2)cc1 ; c1ccc(-c2ccc(N(c3ccccc3)c3ccccc3-c3cc4ccccc4c4ccccc34)c(-c3ccccc3)c2)cc1 ; c1ccc(-c2cccc(-c3ccc(N(c4ccc(-c5cc6ccccc6c6ccccc56)cc4)c4ccc(-c5ccccc5)cc4-c4ccccc4)cc3)c2)cc1]

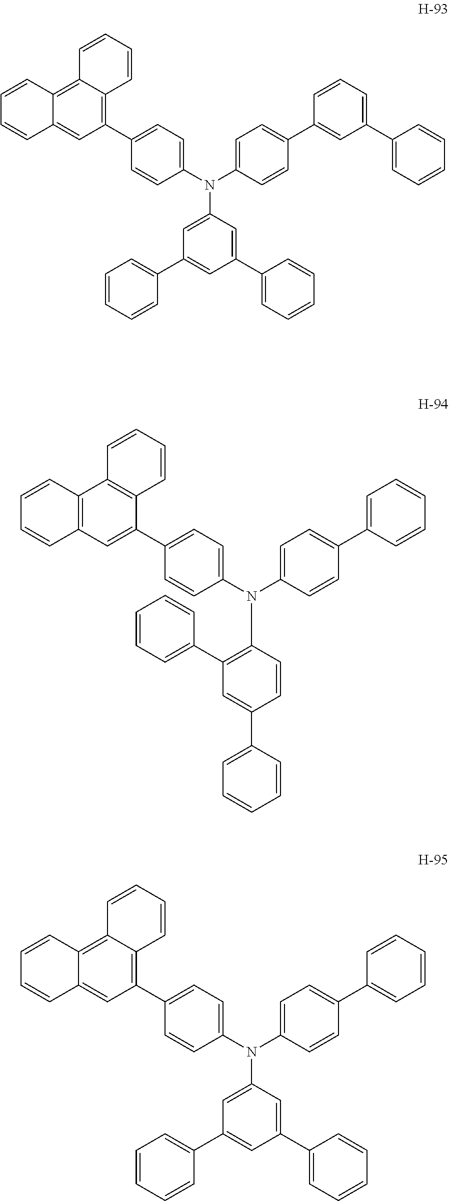 [SMILES: c1ccc(-c2cccc(-c3ccc(N(c4ccc(-c5cc6ccccc6c6ccccc56)cc4)c4cc(-c5ccccc5)cc(-c5ccccc5)c4)cc3)c2)cc1 ; c1ccc(-c2ccc(N(c3ccc(-c4cc5ccccc5c5ccccc45)cc3)c3ccc(-c4ccccc4)cc3-c3ccccc3)cc2)cc1 ; c1ccc(-c2ccc(N(c3ccc(-c4cc5ccccc5c5ccccc45)cc3)c3cc(-c4ccccc4)cc(-c4ccccc4)c3)cc2)cc1]

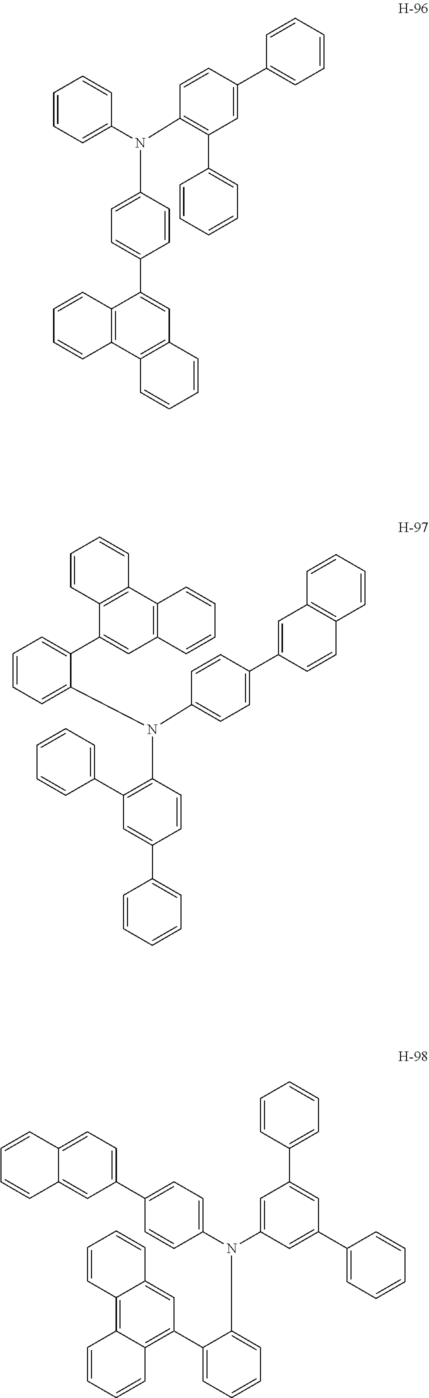 [SMILES: c1ccc(-c2ccc(N(c3ccccc3)c3ccc(-c4cc5ccccc5c5ccccc45)cc3)c(-c3ccccc3)c2)cc1 ; c1ccc(-c2ccc(N(c3ccc(-c4ccc5ccccc5c4)cc3)c3ccccc3-c3cc4ccccc4c4ccccc34)c(-c3ccccc3)c2)cc1 ; c1ccc(-c2cc(-c3ccccc3)cc(N(c3ccc(-c4ccc5ccccc5c4)cc3)c3ccccc3-c3cc4ccccc4c4ccccc34)c2)cc1]

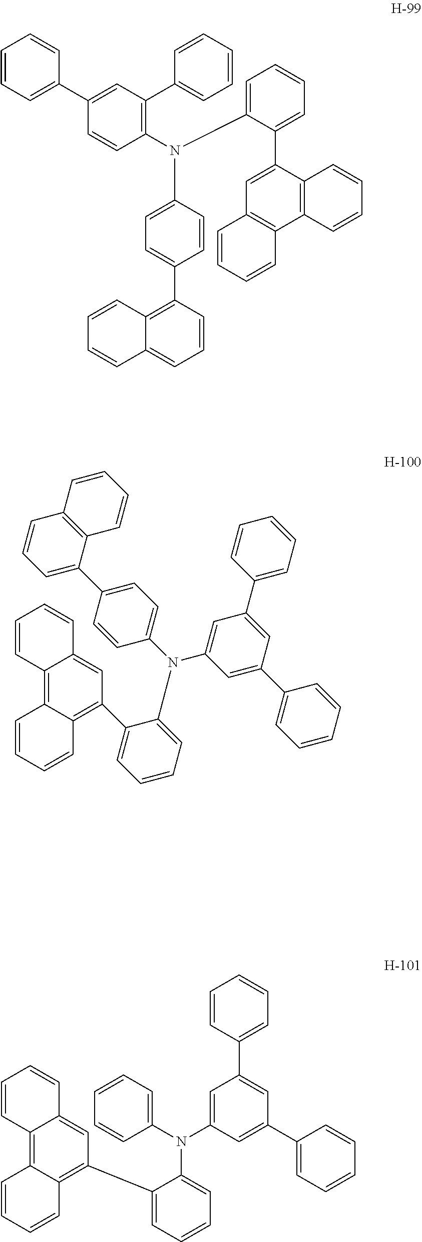 [SMILES: c1ccc(-c2ccc(N(c3ccc(-c4cccc5ccccc45)cc3)c3ccccc3-c3cc4ccccc4c4ccccc34)c(-c3ccccc3)c2)cc1 ; c1ccc(-c2cc(-c3ccccc3)cc(N(c3ccc(-c4cccc5ccccc45)cc3)c3ccccc3-c3cc4ccccc4c4ccccc34)c2)cc1 ; c1ccc(-c2cc(-c3ccccc3)cc(N(c3ccccc3)c3ccccc3-c3cc4ccccc4c4ccccc34)c2)cc1]

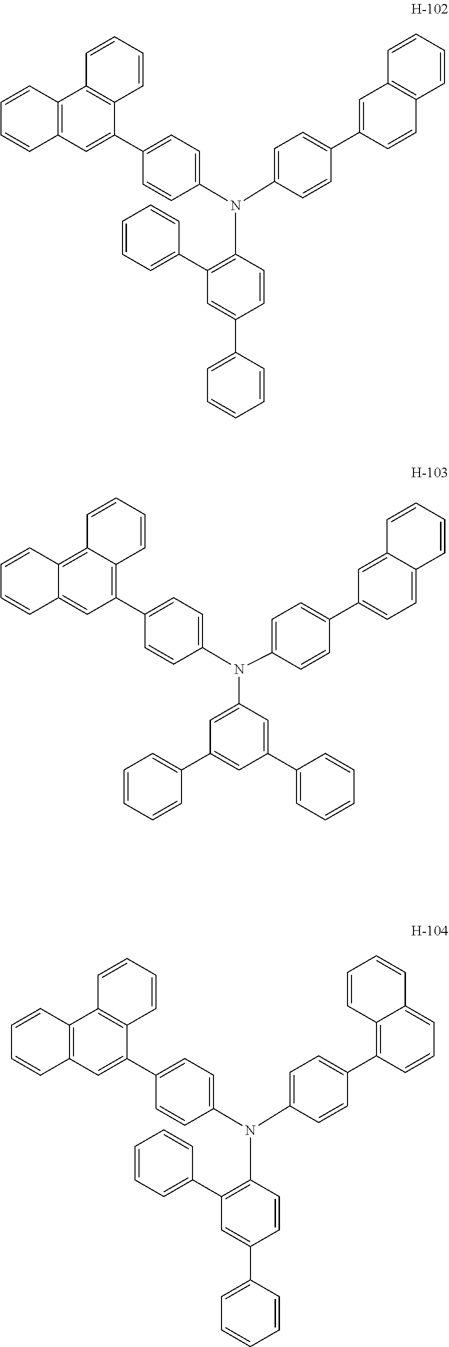 [SMILES: c1ccc(-c2ccc(N(c3ccc(-c4ccc5ccccc5c4)cc3)c3ccc(-c4cc5ccccc5c5ccccc45)cc3)c(-c3ccccc3)c2)cc1 ; c1ccc(-c2cc(-c3ccccc3)cc(N(c3ccc(-c4ccc5ccccc5c4)cc3)c3ccc(-c4cc5ccccc5c5ccccc45)cc3)c2)cc1 ; c1ccc(-c2ccc(N(c3ccc(-c4cccc5ccccc45)cc3)c3ccc(-c4cc5ccccc5c5ccccc45)cc3)c(-c3ccccc3)c2)cc1]

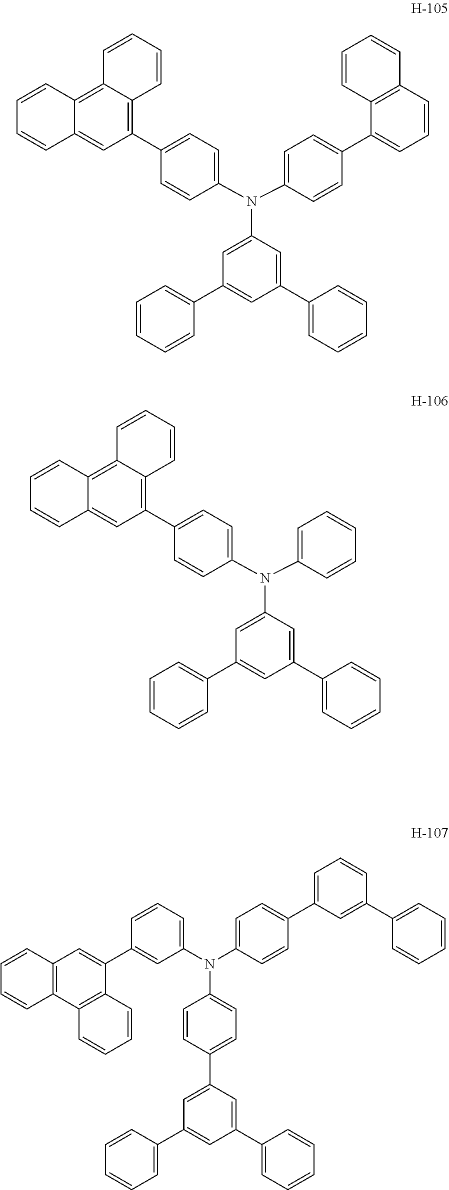 [SMILES: c1ccc(-c2cc(-c3ccccc3)cc(N(c3ccc(-c4cccc5ccccc45)cc3)c3ccc(-c4cc5ccccc5c5ccccc45)cc3)c2)cc1 ; c1ccc(-c2cc(-c3ccccc3)cc(N(c3ccccc3)c3ccc(-c4cc5ccccc5c5ccccc45)cc3)c2)cc1 ; c1ccc(-c2cccc(-c3ccc(N(c4ccc(-c5cc(-c6ccccc6)cc(-c6ccccc6)c5)cc4)c4cccc(-c5cc6ccccc6c6ccccc56)c4)cc3)c2)cc1]

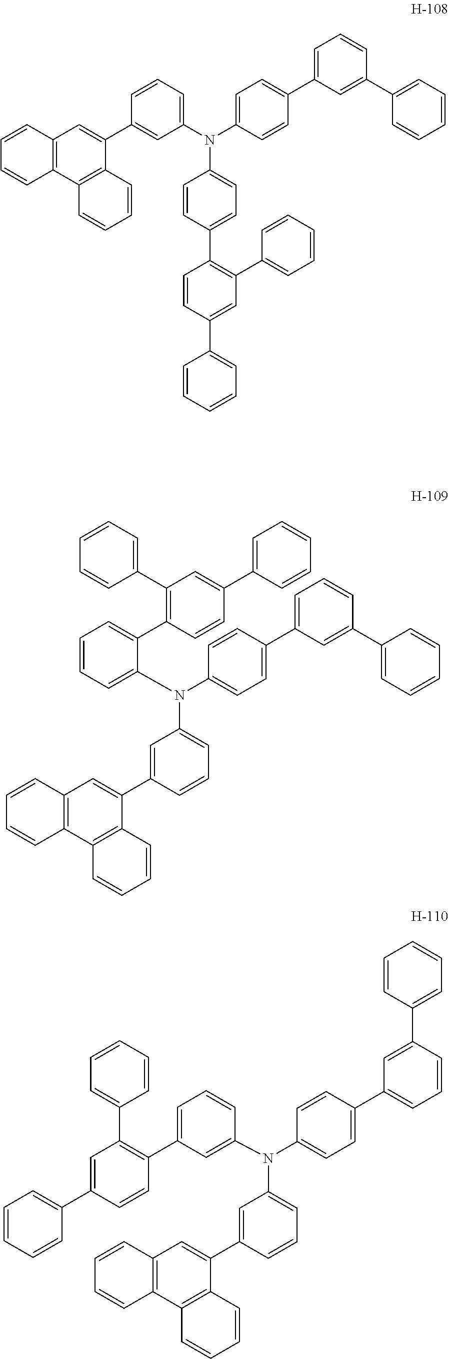 [SMILES: c1ccc(-c2cccc(-c3ccc(N(c4ccc(-c5ccc(-c6ccccc6)cc5-c5ccccc5)cc4)c4cccc(-c5cc6ccccc6c6ccccc56)c4)cc3)c2)cc1 ; c1ccc(-c2cccc(-c3ccc(N(c4cccc(-c5cc6ccccc6c6ccccc56)c4)c4ccccc4-c4ccc(-c5ccccc5)cc4-c4ccccc4)cc3)c2)cc1 ; c1ccc(-c2cccc(-c3ccc(N(c4cccc(-c5ccc(-c6ccccc6)cc5-c5ccccc5)c4)c4cccc(-c5cc6ccccc6c6ccccc56)c4)cc3)c2)cc1]

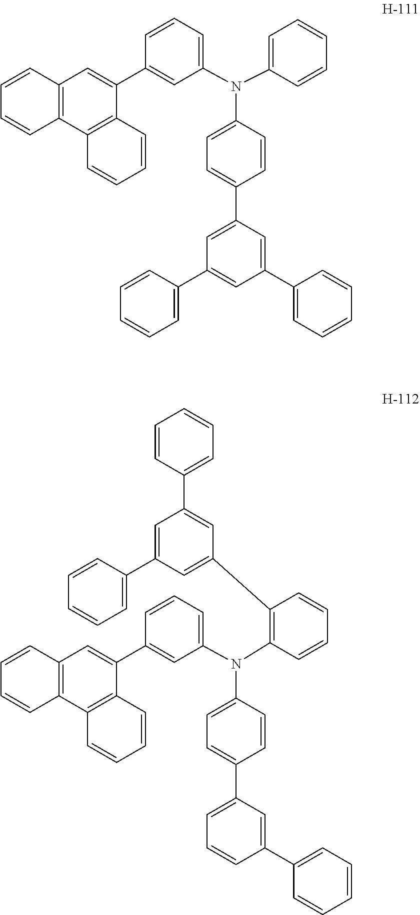 [SMILES: c1ccc(-c2cc(-c3ccccc3)cc(-c3ccc(N(c4ccccc4)c4cccc(-c5cc6ccccc6c6ccccc56)c4)cc3)c2)cc1 ; c1ccc(-c2cccc(-c3ccc(N(c4cccc(-c5cc6ccccc6c6ccccc56)c4)c4ccccc4-c4cc(-c5ccccc5)cc(-c5ccccc5)c4)cc3)c2)cc1]

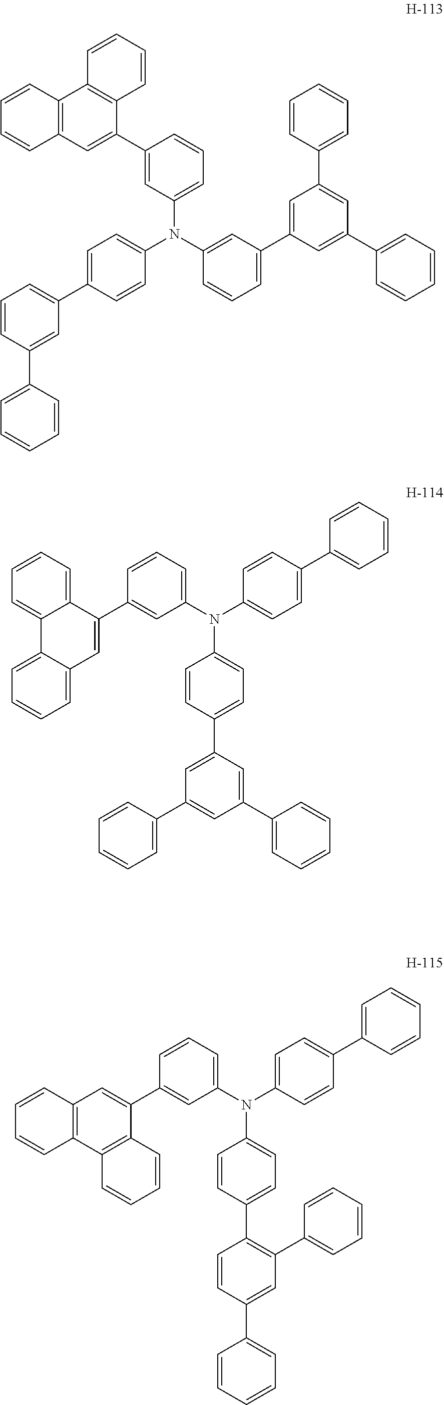 [SMILES: c1ccc(-c2cccc(-c3ccc(N(c4cccc(-c5cc(-c6ccccc6)cc(-c6ccccc6)c5)c4)c4cccc(-c5cc6ccccc6c6ccccc56)c4)cc3)c2)cc1 ; c1ccc(-c2ccc(N(c3ccc(-c4cc(-c5ccccc5)cc(-c5ccccc5)c4)cc3)c3cccc(-c4cc5ccccc5c5ccccc45)c3)cc2)cc1 ; c1ccc(-c2ccc(N(c3ccc(-c4ccc(-c5ccccc5)cc4-c4ccccc4)cc3)c3cccc(-c4cc5ccccc5c5ccccc45)c3)cc2)cc1]

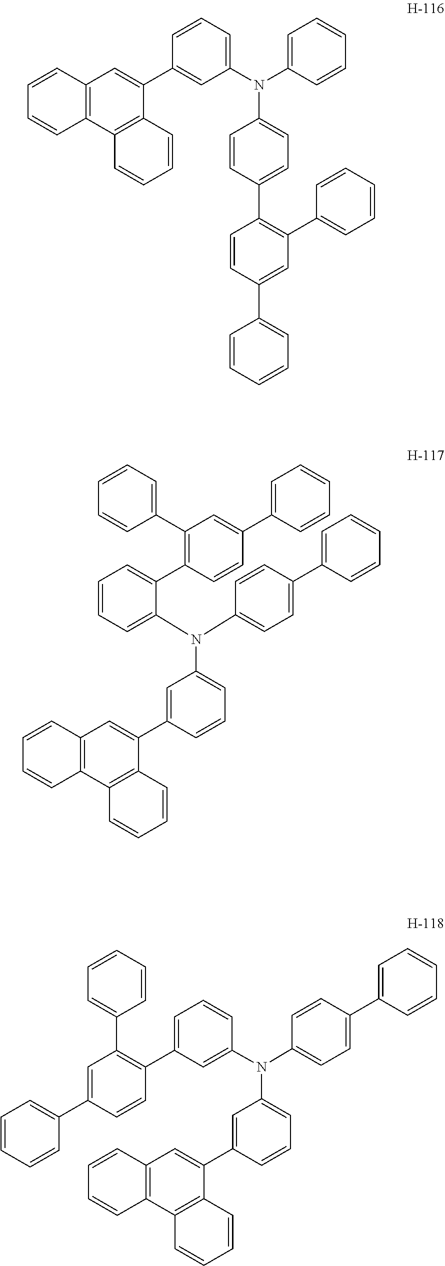 [SMILES: c1ccc(-c2ccc(-c3ccc(N(c4ccccc4)c4cccc(-c5cc6ccccc6c6ccccc56)c4)cc3)c(-c3ccccc3)c2)cc1 ; c1ccc(-c2ccc(N(c3cccc(-c4cc5ccccc5c5ccccc45)c3)c3ccccc3-c3ccc(-c4ccccc4)cc3-c3ccccc3)cc2)cc1 ; c1ccc(-c2ccc(N(c3cccc(-c4ccc(-c5ccccc5)cc4-c4ccccc4)c3)c3cccc(-c4cc5ccccc5c5ccccc45)c3)cc2)cc1]

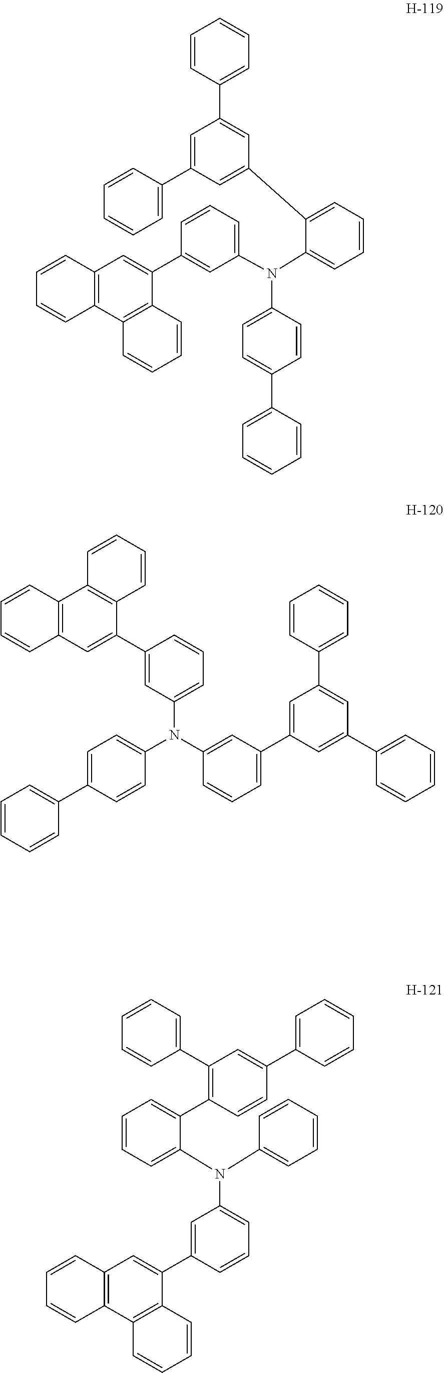 [SMILES: c1ccc(-c2ccc(N(c3cccc(-c4cc5ccccc5c5ccccc45)c3)c3ccccc3-c3cc(-c4ccccc4)cc(-c4ccccc4)c3)cc2)cc1 ; c1ccc(-c2ccc(N(c3cccc(-c4cc(-c5ccccc5)cc(-c5ccccc5)c4)c3)c3cccc(-c4cc5ccccc5c5ccccc45)c3)cc2)cc1 ; c1ccc(-c2ccc(-c3ccccc3N(c3ccccc3)c3cccc(-c4cc5ccccc5c5ccccc45)c3)c(-c3ccccc3)c2)cc1]

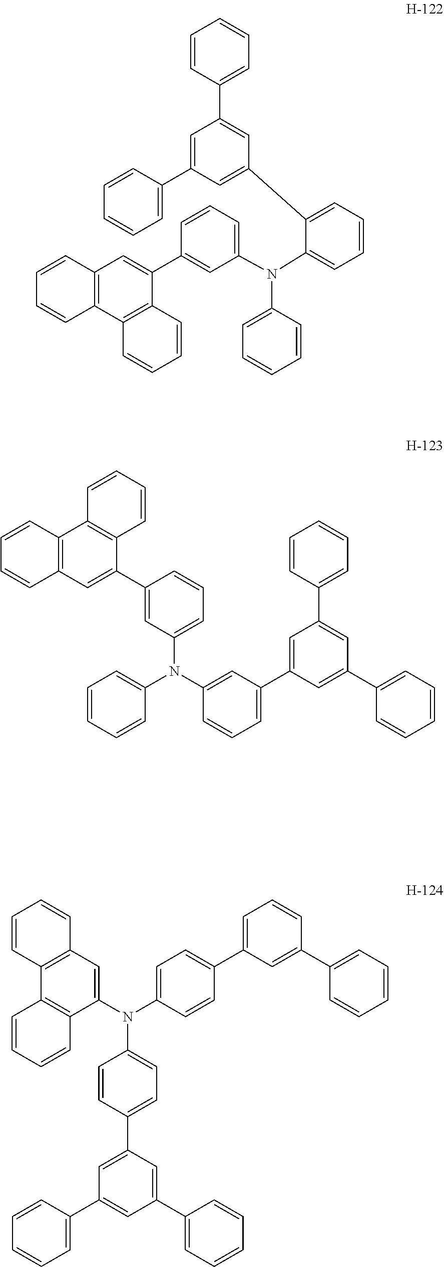 [SMILES: c1ccc(-c2cc(-c3ccccc3)cc(-c3ccccc3N(c3ccccc3)c3cccc(-c4cc5ccccc5c5ccccc45)c3)c2)cc1 ; c1ccc(-c2cc(-c3ccccc3)cc(-c3cccc(N(c4ccccc4)c4cccc(-c5cc6ccccc6c6ccccc56)c4)c3)c2)cc1 ; c1ccc(-c2cccc(-c3ccc(N(c4ccc(-c5cc(-c6ccccc6)cc(-c6ccccc6)c5)cc4)c4cc5ccccc5c5ccccc45)cc3)c2)cc1]

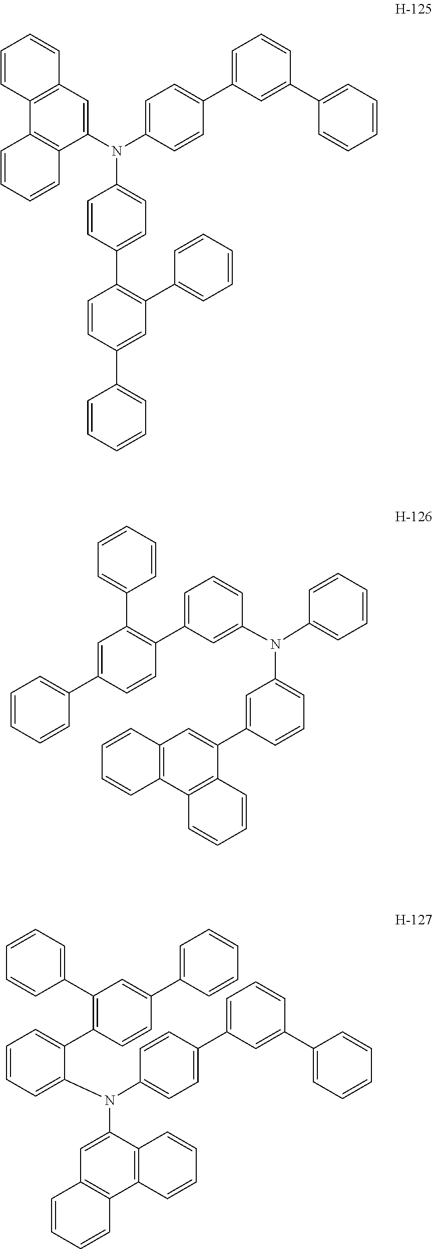 [SMILES: c1ccc(-c2cccc(-c3ccc(N(c4ccc(-c5ccc(-c6ccccc6)cc5-c5ccccc5)cc4)c4cc5ccccc5c5ccccc45)cc3)c2)cc1 ; c1ccc(-c2ccc(-c3cccc(N(c4ccccc4)c4cccc(-c5cc6ccccc6c6ccccc56)c4)c3)c(-c3ccccc3)c2)cc1 ; c1ccc(-c2cccc(-c3ccc(N(c4ccccc4-c4ccc(-c5ccccc5)cc4-c4ccccc4)c4cc5ccccc5c5ccccc45)cc3)c2)cc1]

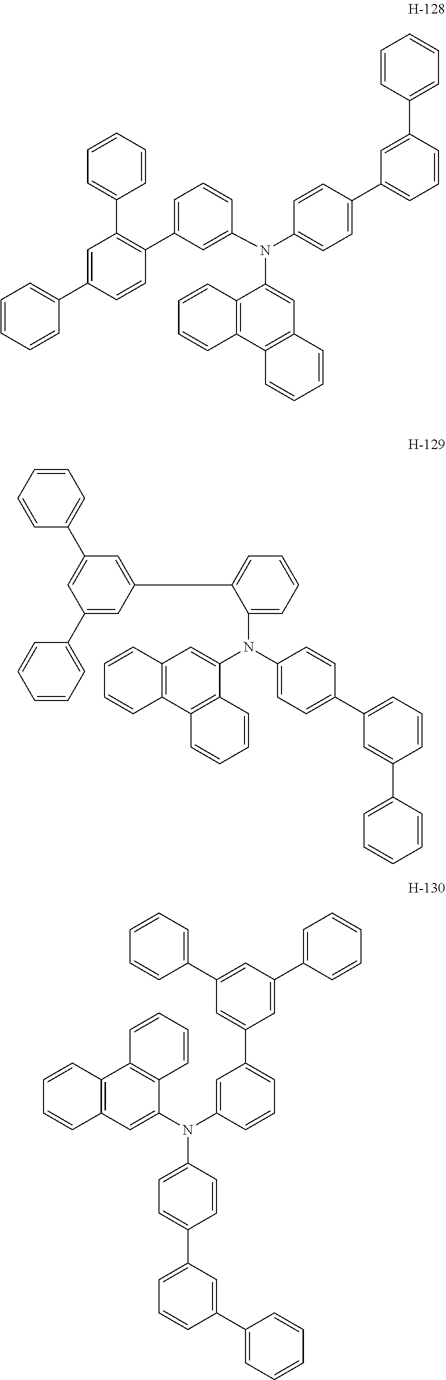 [SMILES: c1ccc(-c2cccc(-c3ccc(N(c4cccc(-c5ccc(-c6ccccc6)cc5-c5ccccc5)c4)c4cc5ccccc5c5ccccc45)cc3)c2)cc1 ; c1ccc(-c2cccc(-c3ccc(N(c4ccccc4-c4cc(-c5ccccc5)cc(-c5ccccc5)c4)c4cc5ccccc5c5ccccc45)cc3)c2)cc1 ; c1ccc(-c2cccc(-c3ccc(N(c4cccc(-c5cc(-c6ccccc6)cc(-c6ccccc6)c5)c4)c4cc5ccccc5c5ccccc45)cc3)c2)cc1]

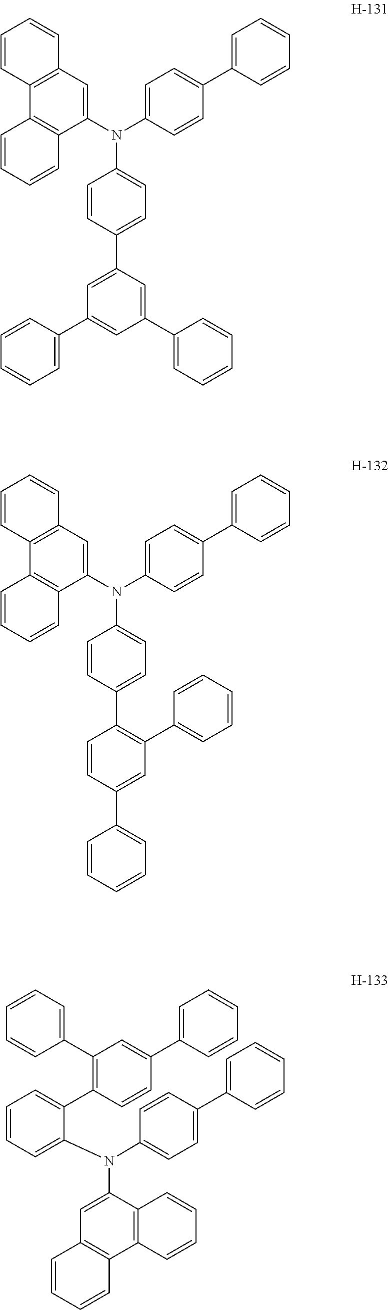 [SMILES: c1ccc(-c2ccc(N(c3ccc(-c4cc(-c5ccccc5)cc(-c5ccccc5)c4)cc3)c3cc4ccccc4c4ccccc34)cc2)cc1 ; c1ccc(-c2ccc(N(c3ccc(-c4ccc(-c5ccccc5)cc4-c4ccccc4)cc3)c3cc4ccccc4c4ccccc34)cc2)cc1 ; c1ccc(-c2ccc(N(c3ccccc3-c3ccc(-c4ccccc4)cc3-c3ccccc3)c3cc4ccccc4c4ccccc34)cc2)cc1]

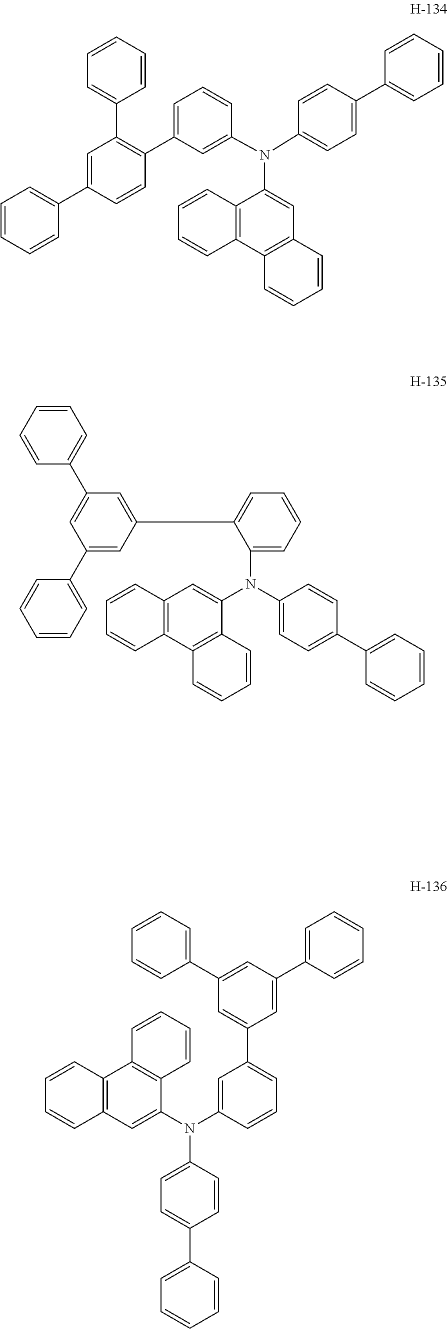 [SMILES: c1ccc(-c2ccc(N(c3cccc(-c4ccc(-c5ccccc5)cc4-c4ccccc4)c3)c3cc4ccccc4c4ccccc34)cc2)cc1 ; c1ccc(-c2ccc(N(c3ccccc3-c3cc(-c4ccccc4)cc(-c4ccccc4)c3)c3cc4ccccc4c4ccccc34)cc2)cc1 ; c1ccc(-c2ccc(N(c3cccc(-c4cc(-c5ccccc5)cc(-c5ccccc5)c4)c3)c3cc4ccccc4c4ccccc34)cc2)cc1]

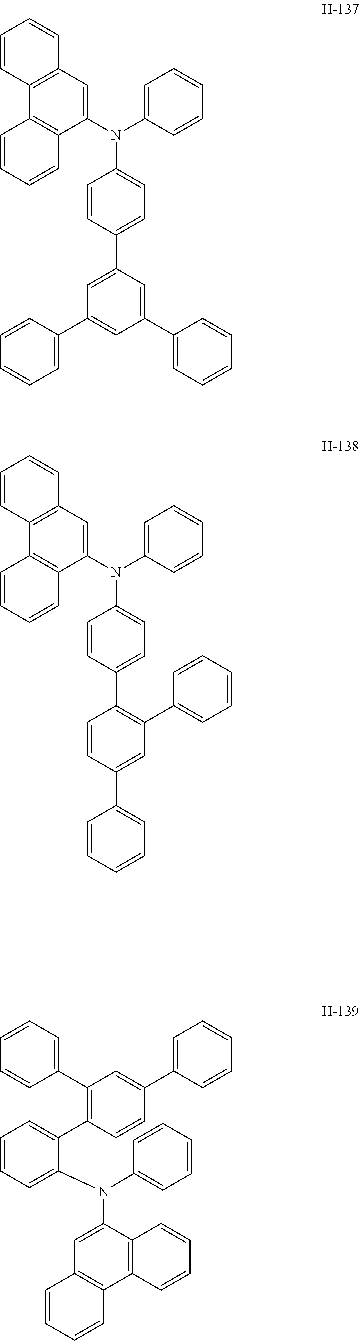 [SMILES: c1ccc(-c2cc(-c3ccccc3)cc(-c3ccc(N(c4ccccc4)c4cc5ccccc5c5ccccc45)cc3)c2)cc1 ; c1ccc(-c2ccc(-c3ccc(N(c4ccccc4)c4cc5ccccc5c5ccccc45)cc3)c(-c3ccccc3)c2)cc1 ; c1ccc(-c2ccc(-c3ccccc3N(c3ccccc3)c3cc4ccccc4c4ccccc34)c(-c3ccccc3)c2)cc1]

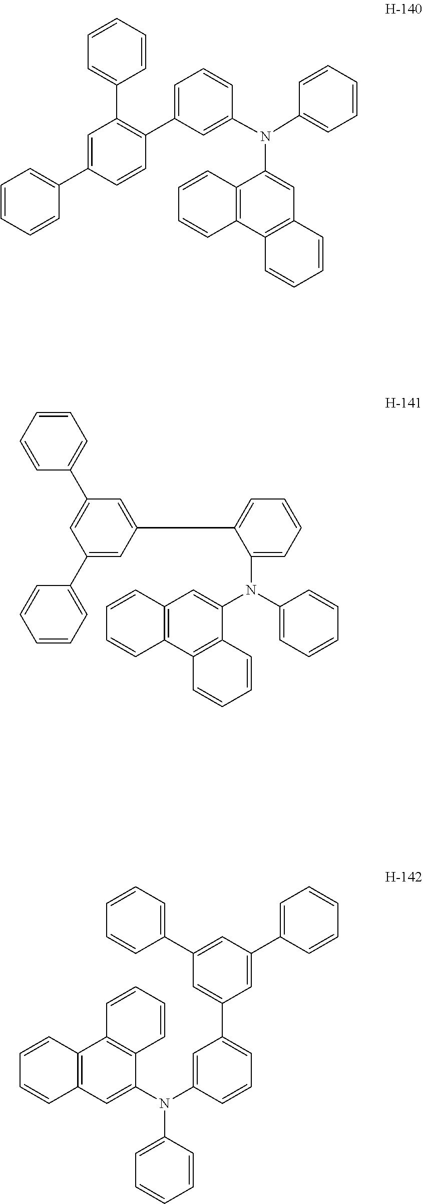 [SMILES: c1ccc(-c2ccc(-c3cccc(N(c4ccccc4)c4cc5ccccc5c5ccccc45)c3)c(-c3ccccc3)c2)cc1 ; c1ccc(-c2cc(-c3ccccc3)cc(-c3ccccc3N(c3ccccc3)c3cc4ccccc4c4ccccc34)c2)cc1 ; c1ccc(-c2cc(-c3ccccc3)cc(-c3cccc(N(c4ccccc4)c4cc5ccccc5c5ccccc45)c3)c2)cc1]

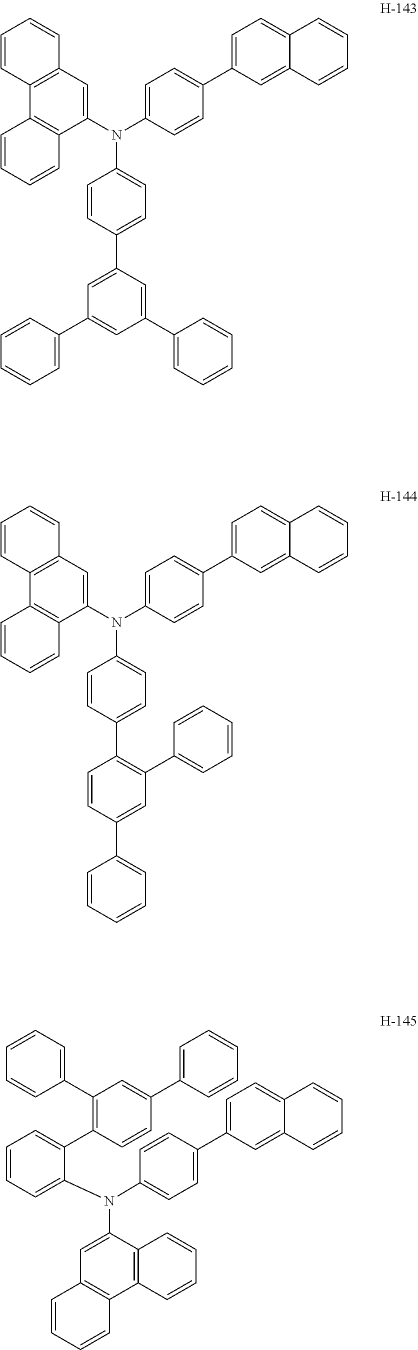 [SMILES: c1ccc(-c2cc(-c3ccccc3)cc(-c3ccc(N(c4ccc(-c5ccc6ccccc6c5)cc4)c4cc5ccccc5c5ccccc45)cc3)c2)cc1 ; c1ccc(-c2ccc(-c3ccc(N(c4ccc(-c5ccc6ccccc6c5)cc4)c4cc5ccccc5c5ccccc45)cc3)c(-c3ccccc3)c2)cc1 ; c1ccc(-c2ccc(-c3ccccc3N(c3ccc(-c4ccc5ccccc5c4)cc3)c3cc4ccccc4c4ccccc34)c(-c3ccccc3)c2)cc1]

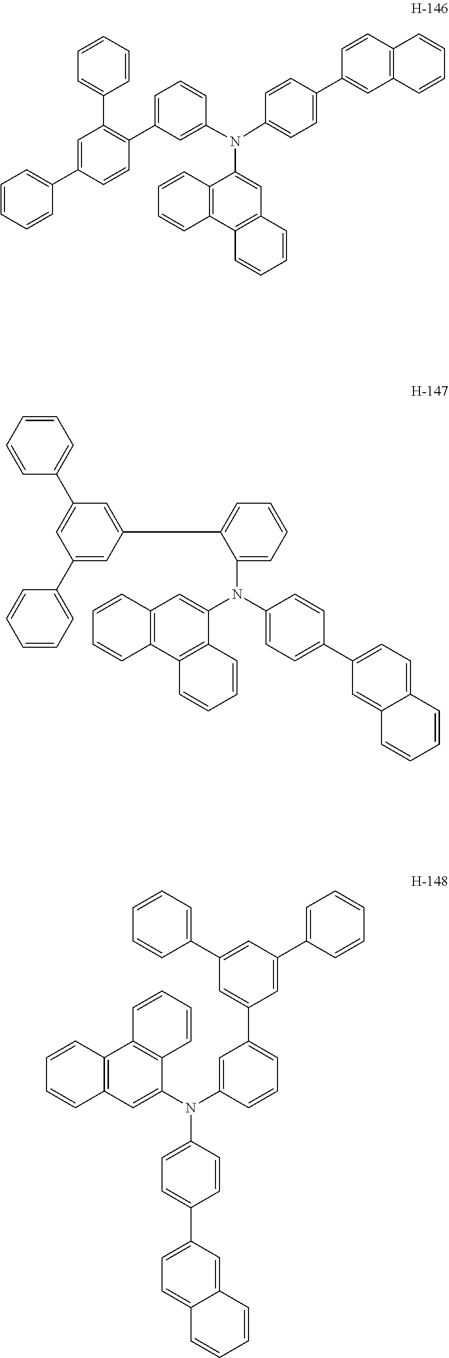 [SMILES: c1ccc(-c2ccc(-c3cccc(N(c4ccc(-c5ccc6ccccc6c5)cc4)c4cc5ccccc5c5ccccc45)c3)c(-c3ccccc3)c2)cc1 ; c1ccc(-c2cc(-c3ccccc3)cc(-c3ccccc3N(c3ccc(-c4ccc5ccccc5c4)cc3)c3cc4ccccc4c4ccccc34)c2)cc1 ; c1ccc(-c2cc(-c3ccccc3)cc(-c3cccc(N(c4ccc(-c5ccc6ccccc6c5)cc4)c4cc5ccccc5c5ccccc45)c3)c2)cc1]

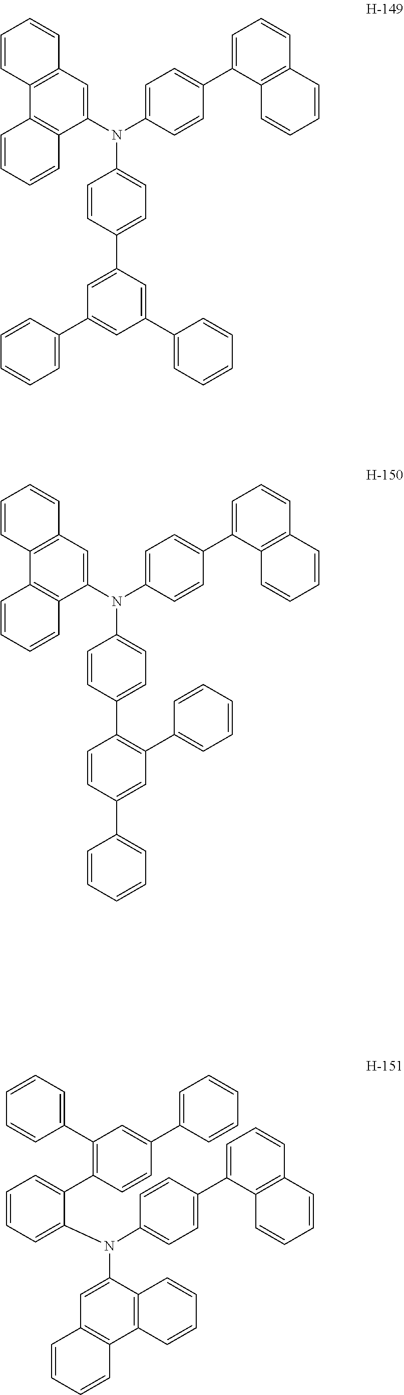 [SMILES: c1ccc(-c2cc(-c3ccccc3)cc(-c3ccc(N(c4ccc(-c5cccc6ccccc56)cc4)c4cc5ccccc5c5ccccc45)cc3)c2)cc1 ; c1ccc(-c2ccc(-c3ccc(N(c4ccc(-c5cccc6ccccc56)cc4)c4cc5ccccc5c5ccccc45)cc3)c(-c3ccccc3)c2)cc1 ; c1ccc(-c2ccc(-c3ccccc3N(c3ccc(-c4cccc5ccccc45)cc3)c3cc4ccccc4c4ccccc34)c(-c3ccccc3)c2)cc1]

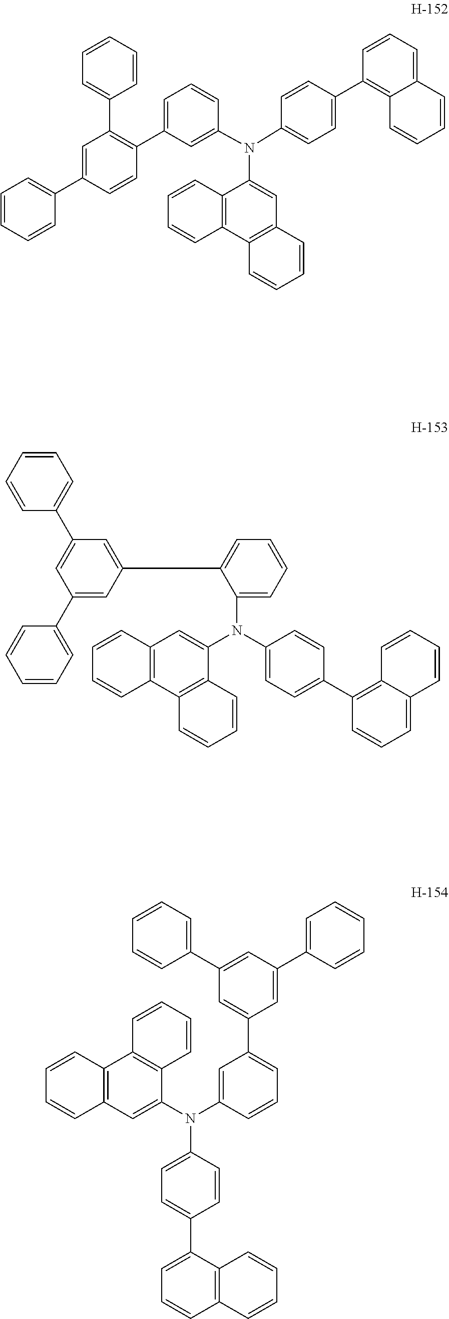 [SMILES: c1ccc(-c2ccc(-c3cccc(N(c4ccc(-c5cccc6ccccc56)cc4)c4cc5ccccc5c5ccccc45)c3)c(-c3ccccc3)c2)cc1 ; c1ccc(-c2cc(-c3ccccc3)cc(-c3ccccc3N(c3ccc(-c4cccc5ccccc45)cc3)c3cc4ccccc4c4ccccc34)c2)cc1 ; c1ccc(-c2cc(-c3ccccc3)cc(-c3cccc(N(c4ccc(-c5cccc6ccccc56)cc4)c4cc5ccccc5c5ccccc45)c3)c2)cc1]

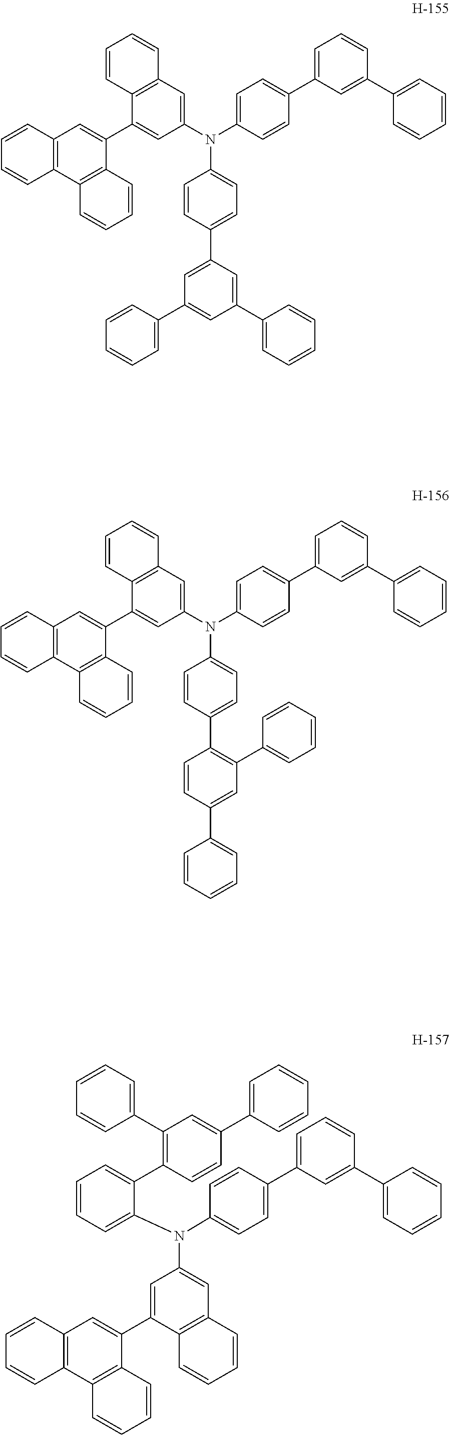 [SMILES: c1ccc(-c2cccc(-c3ccc(N(c4ccc(-c5cc(-c6ccccc6)cc(-c6ccccc6)c5)cc4)c4cc(-c5cc6ccccc6c6ccccc56)c5ccccc5c4)cc3)c2)cc1 ; c1ccc(-c2cccc(-c3ccc(N(c4ccc(-c5ccc(-c6ccccc6)cc5-c5ccccc5)cc4)c4cc(-c5cc6ccccc6c6ccccc56)c5ccccc5c4)cc3)c2)cc1 ; c1ccc(-c2cccc(-c3ccc(N(c4cc(-c5cc6ccccc6c6ccccc56)c5ccccc5c4)c4ccccc4-c4ccc(-c5ccccc5)cc4-c4ccccc4)cc3)c2)cc1]

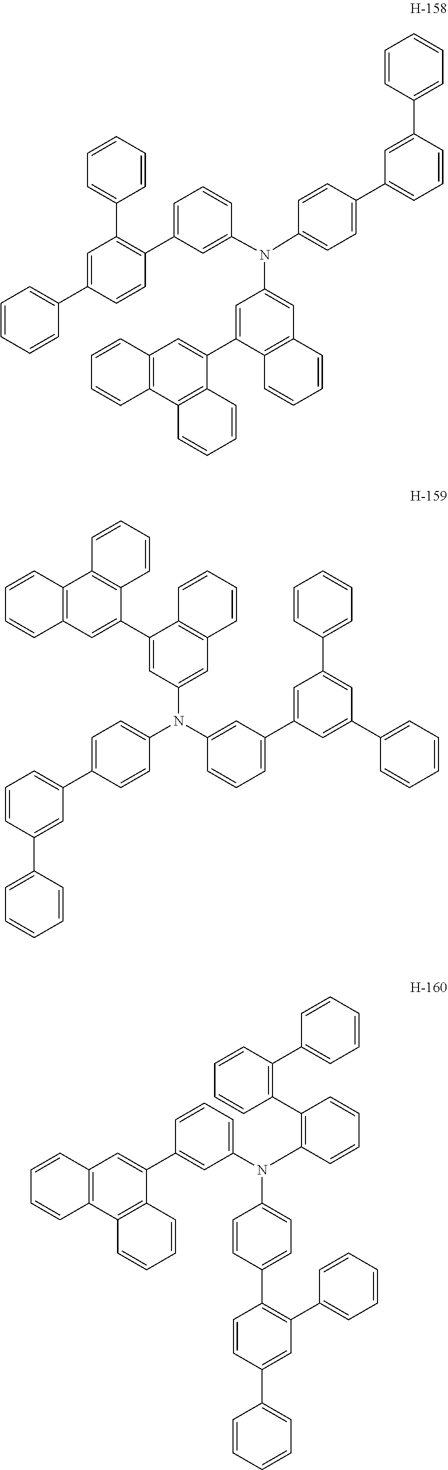 [SMILES: c1ccc(-c2cccc(-c3ccc(N(c4cccc(-c5ccc(-c6ccccc6)cc5-c5ccccc5)c4)c4cc(-c5cc6ccccc6c6ccccc56)c5ccccc5c4)cc3)c2)cc1 ; c1ccc(-c2cccc(-c3ccc(N(c4cccc(-c5cc(-c6ccccc6)cc(-c6ccccc6)c5)c4)c4cc(-c5cc6ccccc6c6ccccc56)c5ccccc5c4)cc3)c2)cc1 ; c1ccc(-c2ccc(-c3ccc(N(c4cccc(-c5cc6ccccc6c6ccccc56)c4)c4ccccc4-c4ccccc4-c4ccccc4)cc3)c(-c3ccccc3)c2)cc1]

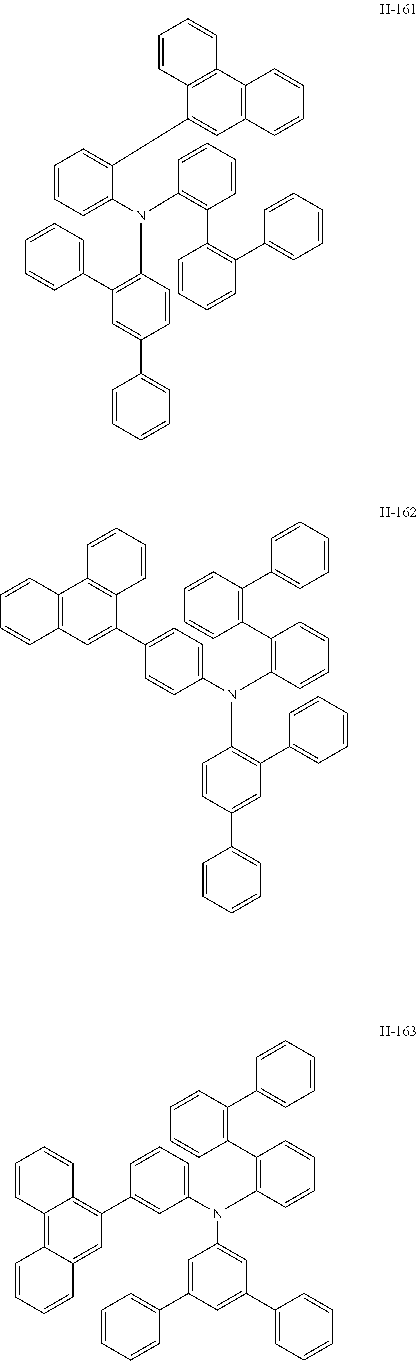 [SMILES: c1ccc(-c2ccc(N(c3ccccc3-c3ccccc3-c3ccccc3)c3ccccc3-c3cc4ccccc4c4ccccc34)c(-c3ccccc3)c2)cc1 ; c1ccc(-c2ccc(N(c3ccc(-c4cc5ccccc5c5ccccc45)cc3)c3ccccc3-c3ccccc3-c3ccccc3)c(-c3ccccc3)c2)cc1 ; c1ccc(-c2cc(-c3ccccc3)cc(N(c3cccc(-c4cc5ccccc5c5ccccc45)c3)c3ccccc3-c3ccccc3-c3ccccc3)c2)cc1]

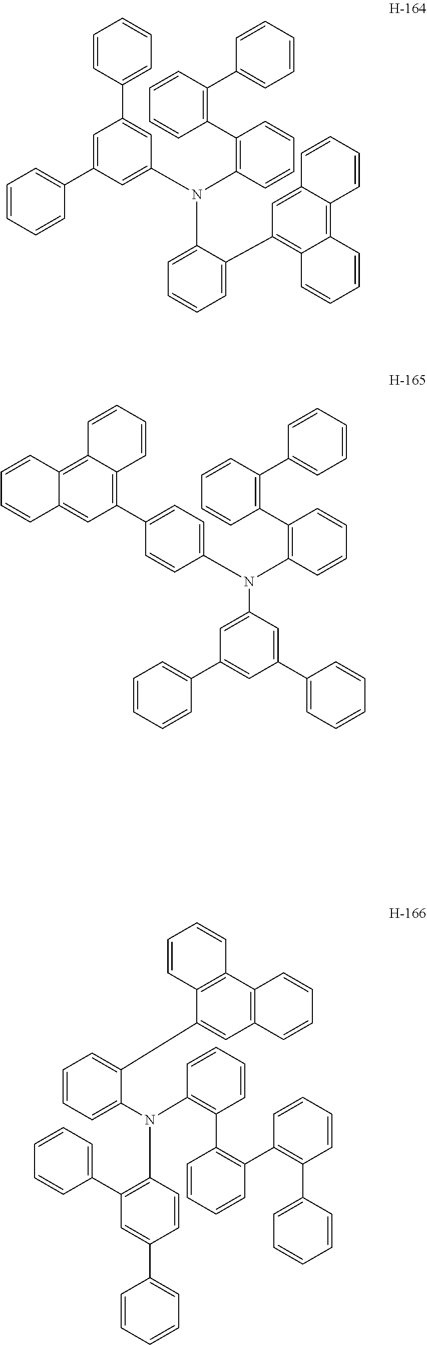 [SMILES: c1ccc(-c2cc(-c3ccccc3)cc(N(c3ccccc3-c3ccccc3-c3ccccc3)c3ccccc3-c3cc4ccccc4c4ccccc34)c2)cc1 ; c1ccc(-c2cc(-c3ccccc3)cc(N(c3ccc(-c4cc5ccccc5c5ccccc45)cc3)c3ccccc3-c3ccccc3-c3ccccc3)c2)cc1 ; c1ccc(-c2ccc(N(c3ccccc3-c3ccccc3-c3ccccc3-c3ccccc3)c3ccccc3-c3cc4ccccc4c4ccccc34)c(-c3ccccc3)c2)cc1]

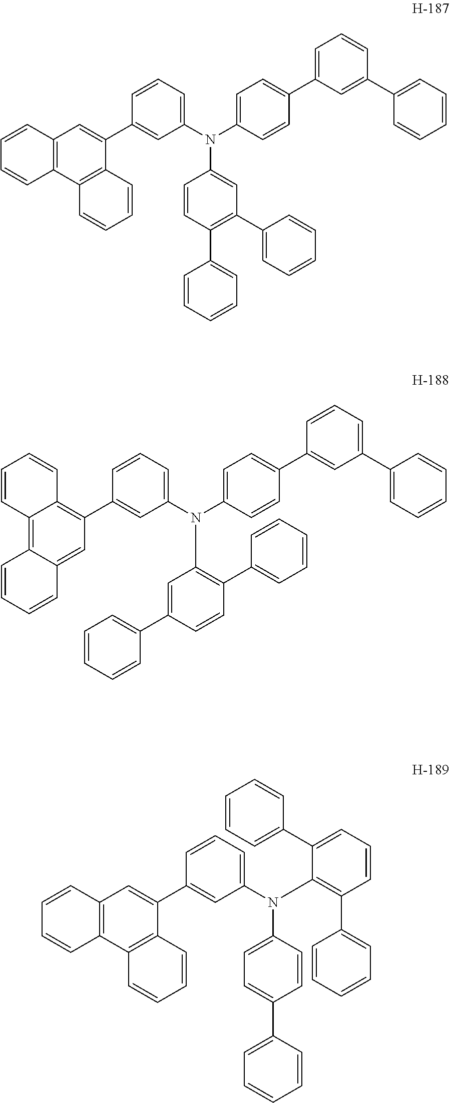 [SMILES: c1ccc(-c2cccc(-c3ccc(N(c4cccc(-c5cc6ccccc6c6ccccc56)c4)c4ccc(-c5ccccc5)c(-c5ccccc5)c4)cc3)c2)cc1 ; c1ccc(-c2cccc(-c3ccc(N(c4cccc(-c5cc6ccccc6c6ccccc56)c4)c4cc(-c5ccccc5)ccc4-c4ccccc4)cc3)c2)cc1 ; c1ccc(-c2ccc(N(c3cccc(-c4cc5ccccc5c5ccccc45)c3)c3c(-c4ccccc4)cccc3-c3ccccc3)cc2)cc1]

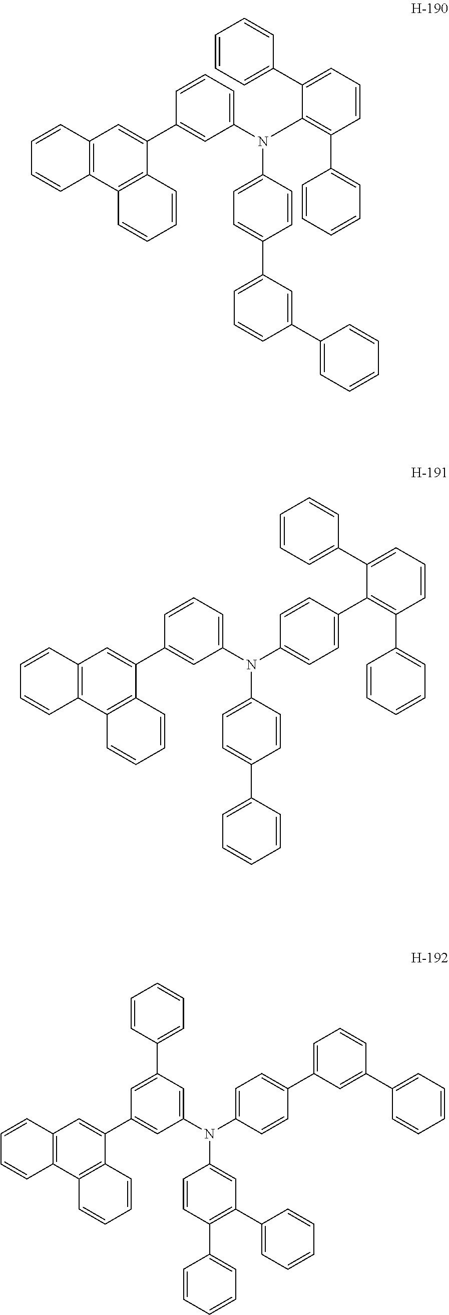 [SMILES: c1ccc(-c2cccc(-c3ccc(N(c4cccc(-c5cc6ccccc6c6ccccc56)c4)c4c(-c5ccccc5)cccc4-c4ccccc4)cc3)c2)cc1 ; c1ccc(-c2ccc(N(c3ccc(-c4c(-c5ccccc5)cccc4-c4ccccc4)cc3)c3cccc(-c4cc5ccccc5c5ccccc45)c3)cc2)cc1 ; c1ccc(-c2cccc(-c3ccc(N(c4cc(-c5ccccc5)cc(-c5cc6ccccc6c6ccccc56)c4)c4ccc(-c5ccccc5)c(-c5ccccc5)c4)cc3)c2)cc1]

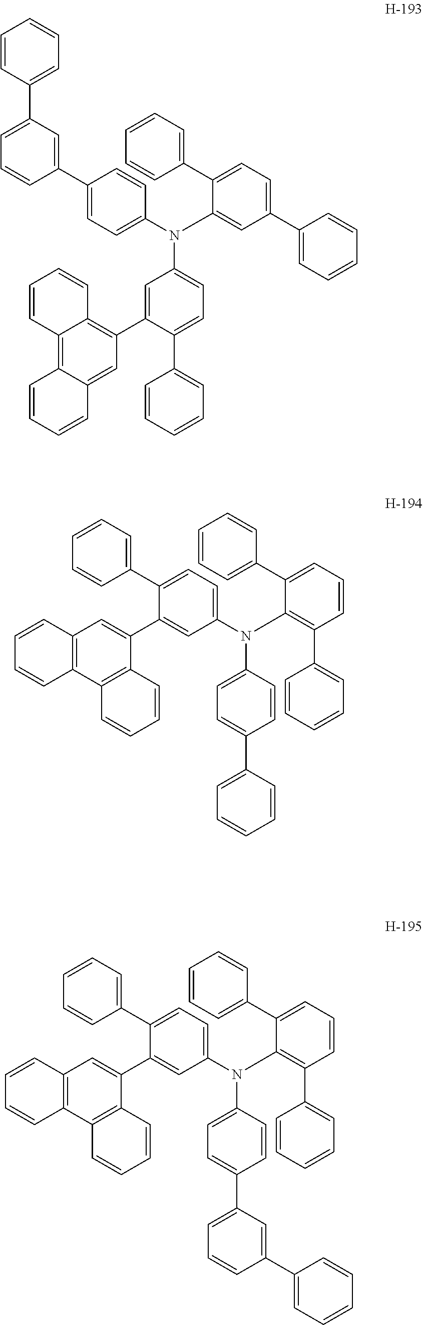 [SMILES: c1ccc(-c2cccc(-c3ccc(N(c4ccc(-c5ccccc5)c(-c5cc6ccccc6c6ccccc56)c4)c4cc(-c5ccccc5)ccc4-c4ccccc4)cc3)c2)cc1 ; c1ccc(-c2ccc(N(c3ccc(-c4ccccc4)c(-c4cc5ccccc5c5ccccc45)c3)c3c(-c4ccccc4)cccc3-c3ccccc3)cc2)cc1 ; c1ccc(-c2cccc(-c3ccc(N(c4ccc(-c5ccccc5)c(-c5cc6ccccc6c6ccccc56)c4)c4c(-c5ccccc5)cccc4-c4ccccc4)cc3)c2)cc1]

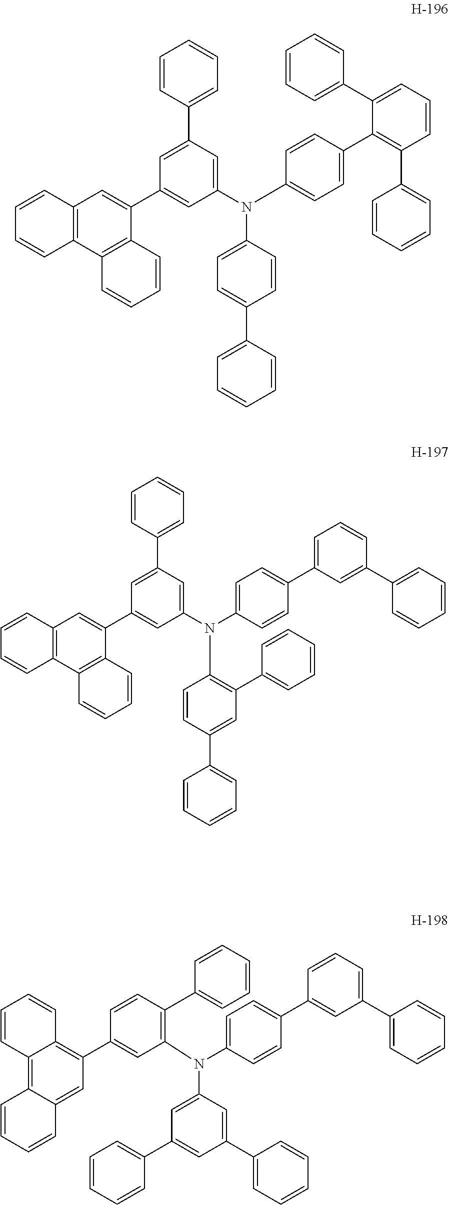 [SMILES: c1ccc(-c2ccc(N(c3ccc(-c4c(-c5ccccc5)cccc4-c4ccccc4)cc3)c3cc(-c4ccccc4)cc(-c4cc5ccccc5c5ccccc45)c3)cc2)cc1 ; c1ccc(-c2cccc(-c3ccc(N(c4cc(-c5ccccc5)cc(-c5cc6ccccc6c6ccccc56)c4)c4ccc(-c5ccccc5)cc4-c4ccccc4)cc3)c2)cc1 ; c1ccc(-c2cccc(-c3ccc(N(c4cc(-c5ccccc5)cc(-c5ccccc5)c4)c4cc(-c5cc6ccccc6c6ccccc56)ccc4-c4ccccc4)cc3)c2)cc1]

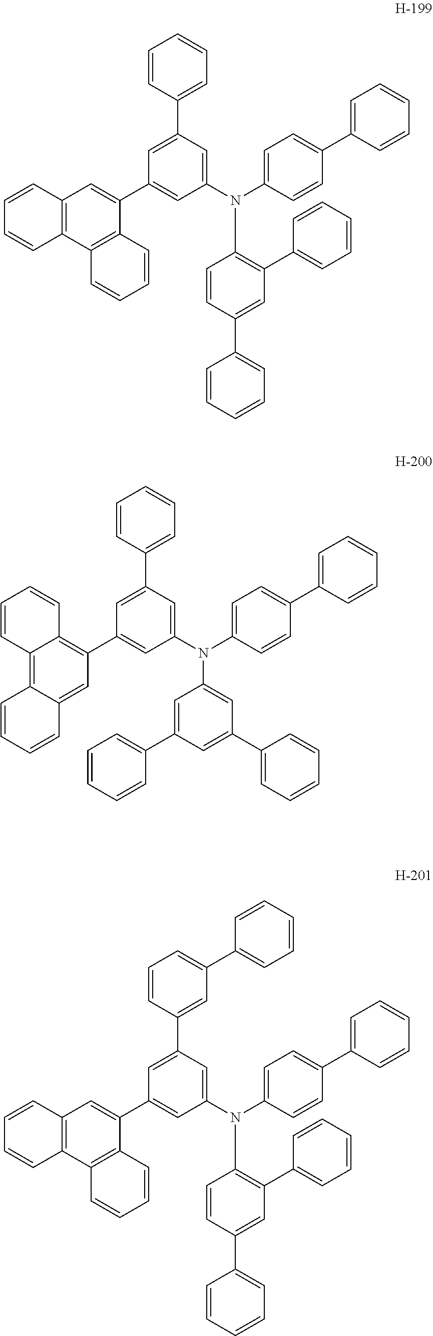 [SMILES: c1ccc(-c2ccc(N(c3cc(-c4ccccc4)cc(-c4cc5ccccc5c5ccccc45)c3)c3ccc(-c4ccccc4)cc3-c3ccccc3)cc2)cc1 ; c1ccc(-c2ccc(N(c3cc(-c4ccccc4)cc(-c4ccccc4)c3)c3cc(-c4ccccc4)cc(-c4cc5ccccc5c5ccccc45)c3)cc2)cc1 ; c1ccc(-c2ccc(N(c3cc(-c4cccc(-c5ccccc5)c4)cc(-c4cc5ccccc5c5ccccc45)c3)c3ccc(-c4ccccc4)cc3-c3ccccc3)cc2)cc1]

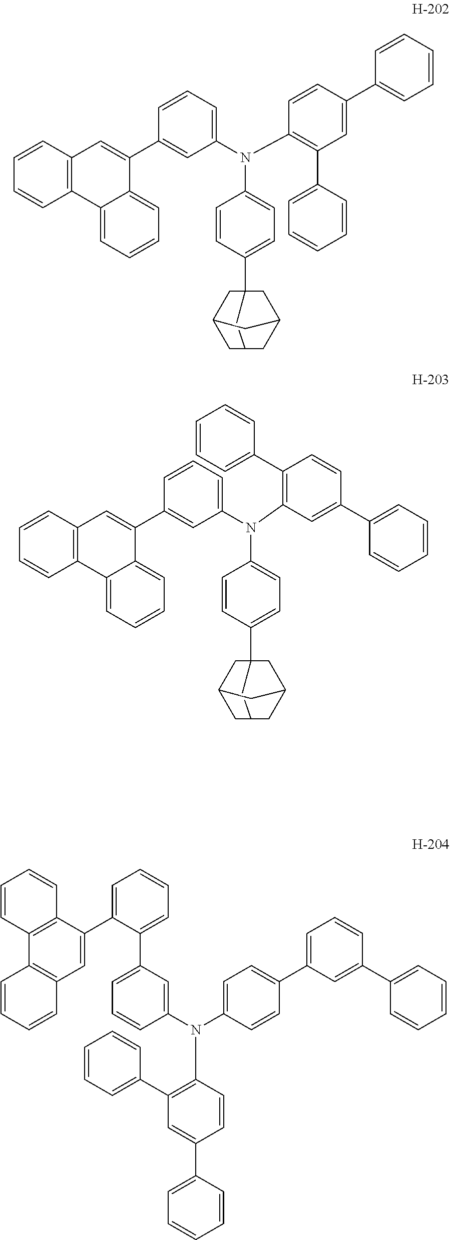 [SMILES: c1ccc(-c2ccc(N(c3ccc(C45CC6CC(CC(C6)C4)C5)cc3)c3cccc(-c4cc5ccccc5c5ccccc45)c3)c(-c3ccccc3)c2)cc1 ; c1ccc(-c2ccc(-c3ccccc3)c(N(c3ccc(C45CC6CC(CC(C6)C4)C5)cc3)c3cccc(-c4cc5ccccc5c5ccccc45)c3)c2)cc1 ; c1ccc(-c2cccc(-c3ccc(N(c4cccc(-c5ccccc5-c5cc6ccccc6c6ccccc56)c4)c4ccc(-c5ccccc5)cc4-c4ccccc4)cc3)c2)cc1]

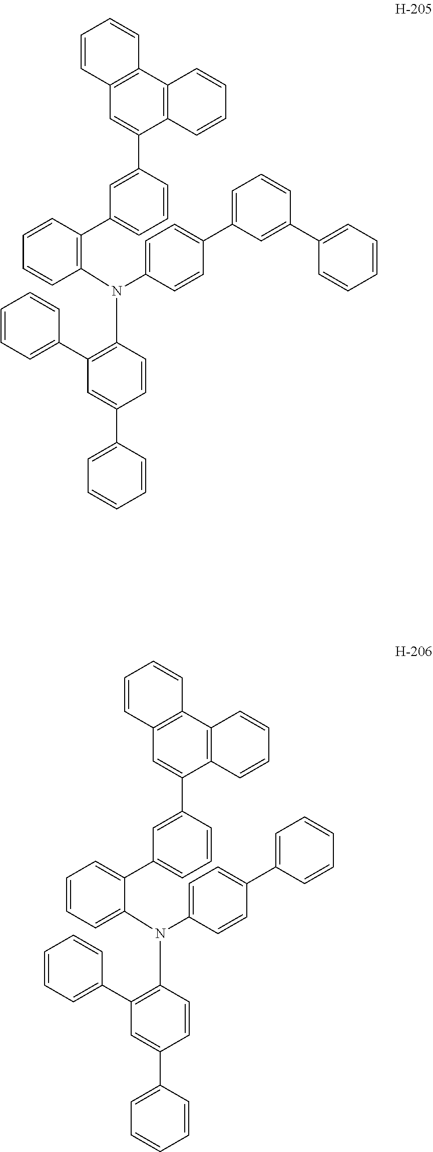 [SMILES: c1ccc(-c2cccc(-c3ccc(N(c4ccccc4-c4cccc(-c5cc6ccccc6c6ccccc56)c4)c4ccc(-c5ccccc5)cc4-c4ccccc4)cc3)c2)cc1 ; c1ccc(-c2ccc(N(c3ccccc3-c3cccc(-c4cc5ccccc5c5ccccc45)c3)c3ccc(-c4ccccc4)cc3-c3ccccc3)cc2)cc1]

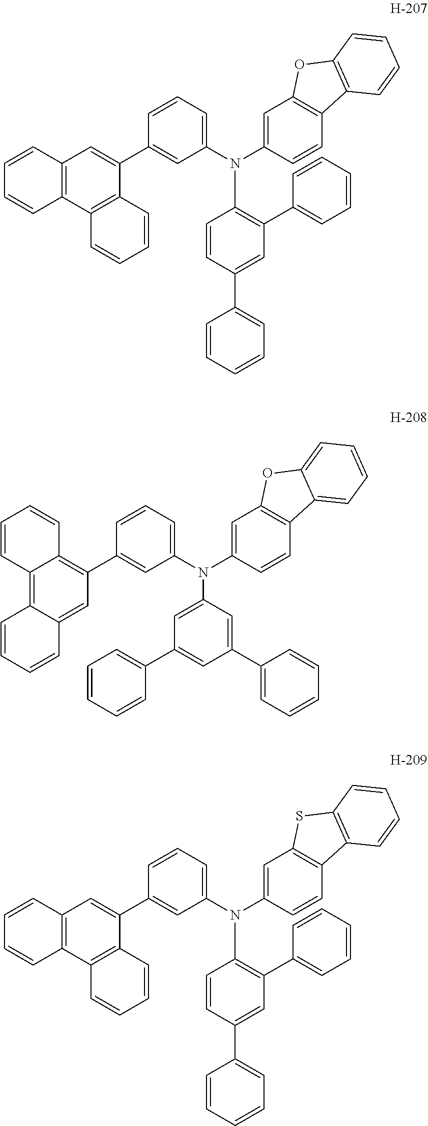 [SMILES: c1ccc(-c2ccc(N(c3cccc(-c4cc5ccccc5c5ccccc45)c3)c3ccc4c(c3)oc3ccccc34)c(-c3ccccc3)c2)cc1 ; c1ccc(-c2cc(-c3ccccc3)cc(N(c3cccc(-c4cc5ccccc5c5ccccc45)c3)c3ccc4c(c3)oc3ccccc34)c2)cc1 ; c1ccc(-c2ccc(N(c3cccc(-c4cc5ccccc5c5ccccc45)c3)c3ccc4c(c3)sc3ccccc34)c(-c3ccccc3)c2)cc1]

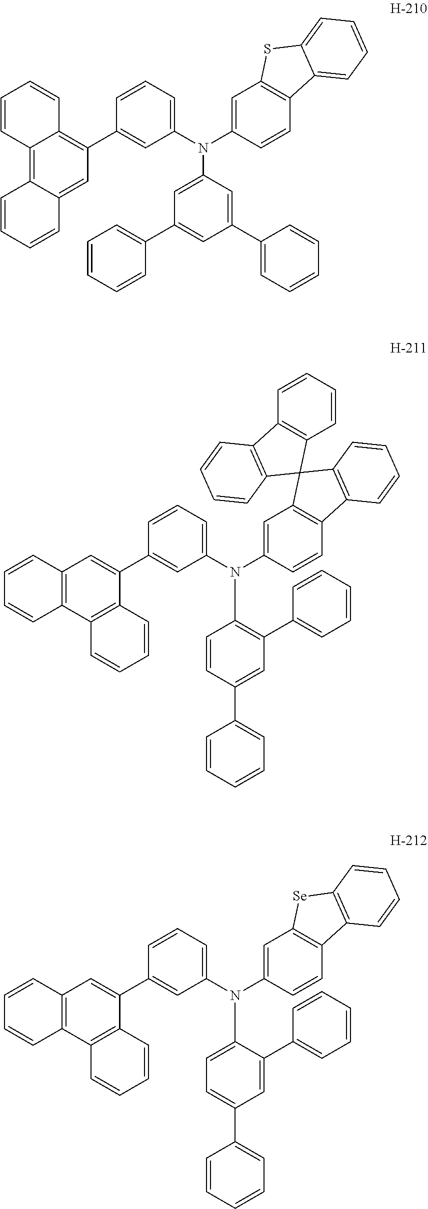 [SMILES: c1ccc(-c2cc(-c3ccccc3)cc(N(c3cccc(-c4cc5ccccc5c5ccccc45)c3)c3ccc4c(c3)sc3ccccc34)c2)cc1 ; c1ccc(-c2ccc(N(c3cccc(-c4cc5ccccc5c5ccccc45)c3)c3ccc4c(c3)C3(c5ccccc5-c5ccccc53)c3ccccc3-4)c(-c3ccccc3)c2)cc1 ; c1ccc(-c2ccc(N(c3cccc(-c4cc5ccccc5c5ccccc45)c3)c3ccc4c(c3)[se]c3ccccc34)c(-c3ccccc3)c2)cc1]

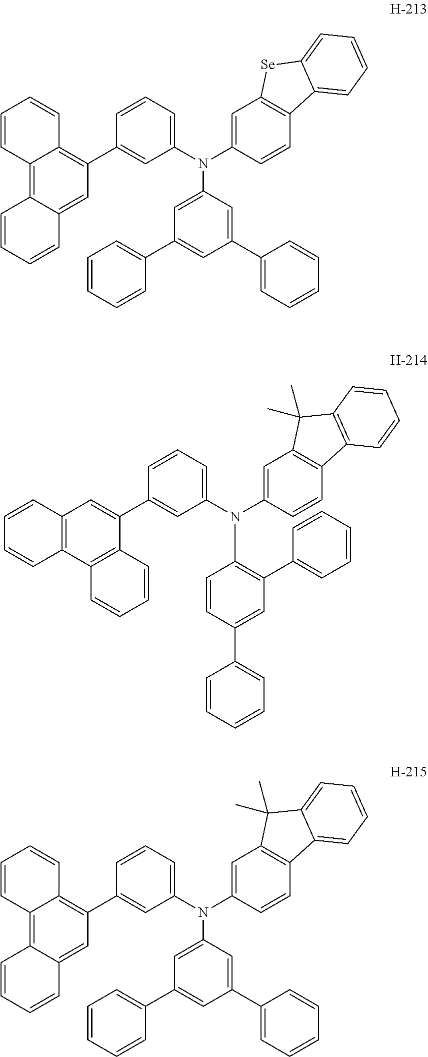 [SMILES: c1ccc(-c2cc(-c3ccccc3)cc(N(c3cccc(-c4cc5ccccc5c5ccccc45)c3)c3ccc4c(c3)[se]c3ccccc34)c2)cc1 ; CC1(C)c2ccccc2-c2ccc(N(c3cccc(-c4cc5ccccc5c5ccccc45)c3)c3ccc(-c4ccccc4)cc3-c3ccccc3)cc21 ; CC1(C)c2ccccc2-c2ccc(N(c3cc(-c4ccccc4)cc(-c4ccccc4)c3)c3cccc(-c4cc5ccccc5c5ccccc45)c3)cc21]

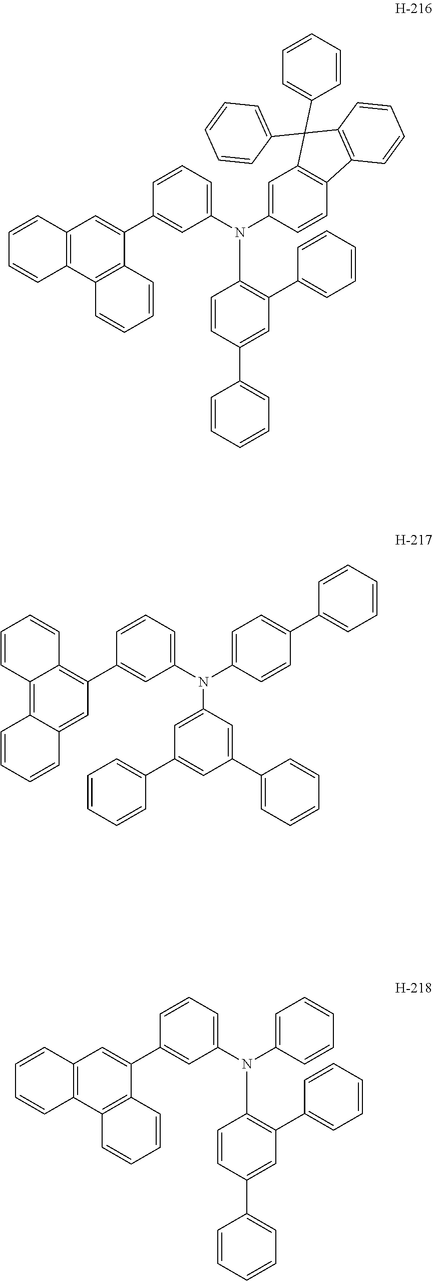 [SMILES: c1ccc(-c2ccc(N(c3cccc(-c4cc5ccccc5c5ccccc45)c3)c3ccc4c(c3)C(c3ccccc3)(c3ccccc3)c3ccccc3-4)c(-c3ccccc3)c2)cc1 ; c1ccc(-c2ccc(N(c3cc(-c4ccccc4)cc(-c4ccccc4)c3)c3cccc(-c4cc5ccccc5c5ccccc45)c3)cc2)cc1 ; c1ccc(-c2ccc(N(c3ccccc3)c3cccc(-c4cc5ccccc5c5ccccc45)c3)c(-c3ccccc3)c2)cc1]

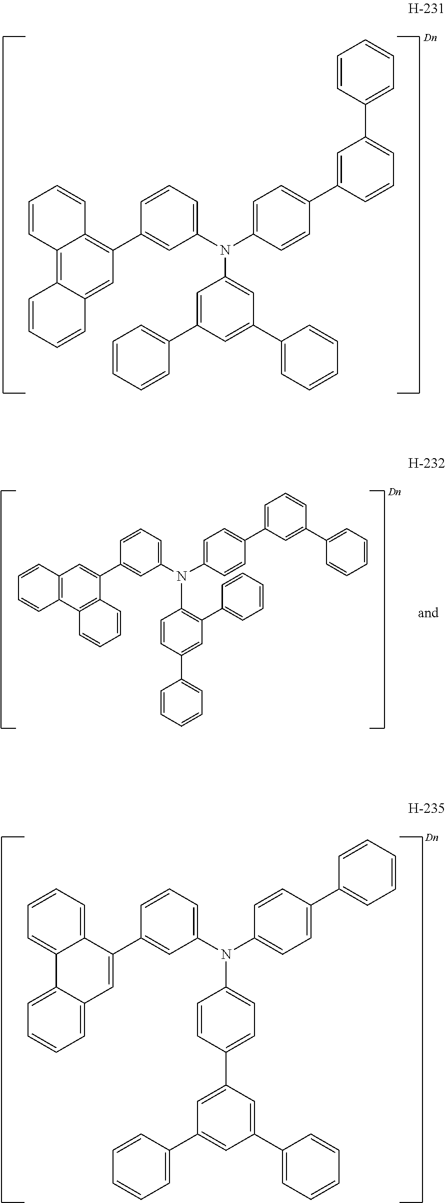

Wherein in the compounds above, Dn represents that n number of hydrogens are replaced with deuterium, and n represents an integer of 1 or more, which is an integer of 1 to the maximum number of hydrogens in the compound.

Examples of the production method for the compound represented by Formula 3 according to the present disclosure are shown in the following Reaction Scheme 2 and Reaction Scheme 3, but are not limited thereto. Such compound may be produced by way of synthetic methods known to one skilled in the art.

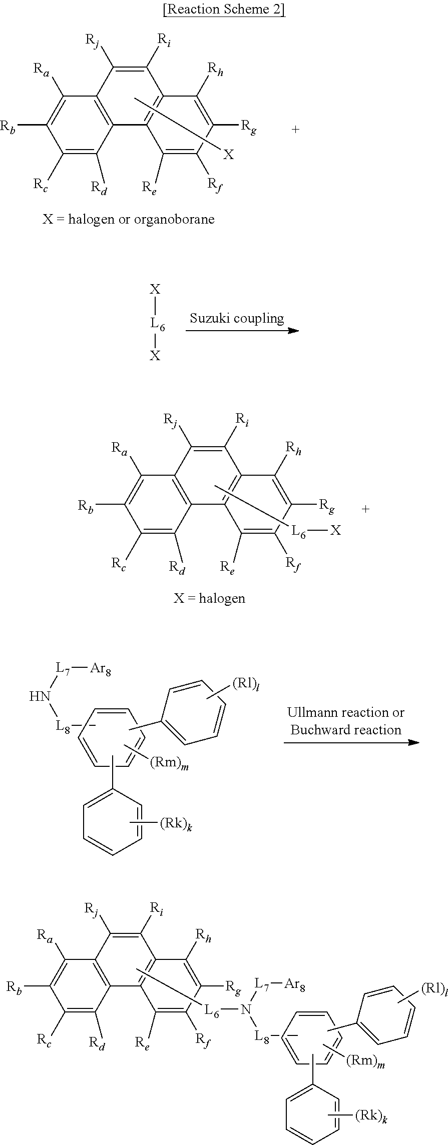

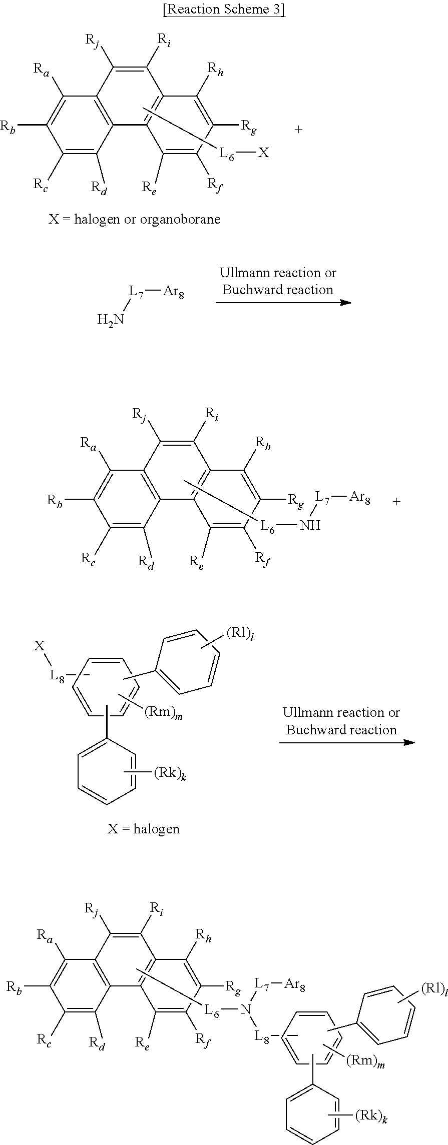

In the above Reaction Scheme 2 and Reaction Scheme 3, Rₐ to Rₘ, L₆ to L₈, Ar₈, and k to m are as defined in Formula 3 and Formula 3-1.

The organic electroluminescent device according to the present disclosure may comprise the organic electroluminescent compound represented by Formula 3.

The light-emitting layer may include one or more hosts and one or more dopants. If necessary, the light-emitting layer may include a co-host material, that is, a plurality of host materials. The host used in the present invention may be a phosphorescent host compound or a fluorescent host compound, and these host compounds are not particularly limited thereto.

According to one embodiment of the present disclosure, the doping concentration of the dopant compound with respect to the host compound of the light-emitting layer may be less than 20 wt %. The dopant comprised in the organic electroluminescent device of the present disclosure may be at least one phosphorescent or fluorescent dopant, and is preferably a phosphorescent dopant. The phosphorescent dopant materials applied to the organic electroluminescent device according to the present disclosure are not particularly limited, but may be a metallated complex compound of a metal atom selected from iridium (Ir), osmium (Os), copper (Cu), and platinum (Pt), preferably an ortho-metallated complex compound of a metal atom selected from iridium (Ir), osmium (Os), copper (Cu), and platinum (Pt), and more preferably an ortho-metallated iridium complex compound.

The dopant comprised in the organic electroluminescent device of the present disclosure may be a compound represented by the following Formula 101, but is not limited thereto.

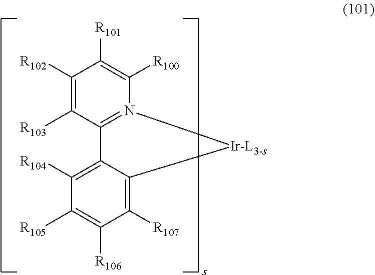

In Formula 101,
L is any one selected from the following Structures 1 to 3:

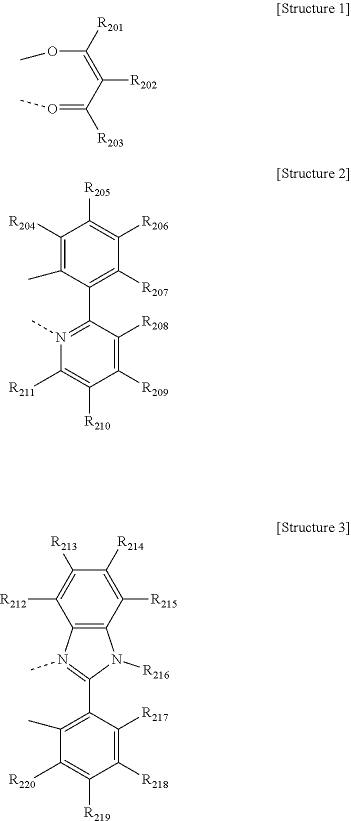

R₁₀₀ to R₁₀₃ each independently represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent substituent to form a ring(s), e.g., a substituted or unsubstituted, quinoline, benzofuropyridine, benzothienopyridine, indenopyridine, benzofuroquinoline, benzothienoquinoline, or indenoquinoline, together with a pyridine;
R₁₀₄ to R₁₀₇ each independently represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent to form a substituted or unsubstituted ring(s), e.g., a substituted or unsubstituted, naphthalene, fluorene, dibenzothiophene, dibenzofuran, indenopyridine, benzofuropyridine, or benzothienopyridine, together with a benzene;
R₂₀₁ to R₂₂₀ each independently represent hydrogen, deuterium, a halogen, a (C1-C30)alkyl unsubstituted or substituted with deuterium and/or a halogen(s), a substituted or unsubstituted (C3-C30)cycloalkyl, or a substituted or unsubstituted (C6-C30)aryl; or may be linked to an adjacent substituent to form a substituted or unsubstituted ring(s); and
s represents an integer of 1 to 3.

The specific examples of the dopant compound are as follows, but are not limited thereto.

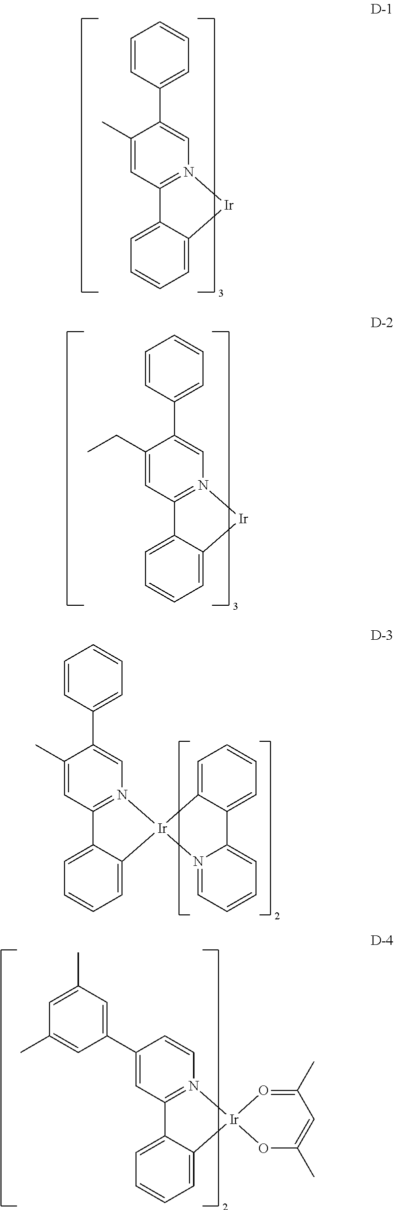

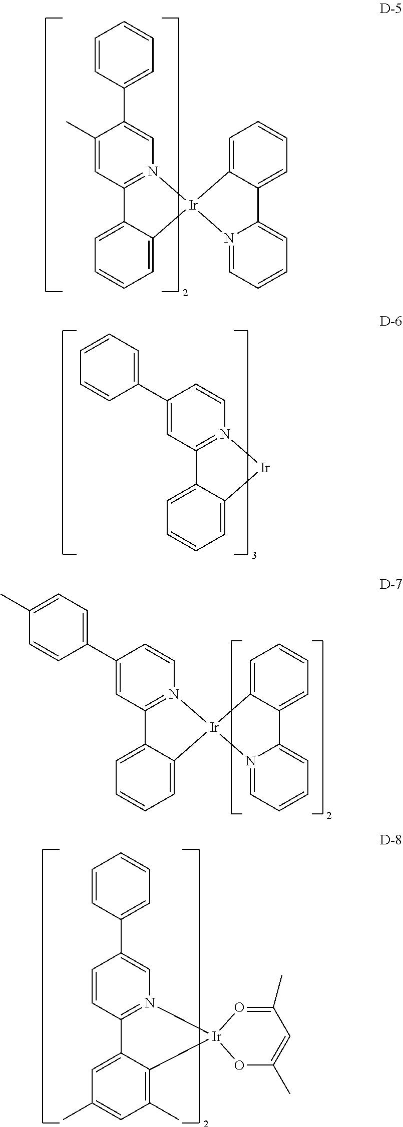

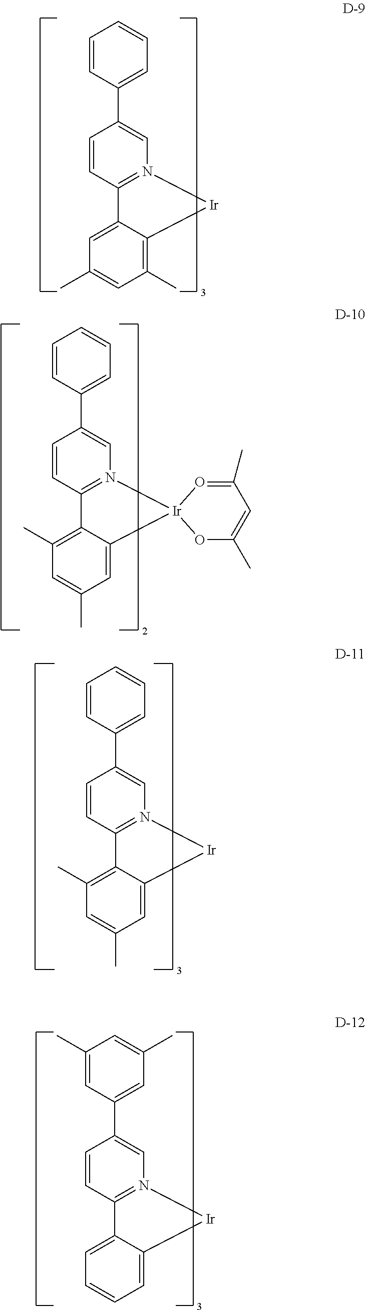

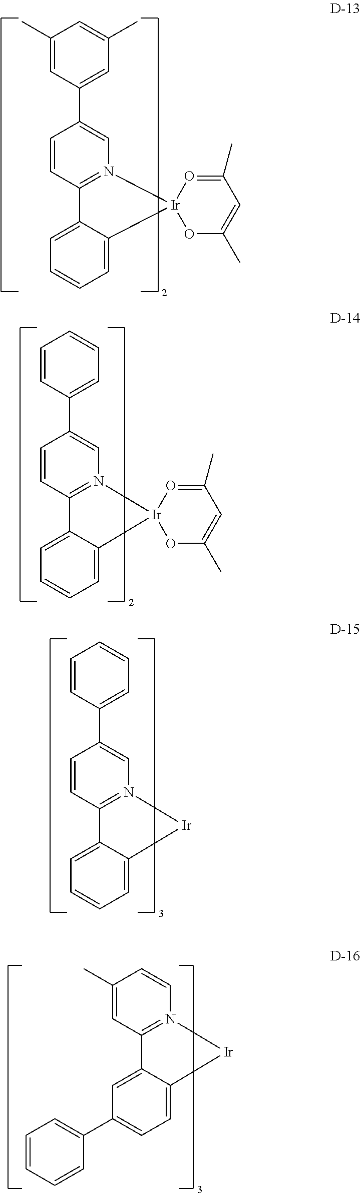

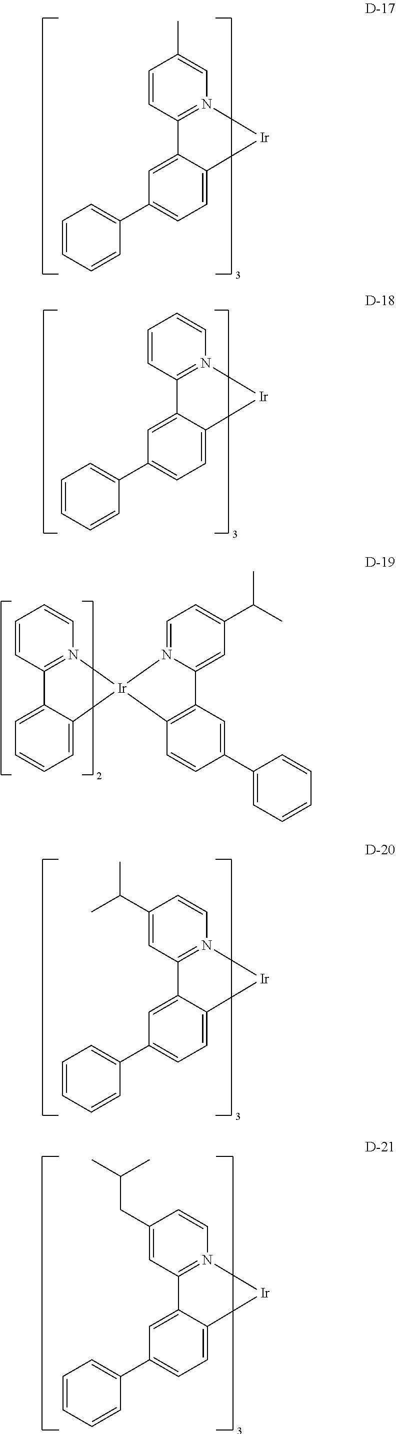

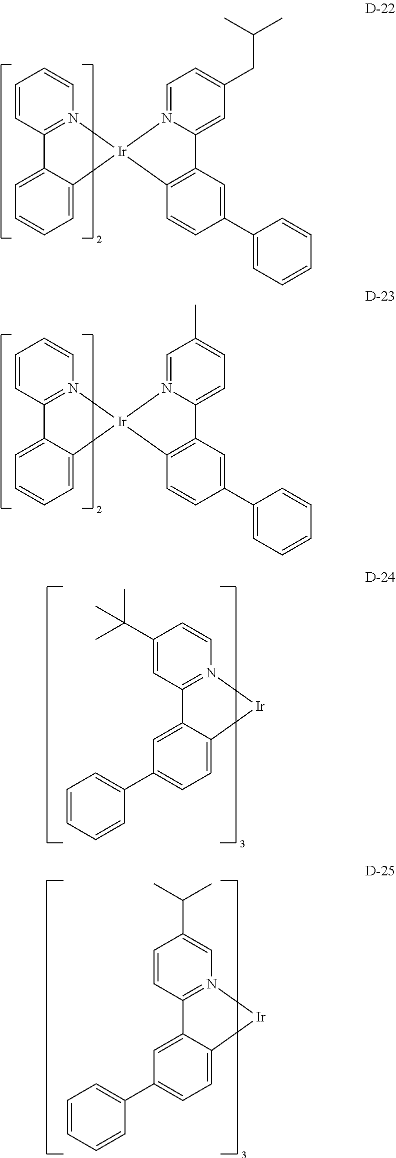

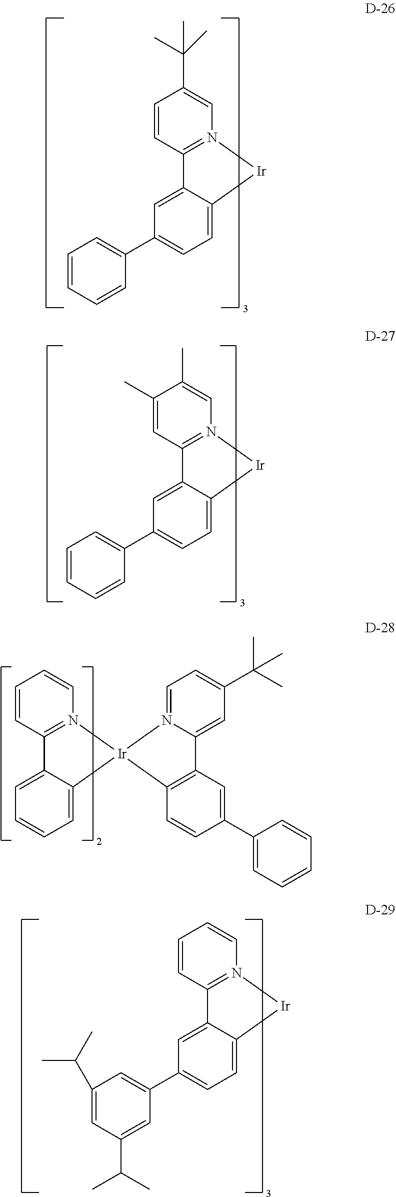

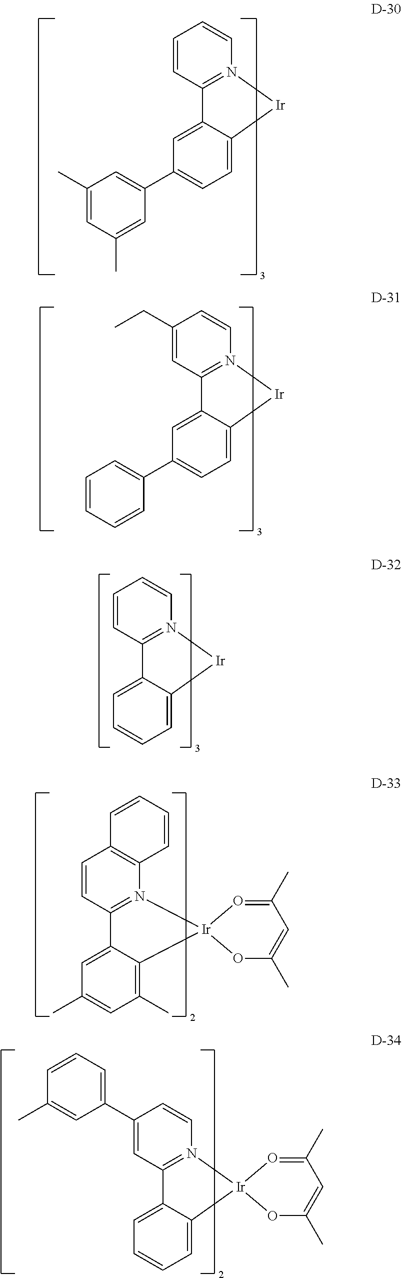

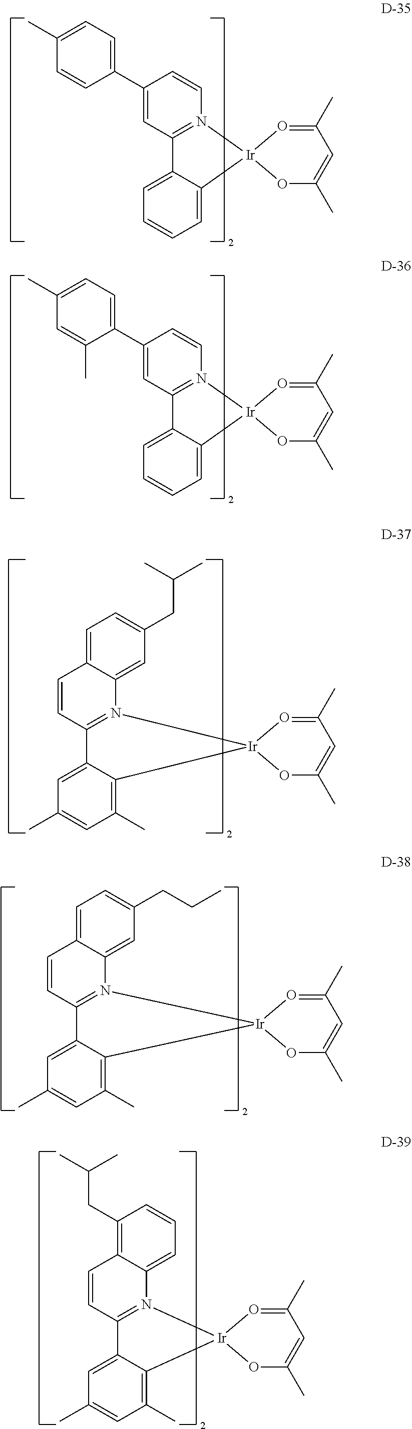

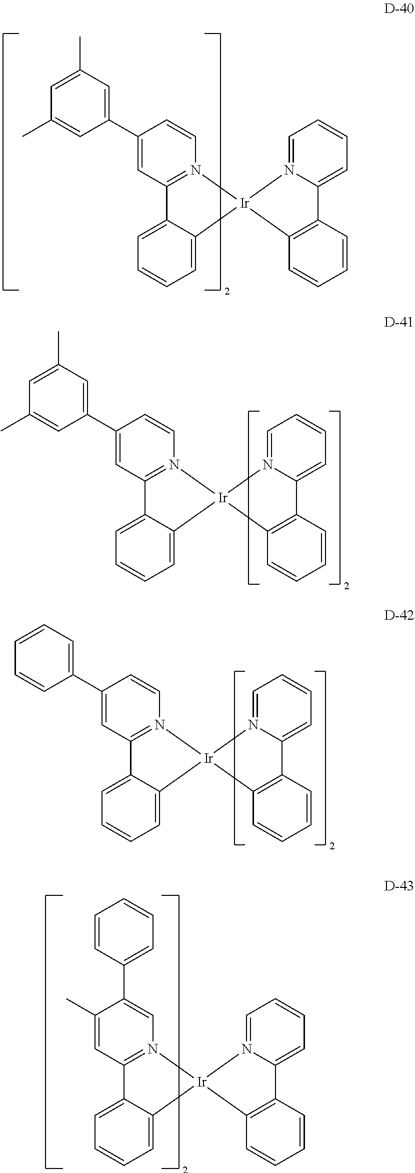

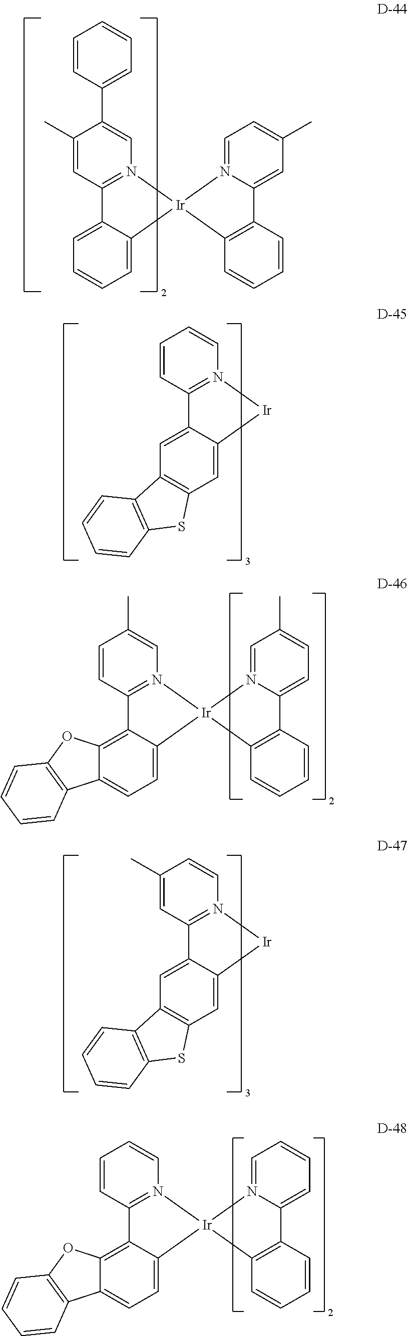

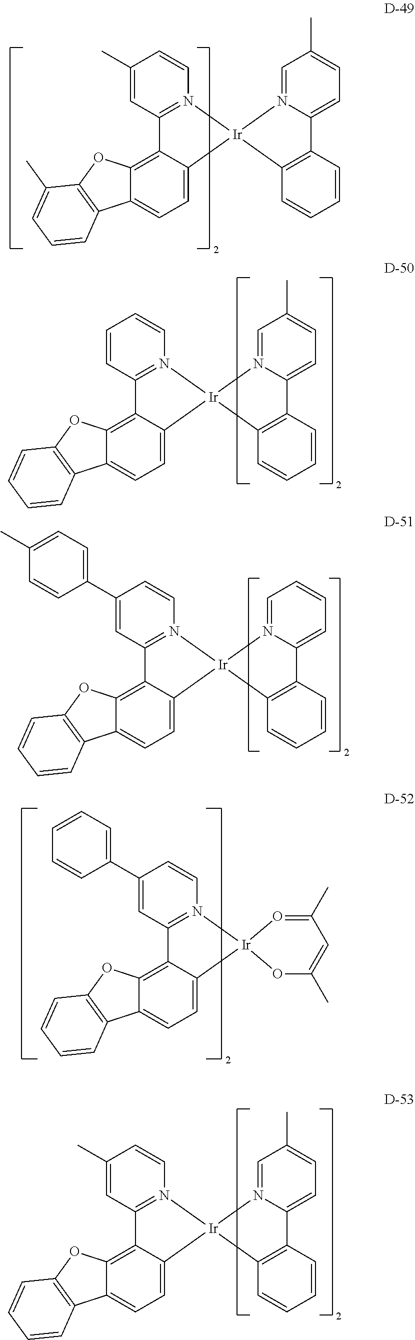

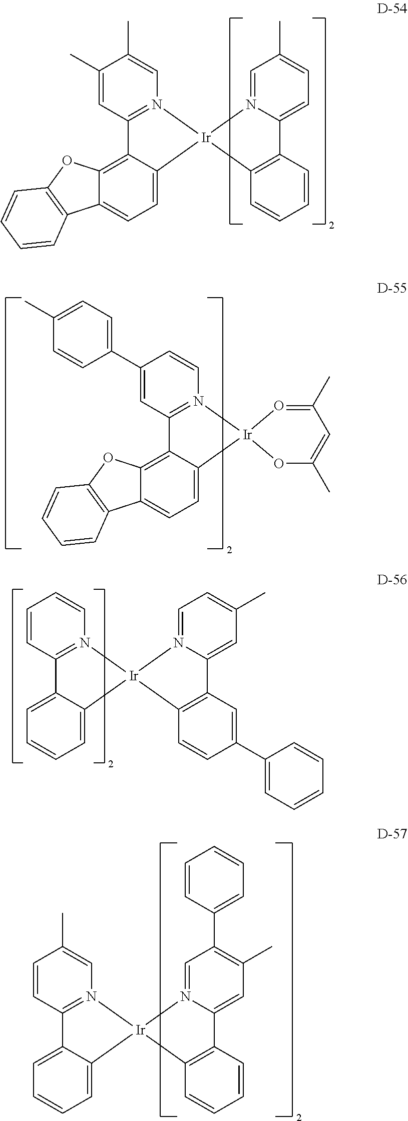

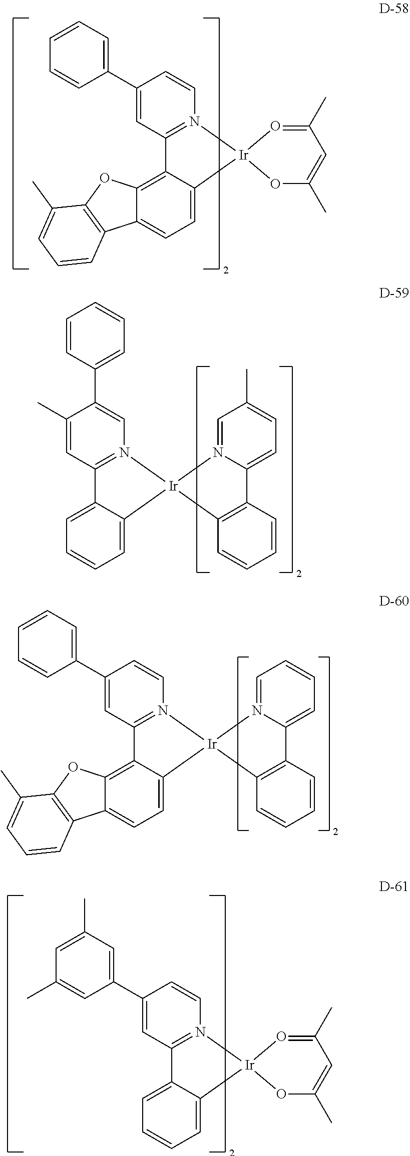

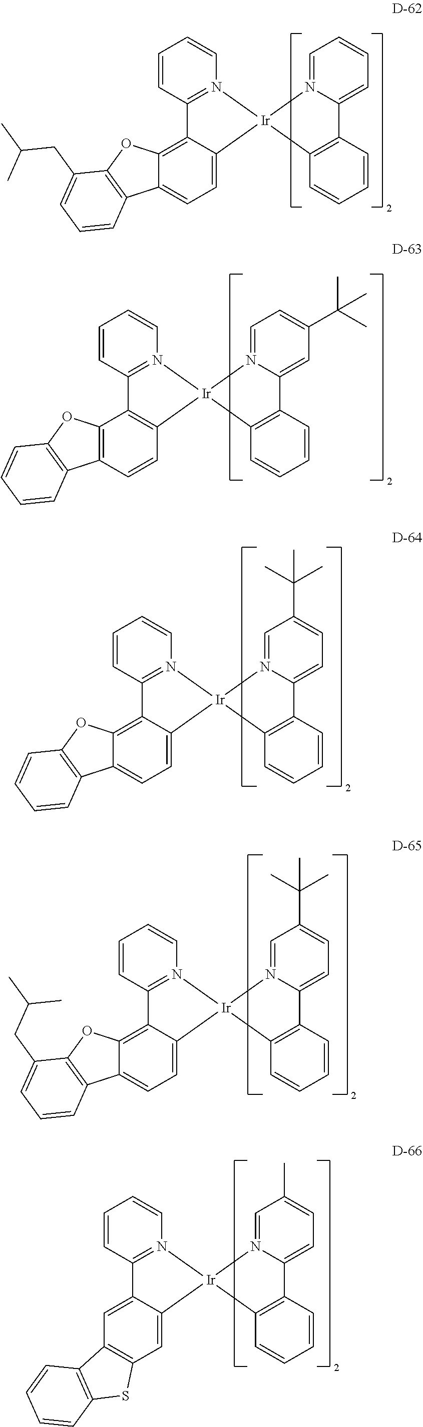

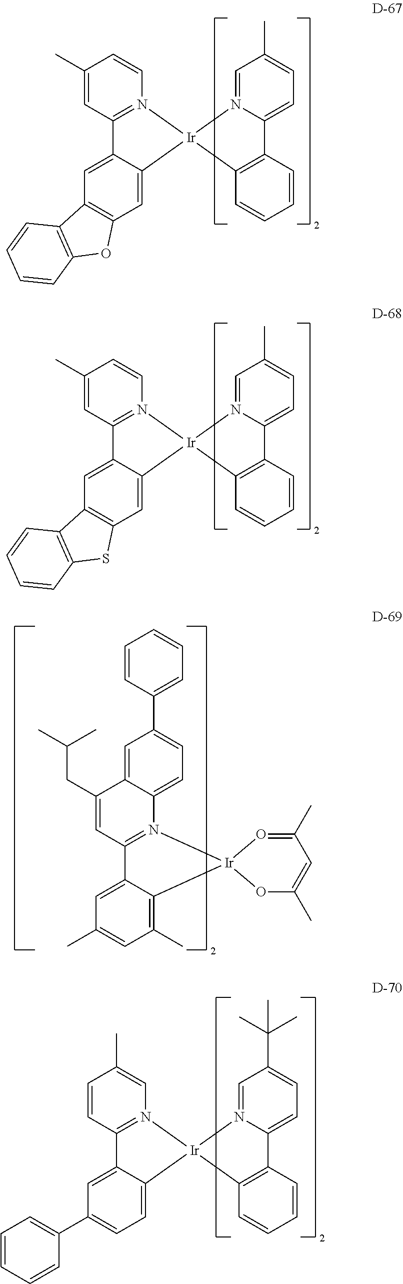

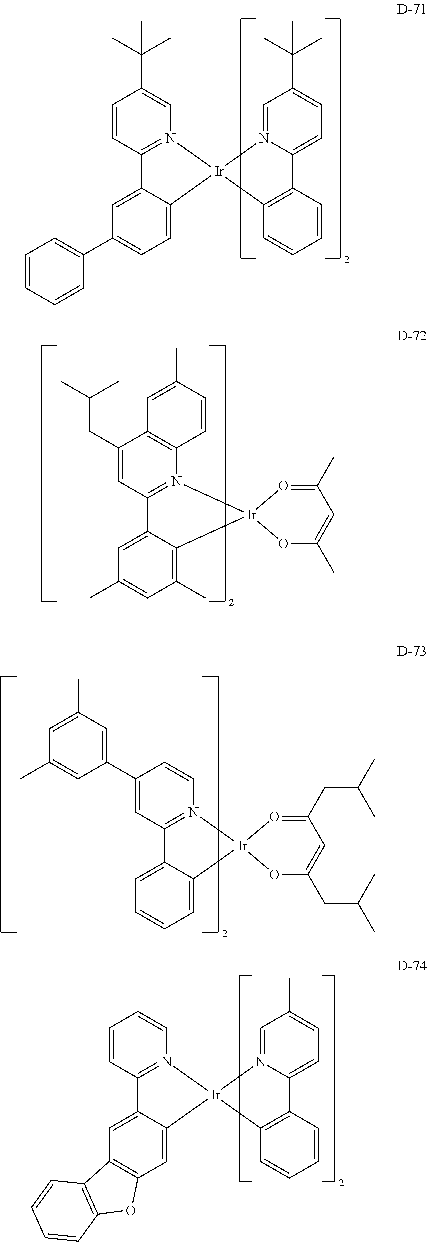

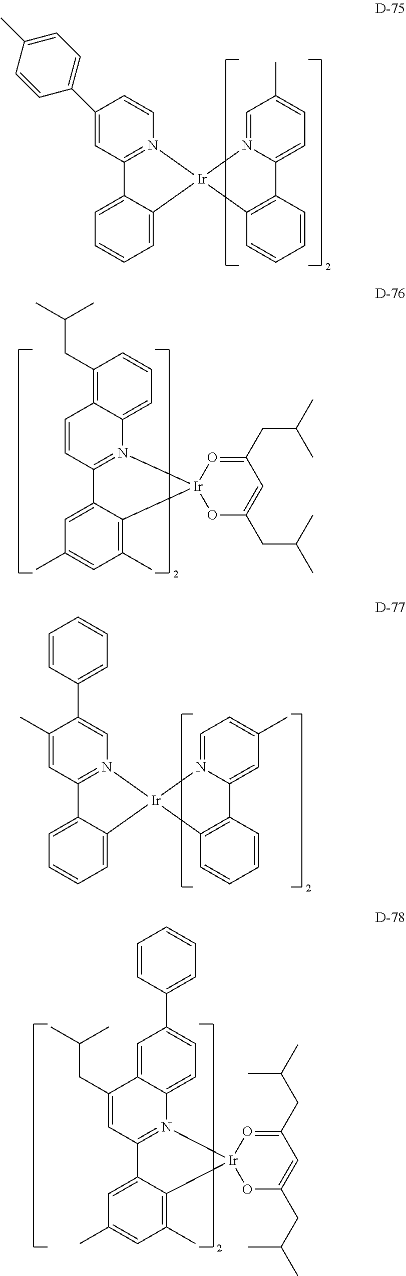

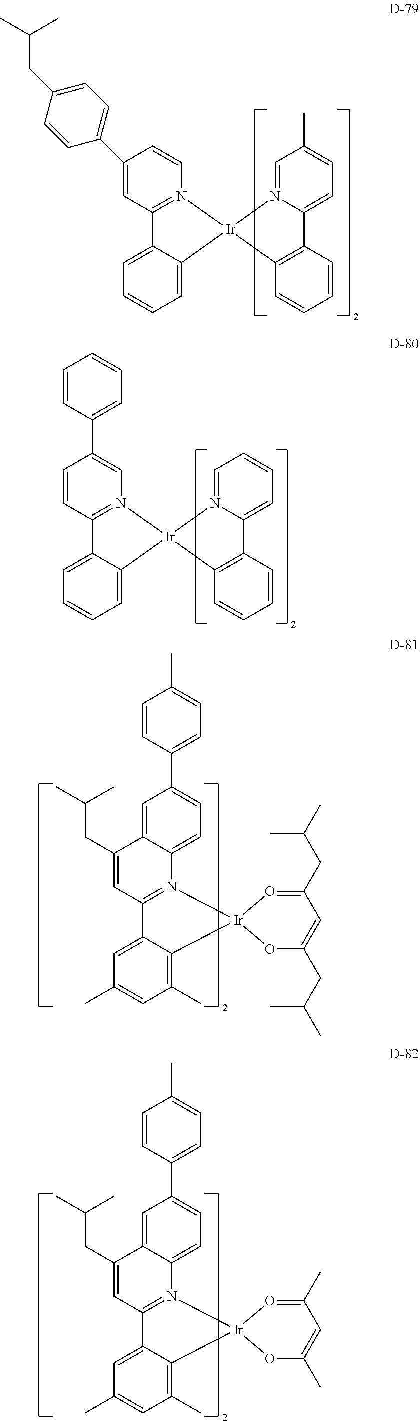

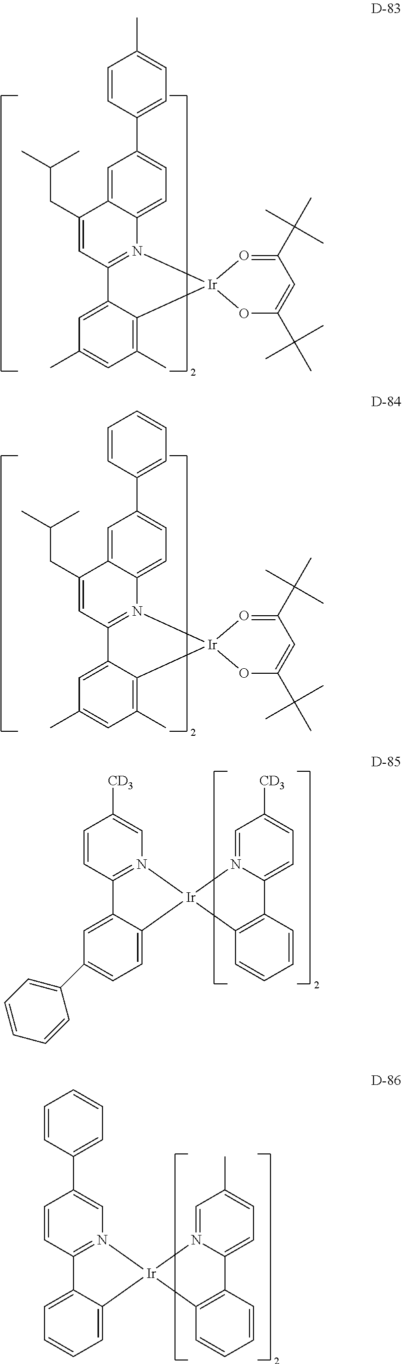

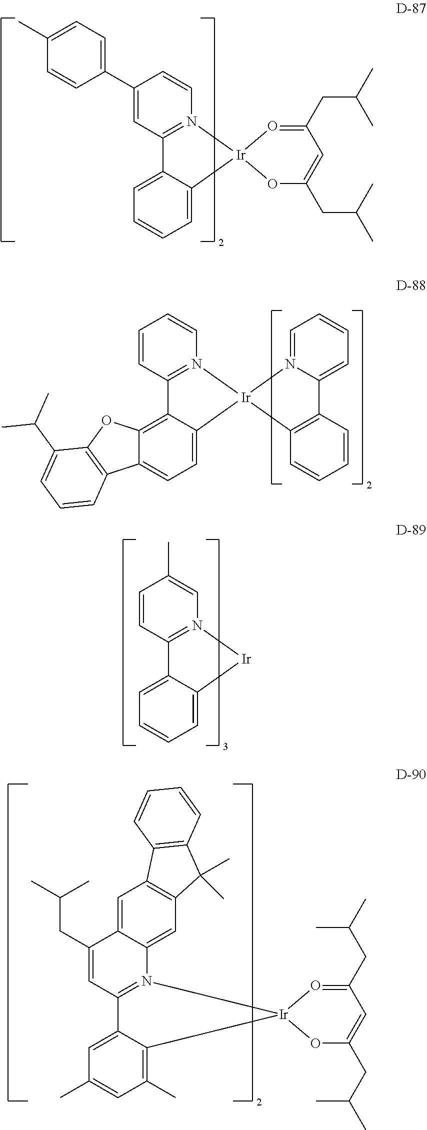

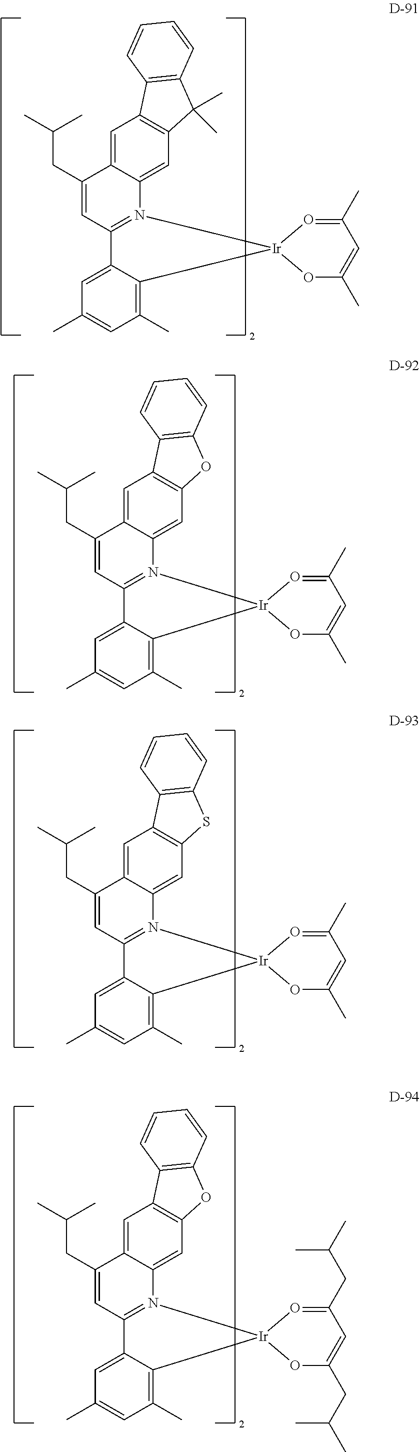

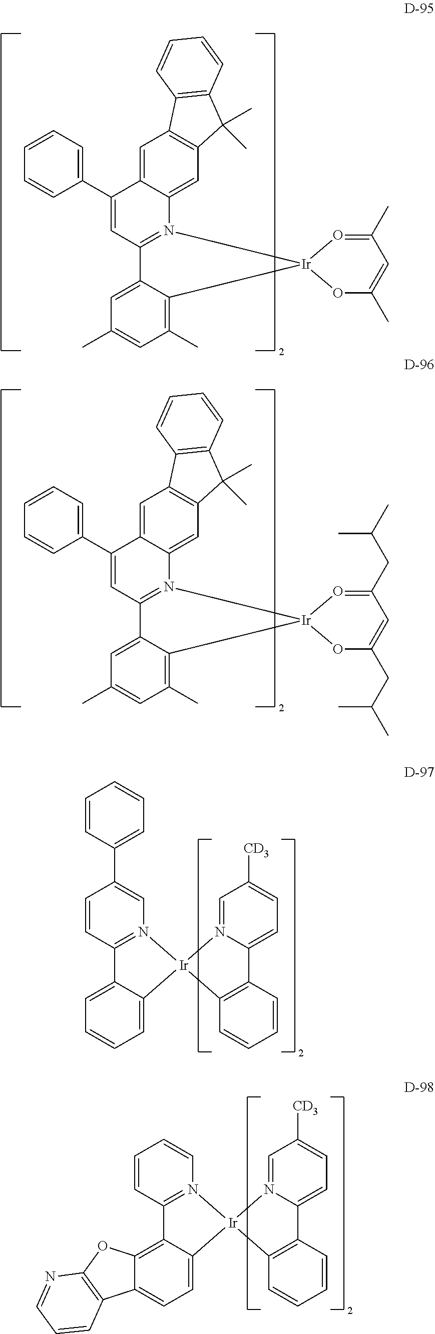

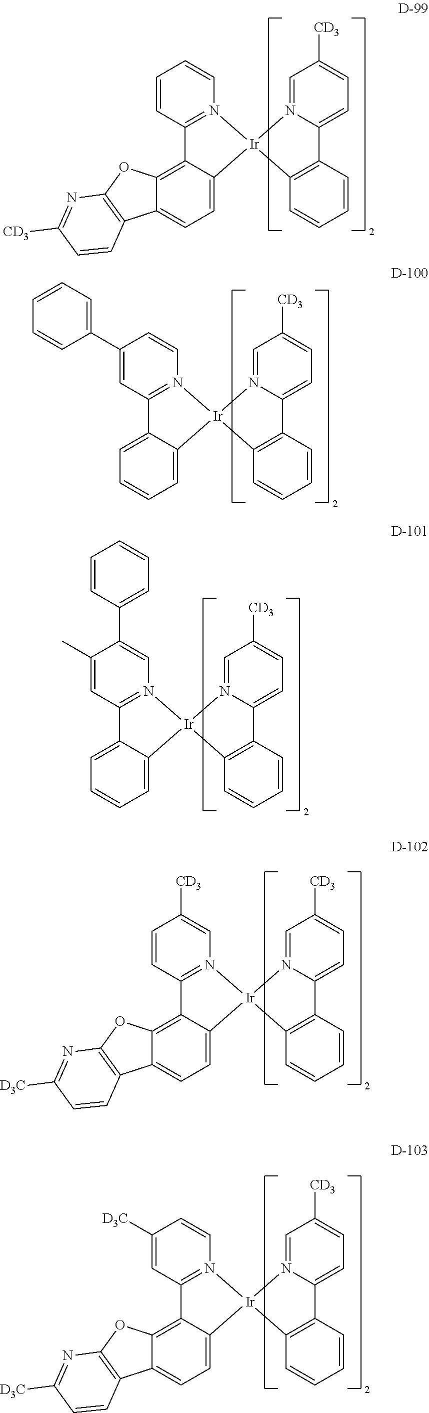

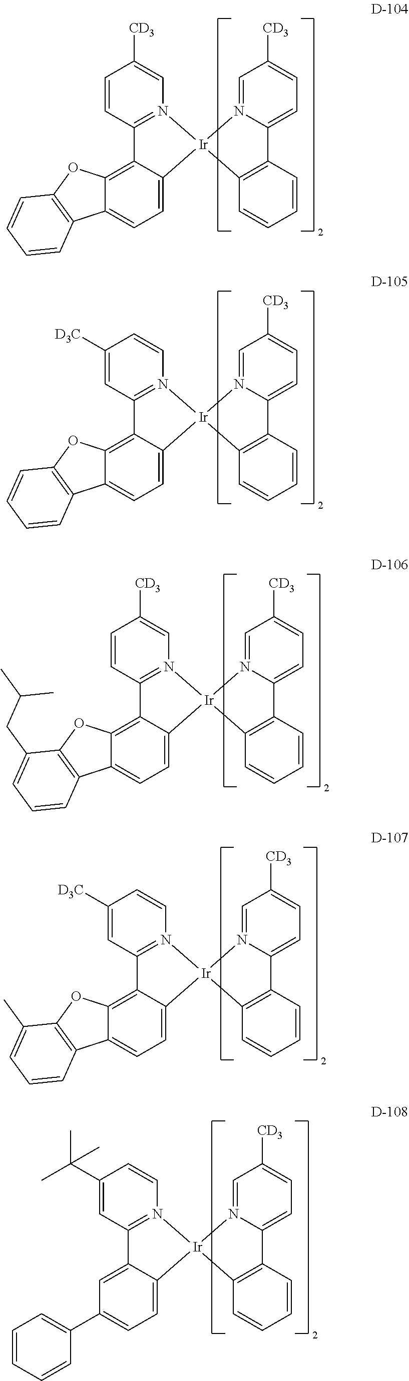

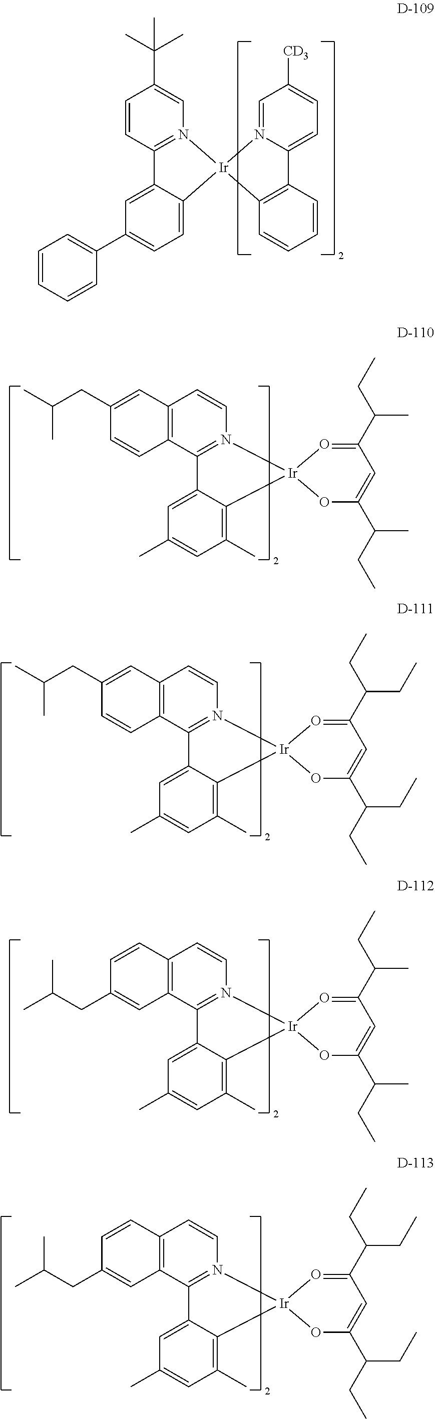

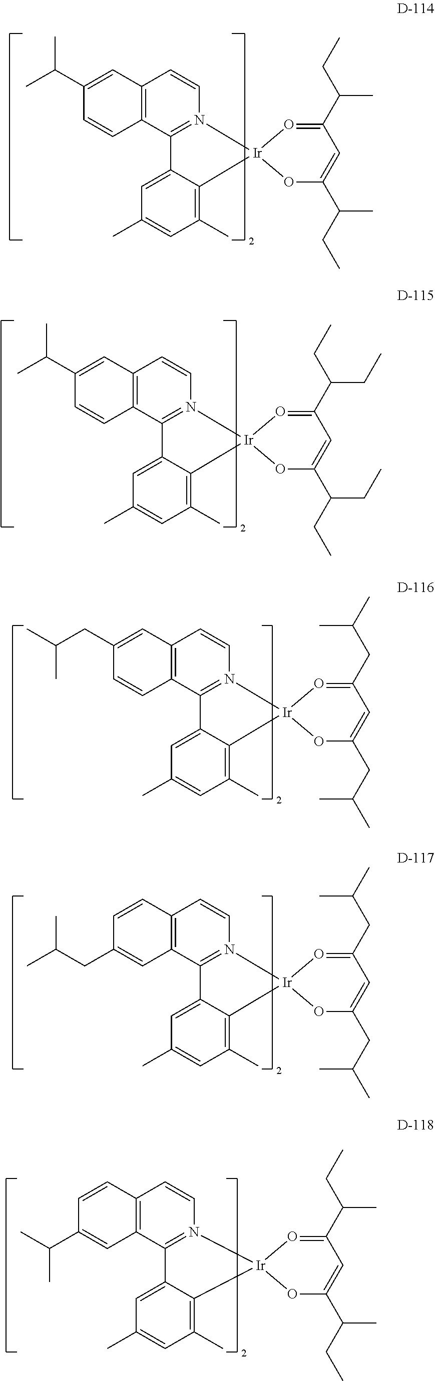

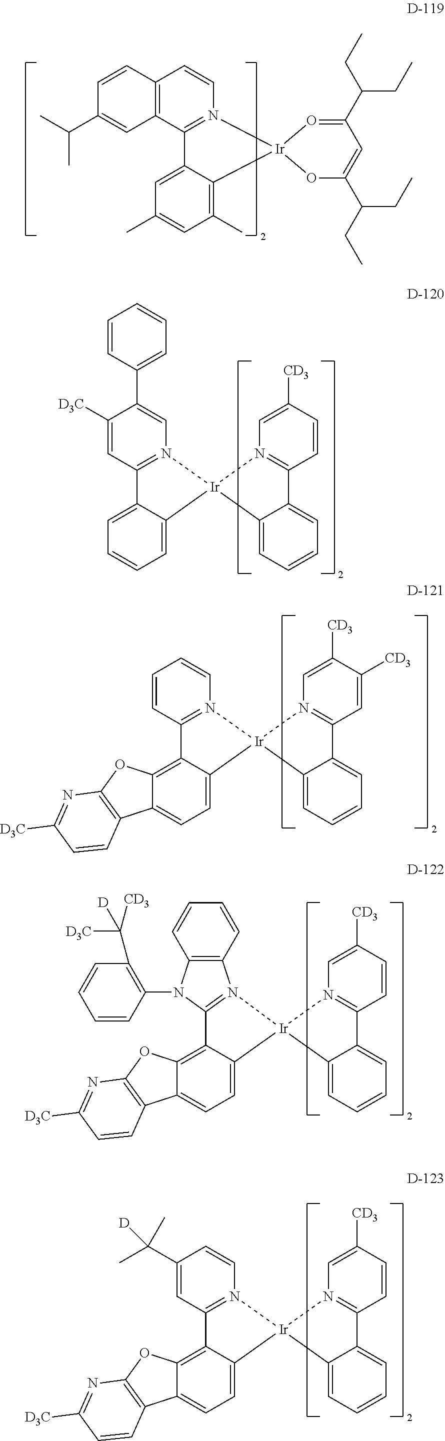

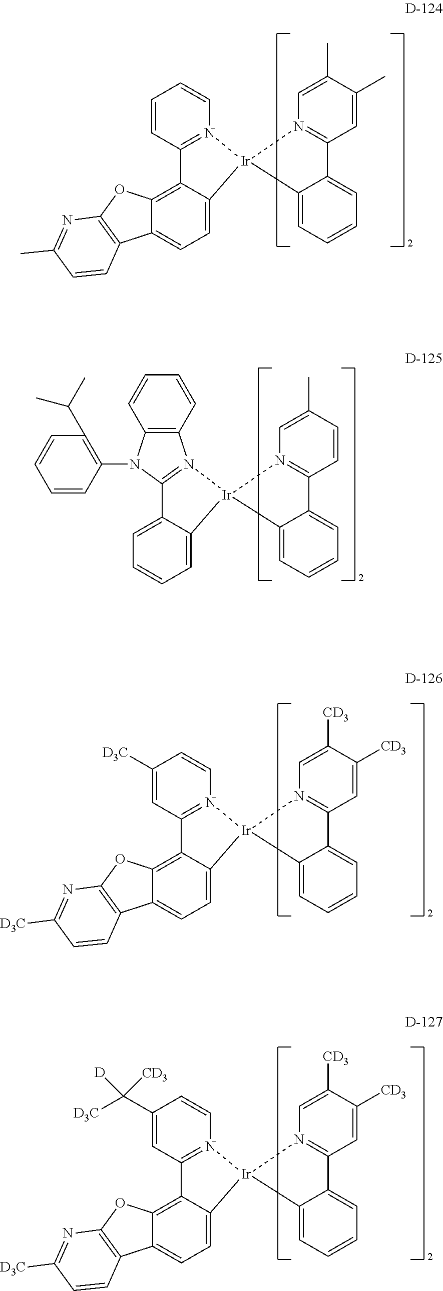

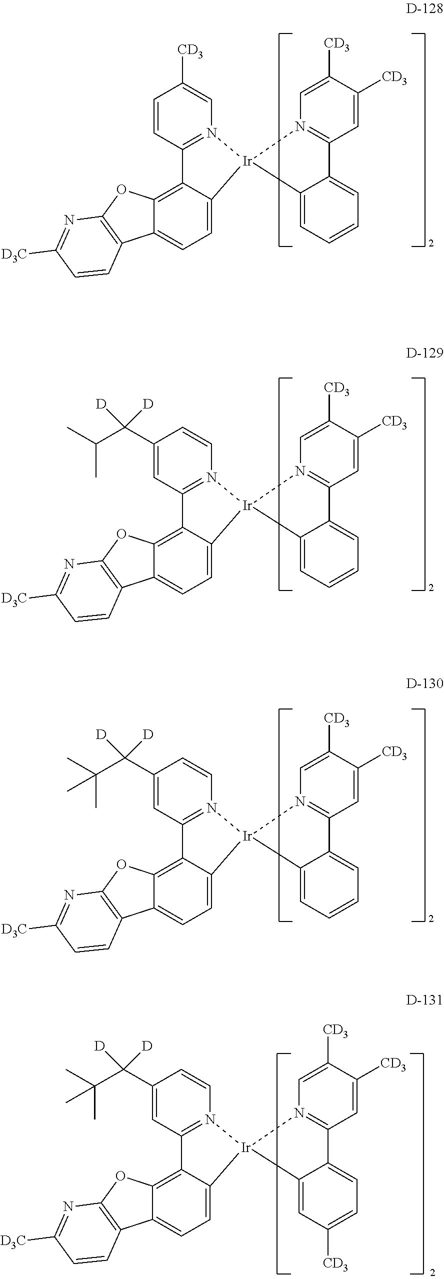

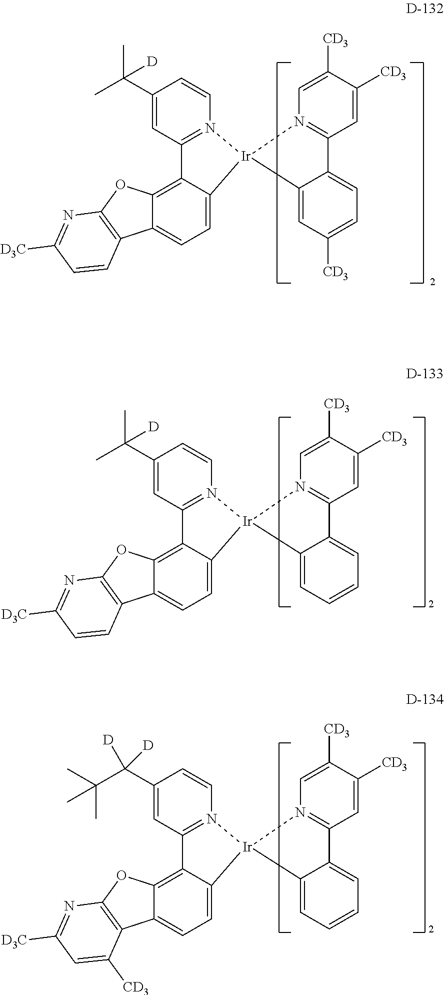

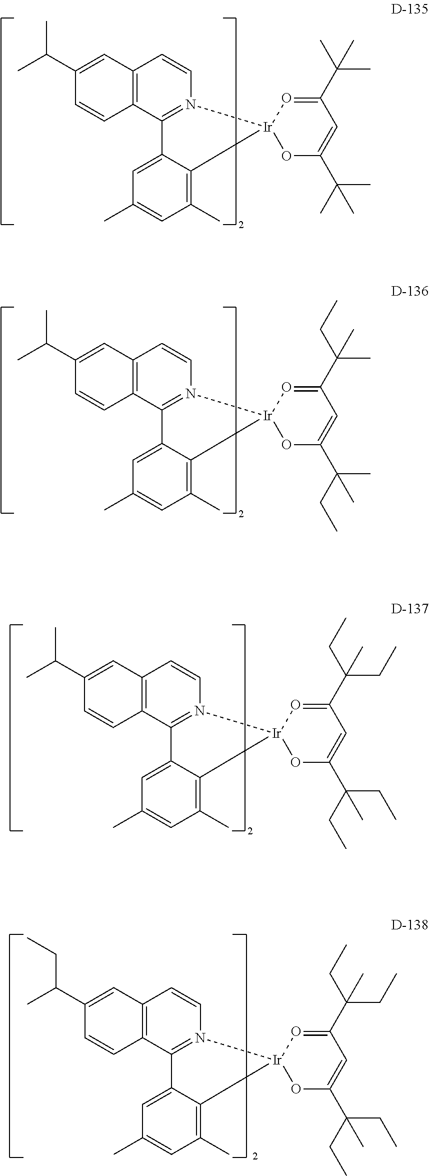

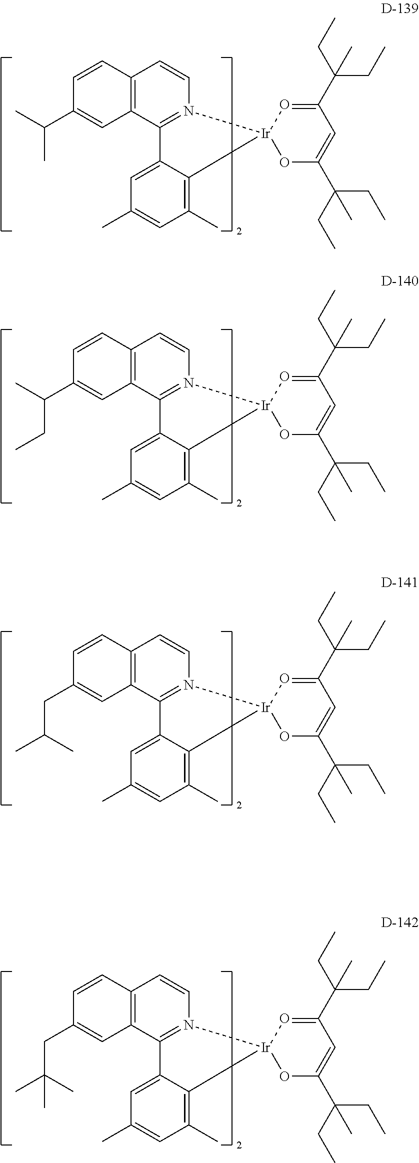

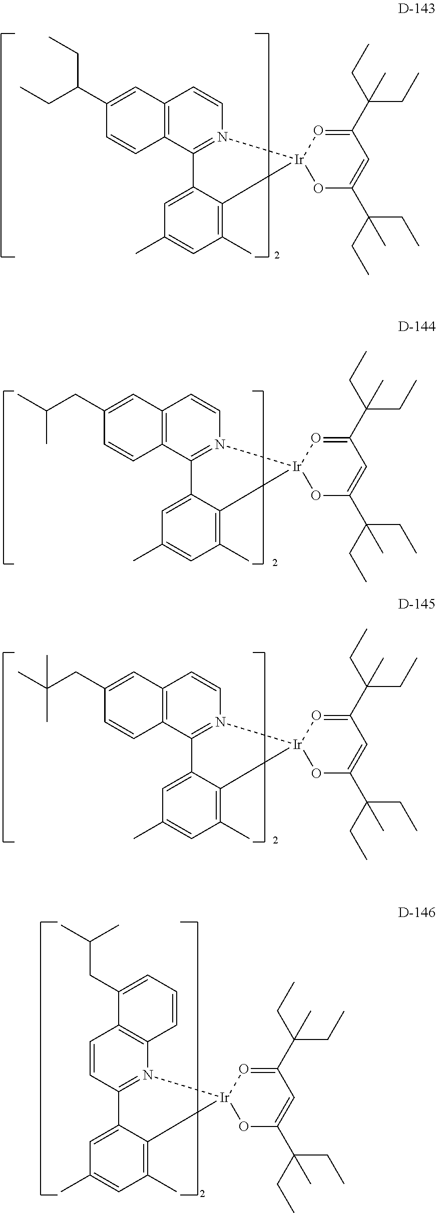

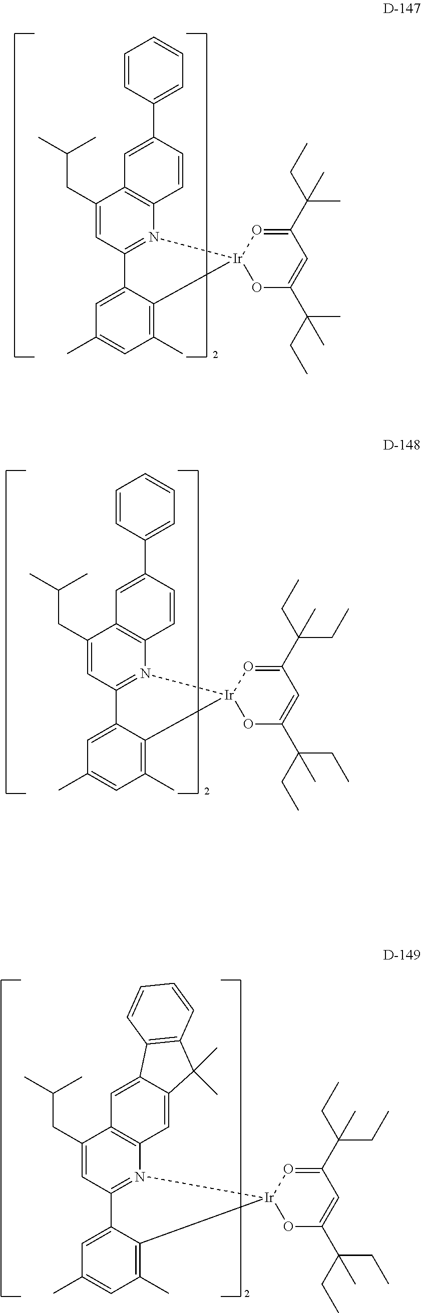

The organic electroluminescent device according to the present disclosure comprises an anode; a cathode; and at least one organic layer between the anode and the cathode. The organic layer comprises a light-emitting layer, and may further comprise at least one layer selected from any of the following: a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer. Each of the layers may be further configured as a plurality of layers.

Each of the anode and the cathode may be formed of a transparent conductive material or a transflective or reflective conductive material. Depending on the type of material forming the anode and the cathode, the organic electroluminescent device may be a top light-emitting type, a bottom light-emitting type, or a double side light-emitting type. In addition, the hole injection layer may be further doped with a p-dopant(s), and the electron injection layer may be further doped with an n-dopant(s).

At least one compound(s) selected from the group consisting of arylamine-based compounds and styrylarylamine-based compounds may be further comprised in the organic layer. In addition, the organic material layer may further comprise at least one metal selected from the group consisting of metals of Group 1, metals of Group 2, transition metals of the 4th period, transition metals of the 5th period, lanthanides, and organic metals of the d-transition elements of the Periodic Table, or at least one complex compound comprising the metal.

In addition, the organic electroluminescent device of the present disclosure may emit white light by further comprising at least one light-emitting layer, which comprises a blue, red, or green light-emitting compound known in the art, besides the compound of the present disclosure. In addition, if necessary, it may further comprise a yellow or orange light-emitting layer.

In the organic electroluminescent device of the present disclosure, it is preferable to dispose at least one layer selected from a chalcogenide layer, a metal halide layer, and a metal oxide layer (hereinafter referred to as a “surface layer”) on at least one inner surface of a pair of electrodes. Specifically, a chalcogenide (including an oxide) layer of silicon and aluminum is preferably placed on an anode surface of an electroluminescent medium layer side, and a metal halide layer or a metal oxide layer is preferably placed on a cathode surface of an electroluminescent medium layer side. Driving stabilization of the organic electroluminescent device can be obtained by the surface layer. Preferred examples of the chalcogenide include SiO_{X}(1≤X≤2), AlO_{X}(1≤X≤1.5), SiON, SiAlON, etc., preferred examples of the metal halide include LiF, MgF₂, CaF₂, a rare earth metal fluoride, etc., and preferred examples of the metal oxide include Cs₂O, Li₂O, MgO, SrO, BaO, CaO, etc.

A hole injection layer, a hole transport layer, an electron-blocking layer, or a combination thereof may be used between an anode and a light-emitting layer. The hole injection layer may be multi-layered in order to lower the hole injection barrier (or hole injection voltage) from the anode to the hole transport layer or the electron-blocking layer, wherein two compounds may be simultaneously used in each of the multi-layers. The hole transport layer or the electron-blocking layer may be multi-layered.

An electron buffer layer, a hole-blocking layer, an electron transport layer, an electron injection layer, or a combination thereof may be used between a light-emitting layer and a cathode. The electron buffer layer may be multi-layered in order to control electron injection and improve interfacial properties between the light-emitting layer and the electron injection layer, wherein two compounds may be simultaneously used in each of the multi-layers. The hole-blocking layer or the electron transport layer may be multi-layered, wherein a plurality of compounds may be used in each of the multi-layers.

A light-emitting auxiliary layer may be a layer placed between an anode and a light-emitting layer, or between a cathode and a light-emitting layer. When placed between the anode and the light-emitting layer, the light-emitting auxiliary layer may be used to facilitate hole injection and/or hole transport or to block the overflow of electrons. When placed between the cathode and the light-emitting layer, the light-emitting auxiliary layer may be used to facilitate electron injection and/or electron transport or to block the overflow of holes. In addition, the hole auxiliary layer may be placed between the hole transport layer (or hole injection layer) and the light-emitting layer, and may exhibit an effect of facilitating or blocking the hole transport rate (or hole injection rate), and accordingly may adjust the charge balance. In addition, the electron-blocking layer may be placed between the hole transport layer (or hole injection layer) and the light-emitting layer, and may block the overflow of electrons from the light-emitting layer and confine the excitons in the light-emitting layer to prevent light leakage. When an organic electroluminescent device includes two or more hole transport layers, the hole transport layer, which is further included, may be used as a hole auxiliary layer or an electron-blocking layer. The light-emitting auxiliary layer, the hole auxiliary layer, or the electron-blocking layer may have an effect of improving the efficiency and/or lifetime of the organic electroluminescent device.

In addition, in an organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to the light-emitting medium. Furthermore, the hole transport compound is oxidized to a cation, and thus it becomes easier to inject and transport holes from the mixed region to the light-emitting medium. Preferred oxidative dopants include various Lewis acids and acceptor compounds, and preferred reductive dopants include alkali metals, alkali metal compounds, alkaline earth metals, rare earth metals, and mixtures thereof. In addition, an organic electroluminescent device having at least two light-emitting layers and emitting white light may be manufactured by using the reductive dopant layer as a charge generation layer.

An organic electroluminescent device according to the present disclosure may be an organic electroluminescent device having a tandem structure. In the case of the tandem organic electroluminescent device according to one embodiment, a single light-emitting unit (light-emitting part) may be formed in a structure in which two or more units are connected by a charge generation layer. The organic electroluminescent device may include a plurality of two or more light-emitting units, for example, a plurality of three or more light-emitting units, having first and second electrodes opposed to each other on a substrate and a light-emitting layer stacked between the first and second electrodes and emits light in a specific wavelength range. It may include a plurality of light-emitting units, and each of the light-emitting units may include a hole transport zone, a light-emitting layer, and an electron transport zone, the hole transport zone may include a hole injection layer and a hole transport layer, and the electron transport zone may include an electron transport layer and an electron injection layer. According to one embodiment of the present disclosure, three or more light emitting layers may be included in the light emitting unit. A plurality of light-emitting units may emit the same color or different colors. Additionally, one light-emitting unit may include one or more light-emitting layers, and the plurality of light-emitting layers may be light-emitting layers of the same or different colors. It may include one or more charge-generation layers located between each light-emitting unit. The charge-generation layer refers to the layer in which holes and electrons are generated when voltage is applied. When there are three or more light-emitting units, a charge-generation layer may be located between each light-emitting unit. At this time, the plurality of charge generation layers may be the same as or different from each other. By disposing the charge generating layer between light-emitting units, current efficiency is increased in each light-emitting unit and charges can be smoothly distributed. Specifically, the charge generation layer is provided between two adjacent stacks and can serve to drive a tandem organic electroluminescent device using only a pair of anodes and cathodes without a separate internal electrode located between the stacks.

The charge generation layer may be composed of an n-type charge generation layer and a p-type charge generation layer, and the n-type charge generation layer may be doped with an alkali metal, an alkaline earth metal, or a compound of an alkali metal and an alkaline earth metal. The alkali metal may include one selected from the group consisting of Li, Na, K, Rb, Cs, Fr, Yb, and combinations thereof, and the alkaline earth metal may include one selected from the group consisting of Be, Mg, Ca, Sr, Ba, Ra, and combinations thereof. The p-type charge generation layer may be made of a metal or an organic material doped with a p-type dopant. For example, the metal may be made of one or two or more alloys selected from the group consisting of Al, Cu, Fe, Pb, Zn, Au, Pt, W, In, Mo, Ni, and Ti. Additionally, commonly used materials may be used as the p-type dopant and host materials used in the p-type doped organic material.

The organic electroluminescent material according to one embodiment of the present disclosure may be used as a light-emitting material for a white organic light-emitting device. The white organic light-emitting device has been suggested to have various structures such as a side-by-side arrangement method, a stacking arrangement method, or a color conversion material (CCM) method, etc., according to the arrangement of R (Red), G (Green) or YG (Yellowish Green), and B (Blue) light-emitting units. In addition, the organic electroluminescent material according to one embodiment of the present disclosure may also be used in an organic electroluminescent device comprising a quantum dot (QD).

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used. When forming a film of the first host compound and the second host compound of the present disclosure, co-deposition or mixed deposition is performed.

When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

In addition, it is possible to produce a display system, e.g., a display system for smartphones, tablets, notebooks, PCs, TVs, or cars; or a lighting system, e.g., an outdoor or indoor lighting system, using the organic electroluminescent device of the present disclosure.

Hereinafter, the preparation method of the compound of the present disclosure, the properties thereof, and the driving voltage and the luminous efficiency of the organic electroluminescent device (OLED) comprising an organic electroluminescent compound according to the present disclosure will be explained in detail with reference to the representative compounds of the present disclosure. However, the following examples only describe the properties of the compound according to the present disclosure and the OLED comprising the same, and the present disclosure is not limited to the following examples.

Hereinafter, the preparation method of the host materials according to the present disclosure will be explained with reference to the synthesis method of a representative compound or intermediate compound in order to understand the present disclosure in detail.

### Example 1: Preparation of Compound C-86

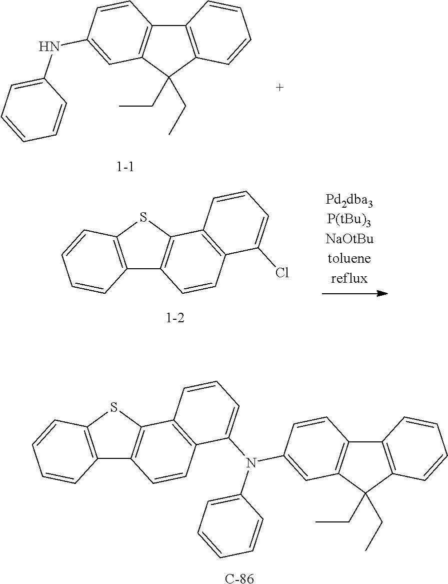

In a flask, compound 1-1 (CAS: 373390-079) (10.5 g, 33.5 mmol), compound 1-2 (CAS: 2408008-08-0) (10.0 g, 37.2 mmol), tris(dibenzylidenacetone)dipalladium(0) (1.70 g, 1.86 mmol), tri-t-butylphosphine (1.84 mL, 3.72 mmol, 50% toluene solution), sodium t-butoxide (7.15 g, 74.4 mmol), and 186 mL of toluene were added and refluxed for 2 hours. The reaction solution was cooled to room temperature, and the solvent was removed by using a rotary evaporator, and it was then purified by column chromatography to obtain a white solid compound, C-86 (11.5 g, yield: 57%).

### Example 2: Preparation of Compound C-111

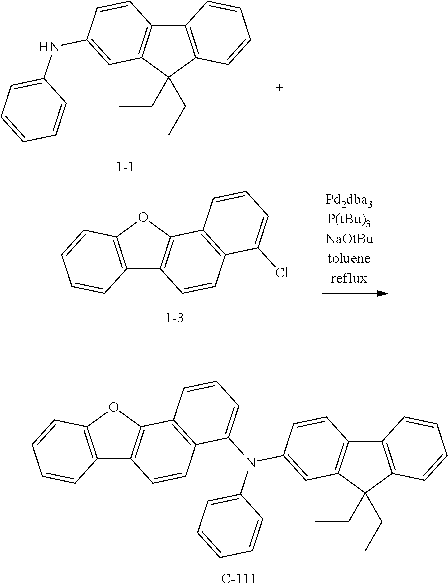

In a flask, compound 1-1 (CAS: 373390-079) (11.2 g, 35.6 mmol), compound 1-3 (CAS: 2305687-93-6) (10.0 g, 39.6 mmol), tris(dibenzylidenacetone)dipalladium(0) (1.81 g, 1.98 mmol), tri-t-butylphosphine (1.95 mL, 3.96 mmol, 50% toluene solution), sodium t-butoxide (7.61 g, 79.1 mmol), and 198 mL of toluene were added and refluxed for 2 hours. The reaction solution was cooled to room temperature, and the solvent was removed by using a rotary evaporator, and it was then purified by column chromatography to obtain a white solid compound, C-111 (5.2 g, yield: 28%).

### Example 3: Preparation of Compound C-1

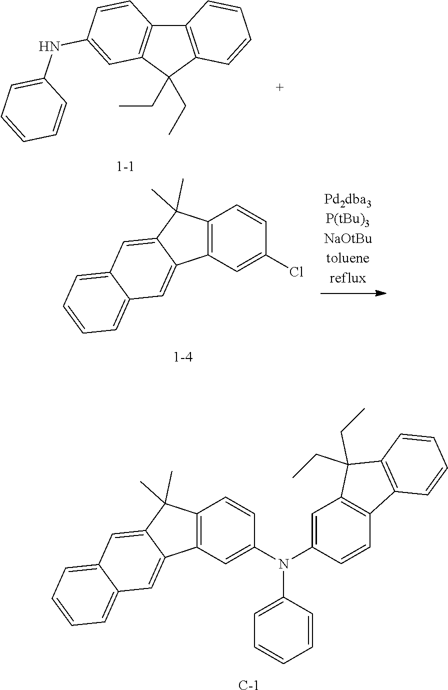

In a flask, compound 1-1 (CAS: 373390-079) (10.0 g, 32.0 mmol), compound 1-4 (CAS: 2397634-76-1) (9.7 g, 35.0 mmol), tris(dibenzylidenacetone)dipalladium(0) (1.5 g, 1.60 mmol), tri-t-butylphosphine (1.58 mL, 3.20 mmol, 50% toluene solution), sodium t-butoxide (6.15 g, 64.0 mmol), and 160 mL of toluene were added and refluxed for 1 hour. The reaction solution was cooled to room temperature, and the solvent was removed by using a rotary evaporator, and it was then purified by column chromatography to obtain a white solid compound, C-1 (8.9 g, yield: 50%).

### Example 4: Preparation of Compound C-36

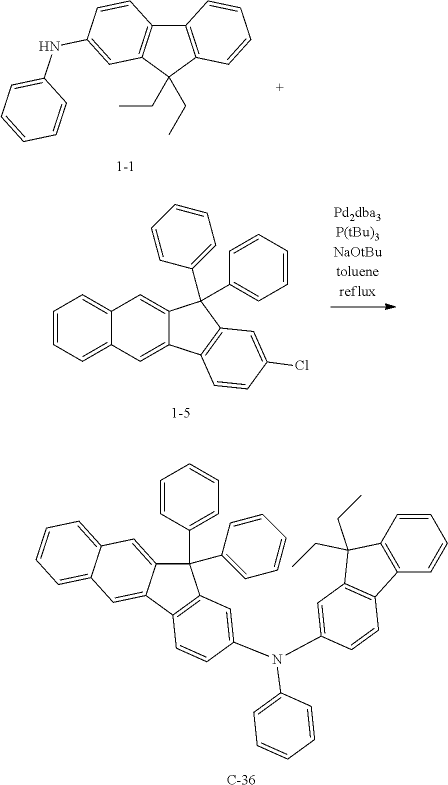

In a flask, compound 1-1 (CAS: 373390-079) (8.0 g, 26.0 mmol), compound 1-5 (CAS: 2412522-09-7) (12.3 g, 31.0 mmol), tris(dibenzylidenacetone)dipalladium(0) (1.20 g, 1.30 mmol), tri-t-butylphosphine (1.30 mL, 2.60 mmol, 50% toluene solution), sodium t-butoxide (4.9 g, 52.0 mmol), and 130 mL of toluene were added and refluxed for 1 hour. The reaction solution was cooled to room temperature, and the solvent was removed by using a rotary evaporator, and it was then purified by column chromatography to obtain a white solid compound, C-36 (15.4 g, yield: 89%).

| | HOMO | LUMO | Triplet Energy | MP (° C.) | Tg (° C.) |
|---|---|---|---|---|---|
| C-86 | −4.900 | −1.325 | 2.427 | 265 | 100 |
| C-111 | −4.883 | −1.198 | 2.481 | 204 | 90 |
| C-1 | −4.809 | −1.230 | 2.503 | 322 | 106 |
| C-36 | −4.775 | −1.257 | 2.369 | 266 | 127 |

### Example 5: Preparation of Compound C-279

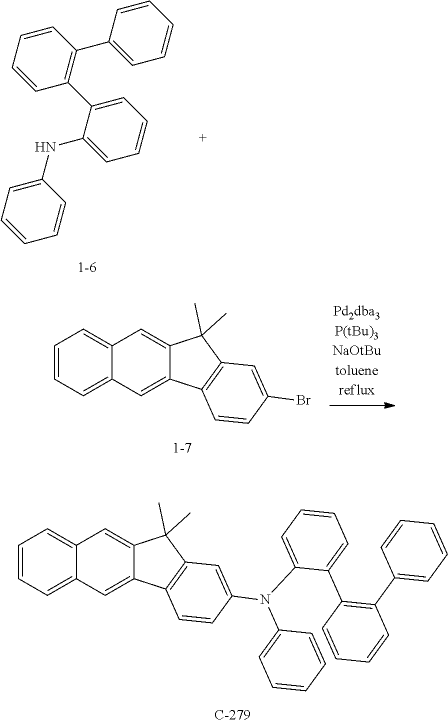 [SMILES: c1ccc(Nc2ccccc2-c2ccccc2-c2ccccc2)cc1 ; CC1(C)c2cc(Br)ccc2-c2cc3ccccc3cc21 ; CC1(C)c2cc(N(c3ccccc3)c3ccccc3-c3ccccc3-c3ccccc3)ccc2-c2cc3ccccc3cc21]

In a flask, compound 1-6 (CAS: 1776936-11-8) (3.0 g, 9.33 mmol), compound 1-7 (CAS: 1198396-39-2) (4.2 g, 13.1 mmol), tris(dibenzylidenacetone)dipalladium(0) (0.43 g, 0.47 mmol), tri-t-butylphosphine (0.45 mL, 0.93 mmol, 50% toluene solution), sodium t-butoxide (1.35 g, 14.0 mmol), and 48 mL of toluene were added and refluxed for 1 hour. The reaction solution was cooled to room temperature, and the solvent was removed by using a rotary evaporator, and it was then purified by column chromatography to obtain a white solid compound, C-279 (5.3 g, yield: 98%).

| | HOMO | LUMO | Triplet Energy | MP (° C.) | Tg (° C.) |
|---|---|---|---|---|---|
| C-279 | −4.848 | −1.177 | 2.419 | 234 | 106 |

### Device Example 1: Producing Red Light-Emitting OLED According to the Present Disclosure

An OLED according to the present disclosure was produced. First, a transparent electrode indium tin oxide (ITO) thin film (10Ω/sq) on a glass substrate for an OLED (GEOMATEC CO., LTD., Japan) was subjected to an ultrasonic washing with acetone and isopropyl alcohol, sequentially, and was then stored in isopropyl alcohol. The ITO substrate was mounted on a substrate holder of a vacuum vapor deposition apparatus. Compound HI-1 shown in Table 2, as the first hole injection compound, was introduced into a cell of the vacuum vapor deposition apparatus, and compound HT-1 shown in Table 2, as the first hole transport compound, was introduced into another cell. The two materials were evaporated at different rates, and the first hole injection compound was deposited in a doping amount of 3 wt % based to the total amount of the first hole injection compound and the first hole transport compound to form a first hole injection layer with a thickness of 10 nm. Subsequently, compound HT-1 was deposited on the first hole injection layer to form a first hole transport layer with a thickness of 90 nm. Next, compound C-86 shown in Table 2, as the second hole transport compound, was introduced into another cell of the vacuum vapor deposition apparatus and was evaporated by applying an electric current to the cell, thereby depositing a second hole transport layer with a thickness of 55 nm on the first hole transport layer. Next, compound H-31 shown in Table 2, as the third hole transport compound, was introduced into another cell of the vacuum vapor deposition apparatus and was evaporated by applying an electric current to the cell, thereby depositing a third hole transport layer with a thickness of 5 nm on the second hole transport layer. After forming the hole injection layer and the hole transport layers, a light-emitting layer was deposited thereon as follows: compound A-1 and compound B-1 were introduced into two cells of the vacuum vapor deposition apparatus as light-emitting layer hosts, and the compound A-1 and compound B-1 were evaporated at a rate of 1:1. After compound D-39 was introduced into another cell as a dopant, the dopant was then deposited in a doping amount of 2 wt % based on the total amount of the hosts and dopant to form a light-emitting layer with a thickness of 40 nm on the third hole transport layer. Subsequently, Compound B-2 was deposited to a thickness of 5 nm as an electron buffer material on the light-emitting layer. Then, compound ET-1 and compound EI-1 were doped at a weight ratio of 5:5 as an electron transport layer and deposited to a thickness of 30 nm. After depositing compound EI-1 as an electron injection layer with a thickness of 2 nm on the electron transport layer, an Al cathode was deposited with a thickness of 80 nm on the electron injection layer by using another vacuum vapor deposition apparatus to thereby produce an OLED.

### Device Examples 2 and 3: Producing Red Light-Emitting OLEDs According to the Present Disclosure

An OLED was produced in the same manner as in Device Example 1, except that compounds C-111 and C-1 shown in Table 2 were used in the second hole transport layer as second hole transport compounds instead of compound C-86.

### Device Example 4: Producing Red Light-Emitting OLED According to the Present Disclosure

An OLED was produced in the same manner as in Device Example 1, except that compound C-36 shown in Table 2 was used in the second hole transport layer as a second hole transport compound instead of compound C-86, and compound H-1 shown in Table 2 was used in the third hole transport layer as a third hole transport compound instead of compound H-31.

### Comparative Example 1: Producing Red Light-Emitting OLED Other than in Accordance with the Present Disclosure

An OLED was produced in the same manner as in Device Example 1, except that a single compound H-31 was deposited at 60 nm instead of using each compound in the second and third hole transport layers.

### Comparative Example 2: Producing Red Light-Emitting OLED Other than in Accordance with the Present Disclosure

An OLED was produced in the same manner as in Device Example 1, except that a single compound H-1 was deposited at 60 nm instead of using each compound in the second and third hole transport layers.

The driving voltage, current efficiency, and CIE color coordinates at a luminance of 1,000 nits of the OLED devices of Device Examples 1 to 4 and Comparative Examples 1 and 2 produced as described above, were measured, and the results thereof are shown in the following Table 1.

TABLE 1

| | Second | Third | | Current | |
|---|---|---|---|---|---|
| | Hole | Hole | Driving | Effi- | CIE Color |
| | Transport | Transport | Voltage | ciency | Coordinates |
| | Layer | Layer | (V) | (cd/A) | (x, y) |
| Device | C-86 | H-31 | 3.4 | 30.1 | (0.661, 0.339) |
| Example 1 | | | | | |
| Device | C-111 | H-31 | 3.8 | 32.2 | (0.661, 0.339) |
| Example 2 | | | | | |
| Device | C-1 | H-31 | 2.9 | 31.8 | (0.661, 0.339) |
| Example 3 | | | | | |
| Comparative | H-31 | | 5.6 | 28.6 | (0.661, 0.339) |
| Example 1 | | | | | |
| Device | C-36 | H-1 | 2.7 | 30.2 | (0.661, 0.338) |
| Example 4 | | | | | |
| Comparative | H-1 | | 4.6 | 29.1 | (0.661, 0.339) |
| Example 2 | | | | | |

From Table 1 above, it can be confirmed that the OLEDs according to the present disclosure exhibit a low driving voltage and high current efficiency properties compared to the OLEDs using the conventional compound in the second and third hole transport layers.

The compounds used in Device Examples 1 to 4 and Comparative Examples 1 and 2 are shown in Table 2 below.

TABLE 2

| | |
|---|---|
| Hole Injection Layer/ Hole Transport Layer | 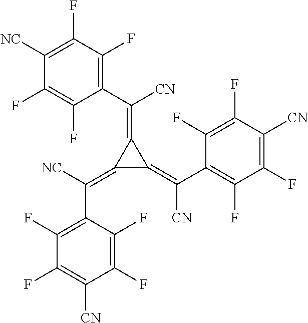 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-1 |
| | 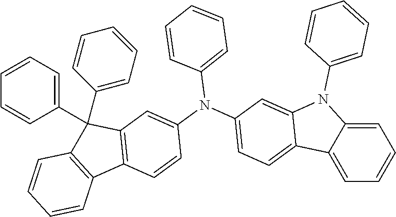 [SMILES: c1ccc(N(c2ccc3c(c2)C(c2ccccc2)(c2ccccc2)c2ccccc2-3)c2ccc3c4ccccc4n(-c4ccccc4)c3c2)cc1] |
| | HT-1 |
| | 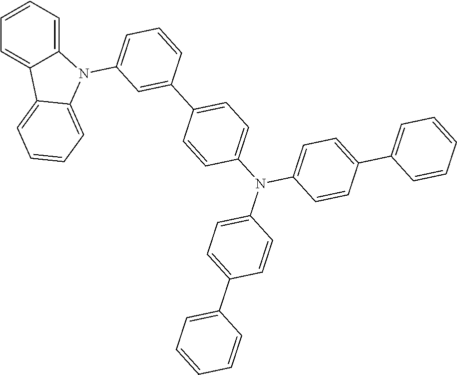 [SMILES: c1ccc(-c2ccc(N(c3ccc(-c4ccccc4)cc3)c3ccc(-c4cccc(-n5c6ccccc6c6ccccc65)c4)cc3)cc2)cc1] |
| | H-31 |
| | 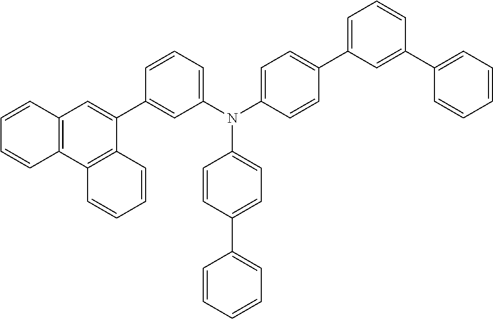 [SMILES: c1ccc(-c2ccc(N(c3ccc(-c4cccc(-c5ccccc5)c4)cc3)c3cccc(-c4cc5ccccc5c5ccccc45)c3)cc2)cc1] |
| | H-1 |
| | 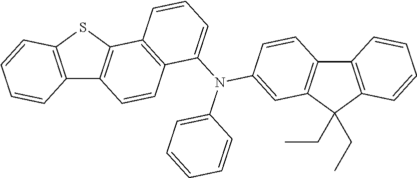 [SMILES: CCC1(CC)c2ccccc2-c2ccc(N(c3ccccc3)c3cccc4c3ccc3c5ccccc5sc43)cc21] |
| | C-86 |
| | 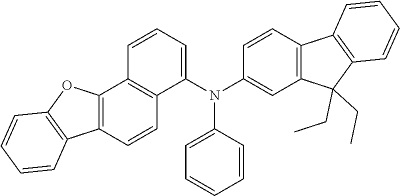 [SMILES: CCC1(CC)c2ccccc2-c2ccc(N(c3ccccc3)c3cccc4c3ccc3c5ccccc5oc43)cc21] |
| | C-111 |
| | 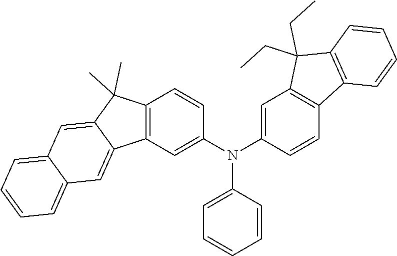 [SMILES: CCC1(CC)c2ccccc2-c2ccc(N(c3ccccc3)c3ccc4c(c3)-c3cc5ccccc5cc3C4(C)C)cc21] |
| | C-1 |
| | 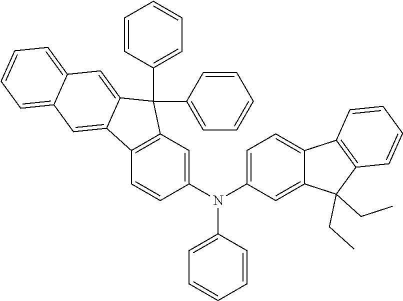 [SMILES: CCC1(CC)c2ccccc2-c2ccc(N(c3ccccc3)c3ccc4c(c3)C(c3ccccc3)(c3ccccc3)c3cc5ccccc5cc3-4)cc21] |
| | C-36 |
| Light-Emitting Layer | 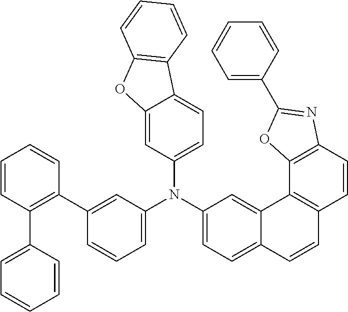 [SMILES: c1ccc(-c2nc3ccc4ccc5ccc(N(c6cccc(-c7ccccc7-c7ccccc7)c6)c6ccc7c(c6)oc6ccccc67)cc5c4c3o2)cc1] |
| | A-1 |
| | 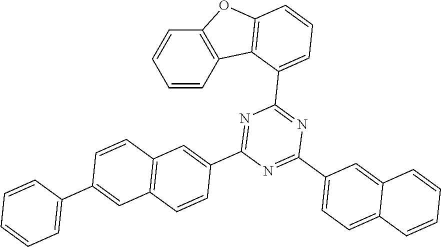 [SMILES: c1ccc(-c2ccc3cc(-c4nc(-c5ccc6ccccc6c5)nc(-c5cccc6oc7ccccc7c56)n4)ccc3c2)cc1] |
| | B-1 |
| | 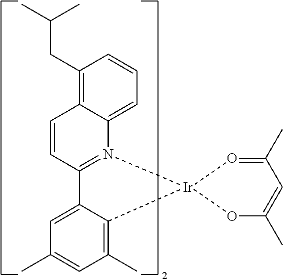 |
| | D-39 |
| Electron Buffer Layer/ Electron Transport Layer/ Electron Injection Layer | 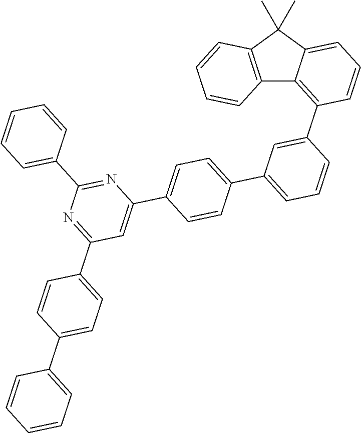 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21] |
| | B-2 |
| | 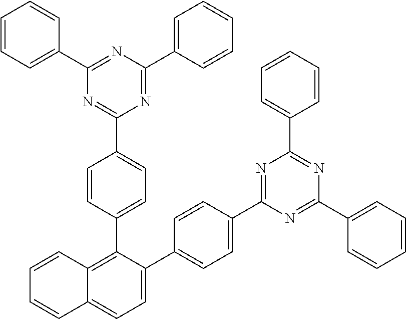 [SMILES: c1ccc(-c2nc(-c3ccccc3)nc(-c3ccc(-c4ccc5ccccc5c4-c4ccc(-c5nc(-c6ccccc6)nc(-c6ccccc6)n5)cc4)cc3)n2)cc1] |
| | ET-1 |
| | 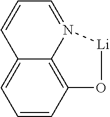 |
| | EI-1 |

### Device Example 5: Producing Red Light-Emitting OLED According to the Present Disclosure

An OLED according to the present disclosure was produced. First, a transparent electrode indium tin oxide (ITO) thin film (10Ω/sq) on a glass substrate for an OLED (GEOMATEC CO., LTD., Japan) was subjected to an ultrasonic washing with acetone and isopropyl alcohol, sequentially, and was then stored in isopropyl alcohol. The ITO substrate was mounted on a substrate holder of a vacuum vapor deposition apparatus. Compound HI-1 shown in Table 4, as the first hole injection compound, was introduced into a cell of the vacuum vapor deposition apparatus, and compound HT-2 shown in Table 4, as the first hole transport compound, was introduced into another cell. The two materials were evaporated at different rates, and the first hole injection compound was deposited in a doping amount of 5 wt % based to the total amount of the first hole injection compound and the first hole transport compound to form a first hole injection layer with a thickness of 10 nm. Subsequently, compound HT-2 was deposited on the first hole injection layer to form a first hole transport layer with a thickness of 90 nm. Next, compound C-279 shown in Table 4, as the second hole transport compound, was introduced into another cell of the vacuum vapor deposition apparatus and was evaporated by applying an electric current to the cell, thereby depositing a second hole transport layer with a thickness of 60 nm on the first hole transport layer. Next, compound H-56 shown in Table 4, as the third hole transport compound, was introduced into another cell of the vacuum vapor deposition apparatus and was evaporated by applying an electric current to the cell, thereby depositing a third hole transport layer with a thickness of 7.5 nm on the second hole transport layer. After forming the hole injection layer and the hole transport layers, a light-emitting layer was deposited thereon as follows: compound A-2 and compound B-1 were introduced into two cells of the vacuum vapor deposition apparatus as light-emitting layer hosts, and the compound A-2 and compound B-1 were evaporated at a rate of 1:1. After compound D-39 was introduced into another cell as a dopant, the dopant was then deposited in a doping amount of 2 wt % based on the total amount of the hosts and dopant to form a light-emitting layer with a thickness of 36 nm on the third hole transport layer. Subsequently, Compound B-3 was deposited to a thickness of 5 nm as an electron buffer material on the light-emitting layer. Then, compound ET-2 and compound EI-1 were doped at a weight ratio of 2:1 as an electron transport layer and deposited to a thickness of 25 nm. After depositing compound EI-1 as an electron injection layer with a thickness of 2 nm on the electron transport layer, an Al cathode was deposited with a thickness of 80 nm on the electron injection layer by using another vacuum vapor deposition apparatus to thereby produce an OLED.

### Device Example 6: Producing Red Light-Emitting OLED According to the Present Disclosure

An OLED was produced in the same manner as in Device Example 5, except that compound A-3 shown in Table 4 was used in the light-emitting layer as a light-emitting compound instead of compound A-2.

### Comparative Example 3: Producing Red Light-Emitting OLED Other than in Accordance with the Present Disclosure

An OLED was produced in the same manner as in Device Example 5, except that a single compound H-56 was deposited at 67.5 nm instead of using each compound in the second and third hole transport layers.

### Comparative Example 4: Producing Red Light-Emitting OLED Other than in Accordance with the Present Disclosure

An OLED was produced in the same manner as in Device Example 5, except that a single compound H-56 was deposited at 67.5 nm instead of using each compound in the second and third hole transport layers, and compound A-3 shown in Table 4 was used in the light-emitting layer as a light-emitting compound instead of compound A-2.

The driving voltage, current efficiency, and CIE color coordinates at a luminance of 1,000 nits of the OLED devices of Device Examples 5 and 6 and Comparative Examples 3 and 4 produced as described above were measured, and the results thereof are shown in the following Table 3.

TABLE 3

| | Second | Third | | | | |
|---|---|---|---|---|---|---|
| | Hole | Hole | Light- | Driving | Current | CIE Color |
| | Transport | Transport | Emitting | Voltage | Efficiency | Coordinates |
| | Layer | Layer | Host | (V) | (cd/A) | (x, y) |
| Device | C-279 | H-56 | A-2:B-1 | 2.9 | 29.2 | (0.661, 0.338) |
| Example 5 | | | | | | |
| Comparative | H-56 | | A-2:B-1 | 3.9 | 28.4 | (0.662, 0.338) |
| Example 3 | | | | | | |
| Device | C-279 | H-56 | A-3:B-1 | 2.9 | 28.6 | (0.661, 0.338) |
| Example 6 | | | | | | |
| Comparative | H-56 | | A-3:B-1 | 3.9 | 27.6 | (0.662, 0.338) |
| Example 4 | | | | | | |

From Table 3 above, it can be confirmed that the OLEDs according to the present disclosure exhibit a low driving voltage and high current efficiency properties compared to the OLEDs using the conventional compound in the second and third hole transport layers.

The compounds used in Device Examples 5 and 6 and Comparative Examples 3 and 4 are shown in Table 4 below.

TABLE 4

| | |
|---|---|
| Hole Injection Layer/ Hole Transport Layer | 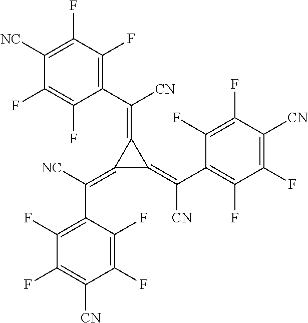 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-1 |
| | 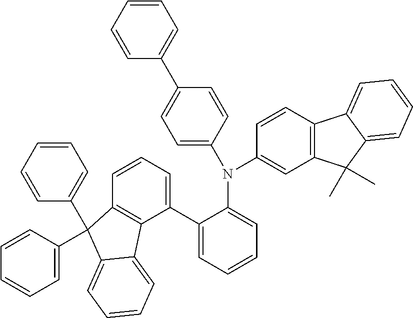 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21] |
| | HT-2 |
| | 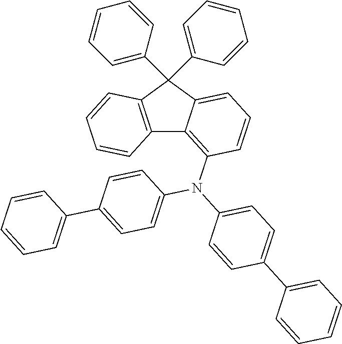 [SMILES: c1ccc(-c2ccc(N(c3ccc(-c4ccccc4)cc3)c3cccc4c3-c3ccccc3C4(c3ccccc3)c3ccccc3)cc2)cc1] |
| | H-56 |
| | 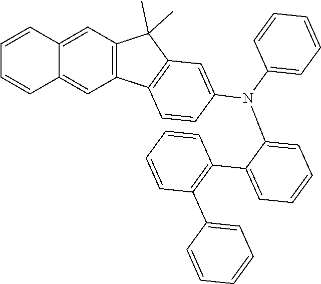 [SMILES: CC1(C)c2cc(N(c3ccccc3)c3ccccc3-c3ccccc3-c3ccccc3)ccc2-c2cc3ccccc3cc21] |
| | C-279 |
| Light-Emitting Layer | 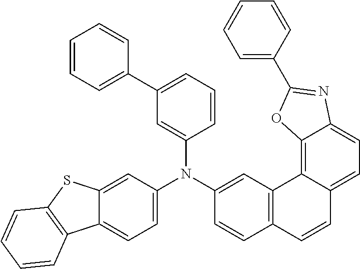 [SMILES: c1ccc(-c2cccc(N(c3ccc4c(c3)sc3ccccc34)c3ccc4ccc5ccc6nc(-c7ccccc7)oc6c5c4c3)c2)cc1] |
| | A-2 |
| | 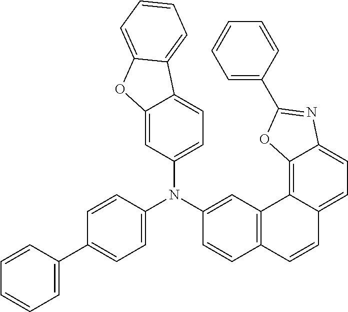 [SMILES: c1ccc(-c2ccc(N(c3ccc4c(c3)oc3ccccc34)c3ccc4ccc5ccc6nc(-c7ccccc7)oc6c5c4c3)cc2)cc1] |
| | A-3 |
| | 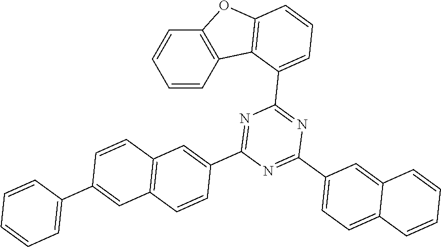 [SMILES: c1ccc(-c2ccc3cc(-c4nc(-c5ccc6ccccc6c5)nc(-c5cccc6oc7ccccc7c56)n4)ccc3c2)cc1] |
| | B-1 |
| | 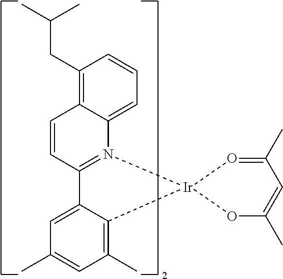 |
| | D-39 |
| Electron Buffer Layer/ Electron Transport Layer/ Electron Injection Layer | 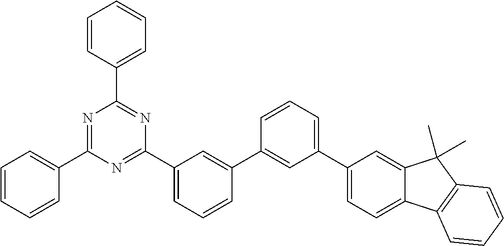 [SMILES: CC1(C)c2ccccc2-c2ccc(-c3cccc(-c4cccc(-c5nc(-c6ccccc6)nc(-c6ccccc6)n5)c4)c3)cc21] |
| | B-3 |
| Layer | 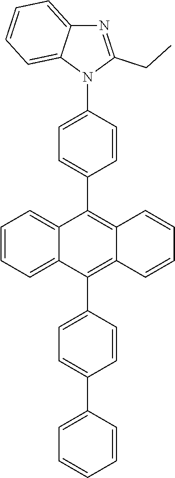 [SMILES: CCc1nc2ccccc2n1-c1ccc(-c2c3ccccc3c(-c3ccc(-c4ccccc4)cc3)c3ccccc23)cc1] |
| | ET-2 |
| | 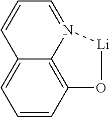 |
| | EI-1 |

### Device Example 7: Producing Red Light-Emitting OLED According to the Present Disclosure

An OLED according to the present disclosure was produced. First, a transparent electrode indium tin oxide (ITO) thin film (10Ω/sq) on a glass substrate for an OLED (GEOMATEC CO., LTD., Japan) was subjected to an ultrasonic washing with acetone and isopropyl alcohol, sequentially, and was then stored in isopropyl alcohol. The ITO substrate was mounted on a substrate holder of a vacuum vapor deposition apparatus. Compound HI-1 shown in Table 6, as the first hole injection compound, was introduced into a cell of the vacuum vapor deposition apparatus, and compound HT-1 shown in Table 6, as the first hole transport compound, was introduced into another cell. The two materials were evaporated at different rates, and the first hole injection compound was deposited in a doping amount of 3 wt % based to the total amount of the first hole injection compound and the first hole transport compound to form a first hole injection layer with a thickness of 10 nm. Subsequently, compound HT-1 was deposited on the first hole injection layer to form a first hole transport layer with a thickness of 90 nm. Next, compound C-1 shown in Table 6, as the second hole transport compound, was introduced into another cell of the vacuum vapor deposition apparatus and was evaporated by applying an electric current to the cell, thereby depositing a second hole transport layer with a thickness of 55 nm on the first hole transport layer. Next, compound H-13 shown in Table 6, as the third hole transport compound, was introduced into another cell of the vacuum vapor deposition apparatus and was evaporated by applying an electric current to the cell, thereby depositing a third hole transport layer with a thickness of 5 nm on the second hole transport layer. After forming the hole injection layer and the hole transport layers, a light-emitting layer was deposited thereon as follows: compound A-1 and compound B-1 were introduced into two cells of the vacuum vapor deposition apparatus as light-emitting layer hosts, and the compound A-1 and compound B-1 were evaporated at a rate of 4:6. After compound D-39 was introduced into another cell as a dopant, the dopant was then deposited in a doping amount of 2 wt % based on the total amount of the hosts and dopant to form a light-emitting layer with a thickness of 40 nm on the third hole transport layer. Subsequently, Compound B-2 was deposited to a thickness of 5 nm as an electron buffer material on the light-emitting layer. Then, compound ET-1 and compound EI-1 were doped at a weight ratio of 5:5 as an electron transport layer and deposited to a thickness of 30 nm. After depositing compound EI-1 as an electron injection layer with a thickness of 2 nm on the electron transport layer, an Al cathode was deposited with a thickness of 80 nm on the electron injection layer by using another vacuum vapor deposition apparatus to thereby produce an OLED.

### Device Examples 8 and 9: Producing Red Light-Emitting OLEDs According to the Present Disclosure

An OLED was produced in the same manner as in Device Example 7, except that each compound H-5 and H-114 shown in Table 5 was used in the third hole transport layer as a third hole transport compound instead of compound H-13.

The driving voltage, current efficiency, and CIE color coordinates at a luminance of 1,000 nits of the OLED devices of Device Examples 7 to 9 produced as described above were measured, and the results thereof are shown in the following Table 5.

TABLE 5

| | Second | Third | | | |
|---|---|---|---|---|---|
| | Hole | Hole | Driving | Current | CIE Color |
| | Transport | Transport | Voltage | Efficiency | Coordinates |
| | Layer | Layer | (V) | (cd/A) | (x, y) |
| Device | C-1 | H-13 | 2.8 | 33.4 | (0.657, 0.342) |
| Example 7 | | | | | |
| Device | C-1 | H-5 | 2.8 | 32.6 | (0.659, 0.340) |
| Example 8 | | | | | |
| Device | C-1 | H-114 | 2.8 | 33.2 | (0.658, 0.341) |
| Example 9 | | | | | |

From Table 5 above, it can be confirmed that the OLEDs according to the present disclosure exhibit a low driving voltage and high current efficiency properties.

The compounds used in Device Examples 7 to 9 are shown in Table 6 below.

TABLE 6

| | |
|---|---|
| Hole Injection Layer/ Hole Transport Layer | 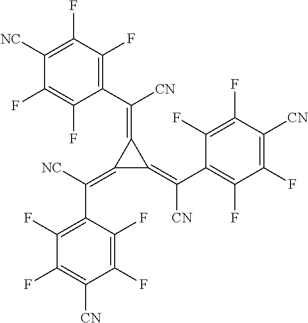 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-1 |
| | 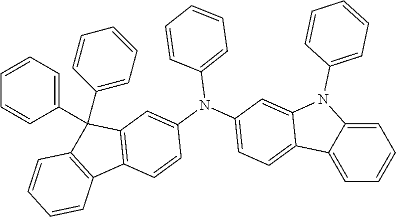 [SMILES: c1ccc(N(c2ccc3c(c2)C(c2ccccc2)(c2ccccc2)c2ccccc2-3)c2ccc3c4ccccc4n(-c4ccccc4)c3c2)cc1] |
| | HT-1 |
| | 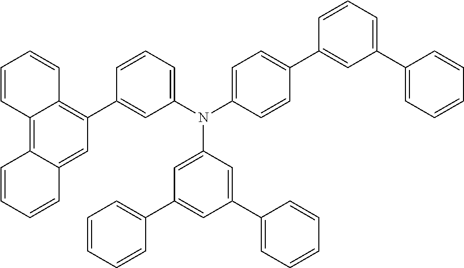 [SMILES: c1ccc(-c2cccc(-c3ccc(N(c4cc(-c5ccccc5)cc(-c5ccccc5)c4)c4cccc(-c5cc6ccccc6c6ccccc56)c4)cc3)c2)cc1] |
| | H-13 |
| | 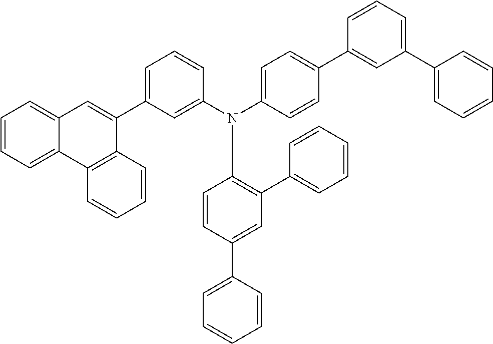 [SMILES: c1ccc(-c2cccc(-c3ccc(N(c4cccc(-c5cc6ccccc6c6ccccc56)c4)c4ccc(-c5ccccc5)cc4-c4ccccc4)cc3)c2)cc1] |
| | H-5 |
| | 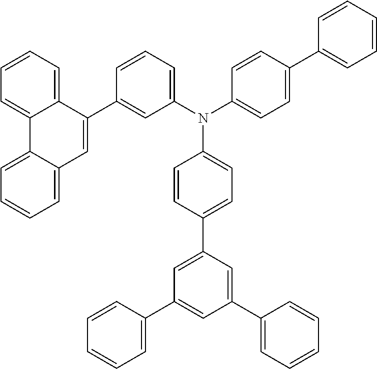 [SMILES: c1ccc(-c2ccc(N(c3ccc(-c4cc(-c5ccccc5)cc(-c5ccccc5)c4)cc3)c3cccc(-c4cc5ccccc5c5ccccc45)c3)cc2)cc1] |
| | H-114 |
| | 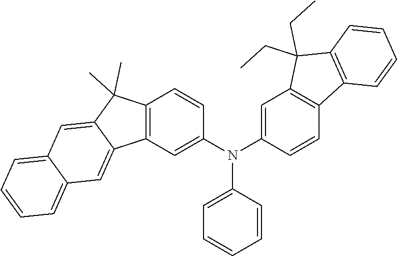 [SMILES: CCC1(CC)c2ccccc2-c2ccc(N(c3ccccc3)c3ccc4c(c3)-c3cc5ccccc5cc3C4(C)C)cc21] |
| | C-1 |
| Light-Emitting Layer | 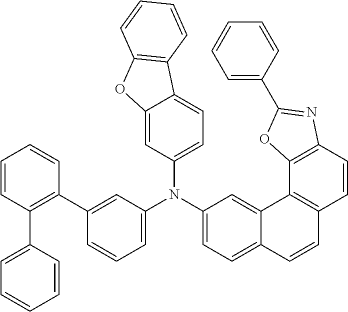 [SMILES: c1ccc(-c2nc3ccc4ccc5ccc(N(c6cccc(-c7ccccc7-c7ccccc7)c6)c6ccc7c(c6)oc6ccccc67)cc5c4c3o2)cc1] |
| | A-1 |
| | 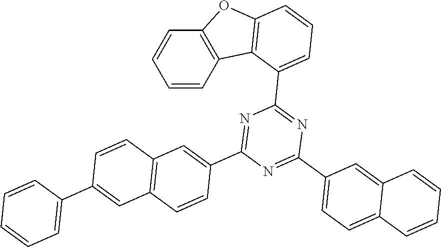 [SMILES: c1ccc(-c2ccc3cc(-c4nc(-c5ccc6ccccc6c5)nc(-c5cccc6oc7ccccc7c56)n4)ccc3c2)cc1] |
| | B-1 |
| | 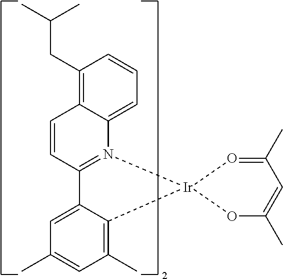 |
| | D-39 |
| Electron Buffer Layer/ Electron Transport Layer/ Electron Injection Layer | 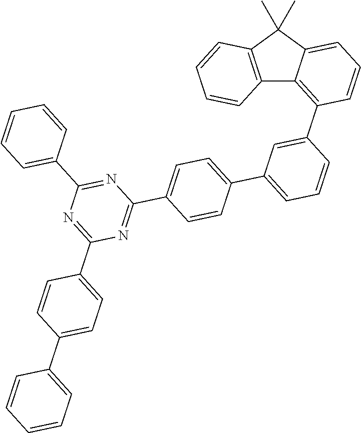 [SMILES: CC1(C)c2ccccc2-c2c(-c3cccc(-c4ccc(-c5nc(-c6ccccc6)nc(-c6ccc(-c7ccccc7)cc6)n5)cc4)c3)cccc21] |
| | B-2 |
| | 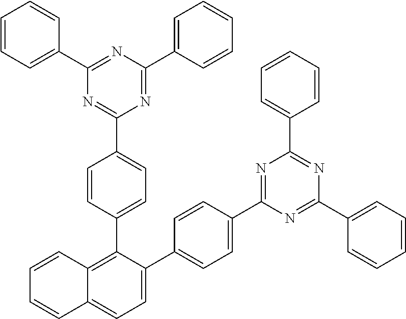 [SMILES: c1ccc(-c2nc(-c3ccccc3)nc(-c3ccc(-c4ccc5ccccc5c4-c4ccc(-c5nc(-c6ccccc6)nc(-c6ccccc6)n5)cc4)cc3)n2)cc1] |
| | ET-1 |
| | 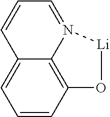 |
| | EI-1 |

### Device Example 10: Producing Red Light-Emitting OLED According to the Present Disclosure

An OLED according to the present disclosure was produced. First, a transparent electrode indium tin oxide (ITO) thin film (10Ω/sq) on a glass substrate for an OLED (GEOMATEC CO., LTD., Japan) was subjected to an ultrasonic washing with acetone and isopropyl alcohol, sequentially, and was then stored in isopropyl alcohol. The ITO substrate was mounted on a substrate holder of a vacuum vapor deposition apparatus. Compound HI-1 shown in Table 8, as the first hole injection compound, was introduced into a cell of the vacuum vapor deposition apparatus, and compound HT-1 shown in Table 8, as the first hole transport compound, was introduced into another cell. The two materials were evaporated at different rates, and the first hole injection compound was deposited in a doping amount of 3 wt % based to the total amount of the first hole injection compound and the first hole transport compound to form a first hole injection layer with a thickness of 10 nm. Subsequently, compound HT-1 was deposited on the first hole injection layer to form a first hole transport layer with a thickness of 90 nm. Next, compound C-351 shown in Table 8, as the second hole transport compound, was introduced into another cell of the vacuum vapor deposition apparatus and was evaporated by applying an electric current to the cell, thereby depositing a second hole transport layer with a thickness of 55 nm on the first hole transport layer. Next, compound H-5 shown in Table 8, as the third hole transport compound, was introduced into another cell of the vacuum vapor deposition apparatus and was evaporated by applying an electric current to the cell, thereby depositing a third hole transport layer with a thickness of 5 nm on the second hole transport layer. After forming the hole injection layer and the hole transport layers, a light-emitting layer was deposited thereon as follows: compound A-1 and compound B-1 were introduced into two cells of the vacuum vapor deposition apparatus as light-emitting layer hosts, and the compound A-1 and compound B-1 were evaporated at a rate of 1:1. After compound D-39 was introduced into another cell as a dopant, the dopant was then deposited in a doping amount of 2 wt % based on the total amount of the hosts and dopant to form a light-emitting layer with a thickness of 40 nm on the third hole transport layer. Subsequently, Compound B-2 was deposited to a thickness of 5 nm as an electron buffer material on the light-emitting layer. Then, compound ET-1 and compound EI-1 were doped at a weight ratio of 5:5 as an electron transport layer and deposited to a thickness of 30 nm. After depositing compound EI-1 as an electron injection layer with a thickness of 2 nm on the electron transport layer, an Al cathode was deposited with a thickness of 80 nm on the electron injection layer by using another vacuum vapor deposition apparatus to thereby produce an OLED.

### Device Examples 11 and 12: Producing Red Light-Emitting OLEDs According to the Present Disclosure

An OLED was produced in the same manner as in Device Example 10, except that each compound C-164 and C-163 shown in Table 7 was used in the second hole transport layer as a second hole transport compound instead of compound C-351.

The driving voltage, current efficiency, and CIE color coordinates at a luminance of 1,000 nits of the OLED devices of Device Examples 10 to 12 produced as described above were measured, and the results thereof are shown in the following Table 7.

TABLE 7

| | Second | Third | | Current | |
|---|---|---|---|---|---|
| | Hole | Hole | Driving | Effi- | CIE Color |
| | Transport | Transport | Voltage | ciency | Coordinates |
| | Layer | Layer | (V) | (cd/A) | (x, y) |
| Device | C-351 | H-5 | 2.9 | 32.5 | (0.660, 0.339) |
| Example 10 | | | | | |
| Device | C-164 | H-5 | 2.9 | 31.3 | (0.660, 0.339) |
| Example 11 | | | | | |
| Device | C-163 | H-5 | 2.7 | 31.7 | (0.660, 0.339) |
| Example 12 | | | | | |

From Table 7 above, it can be confirmed that the OLEDs according to the present disclosure exhibit a low driving voltage and high current efficiency properties.

The compounds used in Device Examples 10 to 12 are shown in Table 8 below.

TABLE 8

| | |
|---|---|
| Hole Injection Layer/ Hole Transport Layer | 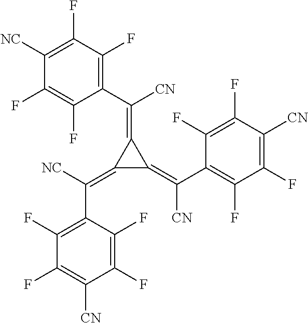 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-1 |
| | 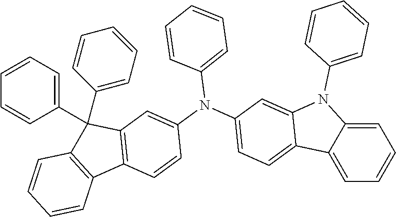 [SMILES: c1ccc(N(c2ccc3c(c2)C(c2ccccc2)(c2ccccc2)c2ccccc2-3)c2ccc3c4ccccc4n(-c4ccccc4)c3c2)cc1] |
| | HT-1 |
| | 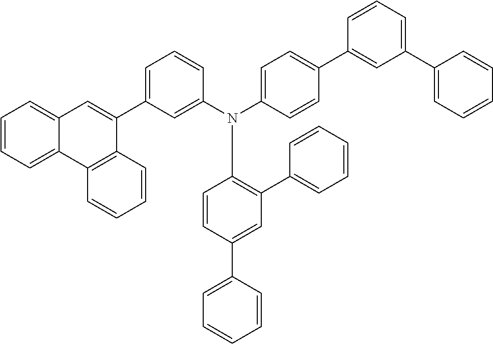 [SMILES: c1ccc(-c2cccc(-c3ccc(N(c4cccc(-c5cc6ccccc6c6ccccc56)c4)c4ccc(-c5ccccc5)cc4-c4ccccc4)cc3)c2)cc1] |
| | H-5 |
| | 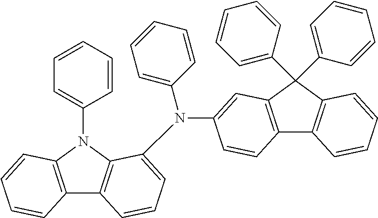 [SMILES: c1ccc(N(c2ccc3c(c2)C(c2ccccc2)(c2ccccc2)c2ccccc2-3)c2cccc3c4ccccc4n(-c4ccccc4)c23)cc1] |
| | C-351 |
| | 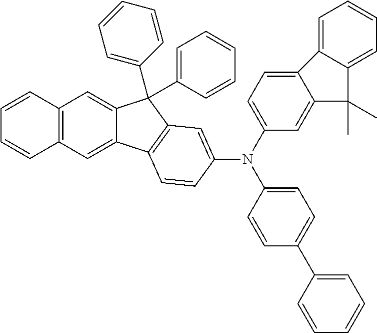 [SMILES: CC1(C)c2ccccc2-c2ccc(N(c3ccc(-c4ccccc4)cc3)c3ccc4c(c3)C(c3ccccc3)(c3ccccc3)c3cc5ccccc5cc3-4)cc21] |
| | C-164 |
| | 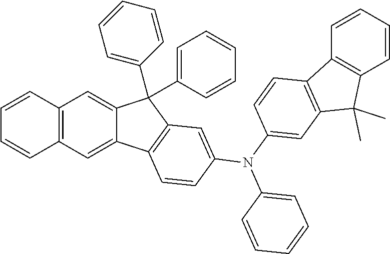 [SMILES: CC1(C)c2ccccc2-c2ccc(N(c3ccccc3)c3ccc4c(c3)C(c3ccccc3)(c3ccccc3)c3cc5ccccc5cc3-4)cc21] |
| | C-163 |
| Light-Emitting Layer | 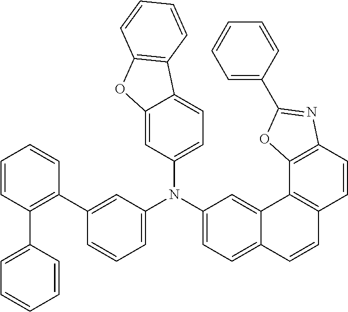 [SMILES: c1ccc(-c2nc3ccc4ccc5ccc(N(c6cccc(-c7ccccc7-c7ccccc7)c6)c6ccc7c(c6)oc6ccccc67)cc5c4c3o2)cc1] |
| | A-1 |
| | 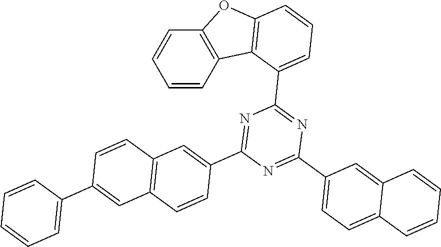 [SMILES: c1ccc(-c2ccc3cc(-c4nc(-c5ccc6ccccc6c5)nc(-c5cccc6oc7ccccc7c56)n4)ccc3c2)cc1] |
| | B-1 |
| | 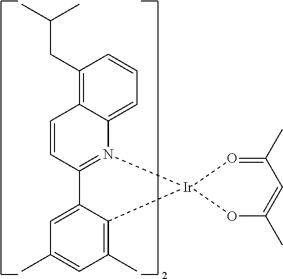 |
| | D-39 |
| Electron Buffer Layer/ Electron Transport Layer/ Electron Injection Layer | 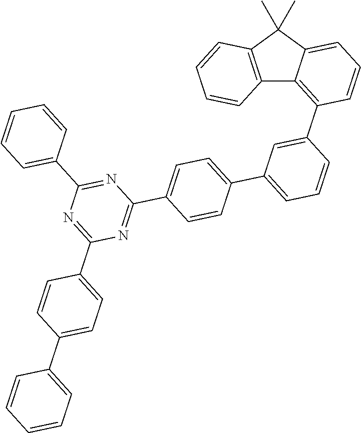 [SMILES: CC1(C)c2ccccc2-c2c(-c3cccc(-c4ccc(-c5nc(-c6ccccc6)nc(-c6ccc(-c7ccccc7)cc6)n5)cc4)c3)cccc21] |
| | B-2 |
| | 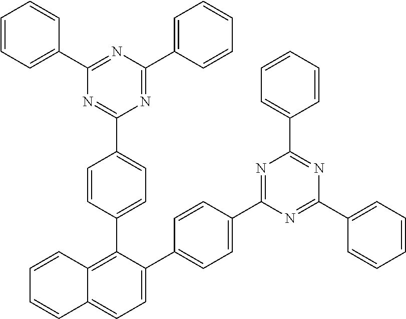 [SMILES: c1ccc(-c2nc(-c3ccccc3)nc(-c3ccc(-c4ccc5ccccc5c4-c4ccc(-c5nc(-c6ccccc6)nc(-c6ccccc6)n5)cc4)cc3)n2)cc1] |
| | ET-1 |
| | 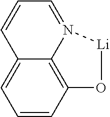 |
| | EI-1 |

### Device Example 13: Producing Red Light-Emitting OLED According to the Present Disclosure

An OLED according to the present disclosure was produced. First, a transparent electrode indium tin oxide (ITO) thin film (10Ω/sq) on a glass substrate for an OLED (GEOMATEC CO., LTD., Japan) was subjected to an ultrasonic washing with acetone and isopropyl alcohol, sequentially, and was then stored in isopropyl alcohol. The ITO substrate was mounted on a substrate holder of a vacuum vapor deposition apparatus. Compound HI-1 shown in Table 10, as the first hole injection compound, was introduced into a cell of the vacuum vapor deposition apparatus, and compound HT-2 shown in Table 10, as the first hole transport compound, was introduced into another cell. The two materials were evaporated at different rates, and the first hole injection compound was deposited in a doping amount of 5 wt % based to the total amount of the first hole injection compound and the first hole transport compound to form a first hole injection layer with a thickness of 10 nm. Subsequently, compound HT-2 was deposited on the first hole injection layer to form a first hole transport layer with a thickness of 90 nm. Next, compound C-272 shown in Table 10, as the second hole transport compound, was introduced into another cell of the vacuum vapor deposition apparatus and was evaporated by applying an electric current to the cell, thereby depositing a second hole transport layer with a thickness of 60 nm on the first hole transport layer. Next, compound H-56 shown in Table 10, as the third hole transport compound, was introduced into another cell of the vacuum vapor deposition apparatus and was evaporated by applying an electric current to the cell, thereby depositing a third hole transport layer with a thickness of 7.5 nm on the second hole transport layer. After forming the hole injection layer and the hole transport layers, a light-emitting layer was deposited thereon as follows: compound A-2 and compound B-1 were introduced into two cells of the vacuum vapor deposition apparatus as light-emitting layer hosts, and the compound A-2 and compound B-1 were evaporated at a rate of 1:1. After compound D-39 was introduced into another cell as a dopant, the dopant was then deposited in a doping amount of 2 wt % based on the total amount of the hosts and dopant to form a light-emitting layer with a thickness of 36 nm on the third hole transport layer. Subsequently, Compound B-3 was deposited to a thickness of 5 nm as an electron buffer material on the light-emitting layer. Then, compound ET-2 and compound EI-1 were doped at a weight ratio of 2:1 as an electron transport layer and deposited to a thickness of 25 nm. After depositing compound EI-1 as an electron injection layer with a thickness of 2 nm on the electron transport layer, an Al cathode was deposited with a thickness of 80 nm on the electron injection layer by using another vacuum vapor deposition apparatus to thereby produce an OLED.

### Device Examples 14 to 23: Producing Red Light-Emitting OLEDs According to the Present Disclosure

An OLED was produced in the same manner as in Device Example 13, except that each compound shown in Table 9 was used in the second hole transport layer as a second hole transport compound instead of compound C-272.

The driving voltage, current efficiency, and CIE color coordinates at a luminance of 1,000 nits of the OLED devices of Device Examples 10 to 12 produced as described above were measured, and the results thereof are shown in the following Table 9.

TABLE 9

| | Second | Third | | | | |
|---|---|---|---|---|---|---|
| | Hole | Hole | Light- | Driving | Current | CIE Color |
| | Transport | Transport | Emitting | Voltage | Efficiency | Coordinates |
| | Layer | Layer | Host | (V) | (cd/A) | (x, y) |
| Device | C-272 | H-56 | A-2:B-1 | 3.0 | 29.5 | (0.662, 0.338) |
| Example 13 | | | | | | |
| Device | C-347 | H-56 | A-2:B-1 | 3.2 | 29.4 | (0.662, 0.337) |
| Example 14 | | | | | | |
| Device | C-11 | H-56 | A-2:B-1 | 2.9 | 30.0 | (0.662, 0.338) |
| Example 15 | | | | | | |
| Device | C-257 | H-56 | A-2:B-1 | 3.0 | 30.6 | (0.661, 0.338) |
| Example 16 | | | | | | |
| Device | C-252 | H-56 | A-2:B-1 | 2.9 | 29.6 | (0.662, 0.338) |
| Example 17 | | | | | | |
| Device | C-18 | H-56 | A-2:B-1 | 3.0 | 30.9 | (0.662, 0.338) |
| Example 18 | | | | | | |
| Device | C-381 | H-56 | A-2:B-1 | 2.9 | 30.6 | (0.662, 0.338) |
| Example 19 | | | | | | |
| Device | C-382 | H-56 | A-2:B-1 | 2.9 | 31.3 | (0.662, 0.338) |
| Example 20 | | | | | | |
| Device | C-383 | H-56 | A-2:B-1 | 2.8 | 29.5 | (0.662, 0.338) |
| Example 21 | | | | | | |
| Device | C-384 | H-56 | A-2:B-1 | 2.9 | 30.2 | (0.662, 0.338) |
| Example 22 | | | | | | |
| Device | C-385 | H-56 | A-2:B-1 | 2.9 | 30.8 | (0.661, 0.338) |
| Example 23 | | | | | | |

From Table 9 above, it can be confirmed that the OLEDs according to the present disclosure exhibit a low driving voltage and high current efficiency properties.

The compounds used in Device Examples 13 to 23 are shown in Table 10 below.

TABLE 10

| | |
|---|---|
| Hole Injection Layer/ Hole Transport Layer | 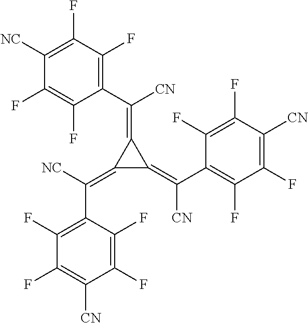 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-1 |
| | 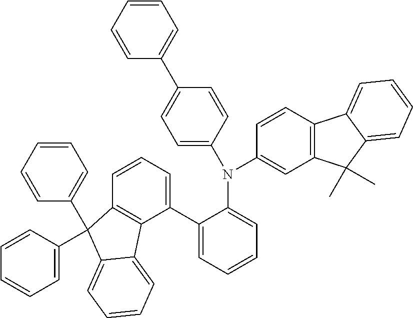 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21] |
| | HT-2 |
| | 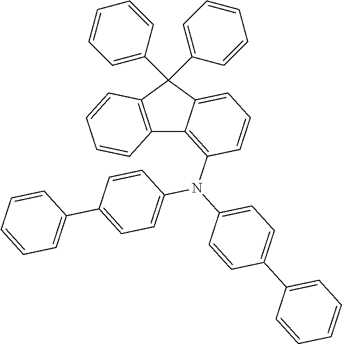 [SMILES: c1ccc(-c2ccc(N(c3ccc(-c4ccccc4)cc3)c3cccc4c3-c3ccccc3C4(c3ccccc3)c3ccccc3)cc2)cc1] |
| | H-56 |
| | 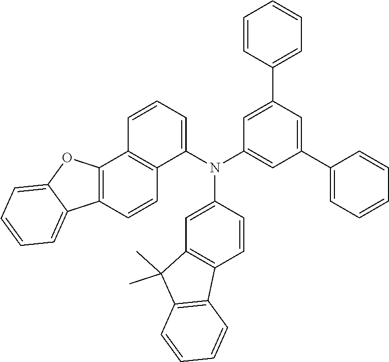 [SMILES: CC1(C)c2ccccc2-c2ccc(N(c3cc(-c4ccccc4)cc(-c4ccccc4)c3)c3cccc4c3ccc3c5ccccc5oc43)cc21] |
| | C-272 |
| | 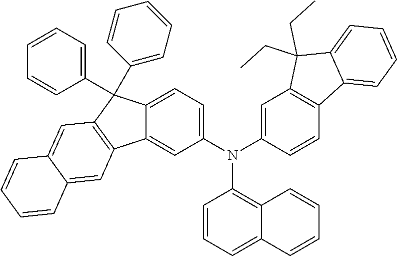 [SMILES: CCC1(CC)c2ccccc2-c2ccc(N(c3ccc4c(c3)-c3cc5ccccc5cc3C4(c3ccccc3)c3ccccc3)c3cccc4ccccc34)cc21] |
| | C-347 |
| | 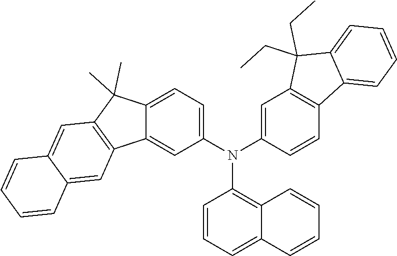 [SMILES: CCC1(CC)c2ccccc2-c2ccc(N(c3ccc4c(c3)-c3cc5ccccc5cc3C4(C)C)c3cccc4ccccc34)cc21] |
| | C-11 |
| | 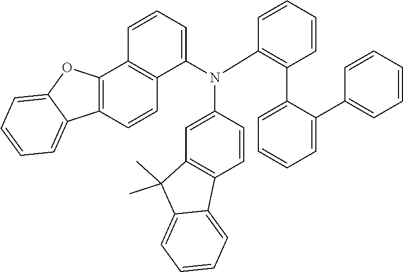 [SMILES: CC1(C)c2ccccc2-c2ccc(N(c3ccccc3-c3ccccc3-c3ccccc3)c3cccc4c3ccc3c5ccccc5oc43)cc21] |
| | C-257 |
| | 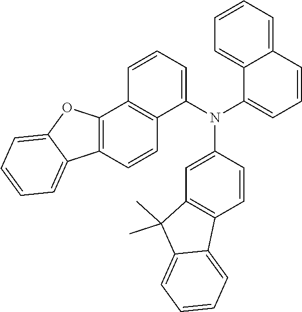 [SMILES: CC1(C)c2ccccc2-c2ccc(N(c3cccc4ccccc34)c3cccc4c3ccc3c5ccccc5oc43)cc21] |
| | C-252 |
| | 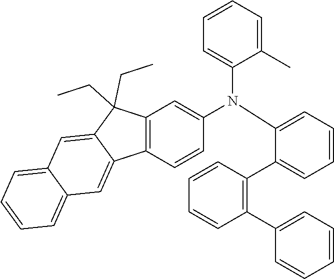 [SMILES: CCC1(CC)c2cc(N(c3ccccc3C)c3ccccc3-c3ccccc3-c3ccccc3)ccc2-c2cc3ccccc3cc21] |
| | C-18 |
| | 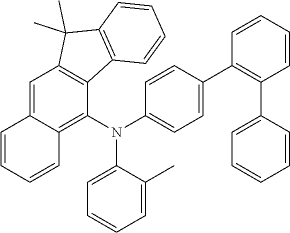 [SMILES: Cc1ccccc1N(c1ccc(-c2ccccc2-c2ccccc2)cc1)c1c2c(cc3ccccc13)C(C)(C)c1ccccc1-2] |
| | C-381 |
| | 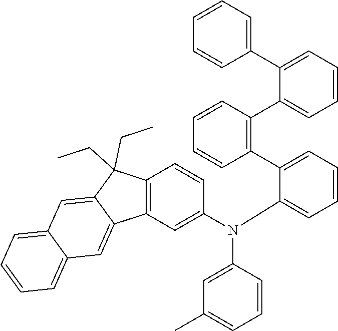 [SMILES: CCC1(CC)c2ccc(N(c3cccc(C)c3)c3ccccc3-c3ccccc3-c3ccccc3-c3ccccc3)cc2-c2cc3ccccc3cc21] |
| | C-382 |
| | 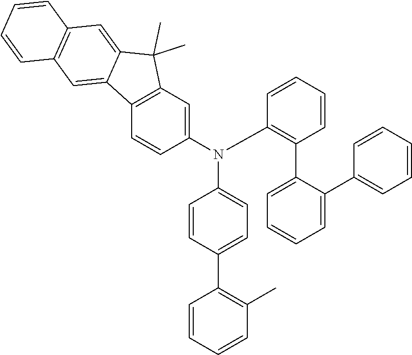 [SMILES: Cc1ccccc1-c1ccc(N(c2ccc3c(c2)C(C)(C)c2cc4ccccc4cc2-3)c2ccccc2-c2ccccc2-c2ccccc2)cc1] |
| | C-383 |
| | 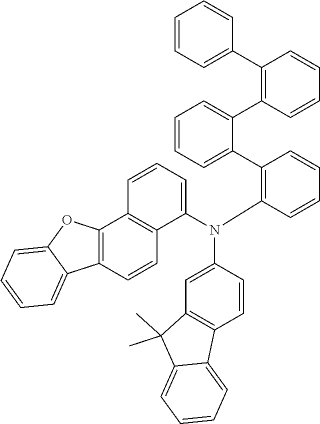 [SMILES: CC1(C)c2ccccc2-c2ccc(N(c3ccccc3-c3ccccc3-c3ccccc3-c3ccccc3)c3cccc4c3ccc3c5ccccc5oc43)cc21] |
| | C-384 |
| | 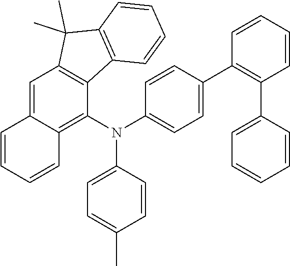 [SMILES: Cc1ccc(N(c2ccc(-c3ccccc3-c3ccccc3)cc2)c2c3c(cc4ccccc24)C(C)(C)c2ccccc2-3)cc1] |
| | C-385 |
| Light-Emitting Layer | 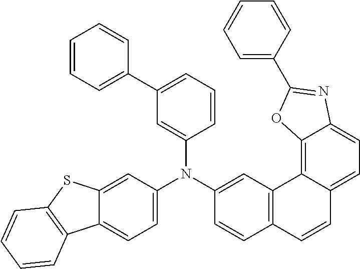 [SMILES: c1ccc(-c2cccc(N(c3ccc4c(c3)sc3ccccc34)c3ccc4ccc5ccc6nc(-c7ccccc7)oc6c5c4c3)c2)cc1] |
| | A-2 |
| | 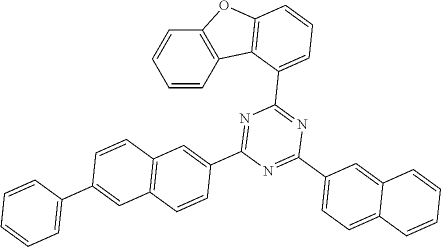 [SMILES: c1ccc(-c2ccc3cc(-c4nc(-c5ccc6ccccc6c5)nc(-c5cccc6oc7ccccc7c56)n4)ccc3c2)cc1] |
| | B-1 |
| | 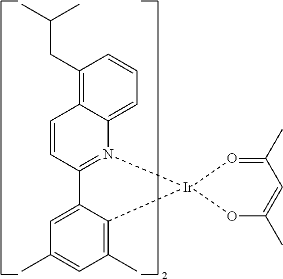 |
| | D-39 |
| Electron Buffer Layer/ Electron Transport Layer/ Electron Injection Layer | 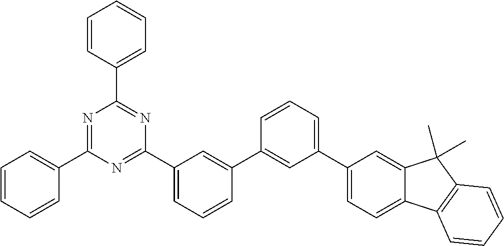 [SMILES: CC1(C)c2ccccc2-c2ccc(-c3cccc(-c4cccc(-c5nc(-c6ccccc6)nc(-c6ccccc6)n5)c4)c3)cc21] |
| | B-3 |
| | 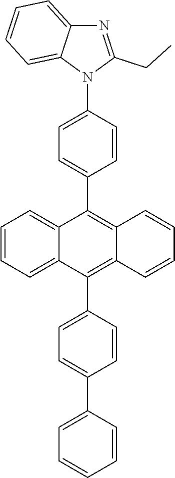 [SMILES: CCc1nc2ccccc2n1-c1ccc(-c2c3ccccc3c(-c3ccc(-c4ccccc4)cc3)c3ccccc23)cc1] |
| | ET-2 |
| | 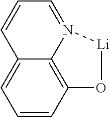 |
| | EI-1 |

1. An organic electroluminescent device comprising:
a first electrode; a second electrode facing the first electrode; a structure in which a hole injection layer, a first hole transport layer, a second hole transport layer, a third transport layer, and a light-emitting layer are sequentially stacked between the first electrode and the second electrode,
wherein the structures of the compounds comprised in the first hole transport layer, the second hole transport layer, and the third hole transport layer are each different;
wherein the second hole transport layer comprises a compound comprising a fluorene-amine, benzofluorene-amine, or naphthofluorene-amine structure.

2. The organic electroluminescent device according to claim 1, wherein the compound comprising the fluorene-amine, benzofluorene-amine, or naphthofluorene-amine structure represented by the following Formula 1: 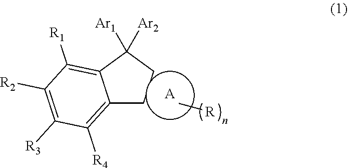
wherein in Formula 1,
ring A represents a substituted or unsubstituted benzene, a substituted or unsubstituted naphthalene, or a substituted or unsubstituted phenanthrene;
Ar₁ and Ar₂ each independently represent a substituted or unsubstituted (C1-C30)alkyl, a substituted or unsubstituted (C6-C30)aryl, or may be linked to each other to form a ring(s);
R and R₁ to R₄ each independently represent hydrogen, deuterium, halogen, cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, or a substituted or unsubstituted (C1-C30)alkoxy, or may be linked to an adjacent substituent(s) to form a ring(s); with a proviso that at least one of R and R₁ to R₄ is represented by -L₁-NAr₃Ar₄;
L₁ represents a single bond, a substituted or unsubstituted (C6-C30)arylene, or a substituted or unsubstituted (3- to 30-membered)heteroarylene;
Ar₃ and Ar₄ each independently represent a substituted or unsubstituted (C6-C30)aryl, or a substituted or unsubstituted (3- to 30-membered)heteroaryl; and
n represents an integer of 1 to 8, if n is plural, each of n may be the same as or different from each other.

3. The organic electroluminescent device according to claim 2, wherein the compound represented by the Formula 1 is represented by any one of the following Formulas 1-1 to 1-11: 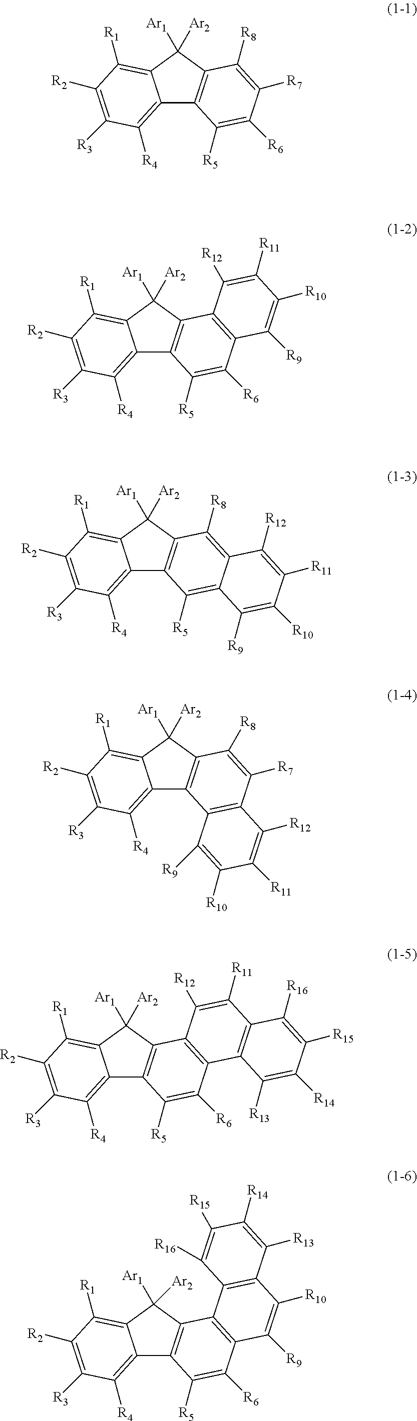 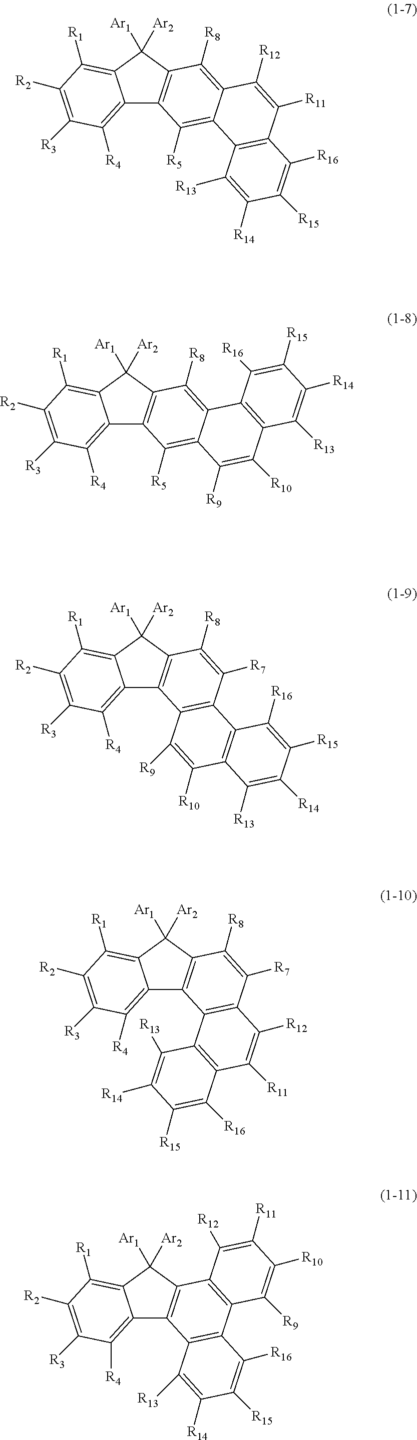
wherein in Formulas 1-1 to 1-11,
Ar₁ and Ar₂ each independently represent a substituted or unsubstituted (C1-C30)alkyl, a substituted or unsubstituted (C6-C30)aryl, or may be linked to each other to form a fused ring(s);
R₁ to R₁₆ each independently represents hydrogen, deuterium, a halogen, a cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, or a substituted or unsubstituted (C1-C30)alkoxy, or may be linked to an adjacent substituent(s) to form a ring(s); with a proviso that at least one of R₁ to R₁₆ is represented by -L₁-NAr₃Ar₄;
L₁ represents a single bond, a substituted or unsubstituted (C6-C30)arylene, or a substituted or unsubstituted (3- to 30-membered)heteroarylene; and
Ar₃ and Ar₄ each independently represent a substituted or unsubstituted (C6-C30)aryl, or a substituted or unsubstituted (3- to 30-membered)heteroaryl.

4. The organic electroluminescent device according to claim 2, wherein the compound represented by Formula 1 is at least one selected from the following compounds: 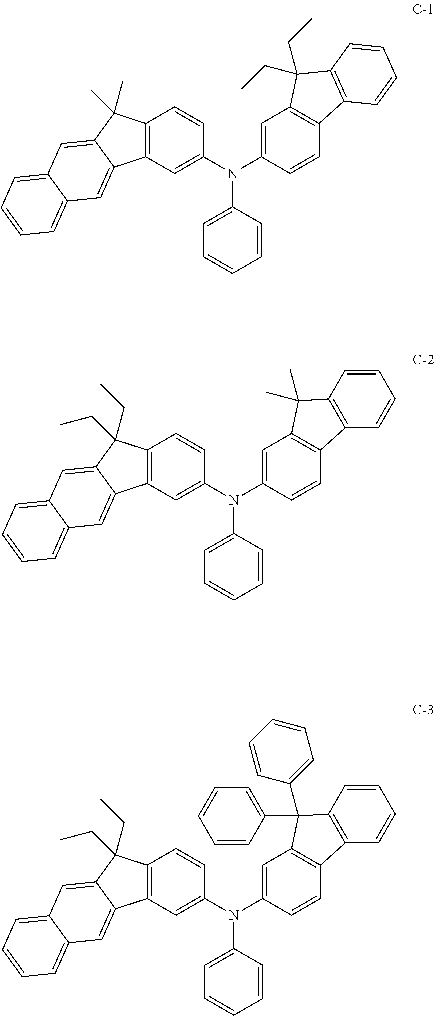 [SMILES: CCC1(CC)c2ccccc2-c2ccc(N(c3ccccc3)c3ccc4c(c3)-c3cc5ccccc5cc3C4(C)C)cc21 ; CCC1(CC)c2ccc(N(c3ccccc3)c3ccc4c(c3)C(C)(C)c3ccccc3-4)cc2-c2cc3ccccc3cc21 ; CCC1(CC)c2ccc(N(c3ccccc3)c3ccc4c(c3)C(c3ccccc3)(c3ccccc3)c3ccccc3-4)cc2-c2cc3ccccc3cc21] 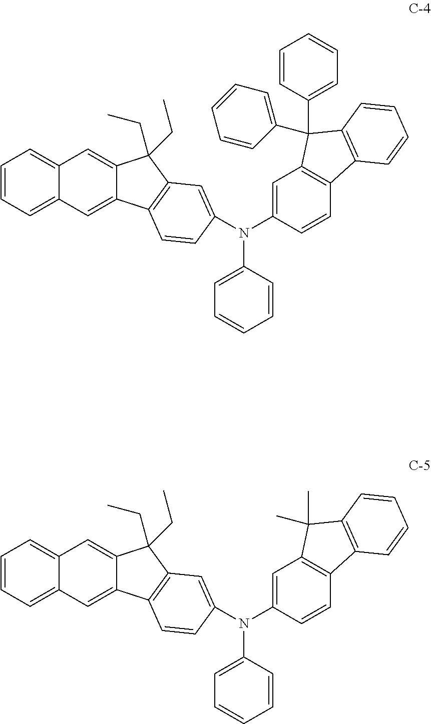 [SMILES: CCC1(CC)c2cc(N(c3ccccc3)c3ccc4c(c3)C(c3ccccc3)(c3ccccc3)c3ccccc3-4)ccc2-c2cc3ccccc3cc21 ; CCC1(CC)c2cc(N(c3ccccc3)c3ccc4c(c3)C(C)(C)c3ccccc3-4)ccc2-c2cc3ccccc3cc21] 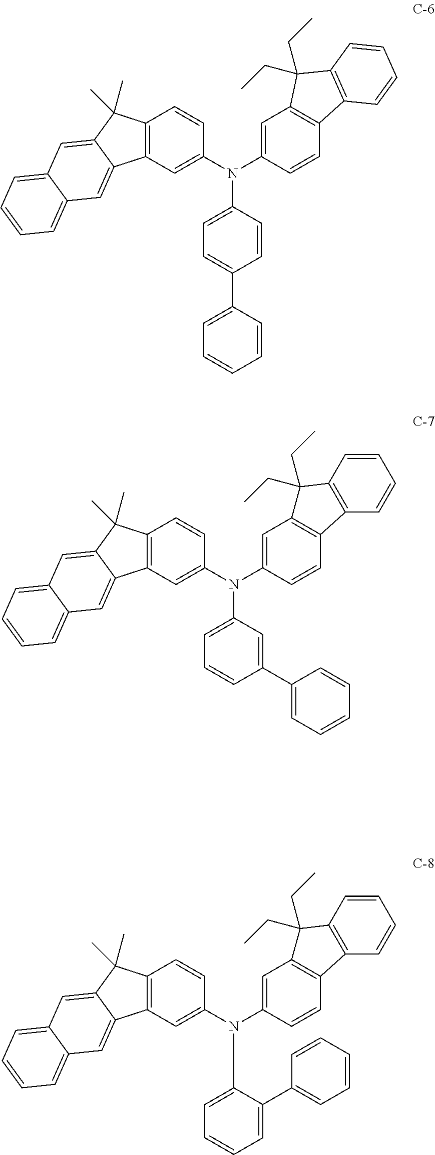 [SMILES: CCC1(CC)c2ccccc2-c2ccc(N(c3ccc(-c4ccccc4)cc3)c3ccc4c(c3)-c3cc5ccccc5cc3C4(C)C)cc21 ; CCC1(CC)c2ccccc2-c2ccc(N(c3cccc(-c4ccccc4)c3)c3ccc4c(c3)-c3cc5ccccc5cc3C4(C)C)cc21 ; CCC1(CC)c2ccccc2-c2ccc(N(c3ccc4c(c3)-c3cc5ccccc5cc3C4(C)C)c3ccccc3-c3ccccc3)cc21] 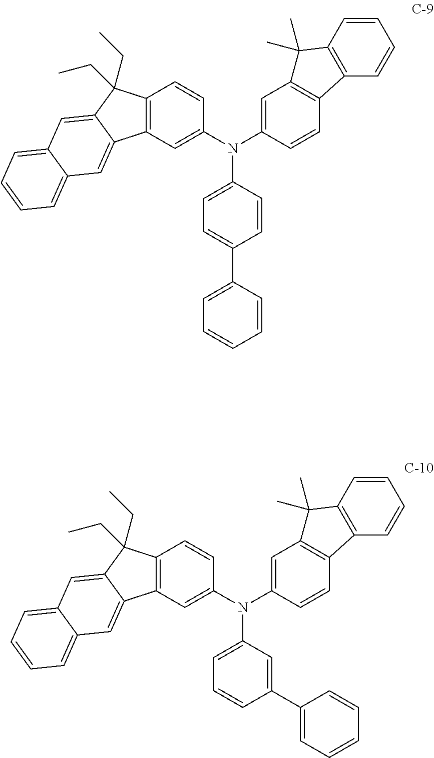 [SMILES: CCC1(CC)c2ccc(N(c3ccc(-c4ccccc4)cc3)c3ccc4c(c3)C(C)(C)c3ccccc3-4)cc2-c2cc3ccccc3cc21 ; CCC1(CC)c2ccc(N(c3cccc(-c4ccccc4)c3)c3ccc4c(c3)C(C)(C)c3ccccc3-4)cc2-c2cc3ccccc3cc21] 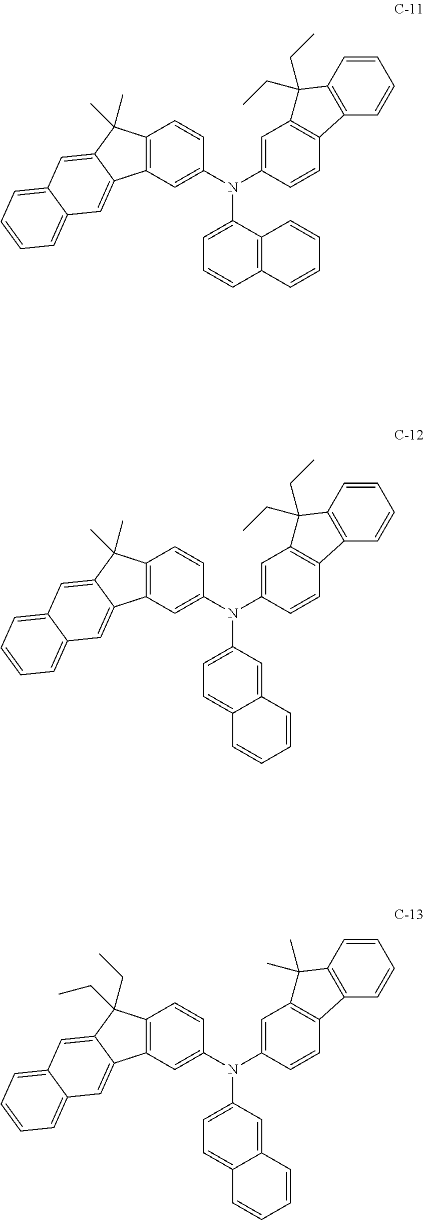 [SMILES: CCC1(CC)c2ccccc2-c2ccc(N(c3ccc4c(c3)-c3cc5ccccc5cc3C4(C)C)c3cccc4ccccc34)cc21 ; CCC1(CC)c2ccccc2-c2ccc(N(c3ccc4c(c3)-c3cc5ccccc5cc3C4(C)C)c3ccc4ccccc4c3)cc21 ; CCC1(CC)c2ccc(N(c3ccc4c(c3)C(C)(C)c3ccccc3-4)c3ccc4ccccc4c3)cc2-c2cc3ccccc3cc21] 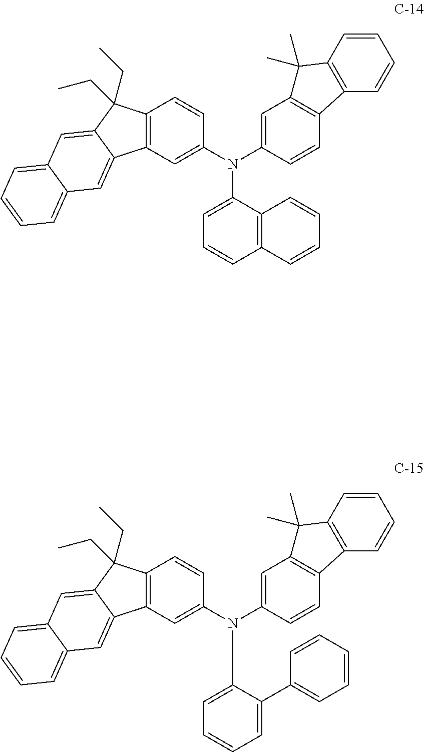 [SMILES: CCC1(CC)c2ccc(N(c3ccc4c(c3)C(C)(C)c3ccccc3-4)c3cccc4ccccc34)cc2-c2cc3ccccc3cc21 ; CCC1(CC)c2ccc(N(c3ccc4c(c3)C(C)(C)c3ccccc3-4)c3ccccc3-c3ccccc3)cc2-c2cc3ccccc3cc21] 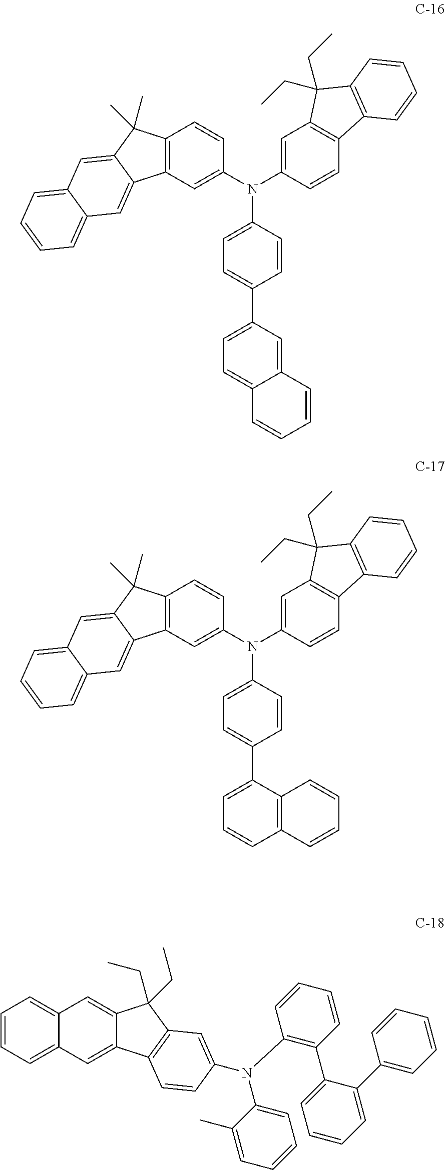 [SMILES: CCC1(CC)c2ccccc2-c2ccc(N(c3ccc(-c4ccc5ccccc5c4)cc3)c3ccc4c(c3)-c3cc5ccccc5cc3C4(C)C)cc21 ; CCC1(CC)c2ccccc2-c2ccc(N(c3ccc(-c4cccc5ccccc45)cc3)c3ccc4c(c3)-c3cc5ccccc5cc3C4(C)C)cc21 ; CCC1(CC)c2cc(N(c3ccccc3C)c3ccccc3-c3ccccc3-c3ccccc3)ccc2-c2cc3ccccc3cc21] 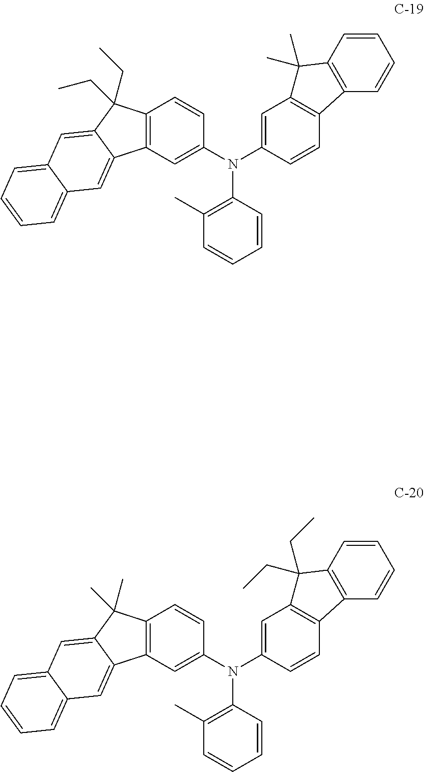 [SMILES: CCC1(CC)c2ccc(N(c3ccc4c(c3)C(C)(C)c3ccccc3-4)c3ccccc3C)cc2-c2cc3ccccc3cc21 ; CCC1(CC)c2ccccc2-c2ccc(N(c3ccc4c(c3)-c3cc5ccccc5cc3C4(C)C)c3ccccc3C)cc21] 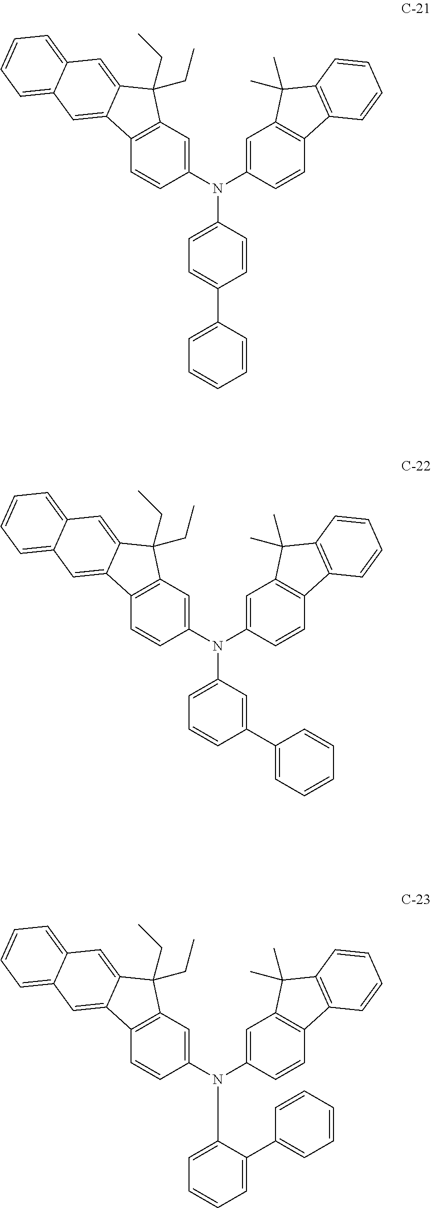 [SMILES: CCC1(CC)c2cc(N(c3ccc(-c4ccccc4)cc3)c3ccc4c(c3)C(C)(C)c3ccccc3-4)ccc2-c2cc3ccccc3cc21 ; CCC1(CC)c2cc(N(c3cccc(-c4ccccc4)c3)c3ccc4c(c3)C(C)(C)c3ccccc3-4)ccc2-c2cc3ccccc3cc21 ; CCC1(CC)c2cc(N(c3ccc4c(c3)C(C)(C)c3ccccc3-4)c3ccccc3-c3ccccc3)ccc2-c2cc3ccccc3cc21] 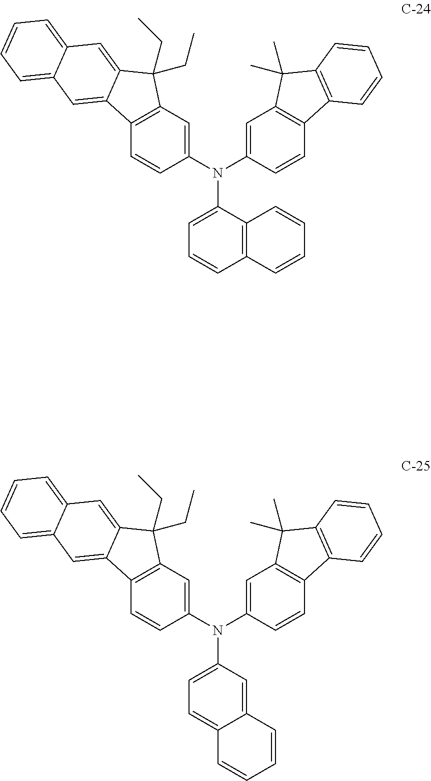 [SMILES: CCC1(CC)c2cc(N(c3ccc4c(c3)C(C)(C)c3ccccc3-4)c3cccc4ccccc34)ccc2-c2cc3ccccc3cc21 ; CCC1(CC)c2cc(N(c3ccc4c(c3)C(C)(C)c3ccccc3-4)c3ccc4ccccc4c3)ccc2-c2cc3ccccc3cc21] 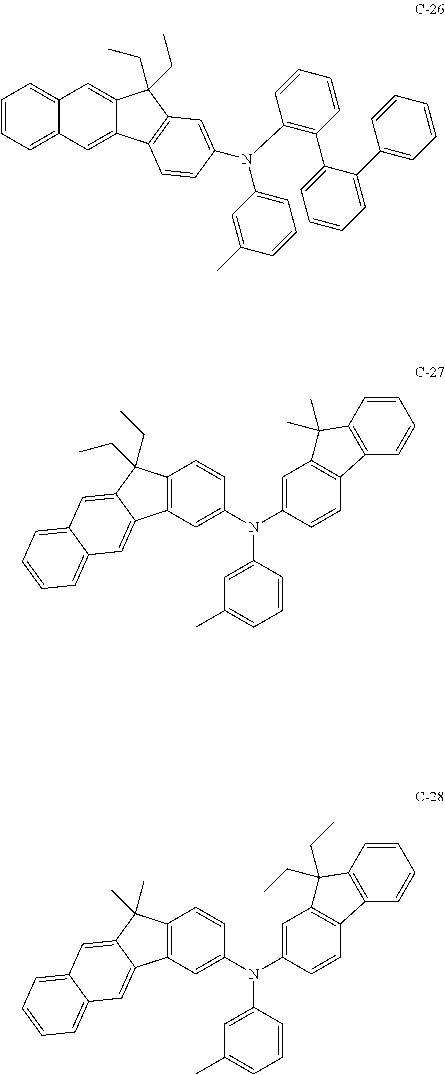 [SMILES: CCC1(CC)c2cc(N(c3cccc(C)c3)c3ccccc3-c3ccccc3-c3ccccc3)ccc2-c2cc3ccccc3cc21 ; CCC1(CC)c2ccc(N(c3cccc(C)c3)c3ccc4c(c3)C(C)(C)c3ccccc3-4)cc2-c2cc3ccccc3cc21 ; CCC1(CC)c2ccccc2-c2ccc(N(c3cccc(C)c3)c3ccc4c(c3)-c3cc5ccccc5cc3C4(C)C)cc21] 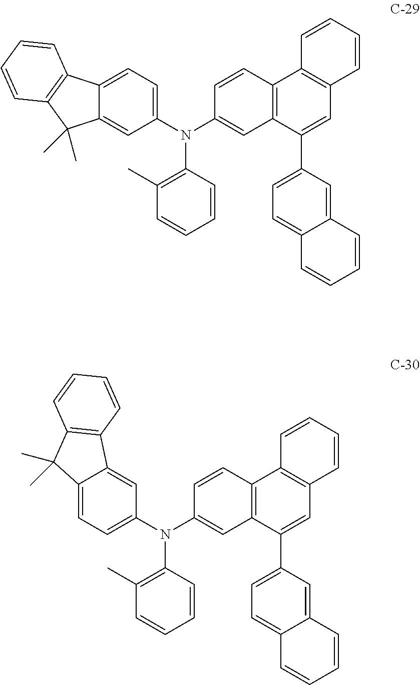 [SMILES: Cc1ccccc1N(c1ccc2c(c1)C(C)(C)c1ccccc1-2)c1ccc2c(c1)c(-c1ccc3ccccc3c1)cc1ccccc12 ; Cc1ccccc1N(c1ccc2c(c1)-c1ccccc1C2(C)C)c1ccc2c(c1)c(-c1ccc3ccccc3c1)cc1ccccc12] 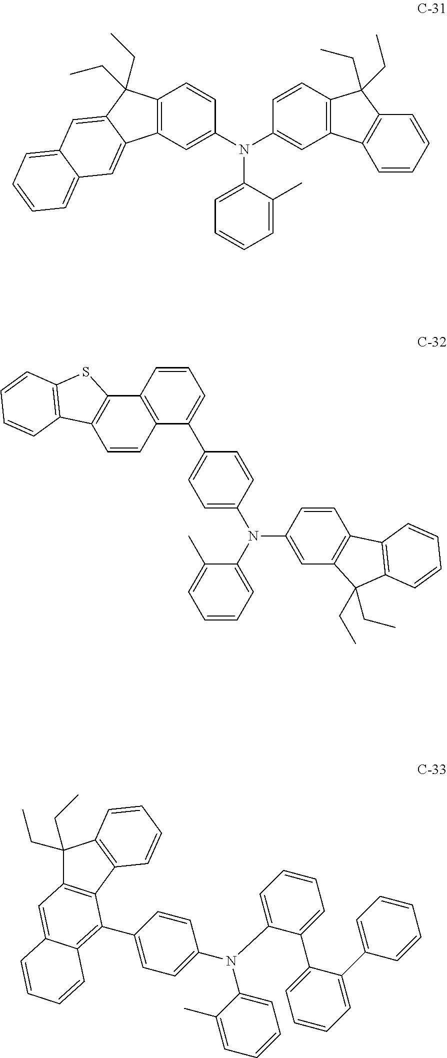 [SMILES: CCC1(CC)c2ccccc2-c2cc(N(c3ccc4c(c3)-c3cc5ccccc5cc3C4(CC)CC)c3ccccc3C)ccc21 ; CCC1(CC)c2ccccc2-c2ccc(N(c3ccc(-c4cccc5c4ccc4c6ccccc6sc54)cc3)c3ccccc3C)cc21 ; CCC1(CC)c2ccccc2-c2c1cc1ccccc1c2-c1ccc(N(c2ccccc2C)c2ccccc2-c2ccccc2-c2ccccc2)cc1] 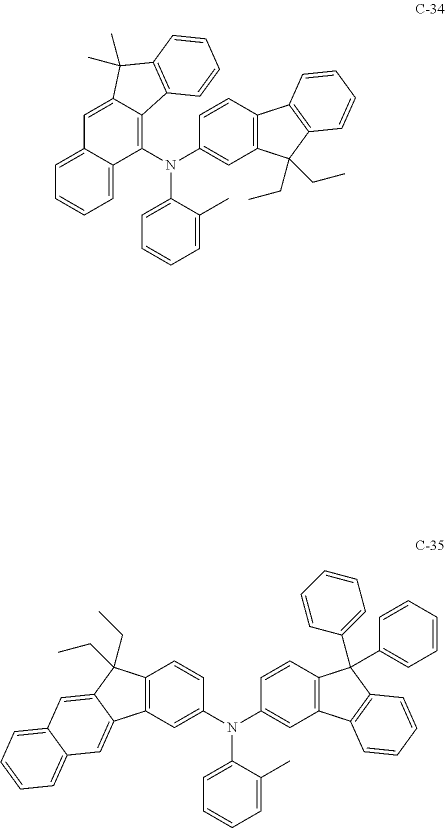 [SMILES: CCC1(CC)c2ccccc2-c2ccc(N(c3ccccc3C)c3c4c(cc5ccccc35)C(C)(C)c3ccccc3-4)cc21 ; CCC1(CC)c2ccc(N(c3ccc4c(c3)-c3ccccc3C4(c3ccccc3)c3ccccc3)c3ccccc3C)cc2-c2cc3ccccc3cc21] 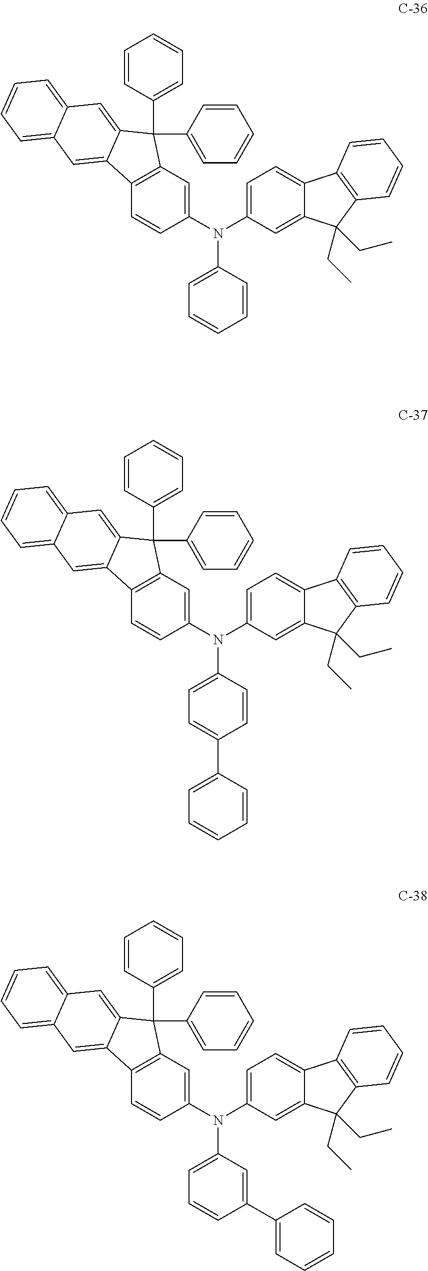 [SMILES: CCC1(CC)c2ccccc2-c2ccc(N(c3ccccc3)c3ccc4c(c3)C(c3ccccc3)(c3ccccc3)c3cc5ccccc5cc3-4)cc21 ; CCC1(CC)c2ccccc2-c2ccc(N(c3ccc(-c4ccccc4)cc3)c3ccc4c(c3)C(c3ccccc3)(c3ccccc3)c3cc5ccccc5cc3-4)cc21 ; CCC1(CC)c2ccccc2-c2ccc(N(c3cccc(-c4ccccc4)c3)c3ccc4c(c3)C(c3ccccc3)(c3ccccc3)c3cc5ccccc5cc3-4)cc21] 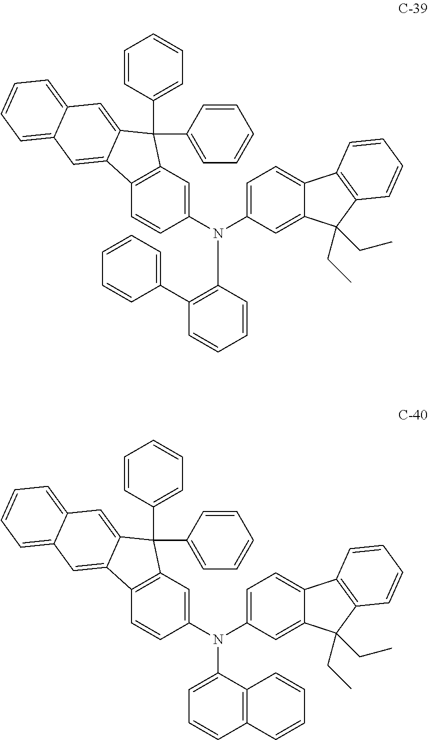 [SMILES: CCC1(CC)c2ccccc2-c2ccc(N(c3ccc4c(c3)C(c3ccccc3)(c3ccccc3)c3cc5ccccc5cc3-4)c3ccccc3-c3ccccc3)cc21 ; CCC1(CC)c2ccccc2-c2ccc(N(c3ccc4c(c3)C(c3ccccc3)(c3ccccc3)c3cc5ccccc5cc3-4)c3cccc4ccccc34)cc21] 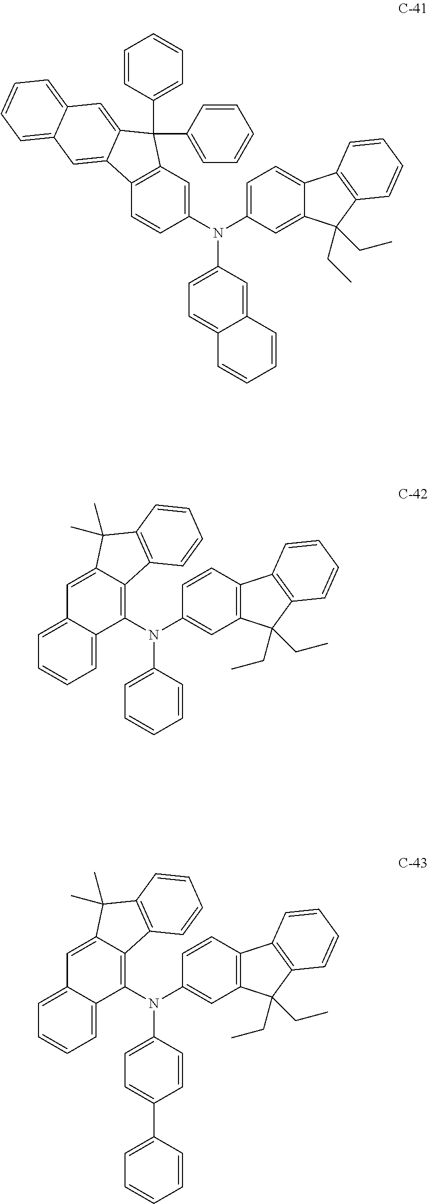 [SMILES: CCC1(CC)c2ccccc2-c2ccc(N(c3ccc4c(c3)C(c3ccccc3)(c3ccccc3)c3cc5ccccc5cc3-4)c3ccc4ccccc4c3)cc21 ; CCC1(CC)c2ccccc2-c2ccc(N(c3ccccc3)c3c4c(cc5ccccc35)C(C)(C)c3ccccc3-4)cc21 ; CCC1(CC)c2ccccc2-c2ccc(N(c3ccc(-c4ccccc4)cc3)c3c4c(cc5ccccc35)C(C)(C)c3ccccc3-4)cc21] 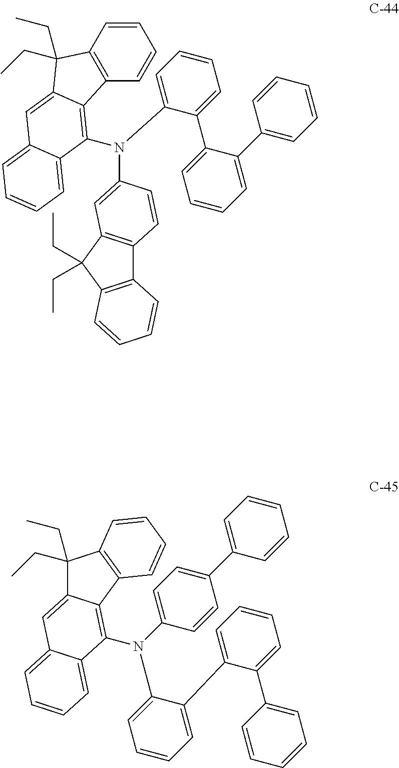 [SMILES: CCC1(CC)c2ccccc2-c2ccc(N(c3ccccc3-c3ccccc3-c3ccccc3)c3c4c(cc5ccccc35)C(CC)(CC)c3ccccc3-4)cc21 ; CCC1(CC)c2ccccc2-c2c1cc1ccccc1c2N(c1ccc(-c2ccccc2)cc1)c1ccccc1-c1ccccc1-c1ccccc1] 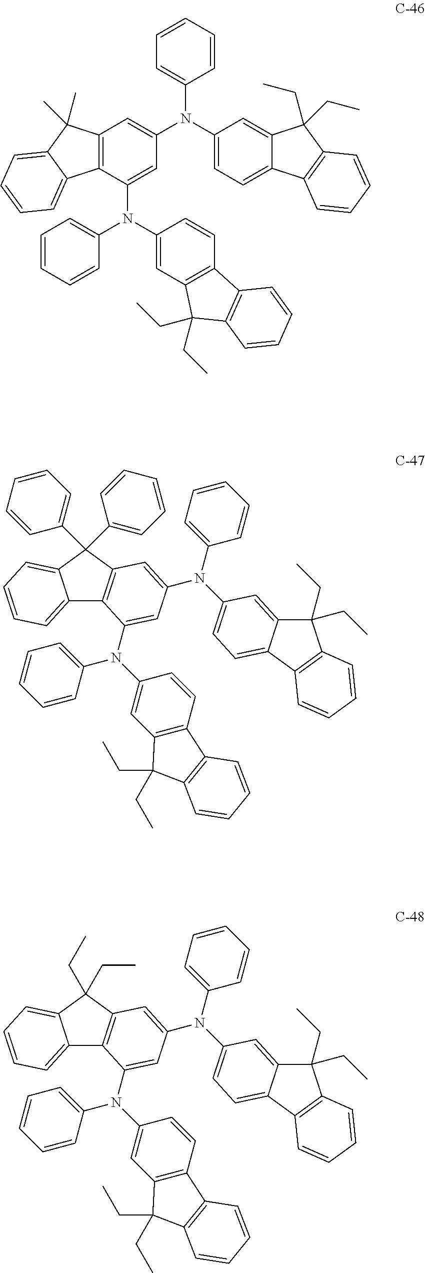 [SMILES: CCC1(CC)c2ccccc2-c2ccc(N(c3ccccc3)c3cc(N(c4ccccc4)c4ccc5c(c4)C(CC)(CC)c4ccccc4-5)c4c(c3)C(C)(C)c3ccccc3-4)cc21 ; CCC1(CC)c2ccccc2-c2ccc(N(c3ccccc3)c3cc(N(c4ccccc4)c4ccc5c(c4)C(CC)(CC)c4ccccc4-5)c4c(c3)C(c3ccccc3)(c3ccccc3)c3ccccc3-4)cc21 ; CCC1(CC)c2ccccc2-c2ccc(N(c3ccccc3)c3cc(N(c4ccccc4)c4ccc5c(c4)C(CC)(CC)c4ccccc4-5)c4c(c3)C(CC)(CC)c3ccccc3-4)cc21] 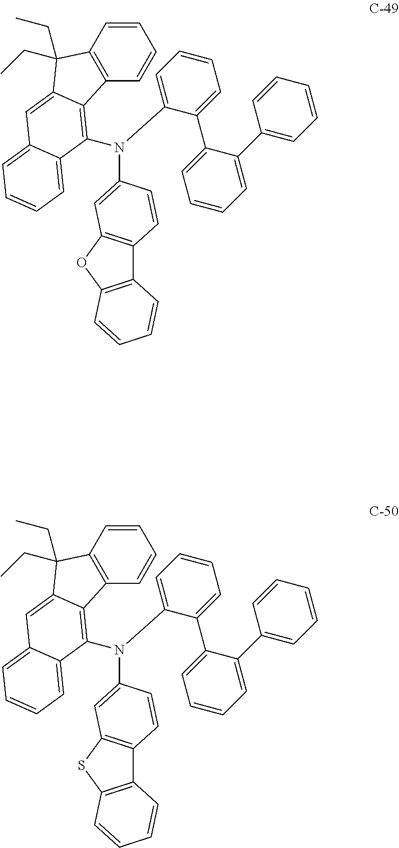 [SMILES: CCC1(CC)c2ccccc2-c2c1cc1ccccc1c2N(c1ccc2c(c1)oc1ccccc12)c1ccccc1-c1ccccc1-c1ccccc1 ; CCC1(CC)c2ccccc2-c2c1cc1ccccc1c2N(c1ccc2c(c1)sc1ccccc12)c1ccccc1-c1ccccc1-c1ccccc1] 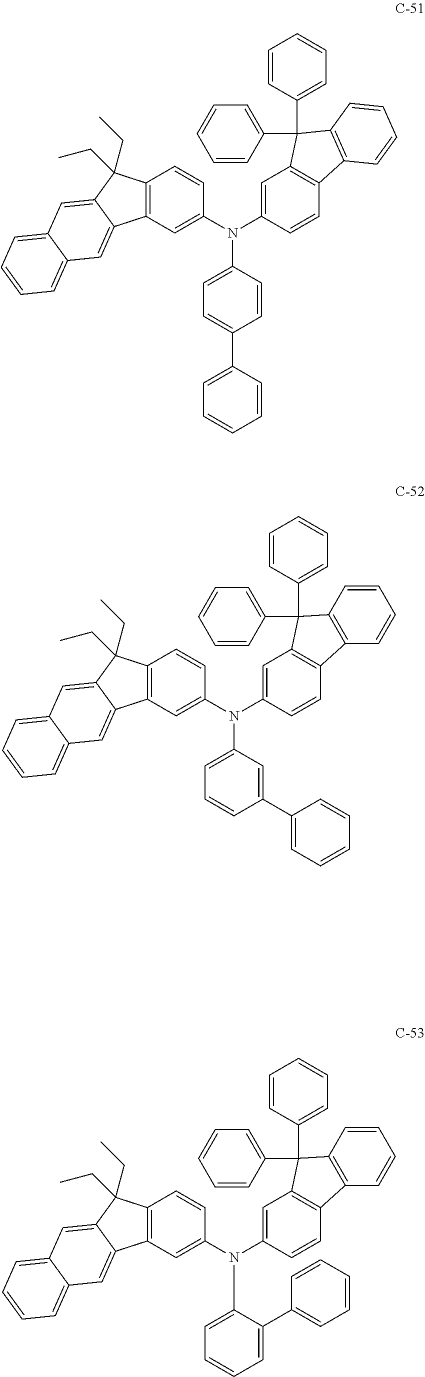 [SMILES: CCC1(CC)c2ccc(N(c3ccc(-c4ccccc4)cc3)c3ccc4c(c3)C(c3ccccc3)(c3ccccc3)c3ccccc3-4)cc2-c2cc3ccccc3cc21 ; CCC1(CC)c2ccc(N(c3cccc(-c4ccccc4)c3)c3ccc4c(c3)C(c3ccccc3)(c3ccccc3)c3ccccc3-4)cc2-c2cc3ccccc3cc21 ; CCC1(CC)c2ccc(N(c3ccc4c(c3)C(c3ccccc3)(c3ccccc3)c3ccccc3-4)c3ccccc3-c3ccccc3)cc2-c2cc3ccccc3cc21] 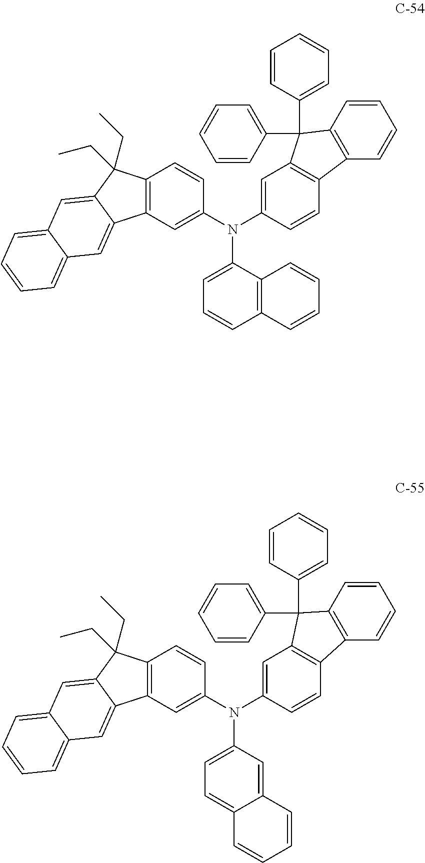 [SMILES: CCC1(CC)c2ccc(N(c3ccc4c(c3)C(c3ccccc3)(c3ccccc3)c3ccccc3-4)c3cccc4ccccc34)cc2-c2cc3ccccc3cc21 ; CCC1(CC)c2ccc(N(c3ccc4c(c3)C(c3ccccc3)(c3ccccc3)c3ccccc3-4)c3ccc4ccccc4c3)cc2-c2cc3ccccc3cc21] 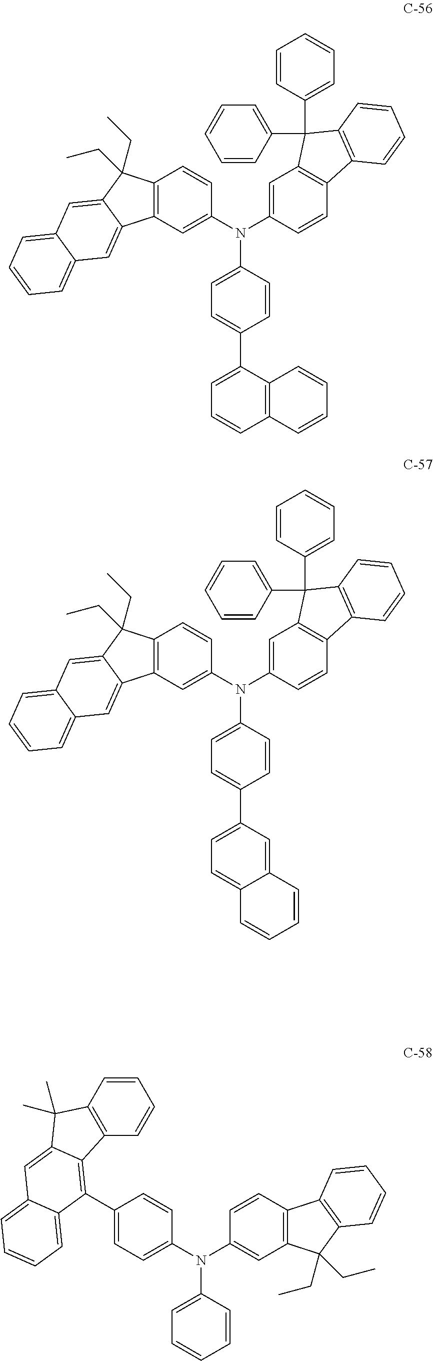 [SMILES: CCC1(CC)c2ccc(N(c3ccc(-c4cccc5ccccc45)cc3)c3ccc4c(c3)C(c3ccccc3)(c3ccccc3)c3ccccc3-4)cc2-c2cc3ccccc3cc21 ; CCC1(CC)c2ccc(N(c3ccc(-c4ccc5ccccc5c4)cc3)c3ccc4c(c3)C(c3ccccc3)(c3ccccc3)c3ccccc3-4)cc2-c2cc3ccccc3cc21 ; CCC1(CC)c2ccccc2-c2ccc(N(c3ccccc3)c3ccc(-c4c5c(cc6ccccc46)C(C)(C)c4ccccc4-5)cc3)cc21] 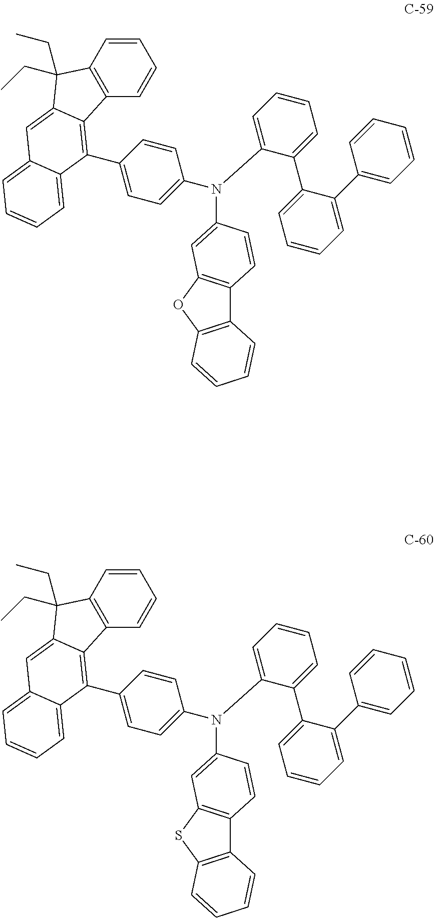 [SMILES: CCC1(CC)c2ccccc2-c2c1cc1ccccc1c2-c1ccc(N(c2ccc3c(c2)oc2ccccc23)c2ccccc2-c2ccccc2-c2ccccc2)cc1 ; CCC1(CC)c2ccccc2-c2c1cc1ccccc1c2-c1ccc(N(c2ccc3c(c2)sc2ccccc23)c2ccccc2-c2ccccc2-c2ccccc2)cc1] 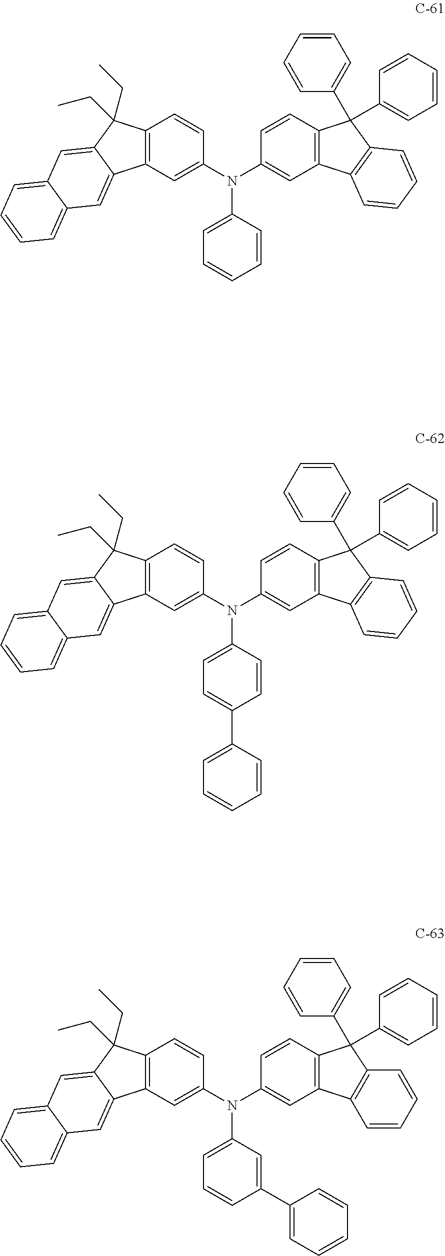 [SMILES: CCC1(CC)c2ccc(N(c3ccccc3)c3ccc4c(c3)-c3ccccc3C4(c3ccccc3)c3ccccc3)cc2-c2cc3ccccc3cc21 ; CCC1(CC)c2ccc(N(c3ccc(-c4ccccc4)cc3)c3ccc4c(c3)-c3ccccc3C4(c3ccccc3)c3ccccc3)cc2-c2cc3ccccc3cc21 ; CCC1(CC)c2ccc(N(c3cccc(-c4ccccc4)c3)c3ccc4c(c3)-c3ccccc3C4(c3ccccc3)c3ccccc3)cc2-c2cc3ccccc3cc21] 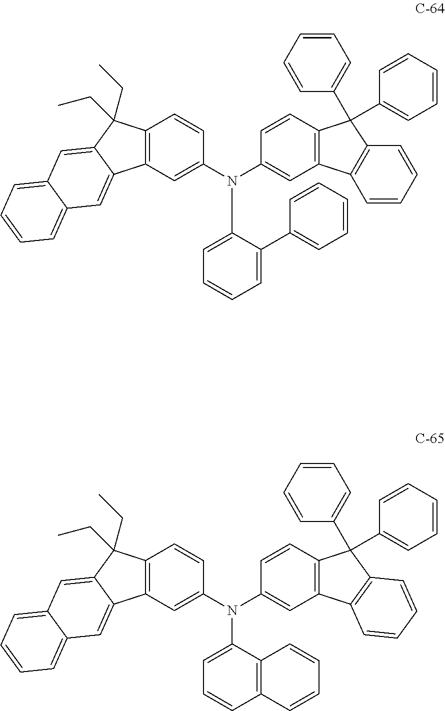 [SMILES: CCC1(CC)c2ccc(N(c3ccc4c(c3)-c3ccccc3C4(c3ccccc3)c3ccccc3)c3ccccc3-c3ccccc3)cc2-c2cc3ccccc3cc21 ; CCC1(CC)c2ccc(N(c3ccc4c(c3)-c3ccccc3C4(c3ccccc3)c3ccccc3)c3cccc4ccccc34)cc2-c2cc3ccccc3cc21] 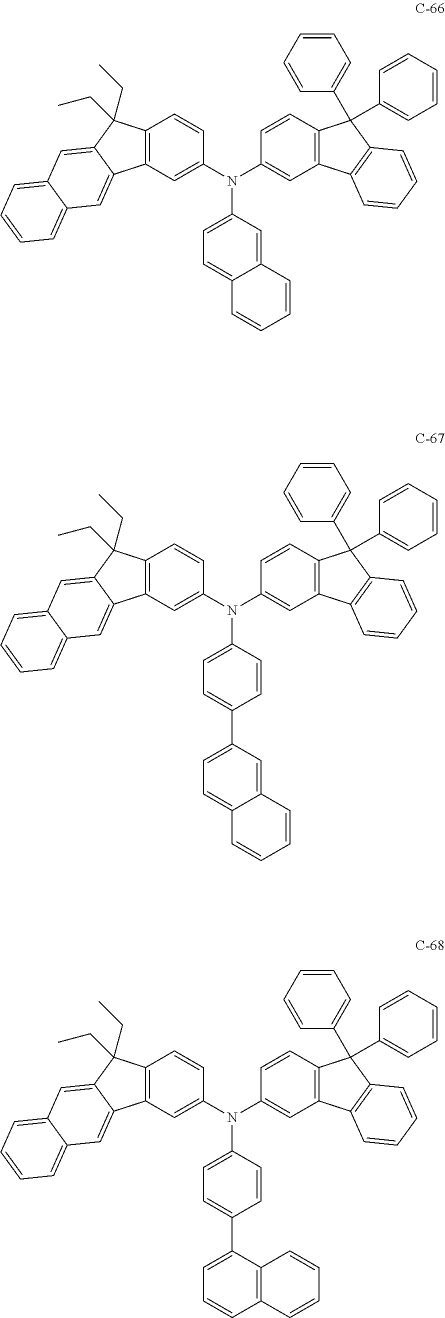 [SMILES: CCC1(CC)c2ccc(N(c3ccc4c(c3)-c3ccccc3C4(c3ccccc3)c3ccccc3)c3ccc4ccccc4c3)cc2-c2cc3ccccc3cc21 ; CCC1(CC)c2ccc(N(c3ccc(-c4ccc5ccccc5c4)cc3)c3ccc4c(c3)-c3ccccc3C4(c3ccccc3)c3ccccc3)cc2-c2cc3ccccc3cc21 ; CCC1(CC)c2ccc(N(c3ccc(-c4cccc5ccccc45)cc3)c3ccc4c(c3)-c3ccccc3C4(c3ccccc3)c3ccccc3)cc2-c2cc3ccccc3cc21] 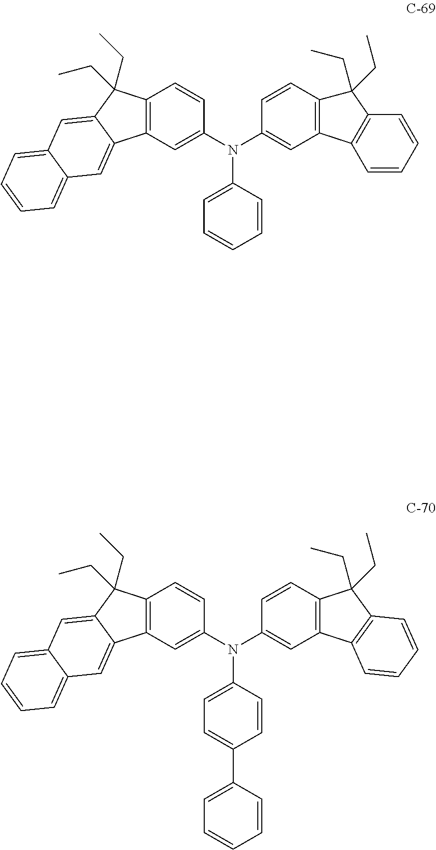 [SMILES: CCC1(CC)c2ccccc2-c2cc(N(c3ccccc3)c3ccc4c(c3)-c3cc5ccccc5cc3C4(CC)CC)ccc21 ; CCC1(CC)c2ccccc2-c2cc(N(c3ccc(-c4ccccc4)cc3)c3ccc4c(c3)-c3cc5ccccc5cc3C4(CC)CC)ccc21] 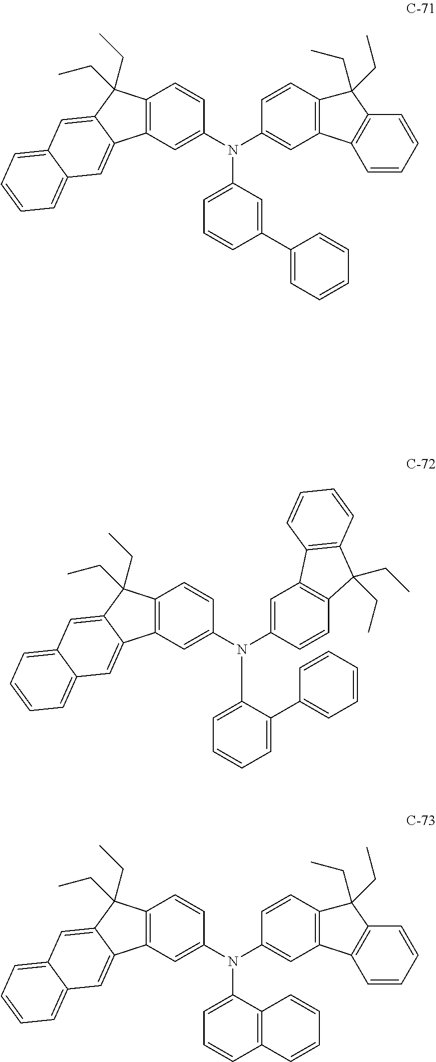 [SMILES: CCC1(CC)c2ccccc2-c2cc(N(c3cccc(-c4ccccc4)c3)c3ccc4c(c3)-c3cc5ccccc5cc3C4(CC)CC)ccc21 ; CCC1(CC)c2ccccc2-c2cc(N(c3ccc4c(c3)-c3cc5ccccc5cc3C4(CC)CC)c3ccccc3-c3ccccc3)ccc21 ; CCC1(CC)c2ccccc2-c2cc(N(c3ccc4c(c3)-c3cc5ccccc5cc3C4(CC)CC)c3cccc4ccccc34)ccc21] 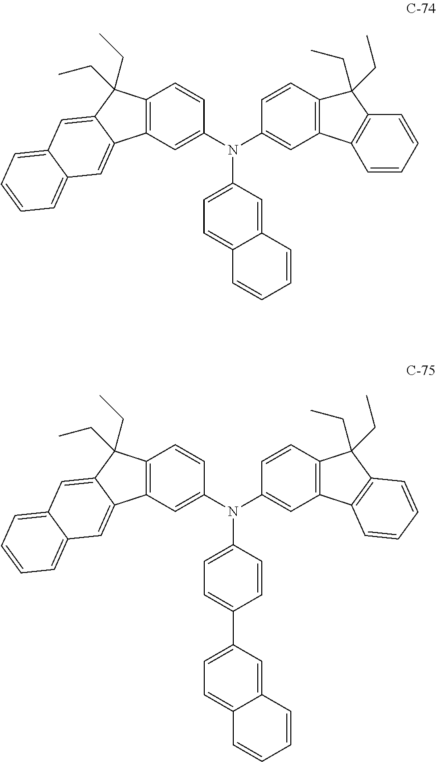 [SMILES: CCC1(CC)c2ccccc2-c2cc(N(c3ccc4c(c3)-c3cc5ccccc5cc3C4(CC)CC)c3ccc4ccccc4c3)ccc21 ; CCC1(CC)c2ccccc2-c2cc(N(c3ccc(-c4ccc5ccccc5c4)cc3)c3ccc4c(c3)-c3cc5ccccc5cc3C4(CC)CC)ccc21] 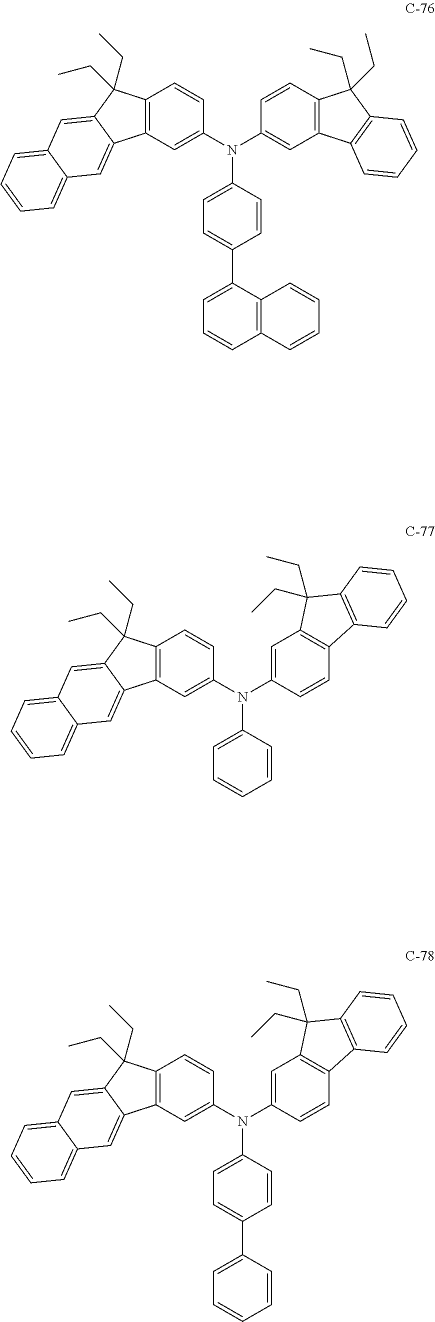 [SMILES: CCC1(CC)c2ccccc2-c2cc(N(c3ccc(-c4cccc5ccccc45)cc3)c3ccc4c(c3)-c3cc5ccccc5cc3C4(CC)CC)ccc21 ; CCC1(CC)c2ccccc2-c2ccc(N(c3ccccc3)c3ccc4c(c3)-c3cc5ccccc5cc3C4(CC)CC)cc21 ; CCC1(CC)c2ccccc2-c2ccc(N(c3ccc(-c4ccccc4)cc3)c3ccc4c(c3)-c3cc5ccccc5cc3C4(CC)CC)cc21] 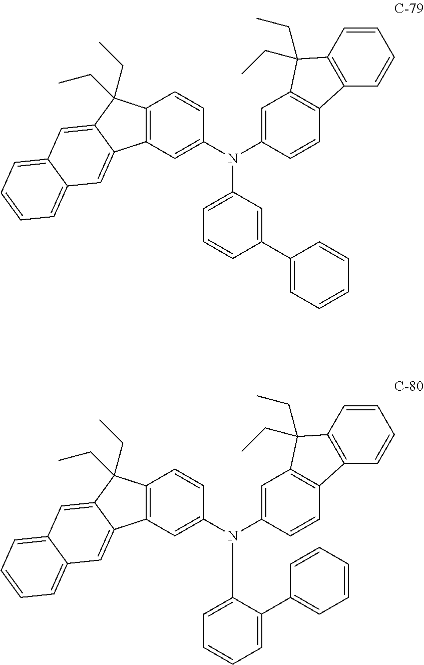 [SMILES: CCC1(CC)c2ccccc2-c2ccc(N(c3cccc(-c4ccccc4)c3)c3ccc4c(c3)-c3cc5ccccc5cc3C4(CC)CC)cc21 ; CCC1(CC)c2ccccc2-c2ccc(N(c3ccc4c(c3)-c3cc5ccccc5cc3C4(CC)CC)c3ccccc3-c3ccccc3)cc21] 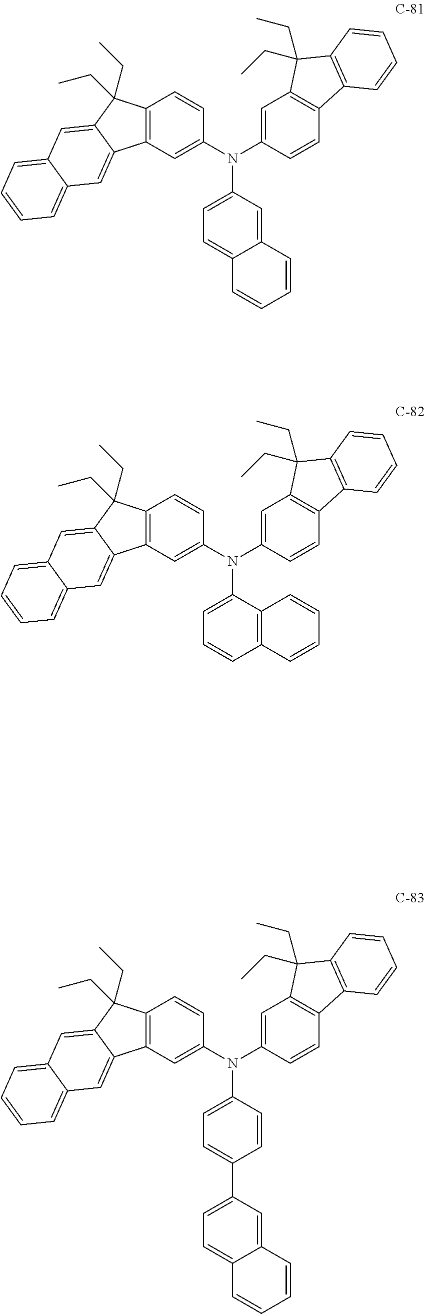 [SMILES: CCC1(CC)c2ccccc2-c2ccc(N(c3ccc4c(c3)-c3cc5ccccc5cc3C4(CC)CC)c3ccc4ccccc4c3)cc21 ; CCC1(CC)c2ccccc2-c2ccc(N(c3ccc4c(c3)-c3cc5ccccc5cc3C4(CC)CC)c3cccc4ccccc34)cc21 ; CCC1(CC)c2ccccc2-c2ccc(N(c3ccc(-c4ccc5ccccc5c4)cc3)c3ccc4c(c3)-c3cc5ccccc5cc3C4(CC)CC)cc21] 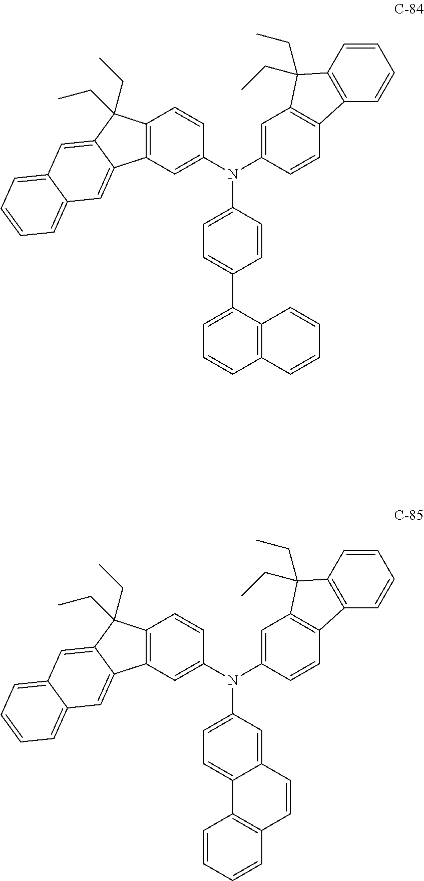 [SMILES: CCC1(CC)c2ccccc2-c2ccc(N(c3ccc(-c4cccc5ccccc45)cc3)c3ccc4c(c3)-c3cc5ccccc5cc3C4(CC)CC)cc21 ; CCC1(CC)c2ccccc2-c2ccc(N(c3ccc4c(c3)-c3cc5ccccc5cc3C4(CC)CC)c3ccc4c(ccc5ccccc54)c3)cc21] 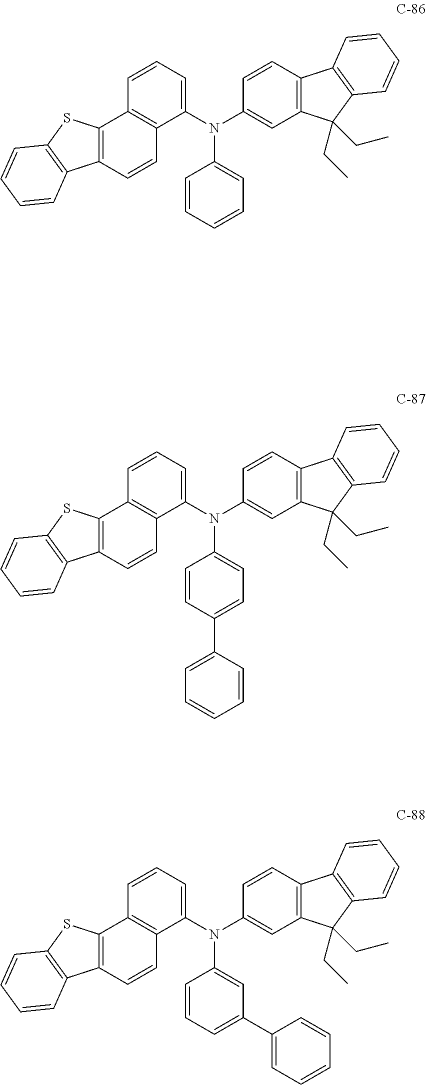 [SMILES: CCC1(CC)c2ccccc2-c2ccc(N(c3ccccc3)c3cccc4c3ccc3c5ccccc5sc43)cc21 ; CCC1(CC)c2ccccc2-c2ccc(N(c3ccc(-c4ccccc4)cc3)c3cccc4c3ccc3c5ccccc5sc43)cc21 ; CCC1(CC)c2ccccc2-c2ccc(N(c3cccc(-c4ccccc4)c3)c3cccc4c3ccc3c5ccccc5sc43)cc21] 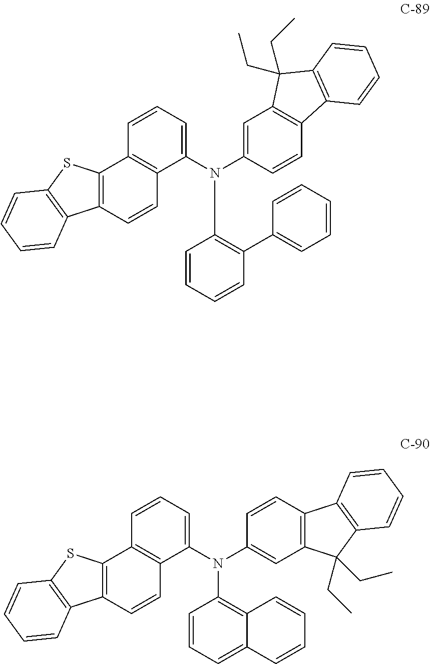 [SMILES: CCC1(CC)c2ccccc2-c2ccc(N(c3ccccc3-c3ccccc3)c3cccc4c3ccc3c5ccccc5sc43)cc21 ; CCC1(CC)c2ccccc2-c2ccc(N(c3cccc4ccccc34)c3cccc4c3ccc3c5ccccc5sc43)cc21] 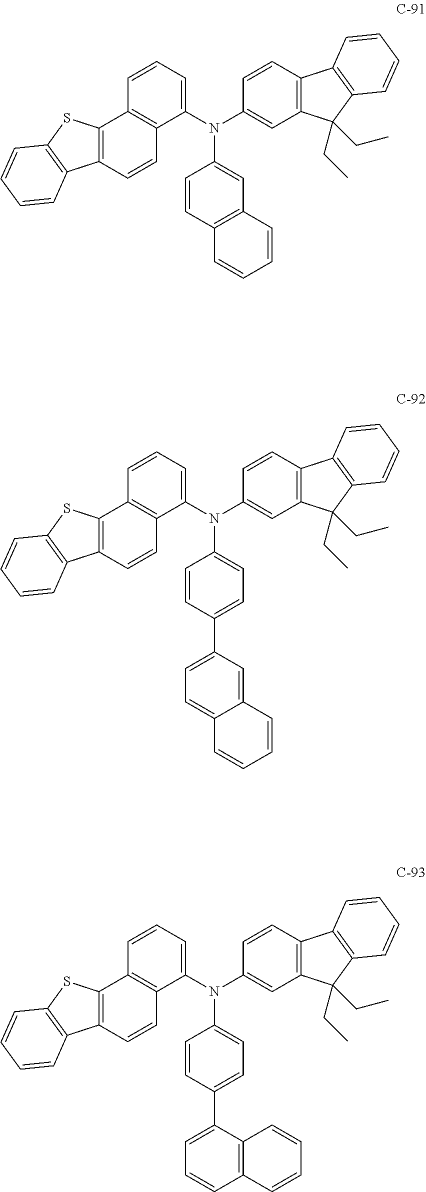 [SMILES: CCC1(CC)c2ccccc2-c2ccc(N(c3ccc4ccccc4c3)c3cccc4c3ccc3c5ccccc5sc43)cc21 ; CCC1(CC)c2ccccc2-c2ccc(N(c3ccc(-c4ccc5ccccc5c4)cc3)c3cccc4c3ccc3c5ccccc5sc43)cc21 ; CCC1(CC)c2ccccc2-c2ccc(N(c3ccc(-c4cccc5ccccc45)cc3)c3cccc4c3ccc3c5ccccc5sc43)cc21] 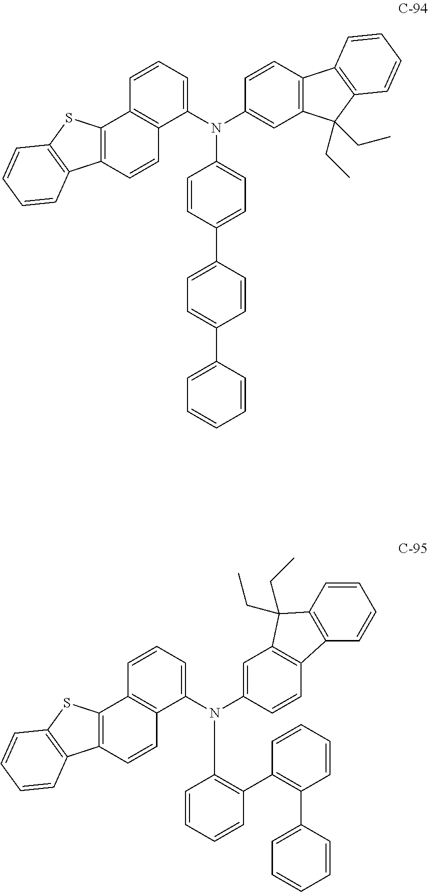 [SMILES: CCC1(CC)c2ccccc2-c2ccc(N(c3ccc(-c4ccc(-c5ccccc5)cc4)cc3)c3cccc4c3ccc3c5ccccc5sc43)cc21 ; CCC1(CC)c2ccccc2-c2ccc(N(c3ccccc3-c3ccccc3-c3ccccc3)c3cccc4c3ccc3c5ccccc5sc43)cc21] 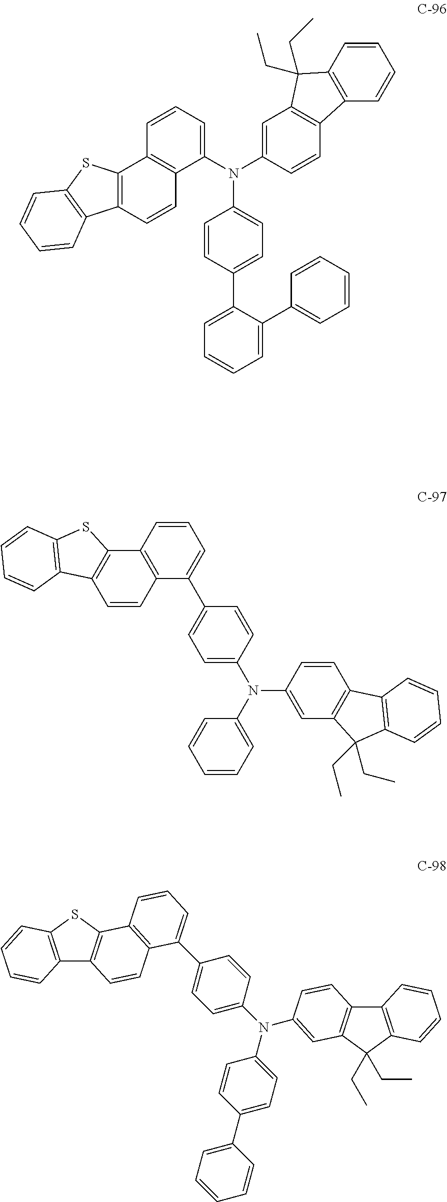 [SMILES: CCC1(CC)c2ccccc2-c2ccc(N(c3ccc(-c4ccccc4-c4ccccc4)cc3)c3cccc4c3ccc3c5ccccc5sc43)cc21 ; CCC1(CC)c2ccccc2-c2ccc(N(c3ccccc3)c3ccc(-c4cccc5c4ccc4c6ccccc6sc54)cc3)cc21 ; CCC1(CC)c2ccccc2-c2ccc(N(c3ccc(-c4ccccc4)cc3)c3ccc(-c4cccc5c4ccc4c6ccccc6sc54)cc3)cc21] 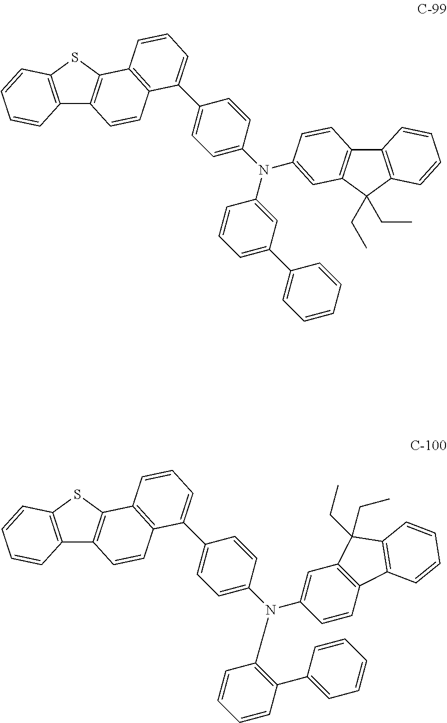 [SMILES: CCC1(CC)c2ccccc2-c2ccc(N(c3ccc(-c4cccc5c4ccc4c6ccccc6sc54)cc3)c3cccc(-c4ccccc4)c3)cc21 ; CCC1(CC)c2ccccc2-c2ccc(N(c3ccc(-c4cccc5c4ccc4c6ccccc6sc54)cc3)c3ccccc3-c3ccccc3)cc21] 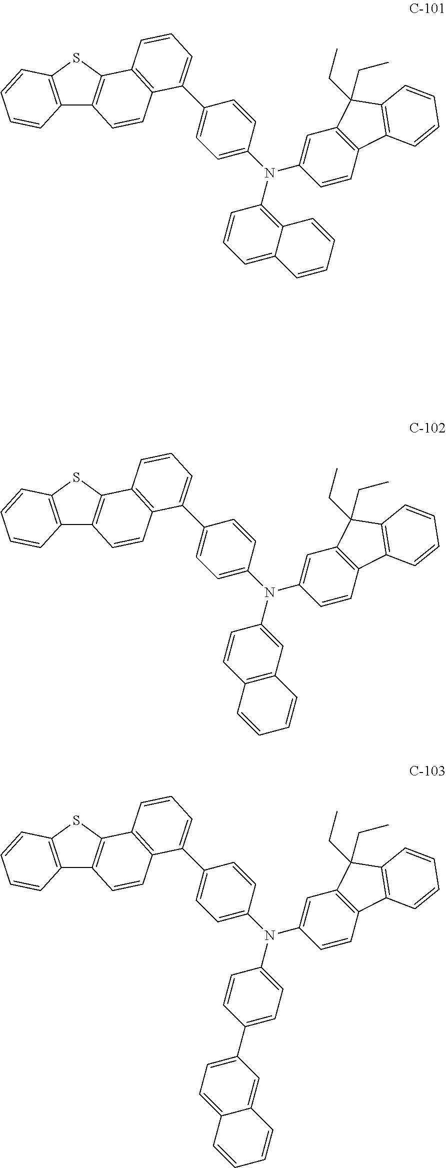 [SMILES: CCC1(CC)c2ccccc2-c2ccc(N(c3ccc(-c4cccc5c4ccc4c6ccccc6sc54)cc3)c3cccc4ccccc34)cc21 ; CCC1(CC)c2ccccc2-c2ccc(N(c3ccc(-c4cccc5c4ccc4c6ccccc6sc54)cc3)c3ccc4ccccc4c3)cc21 ; CCC1(CC)c2ccccc2-c2ccc(N(c3ccc(-c4ccc5ccccc5c4)cc3)c3ccc(-c4cccc5c4ccc4c6ccccc6sc54)cc3)cc21] 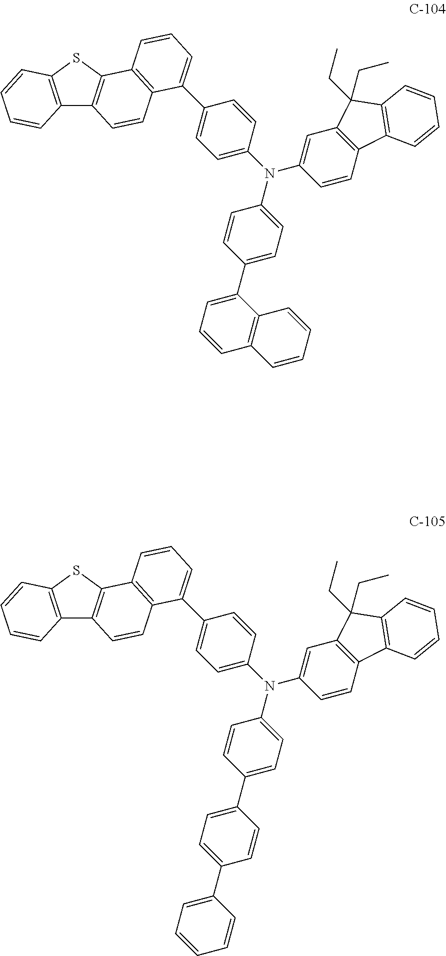 [SMILES: CCC1(CC)c2ccccc2-c2ccc(N(c3ccc(-c4cccc5ccccc45)cc3)c3ccc(-c4cccc5c4ccc4c6ccccc6sc54)cc3)cc21 ; CCC1(CC)c2ccccc2-c2ccc(N(c3ccc(-c4ccc(-c5ccccc5)cc4)cc3)c3ccc(-c4cccc5c4ccc4c6ccccc6sc54)cc3)cc21] 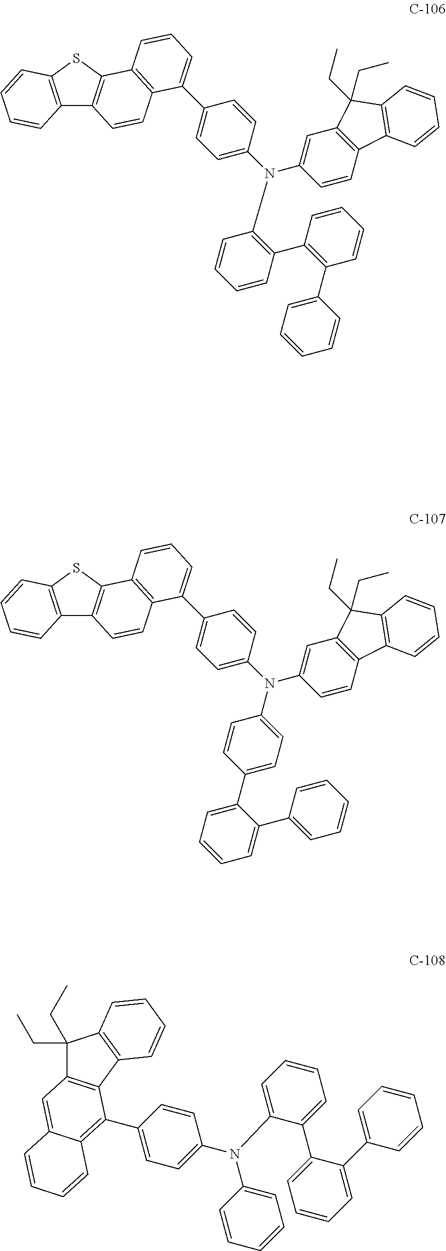 [SMILES: CCC1(CC)c2ccccc2-c2ccc(N(c3ccc(-c4cccc5c4ccc4c6ccccc6sc54)cc3)c3ccccc3-c3ccccc3-c3ccccc3)cc21 ; CCC1(CC)c2ccccc2-c2ccc(N(c3ccc(-c4ccccc4-c4ccccc4)cc3)c3ccc(-c4cccc5c4ccc4c6ccccc6sc54)cc3)cc21 ; CCC1(CC)c2ccccc2-c2c1cc1ccccc1c2-c1ccc(N(c2ccccc2)c2ccccc2-c2ccccc2-c2ccccc2)cc1] 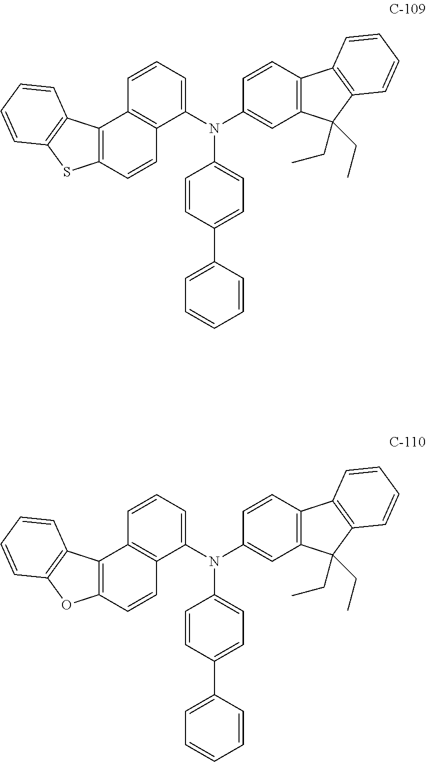 [SMILES: CCC1(CC)c2ccccc2-c2ccc(N(c3ccc(-c4ccccc4)cc3)c3cccc4c3ccc3sc5ccccc5c34)cc21 ; CCC1(CC)c2ccccc2-c2ccc(N(c3ccc(-c4ccccc4)cc3)c3cccc4c3ccc3oc5ccccc5c34)cc21] 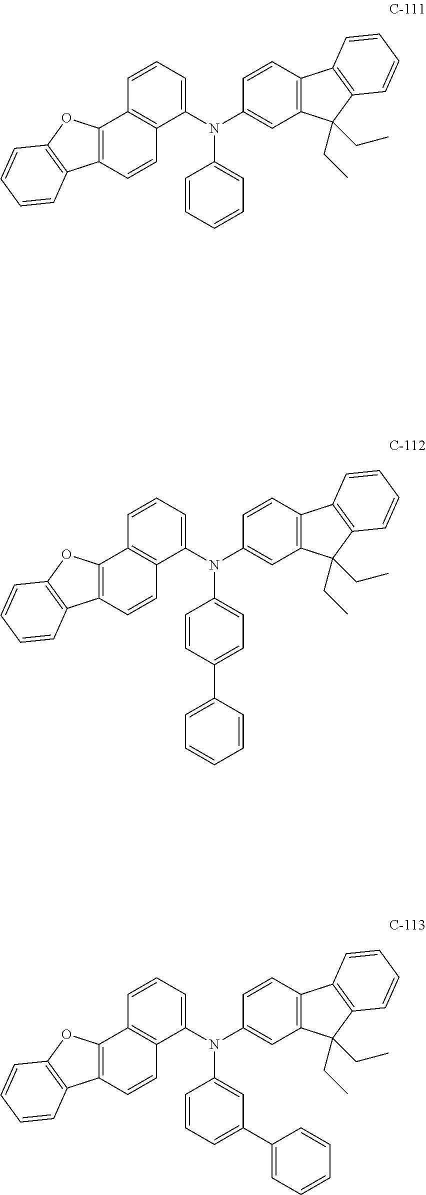 [SMILES: CCC1(CC)c2ccccc2-c2ccc(N(c3ccccc3)c3cccc4c3ccc3c5ccccc5oc43)cc21 ; CCC1(CC)c2ccccc2-c2ccc(N(c3ccc(-c4ccccc4)cc3)c3cccc4c3ccc3c5ccccc5oc43)cc21 ; CCC1(CC)c2ccccc2-c2ccc(N(c3cccc(-c4ccccc4)c3)c3cccc4c3ccc3c5ccccc5oc43)cc21] 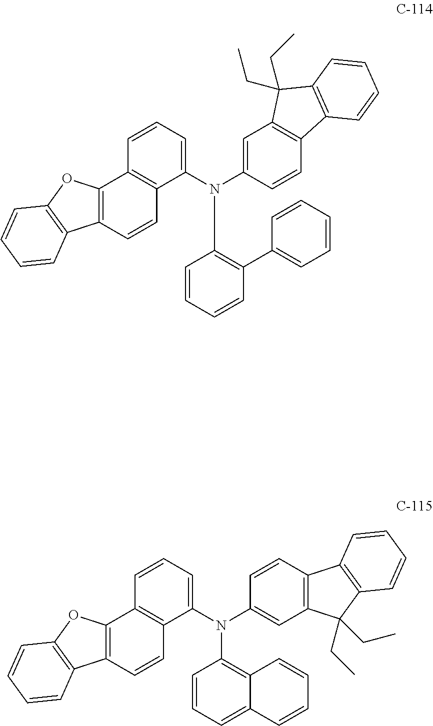 [SMILES: CCC1(CC)c2ccccc2-c2ccc(N(c3ccccc3-c3ccccc3)c3cccc4c3ccc3c5ccccc5oc43)cc21 ; CCC1(CC)c2ccccc2-c2ccc(N(c3cccc4ccccc34)c3cccc4c3ccc3c5ccccc5oc43)cc21] 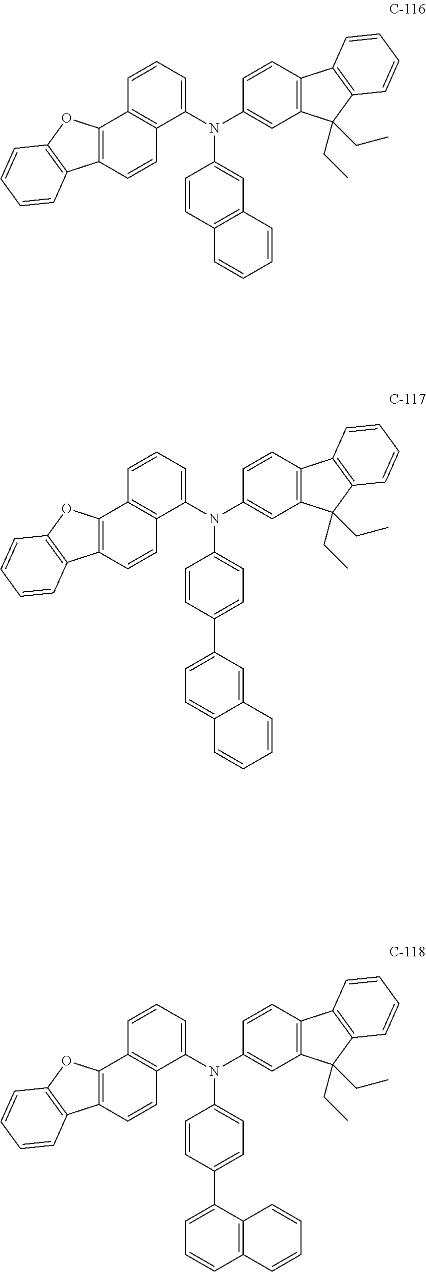 [SMILES: CCC1(CC)c2ccccc2-c2ccc(N(c3ccc4ccccc4c3)c3cccc4c3ccc3c5ccccc5oc43)cc21 ; CCC1(CC)c2ccccc2-c2ccc(N(c3ccc(-c4ccc5ccccc5c4)cc3)c3cccc4c3ccc3c5ccccc5oc43)cc21 ; CCC1(CC)c2ccccc2-c2ccc(N(c3ccc(-c4cccc5ccccc45)cc3)c3cccc4c3ccc3c5ccccc5oc43)cc21] 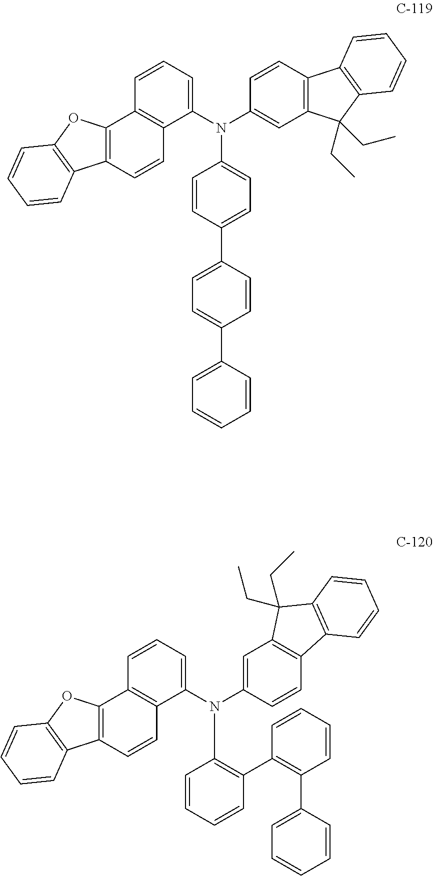 [SMILES: CCC1(CC)c2ccccc2-c2ccc(N(c3ccc(-c4ccc(-c5ccccc5)cc4)cc3)c3cccc4c3ccc3c5ccccc5oc43)cc21 ; CCC1(CC)c2ccccc2-c2ccc(N(c3ccccc3-c3ccccc3-c3ccccc3)c3cccc4c3ccc3c5ccccc5oc43)cc21] 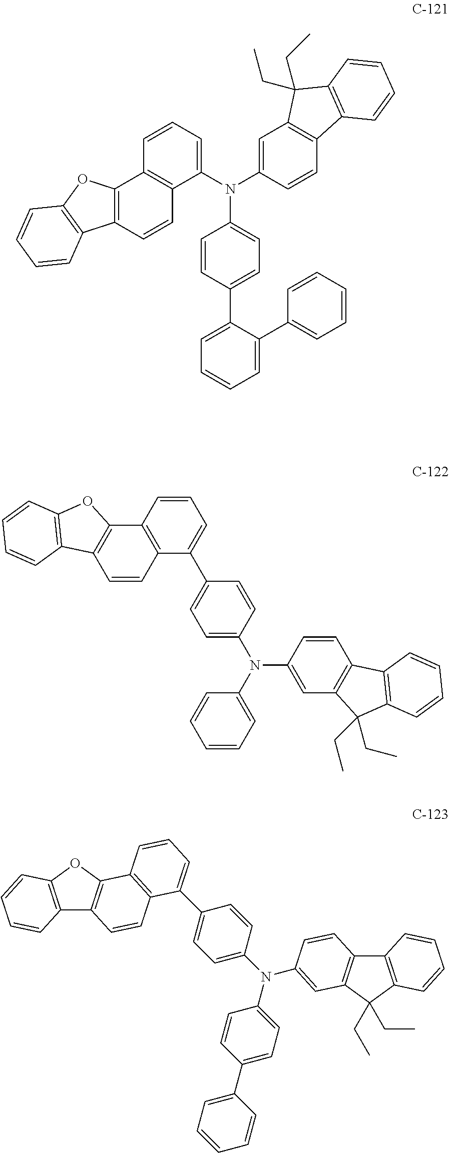 [SMILES: CCC1(CC)c2ccccc2-c2ccc(N(c3ccc(-c4ccccc4-c4ccccc4)cc3)c3cccc4c3ccc3c5ccccc5oc43)cc21 ; CCC1(CC)c2ccccc2-c2ccc(N(c3ccccc3)c3ccc(-c4cccc5c4ccc4c6ccccc6oc54)cc3)cc21 ; CCC1(CC)c2ccccc2-c2ccc(N(c3ccc(-c4ccccc4)cc3)c3ccc(-c4cccc5c4ccc4c6ccccc6oc54)cc3)cc21] 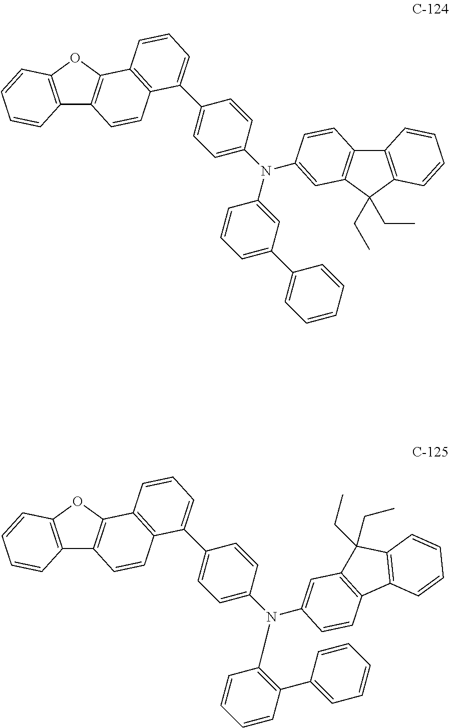 [SMILES: CCC1(CC)c2ccccc2-c2ccc(N(c3ccc(-c4cccc5c4ccc4c6ccccc6oc54)cc3)c3cccc(-c4ccccc4)c3)cc21 ; CCC1(CC)c2ccccc2-c2ccc(N(c3ccc(-c4cccc5c4ccc4c6ccccc6oc54)cc3)c3ccccc3-c3ccccc3)cc21] 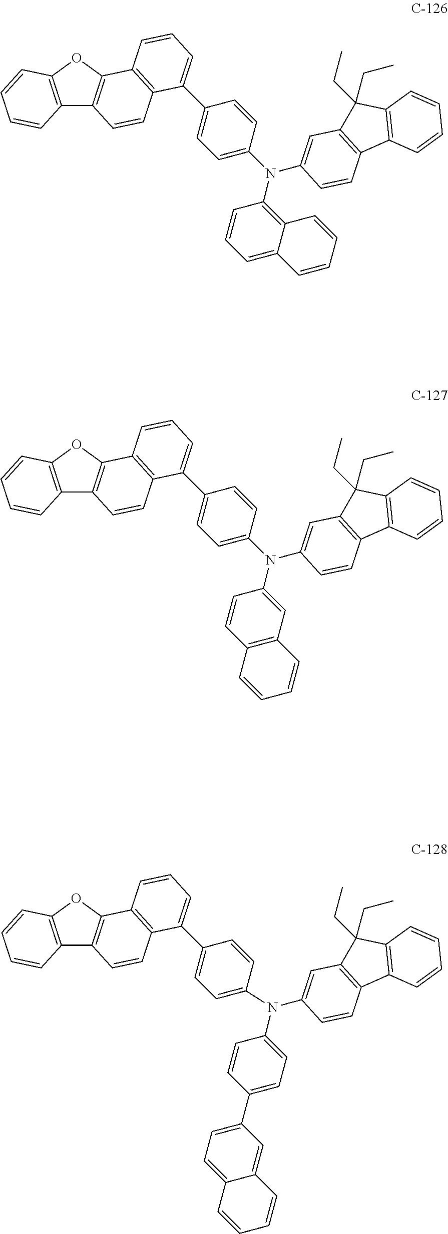 [SMILES: CCC1(CC)c2ccccc2-c2ccc(N(c3ccc(-c4cccc5c4ccc4c6ccccc6oc54)cc3)c3cccc4ccccc34)cc21 ; CCC1(CC)c2ccccc2-c2ccc(N(c3ccc(-c4cccc5c4ccc4c6ccccc6oc54)cc3)c3ccc4ccccc4c3)cc21 ; CCC1(CC)c2ccccc2-c2ccc(N(c3ccc(-c4ccc5ccccc5c4)cc3)c3ccc(-c4cccc5c4ccc4c6ccccc6oc54)cc3)cc21] 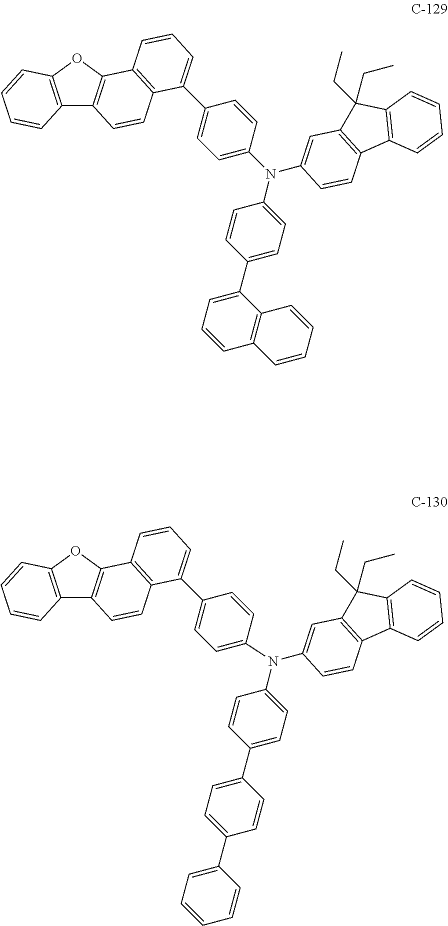 [SMILES: CCC1(CC)c2ccccc2-c2ccc(N(c3ccc(-c4cccc5ccccc45)cc3)c3ccc(-c4cccc5c4ccc4c6ccccc6oc54)cc3)cc21 ; CCC1(CC)c2ccccc2-c2ccc(N(c3ccc(-c4ccc(-c5ccccc5)cc4)cc3)c3ccc(-c4cccc5c4ccc4c6ccccc6oc54)cc3)cc21] 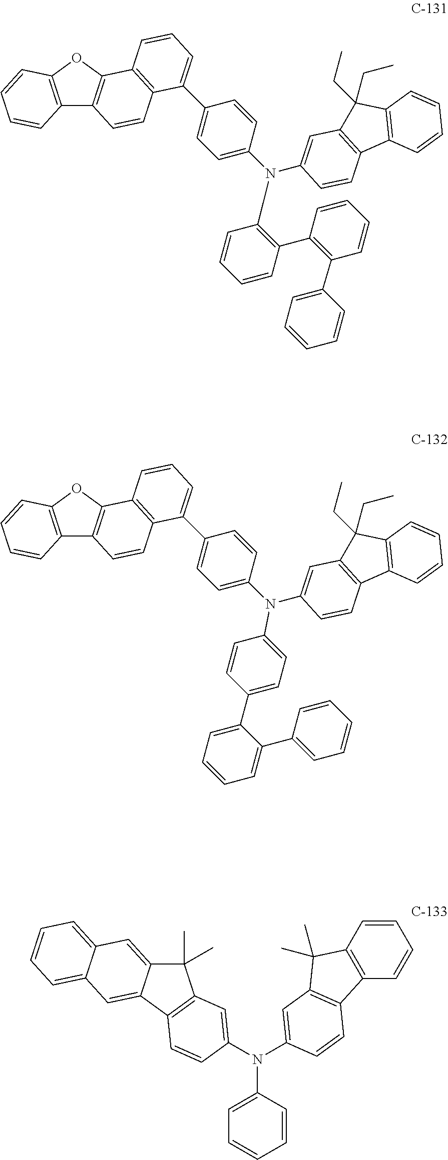 [SMILES: CCC1(CC)c2ccccc2-c2ccc(N(c3ccc(-c4cccc5c4ccc4c6ccccc6oc54)cc3)c3ccccc3-c3ccccc3-c3ccccc3)cc21 ; CCC1(CC)c2ccccc2-c2ccc(N(c3ccc(-c4ccccc4-c4ccccc4)cc3)c3ccc(-c4cccc5c4ccc4c6ccccc6oc54)cc3)cc21 ; CC1(C)c2ccccc2-c2ccc(N(c3ccccc3)c3ccc4c(c3)C(C)(C)c3cc5ccccc5cc3-4)cc21] 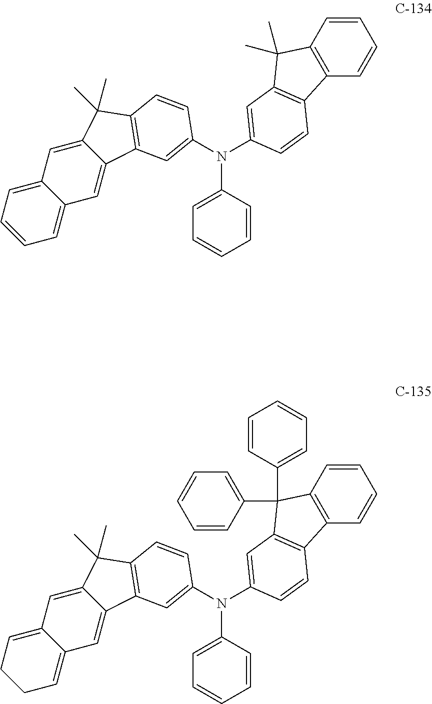 [SMILES: CC1(C)c2ccccc2-c2ccc(N(c3ccccc3)c3ccc4c(c3)-c3cc5ccccc5cc3C4(C)C)cc21 ; CC1(C)c2ccc(N(c3ccccc3)c3ccc4c(c3)C(c3ccccc3)(c3ccccc3)c3ccccc3-4)cc2-c2cc3c(cc21)=CCCC=3] 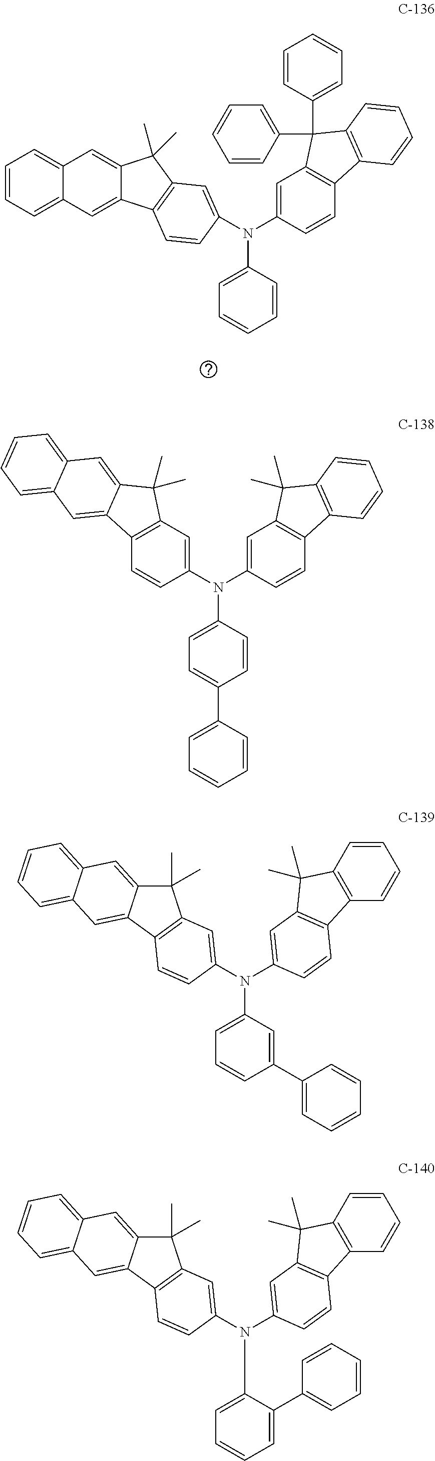 [SMILES: CC1(C)c2cc(N(c3ccccc3)c3ccc4c(c3)C(c3ccccc3)(c3ccccc3)c3ccccc3-4)ccc2-c2cc3ccccc3cc21 ; CC1(C)c2ccccc2-c2ccc(N(c3ccc(-c4ccccc4)cc3)c3ccc4c(c3)C(C)(C)c3cc5ccccc5cc3-4)cc21 ; CC1(C)c2ccccc2-c2ccc(N(c3cccc(-c4ccccc4)c3)c3ccc4c(c3)C(C)(C)c3cc5ccccc5cc3-4)cc21 ; CC1(C)c2ccccc2-c2ccc(N(c3ccc4c(c3)C(C)(C)c3cc5ccccc5cc3-4)c3ccccc3-c3ccccc3)cc21] 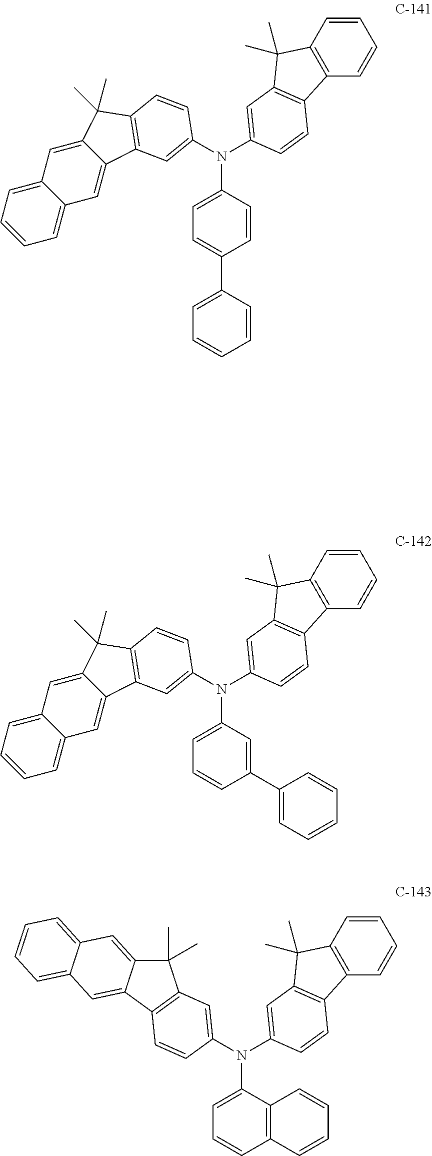 [SMILES: CC1(C)c2ccccc2-c2ccc(N(c3ccc(-c4ccccc4)cc3)c3ccc4c(c3)-c3cc5ccccc5cc3C4(C)C)cc21 ; CC1(C)c2ccccc2-c2ccc(N(c3cccc(-c4ccccc4)c3)c3ccc4c(c3)-c3cc5ccccc5cc3C4(C)C)cc21 ; CC1(C)c2ccccc2-c2ccc(N(c3ccc4c(c3)C(C)(C)c3cc5ccccc5cc3-4)c3cccc4ccccc34)cc21] 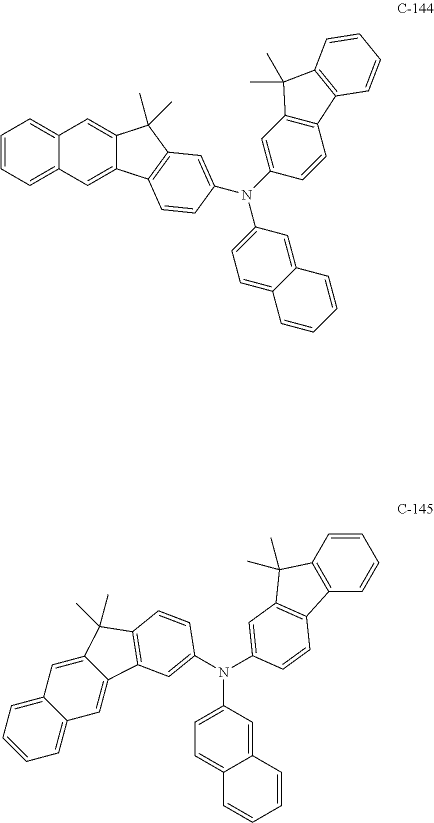 [SMILES: CC1(C)c2ccccc2-c2ccc(N(c3ccc4c(c3)C(C)(C)c3cc5ccccc5cc3-4)c3ccc4ccccc4c3)cc21 ; CC1(C)c2ccccc2-c2ccc(N(c3ccc4c(c3)-c3cc5ccccc5cc3C4(C)C)c3ccc4ccccc4c3)cc21] 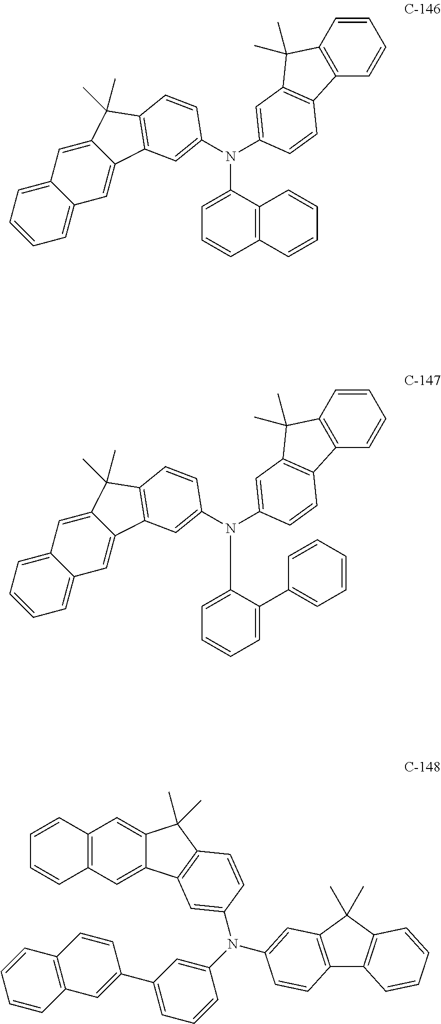 [SMILES: CC1(C)c2ccccc2-c2ccc(N(c3ccc4c(c3)-c3cc5ccccc5cc3C4(C)C)c3cccc4ccccc34)cc21 ; CC1(C)c2ccccc2-c2ccc(N(c3ccc4c(c3)-c3cc5ccccc5cc3C4(C)C)c3ccccc3-c3ccccc3)cc21 ; CC1(C)c2ccccc2-c2ccc(N(c3cccc(-c4ccc5ccccc5c4)c3)c3ccc4c(c3)-c3cc5ccccc5cc3C4(C)C)cc21] 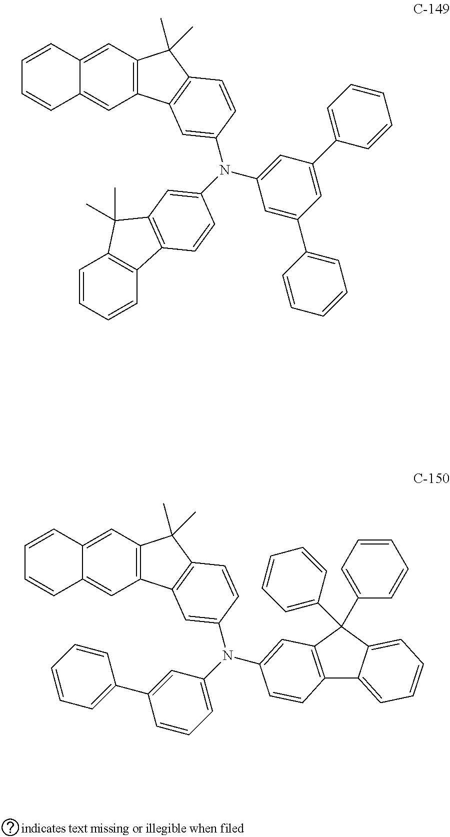 [SMILES: CC1(C)c2ccccc2-c2ccc(N(c3cc(-c4ccccc4)cc(-c4ccccc4)c3)c3ccc4c(c3)-c3cc5ccccc5cc3C4(C)C)cc21 ; CC1(C)c2ccc(N(c3cccc(-c4ccccc4)c3)c3ccc4c(c3)C(c3ccccc3)(c3ccccc3)c3ccccc3-4)cc2-c2cc3ccccc3cc21] 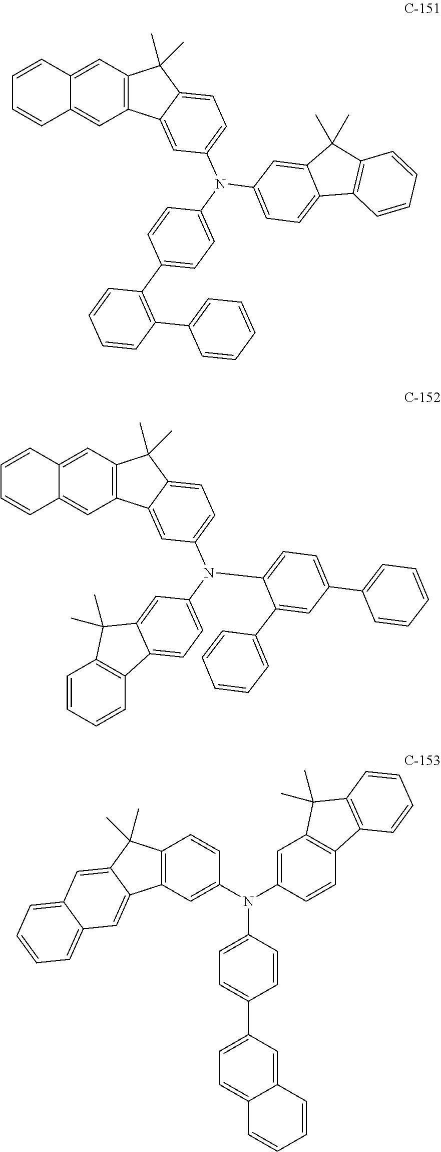 [SMILES: CC1(C)c2ccccc2-c2ccc(N(c3ccc(-c4ccccc4-c4ccccc4)cc3)c3ccc4c(c3)-c3cc5ccccc5cc3C4(C)C)cc21 ; CC1(C)c2ccccc2-c2ccc(N(c3ccc4c(c3)-c3cc5ccccc5cc3C4(C)C)c3ccc(-c4ccccc4)cc3-c3ccccc3)cc21 ; CC1(C)c2ccccc2-c2ccc(N(c3ccc(-c4ccc5ccccc5c4)cc3)c3ccc4c(c3)-c3cc5ccccc5cc3C4(C)C)cc21] 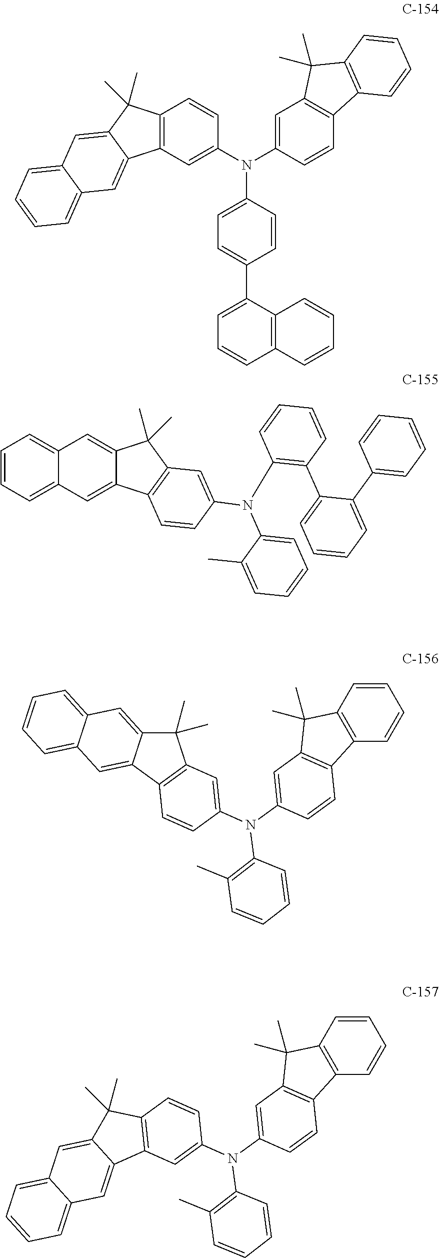 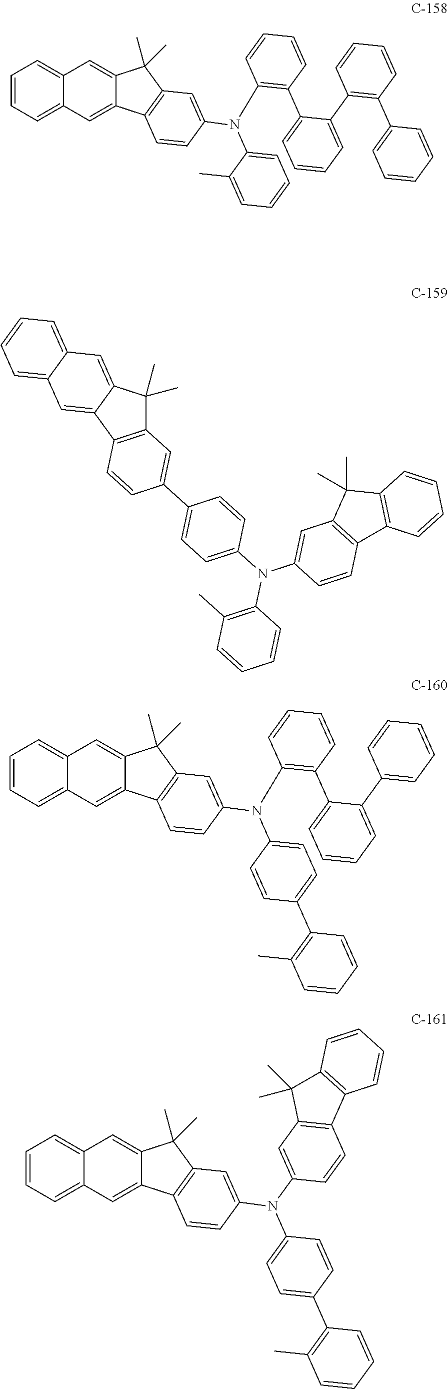 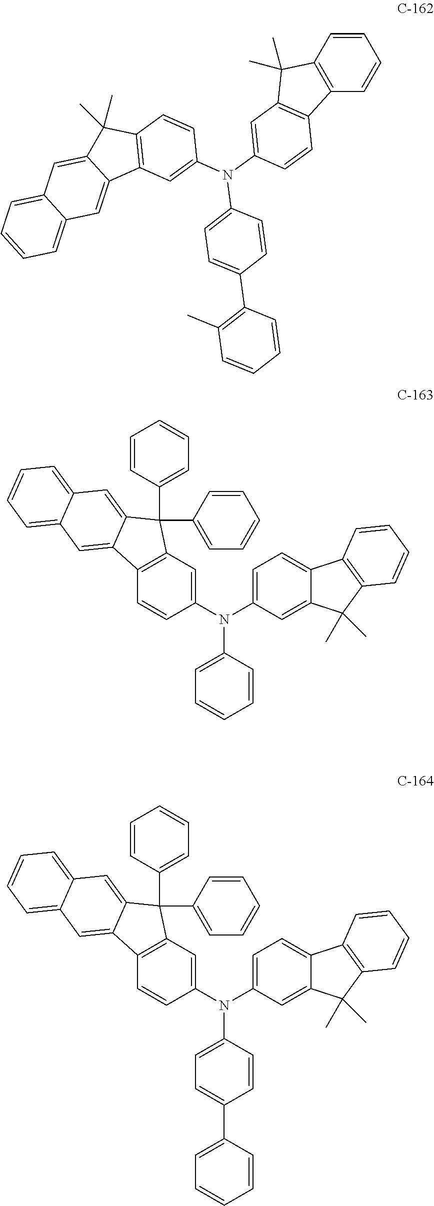 [SMILES: Cc1ccccc1-c1ccc(N(c2ccc3c(c2)-c2cc4ccccc4cc2C3(C)C)c2ccc3c(c2)C(C)(C)c2ccccc2-3)cc1 ; CC1(C)c2ccccc2-c2ccc(N(c3ccccc3)c3ccc4c(c3)C(c3ccccc3)(c3ccccc3)c3cc5ccccc5cc3-4)cc21 ; CC1(C)c2ccccc2-c2ccc(N(c3ccc(-c4ccccc4)cc3)c3ccc4c(c3)C(c3ccccc3)(c3ccccc3)c3cc5ccccc5cc3-4)cc21] 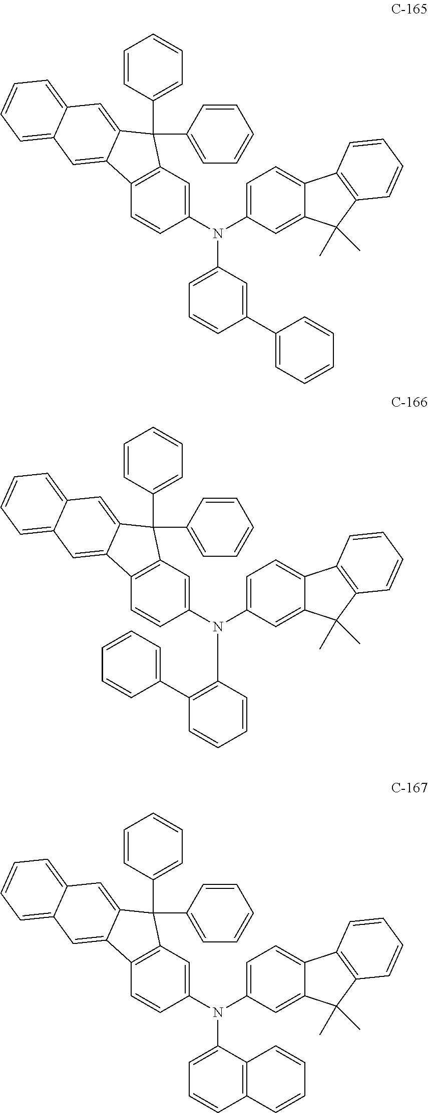 [SMILES: CC1(C)c2ccccc2-c2ccc(N(c3cccc(-c4ccccc4)c3)c3ccc4c(c3)C(c3ccccc3)(c3ccccc3)c3cc5ccccc5cc3-4)cc21 ; CC1(C)c2ccccc2-c2ccc(N(c3ccc4c(c3)C(c3ccccc3)(c3ccccc3)c3cc5ccccc5cc3-4)c3ccccc3-c3ccccc3)cc21 ; CC1(C)c2ccccc2-c2ccc(N(c3ccc4c(c3)C(c3ccccc3)(c3ccccc3)c3cc5ccccc5cc3-4)c3cccc4ccccc34)cc21] 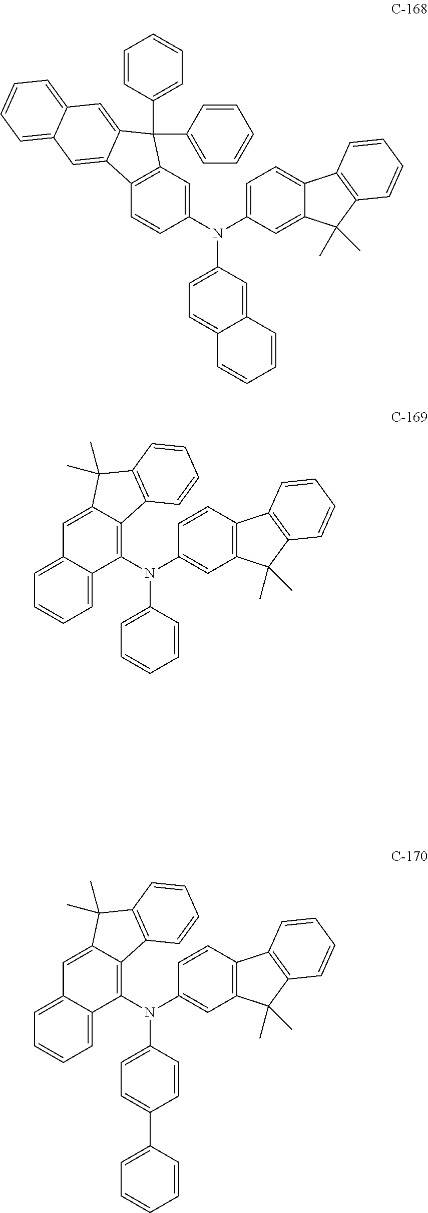 [SMILES: CC1(C)c2ccccc2-c2ccc(N(c3ccc4c(c3)C(c3ccccc3)(c3ccccc3)c3cc5ccccc5cc3-4)c3ccc4ccccc4c3)cc21 ; CC1(C)c2ccccc2-c2ccc(N(c3ccccc3)c3c4c(cc5ccccc35)C(C)(C)c3ccccc3-4)cc21 ; CC1(C)c2ccccc2-c2ccc(N(c3ccc(-c4ccccc4)cc3)c3c4c(cc5ccccc35)C(C)(C)c3ccccc3-4)cc21] 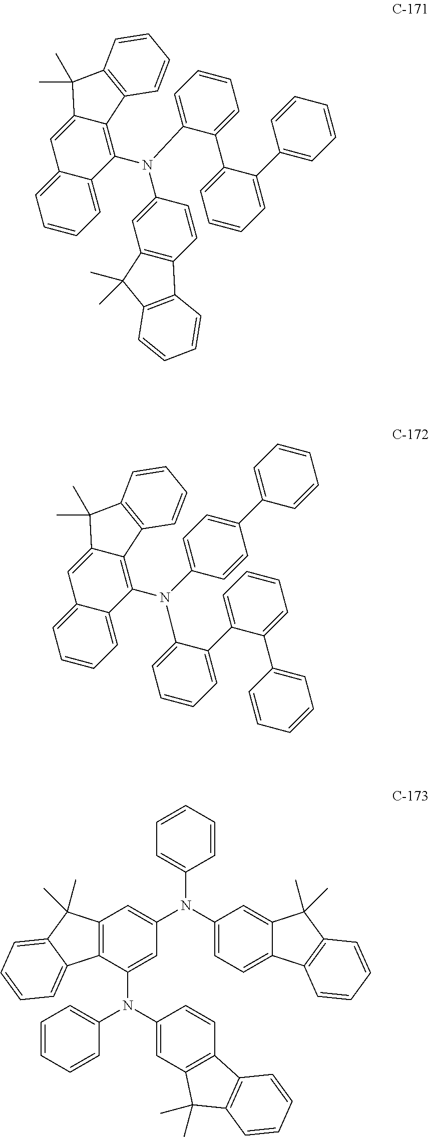 [SMILES: CC1(C)c2ccccc2-c2ccc(N(c3ccccc3-c3ccccc3-c3ccccc3)c3c4c(cc5ccccc35)C(C)(C)c3ccccc3-4)cc21 ; CC1(C)c2ccccc2-c2c1cc1ccccc1c2N(c1ccc(-c2ccccc2)cc1)c1ccccc1-c1ccccc1-c1ccccc1 ; CC1(C)c2ccccc2-c2ccc(N(c3ccccc3)c3cc(N(c4ccccc4)c4ccc5c(c4)C(C)(C)c4ccccc4-5)c4c(c3)C(C)(C)c3ccccc3-4)cc21] 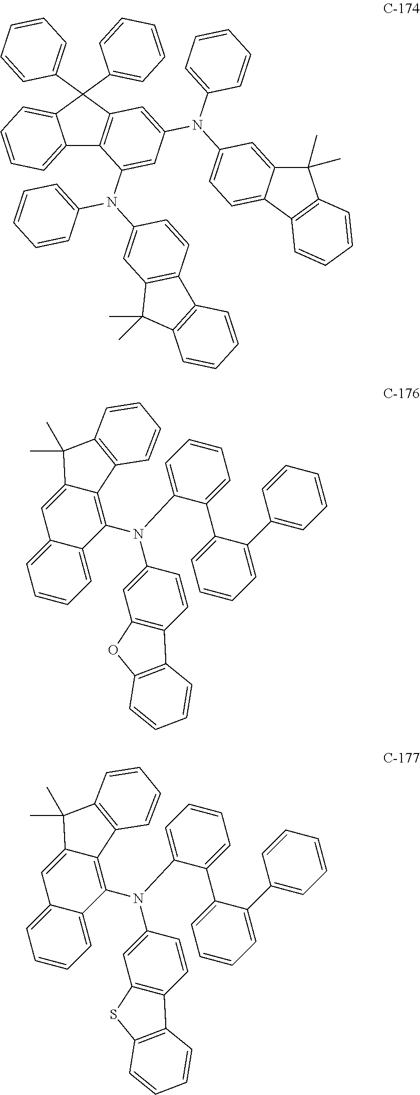 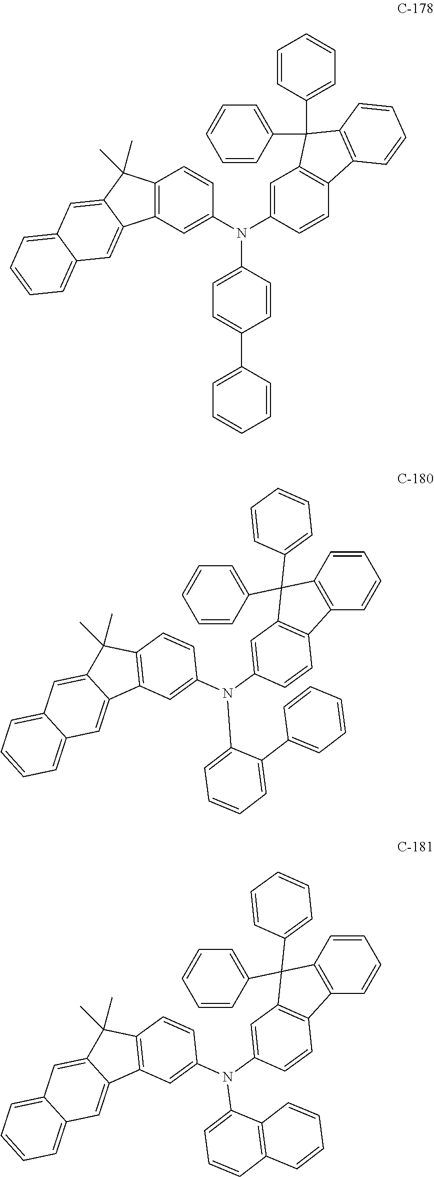 [SMILES: CC1(C)c2ccc(N(c3ccc(-c4ccccc4)cc3)c3ccc4c(c3)C(c3ccccc3)(c3ccccc3)c3ccccc3-4)cc2-c2cc3ccccc3cc21 ; CC1(C)c2ccc(N(c3ccc4c(c3)C(c3ccccc3)(c3ccccc3)c3ccccc3-4)c3ccccc3-c3ccccc3)cc2-c2cc3ccccc3cc21 ; CC1(C)c2ccc(N(c3ccc4c(c3)C(c3ccccc3)(c3ccccc3)c3ccccc3-4)c3cccc4ccccc34)cc2-c2cc3ccccc3cc21] 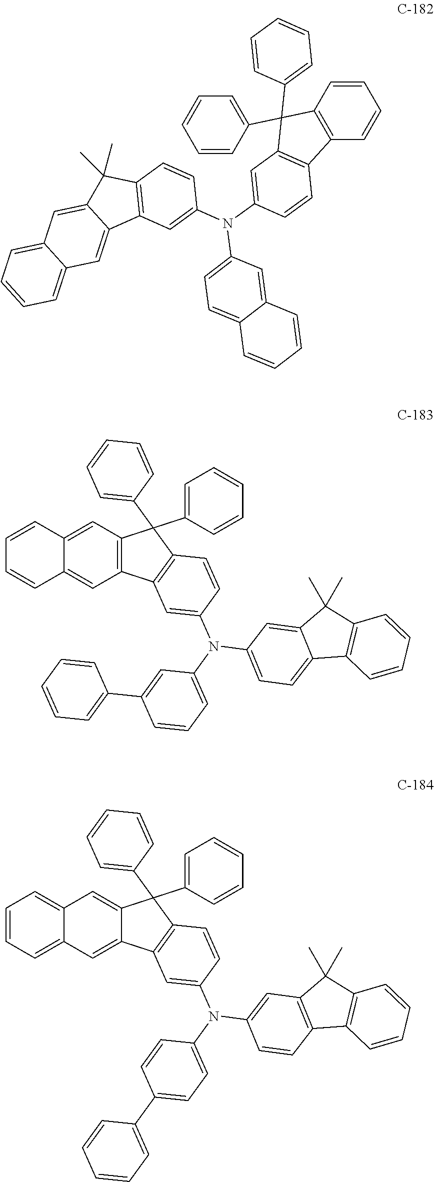 [SMILES: CC1(C)c2ccc(N(c3ccc4c(c3)C(c3ccccc3)(c3ccccc3)c3ccccc3-4)c3ccc4ccccc4c3)cc2-c2cc3ccccc3cc21 ; CC1(C)c2ccccc2-c2ccc(N(c3cccc(-c4ccccc4)c3)c3ccc4c(c3)-c3cc5ccccc5cc3C4(c3ccccc3)c3ccccc3)cc21 ; CC1(C)c2ccccc2-c2ccc(N(c3ccc(-c4ccccc4)cc3)c3ccc4c(c3)-c3cc5ccccc5cc3C4(c3ccccc3)c3ccccc3)cc21] 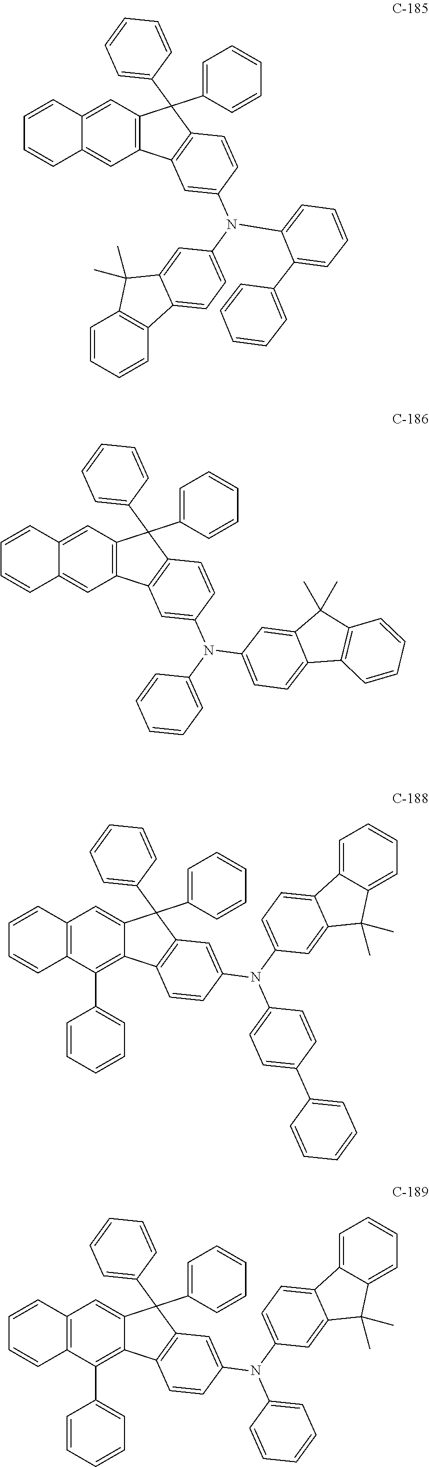 [SMILES: CC1(C)c2ccccc2-c2ccc(N(c3ccc4c(c3)-c3cc5ccccc5cc3C4(c3ccccc3)c3ccccc3)c3ccccc3-c3ccccc3)cc21 ; CC1(C)c2ccccc2-c2ccc(N(c3ccccc3)c3ccc4c(c3)-c3cc5ccccc5cc3C4(c3ccccc3)c3ccccc3)cc21 ; CC1(C)c2ccccc2-c2ccc(N(c3ccc(-c4ccccc4)cc3)c3ccc4c(c3)C(c3ccccc3)(c3ccccc3)c3cc5ccccc5c(-c5ccccc5)c3-4)cc21 ; CC1(C)c2ccccc2-c2ccc(N(c3ccccc3)c3ccc4c(c3)C(c3ccccc3)(c3ccccc3)c3cc5ccccc5c(-c5ccccc5)c3-4)cc21] 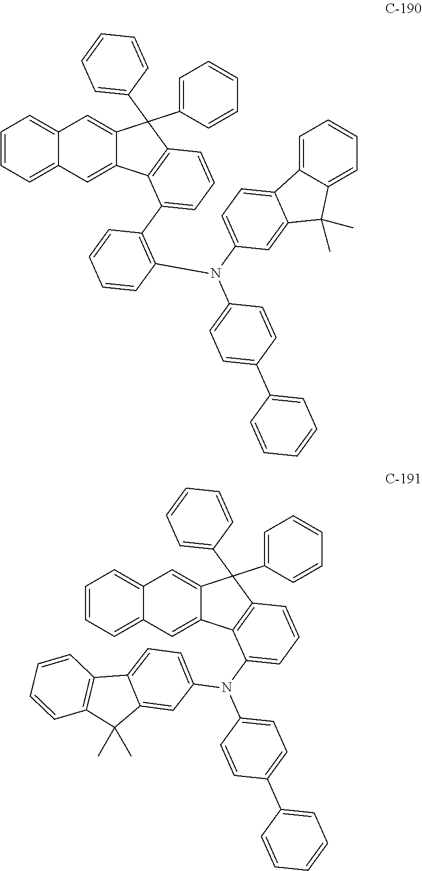 [SMILES: CC1(C)c2ccccc2-c2ccc(N(c3ccc(-c4ccccc4)cc3)c3ccccc3-c3cccc4c3-c3cc5ccccc5cc3C4(c3ccccc3)c3ccccc3)cc21 ; CC1(C)c2ccccc2-c2ccc(N(c3ccc(-c4ccccc4)cc3)c3cccc4c3-c3cc5ccccc5cc3C4(c3ccccc3)c3ccccc3)cc21] 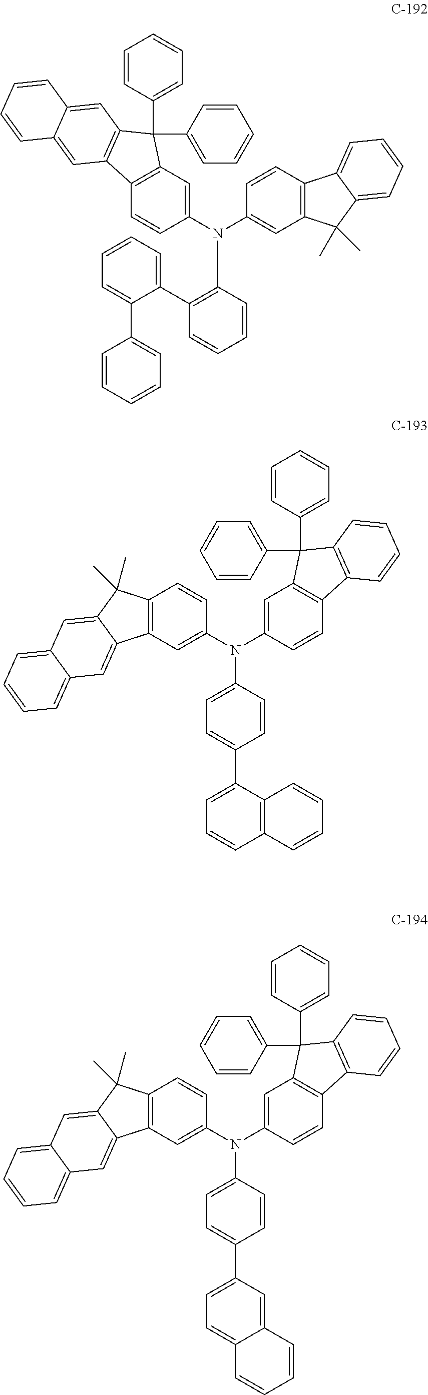 [SMILES: CC1(C)c2ccccc2-c2ccc(N(c3ccc4c(c3)C(c3ccccc3)(c3ccccc3)c3cc5ccccc5cc3-4)c3ccccc3-c3ccccc3-c3ccccc3)cc21 ; CC1(C)c2ccc(N(c3ccc(-c4cccc5ccccc45)cc3)c3ccc4c(c3)C(c3ccccc3)(c3ccccc3)c3ccccc3-4)cc2-c2cc3ccccc3cc21 ; CC1(C)c2ccc(N(c3ccc(-c4ccc5ccccc5c4)cc3)c3ccc4c(c3)C(c3ccccc3)(c3ccccc3)c3ccccc3-4)cc2-c2cc3ccccc3cc21] 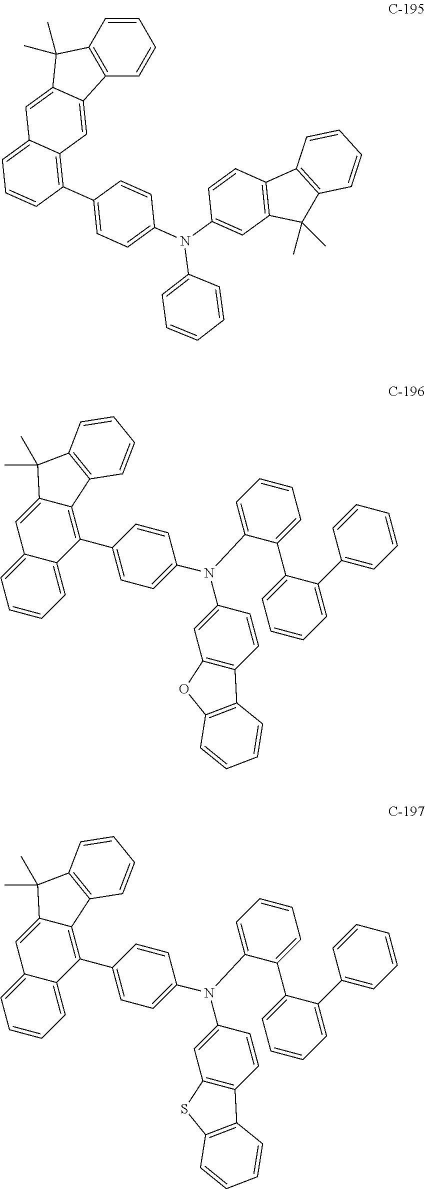 [SMILES: CC1(C)c2ccccc2-c2ccc(N(c3ccccc3)c3ccc(-c4cccc5cc6c(cc45)-c4ccccc4C6(C)C)cc3)cc21 ; CC1(C)c2ccccc2-c2c1cc1ccccc1c2-c1ccc(N(c2ccc3c(c2)oc2ccccc23)c2ccccc2-c2ccccc2-c2ccccc2)cc1 ; CC1(C)c2ccccc2-c2c1cc1ccccc1c2-c1ccc(N(c2ccc3c(c2)sc2ccccc23)c2ccccc2-c2ccccc2-c2ccccc2)cc1] 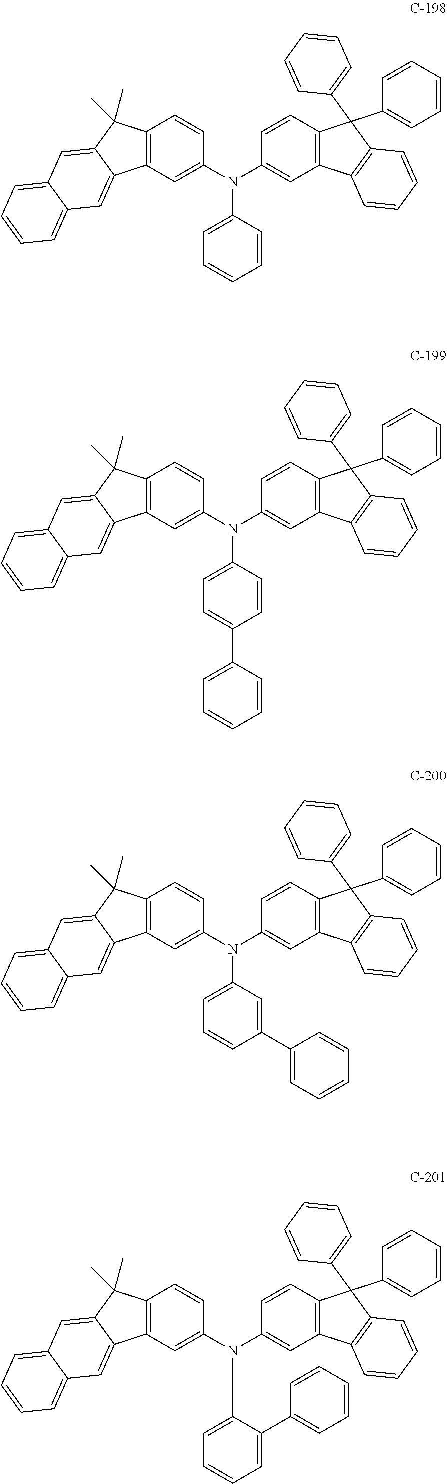 [SMILES: CC1(C)c2ccc(N(c3ccccc3)c3ccc4c(c3)-c3ccccc3C4(c3ccccc3)c3ccccc3)cc2-c2cc3ccccc3cc21 ; CC1(C)c2ccc(N(c3ccc(-c4ccccc4)cc3)c3ccc4c(c3)-c3ccccc3C4(c3ccccc3)c3ccccc3)cc2-c2cc3ccccc3cc21 ; CC1(C)c2ccc(N(c3cccc(-c4ccccc4)c3)c3ccc4c(c3)-c3ccccc3C4(c3ccccc3)c3ccccc3)cc2-c2cc3ccccc3cc21 ; CC1(C)c2ccc(N(c3ccc4c(c3)-c3ccccc3C4(c3ccccc3)c3ccccc3)c3ccccc3-c3ccccc3)cc2-c2cc3ccccc3cc21] 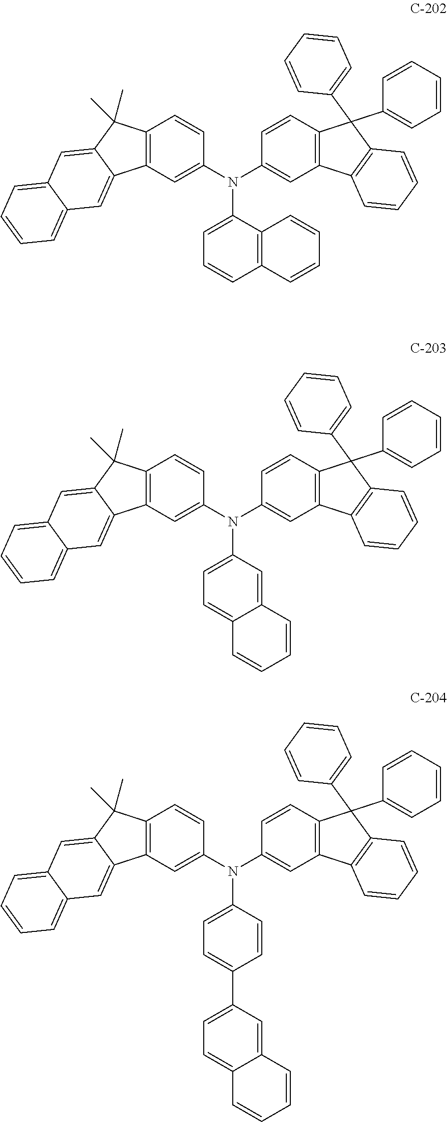 [SMILES: CC1(C)c2ccc(N(c3ccc4c(c3)-c3ccccc3C4(c3ccccc3)c3ccccc3)c3cccc4ccccc34)cc2-c2cc3ccccc3cc21 ; CC1(C)c2ccc(N(c3ccc4c(c3)-c3ccccc3C4(c3ccccc3)c3ccccc3)c3ccc4ccccc4c3)cc2-c2cc3ccccc3cc21 ; CC1(C)c2ccc(N(c3ccc(-c4ccc5ccccc5c4)cc3)c3ccc4c(c3)-c3ccccc3C4(c3ccccc3)c3ccccc3)cc2-c2cc3ccccc3cc21] 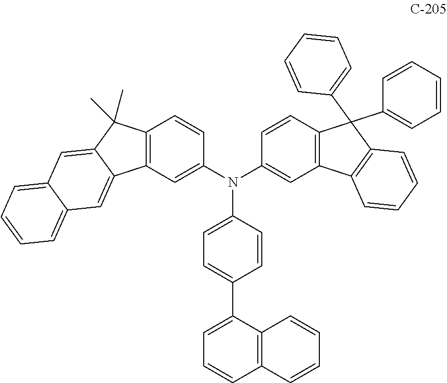 [SMILES: CC1(C)c2ccc(N(c3ccc(-c4cccc5ccccc45)cc3)c3ccc4c(c3)-c3ccccc3C4(c3ccccc3)c3ccccc3)cc2-c2cc3ccccc3cc21] 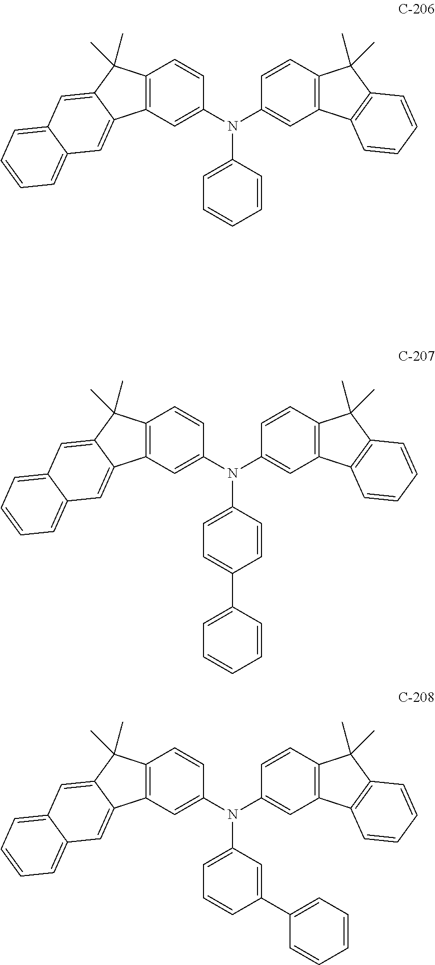 [SMILES: CC1(C)c2ccccc2-c2cc(N(c3ccccc3)c3ccc4c(c3)-c3cc5ccccc5cc3C4(C)C)ccc21 ; CC1(C)c2ccccc2-c2cc(N(c3ccc(-c4ccccc4)cc3)c3ccc4c(c3)-c3cc5ccccc5cc3C4(C)C)ccc21 ; CC1(C)c2ccccc2-c2cc(N(c3cccc(-c4ccccc4)c3)c3ccc4c(c3)-c3cc5ccccc5cc3C4(C)C)ccc21] 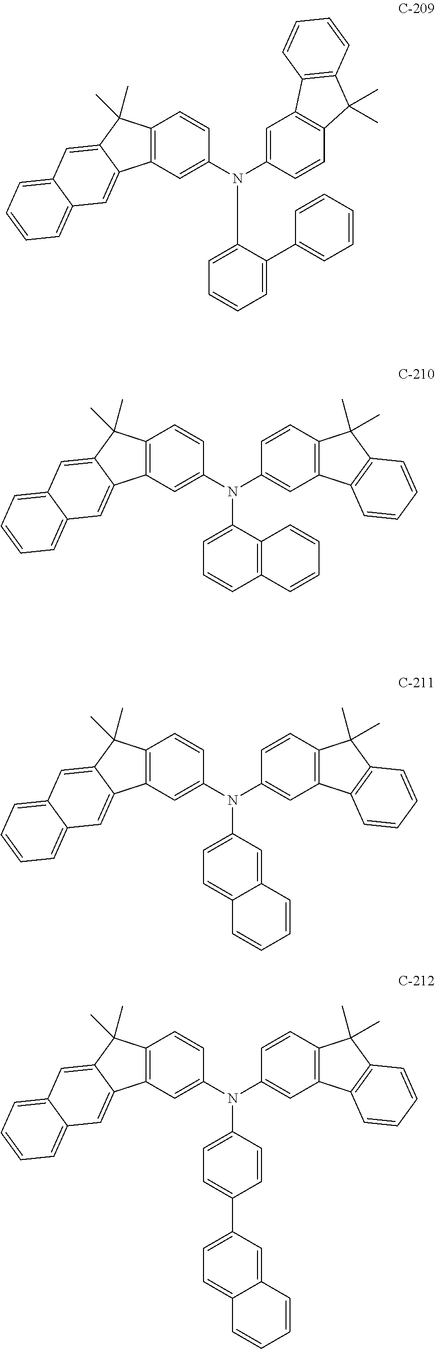 [SMILES: CC1(C)c2ccccc2-c2cc(N(c3ccc4c(c3)-c3cc5ccccc5cc3C4(C)C)c3ccccc3-c3ccccc3)ccc21 ; CC1(C)c2ccccc2-c2cc(N(c3ccc4c(c3)-c3cc5ccccc5cc3C4(C)C)c3cccc4ccccc34)ccc21 ; CC1(C)c2ccccc2-c2cc(N(c3ccc4c(c3)-c3cc5ccccc5cc3C4(C)C)c3ccc4ccccc4c3)ccc21 ; CC1(C)c2ccccc2-c2cc(N(c3ccc(-c4ccc5ccccc5c4)cc3)c3ccc4c(c3)-c3cc5ccccc5cc3C4(C)C)ccc21] 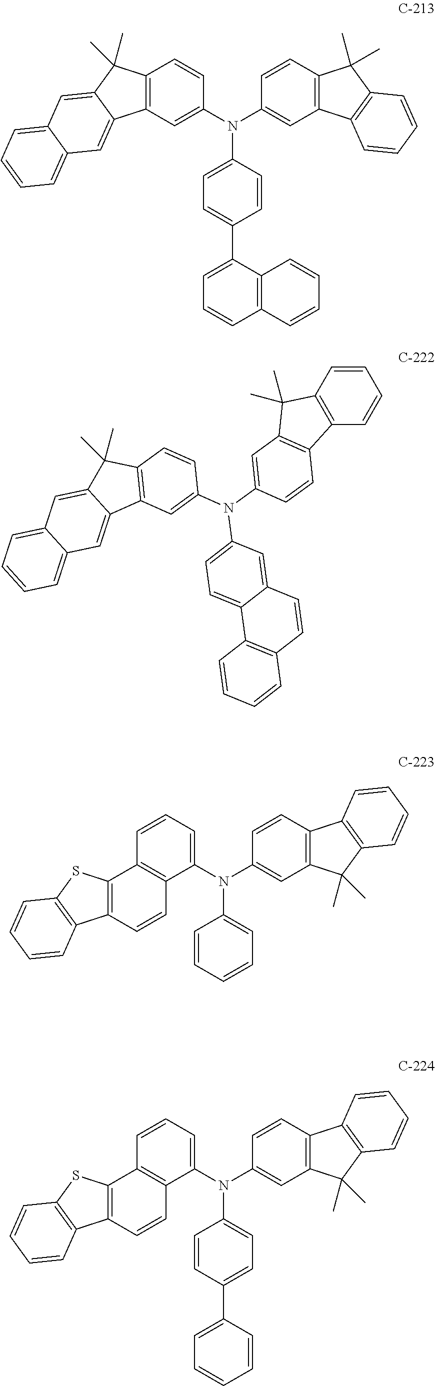 [SMILES: CC1(C)c2ccccc2-c2cc(N(c3ccc(-c4cccc5ccccc45)cc3)c3ccc4c(c3)-c3cc5ccccc5cc3C4(C)C)ccc21 ; CC1(C)c2ccccc2-c2ccc(N(c3ccc4c(c3)-c3cc5ccccc5cc3C4(C)C)c3ccc4c(ccc5ccccc54)c3)cc21 ; CC1(C)c2ccccc2-c2ccc(N(c3ccccc3)c3cccc4c3ccc3c5ccccc5sc43)cc21 ; CC1(C)c2ccccc2-c2ccc(N(c3ccc(-c4ccccc4)cc3)c3cccc4c3ccc3c5ccccc5sc43)cc21] 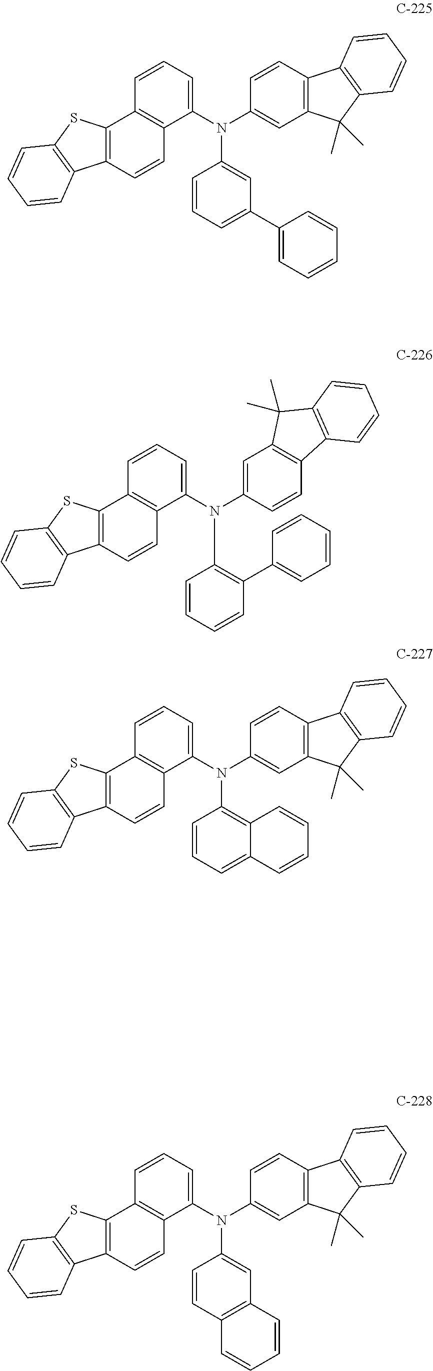 [SMILES: CC1(C)c2ccccc2-c2ccc(N(c3cccc(-c4ccccc4)c3)c3cccc4c3ccc3c5ccccc5sc43)cc21 ; CC1(C)c2ccccc2-c2ccc(N(c3ccccc3-c3ccccc3)c3cccc4c3ccc3c5ccccc5sc43)cc21 ; CC1(C)c2ccccc2-c2ccc(N(c3cccc4ccccc34)c3cccc4c3ccc3c5ccccc5sc43)cc21 ; CC1(C)c2ccccc2-c2ccc(N(c3ccc4ccccc4c3)c3cccc4c3ccc3c5ccccc5sc43)cc21] 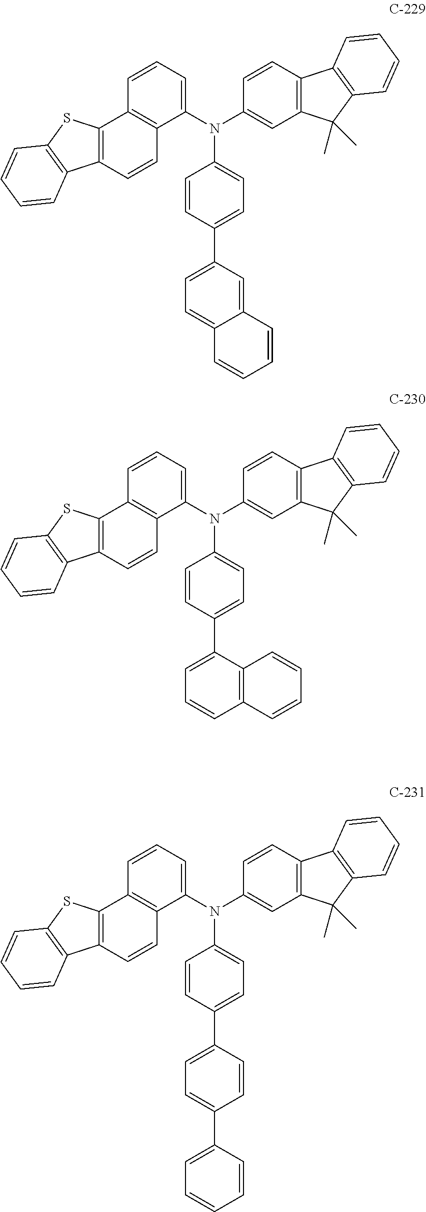 [SMILES: CC1(C)c2ccccc2-c2ccc(N(c3ccc(-c4ccc5ccccc5c4)cc3)c3cccc4c3ccc3c5ccccc5sc43)cc21 ; CC1(C)c2ccccc2-c2ccc(N(c3ccc(-c4cccc5ccccc45)cc3)c3cccc4c3ccc3c5ccccc5sc43)cc21 ; CC1(C)c2ccccc2-c2ccc(N(c3ccc(-c4ccc(-c5ccccc5)cc4)cc3)c3cccc4c3ccc3c5ccccc5sc43)cc21] 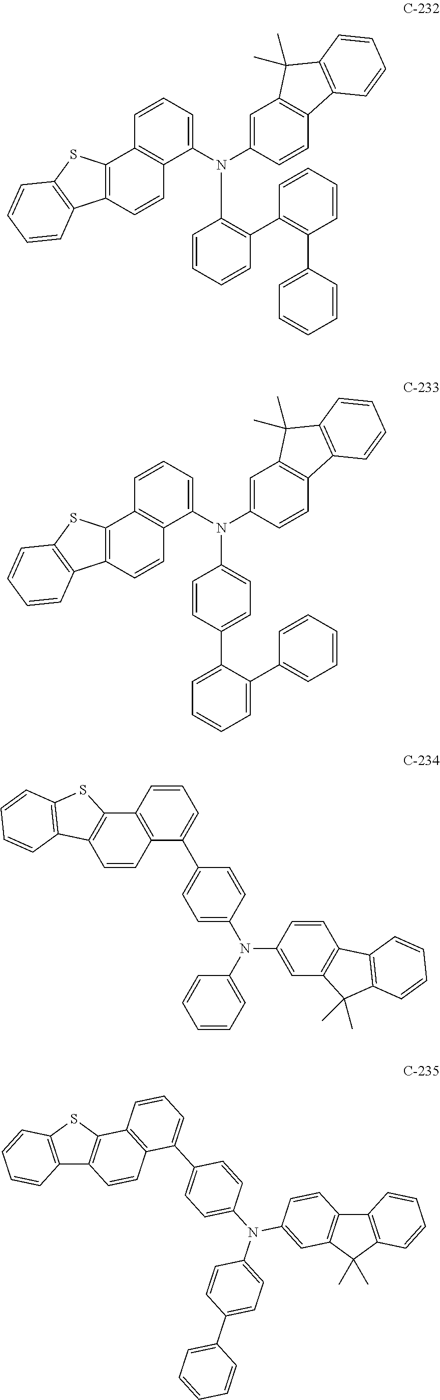 [SMILES: CC1(C)c2ccccc2-c2ccc(N(c3ccccc3-c3ccccc3-c3ccccc3)c3cccc4c3ccc3c5ccccc5sc43)cc21 ; CC1(C)c2ccccc2-c2ccc(N(c3ccc(-c4ccccc4-c4ccccc4)cc3)c3cccc4c3ccc3c5ccccc5sc43)cc21 ; CC1(C)c2ccccc2-c2ccc(N(c3ccccc3)c3ccc(-c4cccc5c4ccc4c6ccccc6sc54)cc3)cc21 ; CC1(C)c2ccccc2-c2ccc(N(c3ccc(-c4ccccc4)cc3)c3ccc(-c4cccc5c4ccc4c6ccccc6sc54)cc3)cc21] 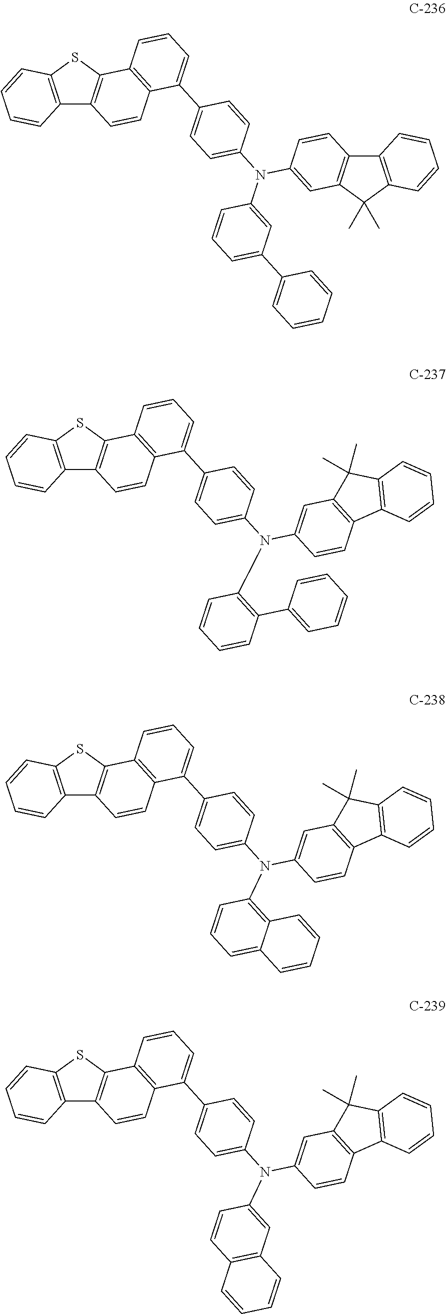 [SMILES: CC1(C)c2ccccc2-c2ccc(N(c3ccc(-c4cccc5c4ccc4c6ccccc6sc54)cc3)c3cccc(-c4ccccc4)c3)cc21 ; CC1(C)c2ccccc2-c2ccc(N(c3ccc(-c4cccc5c4ccc4c6ccccc6sc54)cc3)c3ccccc3-c3ccccc3)cc21 ; CC1(C)c2ccccc2-c2ccc(N(c3ccc(-c4cccc5c4ccc4c6ccccc6sc54)cc3)c3cccc4ccccc34)cc21 ; CC1(C)c2ccccc2-c2ccc(N(c3ccc(-c4cccc5c4ccc4c6ccccc6sc54)cc3)c3ccc4ccccc4c3)cc21] 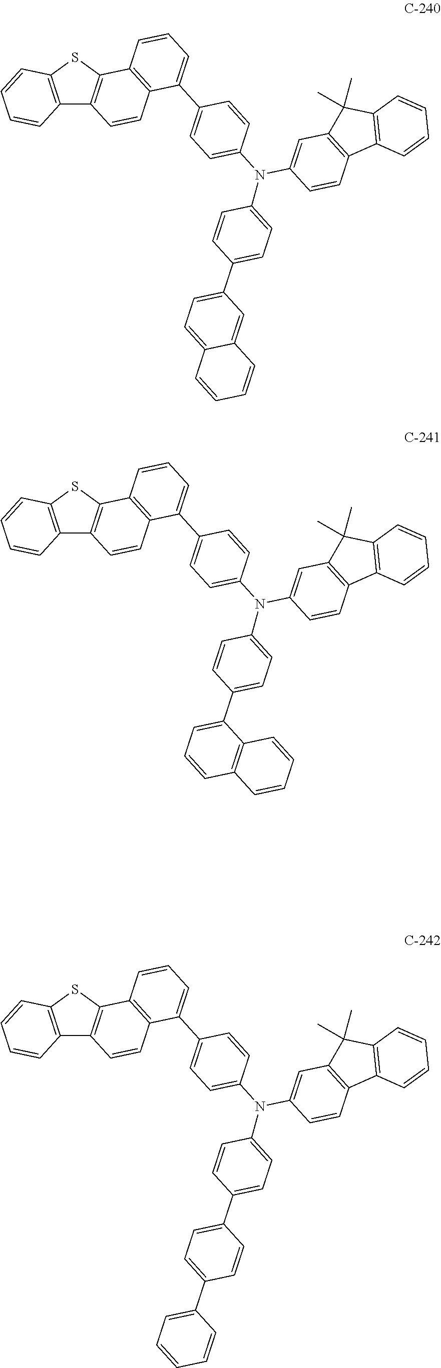 [SMILES: CC1(C)c2ccccc2-c2ccc(N(c3ccc(-c4ccc5ccccc5c4)cc3)c3ccc(-c4cccc5c4ccc4c6ccccc6sc54)cc3)cc21 ; CC1(C)c2ccccc2-c2ccc(N(c3ccc(-c4cccc5ccccc45)cc3)c3ccc(-c4cccc5c4ccc4c6ccccc6sc54)cc3)cc21 ; CC1(C)c2ccccc2-c2ccc(N(c3ccc(-c4ccc(-c5ccccc5)cc4)cc3)c3ccc(-c4cccc5c4ccc4c6ccccc6sc54)cc3)cc21] 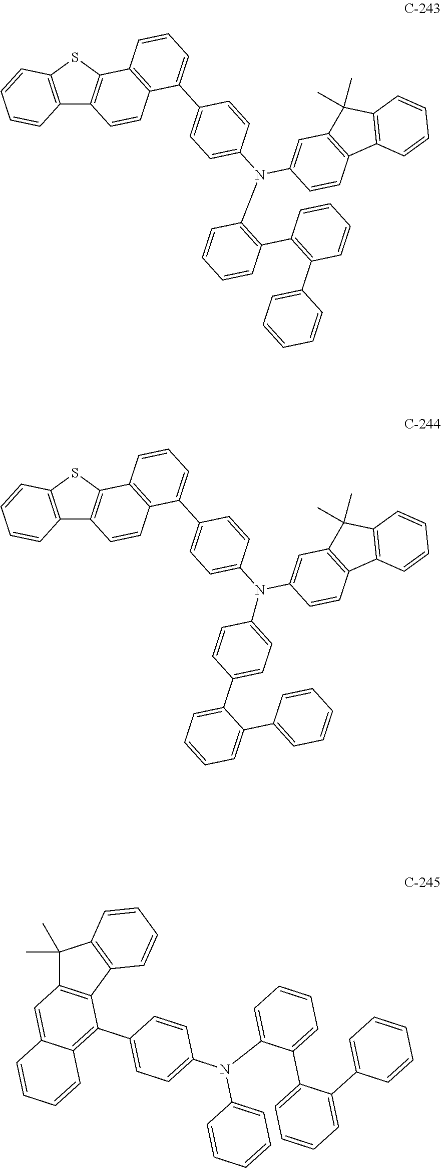 [SMILES: CC1(C)c2ccccc2-c2ccc(N(c3ccc(-c4cccc5c4ccc4c6ccccc6sc54)cc3)c3ccccc3-c3ccccc3-c3ccccc3)cc21 ; CC1(C)c2ccccc2-c2ccc(N(c3ccc(-c4ccccc4-c4ccccc4)cc3)c3ccc(-c4cccc5c4ccc4c6ccccc6sc54)cc3)cc21 ; CC1(C)c2ccccc2-c2c1cc1ccccc1c2-c1ccc(N(c2ccccc2)c2ccccc2-c2ccccc2-c2ccccc2)cc1] 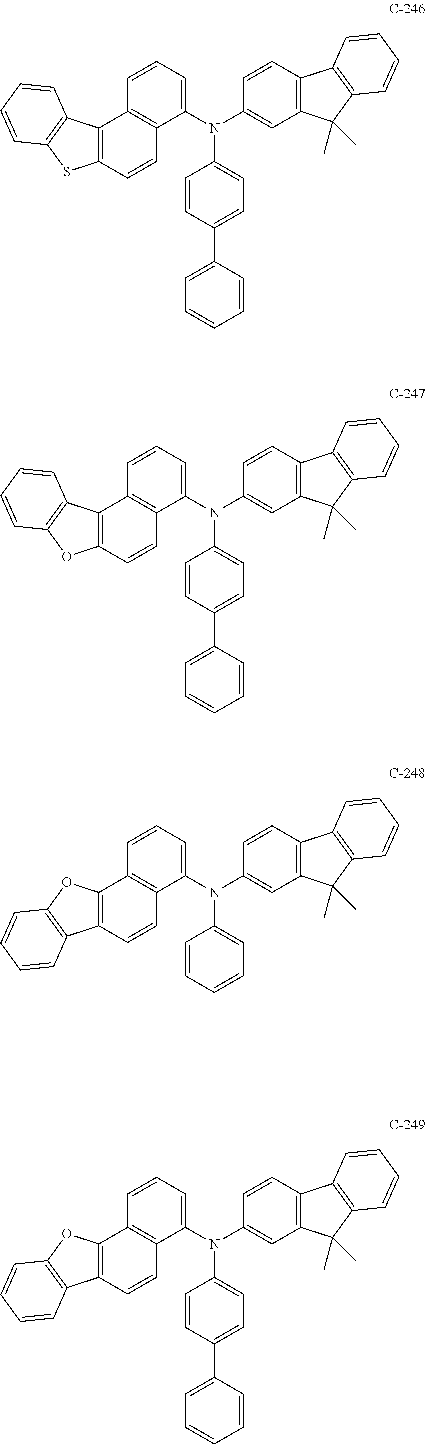 [SMILES: CC1(C)c2ccccc2-c2ccc(N(c3ccc(-c4ccccc4)cc3)c3cccc4c3ccc3sc5ccccc5c34)cc21 ; CC1(C)c2ccccc2-c2ccc(N(c3ccc(-c4ccccc4)cc3)c3cccc4c3ccc3oc5ccccc5c34)cc21 ; CC1(C)c2ccccc2-c2ccc(N(c3ccccc3)c3cccc4c3ccc3c5ccccc5oc43)cc21 ; CC1(C)c2ccccc2-c2ccc(N(c3ccc(-c4ccccc4)cc3)c3cccc4c3ccc3c5ccccc5oc43)cc21] 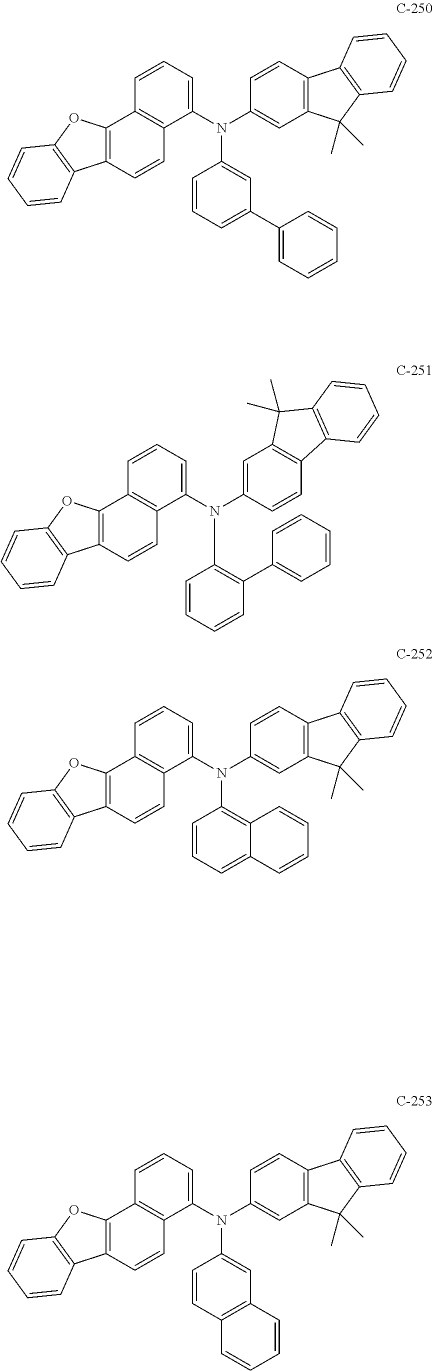 [SMILES: CC1(C)c2ccccc2-c2ccc(N(c3cccc(-c4ccccc4)c3)c3cccc4c3ccc3c5ccccc5oc43)cc21 ; CC1(C)c2ccccc2-c2ccc(N(c3ccccc3-c3ccccc3)c3cccc4c3ccc3c5ccccc5oc43)cc21 ; CC1(C)c2ccccc2-c2ccc(N(c3cccc4ccccc34)c3cccc4c3ccc3c5ccccc5oc43)cc21 ; CC1(C)c2ccccc2-c2ccc(N(c3ccc4ccccc4c3)c3cccc4c3ccc3c5ccccc5oc43)cc21] 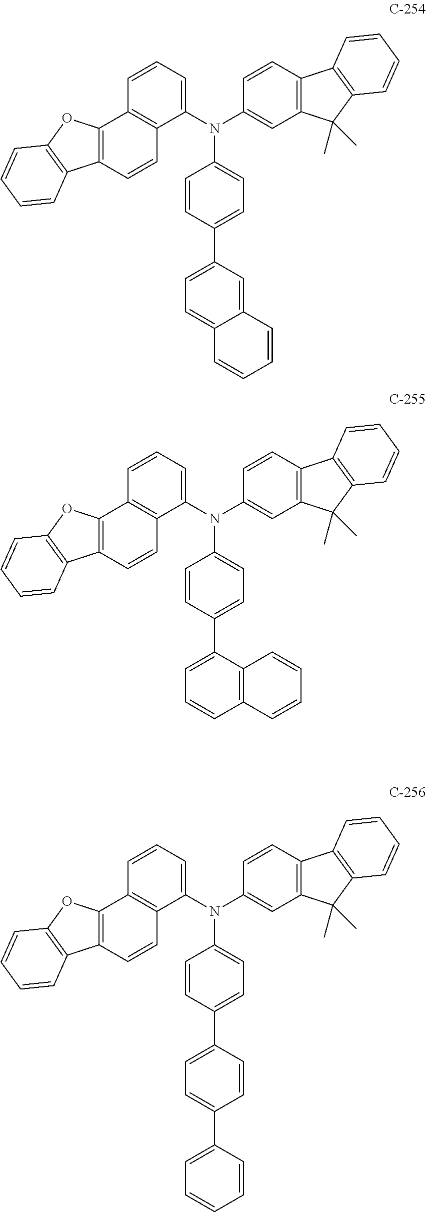 [SMILES: CC1(C)c2ccccc2-c2ccc(N(c3ccc(-c4ccc5ccccc5c4)cc3)c3cccc4c3ccc3c5ccccc5oc43)cc21 ; CC1(C)c2ccccc2-c2ccc(N(c3ccc(-c4cccc5ccccc45)cc3)c3cccc4c3ccc3c5ccccc5oc43)cc21 ; CC1(C)c2ccccc2-c2ccc(N(c3ccc(-c4ccc(-c5ccccc5)cc4)cc3)c3cccc4c3ccc3c5ccccc5oc43)cc21] 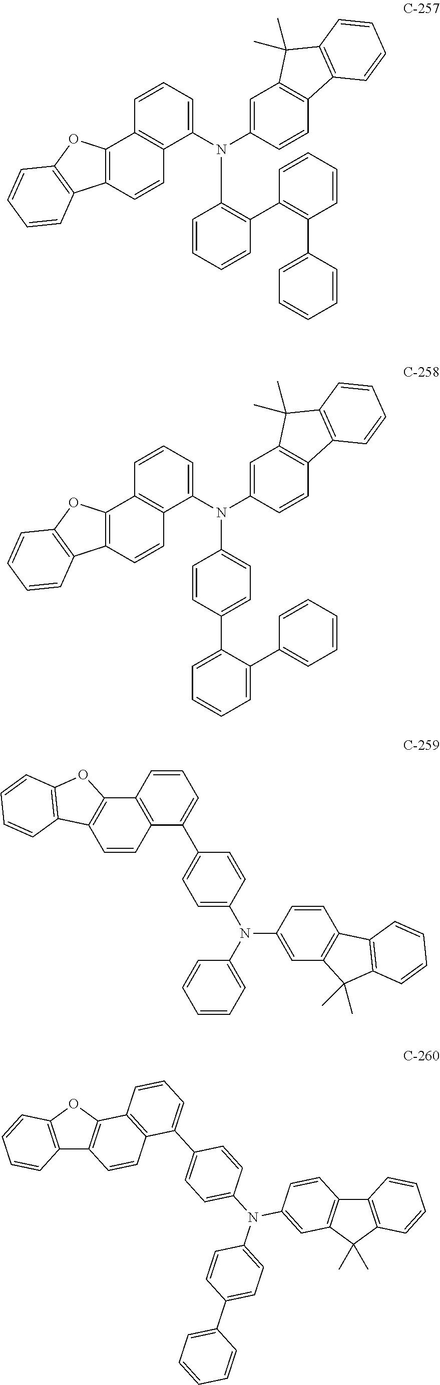 [SMILES: CC1(C)c2ccccc2-c2ccc(N(c3ccccc3-c3ccccc3-c3ccccc3)c3cccc4c3ccc3c5ccccc5oc43)cc21 ; CC1(C)c2ccccc2-c2ccc(N(c3ccc(-c4ccccc4-c4ccccc4)cc3)c3cccc4c3ccc3c5ccccc5oc43)cc21 ; CC1(C)c2ccccc2-c2ccc(N(c3ccccc3)c3ccc(-c4cccc5c4ccc4c6ccccc6oc54)cc3)cc21 ; CC1(C)c2ccccc2-c2ccc(N(c3ccc(-c4ccccc4)cc3)c3ccc(-c4cccc5c4ccc4c6ccccc6oc54)cc3)cc21] 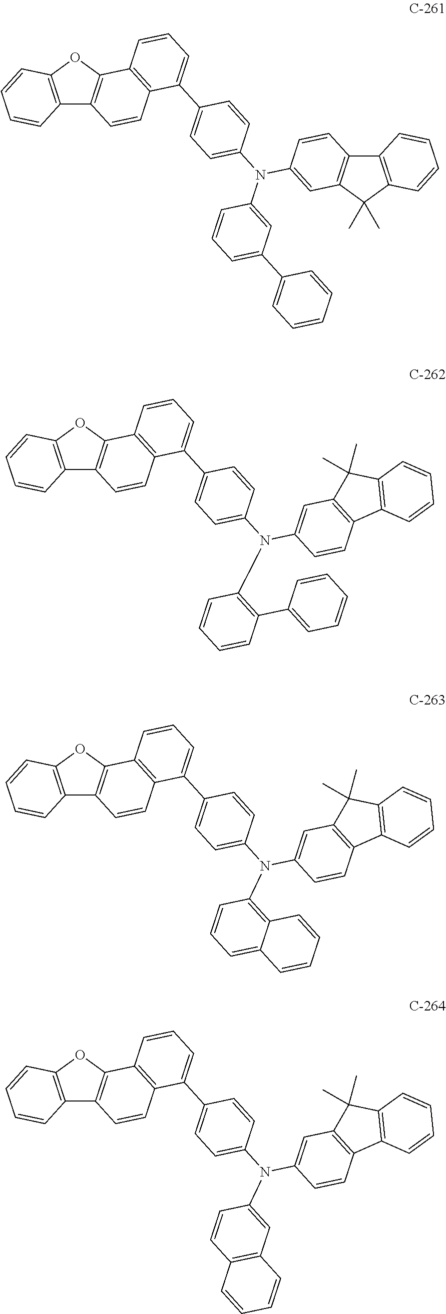 [SMILES: CC1(C)c2ccccc2-c2ccc(N(c3ccc(-c4cccc5c4ccc4c6ccccc6oc54)cc3)c3cccc(-c4ccccc4)c3)cc21 ; CC1(C)c2ccccc2-c2ccc(N(c3ccc(-c4cccc5c4ccc4c6ccccc6oc54)cc3)c3ccccc3-c3ccccc3)cc21 ; CC1(C)c2ccccc2-c2ccc(N(c3ccc(-c4cccc5c4ccc4c6ccccc6oc54)cc3)c3cccc4ccccc34)cc21 ; CC1(C)c2ccccc2-c2ccc(N(c3ccc(-c4cccc5c4ccc4c6ccccc6oc54)cc3)c3ccc4ccccc4c3)cc21] 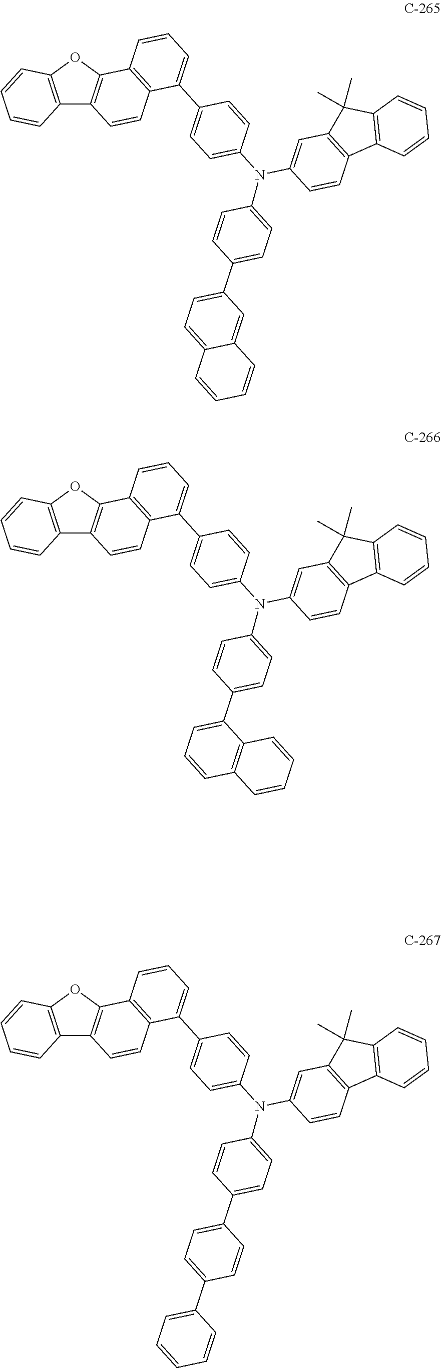 [SMILES: CC1(C)c2ccccc2-c2ccc(N(c3ccc(-c4ccc5ccccc5c4)cc3)c3ccc(-c4cccc5c4ccc4c6ccccc6oc54)cc3)cc21 ; CC1(C)c2ccccc2-c2ccc(N(c3ccc(-c4cccc5ccccc45)cc3)c3ccc(-c4cccc5c4ccc4c6ccccc6oc54)cc3)cc21 ; CC1(C)c2ccccc2-c2ccc(N(c3ccc(-c4ccc(-c5ccccc5)cc4)cc3)c3ccc(-c4cccc5c4ccc4c6ccccc6oc54)cc3)cc21] 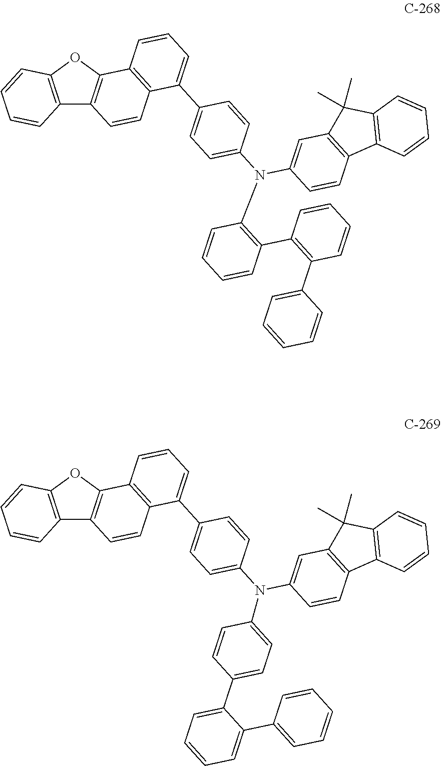 [SMILES: CC1(C)c2ccccc2-c2ccc(N(c3ccc(-c4cccc5c4ccc4c6ccccc6oc54)cc3)c3ccccc3-c3ccccc3-c3ccccc3)cc21 ; CC1(C)c2ccccc2-c2ccc(N(c3ccc(-c4ccccc4-c4ccccc4)cc3)c3ccc(-c4cccc5c4ccc4c6ccccc6oc54)cc3)cc21] 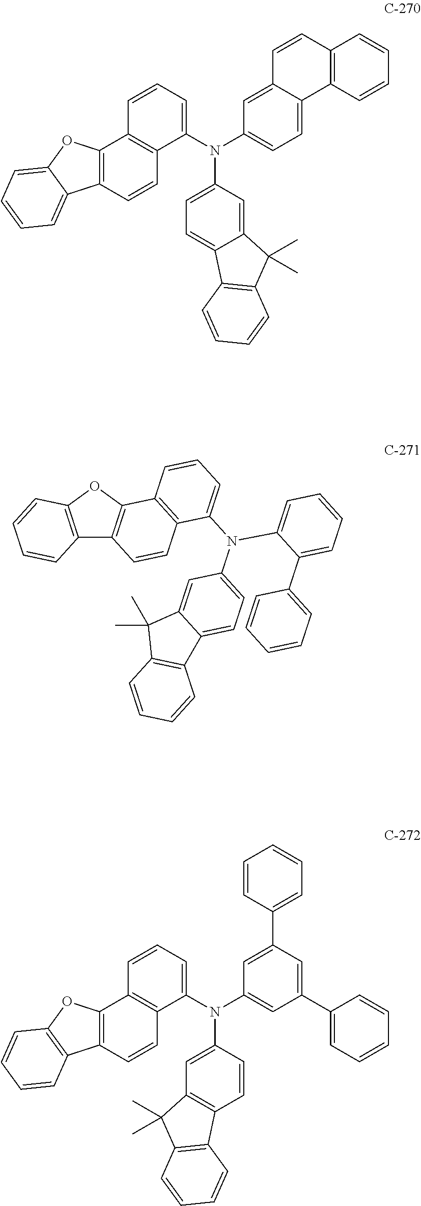 [SMILES: CC1(C)c2ccccc2-c2ccc(N(c3ccc4c(ccc5ccccc54)c3)c3cccc4c3ccc3c5ccccc5oc43)cc21 ; CC1(C)c2ccccc2-c2ccc(N(c3ccccc3-c3ccccc3)c3cccc4c3ccc3c5ccccc5oc43)cc21 ; CC1(C)c2ccccc2-c2ccc(N(c3cc(-c4ccccc4)cc(-c4ccccc4)c3)c3cccc4c3ccc3c5ccccc5oc43)cc21] 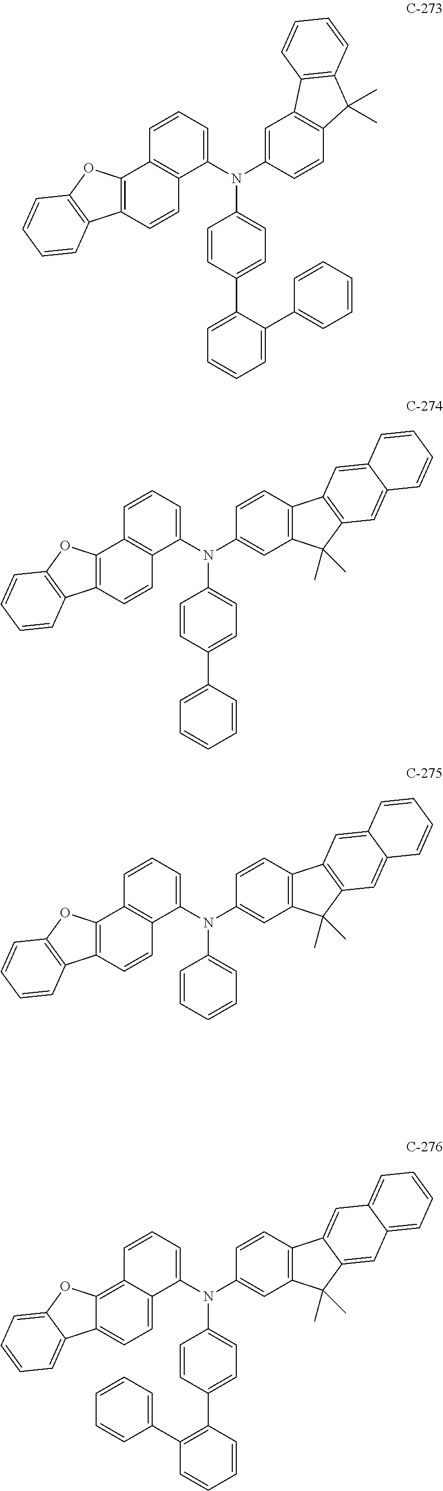 [SMILES: CC1(C)c2ccccc2-c2cc(N(c3ccc(-c4ccccc4-c4ccccc4)cc3)c3cccc4c3ccc3c5ccccc5oc43)ccc21 ; CC1(C)c2cc(N(c3ccc(-c4ccccc4)cc3)c3cccc4c3ccc3c5ccccc5oc43)ccc2-c2cc3ccccc3cc21 ; CC1(C)c2cc(N(c3ccccc3)c3cccc4c3ccc3c5ccccc5oc43)ccc2-c2cc3ccccc3cc21 ; CC1(C)c2cc(N(c3ccc(-c4ccccc4-c4ccccc4)cc3)c3cccc4c3ccc3c5ccccc5oc43)ccc2-c2cc3ccccc3cc21] 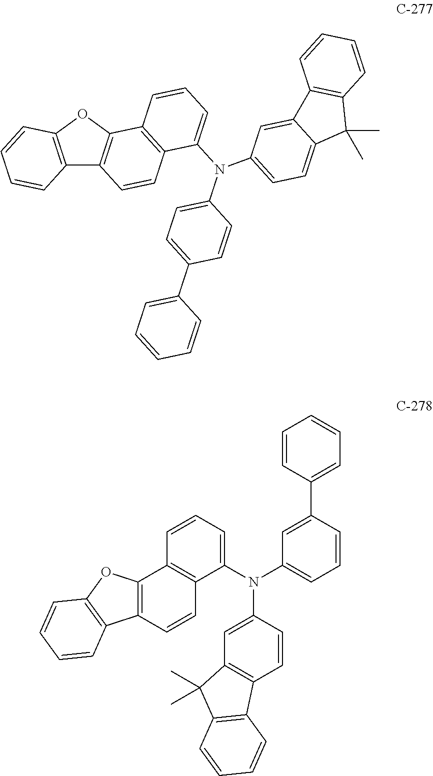 [SMILES: CC1(C)c2ccccc2-c2cc(N(c3ccc(-c4ccccc4)cc3)c3cccc4c3ccc3c5ccccc5oc43)ccc21 ; CC1(C)c2ccccc2-c2ccc(N(c3cccc(-c4ccccc4)c3)c3cccc4c3ccc3c5ccccc5oc43)cc21] 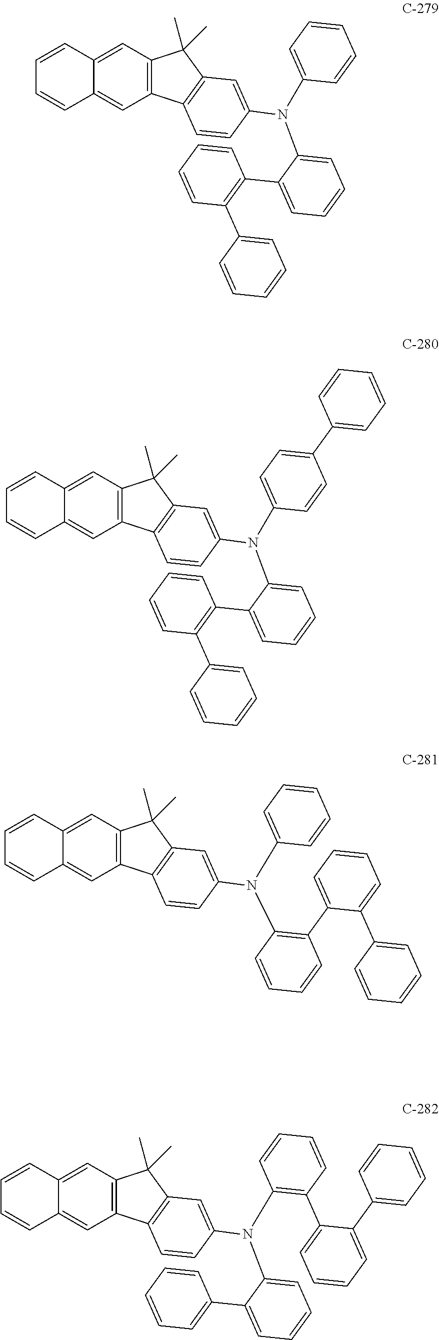 [SMILES: CC1(C)c2cc(N(c3ccccc3)c3ccccc3-c3ccccc3-c3ccccc3)ccc2-c2cc3ccccc3cc21 ; CC1(C)c2cc(N(c3ccc(-c4ccccc4)cc3)c3ccccc3-c3ccccc3-c3ccccc3)ccc2-c2cc3ccccc3cc21 ; CC1(C)c2cc(N(c3ccccc3)c3ccccc3-c3ccccc3-c3ccccc3)ccc2-c2cc3ccccc3cc21 ; CC1(C)c2cc(N(c3ccccc3-c3ccccc3)c3ccccc3-c3ccccc3-c3ccccc3)ccc2-c2cc3ccccc3cc21] 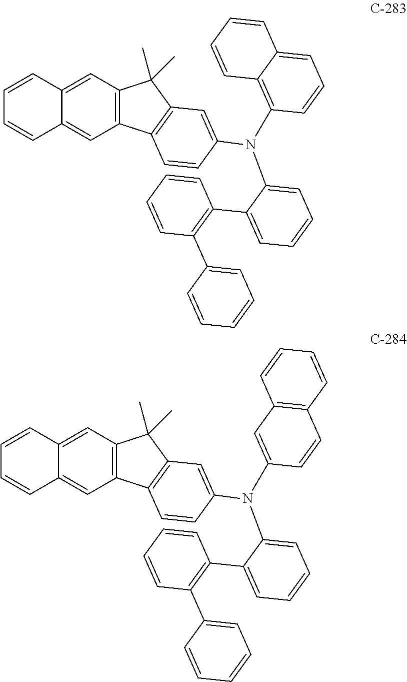 [SMILES: CC1(C)c2cc(N(c3ccccc3-c3ccccc3-c3ccccc3)c3cccc4ccccc34)ccc2-c2cc3ccccc3cc21 ; CC1(C)c2cc(N(c3ccc4ccccc4c3)c3ccccc3-c3ccccc3-c3ccccc3)ccc2-c2cc3ccccc3cc21] 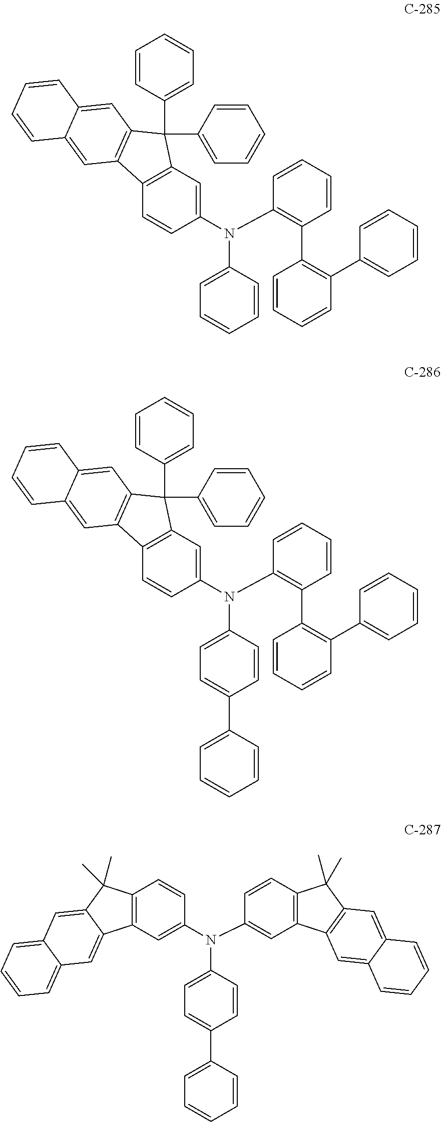 [SMILES: c1ccc(-c2ccccc2-c2ccccc2N(c2ccccc2)c2ccc3c(c2)C(c2ccccc2)(c2ccccc2)c2cc4ccccc4cc2-3)cc1 ; c1ccc(-c2ccc(N(c3ccc4c(c3)C(c3ccccc3)(c3ccccc3)c3cc5ccccc5cc3-4)c3ccccc3-c3ccccc3-c3ccccc3)cc2)cc1 ; CC1(C)c2ccc(N(c3ccc(-c4ccccc4)cc3)c3ccc4c(c3)-c3cc5ccccc5cc3C4(C)C)cc2-c2cc3ccccc3cc21] 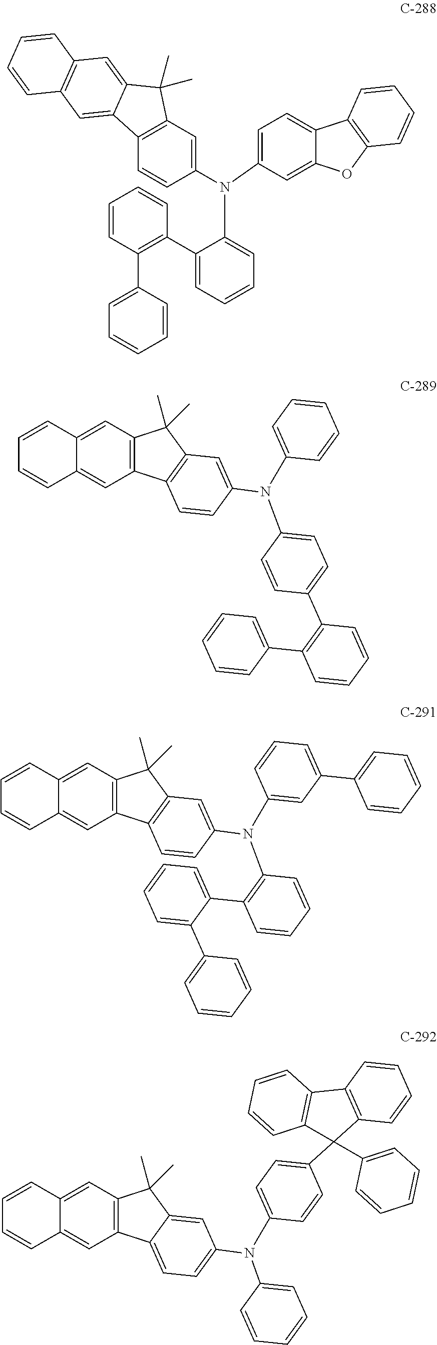 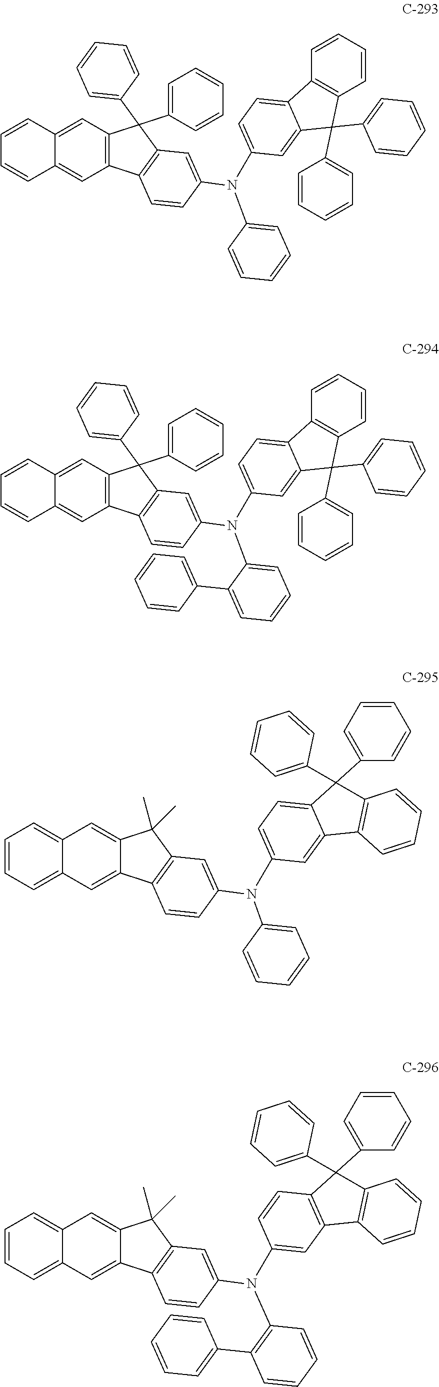 [SMILES: c1ccc(N(c2ccc3c(c2)C(c2ccccc2)(c2ccccc2)c2ccccc2-3)c2ccc3c(c2)C(c2ccccc2)(c2ccccc2)c2cc4ccccc4cc2-3)cc1 ; c1ccc(-c2ccccc2N(c2ccc3c(c2)C(c2ccccc2)(c2ccccc2)c2ccccc2-3)c2ccc3c(c2)C(c2ccccc2)(c2ccccc2)c2cc4ccccc4cc2-3)cc1 ; CC1(C)c2cc(N(c3ccccc3)c3ccc4c(c3)-c3ccccc3C4(c3ccccc3)c3ccccc3)ccc2-c2cc3ccccc3cc21 ; CC1(C)c2cc(N(c3ccc4c(c3)-c3ccccc3C4(c3ccccc3)c3ccccc3)c3ccccc3-c3ccccc3)ccc2-c2cc3ccccc3cc21] 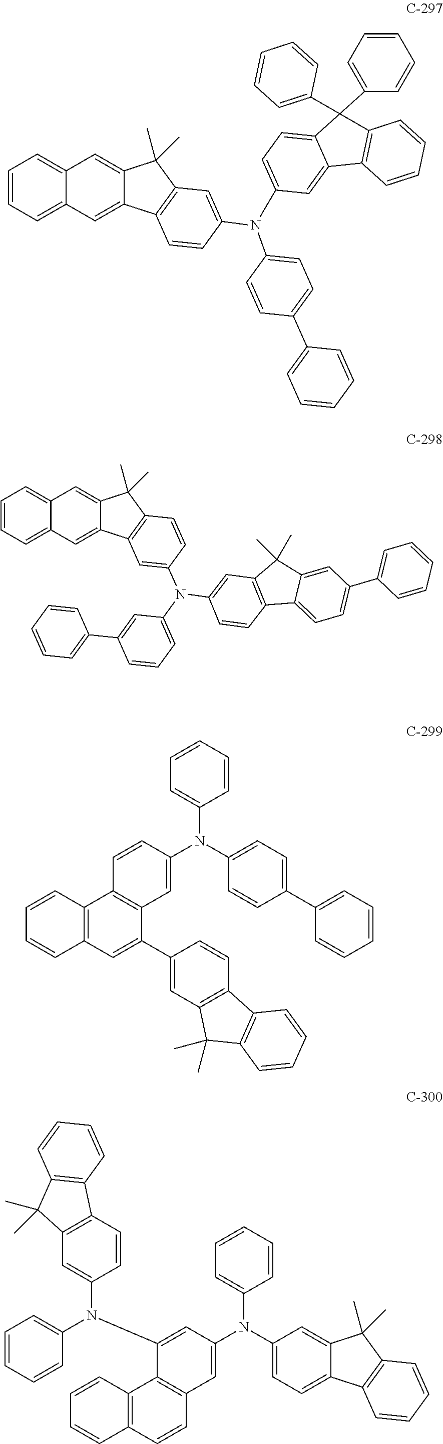 [SMILES: CC1(C)c2cc(N(c3ccc(-c4ccccc4)cc3)c3ccc4c(c3)-c3ccccc3C4(c3ccccc3)c3ccccc3)ccc2-c2cc3ccccc3cc21 ; CC1(C)c2cc(-c3ccccc3)ccc2-c2ccc(N(c3cccc(-c4ccccc4)c3)c3ccc4c(c3)-c3cc5ccccc5cc3C4(C)C)cc21 ; CC1(C)c2ccccc2-c2ccc(-c3cc4ccccc4c4ccc(N(c5ccccc5)c5ccc(-c6ccccc6)cc5)cc34)cc21 ; CC1(C)c2ccccc2-c2ccc(N(c3ccccc3)c3cc(N(c4ccccc4)c4ccc5c(c4)C(C)(C)c4ccccc4-5)c4c(ccc5ccccc54)c3)cc21] 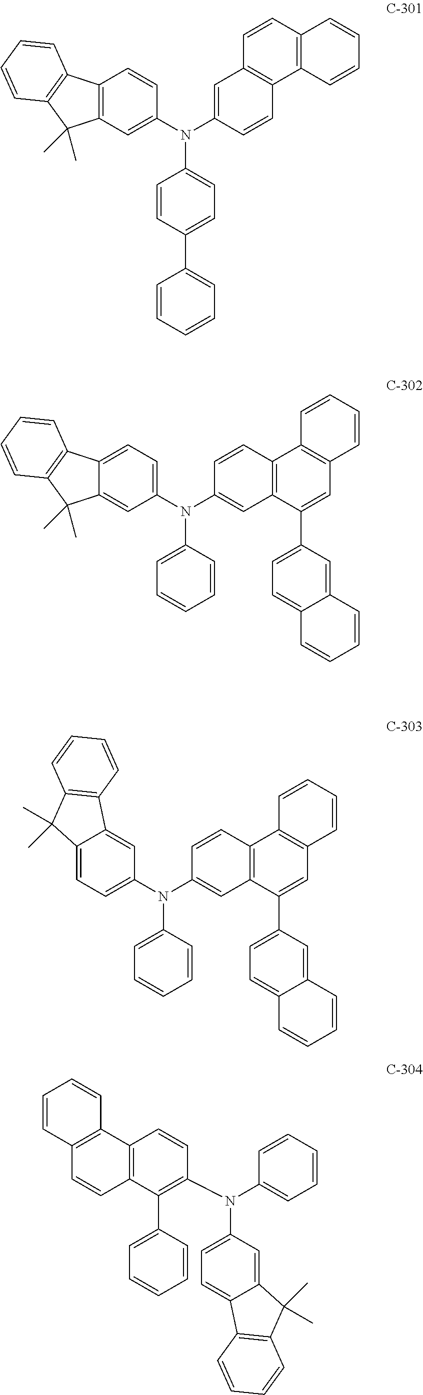 [SMILES: CC1(C)c2ccccc2-c2ccc(N(c3ccc(-c4ccccc4)cc3)c3ccc4c(ccc5ccccc54)c3)cc21 ; CC1(C)c2ccccc2-c2ccc(N(c3ccccc3)c3ccc4c(c3)c(-c3ccc5ccccc5c3)cc3ccccc34)cc21 ; CC1(C)c2ccccc2-c2cc(N(c3ccccc3)c3ccc4c(c3)c(-c3ccc5ccccc5c3)cc3ccccc34)ccc21 ; CC1(C)c2ccccc2-c2ccc(N(c3ccccc3)c3ccc4c(ccc5ccccc54)c3-c3ccccc3)cc21] 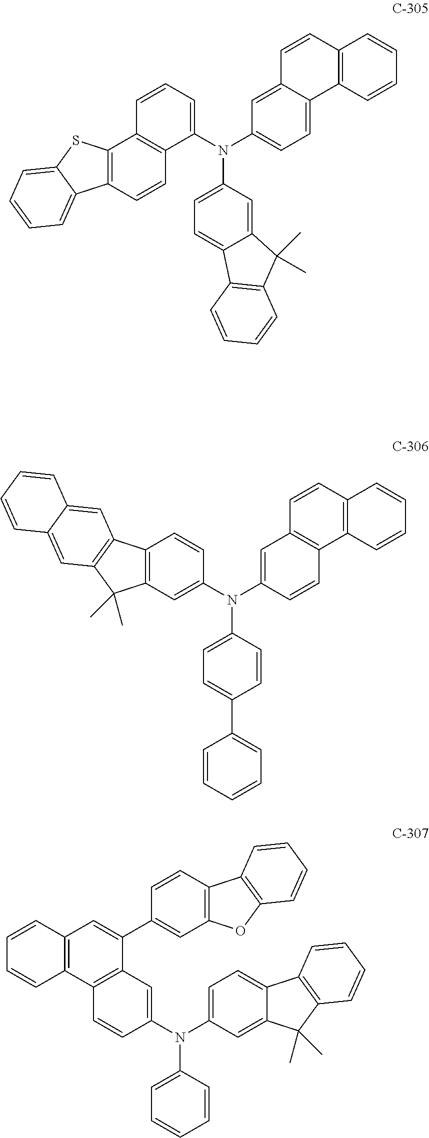 [SMILES: CC1(C)c2ccccc2-c2ccc(N(c3ccc4c(ccc5ccccc54)c3)c3cccc4c3ccc3c5ccccc5sc43)cc21 ; CC1(C)c2cc(N(c3ccc(-c4ccccc4)cc3)c3ccc4c(ccc5ccccc54)c3)ccc2-c2cc3ccccc3cc21 ; CC1(C)c2ccccc2-c2ccc(N(c3ccccc3)c3ccc4c(c3)c(-c3ccc5c(c3)oc3ccccc35)cc3ccccc34)cc21] 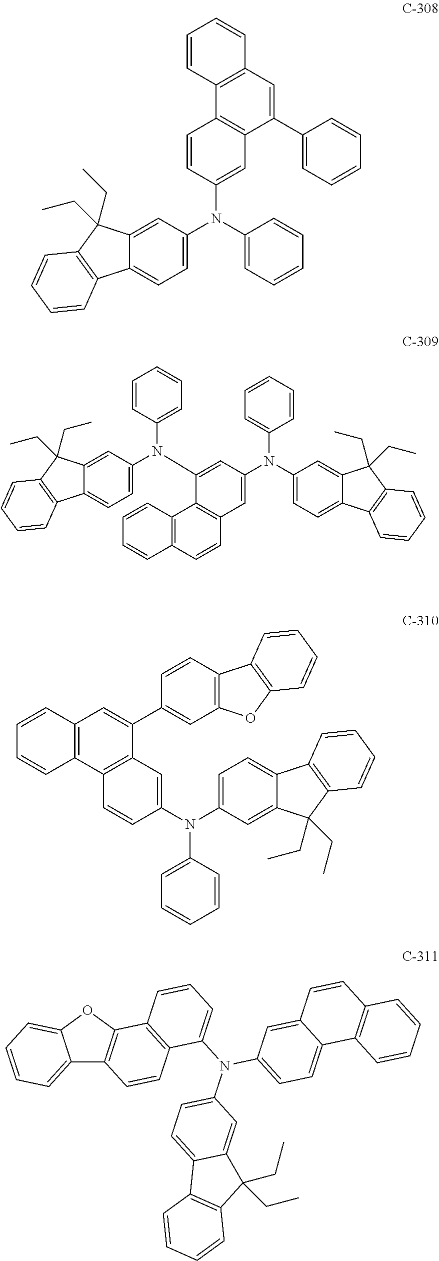 [SMILES: CCC1(CC)c2ccccc2-c2ccc(N(c3ccccc3)c3ccc4c(c3)c(-c3ccccc3)cc3ccccc34)cc21 ; CCC1(CC)c2ccccc2-c2ccc(N(c3ccccc3)c3cc(N(c4ccccc4)c4ccc5c(c4)C(CC)(CC)c4ccccc4-5)c4c(ccc5ccccc54)c3)cc21 ; CCC1(CC)c2ccccc2-c2ccc(N(c3ccccc3)c3ccc4c(c3)c(-c3ccc5c(c3)oc3ccccc35)cc3ccccc34)cc21 ; CCC1(CC)c2ccccc2-c2ccc(N(c3ccc4c(ccc5ccccc54)c3)c3cccc4c3ccc3c5ccccc5oc43)cc21] 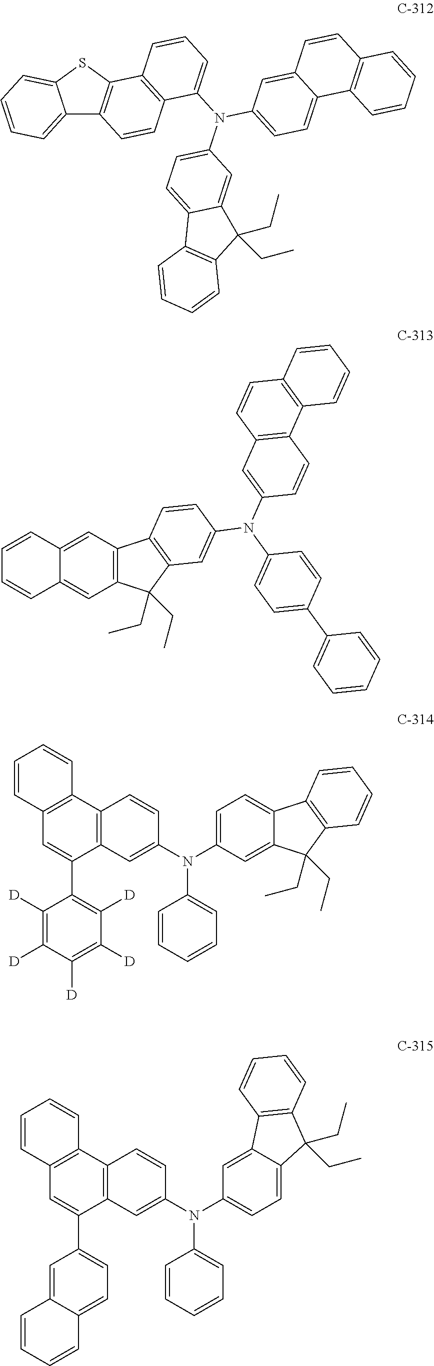 [SMILES: CCC1(CC)c2ccccc2-c2ccc(N(c3ccc4c(ccc5ccccc54)c3)c3cccc4c3ccc3c5ccccc5sc43)cc21 ; CCC1(CC)c2cc(N(c3ccc(-c4ccccc4)cc3)c3ccc4c(ccc5ccccc54)c3)ccc2-c2cc3ccccc3cc21 ; [2H]c1c([2H])c([2H])c(-c2cc3ccccc3c3ccc(N(c4ccccc4)c4ccc5c(c4)C(CC)(CC)c4ccccc4-5)cc23)c([2H])c1[2H] ; CCC1(CC)c2ccccc2-c2cc(N(c3ccccc3)c3ccc4c(c3)c(-c3ccc5ccccc5c3)cc3ccccc34)ccc21] 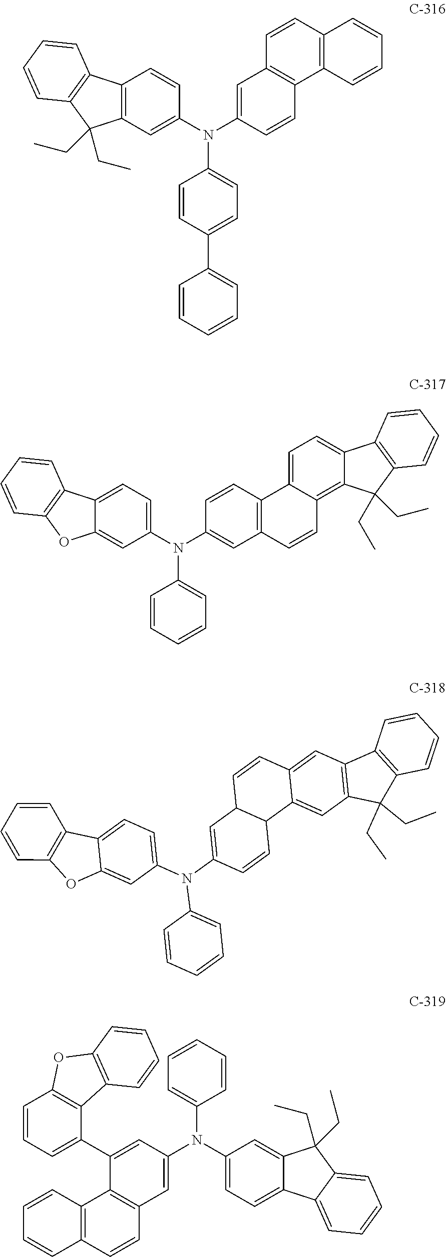 [SMILES: CCC1(CC)c2ccccc2-c2ccc(N(c3ccc(-c4ccccc4)cc3)c3ccc4c(ccc5ccccc54)c3)cc21 ; CCC1(CC)c2ccccc2-c2ccc3c(ccc4cc(N(c5ccccc5)c5ccc6c(c5)oc5ccccc56)ccc43)c21 ; CCC1(CC)c2ccccc2-c2cc3c(cc21)C1C=CC(N(c2ccccc2)c2ccc4c(c2)oc2ccccc24)=CC1C=C3 ; CCC1(CC)c2ccccc2-c2ccc(N(c3ccccc3)c3cc(-c4cccc5oc6ccccc6c45)c4c(ccc5ccccc54)c3)cc21] 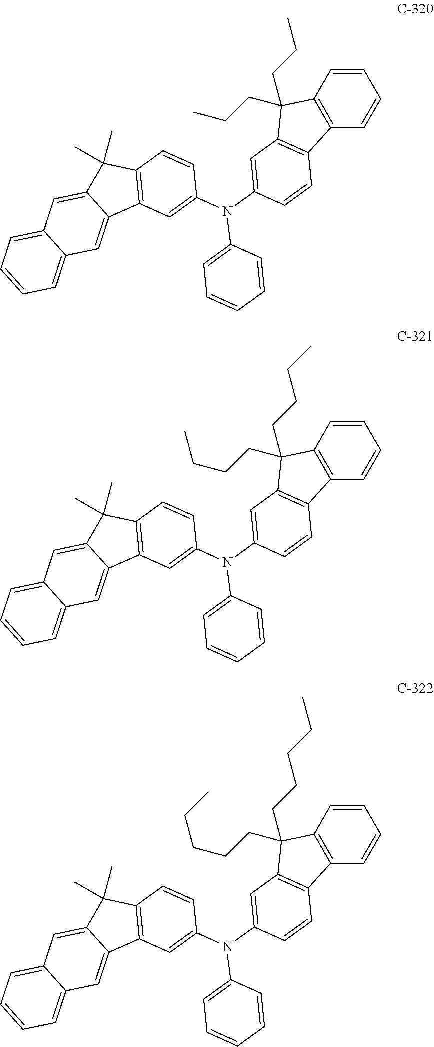 [SMILES: CCCC1(CCC)c2ccccc2-c2ccc(N(c3ccccc3)c3ccc4c(c3)-c3cc5ccccc5cc3C4(C)C)cc21 ; CCCCC1(CCCC)c2ccccc2-c2ccc(N(c3ccccc3)c3ccc4c(c3)-c3cc5ccccc5cc3C4(C)C)cc21 ; CCCCCC1(CCCCC)c2ccccc2-c2ccc(N(c3ccccc3)c3ccc4c(c3)-c3cc5ccccc5cc3C4(C)C)cc21] 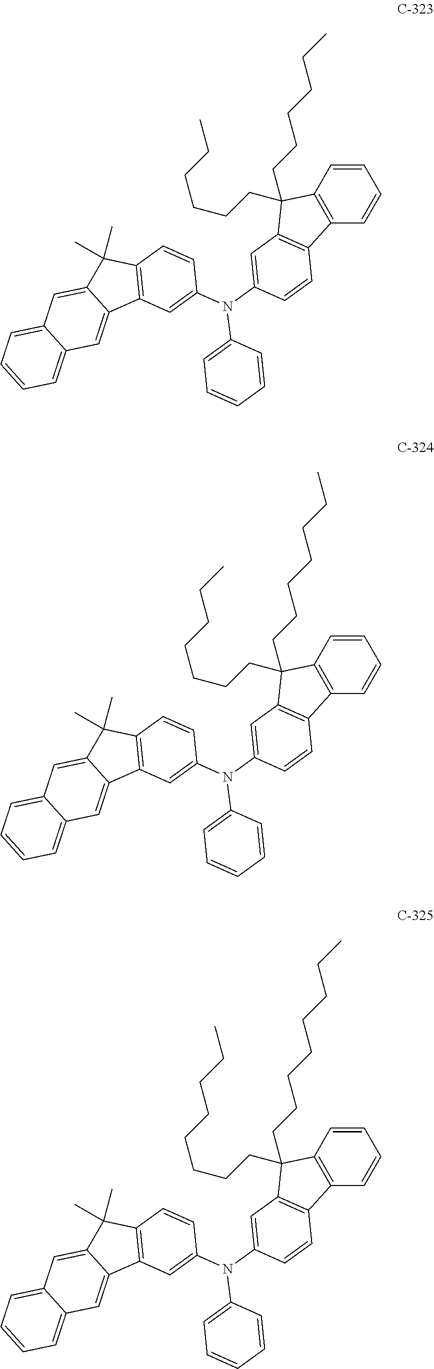 [SMILES: CCCCCCC1(CCCCCC)c2ccccc2-c2ccc(N(c3ccccc3)c3ccc4c(c3)-c3cc5ccccc5cc3C4(C)C)cc21 ; CCCCCCCC1(CCCCCCC)c2ccccc2-c2ccc(N(c3ccccc3)c3ccc4c(c3)-c3cc5ccccc5cc3C4(C)C)cc21 ; CCCCCCCCC1(CCCCCCCC)c2ccccc2-c2ccc(N(c3ccccc3)c3ccc4c(c3)-c3cc5ccccc5cc3C4(C)C)cc21] 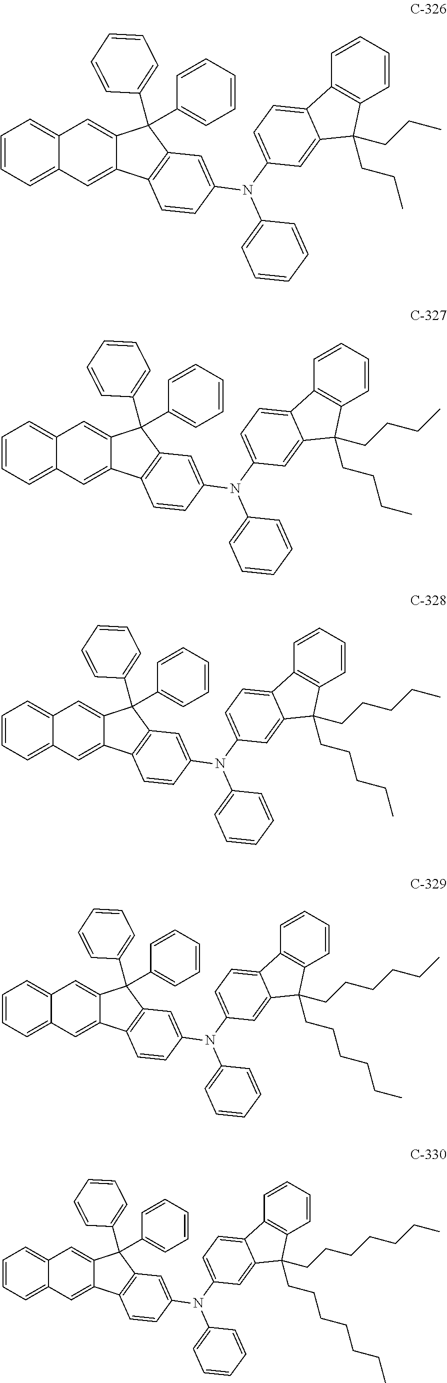 [SMILES: CCCC1(CCC)c2ccccc2-c2ccc(N(c3ccccc3)c3ccc4c(c3)C(c3ccccc3)(c3ccccc3)c3cc5ccccc5cc3-4)cc21 ; CCCCC1(CCCC)c2ccccc2-c2ccc(N(c3ccccc3)c3ccc4c(c3)C(c3ccccc3)(c3ccccc3)c3cc5ccccc5cc3-4)cc21 ; CCCCCC1(CCCCC)c2ccccc2-c2ccc(N(c3ccccc3)c3ccc4c(c3)C(c3ccccc3)(c3ccccc3)c3cc5ccccc5cc3-4)cc21 ; CCCCCCC1(CCCCCC)c2ccccc2-c2ccc(N(c3ccccc3)c3ccc4c(c3)C(c3ccccc3)(c3ccccc3)c3cc5ccccc5cc3-4)cc21 ; CCCCCCCC1(CCCCCCC)c2ccccc2-c2ccc(N(c3ccccc3)c3ccc4c(c3)C(c3ccccc3)(c3ccccc3)c3cc5ccccc5cc3-4)cc21] 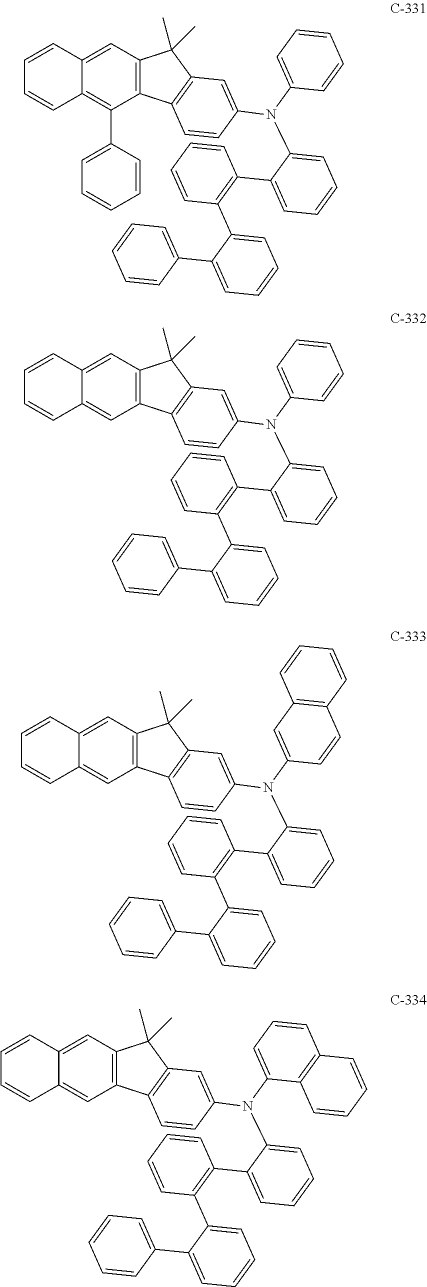 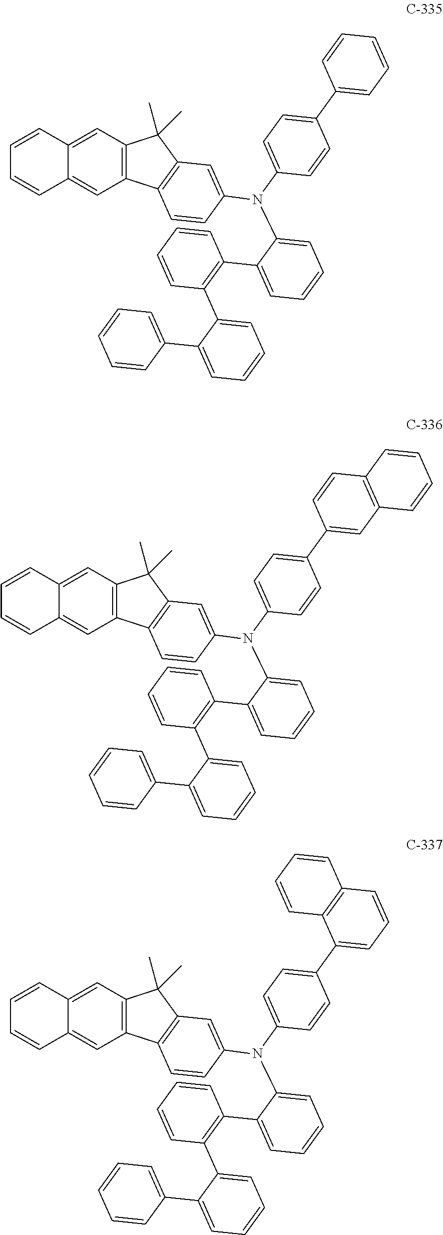 [SMILES: CC1(C)c2cc(N(c3ccc(-c4ccccc4)cc3)c3ccccc3-c3ccccc3-c3ccccc3-c3ccccc3)ccc2-c2cc3ccccc3cc21 ; CC1(C)c2cc(N(c3ccc(-c4ccc5ccccc5c4)cc3)c3ccccc3-c3ccccc3-c3ccccc3-c3ccccc3)ccc2-c2cc3ccccc3cc21 ; CC1(C)c2cc(N(c3ccc(-c4cccc5ccccc45)cc3)c3ccccc3-c3ccccc3-c3ccccc3-c3ccccc3)ccc2-c2cc3ccccc3cc21] 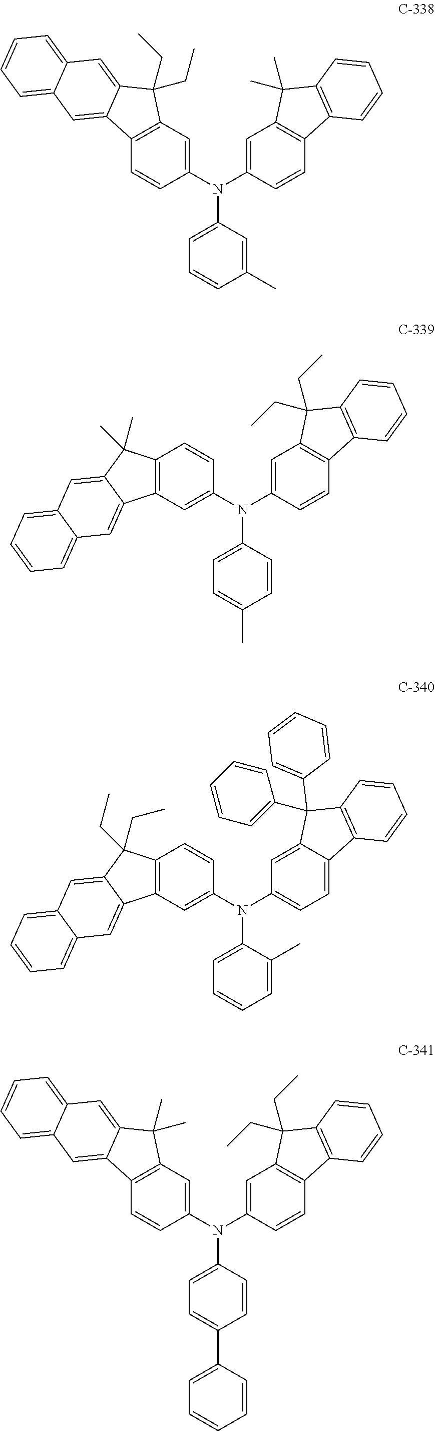 [SMILES: CCC1(CC)c2cc(N(c3cccc(C)c3)c3ccc4c(c3)C(C)(C)c3ccccc3-4)ccc2-c2cc3ccccc3cc21 ; CCC1(CC)c2ccccc2-c2ccc(N(c3ccc(C)cc3)c3ccc4c(c3)-c3cc5ccccc5cc3C4(C)C)cc21 ; CCC1(CC)c2ccc(N(c3ccc4c(c3)C(c3ccccc3)(c3ccccc3)c3ccccc3-4)c3ccccc3C)cc2-c2cc3ccccc3cc21 ; CCC1(CC)c2ccccc2-c2ccc(N(c3ccc(-c4ccccc4)cc3)c3ccc4c(c3)C(C)(C)c3cc5ccccc5cc3-4)cc21] 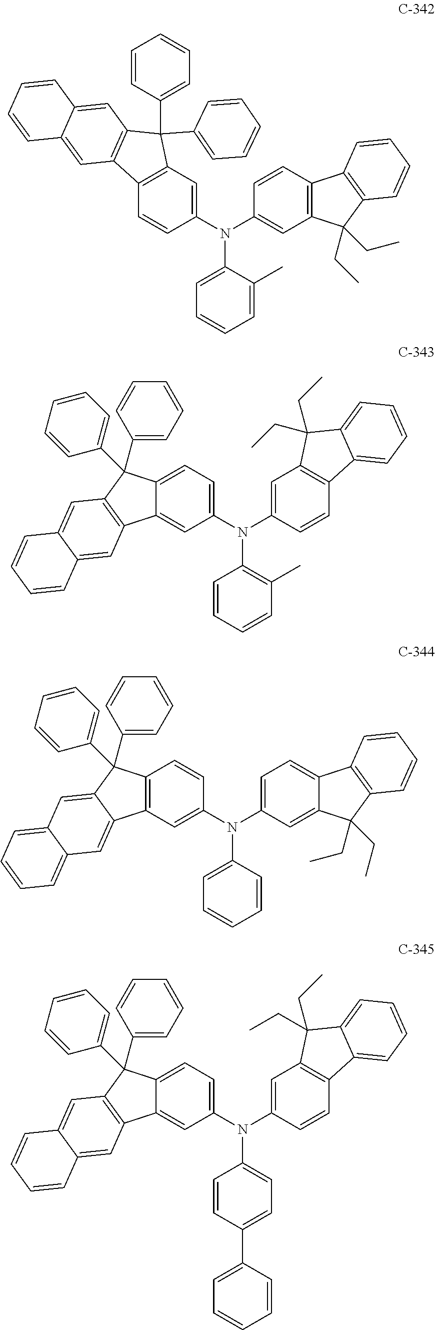 [SMILES: CCC1(CC)c2ccccc2-c2ccc(N(c3ccc4c(c3)C(c3ccccc3)(c3ccccc3)c3cc5ccccc5cc3-4)c3ccccc3C)cc21 ; CCC1(CC)c2ccccc2-c2ccc(N(c3ccc4c(c3)-c3cc5ccccc5cc3C4(c3ccccc3)c3ccccc3)c3ccccc3C)cc21 ; CCC1(CC)c2ccccc2-c2ccc(N(c3ccccc3)c3ccc4c(c3)-c3cc5ccccc5cc3C4(c3ccccc3)c3ccccc3)cc21 ; CCC1(CC)c2ccccc2-c2ccc(N(c3ccc(-c4ccccc4)cc3)c3ccc4c(c3)-c3cc5ccccc5cc3C4(c3ccccc3)c3ccccc3)cc21] 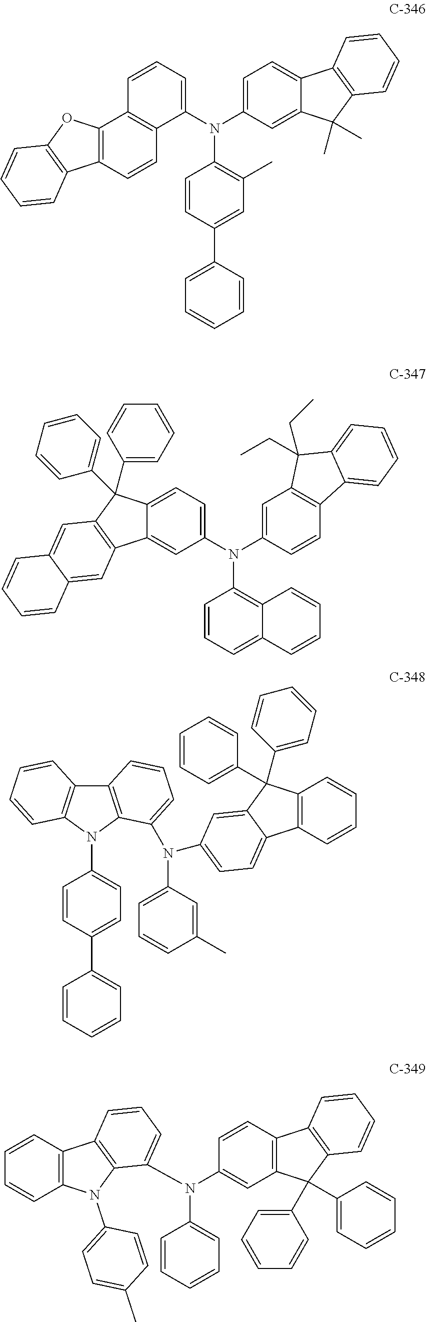 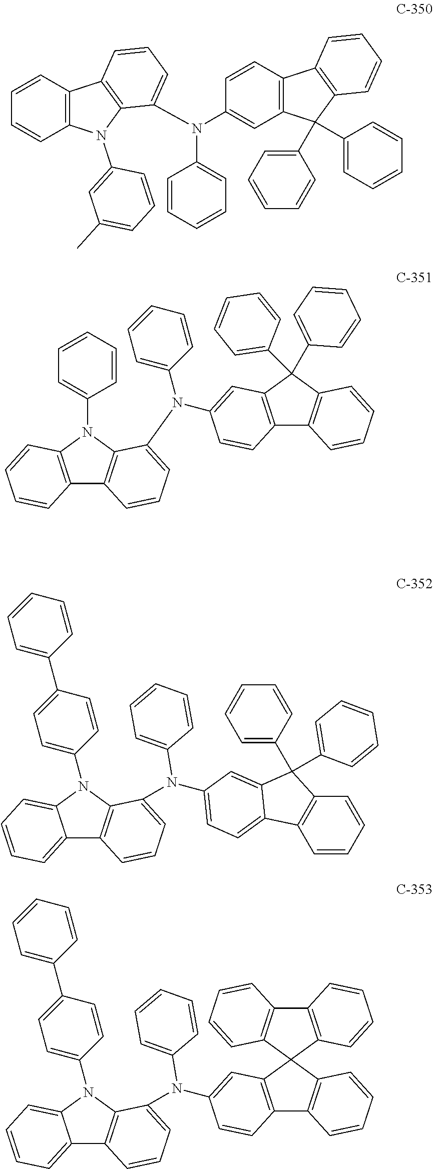 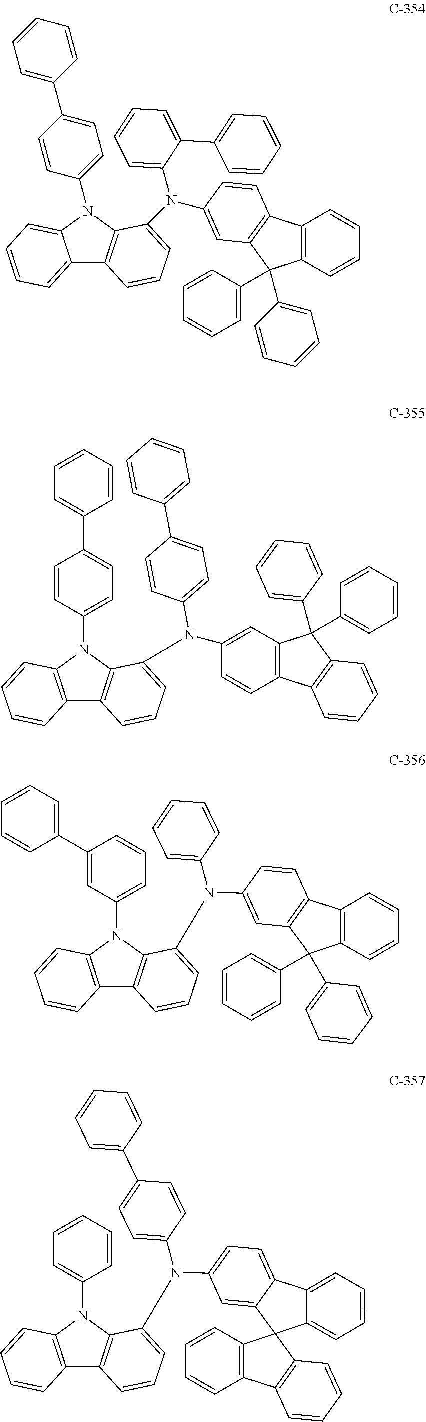 [SMILES: c1ccc(-c2ccc(-n3c4ccccc4c4cccc(N(c5ccc6c(c5)C(c5ccccc5)(c5ccccc5)c5ccccc5-6)c5ccccc5-c5ccccc5)c43)cc2)cc1 ; c1ccc(-c2ccc(N(c3ccc4c(c3)C(c3ccccc3)(c3ccccc3)c3ccccc3-4)c3cccc4c5ccccc5n(-c5ccc(-c6ccccc6)cc5)c34)cc2)cc1 ; c1ccc(-c2cccc(-n3c4ccccc4c4cccc(N(c5ccccc5)c5ccc6c(c5)C(c5ccccc5)(c5ccccc5)c5ccccc5-6)c43)c2)cc1 ; c1ccc(-c2ccc(N(c3ccc4c(c3)C3(c5ccccc5-c5ccccc53)c3ccccc3-4)c3cccc4c5ccccc5n(-c5ccccc5)c34)cc2)cc1] 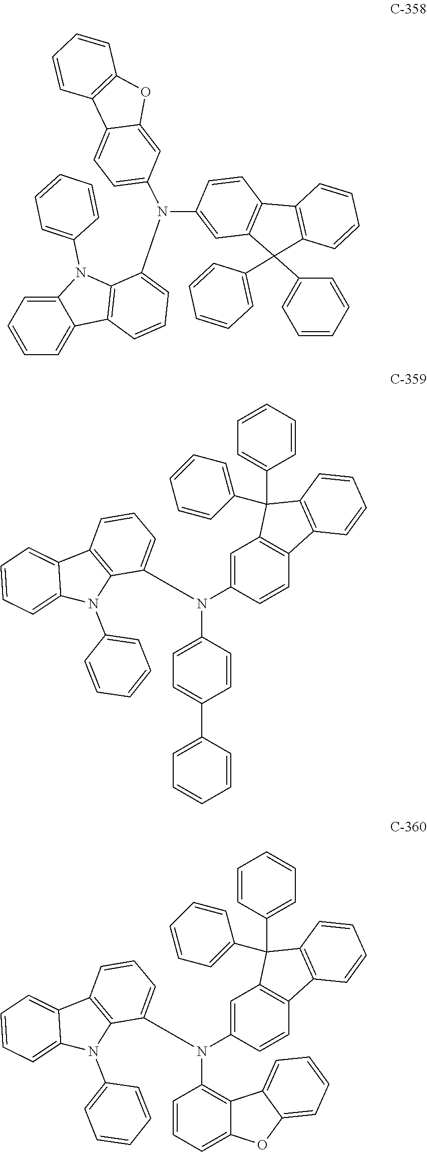 [SMILES: c1ccc(-n2c3ccccc3c3cccc(N(c4ccc5c(c4)C(c4ccccc4)(c4ccccc4)c4ccccc4-5)c4ccc5c(c4)oc4ccccc45)c32)cc1 ; c1ccc(-c2ccc(N(c3ccc4c(c3)C(c3ccccc3)(c3ccccc3)c3ccccc3-4)c3cccc4c5ccccc5n(-c5ccccc5)c34)cc2)cc1 ; c1ccc(-n2c3ccccc3c3cccc(N(c4ccc5c(c4)C(c4ccccc4)(c4ccccc4)c4ccccc4-5)c4cccc5oc6ccccc6c45)c32)cc1] 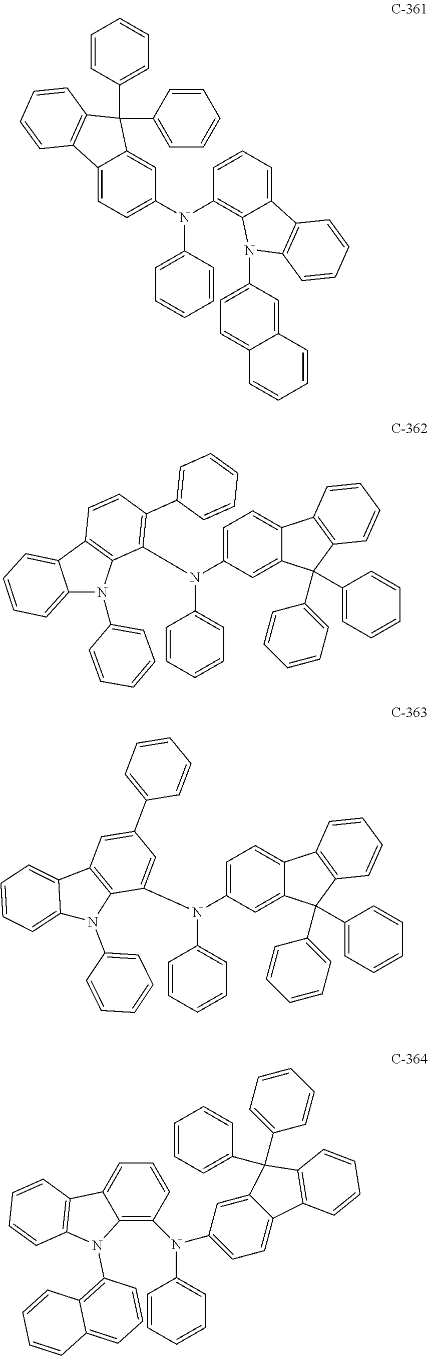 [SMILES: c1ccc(N(c2ccc3c(c2)C(c2ccccc2)(c2ccccc2)c2ccccc2-3)c2cccc3c4ccccc4n(-c4ccc5ccccc5c4)c23)cc1 ; c1ccc(-c2ccc3c4ccccc4n(-c4ccccc4)c3c2N(c2ccccc2)c2ccc3c(c2)C(c2ccccc2)(c2ccccc2)c2ccccc2-3)cc1 ; c1ccc(-c2cc(N(c3ccccc3)c3ccc4c(c3)C(c3ccccc3)(c3ccccc3)c3ccccc3-4)c3c(c2)c2ccccc2n3-c2ccccc2)cc1 ; c1ccc(N(c2ccc3c(c2)C(c2ccccc2)(c2ccccc2)c2ccccc2-3)c2cccc3c4ccccc4n(-c4cccc5ccccc45)c23)cc1] 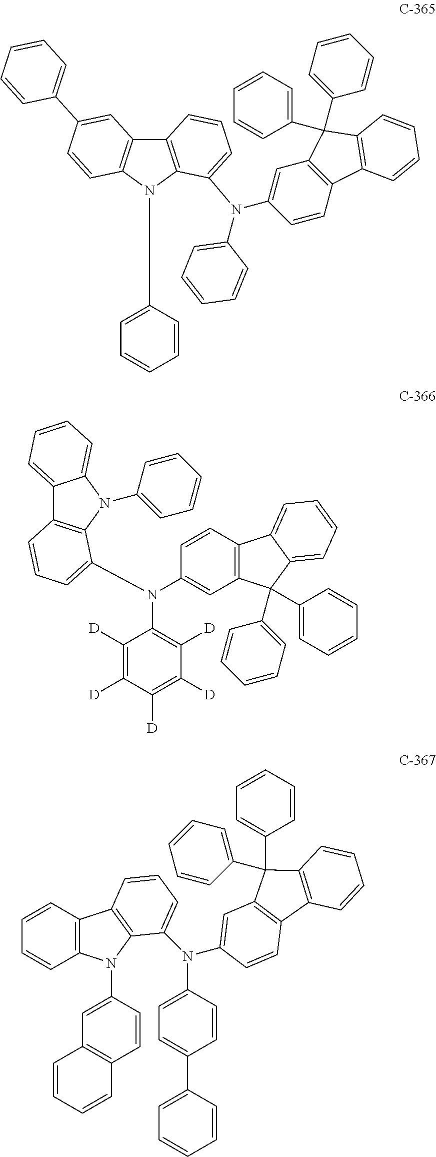 [SMILES: c1ccc(-c2ccc3c(c2)c2cccc(N(c4ccccc4)c4ccc5c(c4)C(c4ccccc4)(c4ccccc4)c4ccccc4-5)c2n3-c2ccccc2)cc1 ; [2H]c1c([2H])c([2H])c(N(c2ccc3c(c2)C(c2ccccc2)(c2ccccc2)c2ccccc2-3)c2cccc3c4ccccc4n(-c4ccccc4)c23)c([2H])c1[2H] ; c1ccc(-c2ccc(N(c3ccc4c(c3)C(c3ccccc3)(c3ccccc3)c3ccccc3-4)c3cccc4c5ccccc5n(-c5ccc6ccccc6c5)c34)cc2)cc1] 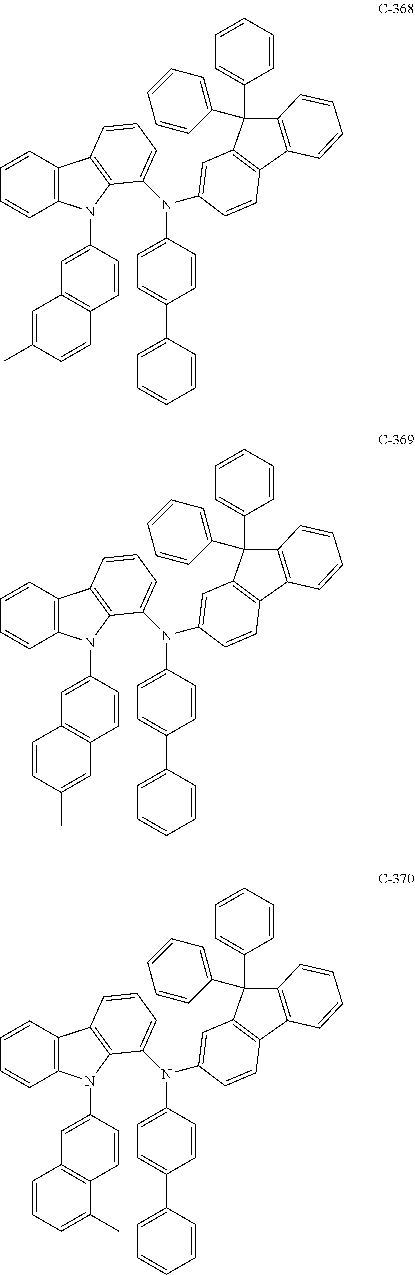 [SMILES: Cc1ccc2ccc(-n3c4ccccc4c4cccc(N(c5ccc(-c6ccccc6)cc5)c5ccc6c(c5)C(c5ccccc5)(c5ccccc5)c5ccccc5-6)c43)cc2c1 ; Cc1ccc2cc(-n3c4ccccc4c4cccc(N(c5ccc(-c6ccccc6)cc5)c5ccc6c(c5)C(c5ccccc5)(c5ccccc5)c5ccccc5-6)c43)ccc2c1 ; Cc1cccc2cc(-n3c4ccccc4c4cccc(N(c5ccc(-c6ccccc6)cc5)c5ccc6c(c5)C(c5ccccc5)(c5ccccc5)c5ccccc5-6)c43)ccc12] 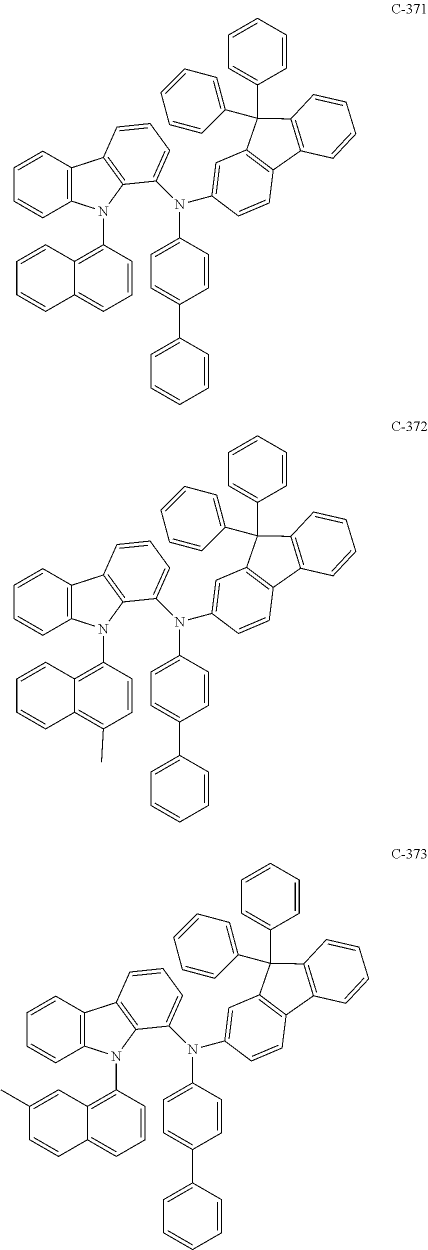 [SMILES: c1ccc(-c2ccc(N(c3ccc4c(c3)C(c3ccccc3)(c3ccccc3)c3ccccc3-4)c3cccc4c5ccccc5n(-c5cccc6ccccc56)c34)cc2)cc1 ; Cc1ccc(-n2c3ccccc3c3cccc(N(c4ccc(-c5ccccc5)cc4)c4ccc5c(c4)C(c4ccccc4)(c4ccccc4)c4ccccc4-5)c32)c2ccccc12 ; Cc1ccc2cccc(-n3c4ccccc4c4cccc(N(c5ccc(-c6ccccc6)cc5)c5ccc6c(c5)C(c5ccccc5)(c5ccccc5)c5ccccc5-6)c43)c2c1] 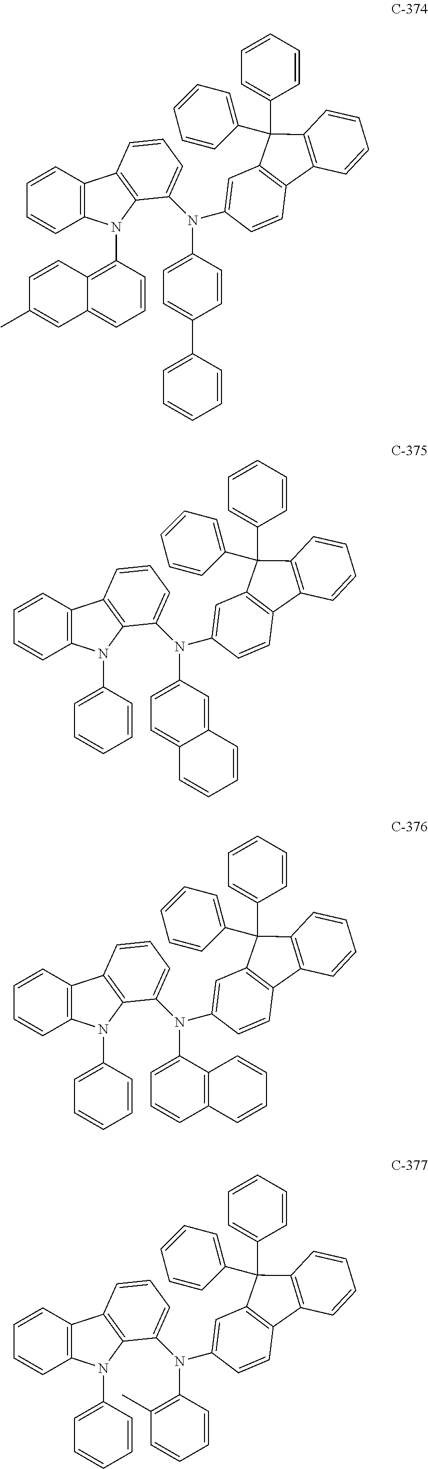 [SMILES: Cc1ccc2c(-n3c4ccccc4c4cccc(N(c5ccc(-c6ccccc6)cc5)c5ccc6c(c5)C(c5ccccc5)(c5ccccc5)c5ccccc5-6)c43)cccc2c1 ; c1ccc(-n2c3ccccc3c3cccc(N(c4ccc5c(c4)C(c4ccccc4)(c4ccccc4)c4ccccc4-5)c4ccc5ccccc5c4)c32)cc1 ; c1ccc(-n2c3ccccc3c3cccc(N(c4ccc5c(c4)C(c4ccccc4)(c4ccccc4)c4ccccc4-5)c4cccc5ccccc45)c32)cc1 ; Cc1ccccc1N(c1ccc2c(c1)C(c1ccccc1)(c1ccccc1)c1ccccc1-2)c1cccc2c3ccccc3n(-c3ccccc3)c12] 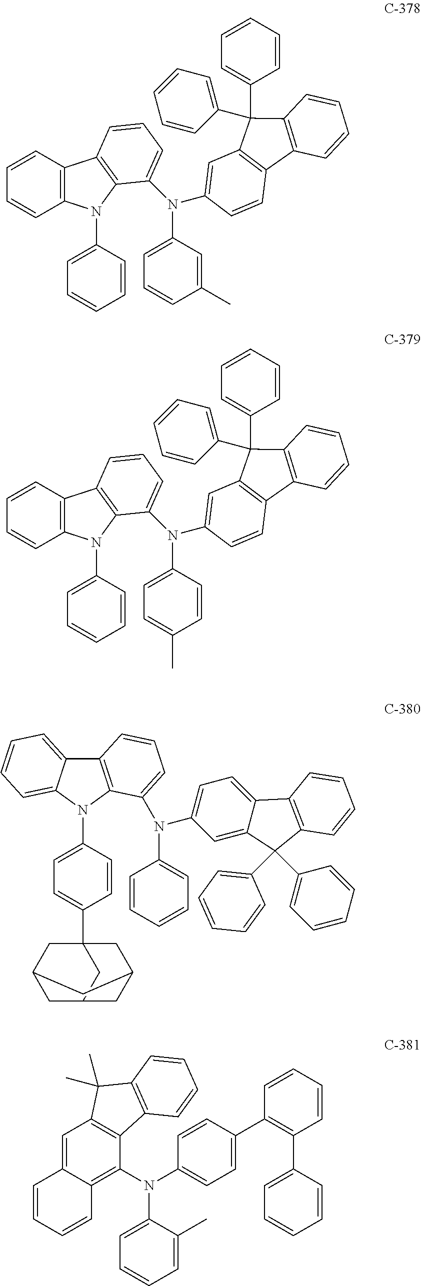 [SMILES: Cc1cccc(N(c2ccc3c(c2)C(c2ccccc2)(c2ccccc2)c2ccccc2-3)c2cccc3c4ccccc4n(-c4ccccc4)c23)c1 ; Cc1ccc(N(c2ccc3c(c2)C(c2ccccc2)(c2ccccc2)c2ccccc2-3)c2cccc3c4ccccc4n(-c4ccccc4)c23)cc1 ; c1ccc(N(c2ccc3c(c2)C(c2ccccc2)(c2ccccc2)c2ccccc2-3)c2cccc3c4ccccc4n(-c4ccc(C56CC7CC(CC(C7)C5)C6)cc4)c23)cc1 ; Cc1ccccc1N(c1ccc(-c2ccccc2-c2ccccc2)cc1)c1c2c(cc3ccccc13)C(C)(C)c1ccccc1-2] 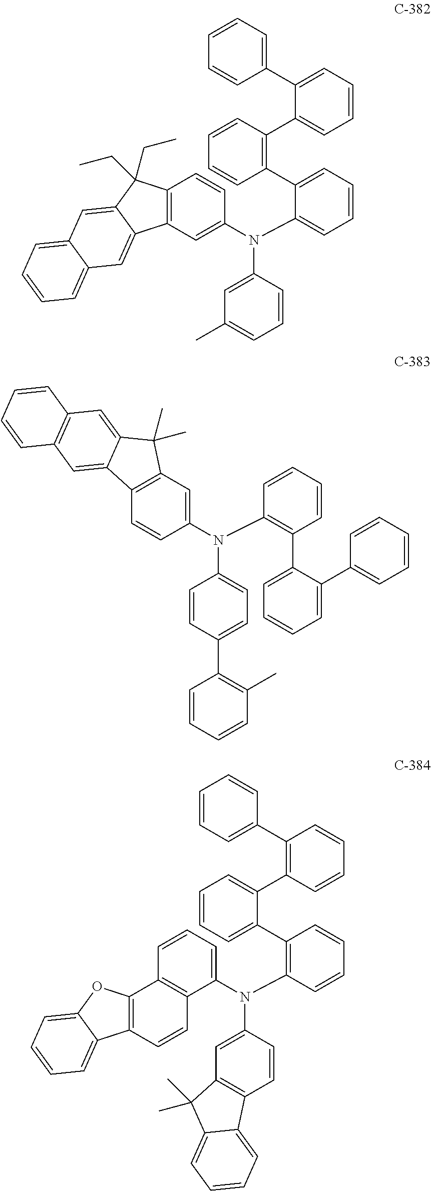 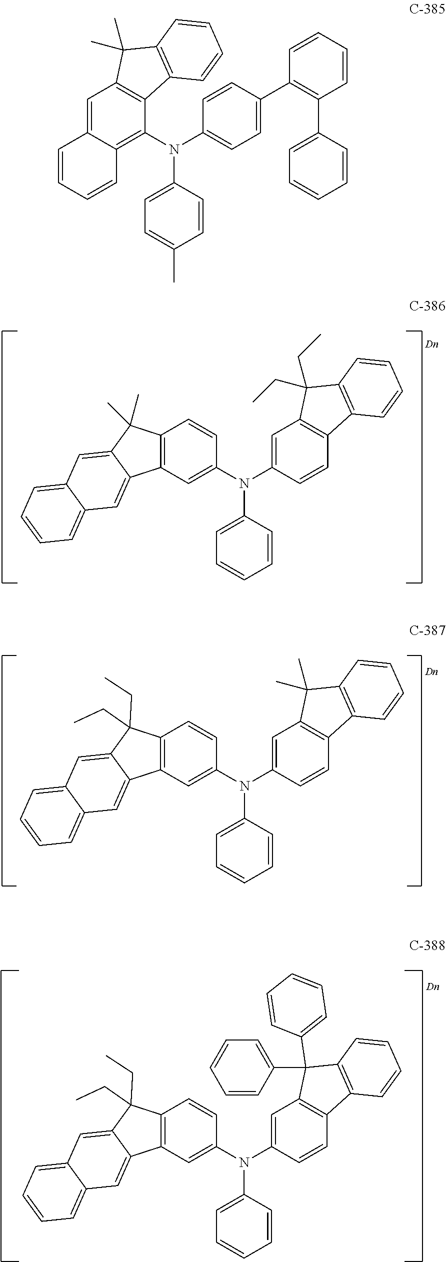 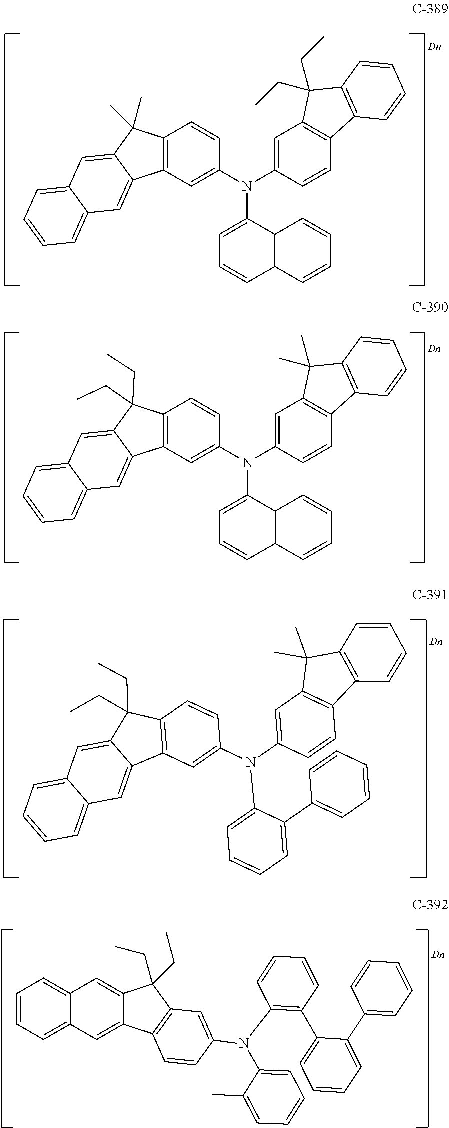 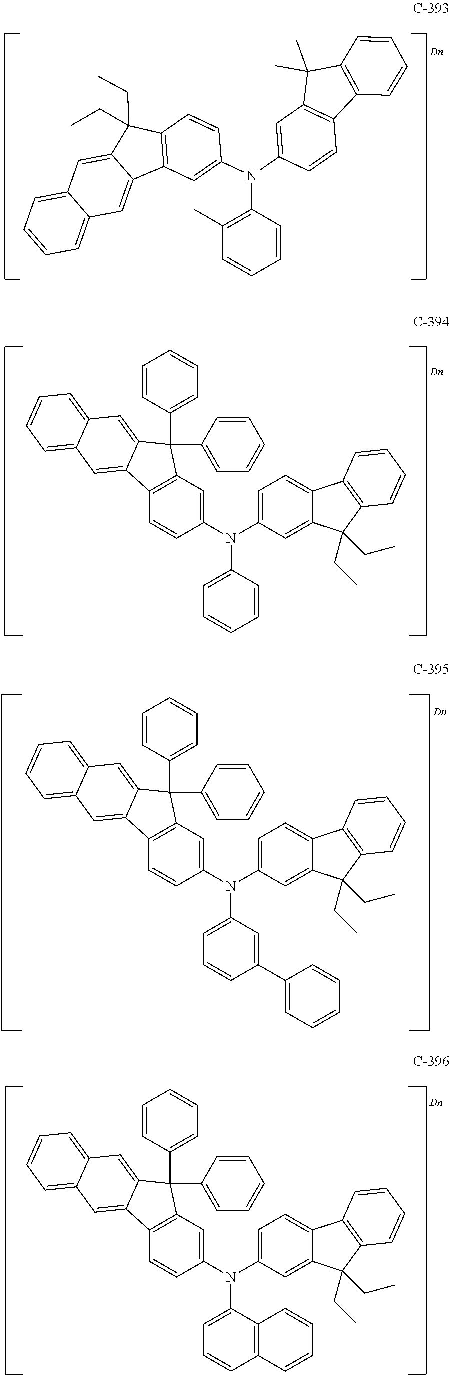 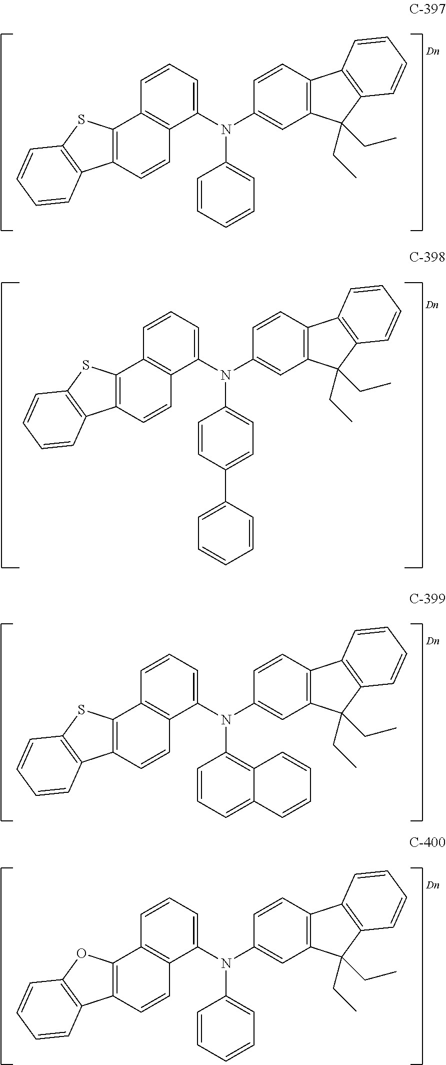 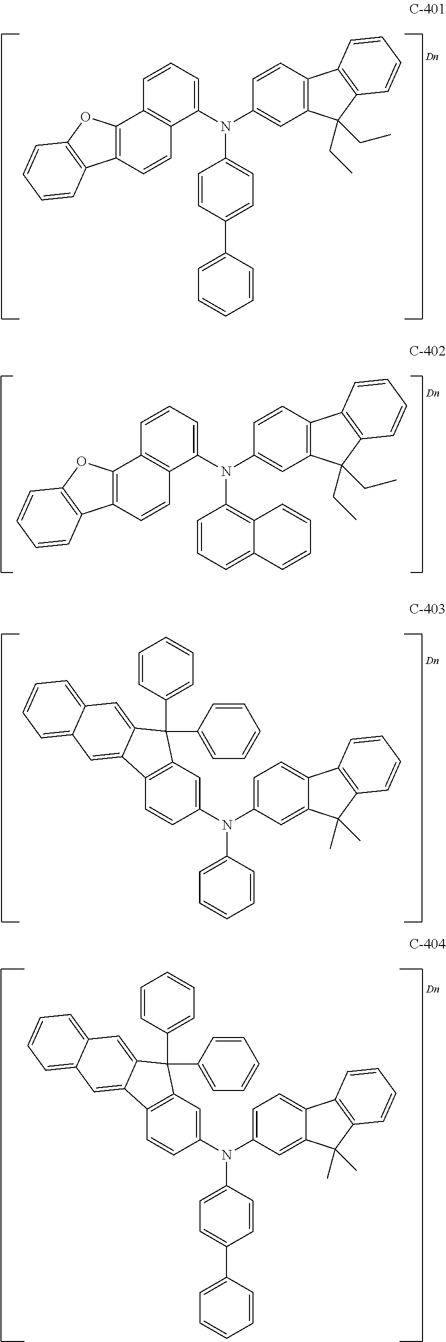 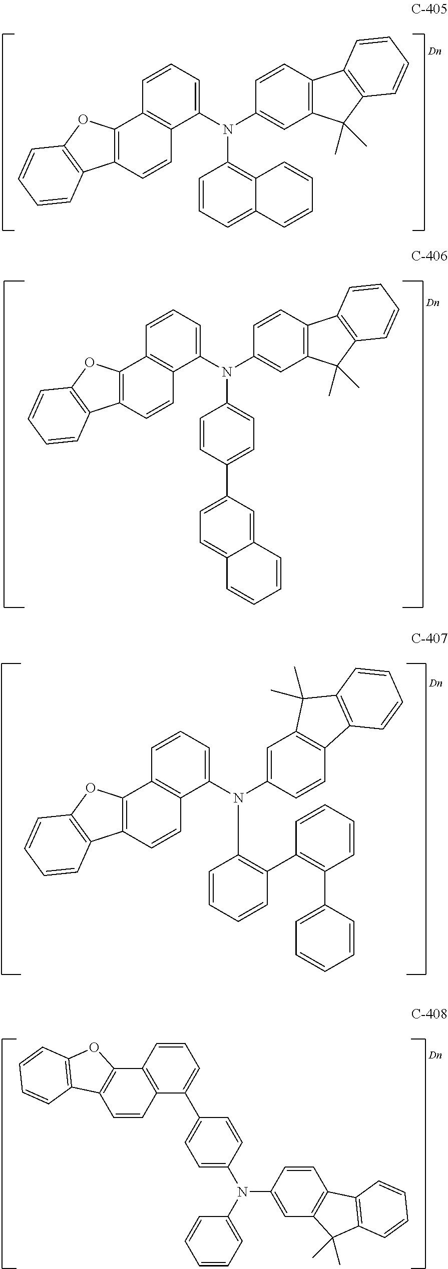 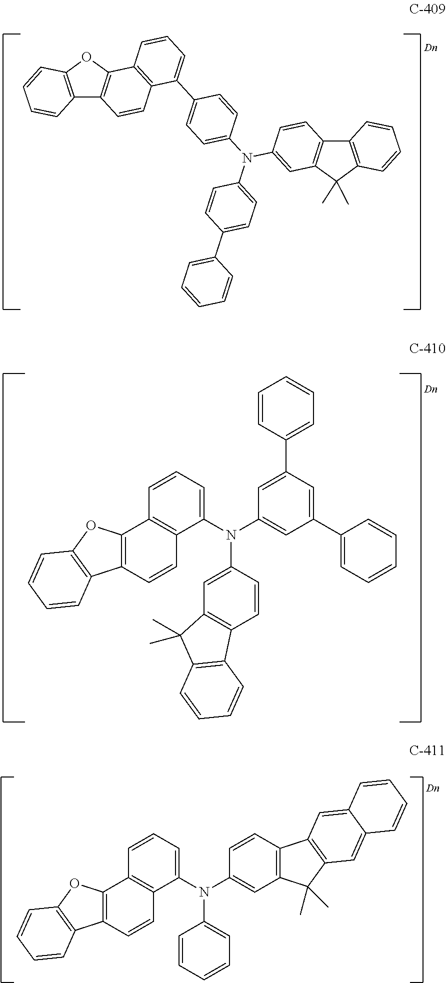 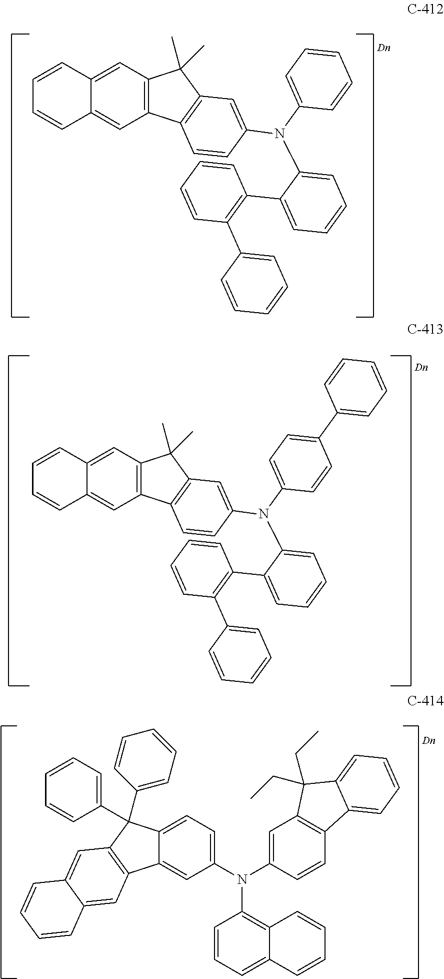 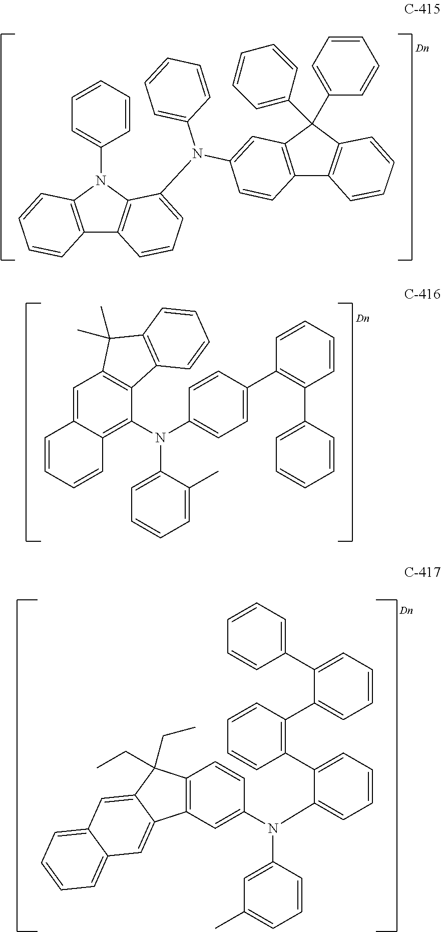 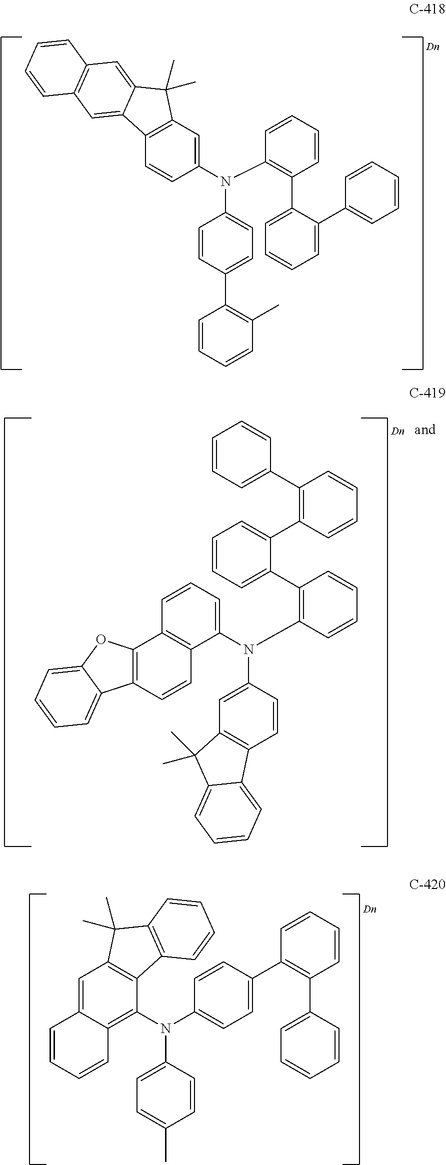
wherein in the compounds above, Dn represents that n number of hydrogens are replaced with deuterium, and n represents an integer of 1 or more, which is an integer of 1 to the maximum number of hydrogens in the compound.

5. The organic electroluminescent device according to claim 2, wherein the substituent(s) of the substituted alkyl, the substituted alkenyl, the substituted aryl(ene), the substituted heteroaryl(ene), the substituted cycloalkyl, and the substituted alkoxy each independently are at least one selected from the group consisting of deuterium; a halogen; a cyano; a carboxyl; a nitro; a hydroxy; a phosphine oxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (C6-C30)aryl unsubstituted or substituted with one or more of (C1-C30)alkyl and di(C6-C30)arylamino; a (3- to 30-membered)heteroaryl unsubstituted or substituted with one or more of (C6-C30)aryl; a tri(C1-C30)alkylsilyl; a tri(C6-C30)arylsilyl; a di(C1-C30)alkyl(C6-C30)arylsilyl; a (C1-C30)alkyldi(C6-C30)arylsilyl; a fused ring group of a (C3-C30) aliphatic ring(s) and a (C6-C30) aromatic ring(s); an amino; a mono- or di(C1-C30)alkylamino; a mono- or di(C2-C30)alkenylamino; a mono- or di(C6-C30)arylamino unsubstituted or substituted with a (C1-C30)alkyl(s); a mono- or di(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

6. The organic electroluminescent device according to claim 3, wherein the light-emitting layer emits any one color of blue, green, or red, with a proviso that the light-emitting layer emits the blue or green color, the case of R₂ or R₇ of Formula 1-1 is -L₁-NAr₃Ar₄ is excluded.

7. The organic electroluminescent device according to claim 1, wherein the third hole transport layer comprises the compound represented by the following Formula 2: 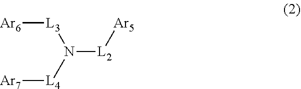
wherein in Formula 2,
L₂ to L₄ each independently represent a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene; and
Ar₅ to Ar₇ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di(C1-C30)alkylamino, a substituted or unsubstituted mono- or di(C6-C30)arylamino, or a substituted or unsubstituted (C1-C30)alkyl(C6-C30)arylamino;
with a proviso that the case where L₂ to L₄ are all single bonds, and Ar₅ to Ar₇ are all hydrogen is excluded.

8. The organic electroluminescent device according to claim 7, wherein the Ar₅ in Formula 2 is represented by any one of the following Formulas 2-1 to 2-6: 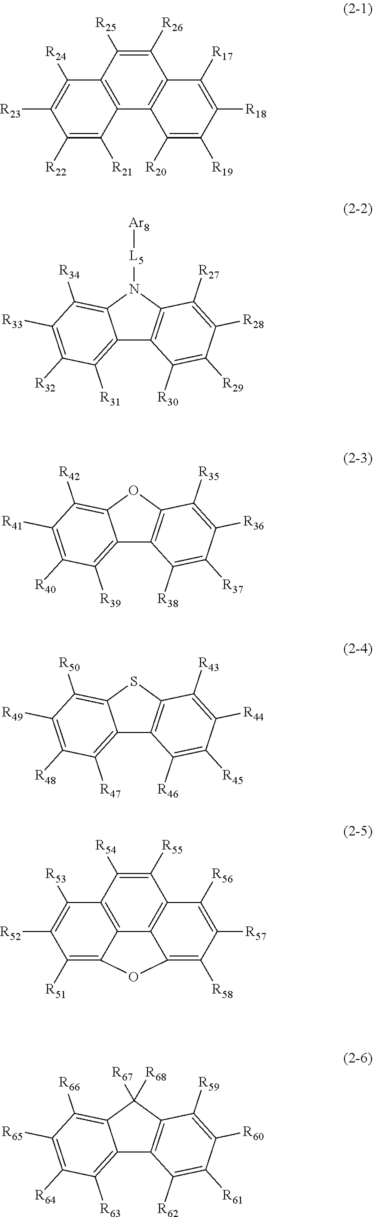
wherein in Formulas 2-1 to 2-6,
L₅ represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;
Ar₈ links to L₂ of Formula 2, or represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di(C1-C30)alkylamino, a substituted or unsubstituted mono- or di(C6-C30)arylamino, or a substituted or unsubstituted (C1-C30)alkyl(C6-C30)arylamino; and
R₁₇ to R₆₈ are linked to L₂ of Formula 2, or each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di(C1-C30)alkylamino, a substituted or unsubstituted mono- or di(C6-C30)arylamino, or a substituted or unsubstituted (C1-C30)alkyl(C6-C30)arylamino; or may be linked to an adjacent substituent(s) to form a ring(s);
with a proviso that at least one of Ar₈ and R₁₇ to R₆₈ is linked to L₂ of Formula 2.

9. The organic electroluminescent device according to claim 8, wherein Formula 2-3 is represented by any one of the following Formulas 2-3-a to 2-3-c, or Formula 2-4 is represented by any one of the following Formulas 2-4-a to 2-4-c: 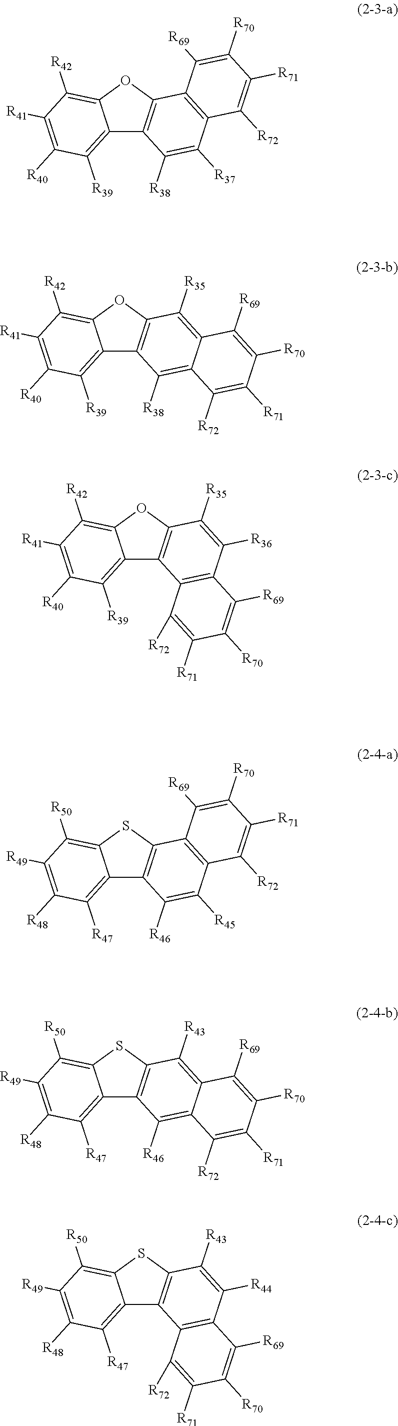
wherein in Formulas 2-3-a to 2-3-c and Formulas 2-4-a to 2-4-c,
R₃₅ to R₅₀ are as defined in claim 8; and
R₆₉ to R₇₂ are linked to L₂ of Formula 2, or each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di(C1-C30)alkylamino, a substituted or unsubstituted mono- or di(C6-C30)arylamino, or a substituted or unsubstituted (C1-C30)alkyl(C6-C30)arylamino; or may be linked to an adjacent substituent(s) to form a ring(s);
with a proviso that at least one of R₃₅ to R₅₀, and R₆₉ to R₇₂ is linked to L₂ of Formula 2.

10. The organic electroluminescent device according to claim 7, wherein the compound represented by Formula 2 is at least one selected from the following compounds: 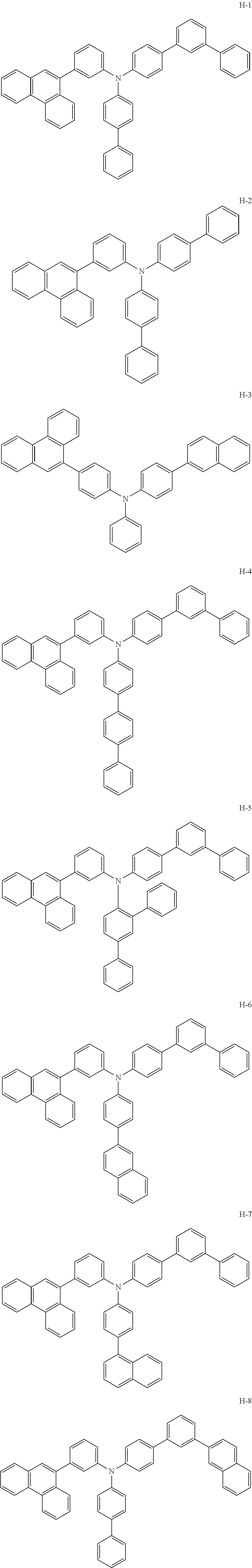 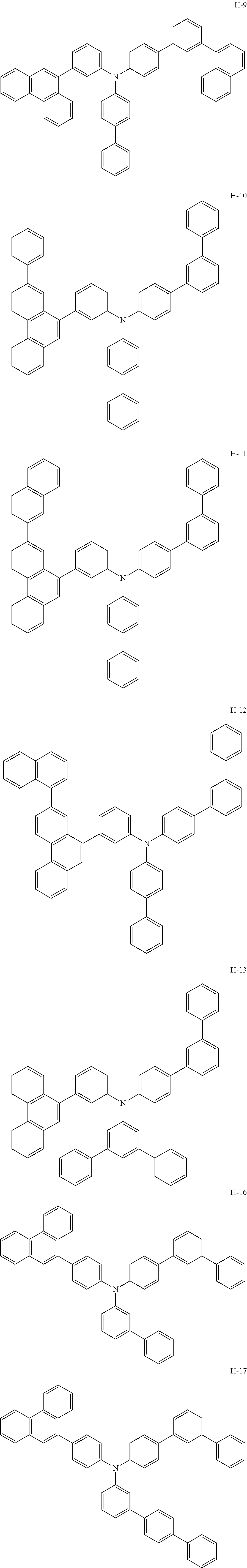 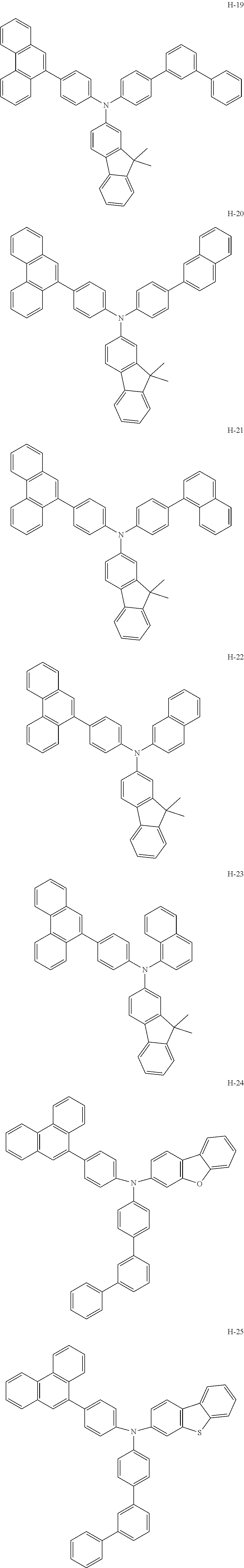 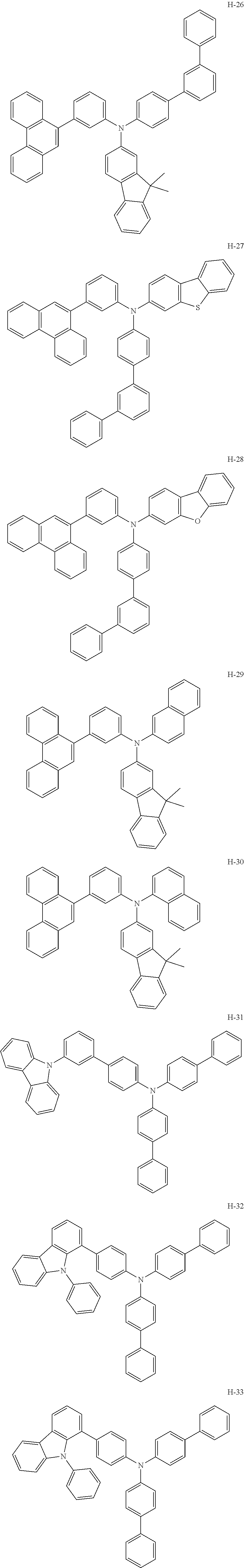 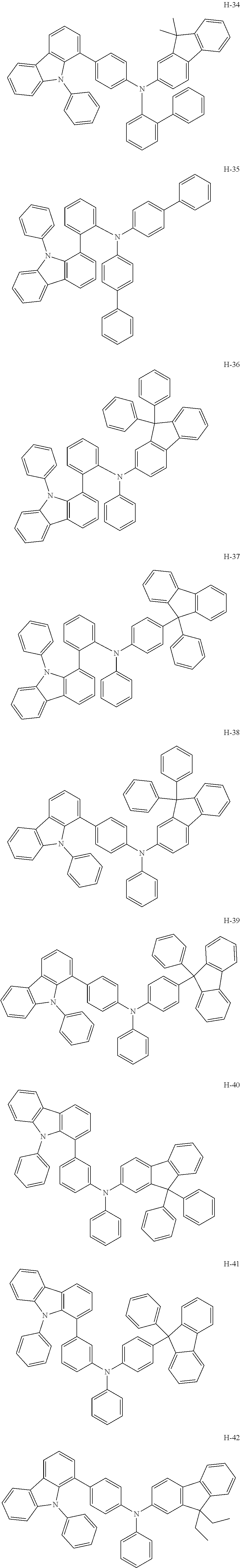 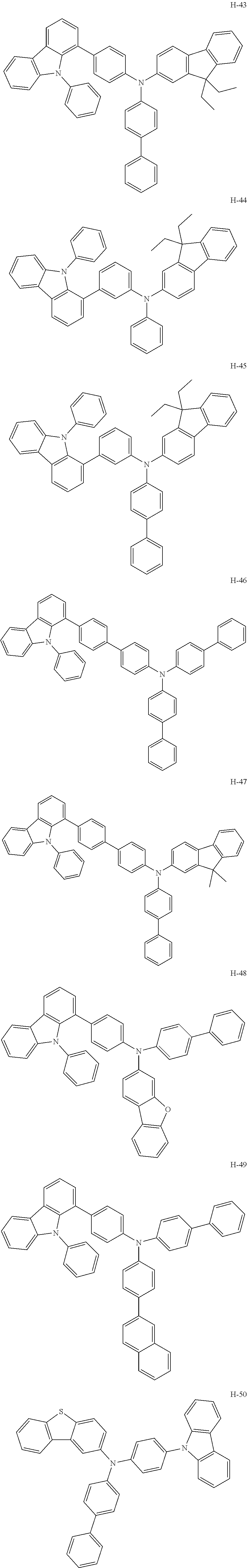 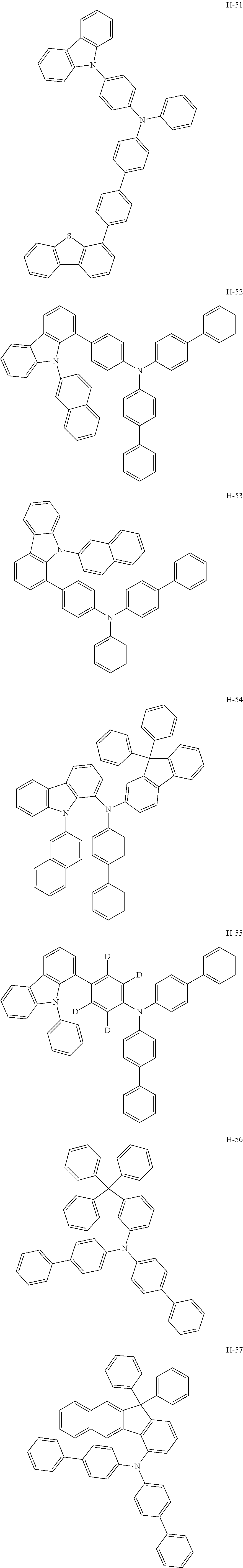 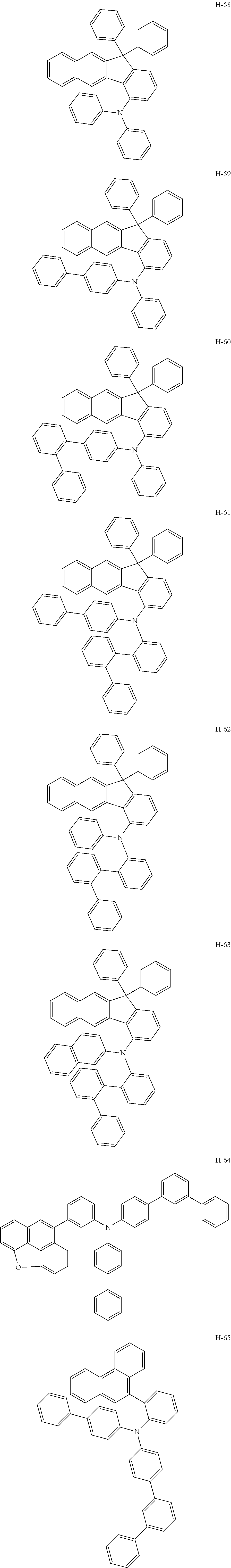 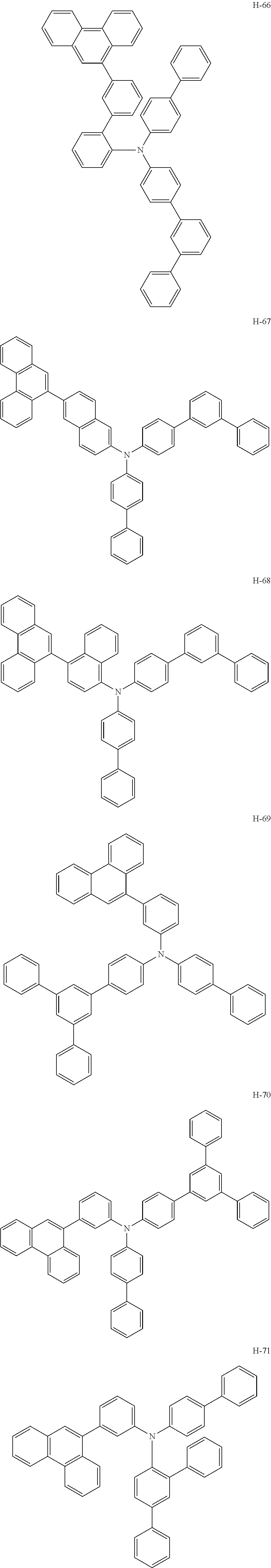 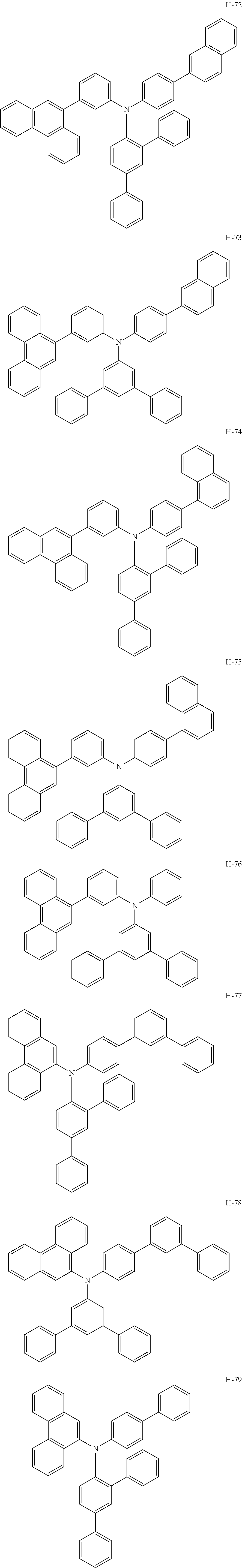 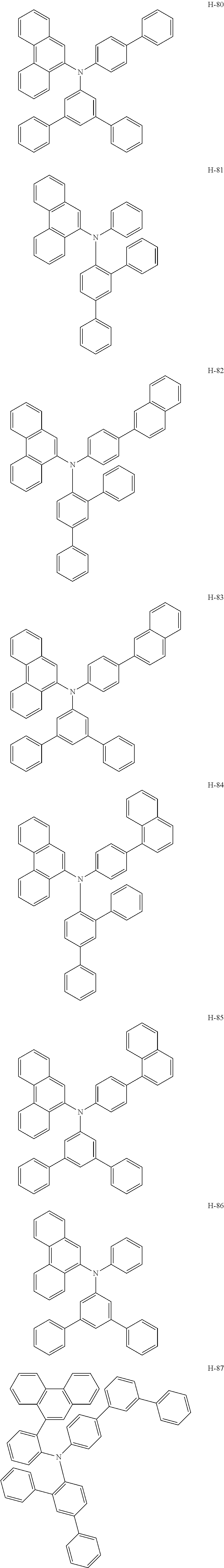 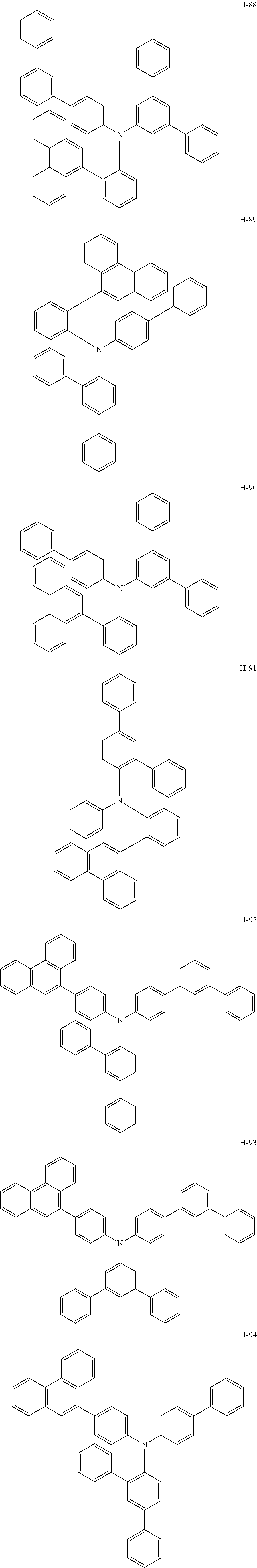 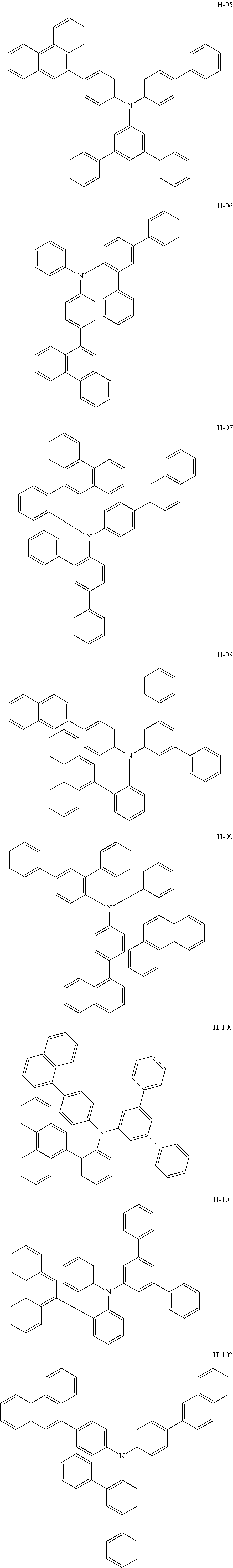 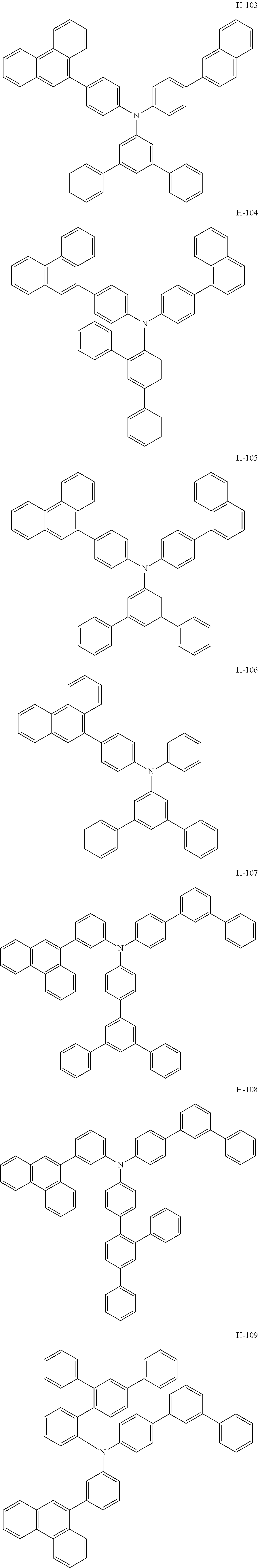 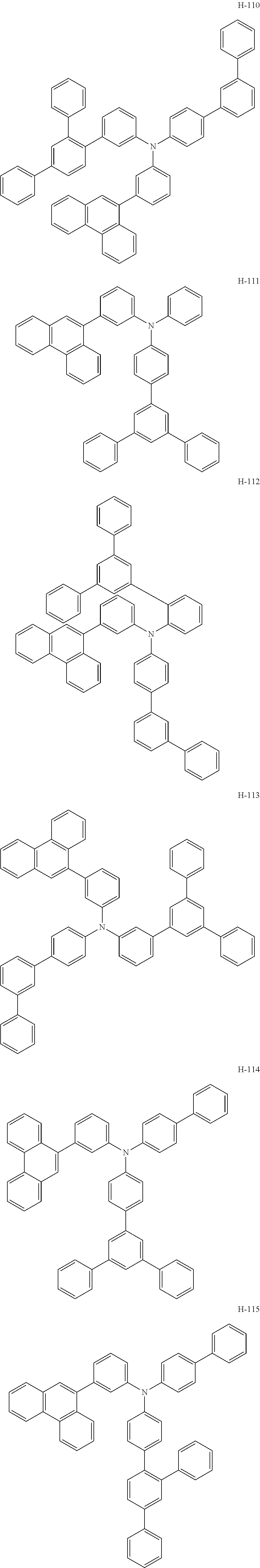 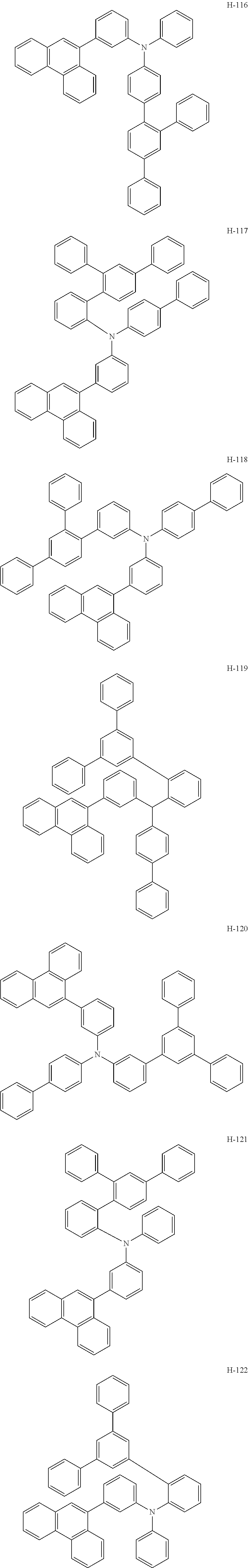 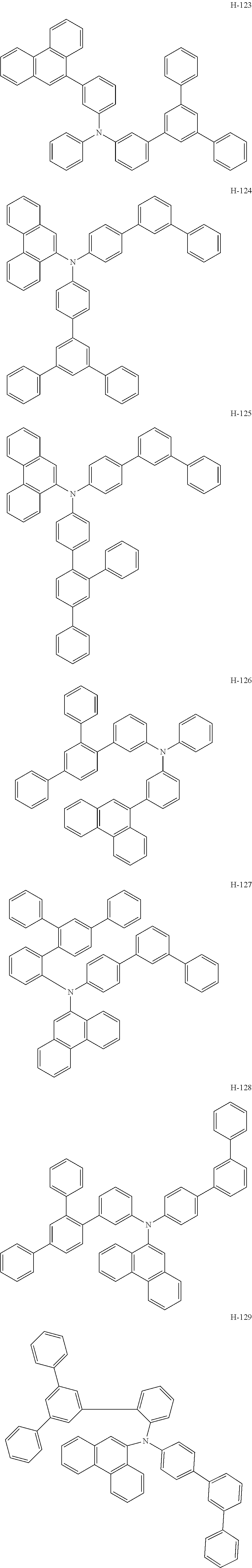 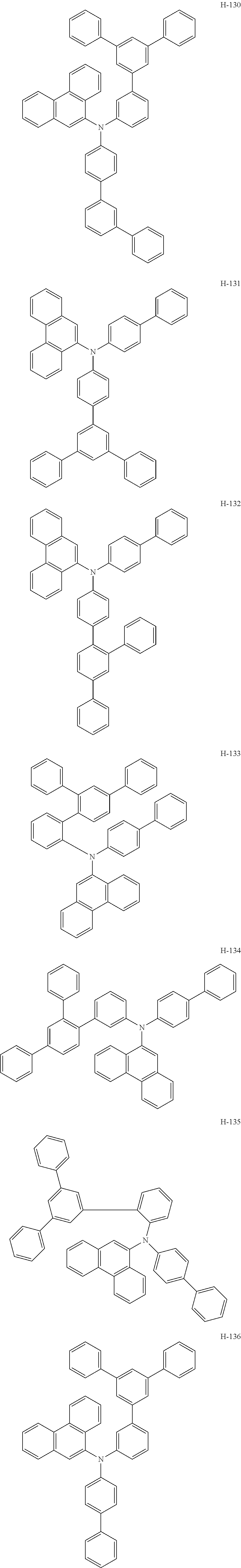 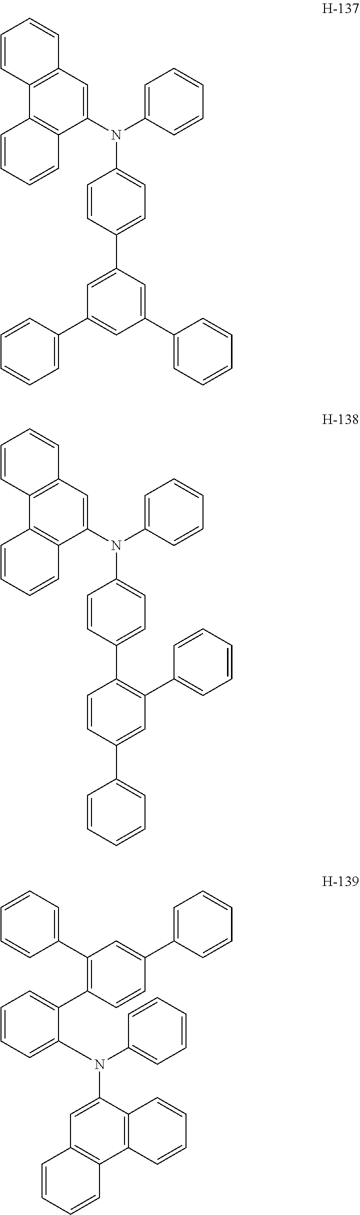 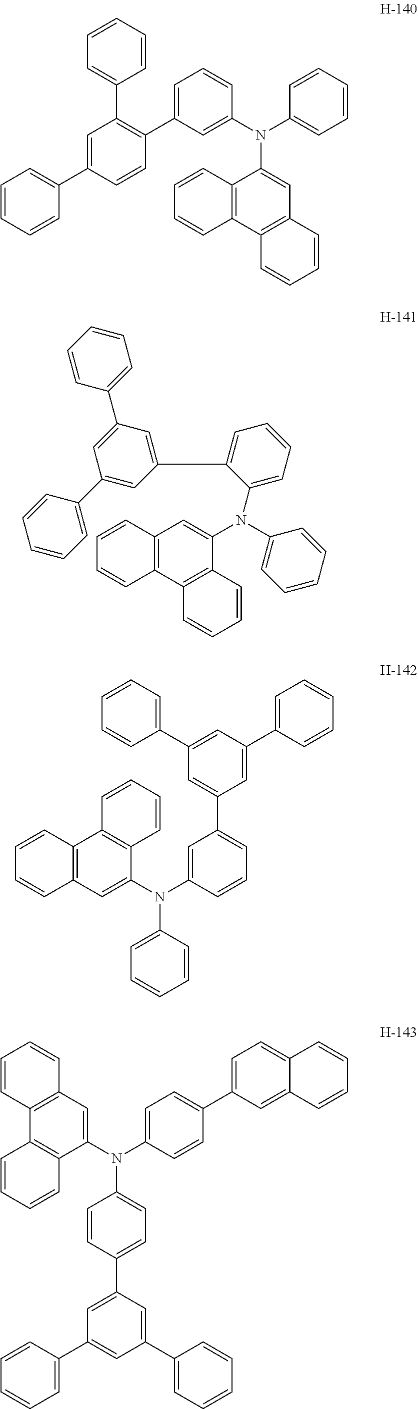 [SMILES: c1ccc(-c2ccc(-c3cccc(N(c4ccccc4)c4cc5ccccc5c5ccccc45)c3)c(-c3ccccc3)c2)cc1 ; c1ccc(-c2cc(-c3ccccc3)cc(-c3ccccc3N(c3ccccc3)c3cc4ccccc4c4ccccc34)c2)cc1 ; c1ccc(-c2cc(-c3ccccc3)cc(-c3cccc(N(c4ccccc4)c4cc5ccccc5c5ccccc45)c3)c2)cc1 ; c1ccc(-c2cc(-c3ccccc3)cc(-c3ccc(N(c4ccc(-c5ccc6ccccc6c5)cc4)c4cc5ccccc5c5ccccc45)cc3)c2)cc1] 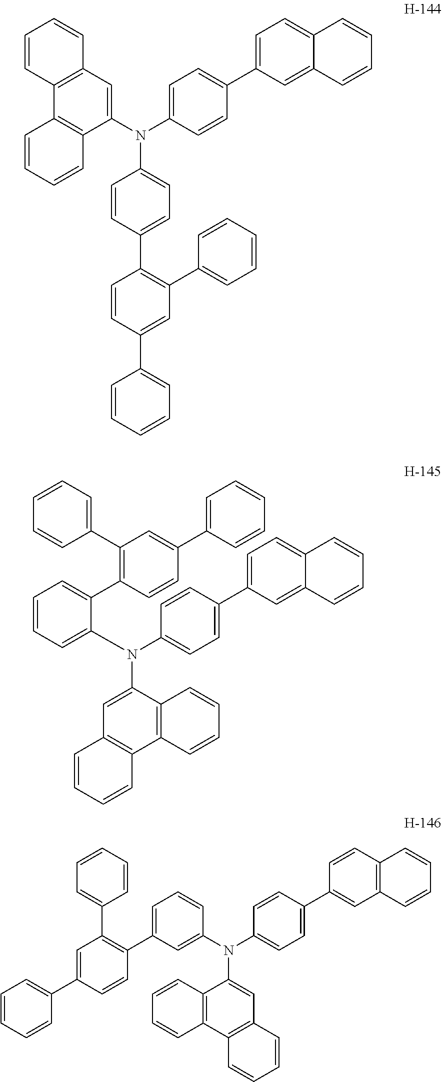 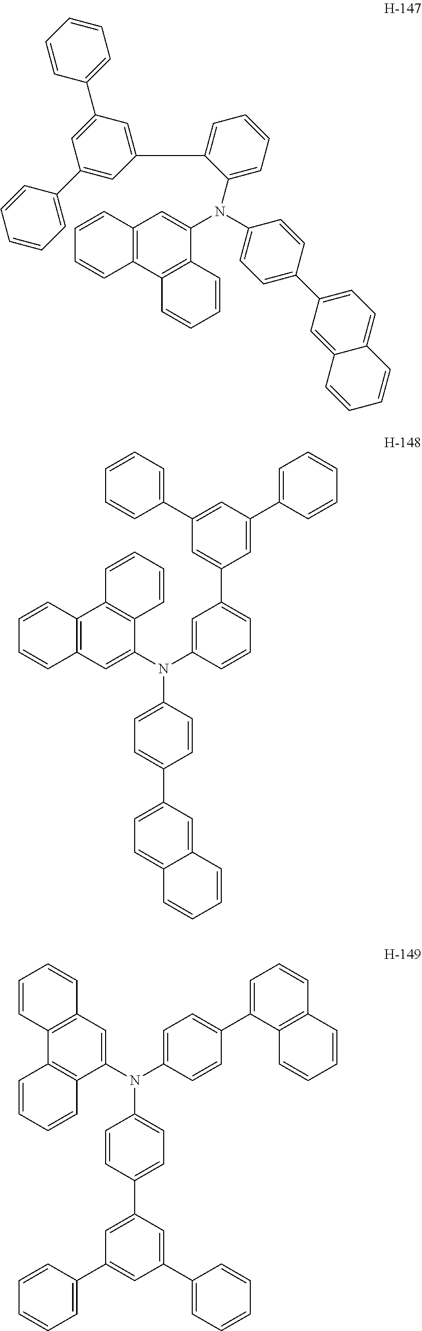 [SMILES: c1ccc(-c2cc(-c3ccccc3)cc(-c3ccccc3N(c3ccc(-c4ccc5ccccc5c4)cc3)c3cc4ccccc4c4ccccc34)c2)cc1 ; c1ccc(-c2cc(-c3ccccc3)cc(-c3cccc(N(c4ccc(-c5ccc6ccccc6c5)cc4)c4cc5ccccc5c5ccccc45)c3)c2)cc1 ; c1ccc(-c2cc(-c3ccccc3)cc(-c3ccc(N(c4ccc(-c5cccc6ccccc56)cc4)c4cc5ccccc5c5ccccc45)cc3)c2)cc1] 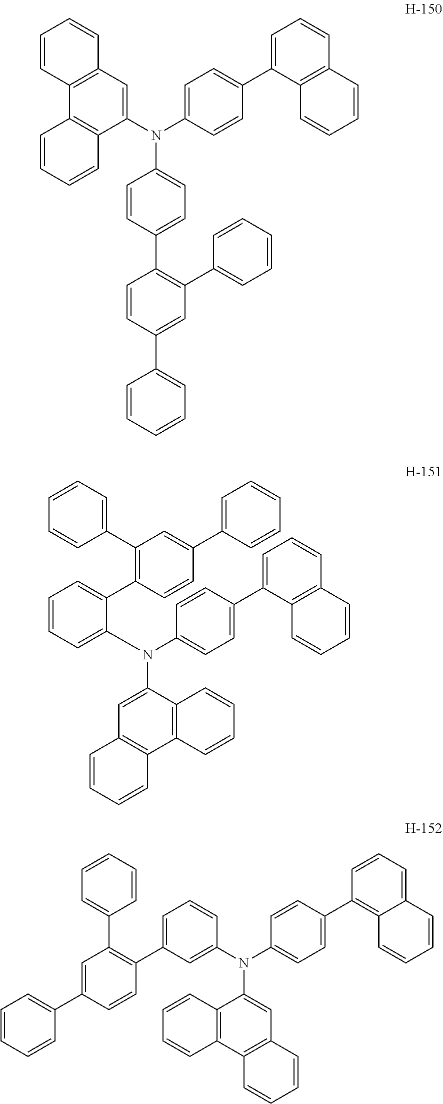 [SMILES: c1ccc(-c2ccc(-c3ccc(N(c4ccc(-c5cccc6ccccc56)cc4)c4cc5ccccc5c5ccccc45)cc3)c(-c3ccccc3)c2)cc1 ; c1ccc(-c2ccc(-c3ccccc3N(c3ccc(-c4cccc5ccccc45)cc3)c3cc4ccccc4c4ccccc34)c(-c3ccccc3)c2)cc1 ; c1ccc(-c2ccc(-c3cccc(N(c4ccc(-c5cccc6ccccc56)cc4)c4cc5ccccc5c5ccccc45)c3)c(-c3ccccc3)c2)cc1] 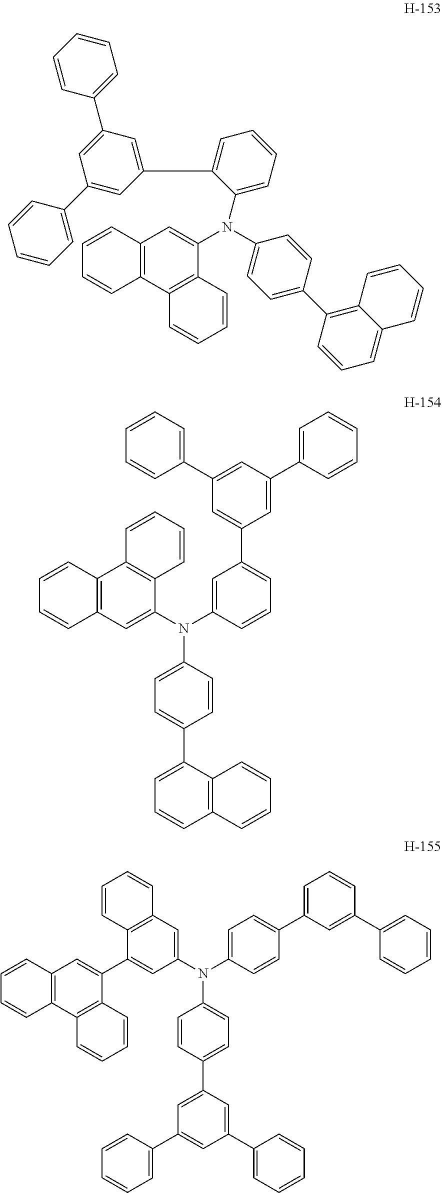 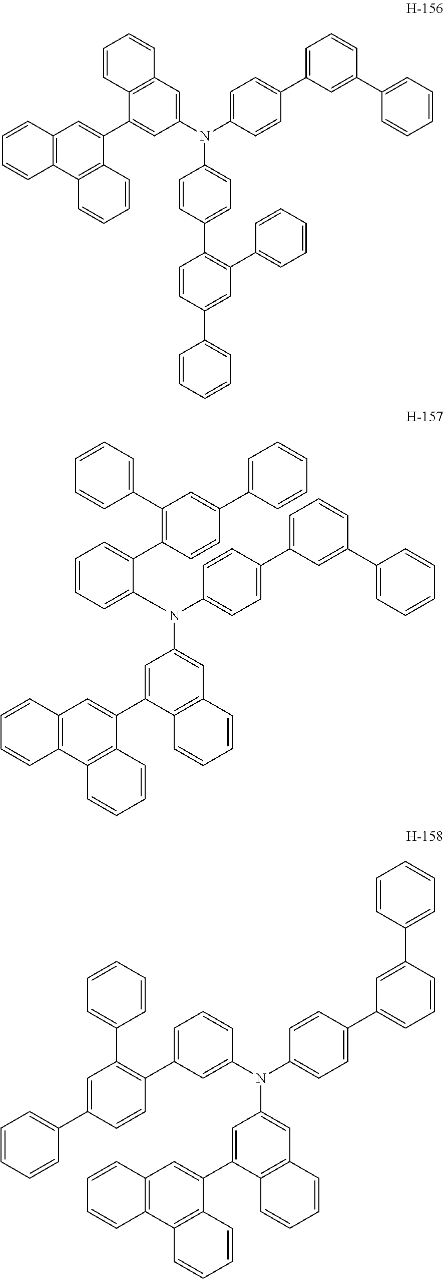 [SMILES: c1ccc(-c2cccc(-c3ccc(N(c4ccc(-c5ccc(-c6ccccc6)cc5-c5ccccc5)cc4)c4cc(-c5cc6ccccc6c6ccccc56)c5ccccc5c4)cc3)c2)cc1 ; c1ccc(-c2cccc(-c3ccc(N(c4cc(-c5cc6ccccc6c6ccccc56)c5ccccc5c4)c4ccccc4-c4ccc(-c5ccccc5)cc4-c4ccccc4)cc3)c2)cc1 ; c1ccc(-c2cccc(-c3ccc(N(c4cccc(-c5ccc(-c6ccccc6)cc5-c5ccccc5)c4)c4cc(-c5cc6ccccc6c6ccccc56)c5ccccc5c4)cc3)c2)cc1] 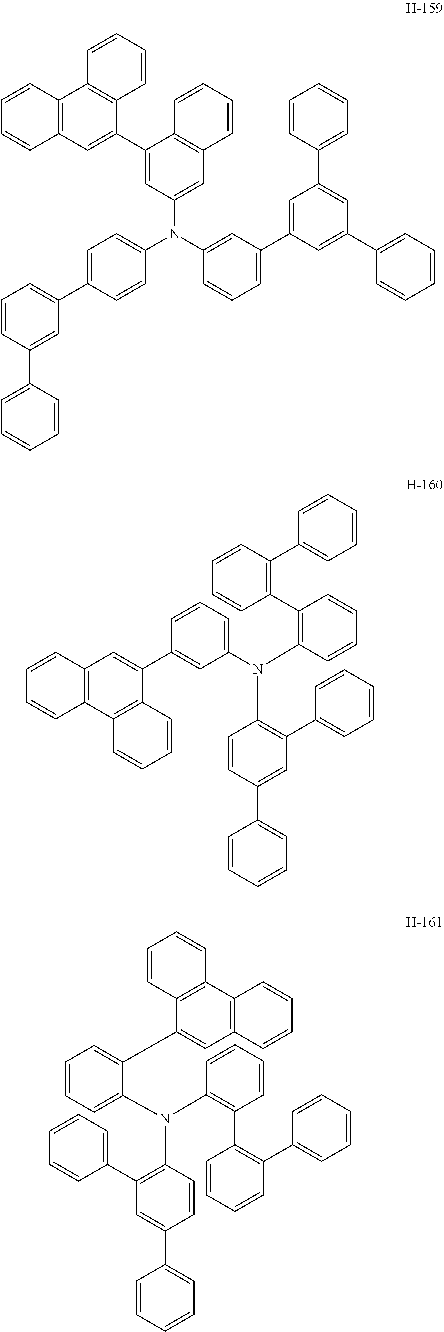 [SMILES: c1ccc(-c2cccc(-c3ccc(N(c4cccc(-c5cc(-c6ccccc6)cc(-c6ccccc6)c5)c4)c4cc(-c5cc6ccccc6c6ccccc56)c5ccccc5c4)cc3)c2)cc1 ; c1ccc(-c2ccc(N(c3cccc(-c4cc5ccccc5c5ccccc45)c3)c3ccccc3-c3ccccc3-c3ccccc3)c(-c3ccccc3)c2)cc1 ; c1ccc(-c2ccc(N(c3ccccc3-c3ccccc3-c3ccccc3)c3ccccc3-c3cc4ccccc4c4ccccc34)c(-c3ccccc3)c2)cc1] 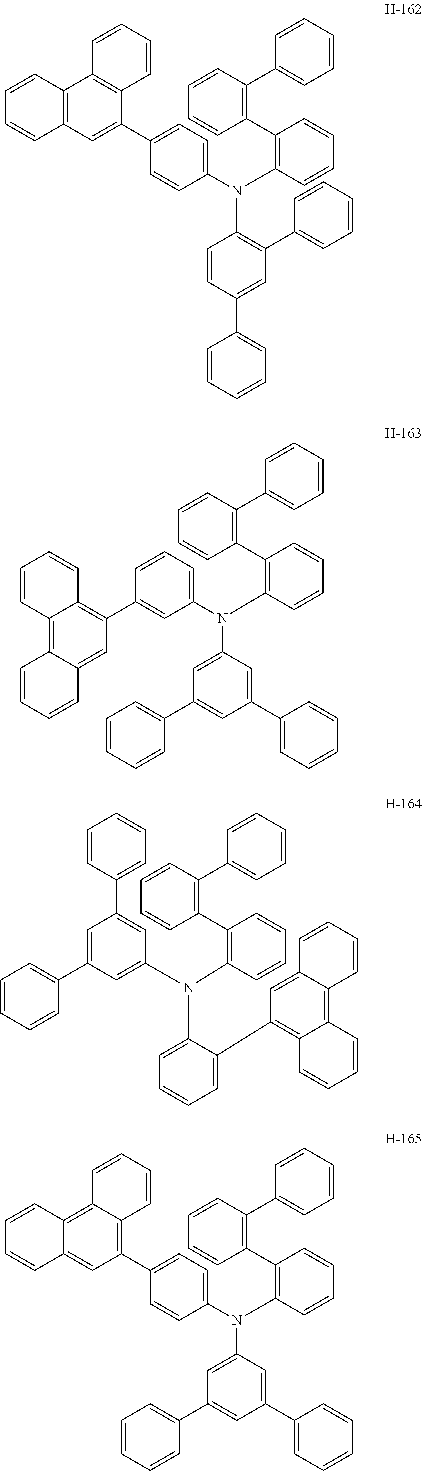 [SMILES: c1ccc(-c2ccc(N(c3ccc(-c4cc5ccccc5c5ccccc45)cc3)c3ccccc3-c3ccccc3-c3ccccc3)c(-c3ccccc3)c2)cc1 ; c1ccc(-c2cc(-c3ccccc3)cc(N(c3cccc(-c4cc5ccccc5c5ccccc45)c3)c3ccccc3-c3ccccc3-c3ccccc3)c2)cc1 ; c1ccc(-c2cc(-c3ccccc3)cc(N(c3ccccc3-c3ccccc3-c3ccccc3)c3ccccc3-c3cc4ccccc4c4ccccc34)c2)cc1 ; c1ccc(-c2cc(-c3ccccc3)cc(N(c3ccc(-c4cc5ccccc5c5ccccc45)cc3)c3ccccc3-c3ccccc3-c3ccccc3)c2)cc1] 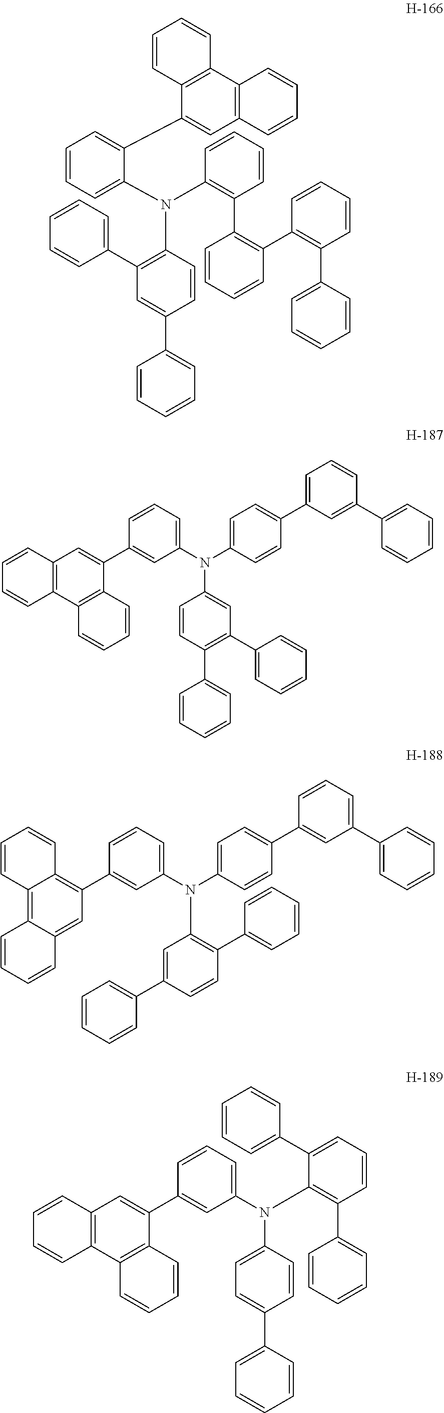 [SMILES: c1ccc(-c2ccc(N(c3ccccc3-c3ccccc3-c3ccccc3-c3ccccc3)c3ccccc3-c3cc4ccccc4c4ccccc34)c(-c3ccccc3)c2)cc1 ; c1ccc(-c2cccc(-c3ccc(N(c4cccc(-c5cc6ccccc6c6ccccc56)c4)c4ccc(-c5ccccc5)c(-c5ccccc5)c4)cc3)c2)cc1 ; c1ccc(-c2cccc(-c3ccc(N(c4cccc(-c5cc6ccccc6c6ccccc56)c4)c4cc(-c5ccccc5)ccc4-c4ccccc4)cc3)c2)cc1 ; c1ccc(-c2ccc(N(c3cccc(-c4cc5ccccc5c5ccccc45)c3)c3c(-c4ccccc4)cccc3-c3ccccc3)cc2)cc1] 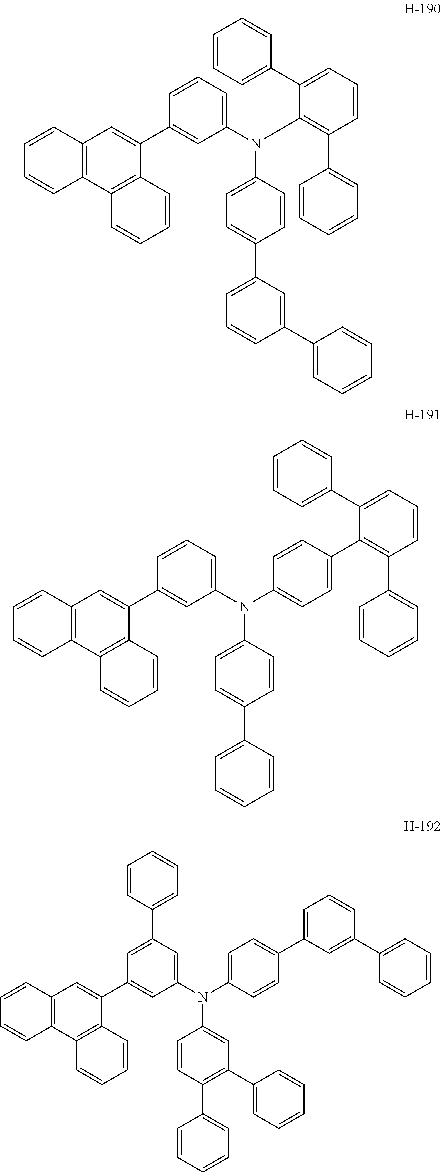 [SMILES: c1ccc(-c2cccc(-c3ccc(N(c4cccc(-c5cc6ccccc6c6ccccc56)c4)c4c(-c5ccccc5)cccc4-c4ccccc4)cc3)c2)cc1 ; c1ccc(-c2ccc(N(c3ccc(-c4c(-c5ccccc5)cccc4-c4ccccc4)cc3)c3cccc(-c4cc5ccccc5c5ccccc45)c3)cc2)cc1 ; c1ccc(-c2cccc(-c3ccc(N(c4cc(-c5ccccc5)cc(-c5cc6ccccc6c6ccccc56)c4)c4ccc(-c5ccccc5)c(-c5ccccc5)c4)cc3)c2)cc1] 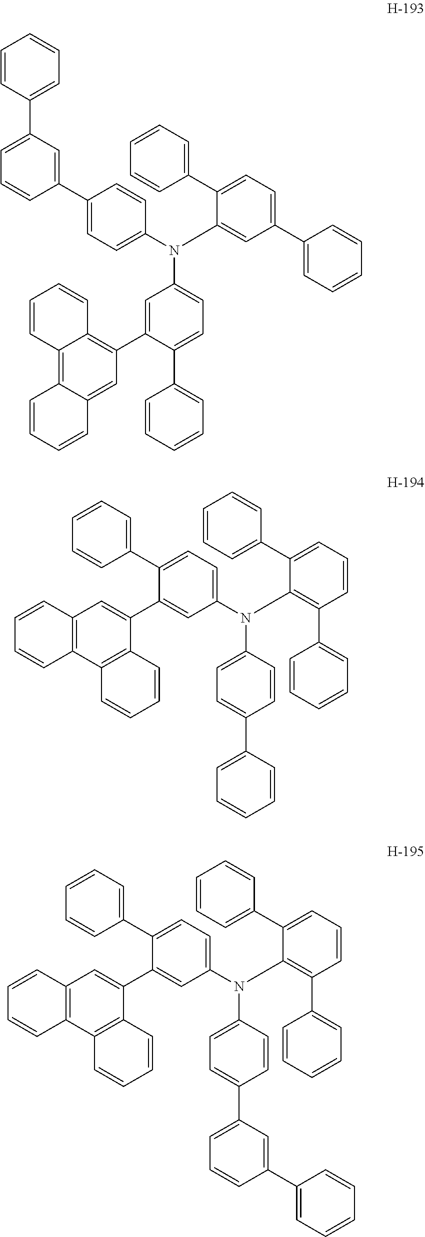 [SMILES: c1ccc(-c2cccc(-c3ccc(N(c4ccc(-c5ccccc5)c(-c5cc6ccccc6c6ccccc56)c4)c4cc(-c5ccccc5)ccc4-c4ccccc4)cc3)c2)cc1 ; c1ccc(-c2ccc(N(c3ccc(-c4ccccc4)c(-c4cc5ccccc5c5ccccc45)c3)c3c(-c4ccccc4)cccc3-c3ccccc3)cc2)cc1 ; c1ccc(-c2cccc(-c3ccc(N(c4ccc(-c5ccccc5)c(-c5cc6ccccc6c6ccccc56)c4)c4c(-c5ccccc5)cccc4-c4ccccc4)cc3)c2)cc1] 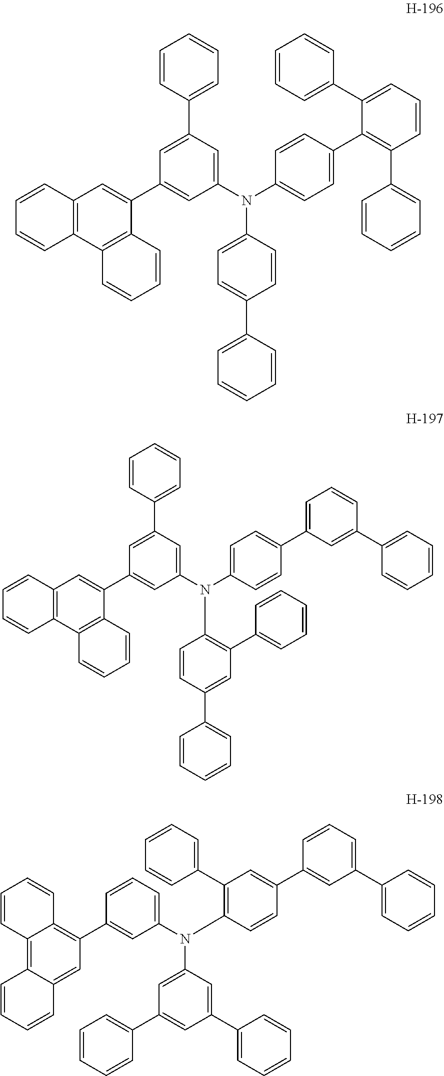 [SMILES: c1ccc(-c2ccc(N(c3ccc(-c4c(-c5ccccc5)cccc4-c4ccccc4)cc3)c3cc(-c4ccccc4)cc(-c4cc5ccccc5c5ccccc45)c3)cc2)cc1 ; c1ccc(-c2cccc(-c3ccc(N(c4cc(-c5ccccc5)cc(-c5cc6ccccc6c6ccccc56)c4)c4ccc(-c5ccccc5)cc4-c4ccccc4)cc3)c2)cc1 ; c1ccc(-c2cccc(-c3ccc(N(c4cc(-c5ccccc5)cc(-c5ccccc5)c4)c4cccc(-c5cc6ccccc6c6ccccc56)c4)c(-c4ccccc4)c3)c2)cc1] 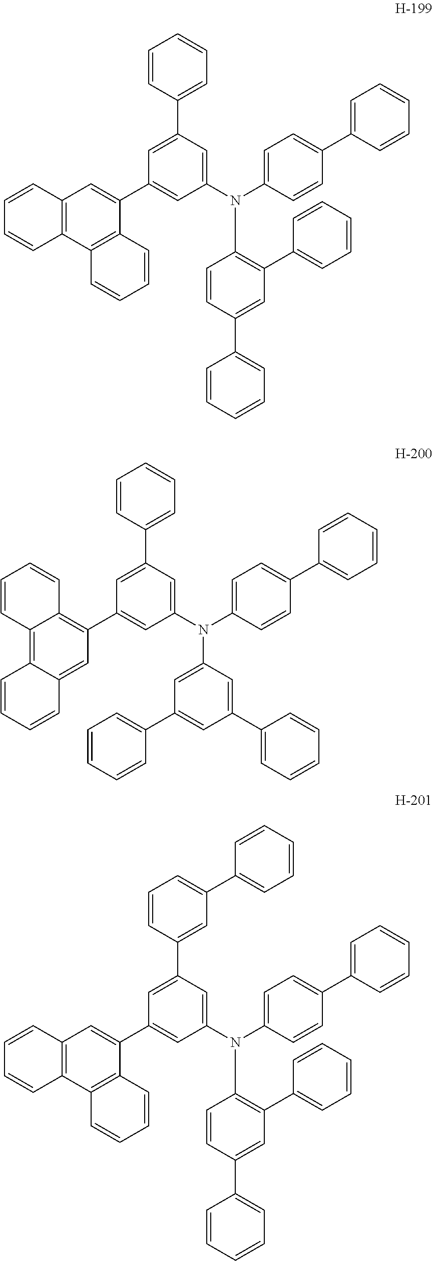 [SMILES: c1ccc(-c2ccc(N(c3cc(-c4ccccc4)cc(-c4cc5ccccc5c5ccccc45)c3)c3ccc(-c4ccccc4)cc3-c3ccccc3)cc2)cc1 ; c1ccc(-c2ccc(N(c3cc(-c4ccccc4)cc(-c4ccccc4)c3)c3cc(-c4ccccc4)cc(-c4cc5ccccc5c5ccccc45)c3)cc2)cc1 ; c1ccc(-c2ccc(N(c3cc(-c4cccc(-c5ccccc5)c4)cc(-c4cc5ccccc5c5ccccc45)c3)c3ccc(-c4ccccc4)cc3-c3ccccc3)cc2)cc1] 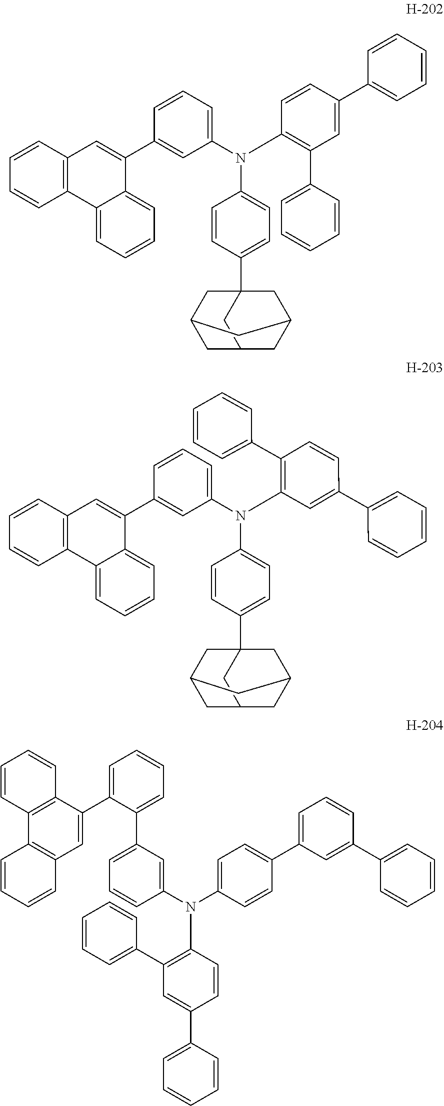 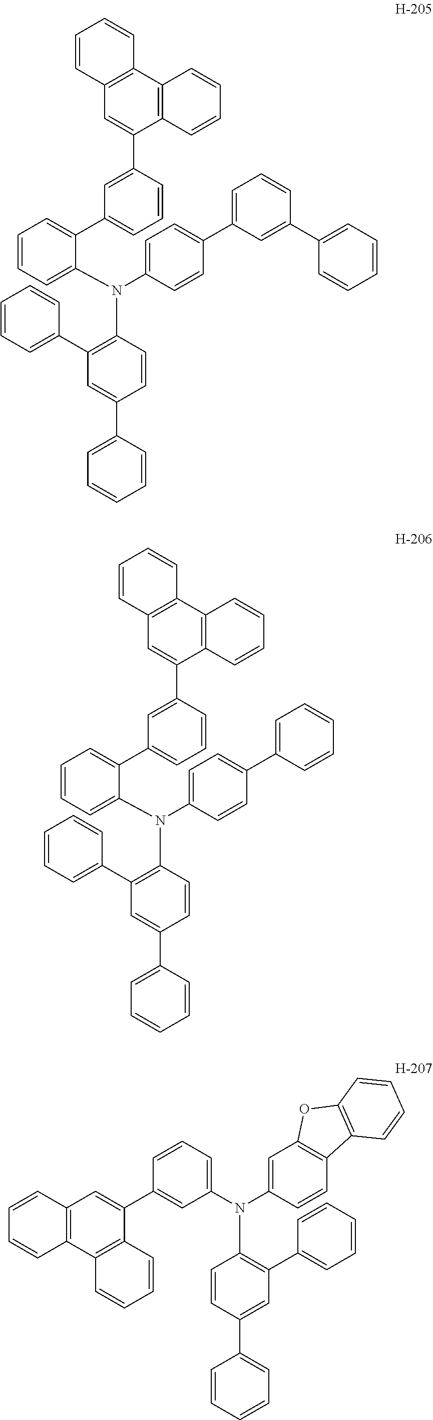 [SMILES: c1ccc(-c2cccc(-c3ccc(N(c4ccccc4-c4cccc(-c5cc6ccccc6c6ccccc56)c4)c4ccc(-c5ccccc5)cc4-c4ccccc4)cc3)c2)cc1 ; c1ccc(-c2ccc(N(c3ccccc3-c3cccc(-c4cc5ccccc5c5ccccc45)c3)c3ccc(-c4ccccc4)cc3-c3ccccc3)cc2)cc1 ; c1ccc(-c2ccc(N(c3cccc(-c4cc5ccccc5c5ccccc45)c3)c3ccc4c(c3)oc3ccccc34)c(-c3ccccc3)c2)cc1] 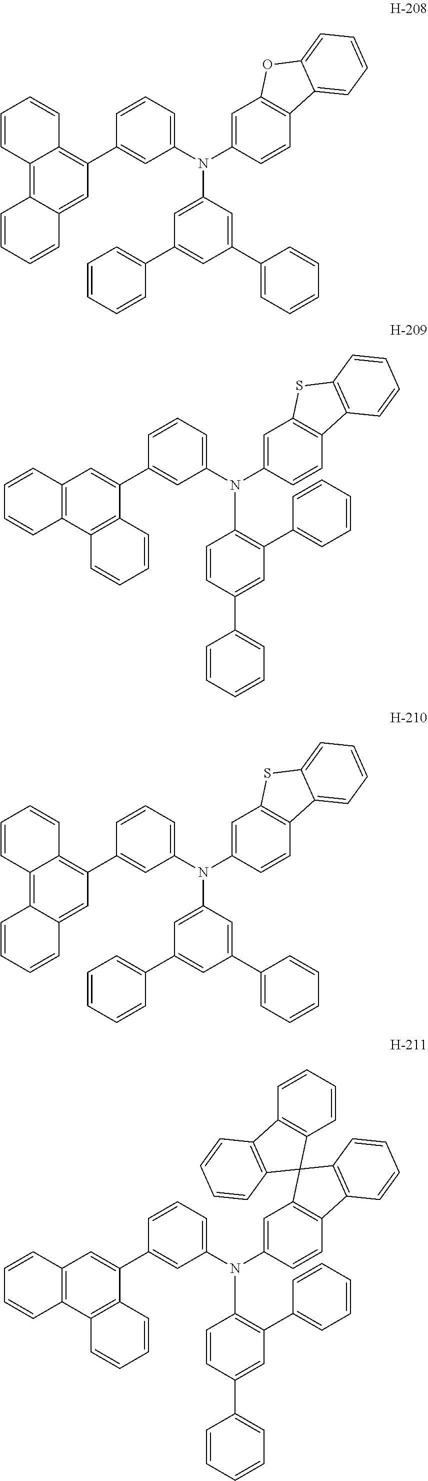 [SMILES: c1ccc(-c2cc(-c3ccccc3)cc(N(c3cccc(-c4cc5ccccc5c5ccccc45)c3)c3ccc4c(c3)oc3ccccc34)c2)cc1 ; c1ccc(-c2ccc(N(c3cccc(-c4cc5ccccc5c5ccccc45)c3)c3ccc4c(c3)sc3ccccc34)c(-c3ccccc3)c2)cc1 ; c1ccc(-c2cc(-c3ccccc3)cc(N(c3cccc(-c4cc5ccccc5c5ccccc45)c3)c3ccc4c(c3)sc3ccccc34)c2)cc1 ; c1ccc(-c2ccc(N(c3cccc(-c4cc5ccccc5c5ccccc45)c3)c3ccc4c(c3)C3(c5ccccc5-c5ccccc53)c3ccccc3-4)c(-c3ccccc3)c2)cc1] 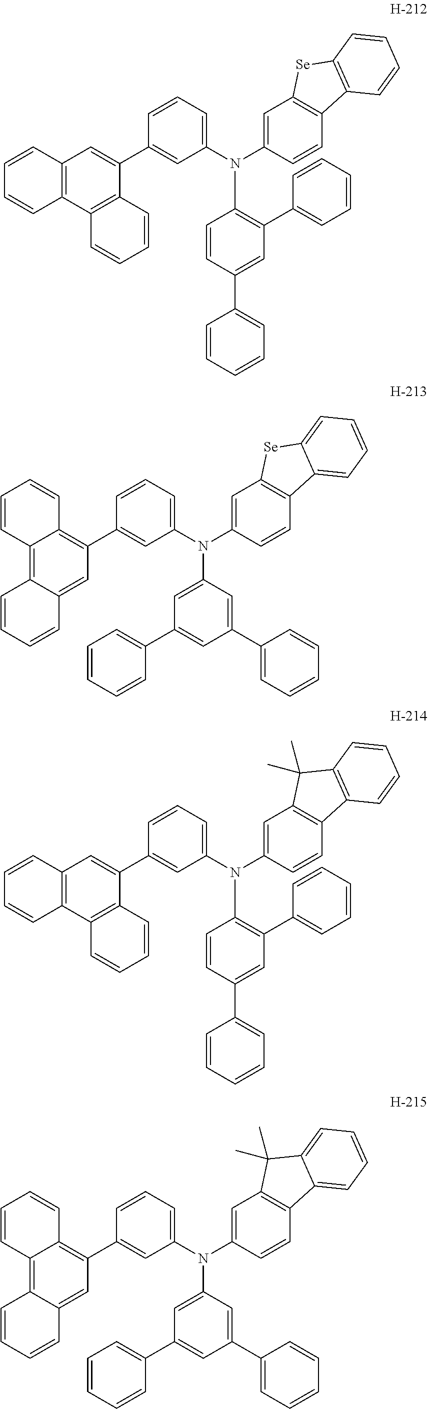 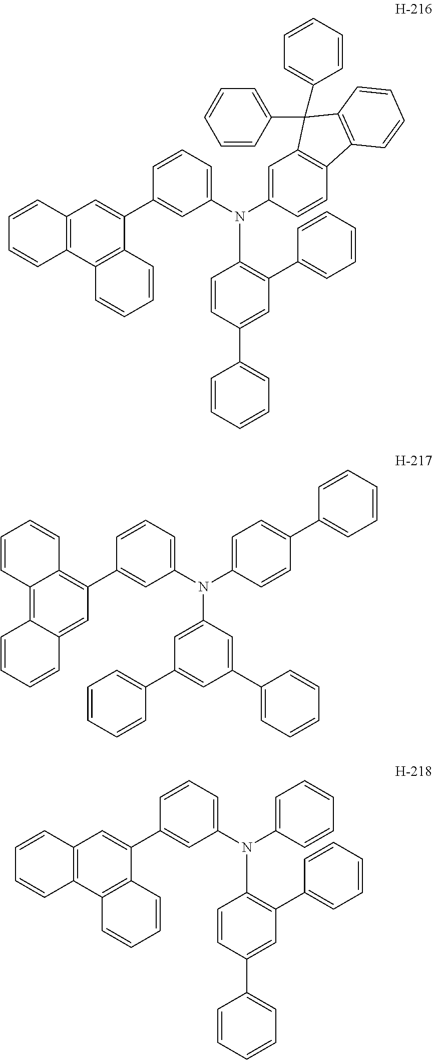 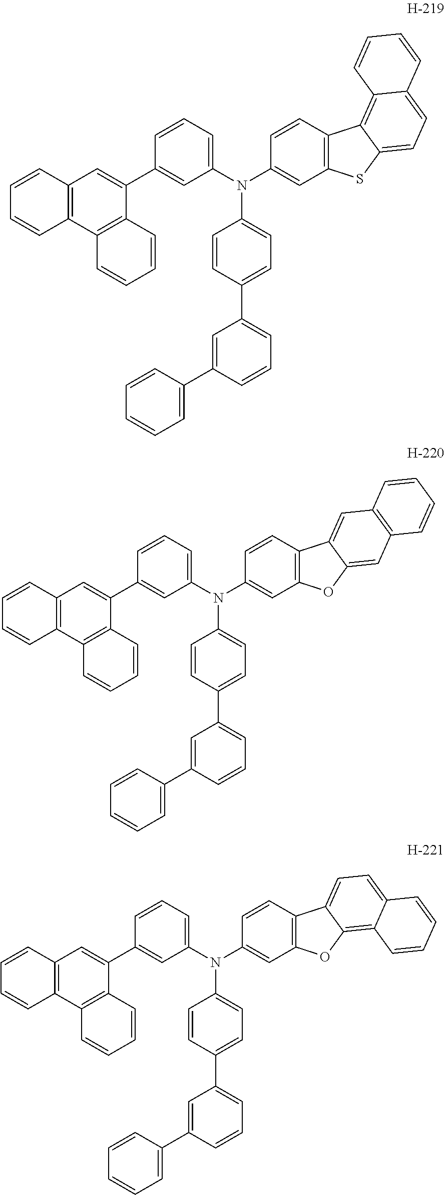 [SMILES: c1ccc(-c2cccc(-c3ccc(N(c4cccc(-c5cc6ccccc6c6ccccc56)c4)c4ccc5c(c4)sc4ccc6ccccc6c45)cc3)c2)cc1 ; c1ccc(-c2cccc(-c3ccc(N(c4cccc(-c5cc6ccccc6c6ccccc56)c4)c4ccc5c(c4)oc4cc6ccccc6cc45)cc3)c2)cc1 ; c1ccc(-c2cccc(-c3ccc(N(c4cccc(-c5cc6ccccc6c6ccccc56)c4)c4ccc5c(c4)oc4c6ccccc6ccc54)cc3)c2)cc1] 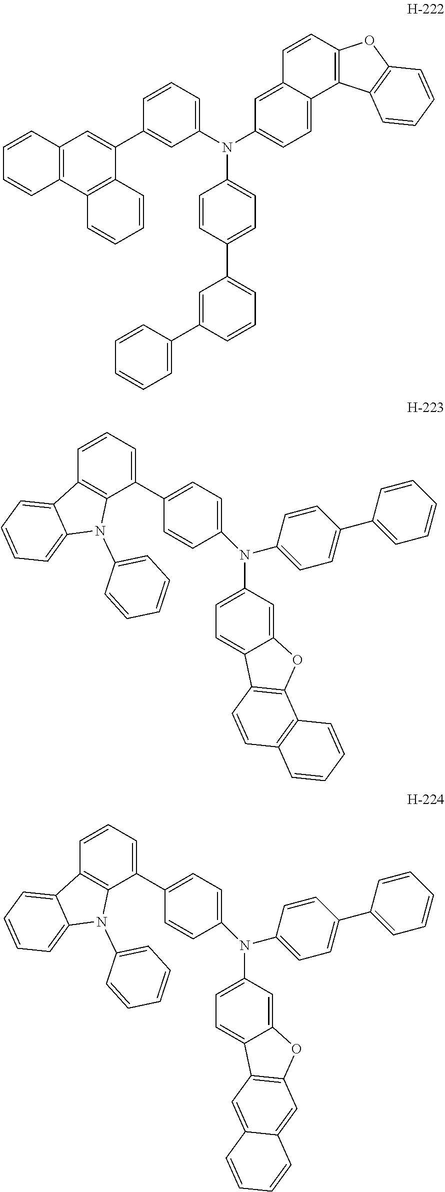 [SMILES: c1ccc(-c2cccc(-c3ccc(N(c4cccc(-c5cc6ccccc6c6ccccc56)c4)c4ccc5c(ccc6oc7ccccc7c65)c4)cc3)c2)cc1 ; c1ccc(-c2ccc(N(c3ccc(-c4cccc5c6ccccc6n(-c6ccccc6)c45)cc3)c3ccc4c(c3)oc3c5ccccc5ccc43)cc2)cc1 ; c1ccc(-c2ccc(N(c3ccc(-c4cccc5c6ccccc6n(-c6ccccc6)c45)cc3)c3ccc4c(c3)oc3cc5ccccc5cc34)cc2)cc1] 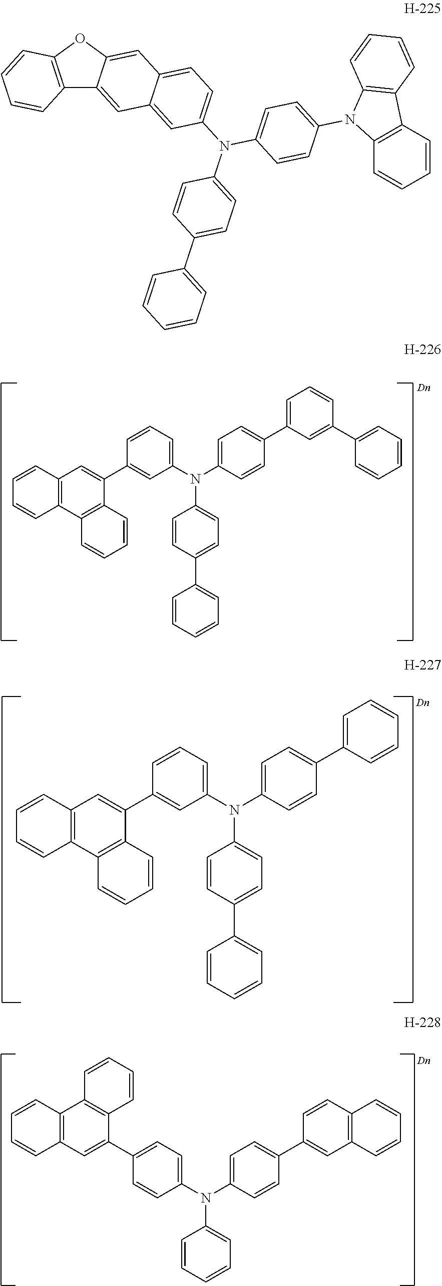 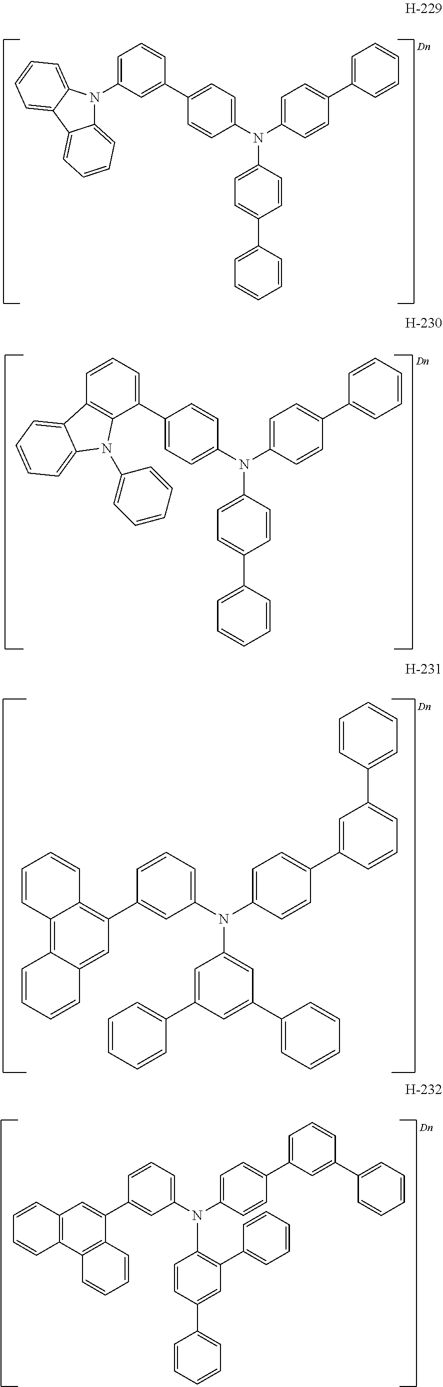 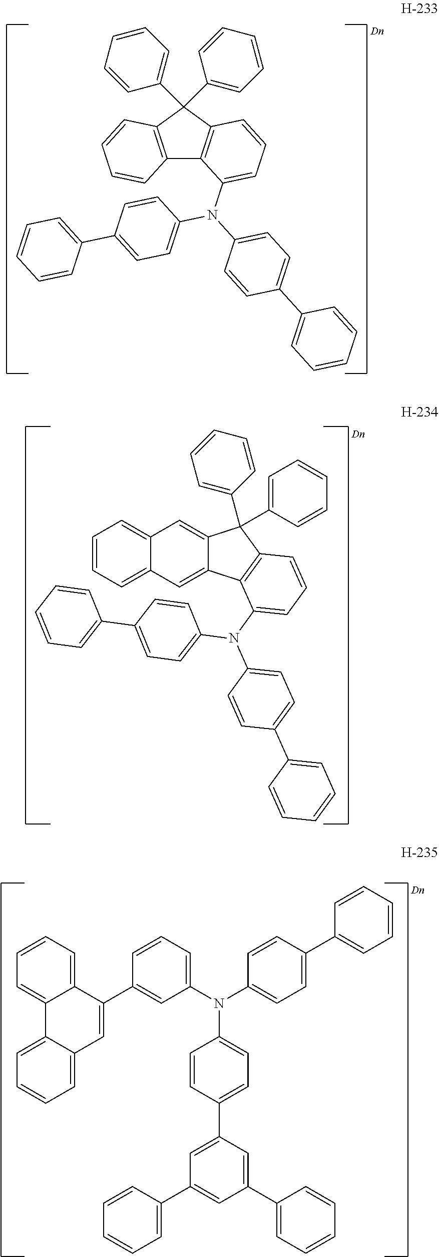
wherein in the compounds above, Dn represents that n number of hydrogens are replaced with deuterium, and n represents an integer of 1 or more, which is an integer of 1 to the maximum number of hydrogens in the compound.

11. The organic electroluminescent device according to claim 1, wherein the light-emitting layer comprises a light-emitting layer that emits red light.

12. An organic electroluminescent compound represented by the following Formula 1: 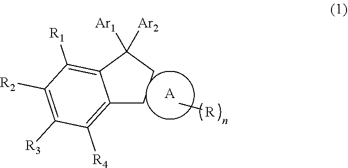
wherein in Formula 1,
ring A represents a substituted or unsubstituted benzene, a substituted or unsubstituted naphthalene, or a substituted or unsubstituted phenanthrene;
Ar₁ and Ar₂ each independently represent a substituted or unsubstituted (C2-C30)alkyl;
R and R₁ to R₄ each independently represent hydrogen, deuterium, a halogen, a cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, or a substituted or unsubstituted (C1-C30)alkoxy, or may be linked to an adjacent substituent(s) to form a ring(s); with a proviso that at least one of R and R₁ to R₄ is represented by -L₁-NAr₃Ar₄;
L₁ represents a single bond, a substituted or unsubstituted (C6-C30)arylene, or a substituted or unsubstituted (3- to 30-membered)heteroarylene;
Ar₃ and Ar₄ each independently represent a substituted or unsubstituted (C6-C30)aryl, or a substituted or unsubstituted (3- to 30-membered)heteroaryl; and
n represents an integer of 1 to 8, if n is plural, each of n may be the same as or different from each other.

13. The organic electroluminescent compound according to claim 12, wherein Formula 1 is represented by any one of the following Formulas 1-1 to 1-11: 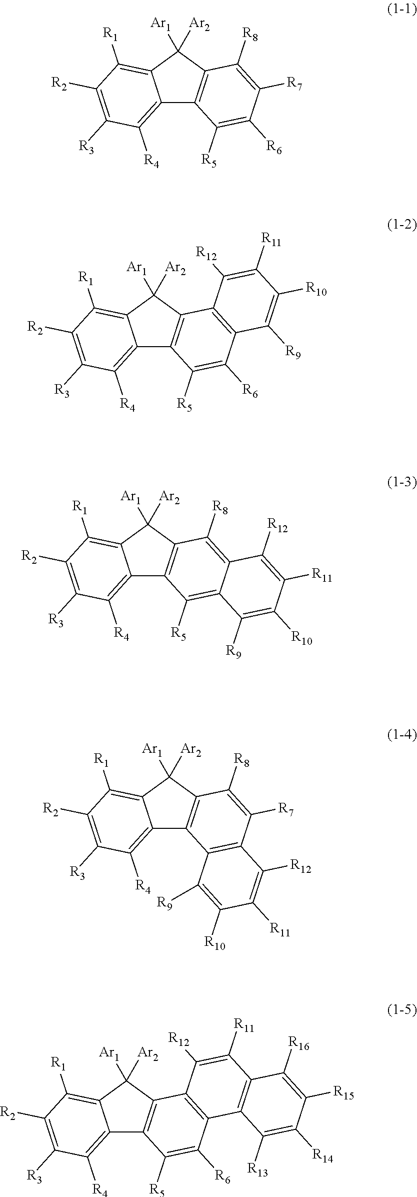 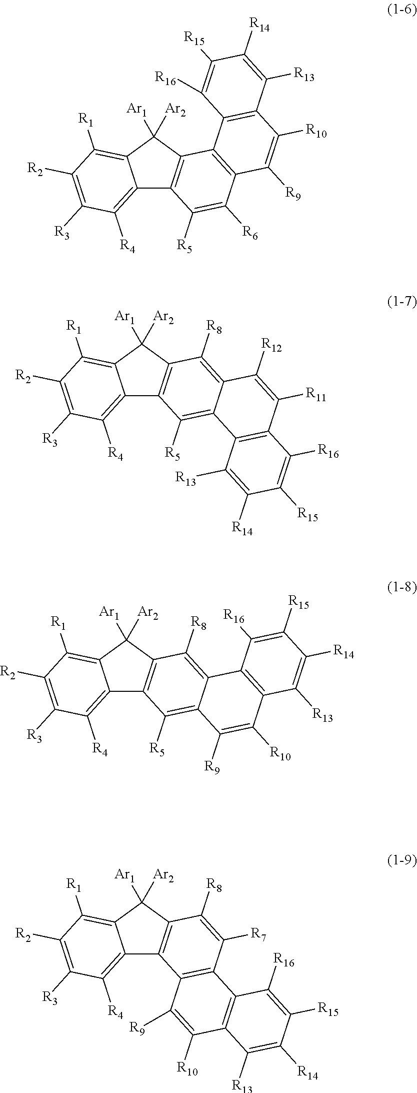 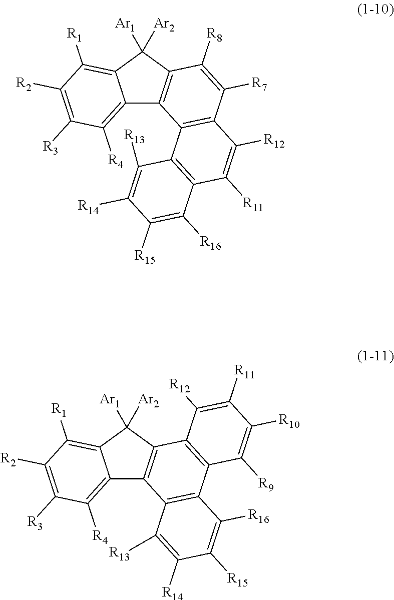
wherein in Formulas 1-1 to 1-11,
Ar₁ and Ar₂ each independently represent a substituted or unsubstituted (C2-C30)alkyl;
R₁ to R₁₆ each independently represent hydrogen, deuterium, a halogen, a cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, or a substituted or unsubstituted (C1-C30)alkoxy, or may be linked to an adjacent substituent(s) to form a ring(s); with a proviso that at least one of R₁ to R₁₆ is represented by -L₁-NAr₃Ar₄;
L₁ represents a single bond, a substituted or unsubstituted (C6-C30)arylene, or a substituted or unsubstituted (3- to 30-membered)heteroarylene; and
Ar₃ and Ar₄ each independently represent a substituted or unsubstituted (C6-C30)aryl, or a substituted or unsubstituted (3- to 30-membered)heteroaryl.

14. The organic electroluminescent compound according to claim 12, wherein Ar₃ in Formula 1 is represented by any one of the following Formulas: 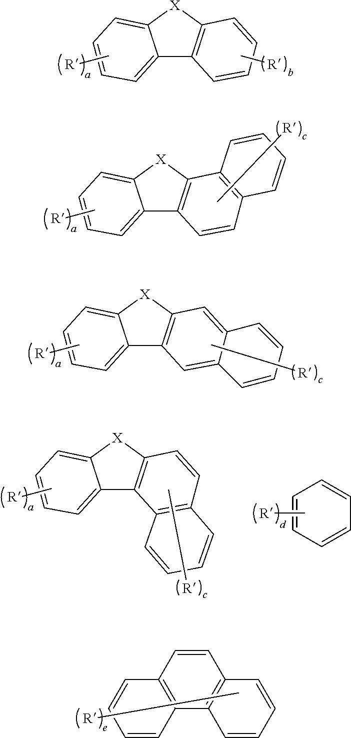
wherein in the above Formulas,
X represents O, S, NR₇₃, or CR₇₄R₇₅;
R′ and R₇₃ to R₇₅ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di(C1-C30)alkylamino, a substituted or unsubstituted mono- or di(C6-C30)arylamino, or a substituted or unsubstituted (C1-C30)alkyl(C6-C30)arylamino, or may be linked to an adjacent substituent(s) to form a substituted or unsubstituted (3- to 30-membered) monocyclic or polycyclic ring(s);
wherein in each Formula, any one of R′ is linked to L₁ of Formula 1; and
a and b each independently represent an integer of 1 to 4, c and d each independently represent an integer of 1 to 6, e represents an integer of 1 to 10, and if a to e represent an integer of 2 or more, each of R′ may be the same as or different from each other.

15. The organic electroluminescent compound according to claim 12, wherein the compound represented by Formula 1 is at least one selected from the following compounds: 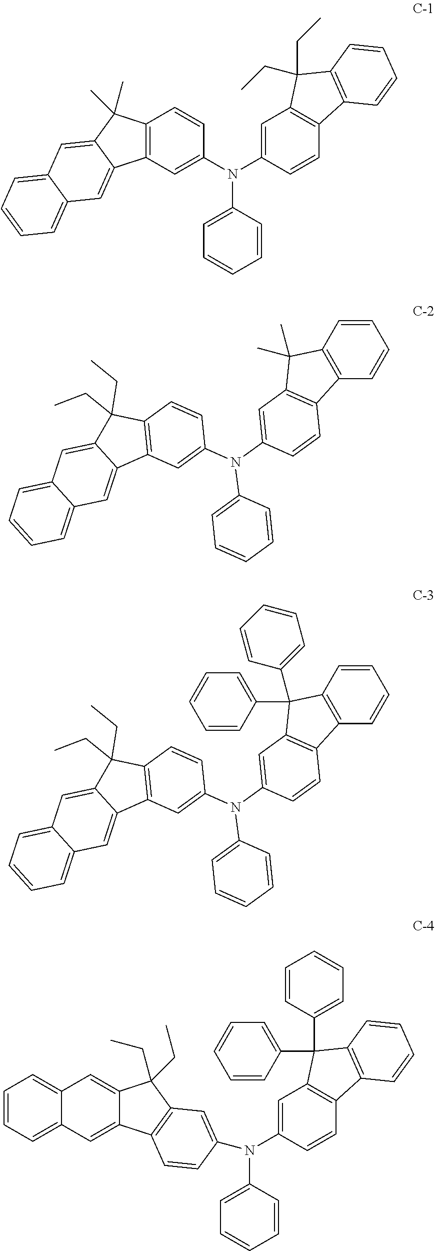 [SMILES: CCC1(CC)c2ccccc2-c2ccc(N(c3ccccc3)c3ccc4c(c3)-c3cc5ccccc5cc3C4(C)C)cc21 ; CCC1(CC)c2ccc(N(c3ccccc3)c3ccc4c(c3)C(C)(C)c3ccccc3-4)cc2-c2cc3ccccc3cc21 ; CCC1(CC)c2ccc(N(c3ccccc3)c3ccc4c(c3)C(c3ccccc3)(c3ccccc3)c3ccccc3-4)cc2-c2cc3ccccc3cc21 ; CCC1(CC)c2cc(N(c3ccccc3)c3ccc4c(c3)C(c3ccccc3)(c3ccccc3)c3ccccc3-4)ccc2-c2cc3ccccc3cc21] 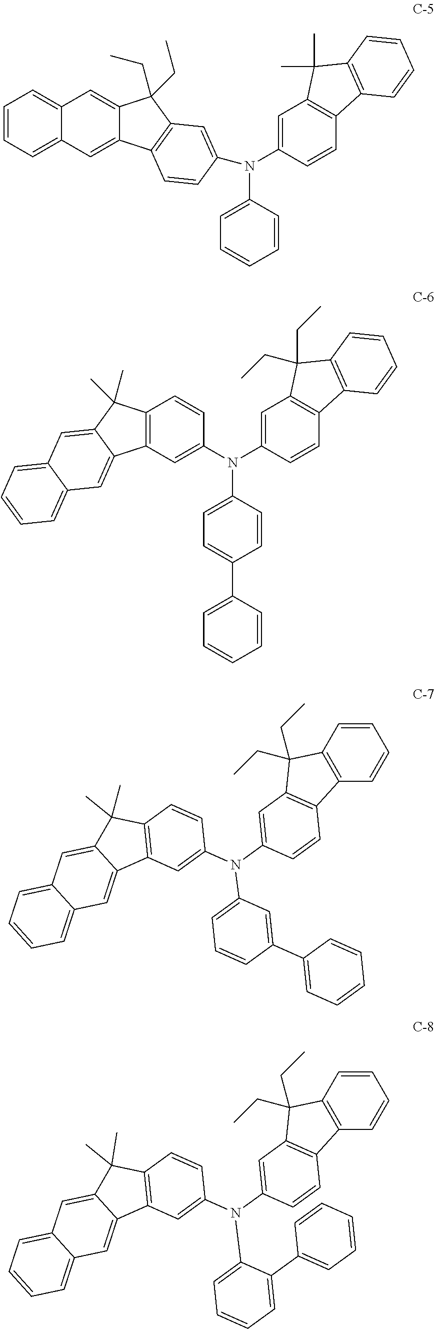 [SMILES: CCC1(CC)c2cc(N(c3ccccc3)c3ccc4c(c3)C(C)(C)c3ccccc3-4)ccc2-c2cc3ccccc3cc21 ; CCC1(CC)c2ccccc2-c2ccc(N(c3ccc(-c4ccccc4)cc3)c3ccc4c(c3)-c3cc5ccccc5cc3C4(C)C)cc21 ; CCC1(CC)c2ccccc2-c2ccc(N(c3cccc(-c4ccccc4)c3)c3ccc4c(c3)-c3cc5ccccc5cc3C4(C)C)cc21 ; CCC1(CC)c2ccccc2-c2ccc(N(c3ccc4c(c3)-c3cc5ccccc5cc3C4(C)C)c3ccccc3-c3ccccc3)cc21] 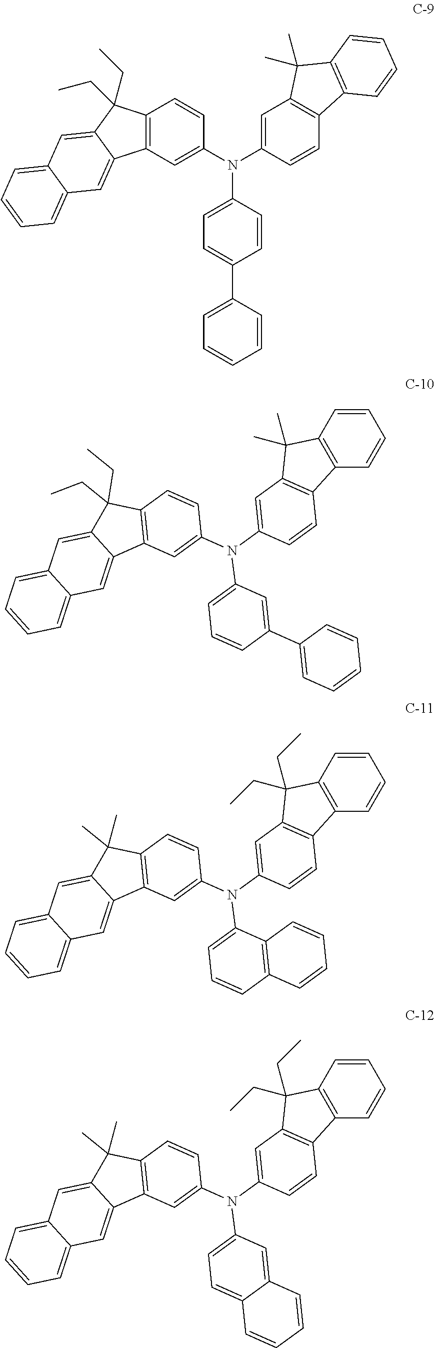 [SMILES: CCC1(CC)c2ccc(N(c3ccc(-c4ccccc4)cc3)c3ccc4c(c3)C(C)(C)c3ccccc3-4)cc2-c2cc3ccccc3cc21 ; CCC1(CC)c2ccc(N(c3cccc(-c4ccccc4)c3)c3ccc4c(c3)C(C)(C)c3ccccc3-4)cc2-c2cc3ccccc3cc21 ; CCC1(CC)c2ccccc2-c2ccc(N(c3ccc4c(c3)-c3cc5ccccc5cc3C4(C)C)c3cccc4ccccc34)cc21 ; CCC1(CC)c2ccccc2-c2ccc(N(c3ccc4c(c3)-c3cc5ccccc5cc3C4(C)C)c3ccc4ccccc4c3)cc21] 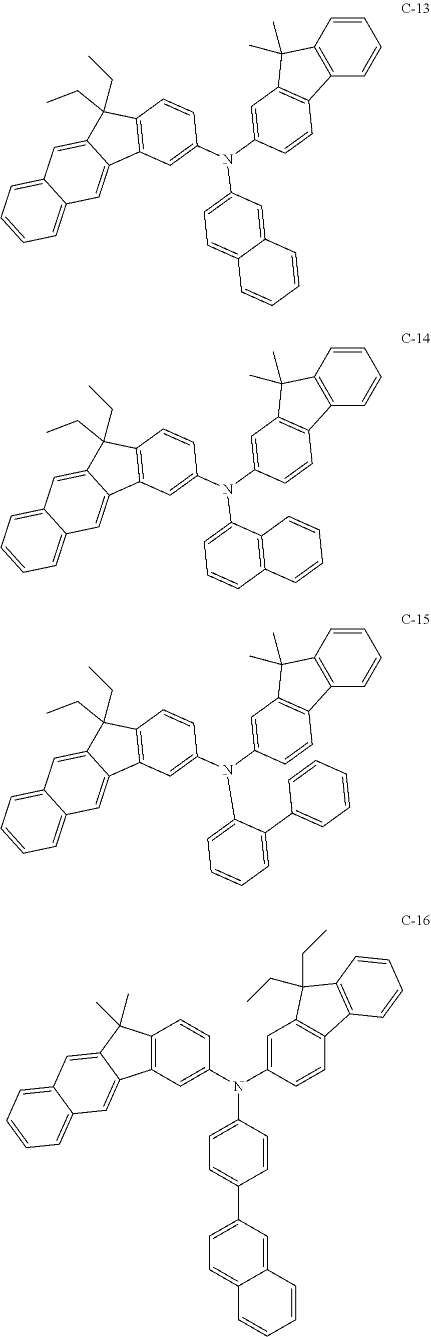 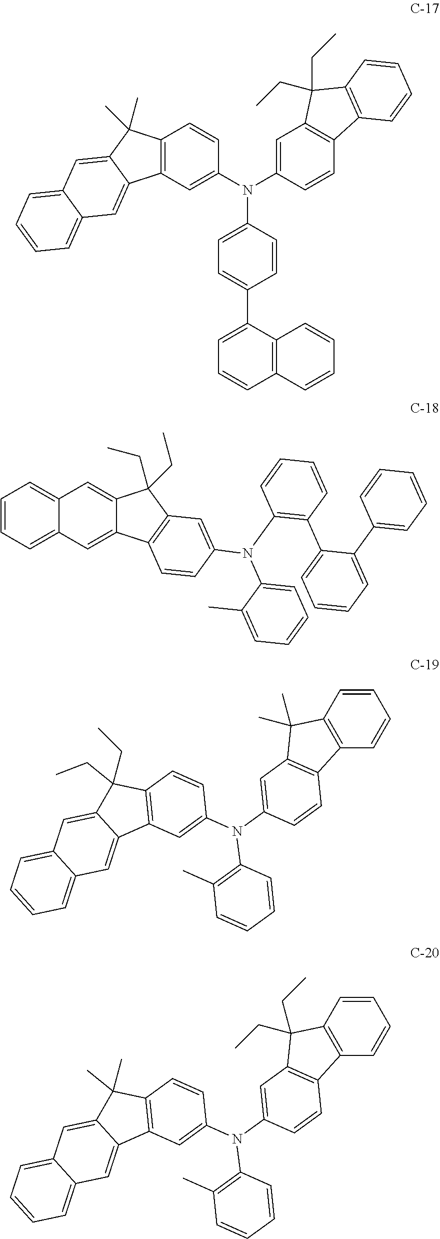 [SMILES: CCC1(CC)c2ccccc2-c2ccc(N(c3ccc(-c4cccc5ccccc45)cc3)c3ccc4c(c3)-c3cc5ccccc5cc3C4(C)C)cc21 ; CCC1(CC)c2cc(N(c3ccccc3C)c3ccccc3-c3ccccc3-c3ccccc3)ccc2-c2cc3ccccc3cc21 ; CCC1(CC)c2ccc(N(c3ccc4c(c3)C(C)(C)c3ccccc3-4)c3ccccc3C)cc2-c2cc3ccccc3cc21 ; CCC1(CC)c2ccccc2-c2ccc(N(c3ccc4c(c3)-c3cc5ccccc5cc3C4(C)C)c3ccccc3C)cc21] 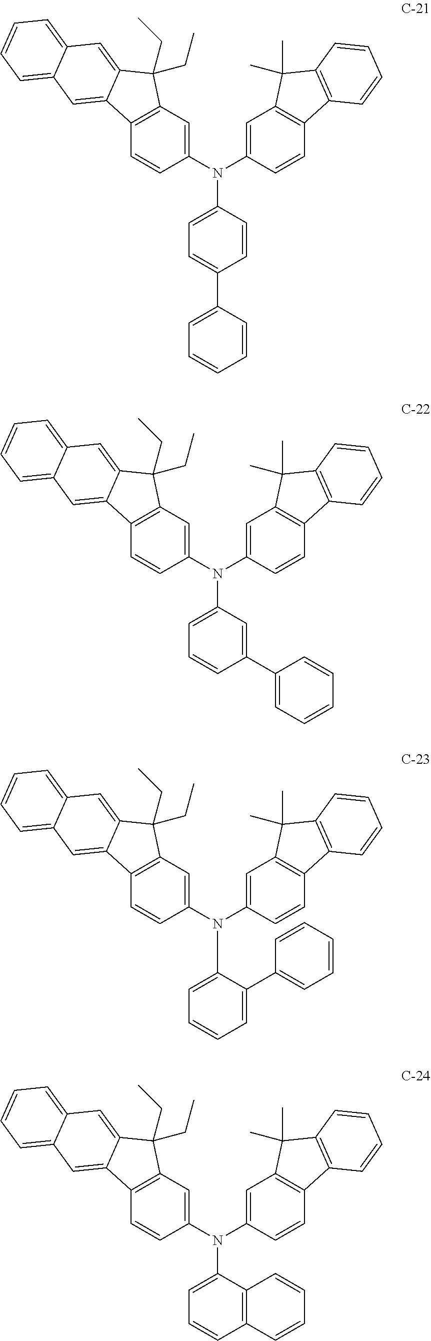 [SMILES: CCC1(CC)c2cc(N(c3ccc(-c4ccccc4)cc3)c3ccc4c(c3)C(C)(C)c3ccccc3-4)ccc2-c2cc3ccccc3cc21 ; CCC1(CC)c2cc(N(c3cccc(-c4ccccc4)c3)c3ccc4c(c3)C(C)(C)c3ccccc3-4)ccc2-c2cc3ccccc3cc21 ; CCC1(CC)c2cc(N(c3ccc4c(c3)C(C)(C)c3ccccc3-4)c3ccccc3-c3ccccc3)ccc2-c2cc3ccccc3cc21 ; CCC1(CC)c2cc(N(c3ccc4c(c3)C(C)(C)c3ccccc3-4)c3cccc4ccccc34)ccc2-c2cc3ccccc3cc21] 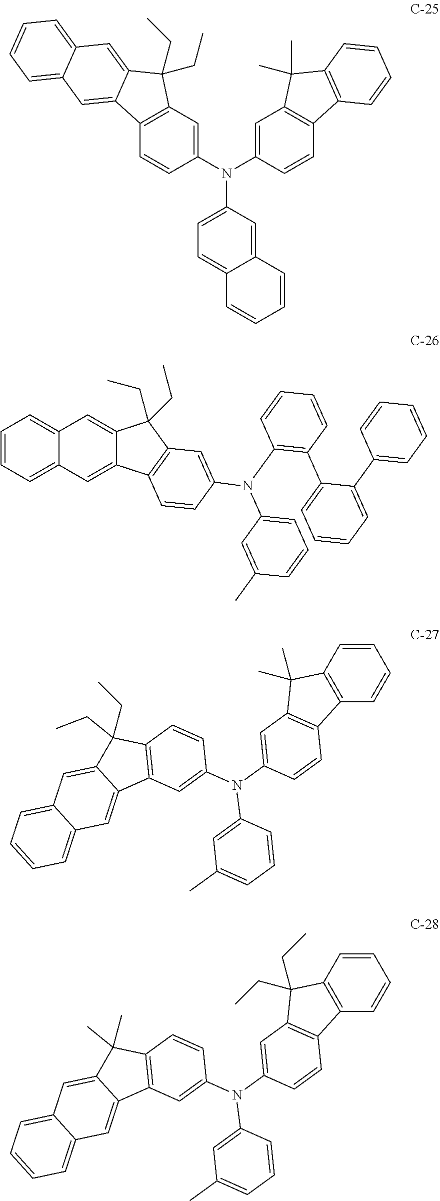 [SMILES: CCC1(CC)c2cc(N(c3ccc4c(c3)C(C)(C)c3ccccc3-4)c3ccc4ccccc4c3)ccc2-c2cc3ccccc3cc21 ; CCC1(CC)c2cc(N(c3cccc(C)c3)c3ccccc3-c3ccccc3-c3ccccc3)ccc2-c2cc3ccccc3cc21 ; CCC1(CC)c2ccc(N(c3cccc(C)c3)c3ccc4c(c3)C(C)(C)c3ccccc3-4)cc2-c2cc3ccccc3cc21 ; CCC1(CC)c2ccccc2-c2ccc(N(c3cccc(C)c3)c3ccc4c(c3)-c3cc5ccccc5cc3C4(C)C)cc21] 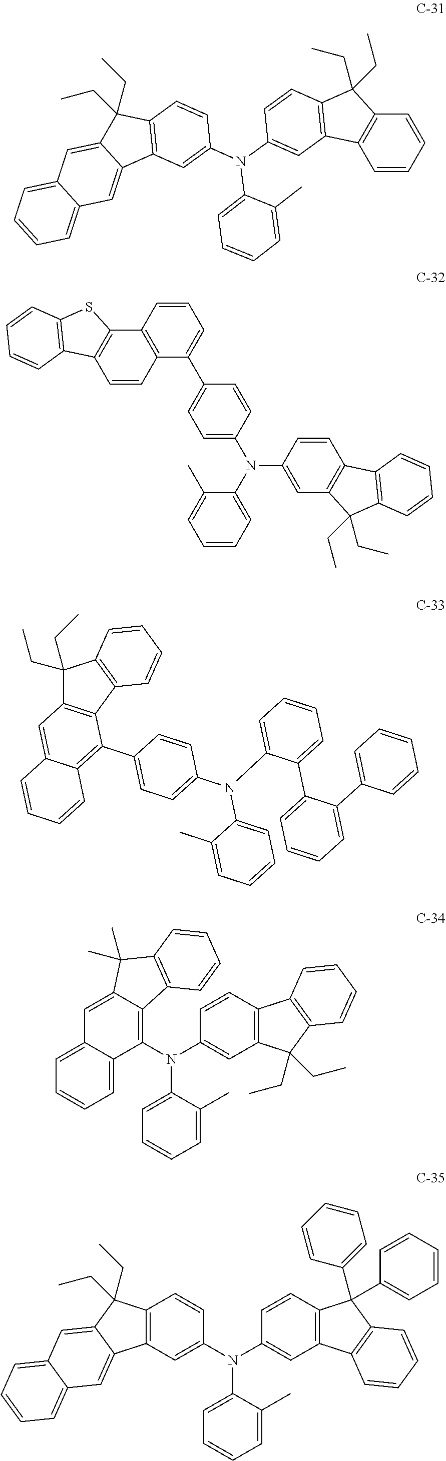 [SMILES: CCC1(CC)c2ccccc2-c2cc(N(c3ccc4c(c3)-c3cc5ccccc5cc3C4(CC)CC)c3ccccc3C)ccc21 ; CCC1(CC)c2ccccc2-c2ccc(N(c3ccc(-c4cccc5c4ccc4c6ccccc6sc54)cc3)c3ccccc3C)cc21 ; CCC1(CC)c2ccccc2-c2c1cc1ccccc1c2-c1ccc(N(c2ccccc2C)c2ccccc2-c2ccccc2-c2ccccc2)cc1 ; CCC1(CC)c2ccccc2-c2ccc(N(c3ccccc3C)c3c4c(cc5ccccc35)C(C)(C)c3ccccc3-4)cc21 ; CCC1(CC)c2ccc(N(c3ccc4c(c3)-c3ccccc3C4(c3ccccc3)c3ccccc3)c3ccccc3C)cc2-c2cc3ccccc3cc21] 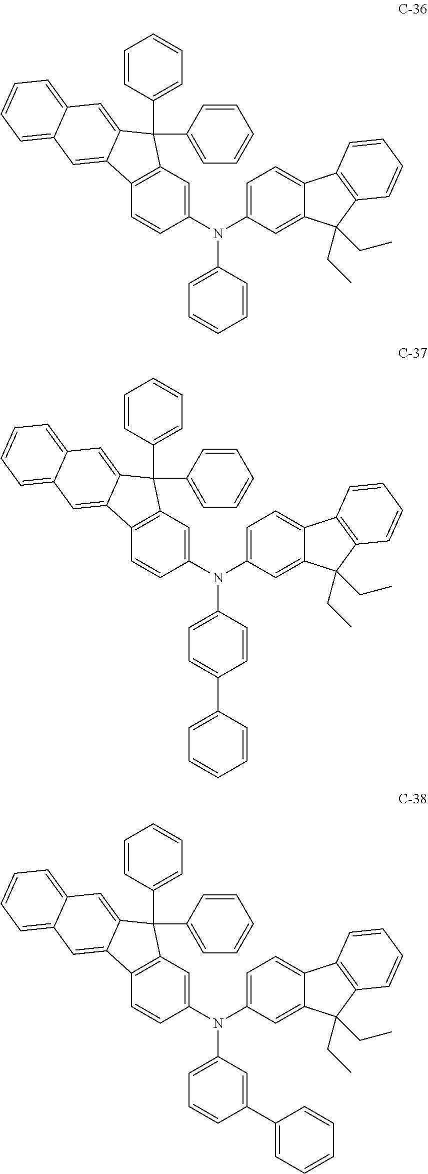 [SMILES: CCC1(CC)c2ccccc2-c2ccc(N(c3ccccc3)c3ccc4c(c3)C(c3ccccc3)(c3ccccc3)c3cc5ccccc5cc3-4)cc21 ; CCC1(CC)c2ccccc2-c2ccc(N(c3ccc(-c4ccccc4)cc3)c3ccc4c(c3)C(c3ccccc3)(c3ccccc3)c3cc5ccccc5cc3-4)cc21 ; CCC1(CC)c2ccccc2-c2ccc(N(c3cccc(-c4ccccc4)c3)c3ccc4c(c3)C(c3ccccc3)(c3ccccc3)c3cc5ccccc5cc3-4)cc21] 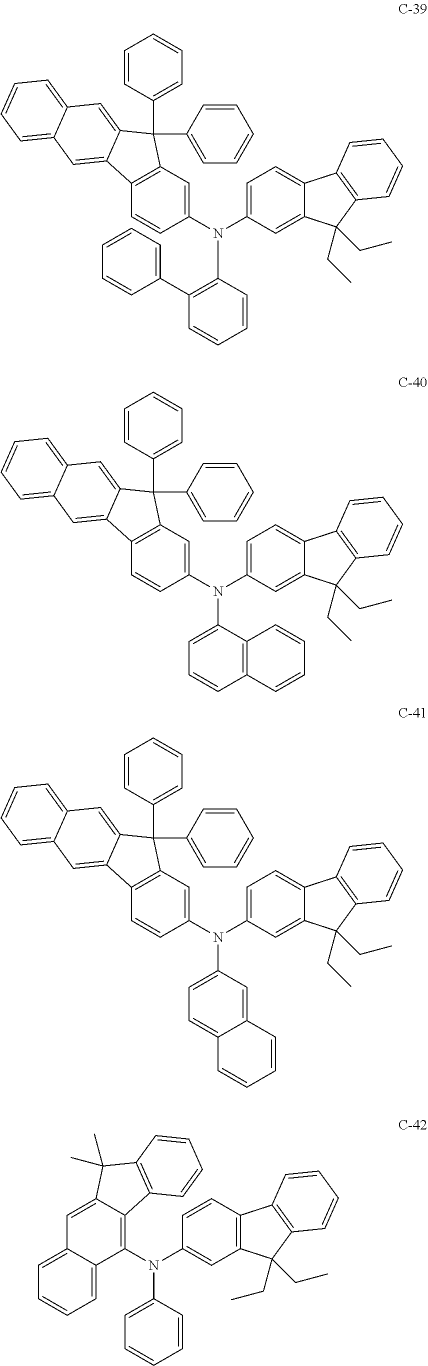 [SMILES: CCC1(CC)c2ccccc2-c2ccc(N(c3ccc4c(c3)C(c3ccccc3)(c3ccccc3)c3cc5ccccc5cc3-4)c3ccccc3-c3ccccc3)cc21 ; CCC1(CC)c2ccccc2-c2ccc(N(c3ccc4c(c3)C(c3ccccc3)(c3ccccc3)c3cc5ccccc5cc3-4)c3cccc4ccccc34)cc21 ; CCC1(CC)c2ccccc2-c2ccc(N(c3ccc4c(c3)C(c3ccccc3)(c3ccccc3)c3cc5ccccc5cc3-4)c3ccc4ccccc4c3)cc21 ; CCC1(CC)c2ccccc2-c2ccc(N(c3ccccc3)c3c4c(cc5ccccc35)C(C)(C)c3ccccc3-4)cc21] 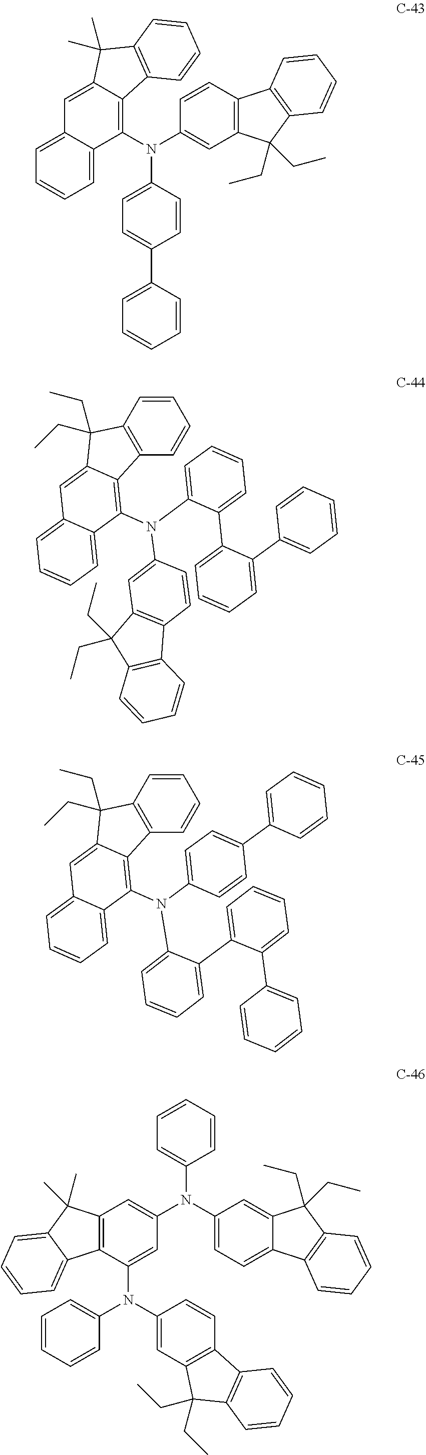 [SMILES: CCC1(CC)c2ccccc2-c2ccc(N(c3ccc(-c4ccccc4)cc3)c3c4c(cc5ccccc35)C(C)(C)c3ccccc3-4)cc21 ; CCC1(CC)c2ccccc2-c2ccc(N(c3ccccc3-c3ccccc3-c3ccccc3)c3c4c(cc5ccccc35)C(CC)(CC)c3ccccc3-4)cc21 ; CCC1(CC)c2ccccc2-c2c1cc1ccccc1c2N(c1ccc(-c2ccccc2)cc1)c1ccccc1-c1ccccc1-c1ccccc1 ; CCC1(CC)c2ccccc2-c2ccc(N(c3ccccc3)c3cc(N(c4ccccc4)c4ccc5c(c4)C(CC)(CC)c4ccccc4-5)c4c(c3)C(C)(C)c3ccccc3-4)cc21] 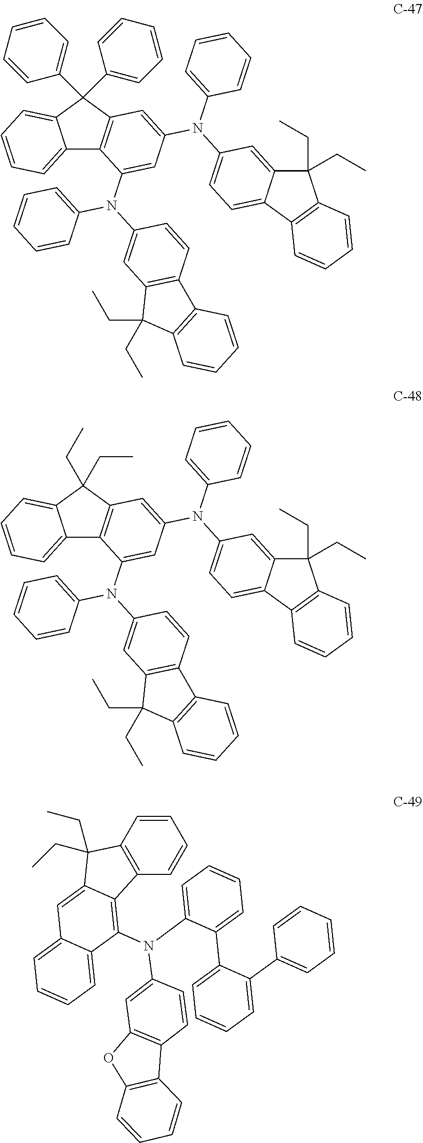 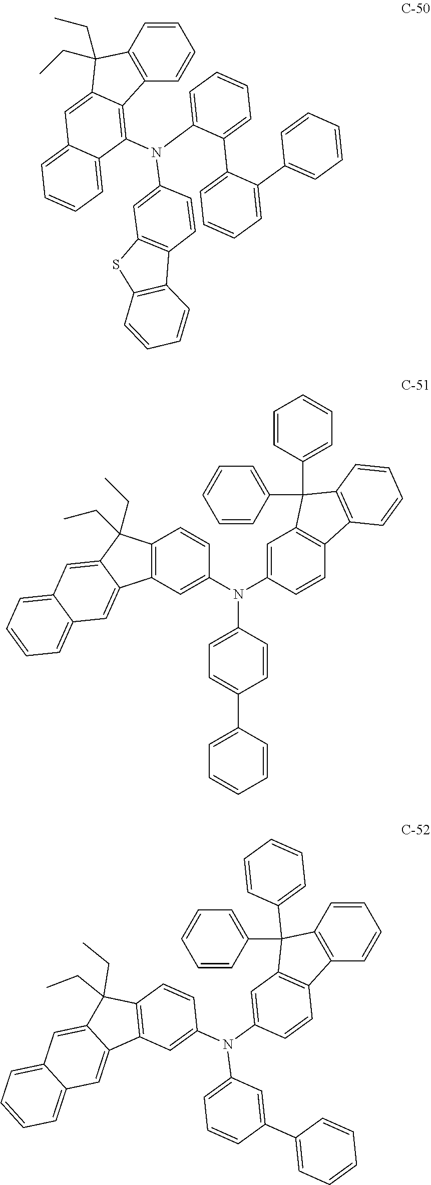 [SMILES: CCC1(CC)c2ccccc2-c2c1cc1ccccc1c2N(c1ccc2c(c1)sc1ccccc12)c1ccccc1-c1ccccc1-c1ccccc1 ; CCC1(CC)c2ccc(N(c3ccc(-c4ccccc4)cc3)c3ccc4c(c3)C(c3ccccc3)(c3ccccc3)c3ccccc3-4)cc2-c2cc3ccccc3cc21 ; CCC1(CC)c2ccc(N(c3cccc(-c4ccccc4)c3)c3ccc4c(c3)C(c3ccccc3)(c3ccccc3)c3ccccc3-4)cc2-c2cc3ccccc3cc21] 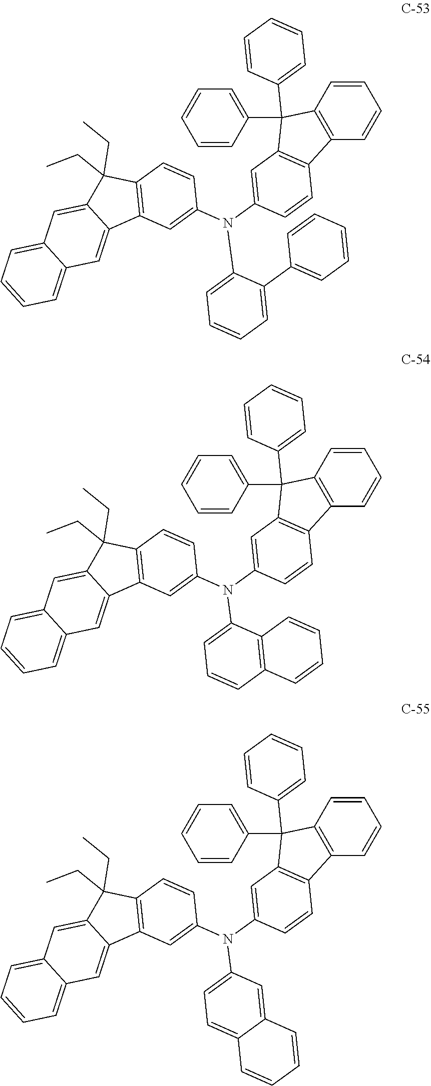 [SMILES: CCC1(CC)c2ccc(N(c3ccc4c(c3)C(c3ccccc3)(c3ccccc3)c3ccccc3-4)c3ccccc3-c3ccccc3)cc2-c2cc3ccccc3cc21 ; CCC1(CC)c2ccc(N(c3ccc4c(c3)C(c3ccccc3)(c3ccccc3)c3ccccc3-4)c3cccc4ccccc34)cc2-c2cc3ccccc3cc21 ; CCC1(CC)c2ccc(N(c3ccc4c(c3)C(c3ccccc3)(c3ccccc3)c3ccccc3-4)c3ccc4ccccc4c3)cc2-c2cc3ccccc3cc21] 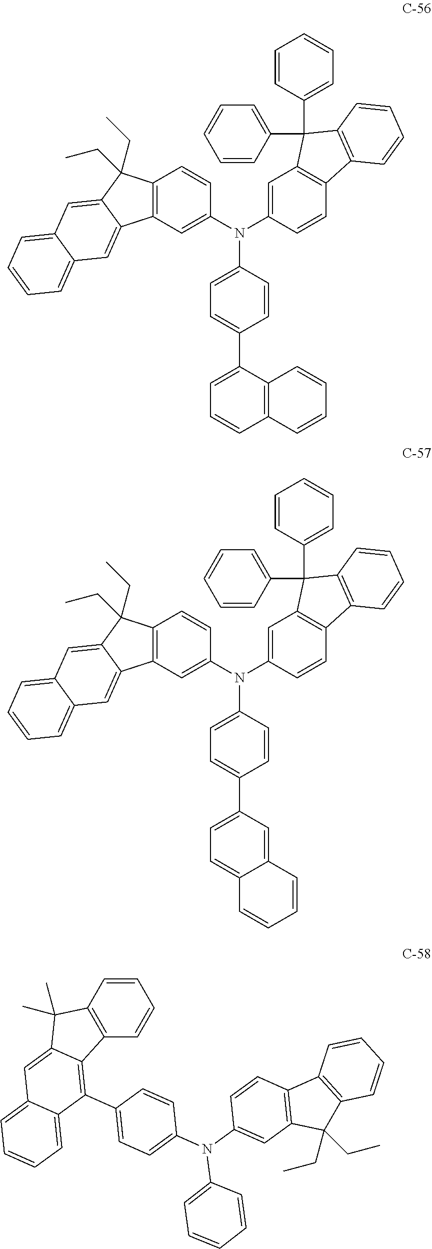 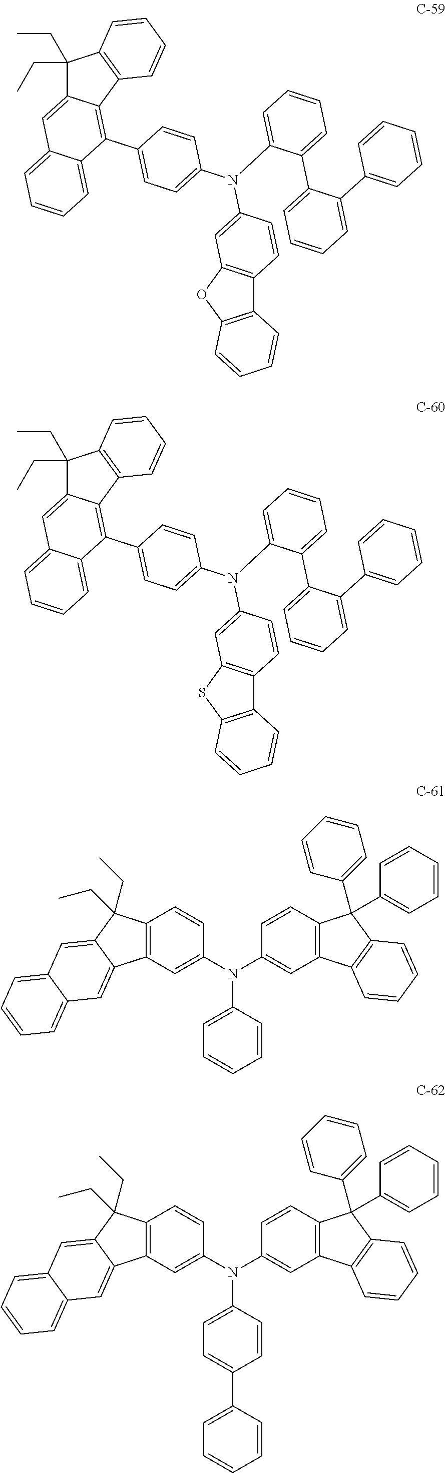 [SMILES: CCC1(CC)c2ccccc2-c2c1cc1ccccc1c2-c1ccc(N(c2ccc3c(c2)oc2ccccc23)c2ccccc2-c2ccccc2-c2ccccc2)cc1 ; CCC1(CC)c2ccccc2-c2c1cc1ccccc1c2-c1ccc(N(c2ccc3c(c2)sc2ccccc23)c2ccccc2-c2ccccc2-c2ccccc2)cc1 ; CCC1(CC)c2ccc(N(c3ccccc3)c3ccc4c(c3)-c3ccccc3C4(c3ccccc3)c3ccccc3)cc2-c2cc3ccccc3cc21 ; CCC1(CC)c2ccc(N(c3ccc(-c4ccccc4)cc3)c3ccc4c(c3)-c3ccccc3C4(c3ccccc3)c3ccccc3)cc2-c2cc3ccccc3cc21] 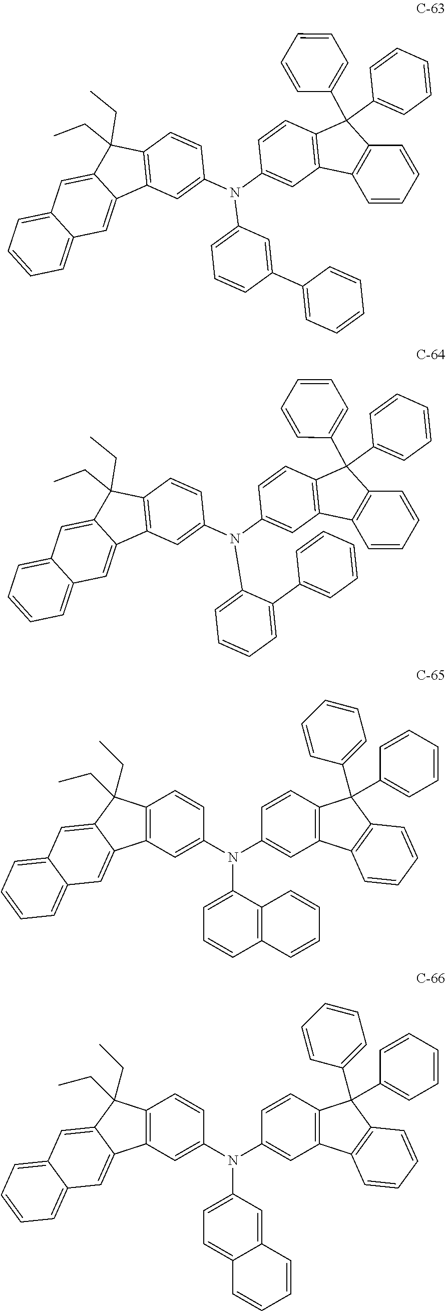 [SMILES: CCC1(CC)c2ccc(N(c3cccc(-c4ccccc4)c3)c3ccc4c(c3)-c3ccccc3C4(c3ccccc3)c3ccccc3)cc2-c2cc3ccccc3cc21 ; CCC1(CC)c2ccc(N(c3ccc4c(c3)-c3ccccc3C4(c3ccccc3)c3ccccc3)c3ccccc3-c3ccccc3)cc2-c2cc3ccccc3cc21 ; CCC1(CC)c2ccc(N(c3ccc4c(c3)-c3ccccc3C4(c3ccccc3)c3ccccc3)c3cccc4ccccc34)cc2-c2cc3ccccc3cc21 ; CCC1(CC)c2ccc(N(c3ccc4c(c3)-c3ccccc3C4(c3ccccc3)c3ccccc3)c3ccc4ccccc4c3)cc2-c2cc3ccccc3cc21] 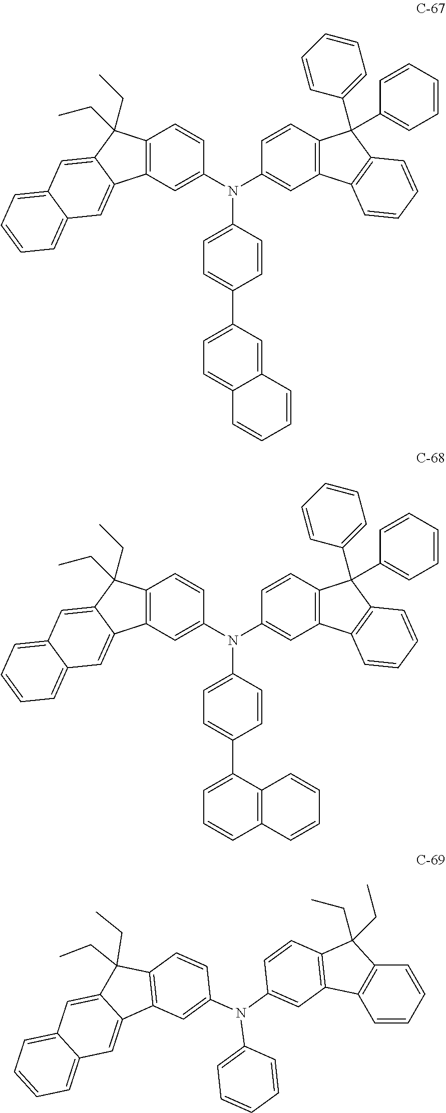 [SMILES: CCC1(CC)c2ccc(N(c3ccc(-c4ccc5ccccc5c4)cc3)c3ccc4c(c3)-c3ccccc3C4(c3ccccc3)c3ccccc3)cc2-c2cc3ccccc3cc21 ; CCC1(CC)c2ccc(N(c3ccc(-c4cccc5ccccc45)cc3)c3ccc4c(c3)-c3ccccc3C4(c3ccccc3)c3ccccc3)cc2-c2cc3ccccc3cc21 ; CCC1(CC)c2ccccc2-c2cc(N(c3ccccc3)c3ccc4c(c3)-c3cc5ccccc5cc3C4(CC)CC)ccc21] 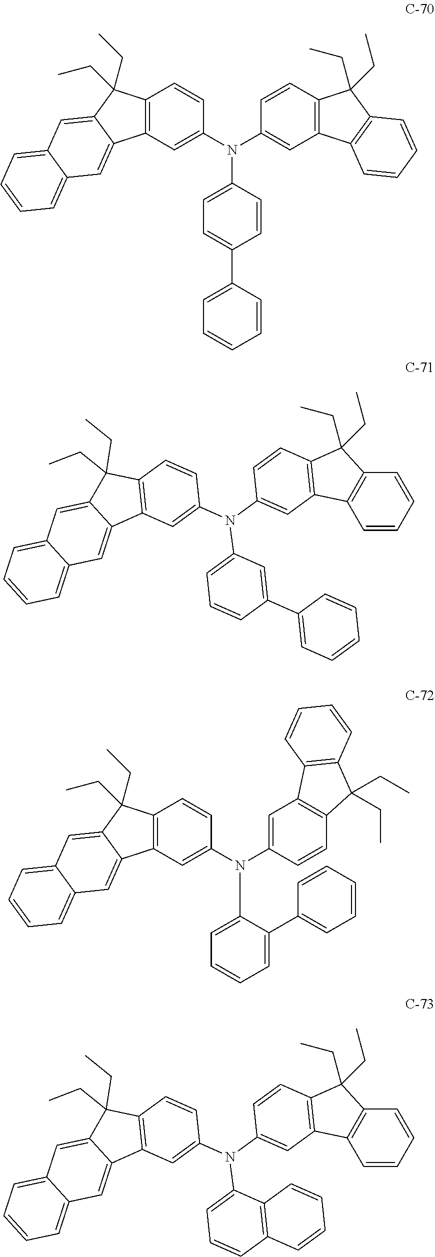 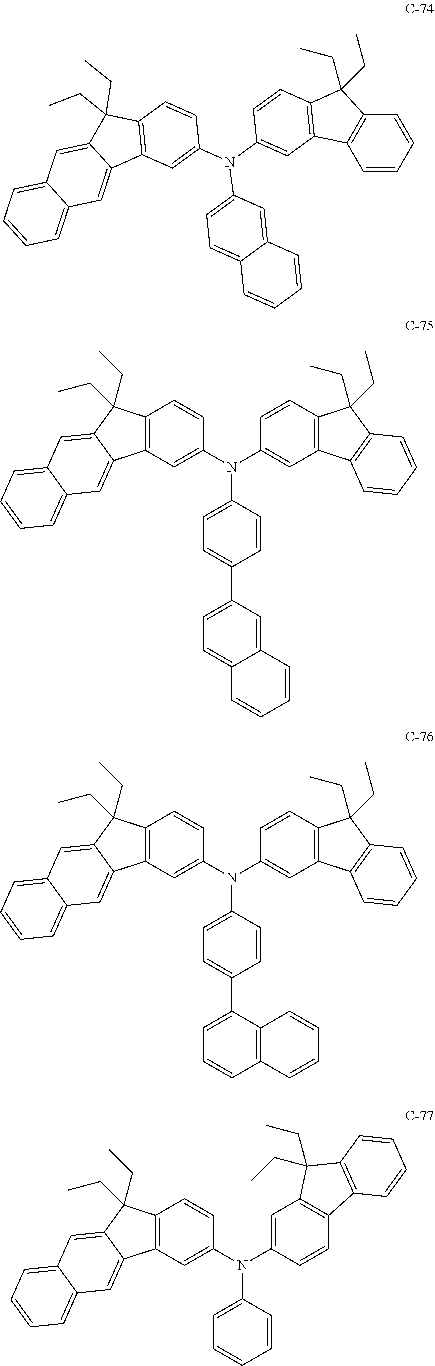 [SMILES: CCC1(CC)c2ccccc2-c2cc(N(c3ccc4c(c3)-c3cc5ccccc5cc3C4(CC)CC)c3ccc4ccccc4c3)ccc21 ; CCC1(CC)c2ccccc2-c2cc(N(c3ccc(-c4ccc5ccccc5c4)cc3)c3ccc4c(c3)-c3cc5ccccc5cc3C4(CC)CC)ccc21 ; CCC1(CC)c2ccccc2-c2cc(N(c3ccc(-c4cccc5ccccc45)cc3)c3ccc4c(c3)-c3cc5ccccc5cc3C4(CC)CC)ccc21 ; CCC1(CC)c2ccccc2-c2ccc(N(c3ccccc3)c3ccc4c(c3)-c3cc5ccccc5cc3C4(CC)CC)cc21] 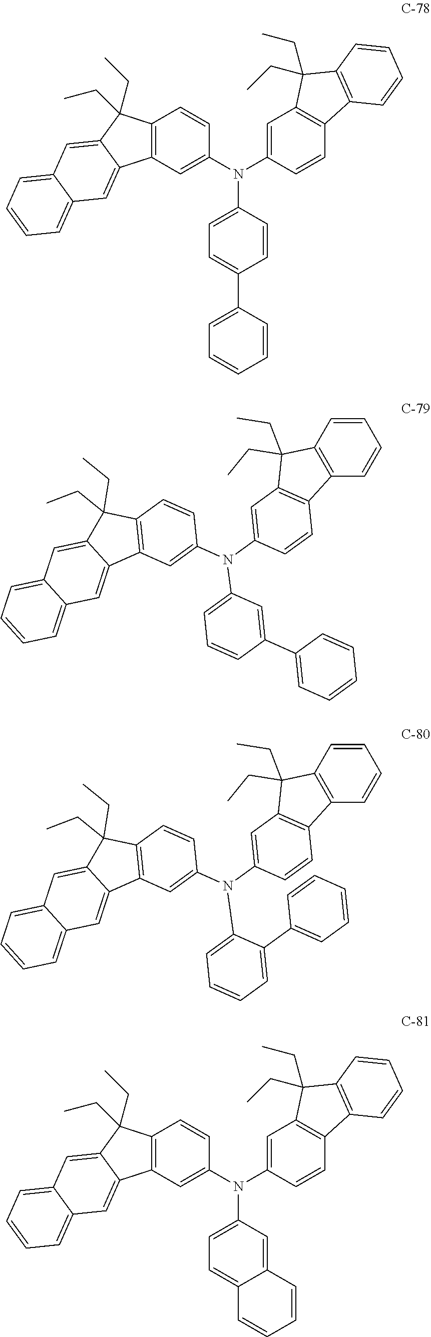 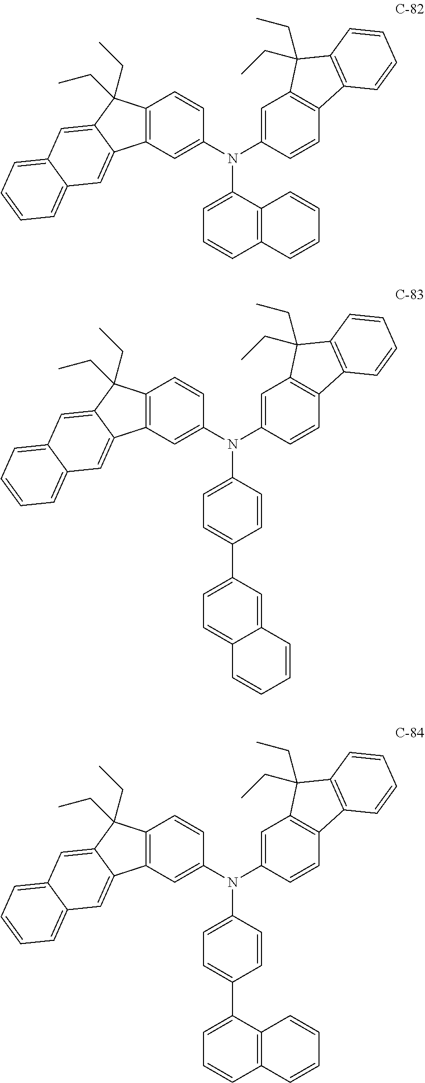 [SMILES: CCC1(CC)c2ccccc2-c2ccc(N(c3ccc4c(c3)-c3cc5ccccc5cc3C4(CC)CC)c3cccc4ccccc34)cc21 ; CCC1(CC)c2ccccc2-c2ccc(N(c3ccc(-c4ccc5ccccc5c4)cc3)c3ccc4c(c3)-c3cc5ccccc5cc3C4(CC)CC)cc21 ; CCC1(CC)c2ccccc2-c2ccc(N(c3ccc(-c4cccc5ccccc45)cc3)c3ccc4c(c3)-c3cc5ccccc5cc3C4(CC)CC)cc21] 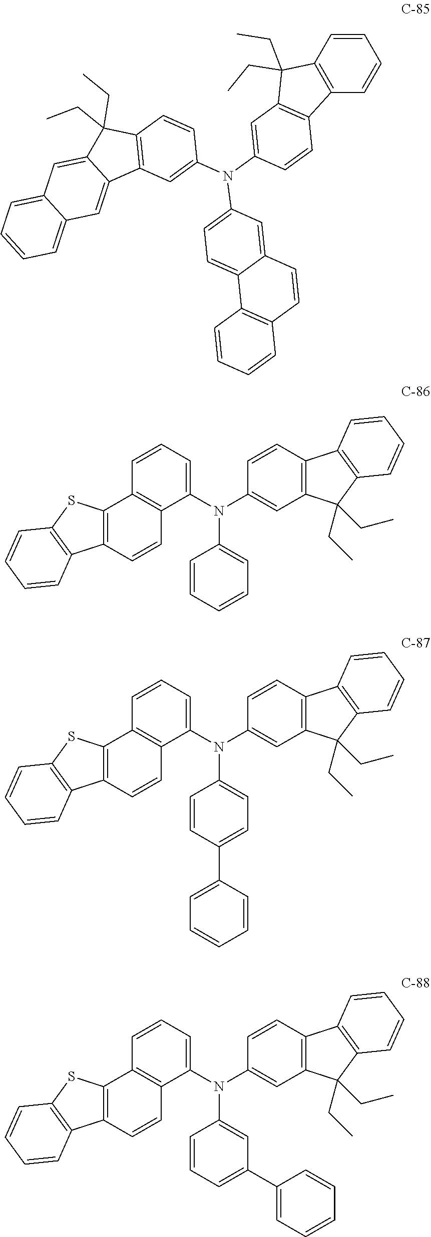 [SMILES: CCC1(CC)c2ccccc2-c2ccc(N(c3ccc4c(c3)-c3cc5ccccc5cc3C4(CC)CC)c3ccc4c(ccc5ccccc54)c3)cc21 ; CCC1(CC)c2ccccc2-c2ccc(N(c3ccccc3)c3cccc4c3ccc3c5ccccc5sc43)cc21 ; CCC1(CC)c2ccccc2-c2ccc(N(c3ccc(-c4ccccc4)cc3)c3cccc4c3ccc3c5ccccc5sc43)cc21 ; CCC1(CC)c2ccccc2-c2ccc(N(c3cccc(-c4ccccc4)c3)c3cccc4c3ccc3c5ccccc5sc43)cc21] 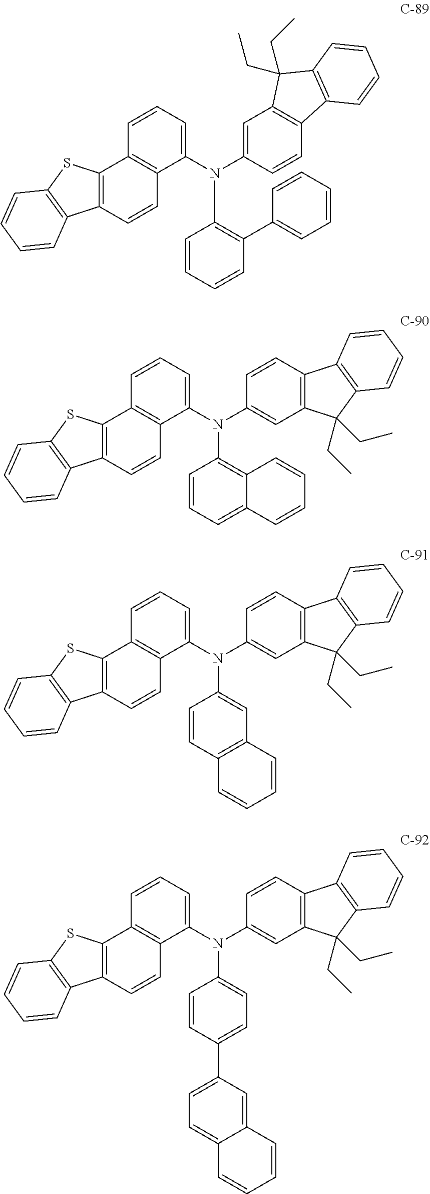 [SMILES: CCC1(CC)c2ccccc2-c2ccc(N(c3ccccc3-c3ccccc3)c3cccc4c3ccc3c5ccccc5sc43)cc21 ; CCC1(CC)c2ccccc2-c2ccc(N(c3cccc4ccccc34)c3cccc4c3ccc3c5ccccc5sc43)cc21 ; CCC1(CC)c2ccccc2-c2ccc(N(c3ccc4ccccc4c3)c3cccc4c3ccc3c5ccccc5sc43)cc21 ; CCC1(CC)c2ccccc2-c2ccc(N(c3ccc(-c4ccc5ccccc5c4)cc3)c3cccc4c3ccc3c5ccccc5sc43)cc21] 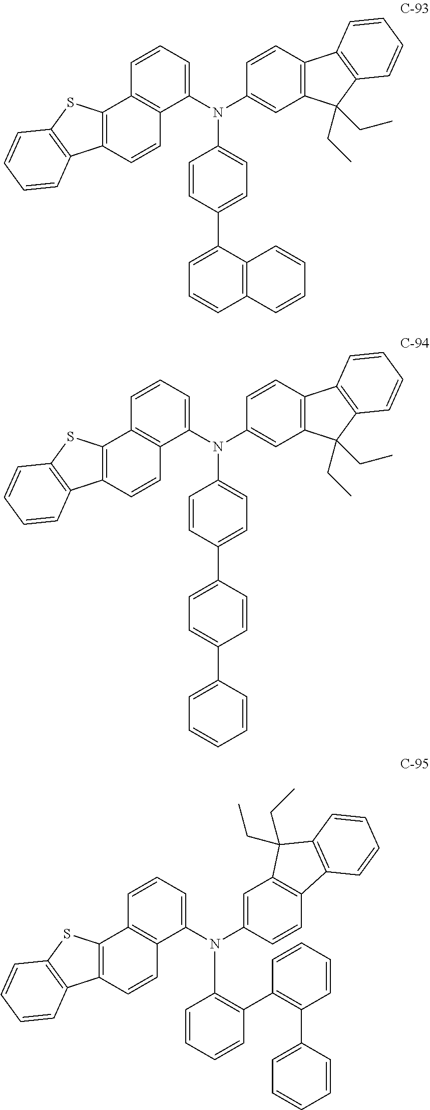 [SMILES: CCC1(CC)c2ccccc2-c2ccc(N(c3ccc(-c4cccc5ccccc45)cc3)c3cccc4c3ccc3c5ccccc5sc43)cc21 ; CCC1(CC)c2ccccc2-c2ccc(N(c3ccc(-c4ccc(-c5ccccc5)cc4)cc3)c3cccc4c3ccc3c5ccccc5sc43)cc21 ; CCC1(CC)c2ccccc2-c2ccc(N(c3ccccc3-c3ccccc3-c3ccccc3)c3cccc4c3ccc3c5ccccc5sc43)cc21] 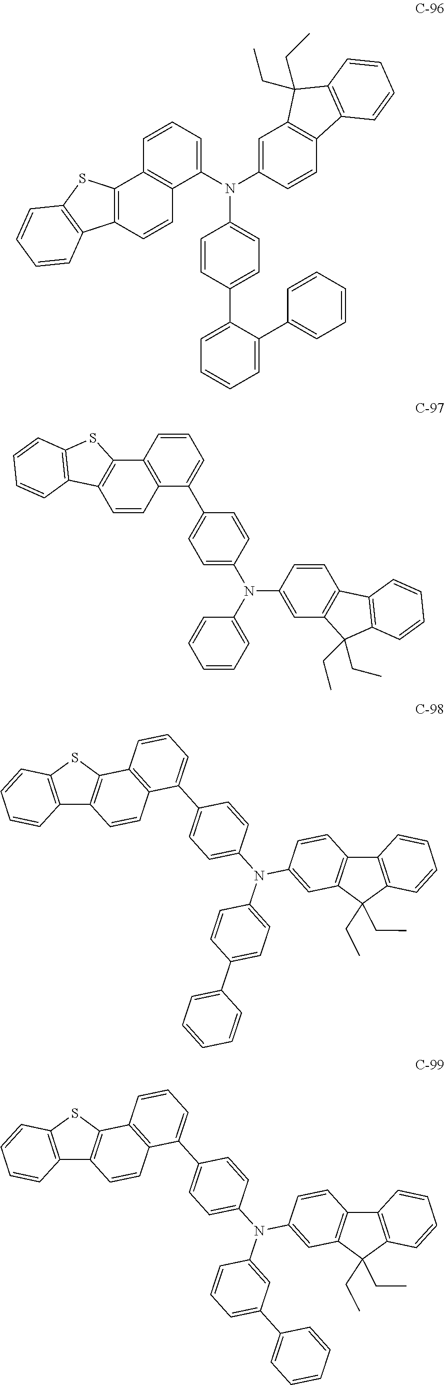 [SMILES: CCC1(CC)c2ccccc2-c2ccc(N(c3ccc(-c4ccccc4-c4ccccc4)cc3)c3cccc4c3ccc3c5ccccc5sc43)cc21 ; CCC1(CC)c2ccccc2-c2ccc(N(c3ccccc3)c3ccc(-c4cccc5c4ccc4c6ccccc6sc54)cc3)cc21 ; CCC1(CC)c2ccccc2-c2ccc(N(c3ccc(-c4ccccc4)cc3)c3ccc(-c4cccc5c4ccc4c6ccccc6sc54)cc3)cc21 ; CCC1(CC)c2ccccc2-c2ccc(N(c3ccc(-c4cccc5c4ccc4c6ccccc6sc54)cc3)c3cccc(-c4ccccc4)c3)cc21] 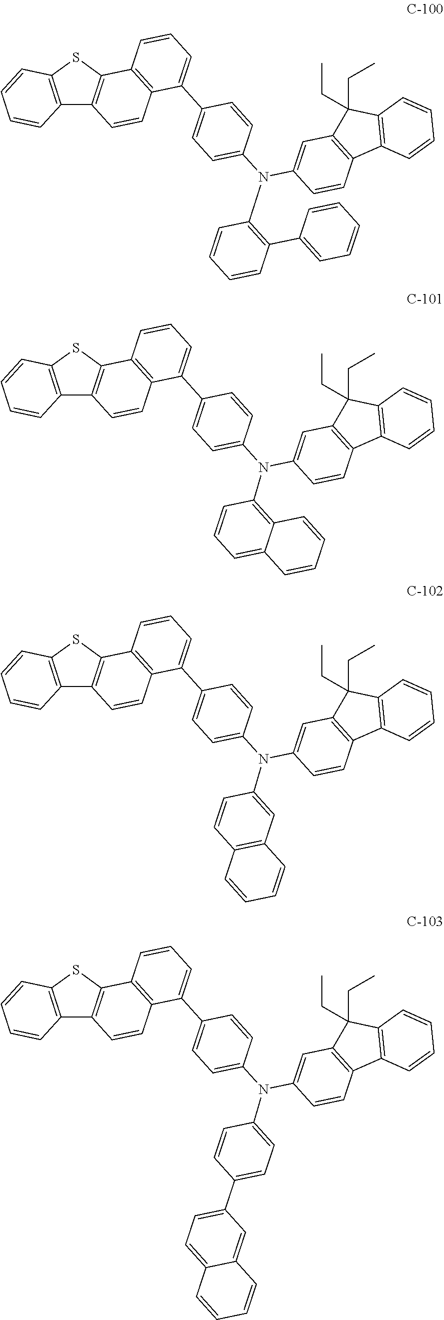 [SMILES: CCC1(CC)c2ccccc2-c2ccc(N(c3ccc(-c4cccc5c4ccc4c6ccccc6sc54)cc3)c3ccccc3-c3ccccc3)cc21 ; CCC1(CC)c2ccccc2-c2ccc(N(c3ccc(-c4cccc5c4ccc4c6ccccc6sc54)cc3)c3cccc4ccccc34)cc21 ; CCC1(CC)c2ccccc2-c2ccc(N(c3ccc(-c4cccc5c4ccc4c6ccccc6sc54)cc3)c3ccc4ccccc4c3)cc21 ; CCC1(CC)c2ccccc2-c2ccc(N(c3ccc(-c4ccc5ccccc5c4)cc3)c3ccc(-c4cccc5c4ccc4c6ccccc6sc54)cc3)cc21] 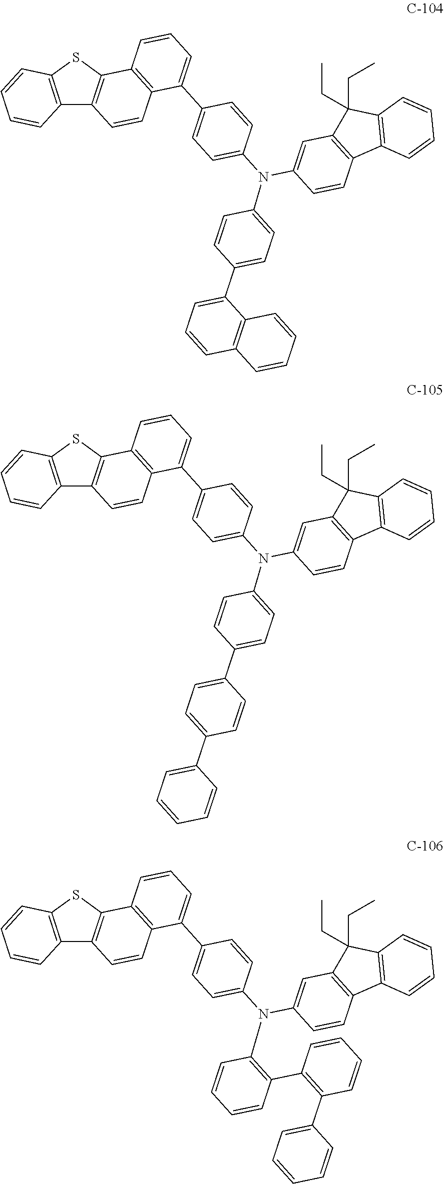 [SMILES: CCC1(CC)c2ccccc2-c2ccc(N(c3ccc(-c4cccc5ccccc45)cc3)c3ccc(-c4cccc5c4ccc4c6ccccc6sc54)cc3)cc21 ; CCC1(CC)c2ccccc2-c2ccc(N(c3ccc(-c4ccc(-c5ccccc5)cc4)cc3)c3ccc(-c4cccc5c4ccc4c6ccccc6sc54)cc3)cc21 ; CCC1(CC)c2ccccc2-c2ccc(N(c3ccc(-c4cccc5c4ccc4c6ccccc6sc54)cc3)c3ccccc3-c3ccccc3-c3ccccc3)cc21] 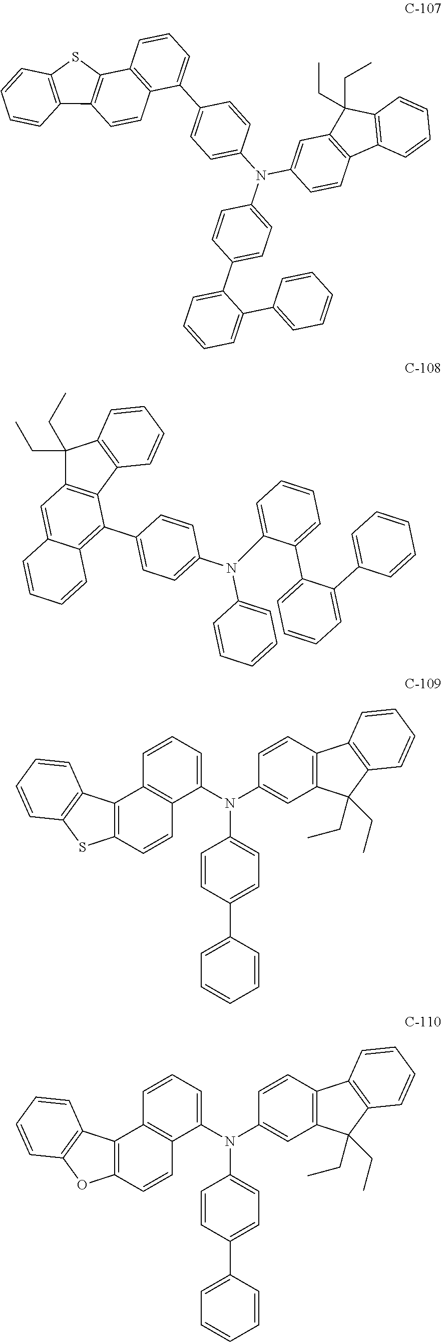 [SMILES: CCC1(CC)c2ccccc2-c2ccc(N(c3ccc(-c4ccccc4-c4ccccc4)cc3)c3ccc(-c4cccc5c4ccc4c6ccccc6sc54)cc3)cc21 ; CCC1(CC)c2ccccc2-c2c1cc1ccccc1c2-c1ccc(N(c2ccccc2)c2ccccc2-c2ccccc2-c2ccccc2)cc1 ; CCC1(CC)c2ccccc2-c2ccc(N(c3ccc(-c4ccccc4)cc3)c3cccc4c3ccc3sc5ccccc5c34)cc21 ; CCC1(CC)c2ccccc2-c2ccc(N(c3ccc(-c4ccccc4)cc3)c3cccc4c3ccc3oc5ccccc5c34)cc21] 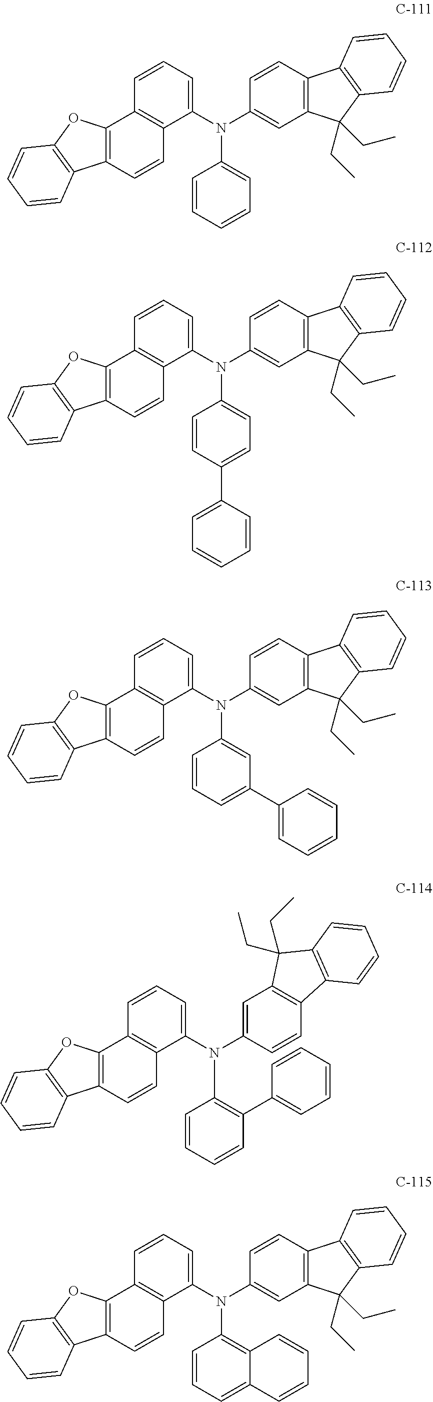 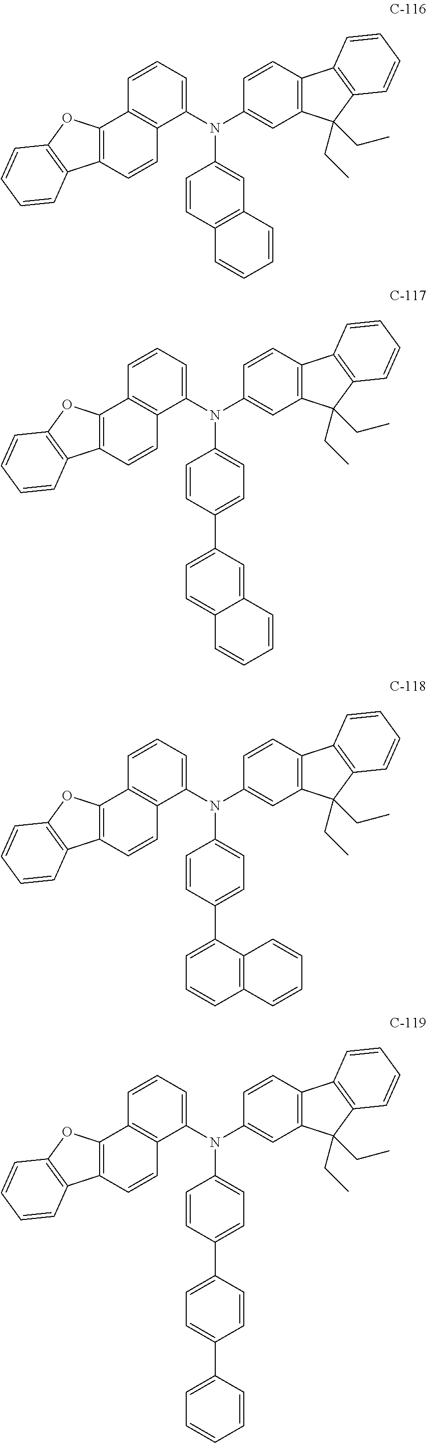 [SMILES: CCC1(CC)c2ccccc2-c2ccc(N(c3ccc4ccccc4c3)c3cccc4c3ccc3c5ccccc5oc43)cc21 ; CCC1(CC)c2ccccc2-c2ccc(N(c3ccc(-c4ccc5ccccc5c4)cc3)c3cccc4c3ccc3c5ccccc5oc43)cc21 ; CCC1(CC)c2ccccc2-c2ccc(N(c3ccc(-c4cccc5ccccc45)cc3)c3cccc4c3ccc3c5ccccc5oc43)cc21 ; CCC1(CC)c2ccccc2-c2ccc(N(c3ccc(-c4ccc(-c5ccccc5)cc4)cc3)c3cccc4c3ccc3c5ccccc5oc43)cc21] 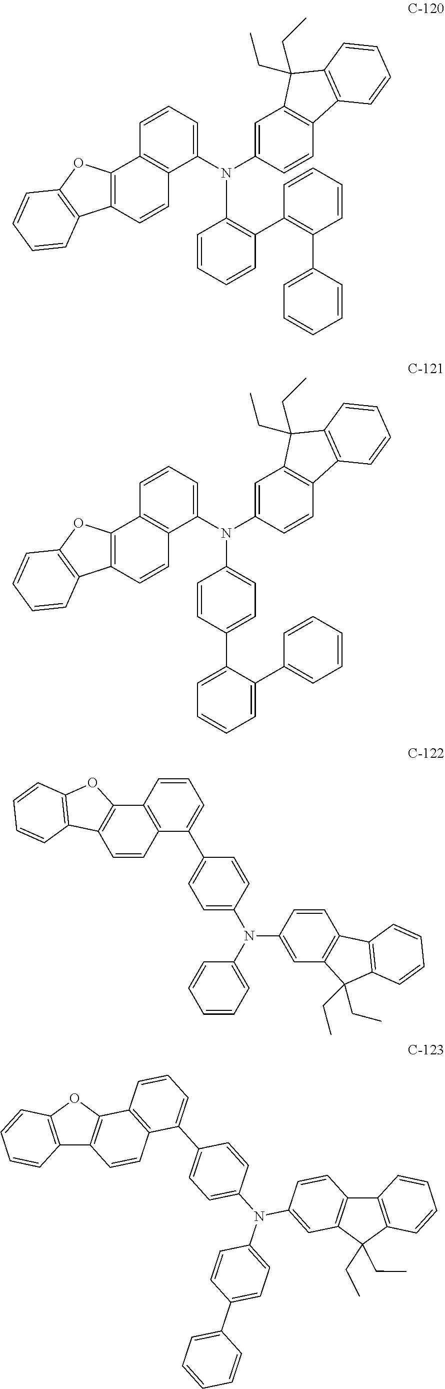 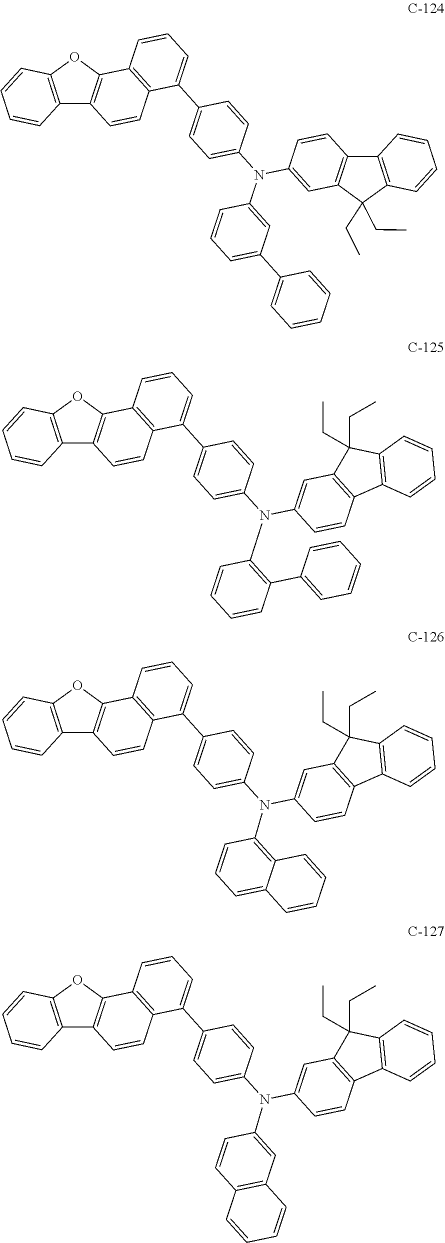 [SMILES: CCC1(CC)c2ccccc2-c2ccc(N(c3ccc(-c4cccc5c4ccc4c6ccccc6oc54)cc3)c3cccc(-c4ccccc4)c3)cc21 ; CCC1(CC)c2ccccc2-c2ccc(N(c3ccc(-c4cccc5c4ccc4c6ccccc6oc54)cc3)c3ccccc3-c3ccccc3)cc21 ; CCC1(CC)c2ccccc2-c2ccc(N(c3ccc(-c4cccc5c4ccc4c6ccccc6oc54)cc3)c3cccc4ccccc34)cc21 ; CCC1(CC)c2ccccc2-c2ccc(N(c3ccc(-c4cccc5c4ccc4c6ccccc6oc54)cc3)c3ccc4ccccc4c3)cc21] 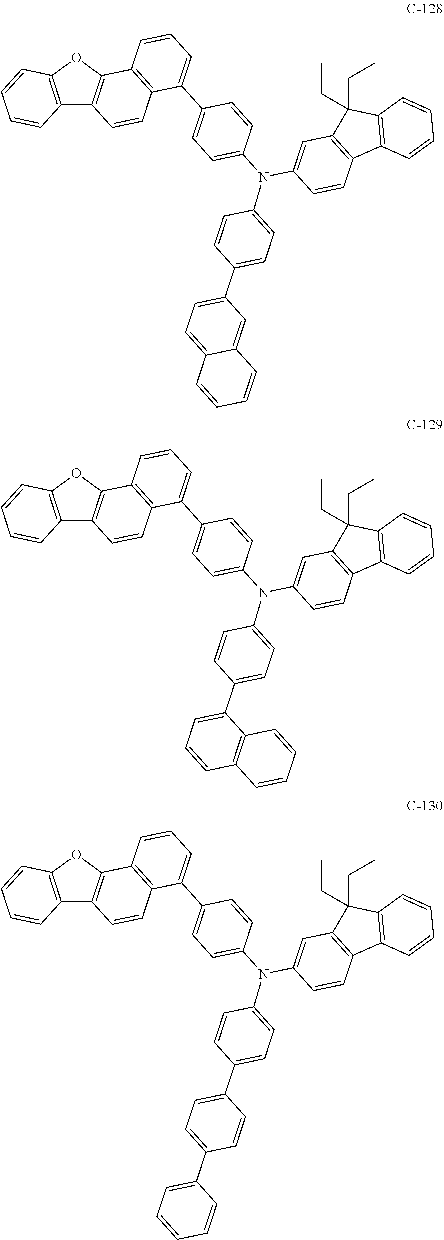 [SMILES: CCC1(CC)c2ccccc2-c2ccc(N(c3ccc(-c4ccc5ccccc5c4)cc3)c3ccc(-c4cccc5c4ccc4c6ccccc6oc54)cc3)cc21 ; CCC1(CC)c2ccccc2-c2ccc(N(c3ccc(-c4cccc5ccccc45)cc3)c3ccc(-c4cccc5c4ccc4c6ccccc6oc54)cc3)cc21 ; CCC1(CC)c2ccccc2-c2ccc(N(c3ccc(-c4ccc(-c5ccccc5)cc4)cc3)c3ccc(-c4cccc5c4ccc4c6ccccc6oc54)cc3)cc21] 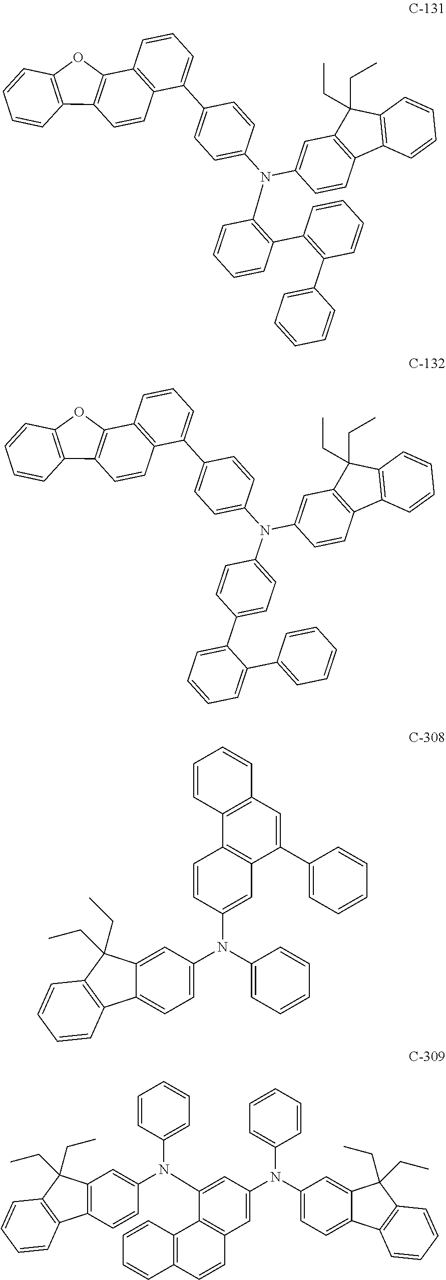 [SMILES: CCC1(CC)c2ccccc2-c2ccc(N(c3ccc(-c4cccc5c4ccc4c6ccccc6oc54)cc3)c3ccccc3-c3ccccc3-c3ccccc3)cc21 ; CCC1(CC)c2ccccc2-c2ccc(N(c3ccc(-c4ccccc4-c4ccccc4)cc3)c3ccc(-c4cccc5c4ccc4c6ccccc6oc54)cc3)cc21 ; CCC1(CC)c2ccccc2-c2ccc(N(c3ccccc3)c3ccc4c(c3)c(-c3ccccc3)cc3ccccc34)cc21 ; CCC1(CC)c2ccccc2-c2ccc(N(c3ccccc3)c3cc(N(c4ccccc4)c4ccc5c(c4)C(CC)(CC)c4ccccc4-5)c4c(ccc5ccccc54)c3)cc21] 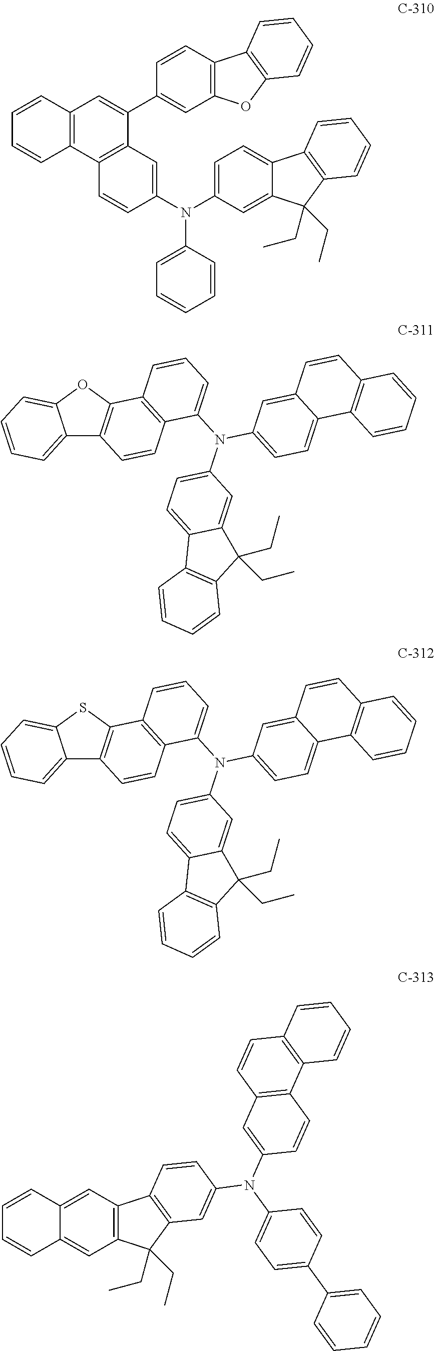 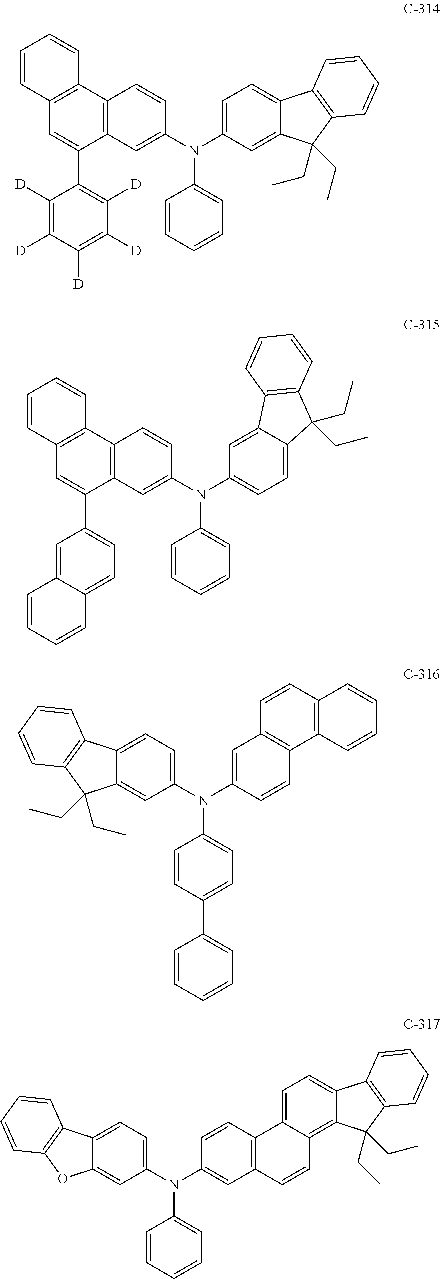 [SMILES: [2H]c1c([2H])c([2H])c(-c2cc3ccccc3c3ccc(N(c4ccccc4)c4ccc5c(c4)C(CC)(CC)c4ccccc4-5)cc23)c([2H])c1[2H] ; CCC1(CC)c2ccccc2-c2cc(N(c3ccccc3)c3ccc4c(c3)c(-c3ccc5ccccc5c3)cc3ccccc34)ccc21 ; CCC1(CC)c2ccccc2-c2ccc(N(c3ccc(-c4ccccc4)cc3)c3ccc4c(ccc5ccccc54)c3)cc21 ; CCC1(CC)c2ccccc2-c2ccc3c(ccc4cc(N(c5ccccc5)c5ccc6c(c5)oc5ccccc56)ccc43)c21] 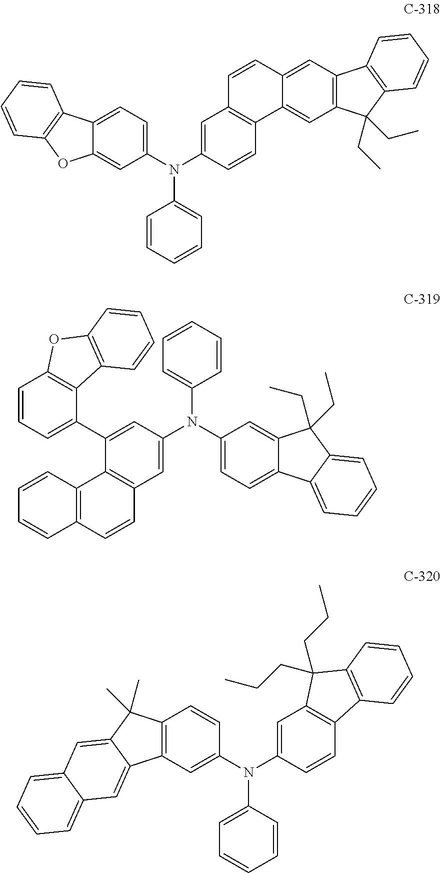 [SMILES: CCC1(CC)c2ccccc2-c2cc3ccc4cc(N(c5ccccc5)c5ccc6c(c5)oc5ccccc56)ccc4c3cc21 ; CCC1(CC)c2ccccc2-c2ccc(N(c3ccccc3)c3cc(-c4cccc5oc6ccccc6c45)c4c(ccc5ccccc54)c3)cc21 ; CCCC1(CCC)c2ccccc2-c2ccc(N(c3ccccc3)c3ccc4c(c3)-c3cc5ccccc5cc3C4(C)C)cc21] 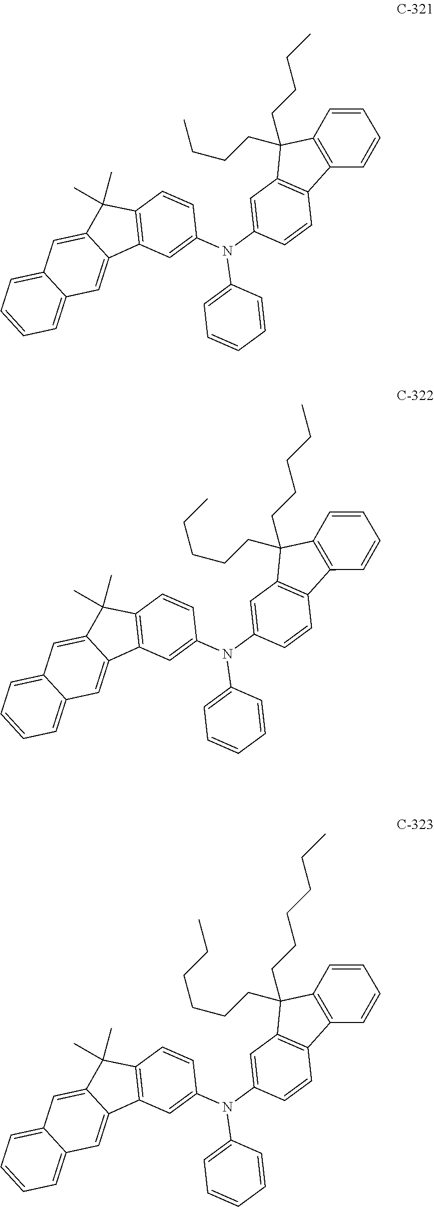 [SMILES: CCCCC1(CCCC)c2ccccc2-c2ccc(N(c3ccccc3)c3ccc4c(c3)-c3cc5ccccc5cc3C4(C)C)cc21 ; CCCCCC1(CCCCC)c2ccccc2-c2ccc(N(c3ccccc3)c3ccc4c(c3)-c3cc5ccccc5cc3C4(C)C)cc21 ; CCCCCCC1(CCCCCC)c2ccccc2-c2ccc(N(c3ccccc3)c3ccc4c(c3)-c3cc5ccccc5cc3C4(C)C)cc21] 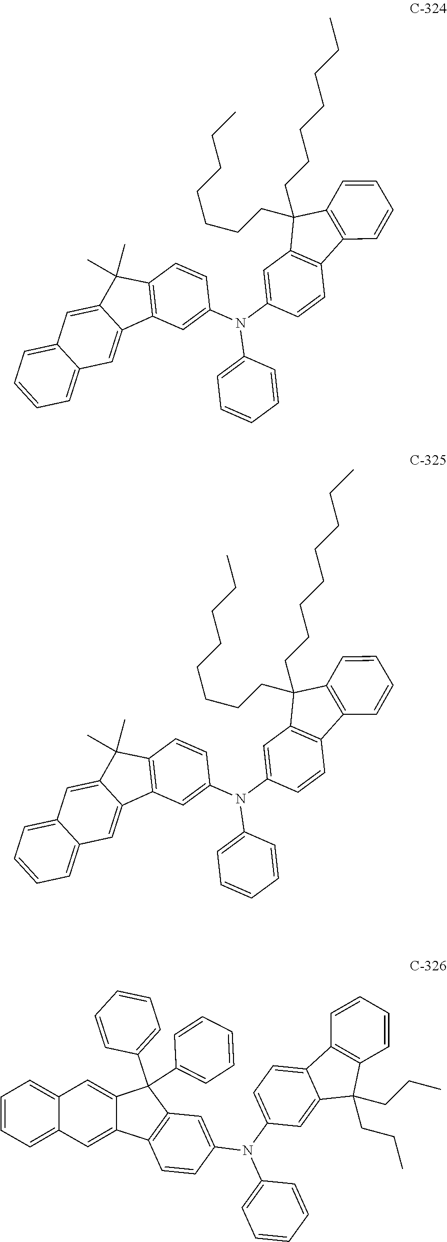 [SMILES: CCCCCCCC1(CCCCCCC)c2ccccc2-c2ccc(N(c3ccccc3)c3ccc4c(c3)-c3cc5ccccc5cc3C4(C)C)cc21 ; CCCCCCCCC1(CCCCCCCC)c2ccccc2-c2ccc(N(c3ccccc3)c3ccc4c(c3)-c3cc5ccccc5cc3C4(C)C)cc21 ; CCCC1(CCC)c2ccccc2-c2ccc(N(c3ccccc3)c3ccc4c(c3)C(c3ccccc3)(c3ccccc3)c3cc5ccccc5cc3-4)cc21] 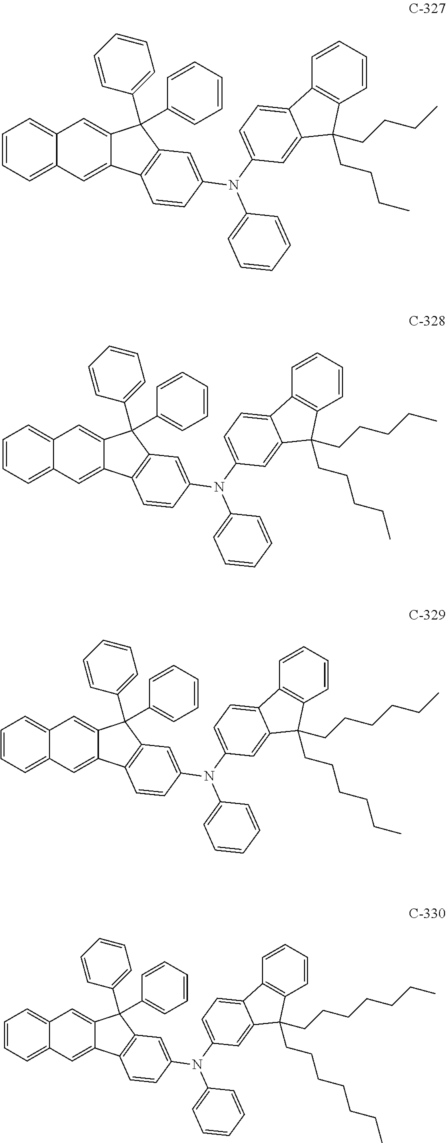 [SMILES: CCCCC1(CCCC)c2ccccc2-c2ccc(N(c3ccccc3)c3ccc4c(c3)C(c3ccccc3)(c3ccccc3)c3cc5ccccc5cc3-4)cc21 ; CCCCCC1(CCCCC)c2ccccc2-c2ccc(N(c3ccccc3)c3ccc4c(c3)C(c3ccccc3)(c3ccccc3)c3cc5ccccc5cc3-4)cc21 ; CCCCCCC1(CCCCCC)c2ccccc2-c2ccc(N(c3ccccc3)c3ccc4c(c3)C(c3ccccc3)(c3ccccc3)c3cc5ccccc5cc3-4)cc21 ; CCCCCCCC1(CCCCCCC)c2ccccc2-c2ccc(N(c3ccccc3)c3ccc4c(c3)C(c3ccccc3)(c3ccccc3)c3cc5ccccc5cc3-4)cc21] 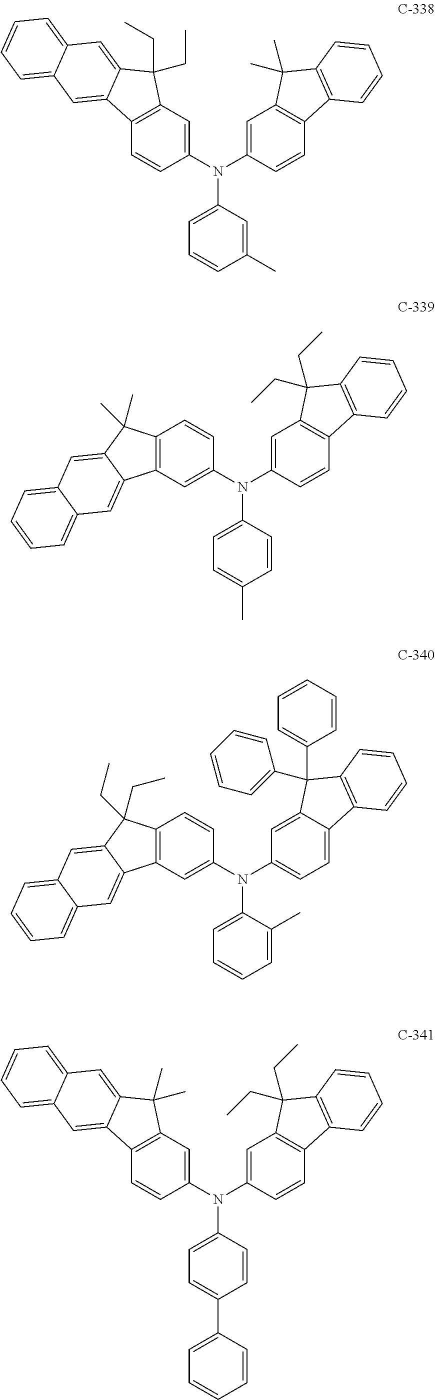 [SMILES: CCC1(CC)c2cc(N(c3cccc(C)c3)c3ccc4c(c3)C(C)(C)c3ccccc3-4)ccc2-c2cc3ccccc3cc21 ; CCC1(CC)c2ccccc2-c2ccc(N(c3ccc(C)cc3)c3ccc4c(c3)-c3cc5ccccc5cc3C4(C)C)cc21 ; CCC1(CC)c2ccc(N(c3ccc4c(c3)C(c3ccccc3)(c3ccccc3)c3ccccc3-4)c3ccccc3C)cc2-c2cc3ccccc3cc21 ; CCC1(CC)c2ccccc2-c2ccc(N(c3ccc(-c4ccccc4)cc3)c3ccc4c(c3)C(C)(C)c3cc5ccccc5cc3-4)cc21] 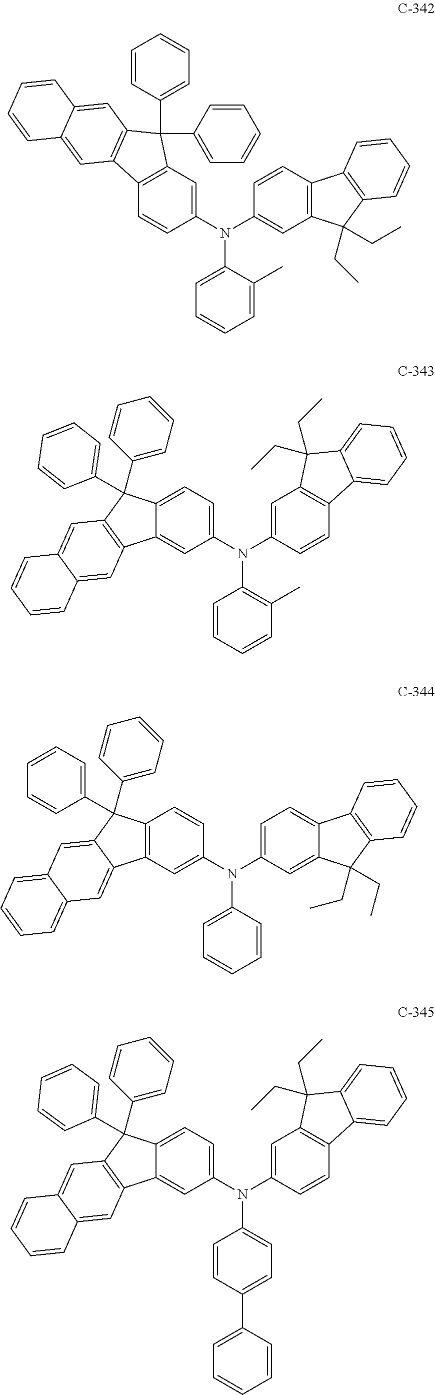 [SMILES: CCC1(CC)c2ccccc2-c2ccc(N(c3ccc4c(c3)C(c3ccccc3)(c3ccccc3)c3cc5ccccc5cc3-4)c3ccccc3C)cc21 ; CCC1(CC)c2ccccc2-c2ccc(N(c3ccc4c(c3)-c3cc5ccccc5cc3C4(c3ccccc3)c3ccccc3)c3ccccc3C)cc21 ; CCC1(CC)c2ccccc2-c2ccc(N(c3ccccc3)c3ccc4c(c3)-c3cc5ccccc5cc3C4(c3ccccc3)c3ccccc3)cc21 ; CCC1(CC)c2ccccc2-c2ccc(N(c3ccc(-c4ccccc4)cc3)c3ccc4c(c3)-c3cc5ccccc5cc3C4(c3ccccc3)c3ccccc3)cc21] 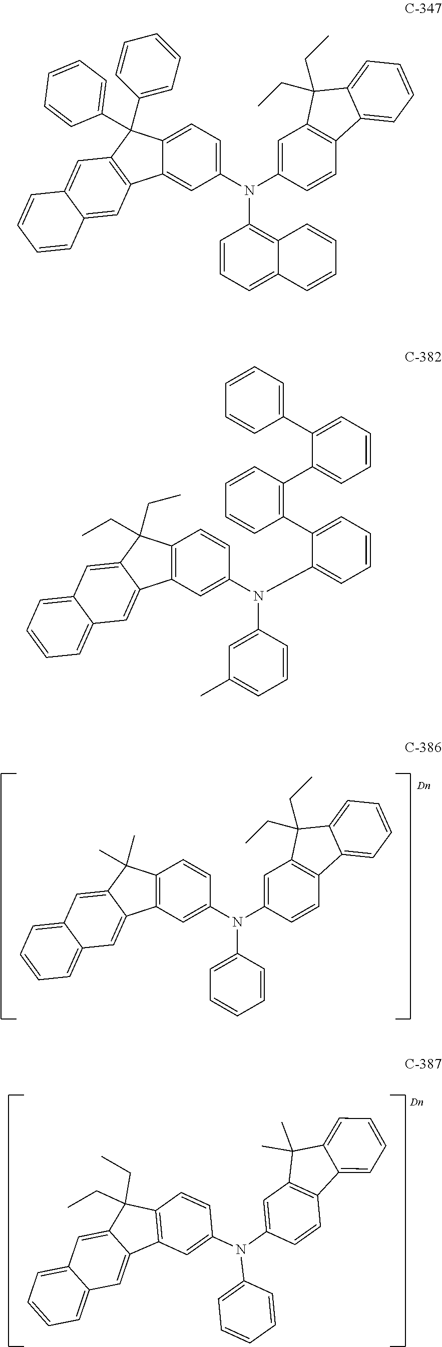 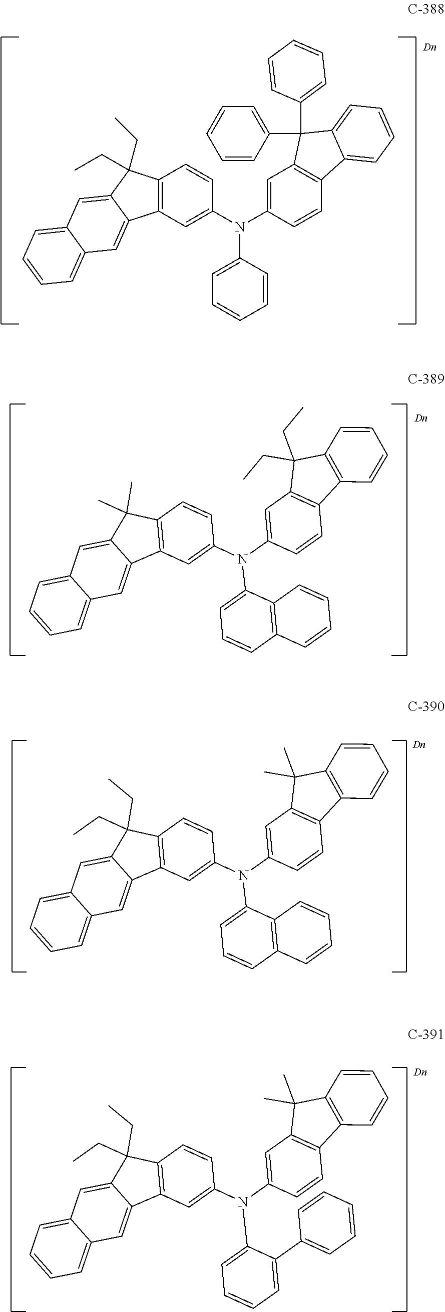 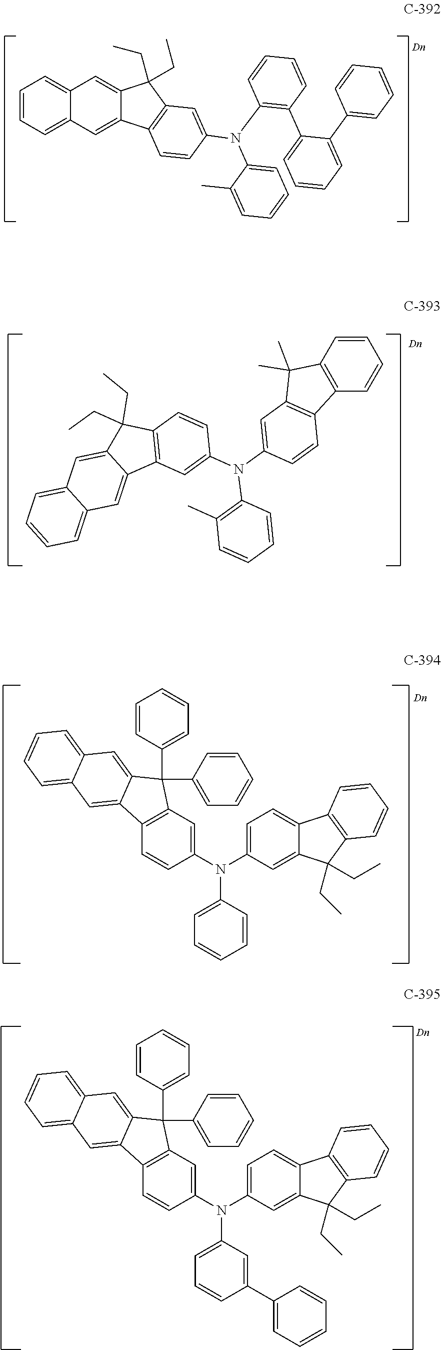 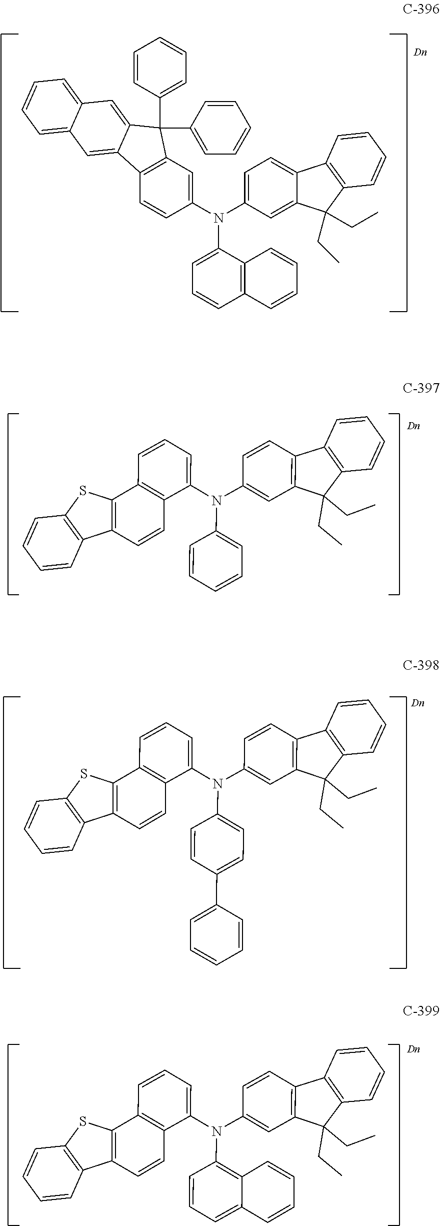 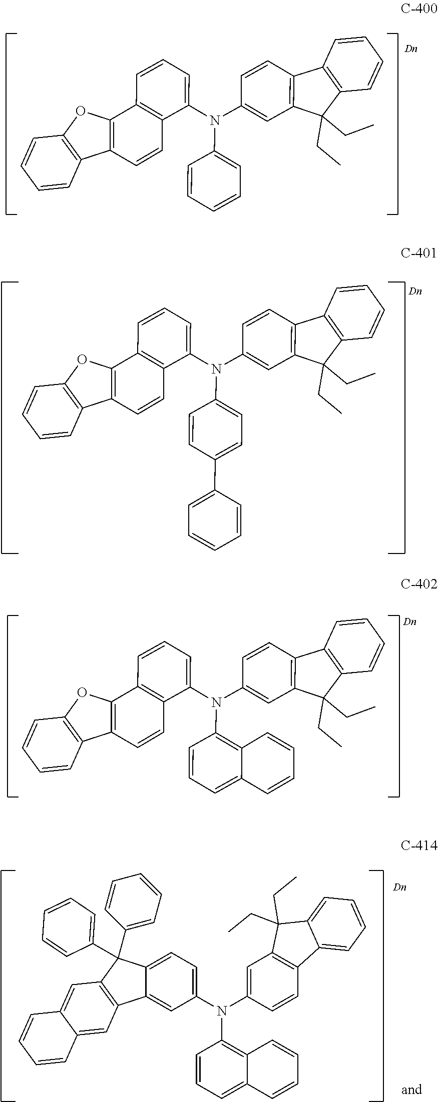 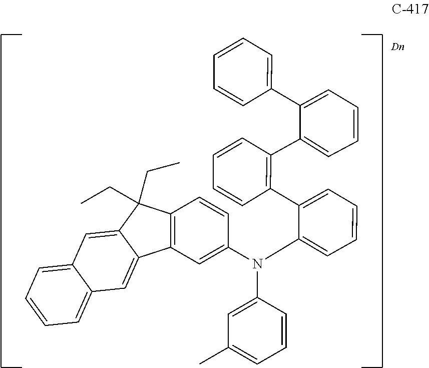
wherein in the compounds above, Dn represents that n number of hydrogens are replaced with deuterium, and n represents an integer of 1 or more, which is an integer of 1 to the maximum number of hydrogens in the compound.

16. The organic electroluminescent device comprising the organic electroluminescent compound according to claim 12.

17. The organic electroluminescent compound represented by the following Formula 3: 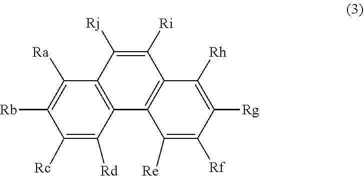
wherein in Formula 3,
at least one of Rₐ to Rⱼ is represented by the following Formula 3-1; 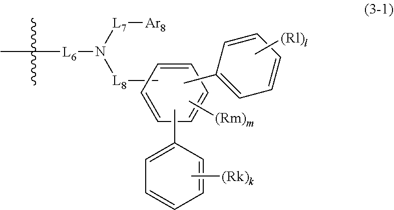
wherein in Formula 3 and Formula 3-1,
Rₐ to Rₘ each independently represent hydrogen, deuterium, a halogen, a cyano, a substituted or unsubstituted (C1-C30)alkyl, a substituted or unsubstituted (C3-C30)cycloalkyl, a substituted or unsubstituted (C6-C30)aryl, or a substituted or unsubstituted (3- to 30-membered)heteroaryl; [symbol]
represents the bond between Formula 3 and Formula 3-1;
L₆ to L₈ each independently represent a single bond, a substituted or unsubstituted (C6-C30)arylene, or a substituted or unsubstituted (3- to 30-membered)heteroarylene;
Ar₈ represents a substituted or unsubstituted (C6-C30)aryl, a substituted or unsubstituted (C3-C30)cycloalkyl, or a substituted or unsubstituted (3- to 30-membered)heteroaryl;
k and l each independently represent an integer of 1 to 5, m represents an integer of 1 to 3; and
if k to m represent an integer of 2 or more, each of Rₖ to Rₘ may be the same as or different from each other.

18. The organic electroluminescent compound according to claim 17, wherein the compound represented by Formula 3-1 is represented by any one of the following Formulas 3-1a to 3-1f: 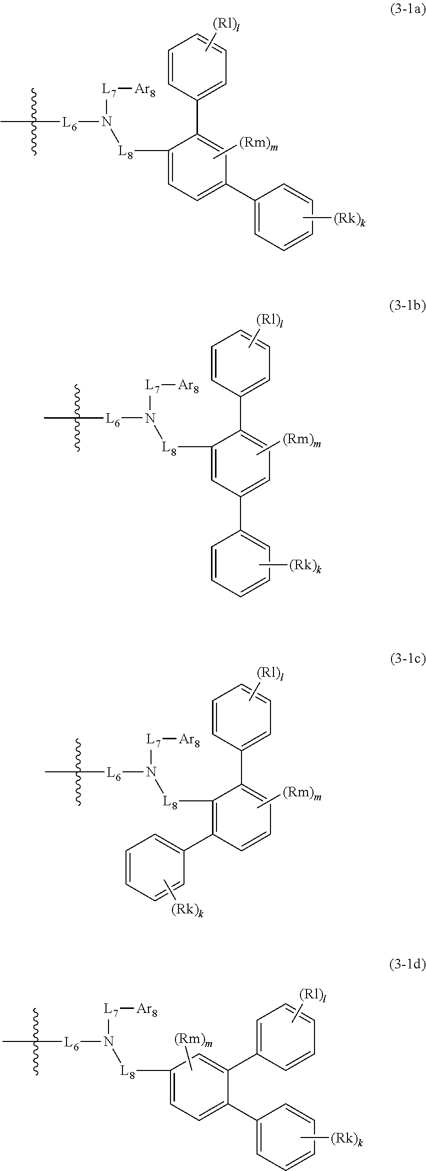 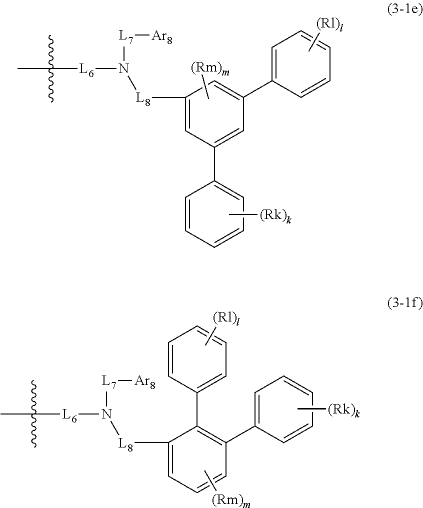
wherein in Formulas 3-1a to 3-1f, [symbol]
represents a linking group connected to Formula 3;
L₆ to L₈, Ar₈, and Rₖ to Rₘ are as defined in claim 17;
k and l each independently represent an integer of 1 to 5, and m represents an integer of 1 to 3; and
if k to m represent an integer of 2 or more, each of Rₖ to Rₘ may be the same or different from each other.

19. The organic electroluminescent compound according to claim 17, wherein the compound represented by Formula 3 is at least one selected from the following compounds: 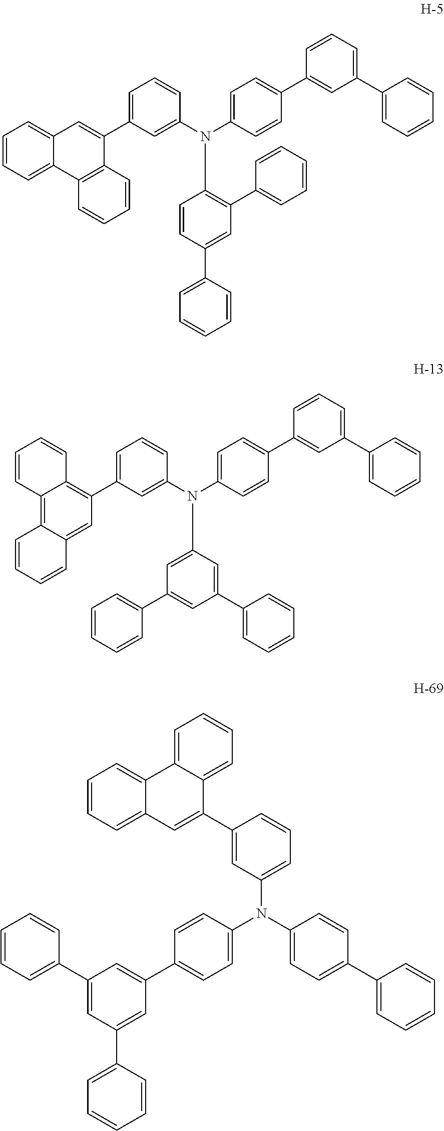 [SMILES: c1ccc(-c2cccc(-c3ccc(N(c4cccc(-c5cc6ccccc6c6ccccc56)c4)c4ccc(-c5ccccc5)cc4-c4ccccc4)cc3)c2)cc1 ; c1ccc(-c2cccc(-c3ccc(N(c4cc(-c5ccccc5)cc(-c5ccccc5)c4)c4cccc(-c5cc6ccccc6c6ccccc56)c4)cc3)c2)cc1 ; c1ccc(-c2ccc(N(c3ccc(-c4cc(-c5ccccc5)cc(-c5ccccc5)c4)cc3)c3cccc(-c4cc5ccccc5c5ccccc45)c3)cc2)cc1] 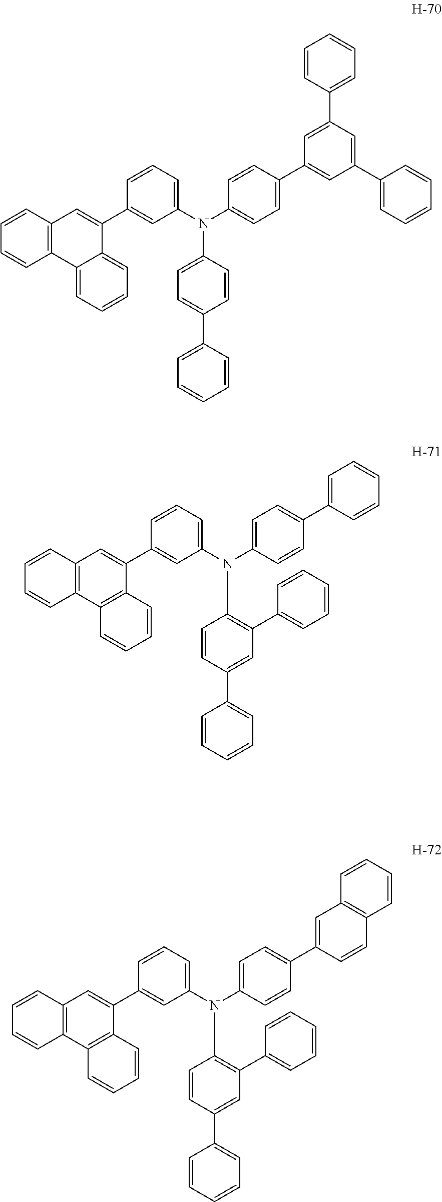 [SMILES: c1ccc(-c2ccc(N(c3ccc(-c4cc(-c5ccccc5)cc(-c5ccccc5)c4)cc3)c3cccc(-c4cc5ccccc5c5ccccc45)c3)cc2)cc1 ; c1ccc(-c2ccc(N(c3cccc(-c4cc5ccccc5c5ccccc45)c3)c3ccc(-c4ccccc4)cc3-c3ccccc3)cc2)cc1 ; c1ccc(-c2ccc(N(c3ccc(-c4ccc5ccccc5c4)cc3)c3cccc(-c4cc5ccccc5c5ccccc45)c3)c(-c3ccccc3)c2)cc1] 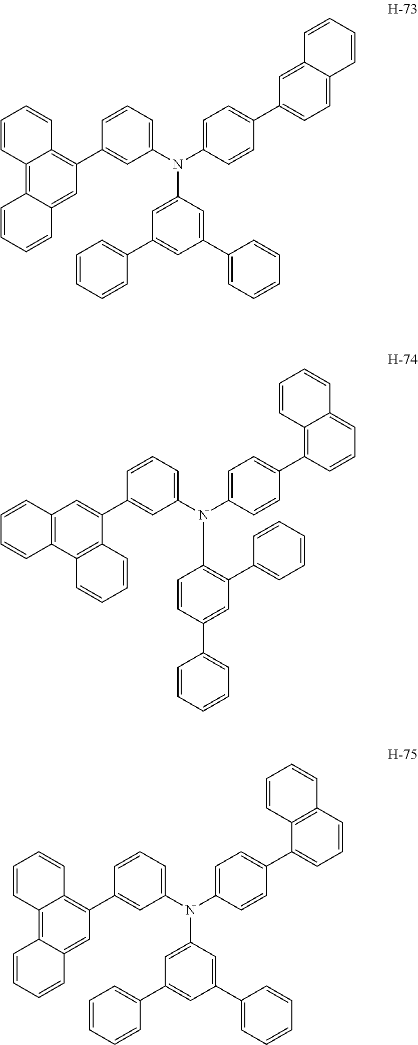 [SMILES: c1ccc(-c2cc(-c3ccccc3)cc(N(c3ccc(-c4ccc5ccccc5c4)cc3)c3cccc(-c4cc5ccccc5c5ccccc45)c3)c2)cc1 ; c1ccc(-c2ccc(N(c3ccc(-c4cccc5ccccc45)cc3)c3cccc(-c4cc5ccccc5c5ccccc45)c3)c(-c3ccccc3)c2)cc1 ; c1ccc(-c2cc(-c3ccccc3)cc(N(c3ccc(-c4cccc5ccccc45)cc3)c3cccc(-c4cc5ccccc5c5ccccc45)c3)c2)cc1] 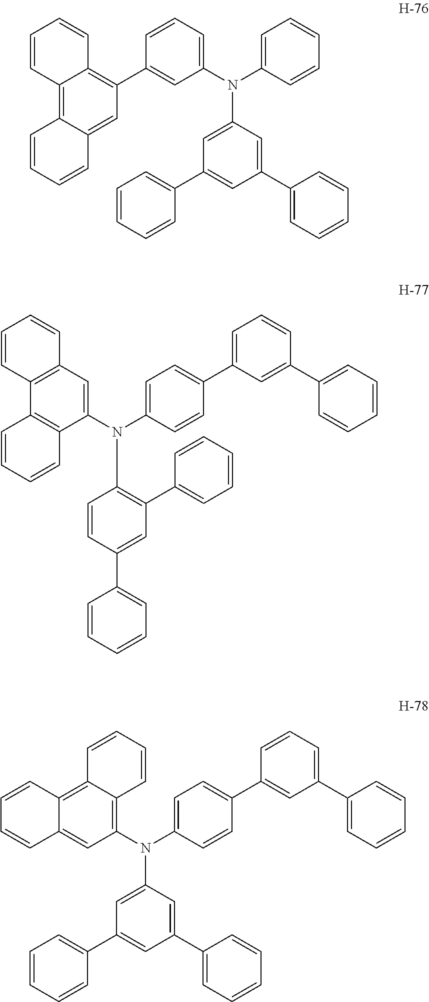 [SMILES: c1ccc(-c2cc(-c3ccccc3)cc(N(c3ccccc3)c3cccc(-c4cc5ccccc5c5ccccc45)c3)c2)cc1 ; c1ccc(-c2cccc(-c3ccc(N(c4ccc(-c5ccccc5)cc4-c4ccccc4)c4cc5ccccc5c5ccccc45)cc3)c2)cc1 ; c1ccc(-c2cccc(-c3ccc(N(c4cc(-c5ccccc5)cc(-c5ccccc5)c4)c4cc5ccccc5c5ccccc45)cc3)c2)cc1] 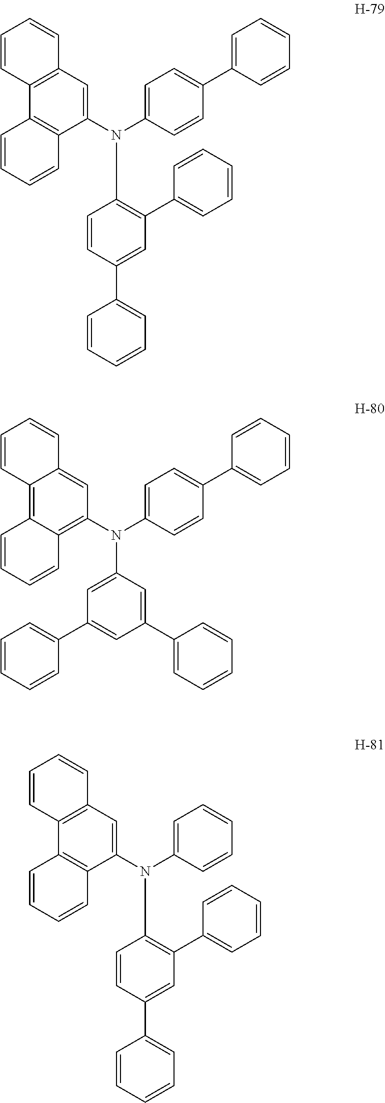 [SMILES: c1ccc(-c2ccc(N(c3ccc(-c4ccccc4)cc3-c3ccccc3)c3cc4ccccc4c4ccccc34)cc2)cc1 ; c1ccc(-c2ccc(N(c3cc(-c4ccccc4)cc(-c4ccccc4)c3)c3cc4ccccc4c4ccccc34)cc2)cc1 ; c1ccc(-c2ccc(N(c3ccccc3)c3cc4ccccc4c4ccccc34)c(-c3ccccc3)c2)cc1] 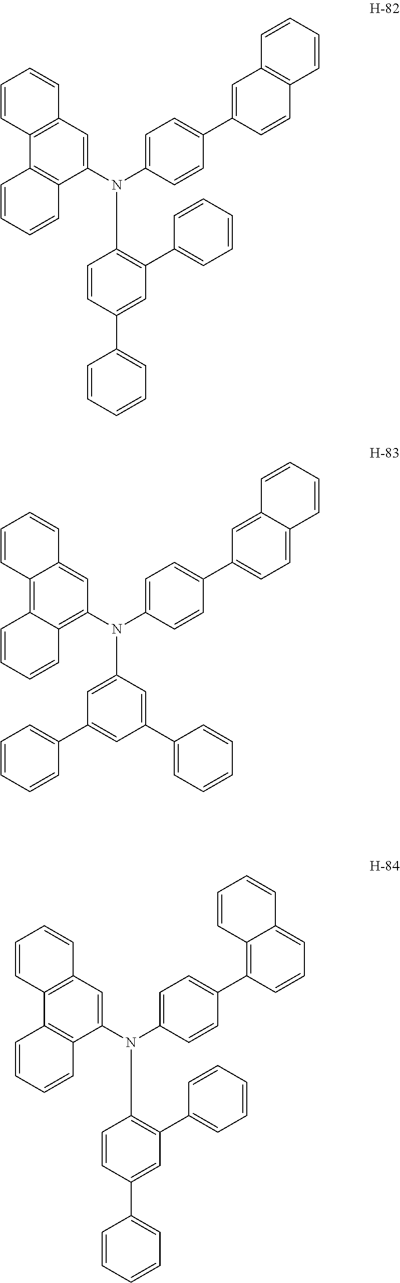 [SMILES: c1ccc(-c2ccc(N(c3ccc(-c4ccc5ccccc5c4)cc3)c3cc4ccccc4c4ccccc34)c(-c3ccccc3)c2)cc1 ; c1ccc(-c2cc(-c3ccccc3)cc(N(c3ccc(-c4ccc5ccccc5c4)cc3)c3cc4ccccc4c4ccccc34)c2)cc1 ; c1ccc(-c2ccc(N(c3ccc(-c4cccc5ccccc45)cc3)c3cc4ccccc4c4ccccc34)c(-c3ccccc3)c2)cc1] 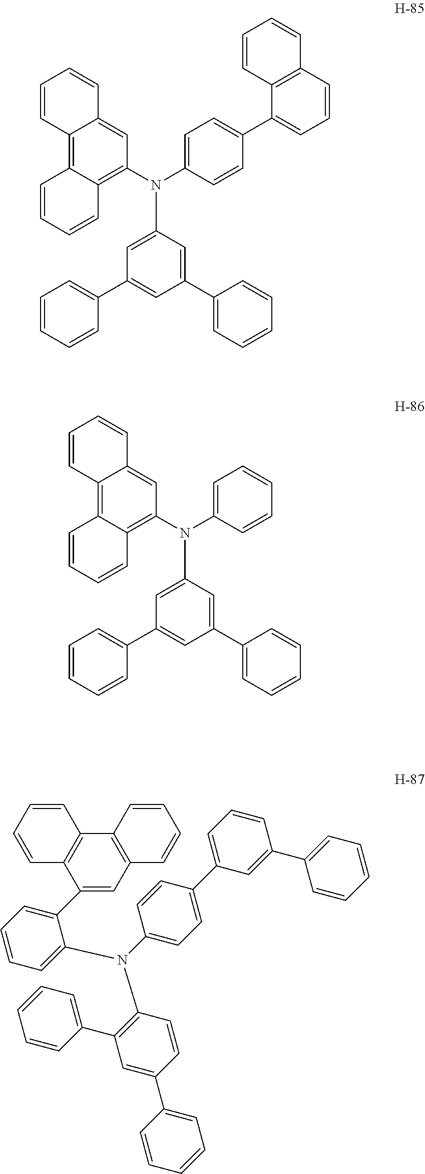 [SMILES: c1ccc(-c2cc(-c3ccccc3)cc(N(c3ccc(-c4cccc5ccccc45)cc3)c3cc4ccccc4c4ccccc34)c2)cc1 ; c1ccc(-c2cc(-c3ccccc3)cc(N(c3ccccc3)c3cc4ccccc4c4ccccc34)c2)cc1 ; c1ccc(-c2cccc(-c3ccc(N(c4ccc(-c5ccccc5)cc4-c4ccccc4)c4ccccc4-c4cc5ccccc5c5ccccc45)cc3)c2)cc1] 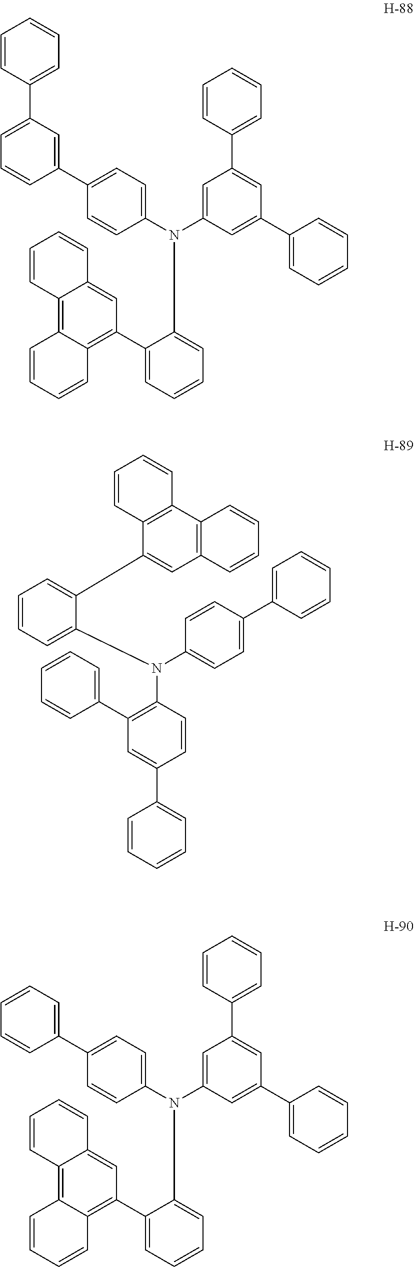 [SMILES: c1ccc(-c2cccc(-c3ccc(N(c4cc(-c5ccccc5)cc(-c5ccccc5)c4)c4ccccc4-c4cc5ccccc5c5ccccc45)cc3)c2)cc1 ; c1ccc(-c2ccc(N(c3ccc(-c4ccccc4)cc3-c3ccccc3)c3ccccc3-c3cc4ccccc4c4ccccc34)cc2)cc1 ; c1ccc(-c2ccc(N(c3cc(-c4ccccc4)cc(-c4ccccc4)c3)c3ccccc3-c3cc4ccccc4c4ccccc34)cc2)cc1] 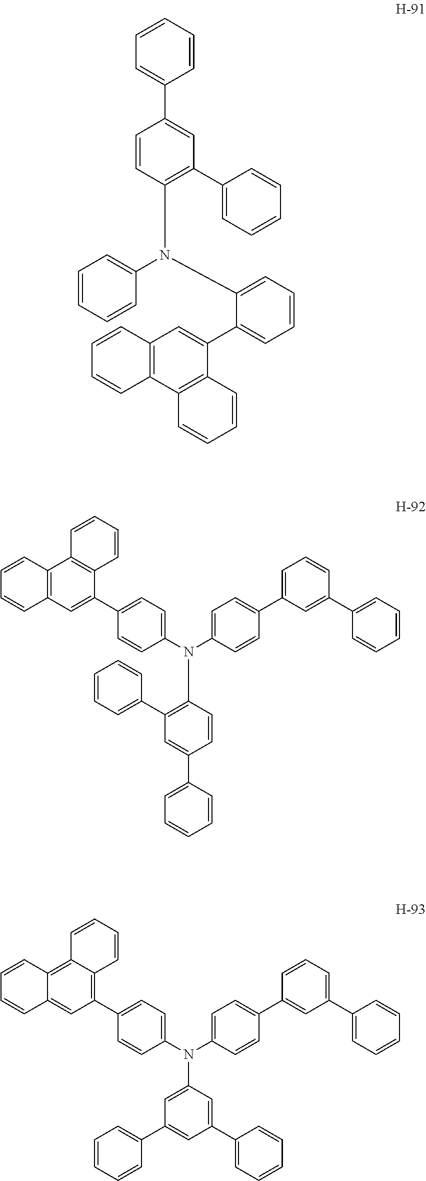 [SMILES: c1ccc(-c2ccc(N(c3ccccc3)c3ccccc3-c3cc4ccccc4c4ccccc34)c(-c3ccccc3)c2)cc1 ; c1ccc(-c2cccc(-c3ccc(N(c4ccc(-c5cc6ccccc6c6ccccc56)cc4)c4ccc(-c5ccccc5)cc4-c4ccccc4)cc3)c2)cc1 ; c1ccc(-c2cccc(-c3ccc(N(c4ccc(-c5cc6ccccc6c6ccccc56)cc4)c4cc(-c5ccccc5)cc(-c5ccccc5)c4)cc3)c2)cc1] 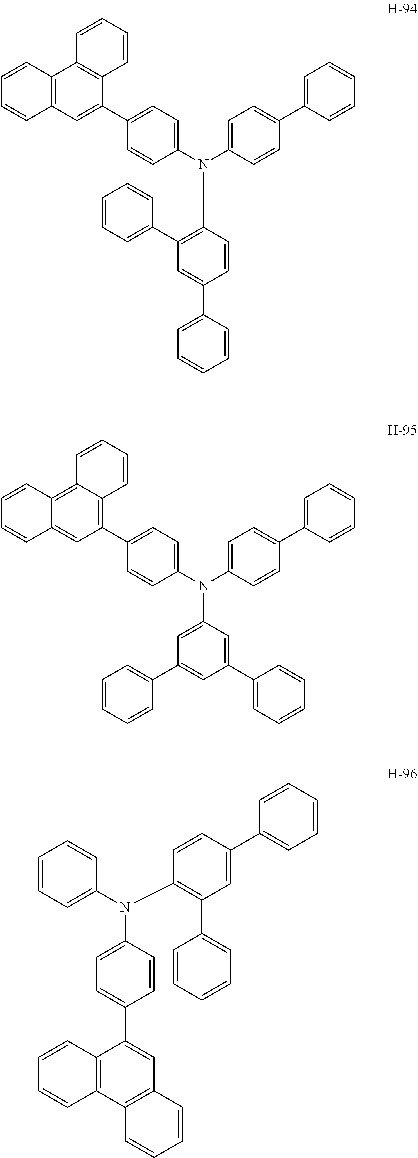 [SMILES: c1ccc(-c2ccc(N(c3ccc(-c4cc5ccccc5c5ccccc45)cc3)c3ccc(-c4ccccc4)cc3-c3ccccc3)cc2)cc1 ; c1ccc(-c2ccc(N(c3ccc(-c4cc5ccccc5c5ccccc45)cc3)c3cc(-c4ccccc4)cc(-c4ccccc4)c3)cc2)cc1 ; c1ccc(-c2ccc(N(c3ccccc3)c3ccc(-c4cc5ccccc5c5ccccc45)cc3)c(-c3ccccc3)c2)cc1] 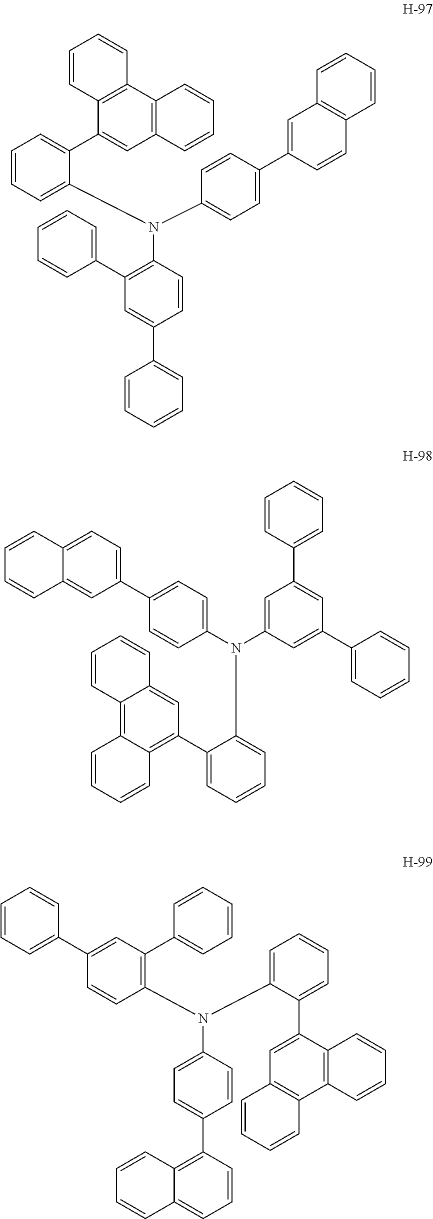 [SMILES: c1ccc(-c2ccc(N(c3ccc(-c4ccc5ccccc5c4)cc3)c3ccccc3-c3cc4ccccc4c4ccccc34)c(-c3ccccc3)c2)cc1 ; c1ccc(-c2cc(-c3ccccc3)cc(N(c3ccc(-c4ccc5ccccc5c4)cc3)c3ccccc3-c3cc4ccccc4c4ccccc34)c2)cc1 ; c1ccc(-c2ccc(N(c3ccc(-c4cccc5ccccc45)cc3)c3ccccc3-c3cc4ccccc4c4ccccc34)c(-c3ccccc3)c2)cc1] 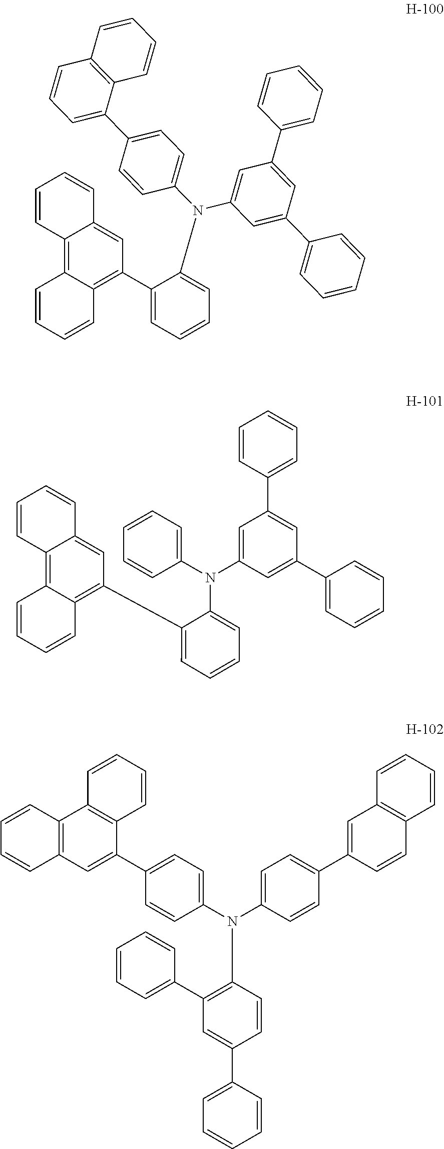 [SMILES: c1ccc(-c2cc(-c3ccccc3)cc(N(c3ccc(-c4cccc5ccccc45)cc3)c3ccccc3-c3cc4ccccc4c4ccccc34)c2)cc1 ; c1ccc(-c2cc(-c3ccccc3)cc(N(c3ccccc3)c3ccccc3-c3cc4ccccc4c4ccccc34)c2)cc1 ; c1ccc(-c2ccc(N(c3ccc(-c4ccc5ccccc5c4)cc3)c3ccc(-c4cc5ccccc5c5ccccc45)cc3)c(-c3ccccc3)c2)cc1] 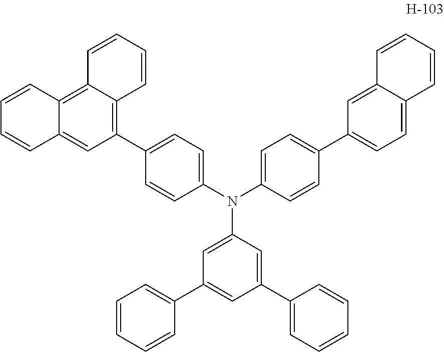 [SMILES: c1ccc(-c2cc(-c3ccccc3)cc(N(c3ccc(-c4ccc5ccccc5c4)cc3)c3ccc(-c4cc5ccccc5c5ccccc45)cc3)c2)cc1] 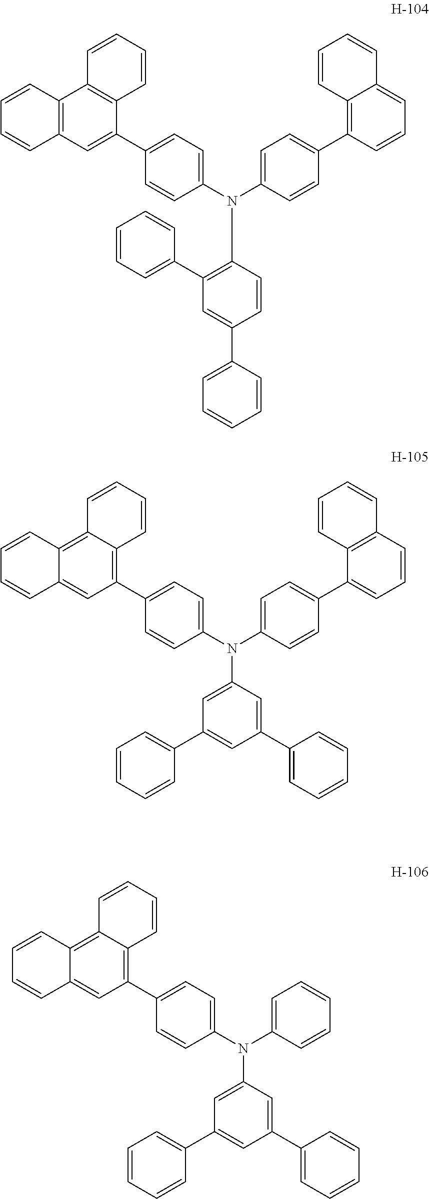 [SMILES: c1ccc(-c2ccc(N(c3ccc(-c4cccc5ccccc45)cc3)c3ccc(-c4cc5ccccc5c5ccccc45)cc3)c(-c3ccccc3)c2)cc1 ; c1ccc(-c2cc(-c3ccccc3)cc(N(c3ccc(-c4cccc5ccccc45)cc3)c3ccc(-c4cc5ccccc5c5ccccc45)cc3)c2)cc1 ; c1ccc(-c2cc(-c3ccccc3)cc(N(c3ccccc3)c3ccc(-c4cc5ccccc5c5ccccc45)cc3)c2)cc1] 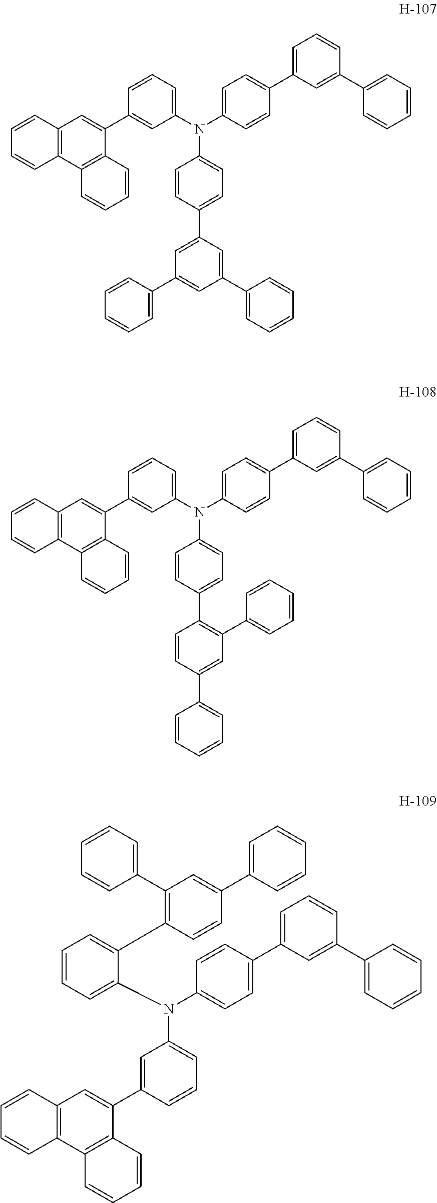 [SMILES: c1ccc(-c2cccc(-c3ccc(N(c4ccc(-c5cc(-c6ccccc6)cc(-c6ccccc6)c5)cc4)c4cccc(-c5cc6ccccc6c6ccccc56)c4)cc3)c2)cc1 ; c1ccc(-c2cccc(-c3ccc(N(c4ccc(-c5ccc(-c6ccccc6)cc5-c5ccccc5)cc4)c4cccc(-c5cc6ccccc6c6ccccc56)c4)cc3)c2)cc1 ; c1ccc(-c2cccc(-c3ccc(N(c4cccc(-c5cc6ccccc6c6ccccc56)c4)c4ccccc4-c4ccc(-c5ccccc5)cc4-c4ccccc4)cc3)c2)cc1] 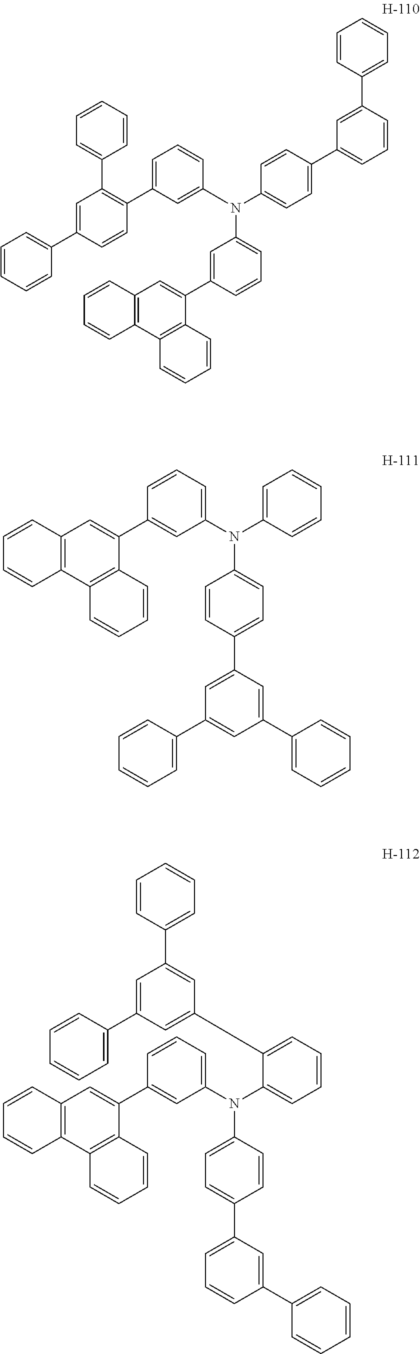 [SMILES: c1ccc(-c2cccc(-c3ccc(N(c4cccc(-c5ccc(-c6ccccc6)cc5-c5ccccc5)c4)c4cccc(-c5cc6ccccc6c6ccccc56)c4)cc3)c2)cc1 ; c1ccc(-c2cc(-c3ccccc3)cc(-c3ccc(N(c4ccccc4)c4cccc(-c5cc6ccccc6c6ccccc56)c4)cc3)c2)cc1 ; c1ccc(-c2cccc(-c3ccc(N(c4cccc(-c5cc6ccccc6c6ccccc56)c4)c4ccccc4-c4cc(-c5ccccc5)cc(-c5ccccc5)c4)cc3)c2)cc1] 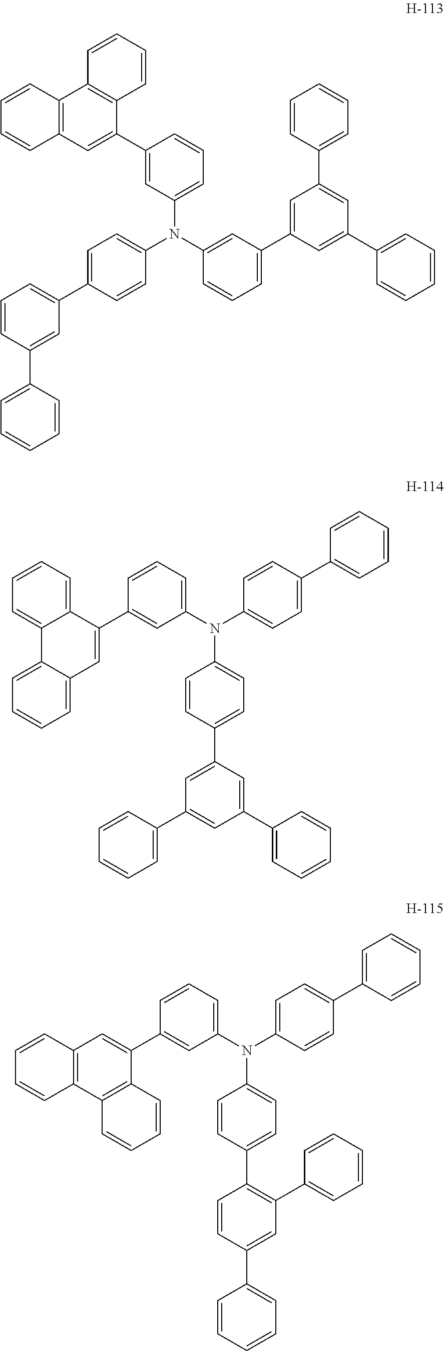 [SMILES: c1ccc(-c2cccc(-c3ccc(N(c4cccc(-c5cc(-c6ccccc6)cc(-c6ccccc6)c5)c4)c4cccc(-c5cc6ccccc6c6ccccc56)c4)cc3)c2)cc1 ; c1ccc(-c2ccc(N(c3ccc(-c4cc(-c5ccccc5)cc(-c5ccccc5)c4)cc3)c3cccc(-c4cc5ccccc5c5ccccc45)c3)cc2)cc1 ; c1ccc(-c2ccc(N(c3ccc(-c4ccc(-c5ccccc5)cc4-c4ccccc4)cc3)c3cccc(-c4cc5ccccc5c5ccccc45)c3)cc2)cc1] 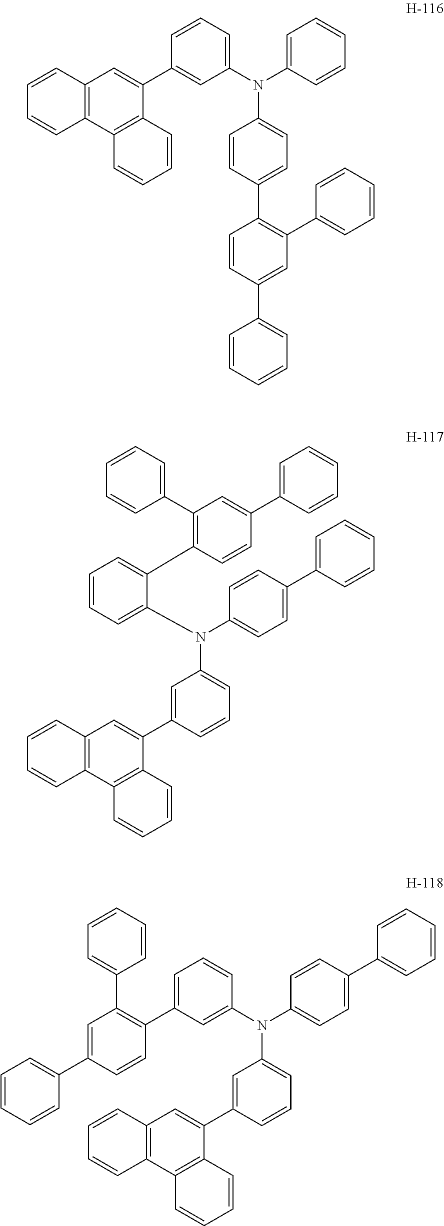 [SMILES: c1ccc(-c2ccc(-c3ccc(N(c4ccccc4)c4cccc(-c5cc6ccccc6c6ccccc56)c4)cc3)c(-c3ccccc3)c2)cc1 ; c1ccc(-c2ccc(N(c3cccc(-c4cc5ccccc5c5ccccc45)c3)c3ccccc3-c3ccc(-c4ccccc4)cc3-c3ccccc3)cc2)cc1 ; c1ccc(-c2ccc(N(c3cccc(-c4ccc(-c5ccccc5)cc4-c4ccccc4)c3)c3cccc(-c4cc5ccccc5c5ccccc45)c3)cc2)cc1] 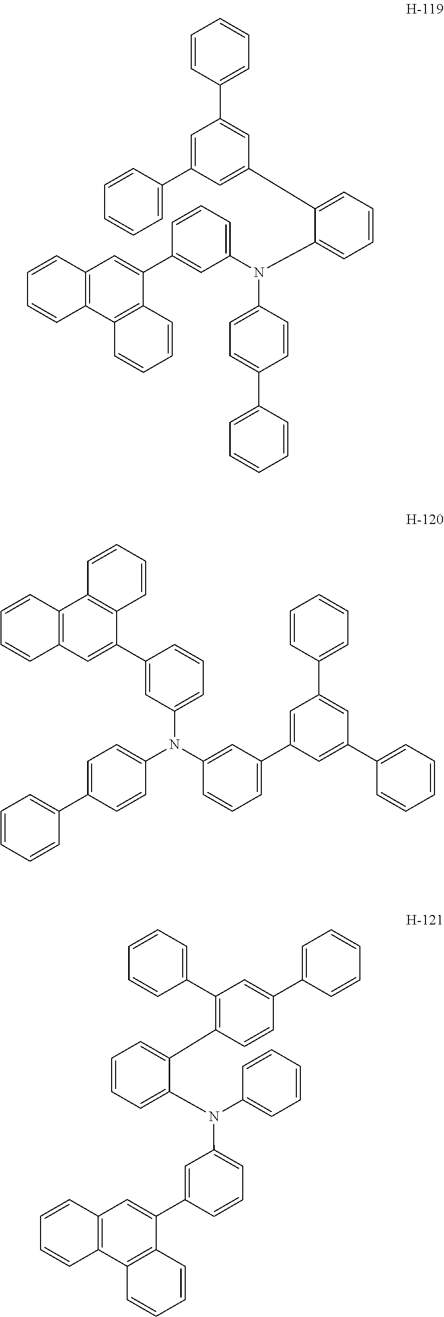 [SMILES: c1ccc(-c2ccc(N(c3cccc(-c4cc5ccccc5c5ccccc45)c3)c3ccccc3-c3cc(-c4ccccc4)cc(-c4ccccc4)c3)cc2)cc1 ; c1ccc(-c2ccc(N(c3cccc(-c4cc(-c5ccccc5)cc(-c5ccccc5)c4)c3)c3cccc(-c4cc5ccccc5c5ccccc45)c3)cc2)cc1 ; c1ccc(-c2ccc(-c3ccccc3N(c3ccccc3)c3cccc(-c4cc5ccccc5c5ccccc45)c3)c(-c3ccccc3)c2)cc1] 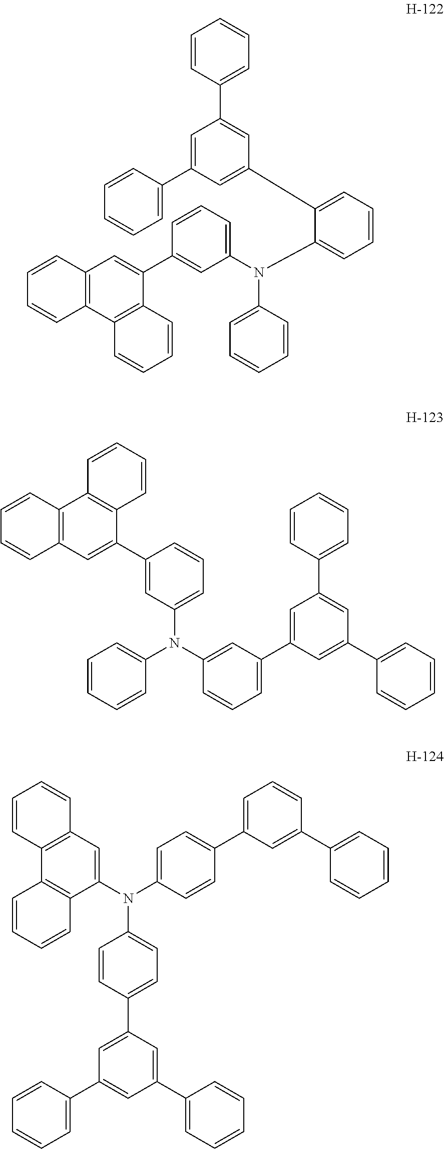 [SMILES: c1ccc(-c2cc(-c3ccccc3)cc(-c3ccccc3N(c3ccccc3)c3cccc(-c4cc5ccccc5c5ccccc45)c3)c2)cc1 ; c1ccc(-c2cc(-c3ccccc3)cc(-c3cccc(N(c4ccccc4)c4cccc(-c5cc6ccccc6c6ccccc56)c4)c3)c2)cc1 ; c1ccc(-c2cccc(-c3ccc(N(c4ccc(-c5cc(-c6ccccc6)cc(-c6ccccc6)c5)cc4)c4cc5ccccc5c5ccccc45)cc3)c2)cc1] 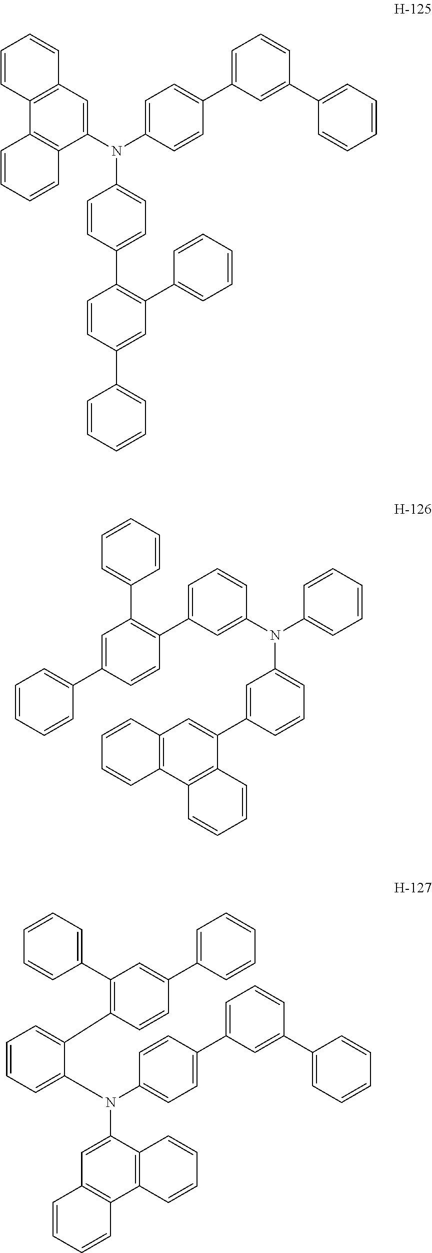 [SMILES: c1ccc(-c2cccc(-c3ccc(N(c4ccc(-c5ccc(-c6ccccc6)cc5-c5ccccc5)cc4)c4cc5ccccc5c5ccccc45)cc3)c2)cc1 ; c1ccc(-c2ccc(-c3cccc(N(c4ccccc4)c4cccc(-c5cc6ccccc6c6ccccc56)c4)c3)c(-c3ccccc3)c2)cc1 ; c1ccc(-c2cccc(-c3ccc(N(c4ccccc4-c4ccc(-c5ccccc5)cc4-c4ccccc4)c4cc5ccccc5c5ccccc45)cc3)c2)cc1] 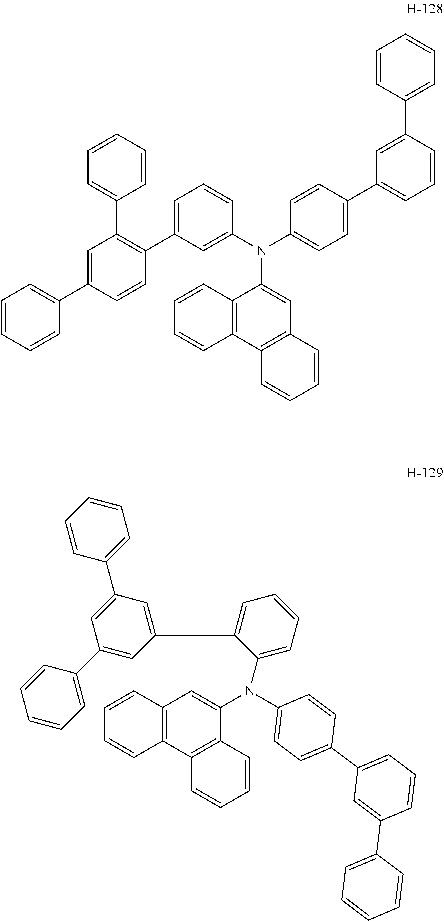 [SMILES: c1ccc(-c2cccc(-c3ccc(N(c4cccc(-c5ccc(-c6ccccc6)cc5-c5ccccc5)c4)c4cc5ccccc5c5ccccc45)cc3)c2)cc1 ; c1ccc(-c2cccc(-c3ccc(N(c4ccccc4-c4cc(-c5ccccc5)cc(-c5ccccc5)c4)c4cc5ccccc5c5ccccc45)cc3)c2)cc1] 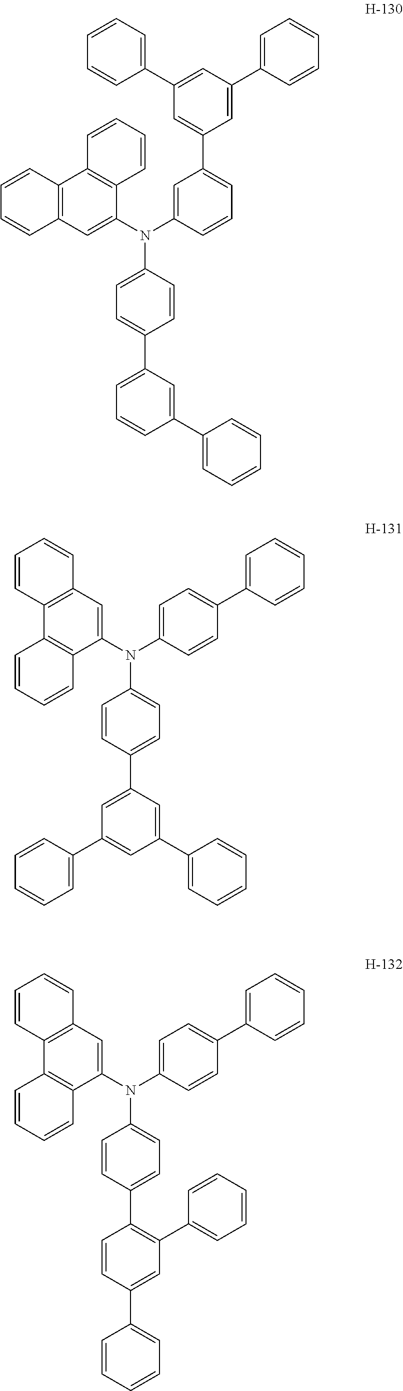 [SMILES: c1ccc(-c2cccc(-c3ccc(N(c4cccc(-c5cc(-c6ccccc6)cc(-c6ccccc6)c5)c4)c4cc5ccccc5c5ccccc45)cc3)c2)cc1 ; c1ccc(-c2ccc(N(c3ccc(-c4cc(-c5ccccc5)cc(-c5ccccc5)c4)cc3)c3cc4ccccc4c4ccccc34)cc2)cc1 ; c1ccc(-c2ccc(N(c3ccc(-c4ccc(-c5ccccc5)cc4-c4ccccc4)cc3)c3cc4ccccc4c4ccccc34)cc2)cc1] 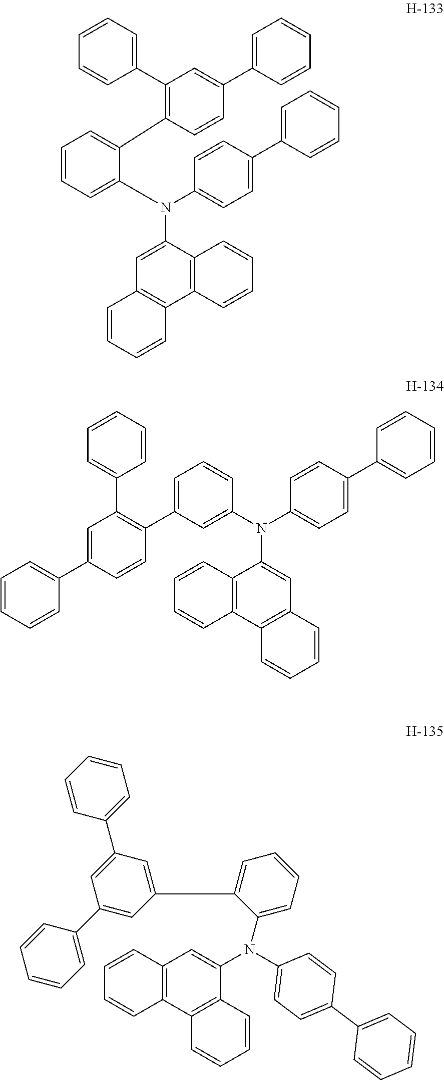 [SMILES: c1ccc(-c2ccc(N(c3ccccc3-c3ccc(-c4ccccc4)cc3-c3ccccc3)c3cc4ccccc4c4ccccc34)cc2)cc1 ; c1ccc(-c2ccc(N(c3cccc(-c4ccc(-c5ccccc5)cc4-c4ccccc4)c3)c3cc4ccccc4c4ccccc34)cc2)cc1 ; c1ccc(-c2ccc(N(c3ccccc3-c3cc(-c4ccccc4)cc(-c4ccccc4)c3)c3cc4ccccc4c4ccccc34)cc2)cc1] 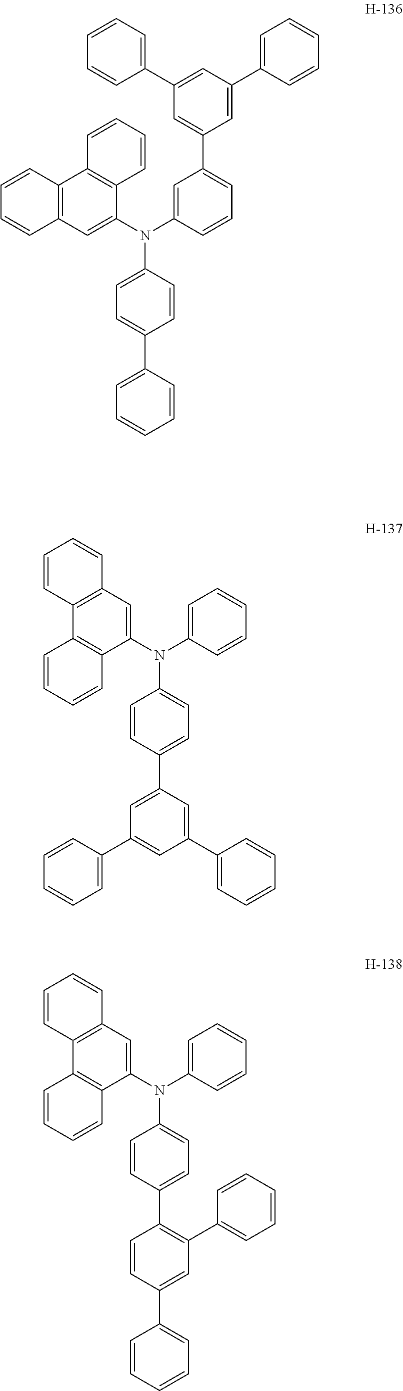 [SMILES: c1ccc(-c2ccc(N(c3cccc(-c4cc(-c5ccccc5)cc(-c5ccccc5)c4)c3)c3cc4ccccc4c4ccccc34)cc2)cc1 ; c1ccc(-c2cc(-c3ccccc3)cc(-c3ccc(N(c4ccccc4)c4cc5ccccc5c5ccccc45)cc3)c2)cc1 ; c1ccc(-c2ccc(-c3ccc(N(c4ccccc4)c4cc5ccccc5c5ccccc45)cc3)c(-c3ccccc3)c2)cc1] 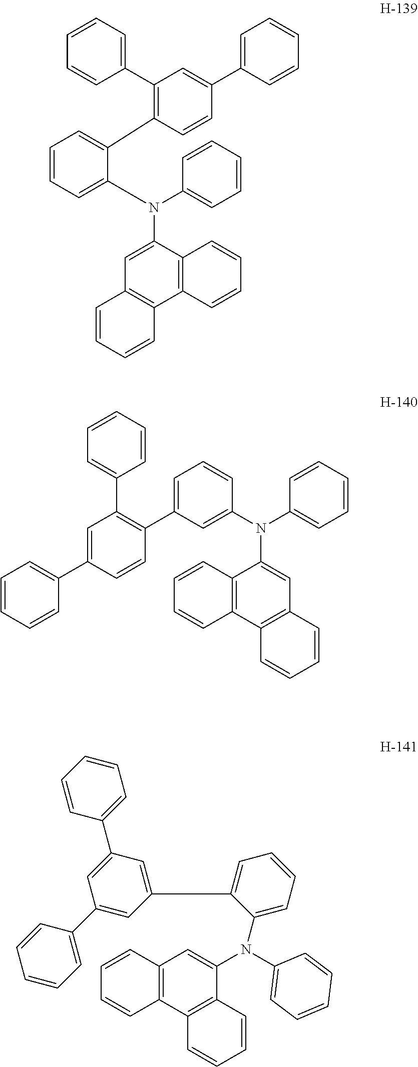 [SMILES: c1ccc(-c2ccc(-c3ccccc3N(c3ccccc3)c3cc4ccccc4c4ccccc34)c(-c3ccccc3)c2)cc1 ; c1ccc(-c2ccc(-c3cccc(N(c4ccccc4)c4cc5ccccc5c5ccccc45)c3)c(-c3ccccc3)c2)cc1 ; c1ccc(-c2cc(-c3ccccc3)cc(-c3ccccc3N(c3ccccc3)c3cc4ccccc4c4ccccc34)c2)cc1] 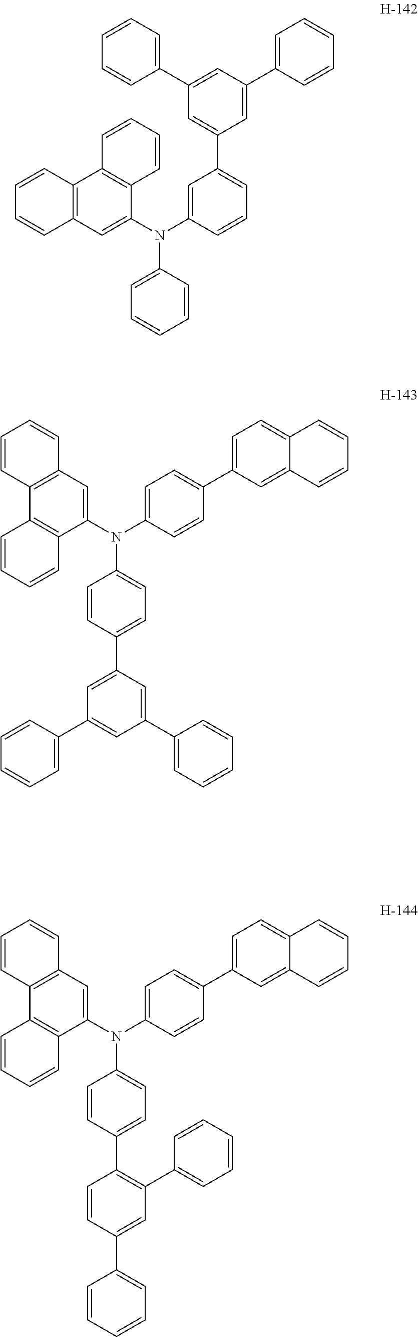 [SMILES: c1ccc(-c2cc(-c3ccccc3)cc(-c3cccc(N(c4ccccc4)c4cc5ccccc5c5ccccc45)c3)c2)cc1 ; c1ccc(-c2cc(-c3ccccc3)cc(-c3ccc(N(c4ccc(-c5ccc6ccccc6c5)cc4)c4cc5ccccc5c5ccccc45)cc3)c2)cc1 ; c1ccc(-c2ccc(-c3ccc(N(c4ccc(-c5ccc6ccccc6c5)cc4)c4cc5ccccc5c5ccccc45)cc3)c(-c3ccccc3)c2)cc1] 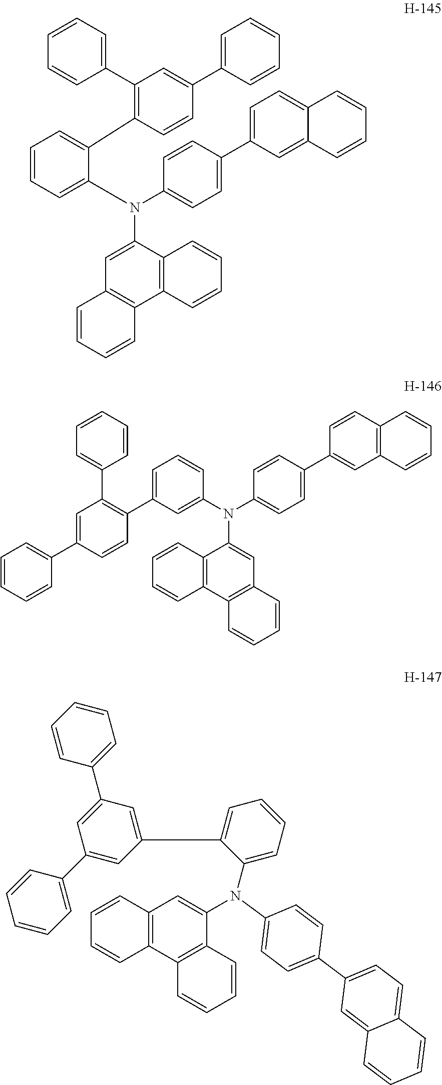 [SMILES: c1ccc(-c2ccc(-c3ccccc3N(c3ccc(-c4ccc5ccccc5c4)cc3)c3cc4ccccc4c4ccccc34)c(-c3ccccc3)c2)cc1 ; c1ccc(-c2ccc(-c3cccc(N(c4ccc(-c5ccc6ccccc6c5)cc4)c4cc5ccccc5c5ccccc45)c3)c(-c3ccccc3)c2)cc1 ; c1ccc(-c2cc(-c3ccccc3)cc(-c3ccccc3N(c3ccc(-c4ccc5ccccc5c4)cc3)c3cc4ccccc4c4ccccc34)c2)cc1] 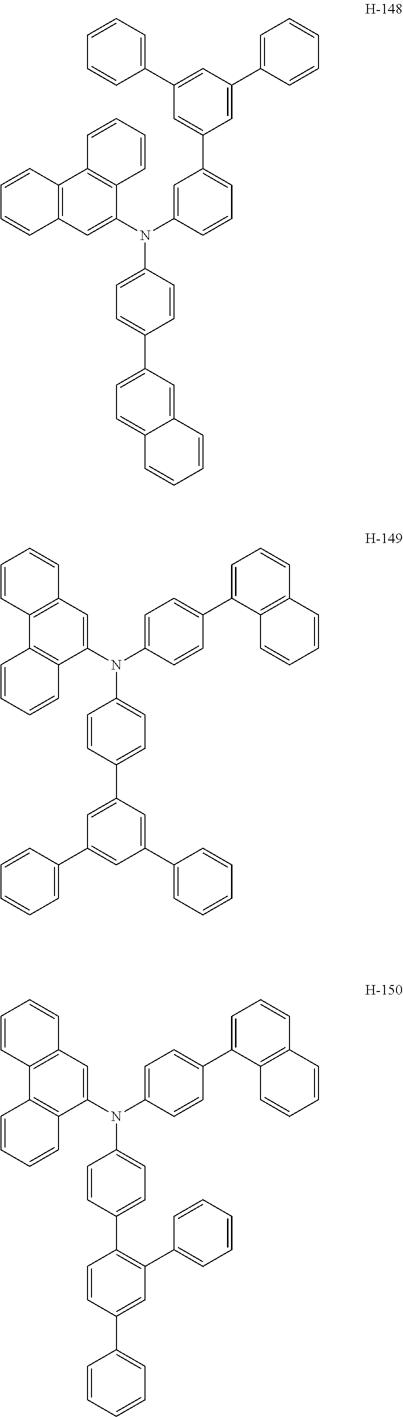 [SMILES: c1ccc(-c2cc(-c3ccccc3)cc(-c3cccc(N(c4ccc(-c5ccc6ccccc6c5)cc4)c4cc5ccccc5c5ccccc45)c3)c2)cc1 ; c1ccc(-c2cc(-c3ccccc3)cc(-c3ccc(N(c4ccc(-c5cccc6ccccc56)cc4)c4cc5ccccc5c5ccccc45)cc3)c2)cc1 ; c1ccc(-c2ccc(-c3ccc(N(c4ccc(-c5cccc6ccccc56)cc4)c4cc5ccccc5c5ccccc45)cc3)c(-c3ccccc3)c2)cc1] 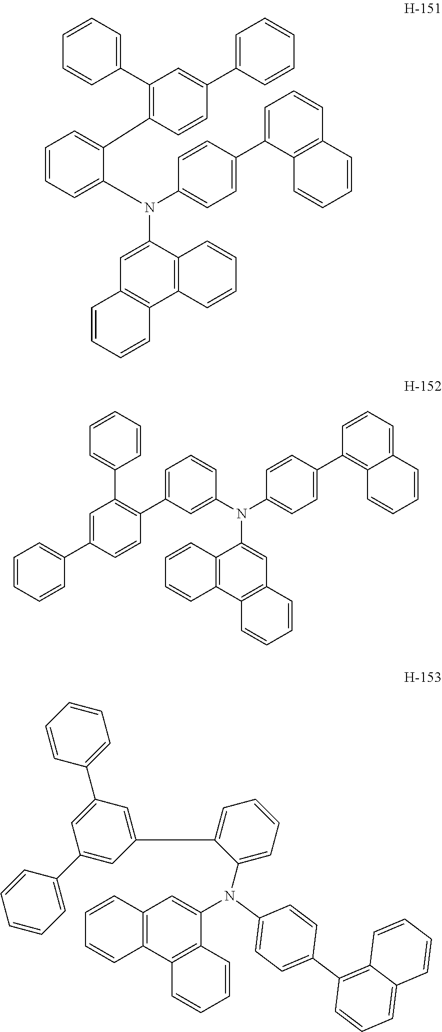 [SMILES: c1ccc(-c2ccc(-c3ccccc3N(c3ccc(-c4cccc5ccccc45)cc3)c3cc4ccccc4c4ccccc34)c(-c3ccccc3)c2)cc1 ; c1ccc(-c2ccc(-c3cccc(N(c4ccc(-c5cccc6ccccc56)cc4)c4cc5ccccc5c5ccccc45)c3)c(-c3ccccc3)c2)cc1 ; c1ccc(-c2cc(-c3ccccc3)cc(-c3ccccc3N(c3ccc(-c4cccc5ccccc45)cc3)c3cc4ccccc4c4ccccc34)c2)cc1] 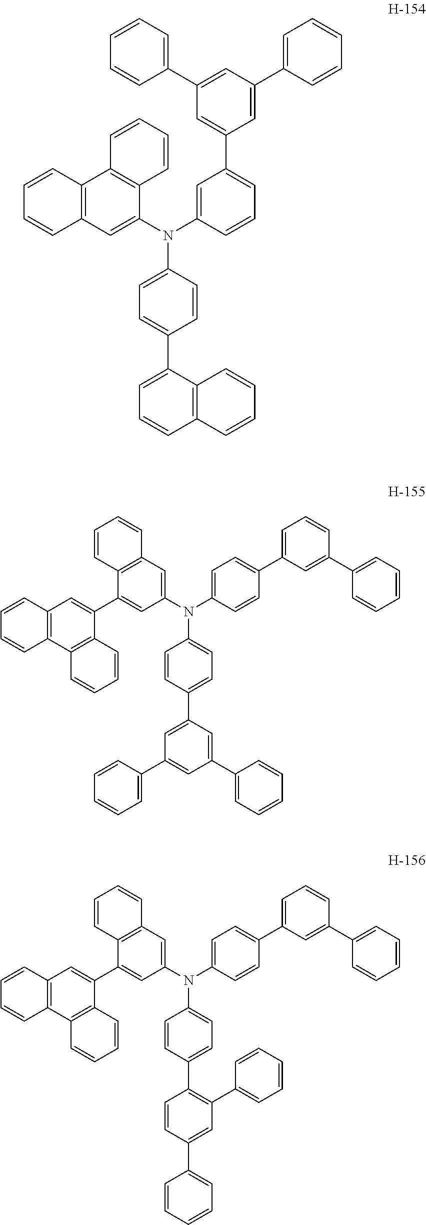 [SMILES: c1ccc(-c2cc(-c3ccccc3)cc(-c3cccc(N(c4ccc(-c5cccc6ccccc56)cc4)c4cc5ccccc5c5ccccc45)c3)c2)cc1 ; c1ccc(-c2cccc(-c3ccc(N(c4ccc(-c5cc(-c6ccccc6)cc(-c6ccccc6)c5)cc4)c4cc(-c5cc6ccccc6c6ccccc56)c5ccccc5c4)cc3)c2)cc1 ; c1ccc(-c2cccc(-c3ccc(N(c4ccc(-c5ccc(-c6ccccc6)cc5-c5ccccc5)cc4)c4cc(-c5cc6ccccc6c6ccccc56)c5ccccc5c4)cc3)c2)cc1] 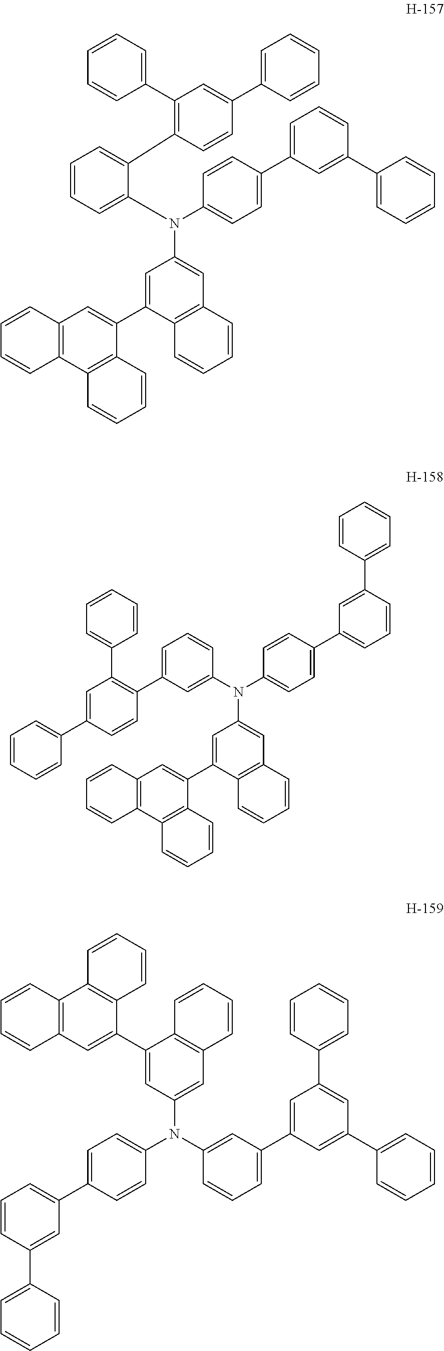 [SMILES: c1ccc(-c2cccc(-c3ccc(N(c4cc(-c5cc6ccccc6c6ccccc56)c5ccccc5c4)c4ccccc4-c4ccc(-c5ccccc5)cc4-c4ccccc4)cc3)c2)cc1 ; c1ccc(-c2cccc(-c3ccc(N(c4cccc(-c5ccc(-c6ccccc6)cc5-c5ccccc5)c4)c4cc(-c5cc6ccccc6c6ccccc56)c5ccccc5c4)cc3)c2)cc1 ; c1ccc(-c2cccc(-c3ccc(N(c4cccc(-c5cc(-c6ccccc6)cc(-c6ccccc6)c5)c4)c4cc(-c5cc6ccccc6c6ccccc56)c5ccccc5c4)cc3)c2)cc1] 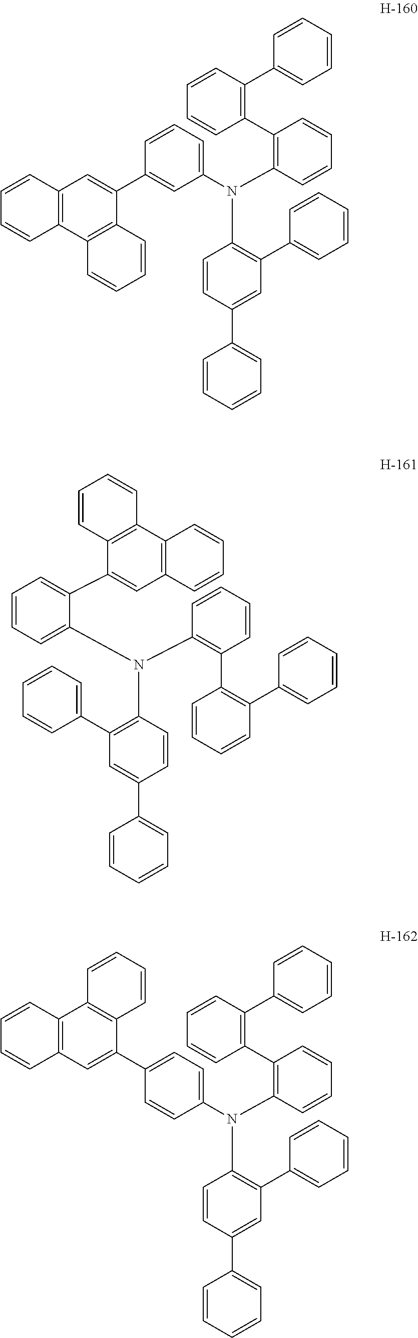 [SMILES: c1ccc(-c2ccc(N(c3cccc(-c4cc5ccccc5c5ccccc45)c3)c3ccccc3-c3ccccc3-c3ccccc3)c(-c3ccccc3)c2)cc1 ; c1ccc(-c2ccc(N(c3ccccc3-c3ccccc3-c3ccccc3)c3ccccc3-c3cc4ccccc4c4ccccc34)c(-c3ccccc3)c2)cc1 ; c1ccc(-c2ccc(N(c3ccc(-c4cc5ccccc5c5ccccc45)cc3)c3ccccc3-c3ccccc3-c3ccccc3)c(-c3ccccc3)c2)cc1] 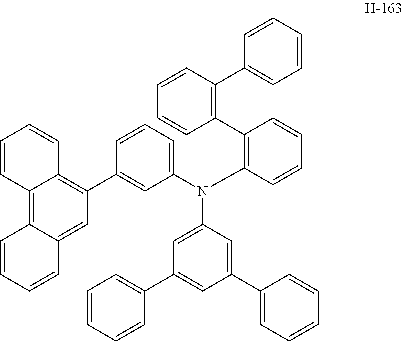 [SMILES: c1ccc(-c2cc(-c3ccccc3)cc(N(c3cccc(-c4cc5ccccc5c5ccccc45)c3)c3ccccc3-c3ccccc3-c3ccccc3)c2)cc1] 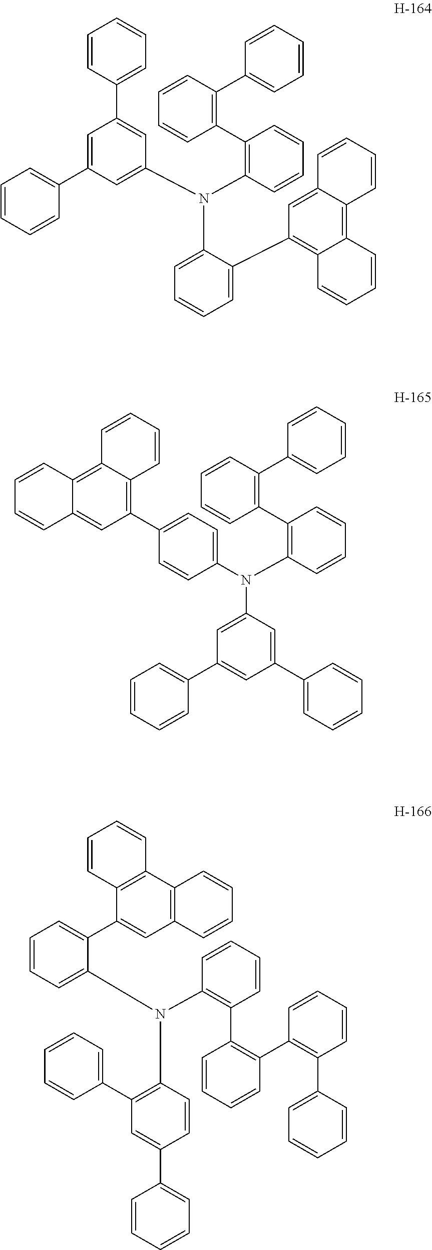 [SMILES: c1ccc(-c2cc(-c3ccccc3)cc(N(c3ccccc3-c3ccccc3-c3ccccc3)c3ccccc3-c3cc4ccccc4c4ccccc34)c2)cc1 ; c1ccc(-c2cc(-c3ccccc3)cc(N(c3ccc(-c4cc5ccccc5c5ccccc45)cc3)c3ccccc3-c3ccccc3-c3ccccc3)c2)cc1 ; c1ccc(-c2ccc(N(c3ccccc3-c3ccccc3-c3ccccc3-c3ccccc3)c3ccccc3-c3cc4ccccc4c4ccccc34)c(-c3ccccc3)c2)cc1] 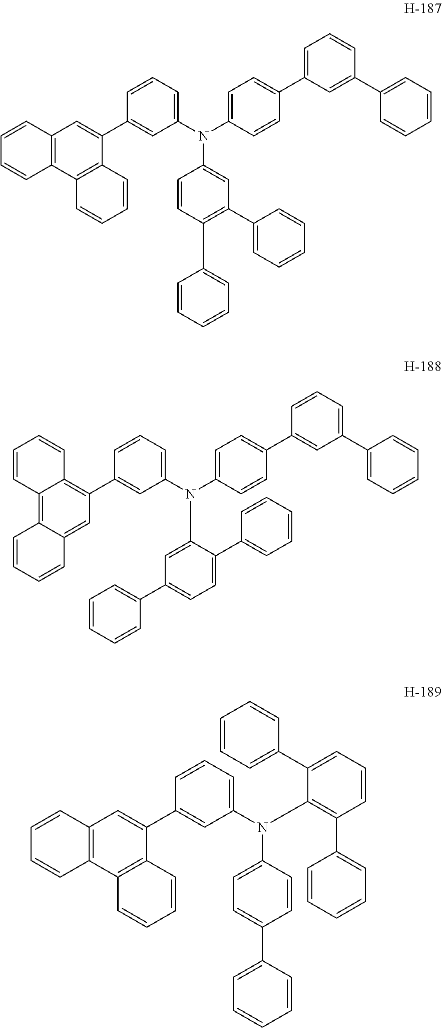 [SMILES: c1ccc(-c2cccc(-c3ccc(N(c4cccc(-c5cc6ccccc6c6ccccc56)c4)c4ccc(-c5ccccc5)c(-c5ccccc5)c4)cc3)c2)cc1 ; c1ccc(-c2cccc(-c3ccc(N(c4cccc(-c5cc6ccccc6c6ccccc56)c4)c4cc(-c5ccccc5)ccc4-c4ccccc4)cc3)c2)cc1 ; c1ccc(-c2ccc(N(c3cccc(-c4cc5ccccc5c5ccccc45)c3)c3c(-c4ccccc4)cccc3-c3ccccc3)cc2)cc1] 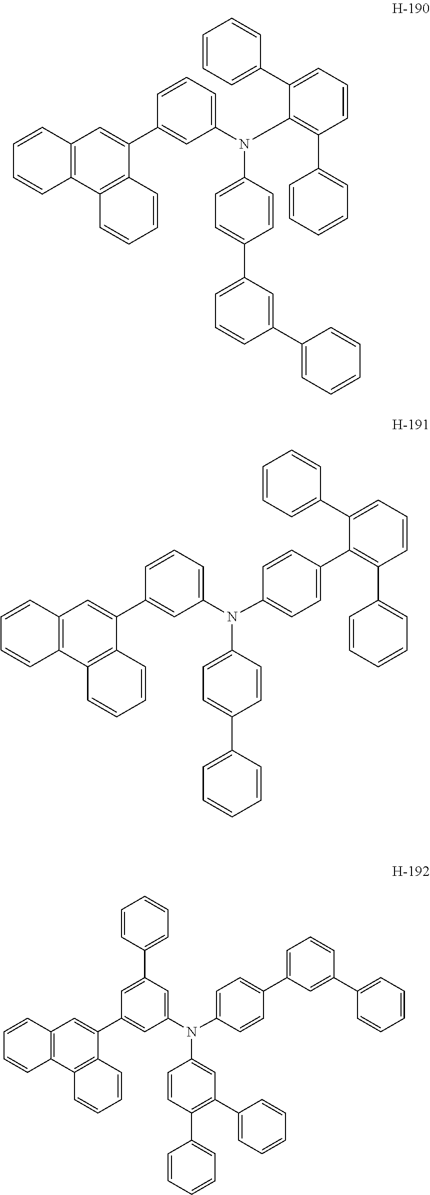 [SMILES: c1ccc(-c2cccc(-c3ccc(N(c4cccc(-c5cc6ccccc6c6ccccc56)c4)c4c(-c5ccccc5)cccc4-c4ccccc4)cc3)c2)cc1 ; c1ccc(-c2ccc(N(c3ccc(-c4c(-c5ccccc5)cccc4-c4ccccc4)cc3)c3cccc(-c4cc5ccccc5c5ccccc45)c3)cc2)cc1 ; c1ccc(-c2cccc(-c3ccc(N(c4cc(-c5ccccc5)cc(-c5cc6ccccc6c6ccccc56)c4)c4ccc(-c5ccccc5)c(-c5ccccc5)c4)cc3)c2)cc1] 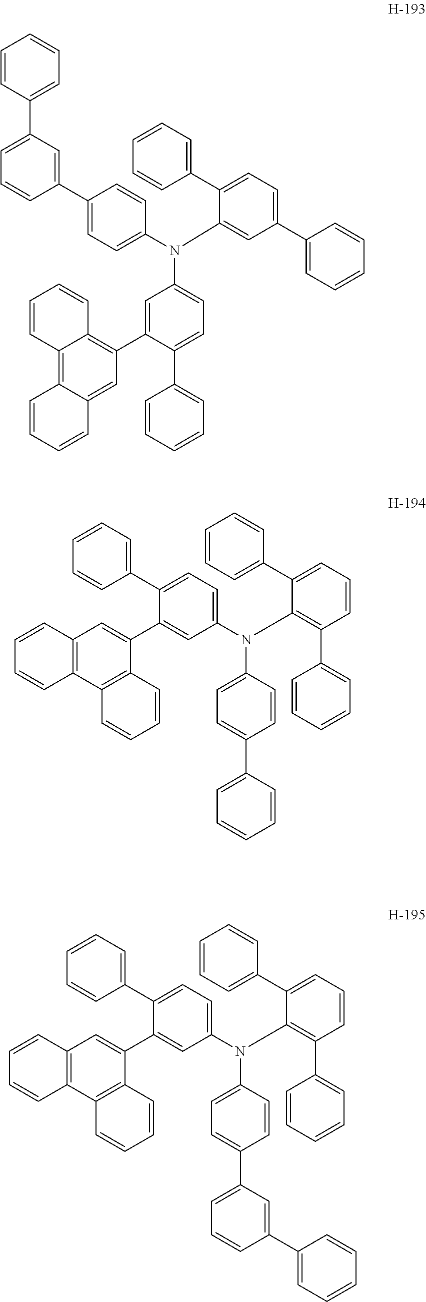 [SMILES: c1ccc(-c2cccc(-c3ccc(N(c4ccc(-c5ccccc5)c(-c5cc6ccccc6c6ccccc56)c4)c4cc(-c5ccccc5)ccc4-c4ccccc4)cc3)c2)cc1 ; c1ccc(-c2ccc(N(c3ccc(-c4ccccc4)c(-c4cc5ccccc5c5ccccc45)c3)c3c(-c4ccccc4)cccc3-c3ccccc3)cc2)cc1 ; c1ccc(-c2cccc(-c3ccc(N(c4ccc(-c5ccccc5)c(-c5cc6ccccc6c6ccccc56)c4)c4c(-c5ccccc5)cccc4-c4ccccc4)cc3)c2)cc1] 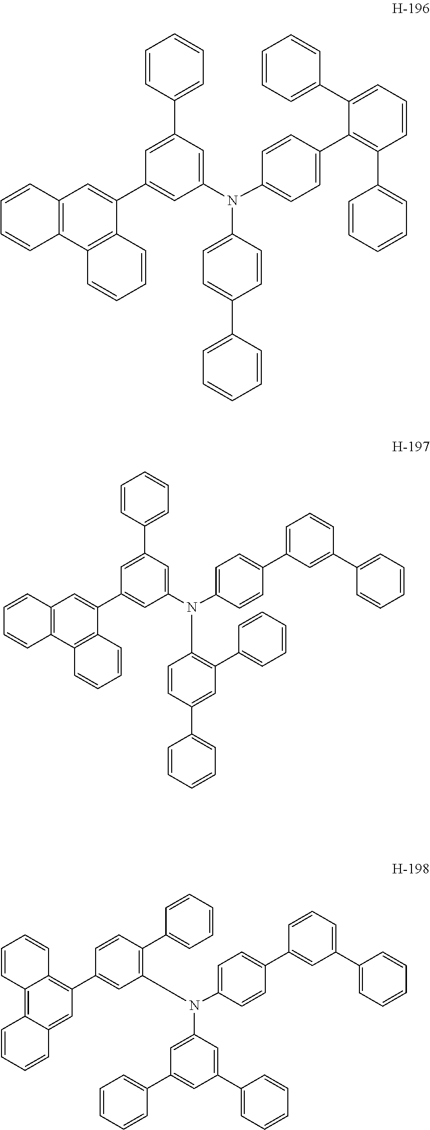 [SMILES: c1ccc(-c2ccc(N(c3ccc(-c4c(-c5ccccc5)cccc4-c4ccccc4)cc3)c3cc(-c4ccccc4)cc(-c4cc5ccccc5c5ccccc45)c3)cc2)cc1 ; c1ccc(-c2cccc(-c3ccc(N(c4cc(-c5ccccc5)cc(-c5cc6ccccc6c6ccccc56)c4)c4ccc(-c5ccccc5)cc4-c4ccccc4)cc3)c2)cc1 ; c1ccc(-c2cccc(-c3ccc(N(c4cc(-c5ccccc5)cc(-c5ccccc5)c4)c4cc(-c5cc6ccccc6c6ccccc56)ccc4-c4ccccc4)cc3)c2)cc1] 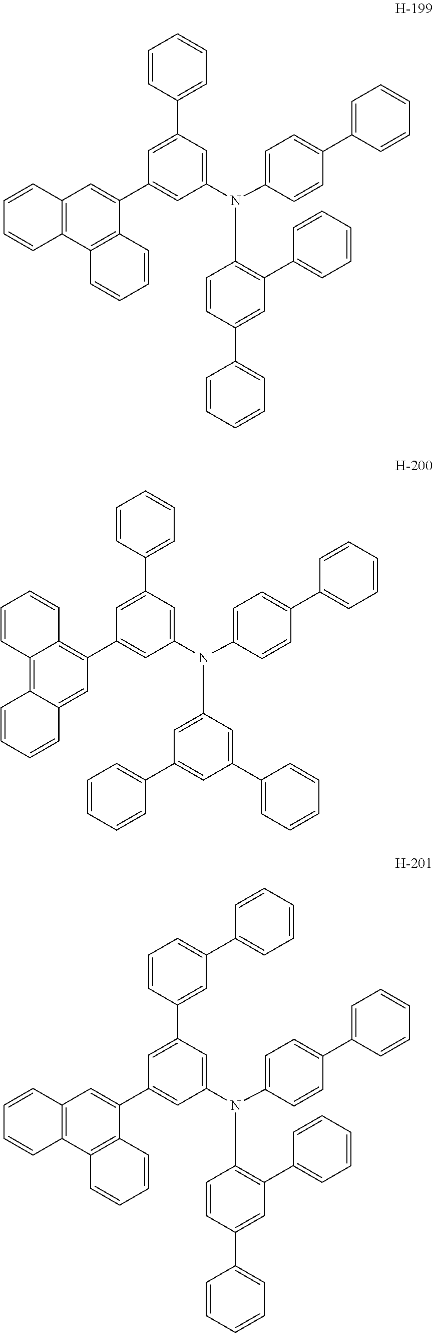 [SMILES: c1ccc(-c2ccc(N(c3cc(-c4ccccc4)cc(-c4cc5ccccc5c5ccccc45)c3)c3ccc(-c4ccccc4)cc3-c3ccccc3)cc2)cc1 ; c1ccc(-c2ccc(N(c3cc(-c4ccccc4)cc(-c4ccccc4)c3)c3cc(-c4ccccc4)cc(-c4cc5ccccc5c5ccccc45)c3)cc2)cc1 ; c1ccc(-c2ccc(N(c3cc(-c4cccc(-c5ccccc5)c4)cc(-c4cc5ccccc5c5ccccc45)c3)c3ccc(-c4ccccc4)cc3-c3ccccc3)cc2)cc1] 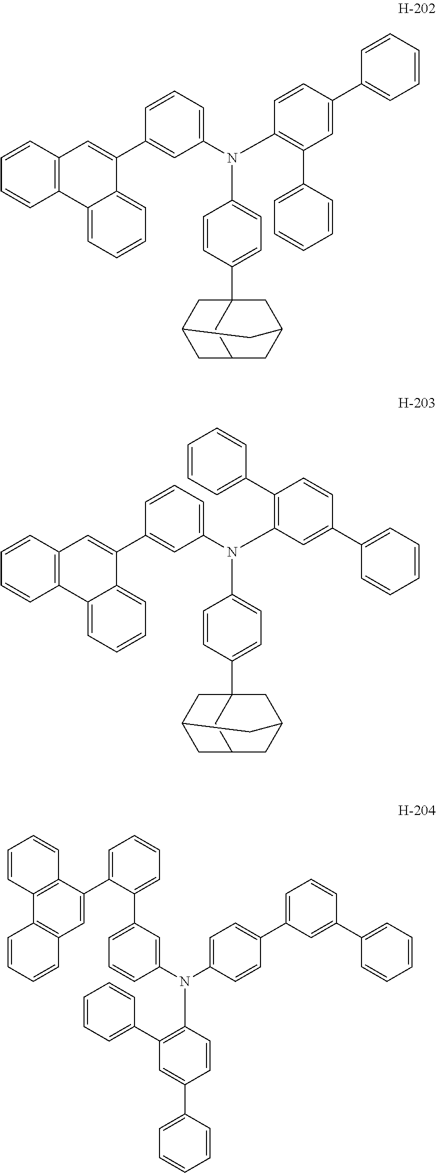 [SMILES: c1ccc(-c2ccc(N(c3ccc(C45CC6CC(CC(C6)C4)C5)cc3)c3cccc(-c4cc5ccccc5c5ccccc45)c3)c(-c3ccccc3)c2)cc1 ; c1ccc(-c2ccc(-c3ccccc3)c(N(c3ccc(C45CC6CC(CC(C6)C4)C5)cc3)c3cccc(-c4cc5ccccc5c5ccccc45)c3)c2)cc1 ; c1ccc(-c2cccc(-c3ccc(N(c4cccc(-c5ccccc5-c5cc6ccccc6c6ccccc56)c4)c4ccc(-c5ccccc5)cc4-c4ccccc4)cc3)c2)cc1] 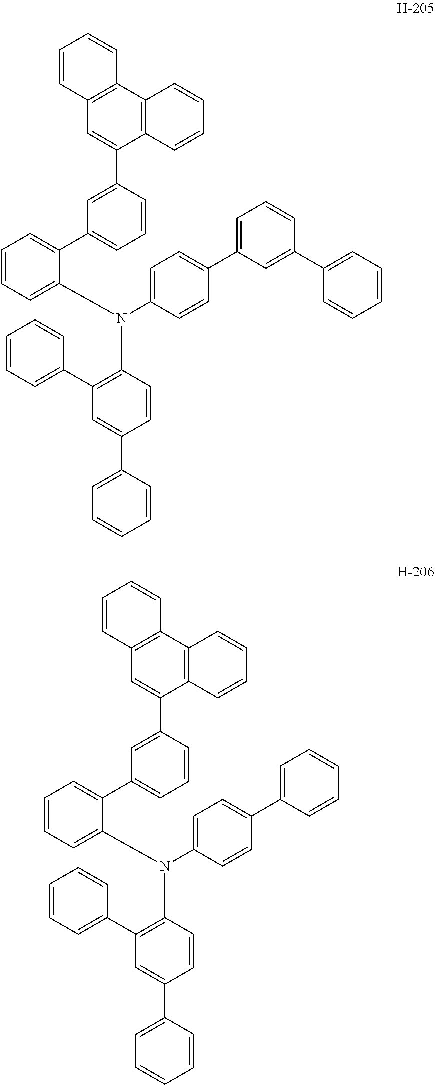 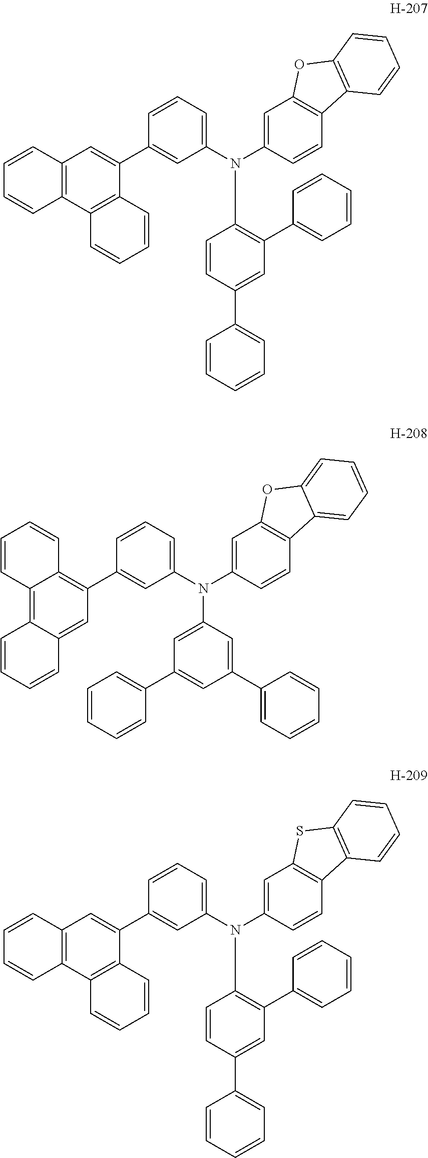 [SMILES: c1ccc(-c2ccc(N(c3cccc(-c4cc5ccccc5c5ccccc45)c3)c3ccc4c(c3)oc3ccccc34)c(-c3ccccc3)c2)cc1 ; c1ccc(-c2cc(-c3ccccc3)cc(N(c3cccc(-c4cc5ccccc5c5ccccc45)c3)c3ccc4c(c3)oc3ccccc34)c2)cc1 ; c1ccc(-c2ccc(N(c3cccc(-c4cc5ccccc5c5ccccc45)c3)c3ccc4c(c3)sc3ccccc34)c(-c3ccccc3)c2)cc1] 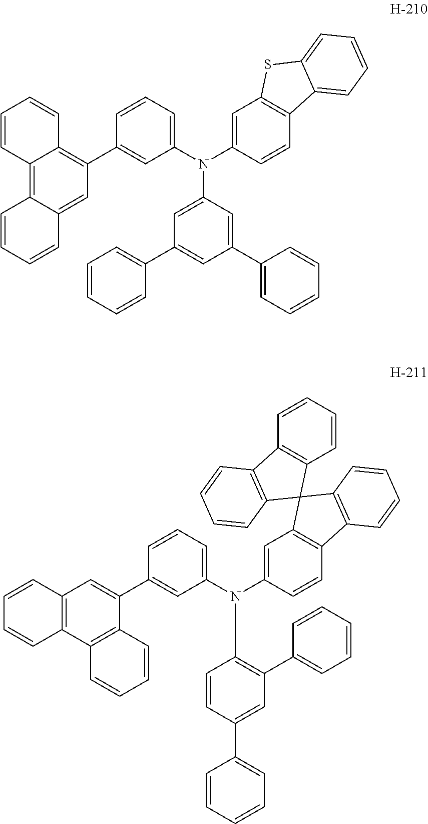 [SMILES: c1ccc(-c2cc(-c3ccccc3)cc(N(c3cccc(-c4cc5ccccc5c5ccccc45)c3)c3ccc4c(c3)sc3ccccc34)c2)cc1 ; c1ccc(-c2ccc(N(c3cccc(-c4cc5ccccc5c5ccccc45)c3)c3ccc4c(c3)C3(c5ccccc5-c5ccccc53)c3ccccc3-4)c(-c3ccccc3)c2)cc1] 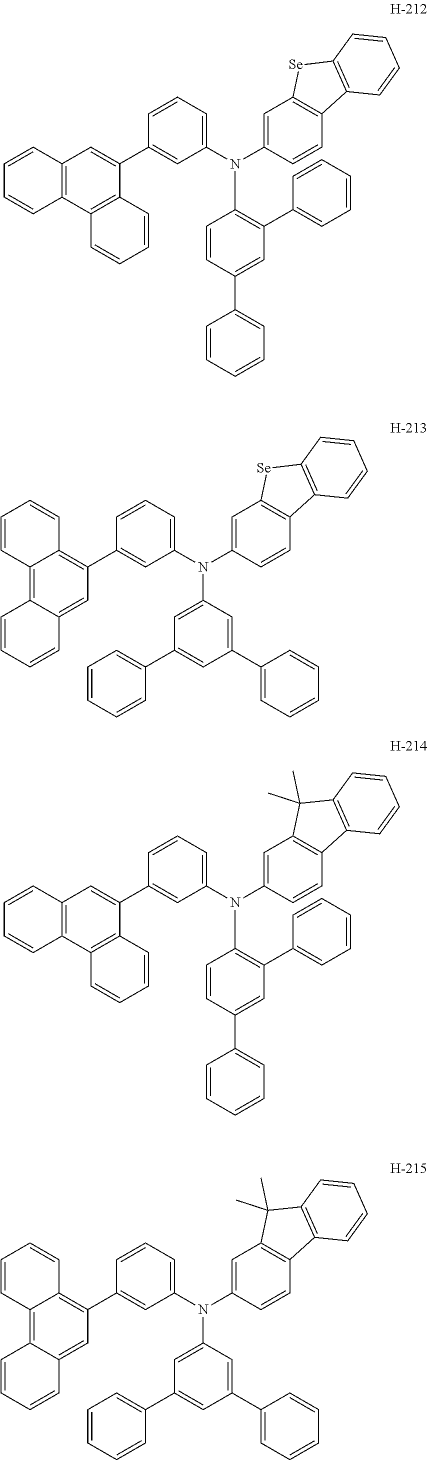 [SMILES: c1ccc(-c2ccc(N(c3cccc(-c4cc5ccccc5c5ccccc45)c3)c3ccc4c(c3)[se]c3ccccc34)c(-c3ccccc3)c2)cc1 ; c1ccc(-c2cc(-c3ccccc3)cc(N(c3cccc(-c4cc5ccccc5c5ccccc45)c3)c3ccc4c(c3)[se]c3ccccc34)c2)cc1 ; CC1(C)c2ccccc2-c2ccc(N(c3cccc(-c4cc5ccccc5c5ccccc45)c3)c3ccc(-c4ccccc4)cc3-c3ccccc3)cc21 ; CC1(C)c2ccccc2-c2ccc(N(c3cc(-c4ccccc4)cc(-c4ccccc4)c3)c3cccc(-c4cc5ccccc5c5ccccc45)c3)cc21] 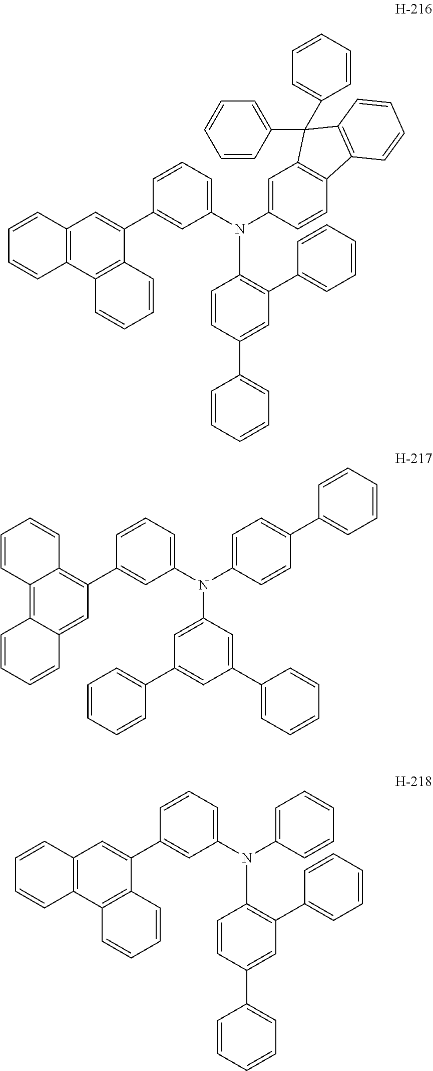 [SMILES: c1ccc(-c2ccc(N(c3cccc(-c4cc5ccccc5c5ccccc45)c3)c3ccc4c(c3)C(c3ccccc3)(c3ccccc3)c3ccccc3-4)c(-c3ccccc3)c2)cc1 ; c1ccc(-c2ccc(N(c3cc(-c4ccccc4)cc(-c4ccccc4)c3)c3cccc(-c4cc5ccccc5c5ccccc45)c3)cc2)cc1 ; c1ccc(-c2ccc(N(c3ccccc3)c3cccc(-c4cc5ccccc5c5ccccc45)c3)c(-c3ccccc3)c2)cc1] 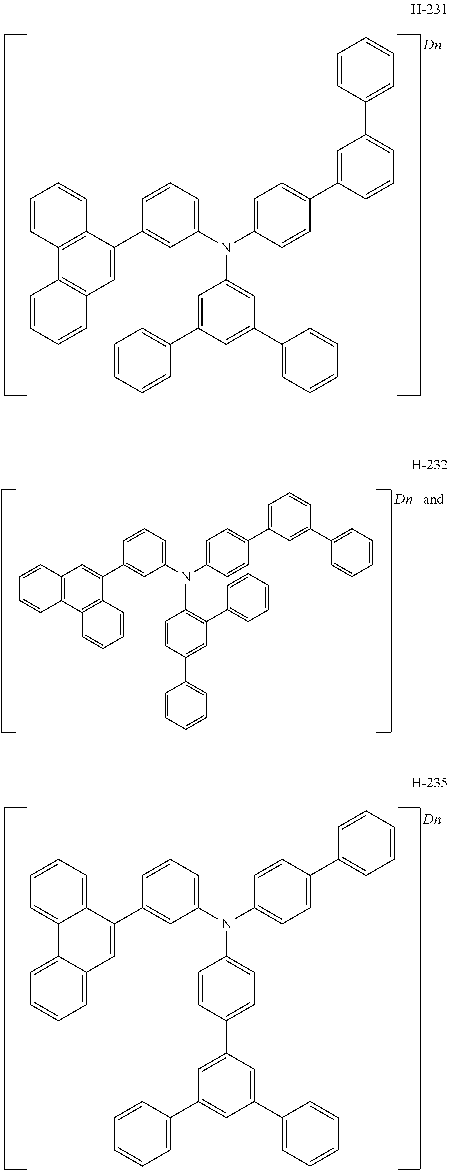
wherein in the compounds above, Dn represents that n number of hydrogens are replaced with deuterium, and n represents an integer of 1 or more, which is an integer of 1 to the maximum number of hydrogens in the compound.

20. The organic electroluminescent device comprising the organic electroluminescent compound according to claim 17.
